(19) United States
(12) Patent Application Publication
(10) Pub. No.: US 2021/0322508 A1
(43) Pub. Date: Oct. 21, 2021

(54) PEPTIDES AND COMPOSITIONS FOR TARGETED TREATMENT AND IMAGING

(71) Applicant: SIGNABLOK, INC., Shrewsbury, MA (US)

(72) Inventor: Alexander B. Sigalov, Worcester, MA (US)

(21) Appl. No.: 17/268,046

(22) PCT Filed: Aug. 13, 2019
(86) PCT No.: PCT/US19/46392

§ 371 Date: Feb. 11, 2021

Related U.S. Application Data
(60) Provisional application No. 62/717,929, filed on Aug. 13, 2018.
(60) Provisional application No. 62/751,303, filed on Oct. 26, 2018.
(60) Provisional application No. 62/836,823, filed on Apr. 22, 2019.
(60) Provisional application No. 62/843,835, filed on May 6, 2019.

(51) Int. Cl. A61K 38/08; A61K 49/00; A61P 35/00; A61K 51/04; A61K 49/08; A61K 49/14; A61K 38/12; A61K 38/17; A61K 45/06

(52) U.S. Cl. CPC A61K 38/08; A61K 49/0056; A61P 35/00; A61K 51/0497; A61K 45/06; A61K 49/14; A61K 38/12; A61K 38/177; A61K 49/085

(57) ABSTRACT

The invention disclosed herein provides compositions and methods of treating cancer and other diseases related to activated immune cells using modulators of the TREM-1/DAP-12 signaling pathway. The compositions, including peptides and peptide variants, modulate TREM-1-mediated immunological response as standalone and combination-therapy treatment regimen. Further, methods are provided for predicting the efficacy of TREM-1 modulatory therapies in patients. In one embodiment, the present invention relates to targeted treatment, prevention and/or detection of cancer including but not limited to lung cancer including non-small cell lung cancer, pancreatic cancer, giant cell tumor of the tendon sheath, tenosynovial giant cell tumor, pigmented villonodular synovitis, cancer cachexia, etc., and other cancers associated with myeloid cell activation and recruitment. Additionally, the present invention relates to the targeted treatment, prevention and/or detection of scleroderma including but not limited to calcinosis, Raynaud's phenomenon, esophageal dysmotility, scleroderma, or telangiectasia syndrome (CREST). The invention further relates to personalized medical treatments.

### CROSS-REFERENCE TO RELATED APPLICATIONS

This application claims priority to U.S. Provisional Patent Application No. 62/717,929, filed Aug. 13, 2018, U.S. Provisional Patent Application No. 62/751,303, filed Oct. 26, 2018, U.S. Provisional Patent Application No. 62/836,823, filed Apr. 22, 2019, U.S. Provisional Patent Application No. 62/843,835, filed May 6, 2019, and to U.S. Provisional Patent Application No. 62/875,287 filed Jul. 17, 2019, each of which are incorporated herein by reference in their entireties and for all purposes.

### FIELD OF THE INVENTION

The invention disclosed herein provides compositions and methods of treating cancer and other diseases related to activated immune cells using modulators of the TREM-1/DAP-12 signaling pathway. The compositions, including peptides and peptide variants, modulate TREM-1-mediated immunological response as standalone and combination-therapy treatment regimen. Further, methods are provided for predicting the efficacy of TREM-1 modulatory therapies in patients. In one embodiment, the present invention relates to targeted treatment, prevention and/or detection of cancer including but not limited to lung cancer including non-small cell lung cancer, pancreatic cancer, giant cell tumor of the tendon sheath, tenosynovial giant cell tumor, pigmented villonodular synovitis, cancer cachexia, etc., and other cancers associated with myeloid cell activation and recruitment. Additionally, the present invention relates to the targeted treatment, prevention and/or detection of scleroderma including but not limited to calcinosis, Raynaud's phenomenon, esophageal dysmotility, scleroderma, or telangiectasia syndrome (CREST). The invention further relates to personalized medical treatments.

### BACKGROUND OF THE INVENTION

Administration of therapeutic peptides often causes activation of nontarget cells and leads to undesired side effects and increases risk of undesired immunogenic effects. Limitations generally attributed to therapeutic peptides are: a short half-life in the circulation because of their rapid degradation by proteolytic enzymes of the digestive system and blood plasma; rapid removal from the circulation by the liver (hepatic clearance) and kidneys (renal clearance); poor ability to cross physiological barriers, such as the blood-brain barrier. Because of therapeutic peptides having general hydrophilicity; high conformational flexibility, and use resulting sometimes in a lack of selectivity involving interactions with different receptors/targets (poor specific biodistribution), described in part in Vlieghe, et al. Drug Discov Today 2010, 15:40-56.

Consequently, there is need for more effective formulations of therapeutic peptides to improve their targeted delivery, prolonged circulatory half-life, biocompatibility and therapeutic efficiency.

### SUMMARY OF THE INVENTION

The invention disclosed herein provides compositions and methods of treating cancer and other diseases related to activated immune cells using modulators of the TREM-1/DAP-12 signaling pathway. The compositions, including peptides and peptide variants, modulate TREM-1-mediated immunological response as standalone and combination-therapy treatment regimen. Further, methods are provided for predicting the efficacy of TREM-1 modulatory therapies in patients. In one embodiment, the present invention relates to targeted treatment, prevention and/or detection of cancer including but not limited to lung cancer including non-small cell lung cancer, pancreatic cancer, giant cell tumor of the tendon sheath, tenosynovial giant cell tumor, pigmented villonodular synovitis, cancer cachexia, etc., and other cancers associated with myeloid cell activation and recruitment. Additionally, the present invention relates to the targeted treatment, prevention and/or detection of scleroderma including but not limited to calcinosis, Raynaud's phenomenon, esophageal dysmotility, scleroderma, or telangiectasia syndrome (CREST). The invention further relates to personalized medical treatments.

The present disclosure describes novel amphipathic trifunctional peptides and therapeutic compositions comprising such trifunctional peptides for use in treating diseases related to activated immune cells. In some embodiments, each trifunctional peptide is capable of at least three functions: 1) mediating formation of naturally long half-life lipopeptide/lipoprotein particles upon interaction with lipoproteins, 2) facilitation of the targeted delivery to cells of interest and/or sites of disease, and 3) treatment, prevention, and/or detection of a disease or condition. In some embodiments, each trifunctional peptide is capable of at least three functions: 1) mediating the self-assembly of naturally long half-life lipopeptide particles upon binding to lipid or lipid mixtures, 2) facilitation of the targeted delivery to cells of interest and/or sites of disease, and 3) treatment, prevention, and/or detection of a disease or condition. In certain embodiments, the present invention relates to amphipathic trifunctional peptides consisting of two amino acid domains, wherein upon interaction with plasma lipoproteins, one amino acid domain mediates formation of naturally long half-life lipopeptide/lipoprotein particles and targets these particles to macrophages, whereas the other amino acid domain inhibits the TREM-1/DAP-12 receptor signaling complex expressed on macrophages. The invention further relates to personalized medical treatments for cancer that involve targeting specific cancers by their tumor environment. The invention further relates to personalized medical treatments for scleroderma (systemic sclerosis, SSc). More specifically, the invention provides for treatment of scleroderma or a related autoimmune or a fibrotic condition by using modulators of the TREM-1/DAP-12 pathway standalone or together with other antifibrotic therapies and the use of such combinations in the treatment of scleroderma.

In some embodiments, the invention provides a method for treating cancer in a patient in need thereof, said method comprising administering to said patient a therapeutically effective amount of at least one modulator that is effective for modulating the TREM-1/DAP-12 signaling pathway together with a therapeutically amount of an anticancer vaccine, an anticancer immunotherapy agent, anti-cancer immunomodulatory agent, an additional anticancer therapeutic, radiation therapy, surgery or a combination thereof. In one embodiment, said method further comprises administering said modulator together with a pharmaceutically acceptable excipient, carrier, diluents, or a combination thereof. In some embodiments, said carrier is selected from the group consisting of lipids, proteins or polypeptides, and mixtures thereof. In one embodiment, said method further comprises prior to administering the first dose of said modulator, the subject received a prior therapy selected from the group consisting of an anticancer vaccine, an anticancer immunotherapy agent, anti-cancer immunomodulatory agent, an additional anticancer therapeutic, radiation therapy, surgery or a combination thereof. However it is not meant to limit such prior therapies. In some embodiments, said cancer recurred or progressed after the prior therapy. In some embodiments, said administration of said modulator to said patient is continued as a long-term maintenance treatment for duration between about two weeks to about five years, preferably said administration is continued for duration of up to one year. In some embodiments, said anticancer vaccine is selected from the group consisting of Gardasil, Cervarix, Sipuleucel-T/Provenge, and the like. In some embodiments, said anticancer immunotherapy agent is selected from the group consisting of Alemtuzumab, Ipilimumab, Ofatumumab, Nivolumab, Pembrolizumab, Rituximab, Blinatumomab, Daratumumab, Trastuzumab, Cetuximab, Elotuzumab, adoptive T-cell therapy, T-Vec, Interferon, Interleukin, and a combination thereof. In some embodiments, said anticancer immunomodulatory agent is selected from the group consisting of thalidomide, lenolidomide, pomalidomide, and a combination thereof. In some embodiments, said additional anticancer therapeutic is selected from the group consisting of an alkylating agent, a tubulin inhibitor, a topoisomerase inhibitor, proteasome inhibitor, a CHK1 inhibitor, a CHK2 inhibitor, a PARP inhibitor, a tyrosine kinase inhibitor, CSF-1/CSF-1R inhibitor, doxorubicin, gemcitabine, entrectinib, epirubicin, vinblastine, etoposide, topotecan, bleomycin, mytomycin c, and the like. In some embodiments, said alkylating agent is selected from the group consisting of Dacarbazine, Procarbazine, Carmustine, Lomustine, Uramustine, BuSulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, Oxaliplatin, and the like. In some embodiments, said tubulin inhibitor is selected from the group consisting of Taxol, Docetaxel, Abraxane, Vinblastin, Epothilone, Colchicine, Cryptophycin, BMS 347550, Rhizoxin, Ecteinascidin, Dolastin 10, Cryptophycin 52, IDN-5109, and the like. In some embodiments, said topoisomerase inhibitor is a topoisomerase I inhibitor selected from the group consisting of Irinotecan, Topotecan, Camptothecins (CPT), and the like. In some embodiments, said topoisomerase inhibitor is a topoisomerase II inhibitor selected from the group consisting of Amsacrine, Etoposide, Teniposide, Epipodophyllotoxins, ellipticine, and the like. In some embodiments, said proteasome inhibitor is selected from the group consisting of Velcade (bortezomib), and Kyprolis (carfilzomib), and the like. In some embodiments, said CHK1 inhibitor is selected from the group consisting of TCS2312, PF-0047736, AZ07762, A-69002, and A-641397, and the like. In some embodiments, said PARP inhibitor is selected from the group consisting of Olaparib, Talazoparib, ABT-888, (veliparib), KU-59436, AZD-2281, AG-014699, BSI-201, BGP-15, INO-1001, ONO-2231, and the like. In some embodiments, said tyrosine kinase inhibitor is selected from the group consisting of pexidartinib, entrectinib, matinib mesylate (STI571; Gleevec), gefitinib (Iressa), erlotinib (OSI-1774; Tarceva), lapatinib (GW-572016), canertinib (CI-1033), semaxinib (SU5416), vatalanib (PTK787/ZK222584), sorafenib (BAY 43-9006), sutent (SU11248), and leflunomide (SU101), and the like. In some embodiments, said CSF-1/CSF-1R inhibitor is selected from the group consisting of CSF-1R kinase inhibitor, an antibody that binds CSF-1R and is capable of blocking binding of CSF-1 and/or IL-34 to CSF-1R, and the like. In some embodiments, said CSF-1R kinase inhibitor is imatinib, nilotinib or PLX3397. In some embodiments, said radiation therapy is selected from the group consisting of X-rays, ion beams, electron beams, gamma-rays, UV-rays, and decay of a radioactive isotope, or a combination thereof. In some embodiments, said surgery is surgical tumor resection. In some embodiments, said cancer is lung cancer including non-small cell lung cancer, pancreatic cancer, breast cancer, liver cancer, multiple myeloma, melanoma, leukemia, central nervous system cancer, stomach cancer, prostate, colon cancer, colorectal cancer, brain cancer, gastrointestinal cancer, gastric cancer, ovarian cancer, renal cancer, skin cancer, osteosarcoma, endometrial cancer, esophageal cancer, kidney cancer, prostate cancer, thyroid cancer, neuroblastoma, neurofibroma, glioma, glioblastoma, glioblastoma multiforme, stomach cancer, bladder cancer, head and neck cancer, cervical cancer, giant cell tumor of the tendon sheath, tenosynovial giant cell tumor, pigmented villonodular synovitis and other cancers in which myeloid cells are involved or recruited and cancer cachexia. In some embodiments, said at least one said modulator comprises a variant peptide sequence that is capable of binding TREM-1/DAP-12 and reducing or blocking TREM-1/DAP-12 activity (signaling and/or activation). In some embodiments, said variant peptide sequence comprises at least one D-amino acid. In some embodiments, said variant peptide sequence is a cyclic peptide. In some embodiments, said variant peptide sequence is derived from transmembrane domain sequences of human or animal TREM-1 and/or its signaling subunit, DAP-12, or a combination thereof. In some embodiments, said variant peptide sequence comprises LR12 and/or LP17 peptide variants and the like or a combination thereof. In some embodiments, said modulator comprises at least one isolated antibody or fragment thereof, that is capable of specifically binding TREM-1/DAP-12 and which is capable of reducing or blocking TREM-1/DAP-12 activity (signaling and/or activation). In one embodiment, said method further comprises a diagnostic method. In one embodiment, said diagnostic method is performed prior to administering the first dose of said modulator to predict response of said patient to a therapy of the method of claim 1. In some embodiments, said diagnostic method comprises isolating a biological sample from said patient and determining in said sample the expression of CSF-1, CSF-1R, IL-6, TREM-1 and/or number of CD68-positive cells or a combination thereof, wherein the higher is the expression level of CSF-1, CSF-1R, IL-6, TREM-1 or the higher is number of CD68-positive cells or a combination thereof, the better the patient is predicted to respond to a therapy of the method of claim 1. In some embodiments, said method comprises: (a) administering to said patient an amount of at least one said modulator of the method of claim 1 that is capable of binding TREM-1 and is conjugated to at least one imaging probe, or a combination thereof, in a detectably effective amount; (b) imaging at least a portion of the patient; (c) detecting the labeled probe, wherein the location and amount of the labeled probe corresponds to at least one symptom of the myeloid cell-related cancer condition and correlates with the TREM-1 expression levels and the higher the levels are, the better the patient is predicted to respond to a therapy of the method of claim 1. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol, or a combination thereof.

The present invention encompasses the discovery that it is possible to combine multiple functions in one amphipathic polypeptide amino acid sequence to confer a variety of properties on the resulting peptide and provides novel peptides and compounds, which are capable of executing at least, three functions: 1) mediation of formation of naturally long half-life lipopeptide/lipoprotein particles (LP) upon interaction with native lipoproteins, 2) facilitation of the targeted delivery to cells of interest and/or sites of disease, and 3) treatment, prevention, and/or detection of a disease or condition. In one embodiment, said peptides and compounds of the present invention are used in combinations thereof. The peptides and compounds of the present invention and combinations thereof have a wide variety of uses, particularly in the areas of oncology, transplantology, dermatology, hepatology, ophthalmology, cardiovascular diseases, sepsis, autoimmune diseases, neurodegenerative diseases and other diseases and conditions. They also are useful in the production of medical devices (for example, medical implants and implantable devices).

In some embodiments, the invention provides a synthetic trifunctional peptide comprising: (a) a first amino acid domain that does not interact with native lipoproteins in isolated form, wherein said first amino acid domain is at least 3 amino acids in length and is capable of treating, preventing and/or detecting an immune-related disease or condition; and (b) a second amino acid domain that mediates formation of lipopeptide/lipoprotein particles upon interaction of the peptide with native lipoproteins and targets these particles to cells of interest and/or sites of disease or condition, which second amino acid domain is at least 6 amino acids in length and has an amphipathic alpha helical amino acid sequence. In some embodiments, said first amino acid domain comprises amino acid sequence Gly-Phe-Leu-Ser-Lys-Ser-Leu-Val-Phe, wherein Gly is glycine, Phe is phenylalanine, Leu is leucine, Ser is serine, Lys is lysine, and Val is valine. In some embodiments, said first amino acid domain comprises amino acid sequence Met-Trp-Lys-Thr-Pro-Thr-Leu-Lys-Tyr-Phe, wherein Met is methionine, Trp is tryptophan, Lys is lysine, Thr is threonine, Pro is proline, Leu is leucine, Tyr is tyrosine, and Phe is phenylalanine. In some embodiments, said second amino acid domain comprises amino acid sequence Trp-Gln-Glu-Glu-Met-Glu-Leu-Tyr-Arg-Gln-Lys-Val, wherein Tyr is tyrosine, Leu is leucine, Gln is glutamine, Lys is lysine, Trp is tryptophan, Glu is glutamic acid, Met is methionine, Arg is arginine, and Val is valine. In some embodiments, said second amino acid domain comprises amino acid sequence Gly-Glu-Glu-Met-Arg-Asp-Arg-Ala-Arg-Ala-His-Val, wherein Gly is glycine, Glu is glutamic acid, Met is methionine, Arg is arginine, Asp, asparagine, Ala is alanine, His is histidine, and Val is valine. In some embodiments, said first amino acid domain and/or said second amino acid domain are conjugated to at least one imaging probe.

In some embodiments, the invention provides a method of imaging an immune-related disease or condition, comprising a) providing; i) a patient having at least one symptom of a disease or condition in which immune cells are involved or recruited, and ii) a compound of claim 8, wherein the composition has an affinity for immune receptors; b) administering said composition to said patient in a detectably effective amount, c) imaging at least a portion of the patient; and d) detecting the labeled probe, wherein the location of the labeled probe corresponds to at least one symptom of the immune-related disease or condition.

In some embodiments, the invention provides a method of treating an immune-related disease or condition, comprising: a) providing; i) a patient having at least one symptom of a disease or condition in which immune cells are involved or recruited, and ii) the composition of claim 1 capable of modulating immune receptors; b) administering said composition to said patient under conditions such that said at least one symptom is reduced. In some embodiments, said immune-related disease or condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, empyema, acute mesenteric ischemia, hemorrhagic shock, multiple sclerosis, autoimmune diseases (e.g., rheumatoid arthritis, psoriatic arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, sepsis, inflammatory lung diseases (e.g., interstitial pneumonitis and asthma), retinopathy (e.g., retinopathy of prematurity and diabetic retinopathy), neurodegenrative diseases (e.g., Alzheimer's, Parkinson's and Huntington's diseases), gastroenterological diseases and conditions (e.g. inflammatory bowel disease, Crohn's disease, celiac disease), Guillain-Barre syndrome, Hashimoto's disease, pernicious anemia, primary biliary cirrhosis, chronic active hepatitis, alcohol-induced liver disease, nonalcoholic fatty liver disease and non-alcoholic steatohepatitis, skin problems (e.g. atopic dermatitis, psoriasis, pemphigus vulgaris), cardiovascular problems (e.g. autoimmune pericarditis), allergic diathesis (e.g. delayed type hypersensitivity), contact dermatitis, herpes simplex/zoster, respiratory conditions (e.g. allergic alveolitis), inflammatory conditions (e.g. myositis), ankylosing spondylitis, tissue/organ transplant (e.g., heart/lung transplants) rejection reactions, brain and spinal cord injuries, and other diseases and conditions where immune cells are involved or recruited.

In some embodiments, the invention provides a synthetic trifunctional peptide comprising: (a) a first amino acid domain that does not interact with native lipoproteins in isolated form, which first amino acid domain is at least 3 amino acids in length and is capable of treating, preventing and/or detecting an immune-related disease or condition; and (b) a second amino acid domain that mediates formation of lipopeptide/lipoprotein particles upon interaction of the peptide with native lipoproteins and targets these particles to cells of interest and/or sites of disease or condition, which second amino acid domain is at least 6 amino acids in length and has an amphipathic alpha helical amino acid sequence. In some embodiments, said first amino acid domain comprises amino acid sequence Gly-Phe-Leu-Ser-Lys-Ser-Leu-Val-Phe, wherein Gly is glycine, Phe is phenylalanine, Leu is leucine, Ser is serine, Lys is lysine, and Val is valine. In some embodiments, said first amino acid domain comprises amino acid sequence Met-Trp-Lys-Thr-Pro-Thr-Leu-Lys-Tyr-Phe, wherein Met is methionine, Trp is tryptophan, Lys is lysine, Thr is threonine, Pro is proline, Leu is leucine, Tyr is tyrosine, and Phe is phenylalanine. In some embodiments, said second amino acid domain comprises amino acid sequence Trp-Gln-Glu-Glu-Met-Glu-Leu-Tyr-Arg-Gln-Lys-Val, wherein Tyr is tyrosine, Leu is leucine, Gln is glutamine, Lys is lysine, Trp is tryptophan, Glu is glutamic acid, Met is methionine, Arg is arginine, and Val is valine. In some embodiments, said the second amino acid domain comprises amino acid sequence Gly-Glu-Glu-Met-Arg-Asp-Arg-Ala-Arg-Ala-His-Val, wherein Gly is glycine, Glu is glutamic acid, Met is methionine, Arg is arginine, Asp, asparagine, Ala is alanine, His is histidine, and Val is valine. In some embodiments, said the first amino acid domain and/or the second amino acid domain are conjugated to at least one imaging probe.

In some embodiments, the invention provides a method of imaging an immune-related disease or condition, comprising a) providing; i) a patient having at least one symptom of a disease or condition in which immune cells are involved or recruited, and ii) a compound of claim 8, wherein the composition has an affinity for immune receptors; b) administering said composition to said patient in a detectably effective amount c) imaging at least a portion of the patient; and d) detecting the labeled probe, wherein the location of the labeled probe corresponds to at least one symptom of the immune-related disease or condition.

In some embodiments, the invention provides a method of treating an immune-related disease or condition, comprising: a) providing; i) a patient having at least one symptom of a disease or condition in which immune cells are involved or recruited, and ii) the composition of claim 1 capable of modulating immune receptors; b) administering said composition to said patient under conditions such that said at least one symptom is reduced. In some embodiments, said immune-related disease or condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, empyema, acute mesenteric ischemia, hemorrhagic shock, multiple sclerosis, autoimmune diseases (e.g., rheumatoid arthritis, psoriatic arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, sepsis, inflammatory lung diseases (e.g., interstitial pneumonitis and asthma), retinopathy (e.g., retinopathy of prematurity and diabetic retinopathy), neurodegenrative diseases (e.g., Alzheimer's, Parkinson's and Huntington's diseases), gastroenterological diseases and conditions (e.g. inflammatory bowel disease, Crohn's disease, celiac disease), Guillain-Barre syndrome, Hashimoto's disease, pernicious anemia, primary biliary cirrhosis, chronic active hepatitis, alcohol-induced liver disease, nonalcoholic fatty liver disease and non-alcoholic steatohepatitis, skin problems (e.g. atopic dermatitis, psoriasis, pemphigus vulgaris), cardiovascular problems (e.g. autoimmune pericarditis), allergic diathesis (e.g. delayed type hypersensitivity), contact dermatitis, herpes simplex/zoster, respiratory conditions (e.g. allergic alveolitis), inflammatory conditions (e.g. myositis), ankylosing spondylitis, tissue/organ transplant (e.g., heart/lung transplants) rejection reactions, and other diseases and conditions where immune cells are involved or recruited.

The present disclosure provides novel peptides and compounds, which are capable of executing three functions: 1) assistance in the self-assembly of naturally long half-life lipopeptide particles upon interaction with lipoproteins, 2) facilitation of the targeted delivery to cells of interest and/or sites of disease, and 3) treatment, prevention, and/or detection of a disease or condition. In one embodiment, said peptides and compounds of the present invention form synthetic lipopeptide particles upon binding to lipid or lipid mixtures.

In some embodiments, the invention provides a synthetic trifunctional polypeptide comprising at least one peptide domain of 3 to 35 amino acids in length having a C-terminal amino acid and at least one amphipathic domain of 6 to 45 to amino acids in length comprising an amphipathic lipopeptide having an N-terminal amino acid, wherein said first domain's C-terminal amino acid is attached to said second domain's N-terminal amino acid. In one embodiment, said synthetic trifunctional polypeptide further comprises an imaging agent. In one embodiment, said synthetic trifunctional polypeptide further comprises a therapeutic agent. In one embodiment, said synthetic trifunctional polypeptide further comprises a targeting agent. In one embodiment, said synthetic trifunctional polypeptide further comprises a lipopeptide nanoparticle.

In some embodiments, the invention provides a population of spherical lipopeptide nanoparticles or discoidal lipopeptide nanoparticles comprising a plurality of synthetic trifunctional polypeptides, wherein said synthetic trifunctional polypeptide comprising at least one peptide domain of 3 to 35 amino acids in length having a C-terminal amino acid and at least one amphipathic domain of 6 to 45 to amino acids in length comprising an amphipathic lipopeptide having an N-terminal amino acid, wherein said first domain's C-terminal amino acid is attached to said second domain's N-terminal amino acid.

In some embodiments, the invention provides a method of treating an immune-related disease or condition, comprising: a) providing; i) a patient having at least one symptom of a disease or condition in which immune cells are involved or recruited, and ii) a synthetic trifunctional polypeptide comprising at least one peptide domain of 3 to 35 amino acids in length having a C-terminal amino acid and at least one amphipathic domain of 6 to 45 to amino acids in length comprising an amphipathic lipopeptide having an N-terminal amino acid, wherein said first domain's C-terminal amino acid is attached to said second domain's N-terminal amino acid, wherein said trifunctional polypeptide is capable of modulating immune receptors; b) administering said synthetic trifunctional polypeptide to said patient under conditions such that said at least one symptom is reduced.

The invention relates to personalized medical treatments for cancer that involve targeting specific cancers by their tumor environment. More specifically, the invention provides for treatment of various cancers by using inhibitors of the TREM-1/DAP-12 pathway. These inhibitors include peptide variants and compositions that modulate the TREM-1-mediated immunological responses beneficial for the treatment of cancer. In addition, the invention provides for predicting the efficacy of TREM-1-targeted therapies in various cancers by analyzing biological samples for the presence of myeloid cells and for the TREM-1 expression levels. In one embodiment, the peptides and compositions of the present invention modulate TREM-1/DAP-12 receptor complex expressed on macrophages. In one embodiment, the peptides and compositions of the invention are conjugated to an imaging probe. In one embodiment, the invention provides for detecting the TREM-1-expressing cells and tissues in an individual with cancer using imaging techniques and the peptides and compositions of the invention containing an imaging probe. In one embodiment, the peptides and compositions of the invention are used in combinations thereof. In one embodiment, the peptides and compositions of the invention are used in combinations with other anticancer therapeutic agents. In one embodiment, the present invention relates to the targeted treatment, prevention and/or detection of cancer including but not limited to pancreatic cancer, breast cancer, liver cancer, multiple myeloma, leukemia, bladder cancer, CNS cancer, stomach cancer, prostate, colorectal cancer, brain cancer, ovarian cancer, renal cancer, skin cancer, osteosarcoma and other cancers and cancer cachexia.

The invention provides for a method of treating cancer in an individual in need thereof by administering to the individual an effective amount of an inhibitor of the TREM-1/DAP-12 pathway. In one aspect, the inhibitors are selected from peptide variants and compositions that suppress tumor growth by modulating the TREM-1/DAP-12 signaling pathway. In one embodiment, any or both the domains comprise minimal biologically active amino acid sequence. In one embodiment, the peptide variant comprises a cyclic peptide sequence. In one embodiment, the peptide variant comprises a disulfide-linked dimer. In one embodiment, the peptide variant includes amino acids selected from the group of natural and unnatural amino acids including, but not limited to, L-amino acids, or D-amino acids. In one embodiment, an imaging probe and/or an additional therapeutic agent is conjugated to the peptide variants and compositions of the invention. In one embodiment, the imaging agent is a Gd-based contrast agent (GBCA) for magnetic resonance imaging (MRI). In one embodiment, the imaging agent is a [⁶⁴Cu]-containing imaging probe for imaging systems such as a positron emission tomography (PET) imaging systems (and combined PET/computer tomography (CT) and PET/MRI systems).

In one embodiment, the peptides and compositions of the invention are used in combinations thereof. In one embodiment, the peptides and compositions of the invention are used in combinations with other anticancer therapeutic agents. In certain embodiments, the peptide variants and compositions of the present invention are incorporated into long half-life synthetic lipopeptide particles (SLP). In certain embodiments, the peptide variants and compositions of the invention may incorporate into lipopeptide particles (LP) in vivo upon administration to the individual. In certain embodiments, the peptides and compositions of the invention can cross the blood-brain barrier (BBB), blood-retinal barrier (BRB) and blood-tumor barrier (BTB). Thus, in one aspect, the invention provides for a method for suppressing tumor growth in an individual in need thereof by administering to the individual an amount of a TREM-1 inhibitor that is effective for suppressing tumor growth.

In some embodiments, methods of treating a proliferative disorder involving a synovial joint and/or tendon sheath in a subject are provided, comprising administering to the subject an effective amount of a compound or composition that modulates TREM-1/DAP-12 activity. In some embodiments, the proliferative disorder is selected from pigmented villonodular synovitis (PVNS), giant cell tumor of the tendon sheath (GCTTS), and tenosynovial giant cell tumor (TGCT) such as diffuse type tenosynovial gian cell tumor (dtTGCT). In some embodiments, the disorder is pigmented villonodular synovitis/diffuse type tenosynovial gian cell tumor (PVNS/dtTGCT).

In some embodiments, the PVNS tumor volume is reduced by at least 30% or at least 40% or at least 50% or at least 60% or at least 70% after administration of at least two, at least three, at least four, at least five, at least six, at least seven, at least eight, at least nine, or at least ten doses of the compound or composition that modulates TREM-1/DAP-12 activity. In some embodiments, the tumor volume is tumor volume in a single joint. In some embodiments, the single join is selected from a hip joint and a knee joint. In some embodiments, the tumor volume is total tumor volume in all joints affected by PVNS. In some embodiments, the subject experiences one or more than one of the following improvements in symptoms: (a) a reduction in joint pain, (b) an increase range of motion in a joint, and (c) an increase in functional capacity of a joint, following at least one dose of the compound or composition.

In some embodiments, the compounds or compositions of the present invention are selected peptide variants and compositions (see, e.g., U.S. Pat. Nos. 9,981,004; 8,513,185; 9,815,883; 9,273,111; 8,013,116) that modulate the TREM-1/DAP-12 signaling pathway. In certain embodiments, the present invention relates to amphipathic trifunctional peptides consisting of two amino acid domains, wherein upon interaction with plasma lipoproteins, one amino acid domain mediates formation of naturally long half-life lipopeptide/lipoprotein complexes and targets these complexes to macrophages, whereas the other amino acid domain inhibits the TREM-1/DAP-12 receptor signaling complex expressed on macrophages. In one embodiment, the peptide variant comprises a cyclic peptide sequence. In one embodiment, the peptide variant comprises a disulfide-linked dimer. In one embodiment, the peptide variant includes amino acids selected from the group of natural and unnatural amino acids including, but not limited to, L-amino acids, or D-amino acids. In one embodiment, an imaging probe and/or an additional therapeutic agent is conjugated to the compounds and compositions of the invention.

In certain embodiments, the compounds and compositions of the present invention are incorporated into long half-life synthetic lipopeptide complexes (LPC). In certain embodiments, the compounds and compositions of the invention may incorporate into natural lipoprotein particles (LP) in vivo upon administration to the individual. See, e.g., US 20110256224 and (Sigalov 2014, Shen and Sigalov 2017, Shen et al. 2017, Rojas et al. 2018, Tornai et al. 2019).

In certain embodiments, the preferred TREM-1 modulatory compounds and compositions are TREM-1 inhibitory peptide sequences such e.g., as GF9 described in (described in (Sigalov 2014, Rojas et al. 2017, Shen and Sigalov 2017, Shen and Sigalov 2017) and disclosed in (U.S. Pat. Nos. 8,513,185 and 9,981,004) or LR12 and LP17 (described in Gibot, et al. Infect Immun 2006, 74:2823-2830; Gibot, et al. Shock 2009, 32:633-637; Gibot, et al. Eur J Immunol 2007, 37:456-466; Joffre, et al. J Am Coll Cardiol 2016, 68:2776-2793; Cuvier, et al. Br J Clin Pharmacol 2018, in press; Zhou, et al. Int Immunopharmacol 2013, 17:155-161; and disclosed in Faure, et al., U.S. Pat. No. 8,013,116; Faure, et al., U.S. Pat. No. 9,273,111; Gibot, et al., U.S. Pat. No. 9,657,081; Gibot and Derive, U.S. Pat. No. 9,815,883; and in Gibot and Derive, U.S. Pat. No. 9,255,136). In certain embodiments, the preferred TREM-1 modulatory compounds and compositions are antibodies that bind and block TREM-1 such e.g., as those disclosed in U.S. Pat. No. 10,189,902. In some embodiments, combinations of different TREM-1 modulatory compounds and compositions of the invention is used.

In another aspect, the invention provides for a method of predicting the efficacy of TREM-1 targeted therapies in an individual with the proliferative disorder by: (a) obtaining a biological sample from the individual; (b) determining the number of myeloid cells in the biological sample; (c) determining the expression levels of TREM-1 in the cells contained within the biological sample; (d) measuring the level of soluble form of the human TREM-1 receptor in the biological sample. See, e.g., U.S. Pat. No. 8,021,836.

In some embodiments, prior to administering the first dose of the compound or composition that modulates the TREM-1/DAP-12 receptor complex signaling, the subject receives a first therapy selected from surgical synovectomy, radiation beam therapy, radio isotope synovectomy, and joint replacement. In some embodiments, the PVNS recurred or progressed after the first therapy. In some embodiments, the compound or composition of the present invention is administered prior to a therapy selected from surgical synovectomy, radiation beam therapy, radio isotope synovectomy, and joint replacement. In some embodiments, the tumor is unresectable. In some embodiments, the subject has not received prior therapy with imatinib, nilotinib or a CSF1/CSF1R inhibitor, while in other embodiments the subject has received prior treatment with imatinib, nilotinib or a CSF1/CSF1R inhibitor. In some embodiments, the subject has not received prior treatment with a CSF1/CSF1R inhibitor, while in other embodiments the subject has received prior treatment with a CSF1/CSF1R inhibitor. In some embodiments, the compound or composition that modulates the TREM-1/DAP-12 receptor complex signaling is administered with imatinib, nilotinib, a CSF1/CSF1R inhibitor, anti-programmed cell death protein 1 (anti-PD1) or anti-programmed cell death ligand 1 (PDL1) antibodies.

In one embodiment the compound or composition of the present invention is provided as a pharmaceutical composition for intravenous administration. In one embodiment, the compound or composition of the present invention is provided as a pharmaceutical composition for oral administration. In one embodiment, the compound is administered once a day. In one embodiment, the compound is administered twice a day. In one embodiment, the method includes administering to the patient one or more additional therapeutic compounds. In one embodiment, the one or more additional therapeutic compound is selected from one or more of a Btk tyrosine kinase inhibitor, an Erbb2 tyrosine kinase receptor inhibitor; an Erbb4 tyrosine kinase receptor inhibitor, an mTOR inhibitor, a thymidylate synthase inhibitor, an EGFR tyrosine kinase receptor inhibitor, an epidermal growth factor antagonist, a Fyn tyrosine kinase inhibitor, a kit tyrosine kinase inhibitor, a Lyn tyrosine kinase inhibitor, a NK cell receptor modulator, a PDGF receptor antagonist, a PARP inhibitor, a poly ADP ribose polymerase inhibitor, a poly ADP ribose polymerase 1 inhibitor, a poly ADP ribose polymerase 2 inhibitor, a poly ADP ribose polymerase 3 inhibitor, a galactosyltransferase modulator, a dihydropyrimidine dehydrogenase inhibitor, an orotate phosphoribosyltransferase inhibitor, a telomerase modulator, a mucin 1 inhibitor, a mucin inhibitor, a secretin agonist, a TNF related apoptosis inducing ligand modulator, an IL-17 gene stimulator, an interleukin-17E ligand, a neurokinin receptor agonist, a cyclin G1 inhibitor, a checkpoint inhibitor, a PD-1 inhibitor, a PD-L1 inhibitor, a CTLA4 inhibitor, a topoisomerase I inhibitor, an Alk-5 protein kinase inhibitor, a connective tissue growth factor ligand inhibitor, a notch-2 receptor antagonist, a notch-3 receptor antagonist, a hyaluronidase stimulator, a MEK-1 protein kinase inhibitor; MEK-2 protein kinase inhibitor, a GM-CSF receptor modulator; TNF alpha ligand modulator, a mesothelin modulator, an asparaginase stimulator, a caspase-3 stimulator; caspase-9 stimulator, a PKN3 gene inhibitor, a hedgehog protein inhibitor; smoothened receptor antagonist, an AKT1 gene inhibitor, a DHFR inhibitor, a thymidine kinase stimulator, a CD29 modulator, a fibronectin modulator, an interleukin-2 ligand, a serine protease inhibitor, a D40LG gene stimulator; TNFSF9 gene stimulator, a 2-oxoglutarate dehydrogenase inhibitor, a TGF-beta type II receptor antagonist, an Erbb3 tyrosine kinase receptor inhibitor, a cholecystokinin CCK2 receptor antagonist, a Wilms tumor protein modulator, a Ras GTPase modulator, an histone deacetylase inhibitor, a cyclin-dependent kinase 4 inhibitor A modulator, an estrogen receptor beta modulator, a 4-1BB inhibitor, a 4-1BBL inhibitor, a PD-L2 inhibitor, a B7-H3 inhibitor, a B7-H4 inhibitor, a BTLA inhibitor, a HVEM inhibitor, aTIM3 inhibitor, a GAL9 inhibitor, a LAG3 inhibitor, a VISTA inhibitor, a KIR inhibitor, a 2B4 inhibitor, a CD160 inhibitor and a CD66e modulator. In one embodiment, the one or more additional therapeutic compounds is selected from one or more of bavituximab, IMM-101, CAP1-6D, Rexin-G, genistein, CVac, MM-D37K, PCI-27483, TG-01, mocetinostat, LOAd-703, CPI-613, upamostat, CRS-207, NovaCaps, trametinib, Atu-027, sonidegib, GRASPA, trabedersen, nastorazepide, Vaccell, oregovomab, istiratumab, refametinib, regorafenib, lapatinib, selumetinib, rucaparib, pelareorep, tarextumab, PEGylated hyaluronidase, varlitinib, aglatimagene besadenovec, GBS-01, GI-4000, WF-10, galunisertib, afatinib, RX-0201, FG-3019, pertuzumab, DCVax-Direct, selinexor, glufosfamide, virulizin, yttrium (90Y) clivatuzumab tetraxetan, brivudine, nimotuzumab, algenpantucel-L, tegafur+gimeracil+oteracil potassium+calcium folinate, olaparib, ibrutinib, pirarubicin, Rh-Apo2L, tertomotide, tegafur+gimeracil+oteracil potassium, tegafur+gimeracil+oteracil potassium, masitinib, Rexin-G, mitomycin, erlotinib, adriamycin, dexamethasone, vincristine, cyclophosphamide, fluorouracil, topotecan, taxol, interferons, platinum derivatives, taxane, paclitaxel, vinca alkaloids, vinblastine, anthracyclines, doxorubicin, epipodophyllotoxins, etoposide, cisplatin, rapamycin, methotrexate, actinomycin D, dolastatin 10, colchicine, emetine, trimetrexate, metoprine, cyclosporine, daunorubicin, teniposide, amphotericin, alkylating agents, chlorambucil, 5-fluorouracil, campthothecin, metronidazole, Gleevec, Avastin, Vectibix, abarelix, aldesleukin, alemtuzumab, alitretinoin, allopurinol, altretamine, amifostine, anastrozole, arsenic trioxide, asparaginase, azacitidine, AZD9291, BCG Live, bevacuzimab, fluorouracil, bexarotene, bleomycin, bortezomib, busulfan, calusterone, capecitabine, camptothecin, carboplatin, carmustine, celecoxib, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, estramustine, etoposide phosphate, etoposide, exemestane, filgrastim, floxuridine fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, rociletinib, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, zoledronic acid, pembrolizumab, nivolumab, IBI-308, mDX-400, BGB-108, MEDI-0680, SHR-1210, PF-06801591, PDR-001, GB-226, STI-1110, durvalumab, atezolizumab, avelumab, BMS-936559, ALN-PDL, TSR-042, KD-033, CA-170, STI-1014, FOLFIRINOX and KY-1003. In one embodiment, the one or more additional therapeutic compound is FOLFIRINOX. In one embodiment, the one or more additional therapeutic compounds are gemcitabine and paclitaxel. In one embodiment, the one or more additional therapeutic compounds are gemcitabine and nab-paclitaxel.

In some embodiments, the invention provides diagnostic markers to prognose the response to TREM-1 therapy. In some embodiments, the invention provides prognostic markers to prognose the response to TREM-1 therapy. It is not meant to limit the markers to those described herein.

Accordingly, the invention provides for a method of treating cancer in an individual in need thereof by administering to the individual a therapeutically effective amount of at least one modulator which affects myeloid cells by action on the TREM-1/DAP-12 signaling pathway together with a therapeutically effective amount of an anticancer vaccine, an anticancer immunotherapy agent, anti-cancer immunomodulatory agent, an additional anticancer therapeutic, radiation therapy, surgery or a combination thereof. The subject of the present invention includes any human subject who has been diagnosed with, has symptoms of, or is at risk of developing a cancer or a pre- or post-cancerous condition.

The invention relates to personalized combination-therapy treatments for cancer that involve targeting specific cancers by their tumor environment. More specifically, the invention provides a method for treating various cancers by using modulators of the TREM-1/DAP-12 pathway together with other cancer therapies and the use of such combinations in the treatment of cancer. In certain embodiments, these modulators may possess the antitumor activity. In some embodiments, these modulators may not possess the antitumor activity. In one embodiment, these modulators include peptide variants and compositions that are capable of binding TREM-1 and reducing or blocking TREM-1 activity (signaling and/or activation). In one embodiment these peptide variants and compositions modulate the TREM-1-mediated immunological responses beneficial for the treatment of cancer. In one embodiment, the peptides and compositions of the present invention modulate TREM-1/DAP-12 receptor complex expressed on monocytes, macrophages and neutrophils. In one embodiment, the peptides and compositions of the present invention modulate TREM-1/DAP-12 receptor complex expressed on tumor-associated macrophages. In one embodiment, the invention provides a method for predicting the efficacy of standalone or combination-therapy treatment that involve TREM-1-targeting therapies in various cancers by analyzing biological samples from cancer patients for the presence of myeloid cells and for the expression levels of TREM-1, CSF-1, CSF-1R, IL-6 and other markers. In one embodiment, the peptides and compositions of the invention are conjugated to an imaging probe. In one embodiment, the invention provides for detecting the TREM-1-expressing cells and tissues in an individual with cancer using imaging techniques and the peptides and compositions of the invention containing an imaging probe. In one embodiment, the peptides and compositions of the invention are used in combinations thereof. In one embodiment, the peptides and compositions of the invention are used in combinations with other anticancer therapeutic agents. In one embodiment, the present invention relates to the targeted treatment, prevention and/or detection of cancer including but not limited to lung cancer including non-small cell lung cancer (NSCLC), pancreatic cancer, breast cancer, liver cancer, multiple myeloma, melanoma, leukemia, bladder cancer, central nervous system (CNS) cancer, stomach cancer, prostate cancer, colorectal cancer, colon cancer, brain cancer, gastrointestinal cancer, gastric cancer, ovarian cancer, renal cancer, skin cancer, osteosarcoma, endometrial cancer, esophageal cancer, kidney cancer, thyroid cancer, neuroblastoma, neurofibroma, glioma, glioblastoma, glioblastoma multiforme, head and neck cancer, cervical cancer, pigmented villonodular synovitis (PVNS) and other cancers in which myeloid cells are involved or recruited and cancer cachexia.

In some embodiments, cancer is selected from the list including but not limited to lung cancer including NSCLC, pancreatic cancer, breast cancer, liver cancer, multiple myeloma, melanoma, leukemia, bladder cancer, central nervous system (CNS) cancer, stomach cancer, prostate cancer, colorectal cancer, colon cancer, brain cancer, gastrointestinal cancer, gastric cancer, ovarian cancer, renal cancer, skin cancer, osteosarcoma, endometrial cancer, esophageal cancer, kidney cancer, thyroid cancer, neuroblastoma, neurofibroma, glioma, glioblastoma, glioblastoma multiforme, head and neck cancer, cervical cancer, giant cell tumor of the tendon sheath (GCTTS), tenosynovial giant cell tumor (TGCT; also referred to in the art as TSGCT), PVNS and other cancers in which myeloid cells are involved or recruited and cancer cachexia.

In some embodiments, the modulators of the TREM-1/DAP-12 signaling pathway are capable of suppressing tumor growth in the subject. In another aspect, the modulators are capable of delaying the development of cancer in the subject. In another aspect, the modulators are capable of reducing tumor size in the subject. In another aspect, the modulators are capable of treating cancer in the subject. In another aspect, the modulators are capable of treating cancer in the subject. In another aspect, the modulators are capable of increasing survival of the subject.

In some embodiments, the modulators are capable of binding TREM-1 and reducing or blocking TREM-1 activity (signaling and/or activation). In some embodiments, the modulators comprise peptide variants and compositions that are capable of binding TREM-1 and reducing or blocking TREM-1 activity (signaling and/or activation) together with a pharmaceutically acceptable excipient, carrier, diluent, salt or a combination thereof. In some embodiments, the modulators comprise antibodies or fragments thereof that are capable of binding TREM-1 and reducing or blocking TREM-1 activity (signaling and/or activation) together with a pharmaceutically acceptable excipient, carrier, diluent, salt or a combination thereof.

The methods of combination therapy featured in the present invention may result in a synergistic effect, wherein the effect of a combination of compounds or other therapeutic agents is greater than the sum of the effects resulting from administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

In another aspect, the invention provides for a method of predicting the efficacy of TREM-1 targeted therapies in an individual with cancer by: (a) obtaining a biological sample from the individual; (b) determining the number of myeloid cells in the biological sample; (c) determining the expression levels of TREM-1 in the cells contained within the biological sample.

In another aspect, the invention provides for a method of detecting TREM-1 expression levels in an individual with cancer by: (a) administering to the individual the peptide variants and composition of the present invention having an affinity for TREM-1 and an imaging probe in a detectably effective amount; (b) imaging at least a portion of the patient; (c) detecting the labeled probe, wherein the location of the labeled probe corresponds to at least one symptom of the myeloid cell-related condition.

In certain embodiments, the invention provides for a diagnostic method of detecting TREM-1 expression levels in an individual with cancer by: (a) administering to the individual the modulators of TREM-1 transmembrane signaling having an affinity for TREM-1 and an imaging probe in a detectably effective amount; (b) imaging at least a portion of the patient; (c) detecting the labeled probe, wherein the location of the labeled probe corresponds to at least one symptom of the myeloid cell-related cancer condition and correlates with the TREM-1 expression levels and the higher the levels are, the better the patient is predicted to respond to a TREM-1 inhibitory therapy using the modulators of the TREM-1/DAP-12 signaling pathway as standalone therapy or in combinations with other anticancer treatments.

In some embodiments, the invention provides a method for treating cancer in a patient in need thereof by modulating immune system activity, said method comprising administering to said patient an amount of a TREM-1 inhibitor that is effective for inhibiting the TREM-1/DAP-12 signaling pathway and suppressing tumor growth, or a combination thereof. In one embodiment, said method further comprises administering the amount of the TREM-1 inhibitor together with a pharmaceutically acceptable excipient, carrier, diluents, or a combination thereof. In one embodiment, said method further comprises administering to said patient an anticancer vaccine, an anticancer immunotherapy agent, anti-cancer immunomodulatory agent, an additional anticancer therapeutic, radiation therapy or a combination thereof. In some embodiments, said anticancer vaccine is selected from the group consisting of Gardasil, Cervarix, and Sipuleucel-T/Provenge. In some embodiments, said anticancer immunotherapy agent is selected from the group consisting of Alemtuzumab, Ipilimumab, Nivolumab, Pembrolizumab, Rituximab, Interferon, Interleukin, and a combination thereof. In some embodiments, said anti-cancer immunomodulatory agent is selected from the group consisting of thalidomide, lenolidomide, pomalidomide, and a combination thereof. In some embodiments, said additional anti-cancer therapeutic is selected from the group consisting of an alkylating agent, a tubulin inhibitor, a topoisomerase inhibitor, proteasome inhibitor, a CHK1 inhibitor, a CHK2 inhibitor, PARP inhibitor, doxorubicin, epirubicin, vinblastine, etoposide, topotecan, bleomycin, and mytomycin c. In some embodiments, said alkylating agent is selected from the group consisting of Dacarbazine, Procarbazine, Carmustine, Lomustine, Uramustine, BuSulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin. In some embodiments, said tubulin inhibitor is selected from the group consisting of Taxol, Docetaxel, Vinblastin, Epothilone, Colchicine, Cryptophycin, BMS 347550, Rhizoxin, Ecteinascidin, Dolastin 10, Cryptophycin 52, and IDN-5109. In some embodiments, said topoisomerase inhibitor is a topoisomerase I inhibitor selected from the group consisting of Irinotecan, Topotecan, and Camptothecins (CPT). In some embodiments, said topoisomerase inhibitor is a topoisomerase II inhibitor selected from the group consisting of Amsacrine, Etoposide, Teniposide, Epipodophyllotoxins, and ellipticine. In some embodiments, said proteasome inhibitor is selected from the group consisting of Velcade (bortezomib), and Kyprolis (carfilzomib). In some embodiments, said CHK1 inhibitor is selected from the group consisting of TCS2312, PF-0047736, AZ07762, A-69002, and A-64 1397. In some embodiments, said PARP inhibitor is selected from the group consisting of Olaparib, ABT-888, (veliparib), KU-59436, AZD-2281, AG-014699, BSI-201, BGP-15, INO-1001, ONO-2231 and the like. In some embodiments, said radiation therapy is administered to said patient. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for detecting TREM-1/DAP-12 expression levels in a patient with cancer in need thereof, said method comprising administering to said patient an amount of a TREM-1 inhibitor that is conjugated to at least one imaging probe, or a combination thereof. In some embodiments, said imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for treating cancer in a patient in need thereof by modulating immune system activity, said method comprising administering to said patient an amount of a TREM-1 inhibitor that is effective for inhibiting the TREM-1/DAP-12 signaling pathway and suppressing tumor growth, or a combination thereof. In some embodiments, said TREM-1 inhibitor comprises a variant TREM-1 inhibitory peptide sequence derived from transmembrane domain sequences of human or animal TREM-1 and/or its signaling subunit, DAP-12, thereof. In some embodiments, said a variant TREM-1 inhibitory peptide sequence comprises amino acid sequence Gly-Phe-Leu-Ser-Lys-Ser-Leu-Val-Phe, wherein Gly is glycine, Phe is phenylalanine, Leu is leucine, Ser is serine, Lys is lysine, and Val is valine. In some embodiments, said variant TREM-1 inhibitory peptide sequence is conjugated to at least one unmodified or modified amphipathic peptide sequence. In some embodiments, said an unmodified or modified amphipathic peptide sequence is derived from amino acid sequences of apolipoproteins selected from the group consisting of A-I, A-II, A-IV, B, C-I, C-II, C-III, and E, and any combination thereof. In some embodiments, said a modified amphipathic peptide sequence derived from amino acid sequences of apolipoproteins selected from the group consisting of A-I, A-II, A-IV, B, C-I, C-II, C-III, and E, and any combination thereof contains at least one amino acid residue which is chemically or enzymatically modified. In some embodiments, said a chemically or enzymatically modified amino acid residue is oxidized, halogenated or nitrated. In some embodiments, said an oxidized amino acid residue is the methionine residue. In some embodiments, said an unmodified amphipathic peptide sequence is derived from an apolipoprotein A-I amino acid sequence and comprises amino acid sequence Pro-Tyr-Leu-Asp-Asp-Phe-Gln-Lys-Lys-Trp-Gln-Glu-Glu-Met-Glu-Leu-Tyr-Arg-Gln-Lys-Val-Glu, wherein Pro is proline, Tyr is tyrosine, Leu is leucine, Asp, asparagine, Phe is phenylalanine, Gln is glutamine, Lys is lysine, Trp is tryptophan, Glu is glutamic acid, Met is methionine, Arg is arginine, and Val is valine. In some embodiments, said an unmodified amphipathic peptide sequence is derived from an apolipoprotein A-I amino acid sequence and comprises amino acid sequence Pro-Leu-Gly-Glu-Glu-Met-Arg-Asp-Arg-Ala-Arg-Ala-His-Val-Asp-Ala-Leu-Arg-Thr-His-Leu-Ala, wherein Pro is proline, Leu is leucine, Gly is glycine, Glu is glutamic acid, Met is methionine, Arg is arginine, Asp, asparagine, Ala is alanine, His is histidine, Val is valine, and Thr is threonine. In some embodiments, said a modified amphipathic peptide sequence is derived from an apolipoprotein A-I amino acid sequence and comprises amino acid sequence Pro-Tyr-Leu-Asp-Asp-Phe-Gln-Lys-Lys-Trp-Gln-Glu-Glu-Met(O)-Glu-Leu-Tyr-Arg-Gln-Lys-Val-Glu, wherein Pro is proline, Tyr is tyrosine, Leu is leucine, Asp, asparagine, Phe is phenylalanine, Gln is glutamine, Lys is lysine, Trp is tryptophan, Glu is glutamic acid, Met(O) is methionine sulfoxide, Arg is arginine, and Val is valine. In some embodiments, said a modified amphipathic peptide sequence is derived from an apolipoprotein A-I amino acid sequence and comprises amino acid sequence Pro-Leu-Gly-Glu-Glu-Met(O)-Arg-Asp-Arg-Ala-Arg-Ala-His-Val-Asp-Ala-Leu-Arg-Thr-His-Leu-Ala, wherein Pro is proline, Leu is leucine, Gly is glycine, Glu is glutamic acid, Met is methionine sulfoxide, Arg is arginine, Asp, asparagine, Ala is alanine, His is histidine, Val is valine, and Thr is threonine. In some embodiments, said B is conjugated to an additional peptide sequence to enhance the targeting efficacy. In some embodiments, said an additional peptide sequence comprises amino acid sequence Arg-Gly-Asp (RGD), wherein Arg is arginine; Gly is glycine; and Asp is asparagine. In some embodiments, said A is conjugated to at least one additional therapeutic agent to enhance the therapeutic efficacy. In some embodiments, said an additional therapeutic agent is selected from the group of anticancer, antibacterial, antiviral, autoimmune, anti-inflammatory and cardiovascular agents, antioxidants, therapeutic peptides, and any combination thereof. In some embodiments, said anticancer therapeutic agent is selected from the group comprising paclitaxel, valrubicin, doxorubicin, taxotere, campotechin, etoposide, and any combination thereof. In some embodiments, said A and/or B are conjugated to at least one imaging probe. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol.

In some embodiments, the invention provides a method of making a synthetic lipopeptide nanoparticle, said method comprising: a) co-dissolving a predetermined amount of a mixture of neutral and/or charged lipids with: i. a predetermined amount of cholesterol; and ii. a predetermined amount of triglycerides and/or cholesteryl ester; b) drying the mixture of step (a) under nitrogen; c) co-dissolving the dried mixture of step (b) with: i. a predetermined amount of sodium cholate; and ii. a predetermined amount of the compound of claim 1; for a time period sufficient to allow the components to self-assemble into synthetic lipopeptide particles; d) removing sodium cholate from the mixture of step (c); and e) isolating particles that have a size of between about 5 to about 200 nm diameter. In some embodiments, said lipid is conjugated to at least one imaging probe. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol. In some embodiments, said lipid is selected from the group comprising cholesterol, a cholesteryl ester, a phospholipid, a glycolipid, a sphingolipid, a cationic lipid, a diacylglycerol, and a triacylglycerol. In some embodiments, said phospholipid is selected from the group comprising phosphatidylcholine (PC), phosphatidylethanolamine (PE), phosphatidylserine (PS), phosphatidylinositol (PI), phosphatidylglycerol (PG), cardiolipin (CL), sphingomyelin (SM), phosphatidic acid (PA), and any combination thereof. In some embodiments, said lipid is polyethylene glycol(PEG)ylated.

In some embodiments, the invention provides a method of imaging a myeloid cell-related condition, comprising a) providing; i) a patient having at least one symptom of a disease or condition in which myeloid cells are involved or recruited, and ii) a labeled probe, wherein the labeled probe includes the compositions of claims 1, 3, 4, and 21-25 having an affinity for TREM-1 and an imaging probe; b) administering said composition to said patient in a detectably effective amount c) imaging at least a portion of the patient; and d) detecting the labeled probe, wherein the location of the labeled probe corresponds to at least one symptom of the myeloid cell-related condition. In some embodiments, said a myeloid cell-related condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, inflammatory bowel disease, empyema, acute mesenteric ischemia, hemorrhagic shock, multiple sclerosis, autoimmune diseases (e.g., rheumatoid arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as interstitial pneumonitis and asthma, inflammatory bowel disease such as Crohn's disease, inflammatory arthritis retinopathy such as retinopathy of prematurity and diabetic retinopathy, Alzheimer's, Parkinson's and Huntington's diseases), transplant (e.g., heart/lung transplants) rejection reactions, and other diseases and conditions where myeloid cells are involved or recruited.

In some embodiments, the invention provides a method of treating a myeloid cell-related condition, comprising: a) providing; i) a patient having at least one symptom of a disease or condition in which myeloid cells are involved or recruited, and ii) the compositions of claims 1, 3, 4, and 23 capable of inhibiting TREM-1; b) administering said composition to said patient under conditions such that said at least one symptom is reduced. In some embodiments, said a myeloid cell-related condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, inflammatory bowel disease, empyema, acute mesenteric ischemia, hemorrhagic shock, multiple sclerosis, autoimmune diseases (e.g., rheumatoid arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as interstitial pneumonitis and asthma, inflammatory bowel disease such as Crohn's disease, inflammatory arthritis retinopathy such as retinopathy of prematurity and diabetic retinopathy, Alzheimer's, Parkinson's and Huntington's diseases), transplant (e.g., heart/lung transplants) rejection reactions, and other diseases and conditions where myeloid cells are involved or recruited.

In some embodiments, the invention provides a method of imaging a T cell-related condition, comprising a) providing; i) a patient having at least one symptom of a disease or condition in which T cells are involved or recruited, and ii) a labeled probe, wherein the labeled probe includes the compositions of claims 1, 5, 6, and 21-25 having an affinity for TCR and an imaging probe; b) administering said composition to said patient in a detectably effective amount c) imaging at least a portion of the patient; and d) detecting the labeled probe, wherein the location of the labeled probe corresponds to at least one symptom of the T cell-related condition. In some embodiments, said T cell-related condition is selected from the group including but not limited to include, but are not limited to, systemic lupus erythematosus, rheumatoid arthritis, multiple sclerosis, scleroderma, type I diabetes, gastroenterological conditions e.g. inflammatory bowel disease, Crohn's disease, celiac disease, Guillain-Barre syndrome, Hashimoto's disease, pernicious anaemia, primary biliary cirrhosis, chronic active hepatitis; skin problems e.g. atopic dermatitis, psoriasis, pemphigus vulgaris; cardiovascular problems e.g. autoimmune pericarditis, allergic diathesis e.g. delayed type hypersensitivity, contact dermatitis, AIDS virus, herpes simplex/zoster, respiratory conditions e.g. allergic alveolitis, inflammatory conditions e.g. myositis, ankylosing spondylitis, tissue/organ rejection, and other diseases and conditions where T cells are involved or recruited.

In some embodiments, the invention provides a method of treating a T cell-related condition, comprising: a) providing; i) a patient having at least one symptom of a disease or condition in which T cells are involved or recruited, and ii) the compositions of claims 1, 5, 6, and 23 capable of inhibiting TCR; b) administering said composition to said patient under conditions such that said at least one symptom is reduced. In some embodiments, said a T cell-related condition is selected from the group including but not limited to include, but are not limited to, systemic lupus erythematosus, rheumatoid arthritis, multiple sclerosis, scleroderma, type I diabetes, gastroenterological conditions e.g. inflammatory bowel disease, Crohn's disease, celiac disease, Guillain-Barre syndrome, Hashimoto's disease, pernicious anaemia, primary biliary cirrhosis, chronic active hepatitis; skin problems e.g. atopic dermatitis, psoriasis, pemphigus vulgaris; cardiovascular problems e.g. autoimmune pericarditis, allergic diathesis e.g. delayed type hypersensitivity, contact dermatitis, AIDS virus, herpes simplex/zoster, respiratory conditions e.g. allergic alveolitis, inflammatory conditions e.g. myositis, ankylosing spondylitis, tissue/organ rejection, and other diseases and conditions where T cells are involved or recruited.

In some embodiments, the invention provides a method of reducing pain in a subject with pigmented villonodular synovitis (PVNS) tumor, comprising administering to the subject an amount of a TREM-1 modulator that is effective for inhibiting the TREM-1/DAP-12 signaling pathway and capable of reducing pain in PVNS subjects independently of tumor response. In some embodiments, said PVNS tumor has a tumor volume. In some embodiments, said inhibition reduces said PVNS tumor volume by at least 30% after administration of at least two, at least three, at least four, at least five, at least six, at least seven, at least eight, at least nine, or at least ten doses of the modulator that inhibits the TREM-1/DAP-12 signaling pathway. In some embodiments, said tumor volume is tumor volume in a single joint. In some embodiments, said single joint is selected from a hip joint and a knee joint. In some embodiments, said tumor volume is total tumor volume in all joints affected by PVNS. In some embodiments, said modulator is an antibody. In some embodiments, prior to administering the first dose of said antibody, the subject received a prior therapy selected from surgical synovectomy, radiation beam therapy, radio isotope synovectomy, joint replacement and CSF1/CSF1R inhibitor. In some embodiments, said PVNS recurred or progressed after the prior therapy. In some embodiments, said antibody is administered prior to a therapy selected from surgical synovectomy, radiation beam therapy, radio isotope synovectomy, and joint replacement, or wherein the subject has a tumor that is unresectable. In some embodiments, said subject has not received prior treatment with a CSF1R inhibitor. In one embodiment, said method further comprises administering the amount of the TREM-1 modulator together with a pharmaceutically acceptable excipient, carrier, diluents, or a combination thereof. In one embodiment, said method further comprises administering the amount of the TREM-1 modulator together with an amount of an anticancer vaccine, an anticancer immunotherapy agent, anti-cancer immunomodulatory agent, an additional anticancer therapeutic, radiation therapy, or a combination thereof. In some embodiments, said anticancer vaccine is selected from the group consisting of Gardasil, Cervarix, and Sipuleucel-T/Provenge. In some embodiments, said anticancer immunotherapy agent is selected from the group consisting of Alemtuzumab, Ipilimumab, Nivolumab, Pembrolizumab, Rituximab, Interferon, Interleukin, and a combination thereof. In some embodiments, said anti-cancer immunomodulatory agent is selected from the group consisting of thalidomide, lenolidomide, pomalidomide, and a combination thereof. In some embodiments, said additional anti-cancer therapeutic is selected from the group consisting of an alkylating agent, a tubulin inhibitor, a topoisomerase inhibitor, proteasome inhibitor, a CHK1 inhibitor, a CHK2 inhibitor, PARP inhibitor, CSF1/CSF1R inhibitor, doxorubicin, epirubicin, vinblastine, etoposide, topotecan, bleomycin, and mytomycin c. In some embodiments, said alkylating agent is selected from the group consisting of Dacarbazine, Procarbazine, Carmustine, Lomustine, Uramustine, BuSulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin. In some embodiments, said tubulin inhibitor is selected from the group consisting of Taxol, Docetaxel, Vinblastin, Epothilone, Colchicine, Cryptophycin, BMS 347550, Rhizoxin, Ecteinascidin, Dolastin 10, Cryptophycin 52, and IDN-5109. In some embodiments, said topoisomerase inhibitor is a topoisomerase I inhibitor selected from the group consisting of Irinotecan, Topotecan, and Camptothecins (CPT). In some embodiments, said topoisomerase inhibitor is a topoisomerase II inhibitor selected from the group consisting of Amsacrine, Etoposide, Teniposide, Epipodophyllotoxins, and ellipticine. In some embodiments, said proteasome inhibitor is selected from the group consisting of Velcade (bortezomib), and Kyprolis (carfilzomib). In some embodiments, said CHK1 inhibitor is selected from the group consisting of TCS2312, PF-0047736, AZ07762, A-69002, and A-64 1397. In some embodiments, said PARP inhibitor is selected from the group consisting of Olaparib, ABT-888, (veliparib), KU-59436, AZD-2281, AG-014699, BSI-201, BGP-15, INO-1001, ONO-2231 and the like. In some embodiments, said CSF1/CSF1R inhibitor is selected from the group consisting of CSF1R kinase inhibitor, an antibody that binds CSF1R and the like. In some embodiments, said CSF1R kinase inhibitor is imatinib or nilotinib. In some embodiments, said CSF1R kinase inhibitor is PLX3397. In some embodiments, said anti-CSF1R antibody blocks binding of CSF1 and/or IL-34 to CSF1R. In some embodiments, said anti-CSF1R antibody inhibits ligand-induced CSF1R phosphorylation in vitro. In some embodiments, said antibody is a humanized antibody. In some embodiments, a radiation therapy is administered to said patient. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for detecting TREM-1/DAP-12 expression levels in a patient with cancer in need thereof, said method comprising administering to said patient an amount of a TREM-1 inhibitor that is conjugated to at least one imaging probe, or a combination thereof.

In some embodiments, the invention provides a method of predicting the efficacy of TREM-1 targeted therapies in an individual with the proliferative disorder by: (a) obtaining a biological sample from the individual; (b) determining the number of myeloid cells in the biological sample; (c) determining the expression levels of TREM-1 in the cells contained within the biological sample; (d) measuring the level of soluble form of the human TREM-1 receptor in the biological sample.

In some embodiments, the invention provides a method of diagnosing disease of the proliferative disorder in a subject, wherein said disease is PVNS or TGCT, which method comprises the steps of (a) measuring a level of the soluble form of the human TREM-1 receptor in a biological sample obtained from said subject; (b) comparing the measured level of the soluble form of the human TREM-1 receptor in the sample with a mean level in a control population of individuals not PVNS or TGCT; (c) correlating elevated levels of the soluble form of the human TREM-1 receptor with the presence or extent of said proliferative disease. In some embodiments, said step of measuring the level of the soluble form of the human TREM-1 receptor comprises the steps of: (a) contacting said biological sample with a compound capable of binding the soluble form of the human TREM-1 receptor; (b) detecting the level of the soluble form of the human TREM-1 receptor present in the sample by observing the level of binding between said compound and the soluble form of the human TREM-1 receptor. In one embodiment, said method further comprises comprising the steps of measuring the level of the soluble form of the human TREM-1 receptor in a second or further sample from said subject, the first and second or further samples being obtained at different times; and comparing the levels in the samples to indicate the progression or remission of the proliferative disease. In some embodiments, said sample is selected from the group consisting of whole blood, blood serum, blood, plasma, urine, bronchoalveolar lavage fluid and synovial liquid. In some embodiments, said sample is from synovial fluid. In some embodiments, said sample is from blood serum or blood plasma. In some embodiments, said sample is a human sample. In some embodiments, said compound specifically binds the soluble form of the human TREM-1 receptor. In some embodiments, said compound capable of binding the soluble form of the human TREM-1 receptor is an antibody raised against all or part of the TREM-1 receptor. In some embodiments, said level of soluble form of the human TREM-1 receptor is measured by an immunochemical technique. In one embodiment, said method further comprises an additional step of measuring the level of TREM-1-Ligand in one or more biological samples obtained from said subject. In some embodiments, said imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl201, K42, In111, Fe.59, Tc99m, Cr51, Ga67, Ga68, Cu64, Rb82, Mo99, Dy165, Fluorescein, Carboxyfluorescein, Calcein, F18, Xe133, I125, I131, I123, P32, C11, N13, O15, Br76, Kr81, Diatrizoate, Metrizoate, Isopaque, Ioxaglate, Iopamidol, Iohexol, Iodixanol. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for treating cancer in a patient in need thereof, said method comprising administering to said patient an amount of a TREM-1 inhibitor that is effective for inhibiting the TREM-1/DAP-12 signaling pathway and suppressing tumor growth and an amount of an anticancer vaccine, an anticancer immunotherapy agent, anti-cancer immunomodulatory agent, an additional anticancer therapeutic, radiation therapy, or a combination thereof. In one embodiment, said method further comprises administering the amount of the TREM-1 inhibitor together with a pharmaceutically acceptable excipient, carrier, diluents, or a combination thereof. In some embodiments, said anticancer vaccine is selected from the group consisting of Gardasil, Cervarix, and Sipuleucel-T/Provenge. In some embodiments, said anticancer immunotherapy agent is selected from the group consisting of Alemtuzumab, Ipilimumab, Nivolumab, Pembrolizumab, Rituximab, Interferon, Interleukin, and a combination thereof. In some embodiments, said anti-cancer immunomodulatory agent is selected from the group consisting of thalidomide, lenolidomide, pomalidomide, and a combination thereof. In some embodiments, said additional anti-cancer therapeutic is selected from the group consisting of an alkylating agent, a tubulin inhibitor, a topoisomerase inhibitor, proteasome inhibitor, a CHK1 inhibitor, a CHK2 inhibitor, PARP inhibitor, doxorubicin, epirubicin, vinblastine, etoposide, topotecan, bleomycin, and mytomycin c. In some embodiments, said alkylating agent is selected from the group consisting of Dacarbazine, Procarbazine, Carmustine, Lomustine, Uramustine, BuSulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin. In some embodiments, said tubulin inhibitor is selected from the group consisting of Taxol, Docetaxel, Vinblastin, Epothilone, Colchicine, Cryptophycin, BMS 347550, Rhizoxin, Ecteinascidin, Dolastin 10, Cryptophycin 52, and IDN-5109. In some embodiments, said topoisomerase inhibitor is a topoisomerase I inhibitor selected from the group consisting of Irinotecan, Topotecan, and Camptothecins (CPT). In some embodiments, said topoisomerase inhibitor is a topoisomerase II inhibitor selected from the group consisting of Amsacrine, Etoposide, Teniposide, Epipodophyllotoxins, and ellipticine. In some embodiments, said proteasome inhibitor is selected from the group consisting of Velcade (bortezomib), and Kyprolis (carfilzomib). In some embodiments, said CHK1 inhibitor is selected from the group consisting of TCS2312, PF-0047736, AZ07762, A-69002, and A-64 1397. In some embodiments, said PARP inhibitor is selected from the group consisting of Olaparib, ABT-888, (veliparib), KU-59436, AZD-2281, AG-014699, BSI-201, BGP-15, INO-1001, ONO-2231 and the like. In some embodiments, a radiation therapy is administered to said patient. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for detecting TREM-1/DAP-12 expression levels in a patient with cancer in need thereof, said method comprising administering to said patient an amount of a TREM-1 inhibitor that is conjugated to at least one imaging probe, or a combination thereof. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl201, K42, In111, Fe.59, Tc99m, Cr51, Ga67, Ga68, Cu64, Rb82, Mo99, Dy165, Fluorescein, Carboxyfluorescein, Calcein, F18, Xe133, I125, I131, I123, P32, C11, N13, O15, Br76, Kr81, Diatrizoate, Metrizoate, Isopaque, Ioxaglate, Iopamidol, Iohexol, Iodixanol. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for treating cancer in a patient in need thereof, said method comprising administering to said patient an amount of a TREM-1 inhibitor that is effective for inhibiting the TREM-1/DAP-12 signaling pathway and suppressing tumor growth and an amount of an anticancer vaccine, an anticancer immunotherapy agent, anti-cancer immunomodulatory agent, an additional anticancer therapeutic, radiation therapy, or a combination thereof. In one embodiment, said TREM-1 inhibitor comprises a variant TREM-1 inhibitory peptide sequence derived from transmembrane domain sequences of human or animal TREM-1 and/or its signaling subunit, DAP-12, thereof. In some embodiments, said a variant TREM-1 inhibitory peptide sequence comprises amino acid sequence Gly-Phe-Leu-Ser-Lys-Ser-Leu-Val-Phe, wherein Gly is glycine, Phe is phenylalanine, Leu is leucine, Ser is serine, Lys is lysine, and Val is valine. In some embodiments, said variant TREM-1 inhibitory peptide sequence is conjugated to at least one unmodified or modified amphipathic peptide sequence. In some embodiments, said an unmodified or modified amphipathic peptide sequence is derived from amino acid sequences of apolipoproteins selected from the group consisting of A-I, A-II, A-IV, B, C-I, C-II, C-III, and E, and any combination thereof. In some embodiments, said a modified amphipathic peptide sequence derived from amino acid sequences of apolipoproteins selected from the group consisting of A-I, A-II, A-IV, B, C-I, C-II, C-III, and E, and any combination thereof contains at least one amino acid residue which is chemically or enzymatically modified. In some embodiments, said a chemically or enzymatically modified amino acid residue is oxidized, halogenated or nitrated. In some embodiments, said an oxidized amino acid residue is the methionine residue. In some embodiments, said an unmodified amphipathic peptide sequence is derived from an apolipoprotein A-I amino acid sequence and comprises amino acid sequence Pro-Tyr-Leu-Asp-Asp-Phe-Gln-Lys-Lys-Trp-Gln-Glu-Glu-Met-Glu-Leu-Tyr-Arg-Gln-Lys-Val-Glu, wherein Pro is proline, Tyr is tyrosine, Leu is leucine, Asp, asparagine, Phe is phenylalanine, Gln is glutamine, Lys is lysine, Trp is tryptophan, Glu is glutamic acid, Met is methionine, Arg is arginine, and Val is valine. In some embodiments, said an unmodified amphipathic peptide sequence is derived from an apolipoprotein A-I amino acid sequence and comprises amino acid sequence Pro-Leu-Gly-Glu-Glu-Met-Arg-Asp-Arg-Ala-Arg-Ala-His-Val-Asp-Ala-Leu-Arg-Thr-His-Leu-Ala, wherein Pro is proline, Leu is leucine, Gly is glycine, Glu is glutamic acid, Met is methionine, Arg is arginine, Asp, asparagine, Ala is alanine, His is histidine, Val is valine, and Thr is threonine. In some embodiments, said a modified amphipathic peptide sequence is derived from an apolipoprotein A-I amino acid sequence and comprises amino acid sequence Pro-Tyr-Leu-Asp-Asp-Phe-Gln-Lys-Lys-Trp-Gln-Glu-Glu-Met(O)-Glu-Leu-Tyr-Arg-Gln-Lys-Val-Glu, wherein Pro is proline, Tyr is tyrosine, Leu is leucine, Asp, asparagine, Phe is phenylalanine, Gln is glutamine, Lys is lysine, Trp is tryptophan, Glu is glutamic acid, Met(O) is methionine sulfoxide, Arg is arginine, and Val is valine. In some embodiments, said a modified amphipathic peptide sequence is derived from an apolipoprotein A-I amino acid sequence and comprises amino acid sequence Pro-Leu-Gly-Glu-Glu-Met(O)-Arg-Asp-Arg-Ala-Arg-Ala-His-Val-Asp-Ala-Leu-Arg-Thr-His-Leu-Ala, wherein Pro is proline, Leu is leucine, Gly is glycine, Glu is glutamic acid, Met is methionine sulfoxide, Arg is arginine, Asp, asparagine, Ala is alanine, His is histidine, Val is valine, and Thr is threonine. In some embodiments, said B is conjugated to an additional peptide sequence to enhance the targeting efficacy. In some embodiments, said an additional peptide sequence comprises amino acid sequence Arg-Gly-Asp (RGD), wherein Arg is arginine; Gly is glycine; and Asp is asparagine. In some embodiments, said A is conjugated to at least one additional therapeutic agent to enhance the therapeutic efficacy. In some embodiments, said an additional therapeutic agent is selected from the group of anticancer, antibacterial, antiviral, autoimmune, anti-inflammatory and cardiovascular agents, antioxidants, therapeutic peptides, and any combination thereof. In some embodiments, said anticancer therapeutic agent is selected from the group comprising paclitaxel, valrubicin, doxorubicin, taxotere, campotechin, etoposide, and any combination thereof. In some embodiments, said A and/or B are conjugated to at least one imaging probe. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl201, K42, In111, Fe.59, Tc99m, Cr51, Ga67, Ga68, Cu64, Rb82, Mo99, Dy165, Fluorescein, Carboxyfluorescein, Calcein, F18, Xe133, I125, I131, I123, P32, C11, N13, O15, Br76, Kr81, Diatrizoate, Metrizoate, Isopaque, Ioxaglate, Iopamidol, Iohexol, Iodixanol.

In some embodiments, the invention provides a method of making a synthetic lipopeptide nanoparticle, said method comprising: a) co-dissolving a predetermined amount of a mixture of neutral and/or charged lipids with: i. a predetermined amount of cholesterol; and ii. a predetermined amount of triglycerides and/or cholesteryl ester; b) drying the mixture of step (a) under nitrogen; c) co-dissolving the dried mixture of step (b) with: i. a predetermined amount of sodium cholate; and ii. a predetermined amount of the compound of claim 1; for a time period sufficient to allow the components to self-assemble into synthetic lipopeptide particles; d) removing sodium cholate from the mixture of step (c); and e) isolating particles that have a size of between about 5 to about 200 nm diameter. In some embodiments, said lipid is conjugated to at least one imaging probe. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl201, K42, In111, Fe.59, Tc99m, Cr51, Ga67, Ga68, Cu64, Rb82, Mo99, Dy165, Fluorescein, Carboxyfluorescein, Calcein, F18, Xe133, I125, I131, I123, P32, C11, N13, O15, Br76, Kr81, Diatrizoate, Metrizoate, Isopaque, Ioxaglate, Iopamidol, Iohexol, Iodixanol. In some embodiments, said lipid is selected from the group comprising cholesterol, a cholesteryl ester, a phospholipid, a glycolipid, a sphingolipid, a cationic lipid, a diacylglycerol, and a triacylglycerol. In some embodiments, said phospholipid is selected from the group comprising phosphatidylcholine (PC), phosphatidylethanolamine (PE), phosphatidylserine (PS), phosphatidylinositol (PI), phosphatidylglycerol (PG), cardiolipin (CL), sphingomyelin (SM), phosphatidic acid (PA), and any combination thereof. In some embodiments, said lipid is polyethylene glycol(PEG)ylated.

In some embodiments, the invention provides a method of imaging a myeloid cell-related condition, comprising a) providing; i) a patient having at least one symptom of a disease or condition in which myeloid cells are involved or recruited, and ii) a labeled probe, wherein the labeled probe includes the compositions of claims 1, 3, 4, and 21-25 having an affinity for TREM-1 and an imaging probe; b) administering said composition to said patient in a detectably effective amount c) imaging at least a portion of the patient; and d) detecting the labeled probe, wherein the location of the labeled probe corresponds to at least one symptom of the myeloid cell-related condition. In some embodiments, said a myeloid cell-related condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, inflammatory bowel disease, empyema, acute mesenteric ischemia, hemorrhagic shock, multiple sclerosis, autoimmune diseases (e.g., rheumatoid arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as interstitial pneumonitis and asthma, inflammatory bowel disease such as Crohn's disease, inflammatory arthritis retinopathy such as retinopathy of prematurity and diabetic retinopathy, Alzheimer's, Parkinson's and Huntington's diseases), transplant (e.g., heart/lung transplants) rejection reactions, and other diseases and conditions where myeloid cells are involved or recruited.

In some embodiments, the invention provides a method of treating a myeloid cell-related condition, comprising: a) providing; i) a patient having at least one symptom of a disease or condition in which myeloid cells are involved or recruited, and ii) the compositions of claims 1, 3, 4, and 23 capable of inhibiting TREM-1; b) administering said composition to said patient under conditions such that said at least one symptom is reduced. In some embodiments, said a myeloid cell-related condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, inflammatory bowel disease, empyema, acute mesenteric ischemia, hemorrhagic shock, multiple sclerosis, autoimmune diseases (e.g., rheumatoid arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as interstitial pneumonitis and asthma, inflammatory bowel disease such as Crohn's disease, inflammatory arthritis retinopathy such as retinopathy of prematurity and diabetic retinopathy, Alzheimer's, Parkinson's and Huntington's diseases), transplant (e.g., heart/lung transplants) rejection reactions, and other diseases and conditions where myeloid cells are involved or recruited.

In some embodiments, the invention provides a method of imaging a T cell-related condition, comprising a) providing; i) a patient having at least one symptom of a disease or condition in which T cells are involved or recruited, and ii) a labeled probe, wherein the labeled probe includes the compositions of claims 1, 5, 6, and 21-25 having an affinity for TCR and an imaging probe; b) administering said composition to said patient in a detectably effective amount c) imaging at least a portion of the patient; and d) detecting the labeled probe, wherein the location of the labeled probe corresponds to at least one symptom of the T cell-related condition.

In some embodiments, the invention provides a method of treating a T cell-related condition, comprising: a) providing; i) a patient having at least one symptom of a disease or condition in which T cells are involved or recruited, and ii) the compositions of claims 1, 5, 6, and 23 capable of inhibiting TCR; b) administering said composition to said patient under conditions such that said at least one symptom is reduced. In some embodiments, said a T cell-related condition is selected from the group including but not limited to include, but are not limited to, systemic lupus erythematosus, rheumatoid arthritis, multiple sclerosis, scleroderma, type I diabetes, gastroenterological conditions e.g. inflammatory bowel disease, Crohn's disease, celiac disease, Guillain-Barre syndrome, Hashimoto's disease, pernicious anaemia, primary biliary cirrhosis, chronic active hepatitis; skin problems e.g. atopic dermatitis, psoriasis, pemphigus vulgaris; cardiovascular problems e.g. autoimmune pericarditis, allergic diathesis e.g. delayed type hypersensitivity, contact dermatitis, AIDS virus, herpes simplex/zoster, respiratory conditions e.g. allergic alveolitis, inflammatory conditions e.g. myositis, ankylosing spondylitis, tissue/organ rejection, and other diseases and conditions where T cells are involved or recruited.

In another aspect, the invention provides for a method of predicting response of the subject to the treatment by using the modulators of TREM-1/DAP-12 signaling pathway in standalone or combination-therapy regimen by: (a) obtaining a biological sample from the subject; (b) determining the expression of CSF-1, CSF-1R, IL-6, TREM-1 and/or number of CD68-positive TAMs or a combination thereof, wherein the higher is the expression of CSF-1, CSF-1R, IL-6, TREM-1 or the higher is number of CD68-positive TAMs or a combination thereof, the better the patient is predicted to respond to a therapy that involves the modulators.

In some embodiments, the invention provides for a method of diagnosing cancer in which myeloid cells are involved or recruited in the subject and/or predicting response of the subject to the treatment by using the modulators of TREM-1/DAP-12 signaling pathway in standalone or combination-therapy regimen by: (a) administering to said patient an amount of at least one modulator capable of binding TREM-1 that is conjugated to at least one imaging probe, or a combination thereof, in a detectably effective amount; (b) imaging at least a portion of the patient; (c) detecting the labeled probe, wherein the location and amount of the labeled probe corresponds to at least one symptom of the myeloid cell-related cancer condition and the TREM-1 expression levels and the higher the expression level is, the better the patient is predicted to respond to a therapy that involves the modulators.

The invention relates to personalized medical treatments for scleroderma (systemic sclerosis, SSc). More specifically, the invention provides for treatment of scleroderma or a related autoimmune or a fibrotic condition by using modulators of the TREM-1/DAP-12 pathway standalone or together with other antifibrotic therapies and the use of such combinations in the treatment of scleroderma. In certain embodiments, these modulators may possess the antifibrotic activity. In some embodiments, these modulators may not possess the antifibrotic activity. In certain embodiments, these modulators may possess the anti-inflammatory activity. In one embodiment, these modulators include peptide variants and compositions that are capable of binding TREM-1 and reducing or blocking TREM-1 activity (signaling and/or activation). In one embodiment these peptide variants and compositions modulate the TREM-1-mediated immunological responses beneficial for the treatment of scleroderma or a related autoimmune or a fibrotic condition. In one embodiment, the peptides and compositions of the present invention modulate TREM-1/DAP-12 receptor complex expressed on monocytes, macrophages and neutrophils. In one embodiment, the peptides and compositions of the present invention modulate TREM-1/DAP-12 receptor complex expressed on SSc-associated macrophages. In one embodiment, the invention provides a method for predicting the efficacy of standalone or combination-therapy treatment that involve TREM-1-targeting therapies in scleroderma by analyzing biological samples from cancer patients for the presence of myeloid cells and for the expression levels of TREM-1, CSF-1, CSF-1R, IL-6 and other markers. In one embodiment, the peptides and compositions of the invention are conjugated to an imaging probe. In one embodiment, the invention provides for detecting the TREM-1-expressing cells and tissues in an individual with scleroderma using imaging techniques and the peptides and compositions of the invention containing an imaging probe. In one embodiment, the peptides and compositions of the invention are used in combinations thereof. In one embodiment, the peptides and compositions of the invention are used in combinations with other antifibrotic therapeutic agents. In one embodiment, the present invention relates to the targeted treatment, prevention and/or detection of scleroderma including but not limited to calcinosis, Raynaud's phenomenon, esophageal dysmotility, scleroderma, or telangiectasia syndrome (CREST).

The invention provides for a method of treating scleroderma (SSc) or a related autoimmune or a fibrotic condition in an individual in need thereof by administering to the individual an effective amount of an inhibitor of the TREM-1/DAP-12 pathway. In one aspect, the inhibitors are selected from peptide variants and compositions that suppress tumor growth by modulating the TREM-1/DAP-12 signaling pathway. In one embodiment, any or both the domains comprise minimal biologically active amino acid sequence. In one embodiment, the peptide variant comprises a cyclic peptide sequence. In one embodiment, the peptide variant comprises a disulfide-linked dimer. In one embodiment, the peptide variant includes amino acids selected from the group of natural and unnatural amino acids including, but not limited to, L-amino acids, or D-amino acids. In one embodiment, an imaging probe and/or an additional therapeutic agent is conjugated to the peptide variants and compositions of the invention. In one embodiment, the imaging agent is a Gd-based contrast agent (GBCA) for magnetic resonance imaging (MRI). In one embodiment, the imaging agent is a [⁶⁴Cu]-containing imaging probe for imaging systems such as a positron emission tomography (PET) imaging systems (and combined PET/computer tomography (CT) and PET/MRI systems). In one embodiment, the peptides and compositions of the invention are used in combinations thereof. In one embodiment, the peptides and compositions of the invention are used in combinations with other antifibrotic therapeutic agents. In certain embodiments, the peptide variants and compositions of the present invention are incorporated into long half-life synthetic lipopeptide particles (SLP). In certain embodiments, the peptide variants and compositions of the invention may incorporate into lipopeptide particles (LP) in vivo upon administration to the individual. In certain embodiments, the peptides and compositions of the invention can cross the blood-brain barrier (BBB), blood-retinal barrier (BRB) and blood-tumor barrier (BTB). Thus, in one aspect, the invention provides for a method for suppressing tumor growth in an individual in need thereof by administering to the individual an amount of a TREM-1 inhibitor that is effective for suppressing inflammation and fibrosis.

Some aspects of the invention provide methods for treating scleroderma or related autoimmune or a fibrotic condition in a subject by administering a therapeutically effective amount of a TREM-1 inhibitor to the subject in need of such a treatment. In some embodiments, scleroderma is a systemic sclerosis, which is a systemic autoimmune disease or systemic connective tissue disease. SSc is often characterized by deposition of collagen in the skin. In some cases, SSc involves deposition of collagen in organs, such as the kidneys, heart, lungs and/or stomach.

In other embodiments, scleroderma is a diffuse scleroderma. Diffuse scleroderma typically affects the skin and organs such as the heart, lungs, gastrointestinal tract, and kidneys. Still in other embodiments, scleroderma is a limited scleroderma that affects primarily the skin including, but not limited to, that of the face, neck and distal elbows and knees. Still in other embodiments, scleroderma is a limited scleroderma. In some instances, the limited scleroderma includes clinical conditions that affect the hands, arms, and face. In other instances, clinical conditions associated with the limited scleroderma include, calcinosis, Raynaud's phenomenon, esophageal dysfunction, sclerodactyl), telangiectasias and pulmonary arterial hypertension. Yet in other instances, scleroderma is a localized scleroderma.

In another aspect, the invention provides for a method of predicting the efficacy of TREM-1 targeted therapies in an individual with scleroderma by: (a) obtaining a biological sample from the individual; (b) determining the number of myeloid cells in the biological sample; (c) determining the expression levels of TREM-1 in the cells contained within the biological sample.

In another aspect, the invention provides for a method of detecting TREM-1 expression levels in an individual with scleroderma by: (a) administering to the individual the peptide variants and composition of the present invention having an affinity for TREM-1 and an imaging probe in a detectably effective amount; (b) imaging at least a portion of the patient; (c) detecting the labeled probe, wherein the location of the labeled probe corresponds to at least one symptom of the myeloid cell-related condition.

The present invention provides the compounds and compositions for TREM-1-targeted treatment of SSc and the methods for predicting the efficacy of these compositions. The invention further provides a method of using these compounds and compositions. These and other objects and advantages of the invention, as well as additional inventive features, will be apparent from the description of the invention provided herein.

In some embodiments, the invention provides a method for treating cancer in a patient in need thereof by modulating immune system activity, said method comprising administering to said patient an amount of a TREM-1 inhibitor that is effective for inhibiting the TREM-1/DAP-12 signaling pathway and suppressing tumor growth, or a combination thereof. In one embodiment, said method further comprises administering the amount of the TREM-1 inhibitor together with a pharmaceutically acceptable excipient, carrier, diluents, or a combination thereof. In one embodiment, said method further comprises administering to said patient an anticancer vaccine, an anticancer immunotherapy agent, anti-cancer immunomodulatory agent, an additional anticancer therapeutic, radiation therapy or a combination thereof. In some embodiments, said anticancer vaccine is selected from the group consisting of Gardasil, Cervarix, and Sipuleucel-T/Provenge. In some embodiments, said anticancer immunotherapy agent is selected from the group consisting of Alemtuzumab, Ipilimumab, Nivolumab, Pembrolizumab, Rituximab, Interferon, Interleukin, and a combination thereof. In some embodiments, said anti-cancer immunomodulatory agent is selected from the group consisting of thalidomide, lenolidomide, pomalidomide, and a combination thereof. In some embodiments, said additional anti-cancer therapeutic is selected from the group consisting of an alkylating agent, a tubulin inhibitor, a topoisomerase inhibitor, proteasome inhibitor, a CHK1 inhibitor, a CHK2 inhibitor, PARP inhibitor, doxorubicin, epirubicin, vinblastine, etoposide, topotecan, bleomycin, and mytomycin c. In some embodiments, said alkylating agent is selected from the group consisting of Dacarbazine, Procarbazine, Carmustine, Lomustine, Uramustine, BuSulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin. In some embodiments, said tubulin inhibitor is selected from the group consisting of Taxol, Docetaxel, Vinblastin, Epothilone, Colchicine, Cryptophycin, BMS 347550, Rhizoxin, Ecteinascidin, Dolastin 10, Cryptophycin 52, and IDN-5109. In some embodiments, said topoisomerase inhibitor is a topoisomerase I inhibitor selected from the group consisting of Irinotecan, Topotecan, and Camptothecins (CPT). In some embodiments, said topoisomerase inhibitor is a topoisomerase II inhibitor selected from the group consisting of Amsacrine, Etoposide, Teniposide, Epipodophyllotoxins, and ellipticine. In some embodiments, said proteasome inhibitor is selected from the group consisting of Velcade (bortezomib), and Kyprolis (carfilzomib). In some embodiments, said CHK1 inhibitor is selected from the group consisting of TCS2312, PF-0047736, AZ07762, A-69002, and A-64 1397. In some embodiments, said PARP inhibitor is selected from the group consisting of Olaparib, ABT-888, (veliparib), KU-59436, AZD-2281, AG-014699, BSI-201, BGP-15, INO-1001, ONO-2231 and the like. In one embodiment, said method further comprises a radiation therapy administered to said patient. In some embodiments, said at least one said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for detecting TREM-1/DAP-12 expression levels in a patient with cancer in need thereof, said method comprising administering to said patient an amount of a TREM-1 inhibitor that is conjugated to at least one imaging probe, or a combination thereof. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol. In some embodiments, said TREM-1 inhibitor comprises a variant TREM-1 inhibitory peptide sequence derived from transmembrane domain sequences of human or animal TREM-1 and/or its signaling subunit, DAP-12, thereof. In some embodiments, said at least one said TREM-1 inhibitor comprises LR12 and/or LP17 peptide variants and the like.

In some embodiments, the invention provides a method for treating cancer in a patient in need thereof by modulating immune system activity, said method comprising administering to said patient an amount of a TREM-1 inhibitor that is effective for inhibiting the TREM-1/DAP-12 signaling pathway and suppressing tumor growth, or a combination thereof. In some embodiments, said TREM-1 inhibitor comprises a variant TREM-1 inhibitory peptide sequence derived from transmembrane domain sequences of human or animal TREM-1 and/or its signaling subunit, DAP-12, thereof. In some embodiments, said variant TREM-1 inhibitory peptide sequence comprises amino acid sequence Gly-Phe-Leu-Ser-Lys-Ser-Leu-Val-Phe, wherein Gly is glycine, Phe is phenylalanine, Leu is leucine, Ser is serine, Lys is lysine, and Val is valine. In some embodiments, said variant TREM-1 inhibitory peptide sequence is conjugated to at least one unmodified or modified amphipathic peptide sequence. In some embodiments, said unmodified or modified amphipathic peptide sequence is derived from amino acid sequences of apolipoproteins selected from the group consisting of A-I, A-II, A-IV, B, C-I, C-II, C-III, and E, and any combination thereof. In some embodiments, said modified amphipathic peptide sequence derived from amino acid sequences of apolipoproteins selected from the group consisting of A-I, A-II, A-IV, B, C-I, C-II, C-III, and E, and any combination thereof contains at least one amino acid residue which is chemically or enzymatically modified. In some embodiments, said chemically or enzymatically modified amino acid residue is oxidized, halogenated or nitrated. In some embodiments, said oxidized amino acid residue is the methionine residue. In some embodiments, said unmodified amphipathic peptide sequence is derived from an apolipoprotein A-I amino acid sequence and comprises amino acid sequence Pro-Tyr-Leu-Asp-Asp-Phe-Gln-Lys-Lys-Trp-Gln-Glu-Glu-Met-Glu-Leu-Tyr-Arg-Gln-Lys-Val-Glu, wherein Pro is proline, Tyr is tyrosine, Leu is leucine, Asp, asparagine, Phe is phenylalanine, Gln is glutamine, Lys is lysine, Trp is tryptophan, Glu is glutamic acid, Met is methionine, Arg is arginine, and Val is valine. In some embodiments, said unmodified amphipathic peptide sequence is derived from an apolipoprotein A-I amino acid sequence and comprises amino acid sequence Pro-Leu-Gly-Glu-Glu-Met-Arg-Asp-Arg-Ala-Arg-Ala-His-Val-Asp-Ala-Leu-Arg-Thr-His-Leu-Ala, wherein Pro is proline, Leu is leucine, Gly is glycine, Glu is glutamic acid, Met is methionine, Arg is arginine, Asp, asparagine, Ala is alanine, His is histidine, Val is valine, and Thr is threonine. In some embodiments, said modified amphipathic peptide sequence is derived from an apolipoprotein A-I amino acid sequence and comprises amino acid sequence Pro-Tyr-Leu-Asp-Asp-Phe-Gln-Lys-Lys-Trp-Gln-Glu-Glu-Met(O)-Glu-Leu-Tyr-Arg-Gln-Lys-Val-Glu, wherein Pro is proline, Tyr is tyrosine, Leu is leucine, Asp, asparagine, Phe is phenylalanine, Gln is glutamine, Lys is lysine, Trp is tryptophan, Glu is glutamic acid, Met(O) is methionine sulfoxide, Arg is arginine, and Val is valine. In some embodiments, said modified amphipathic peptide sequence is derived from an apolipoprotein A-I amino acid sequence and comprises amino acid sequence Pro-Leu-Gly-Glu-Glu-Met(O)-Arg-Asp-Arg-Ala-Arg-Ala-His-Val-Asp-Ala-Leu-Arg-Thr-His-Leu-Ala, wherein Pro is proline, Leu is leucine, Gly is glycine, Glu is glutamic acid, Met is methionine sulfoxide, Arg is arginine, Asp, asparagine, Ala is alanine, His is histidine, Val is valine, and Thr is threonine. In some embodiments, said B is conjugated to an additional peptide sequence to enhance the targeting efficacy. In some embodiments, said an additional peptide sequence comprises amino acid sequence Arg-Gly-Asp (RGD), wherein Arg is arginine; Gly is glycine; and Asp is asparagine. In some embodiments, said A is conjugated to at least one additional therapeutic agent to enhance the therapeutic efficacy. In some embodiments, said an additional therapeutic agent is selected from the group of anticancer, antibacterial, antiviral, autoimmune, anti-inflammatory and cardiovascular agents, antioxidants, therapeutic peptides, and any combination thereof. In some embodiments, said anticancer therapeutic agent is selected from the group comprising paclitaxel, valrubicin, doxorubicin, taxotere, campotechin, etoposide, and any combination thereof. In some embodiments, said A and/or B are conjugated to at least one imaging probe. In some embodiments, said an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol.

In some embodiments, the invention provides a method of making a synthetic lipopeptide nanoparticle, said method comprising: a) co-dissolving a predetermined amount of a mixture of neutral and/or charged lipids with: i. a predetermined amount of cholesterol; and ii. a predetermined amount of triglycerides and/or cholesteryl ester; b) drying the mixture of step (a) under nitrogen; c) co-dissolving the dried mixture of step (b) with: i. a predetermined amount of sodium cholate; and ii. a predetermined amount of the compound of claim 1; for a time period sufficient to allow the components to self-assemble into synthetic lipopeptide particles; d) removing sodium cholate from the mixture of step (c); and e) isolating particles that have a size of between about 5 to about 200 nm diameter. In some embodiments, said lipid is conjugated to at least one imaging probe. In some embodiments, said imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵, Ga⁶⁷, Ga⁶⁸, Cu⁶⁴, Rb⁸², Mo⁹⁹, Dy¹⁶⁵ Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol. In some embodiments, said lipid is selected from the group comprising cholesterol, a cholesteryl ester, a phospholipid, a glycolipid, a sphingolipid, a cationic lipid, a diacylglycerol, and a triacylglycerol. In some embodiments, said phospholipid is selected from the group comprising phosphatidylcholine (PC), phosphatidylethanolamine (PE), phosphatidylserine (PS), phosphatidylinositol (PI), phosphatidylglycerol (PG), cardiolipin (CL), sphingomyelin (SM), phosphatidic acid (PA), and any combination thereof. In some embodiments, said lipid is polyethylene glycol(PEG)ylated.

In some embodiments, the invention provides a method of imaging a myeloid cell-related condition, comprising a) providing; i) a patient having at least one symptom of a disease or condition in which myeloid cells are involved or recruited, and ii) a labeled probe, wherein the labeled probe includes the compositions of claims 1, 3, 4, and 21-25 having an affinity for TREM-1 and an imaging probe; b) administering said composition to said patient in a detectably effective amount c) imaging at least a portion of the patient; and d) detecting the labeled probe, wherein the location of the labeled probe corresponds to at least one symptom of the myeloid cell-related condition. In some embodiments, said myeloid cell-related condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, inflammatory bowel disease, empyema, acute mesenteric ischemia, hemorrhagic shock, multiple sclerosis, autoimmune diseases (e.g., rheumatoid arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as interstitial pneumonitis and asthma, inflammatory bowel disease such as Crohn's disease, inflammatory arthritis retinopathy such as retinopathy of prematurity and diabetic retinopathy, Alzheimer's, Parkinson's and Huntington's diseases), transplant (e.g., heart/lung transplants) rejection reactions, and other diseases and conditions where myeloid cells are involved or recruited.

In some embodiments, the invention provides a method of treating a myeloid cell-related condition, comprising: a) providing; i) a patient having at least one symptom of a disease or condition in which myeloid cells are involved or recruited, and ii) the compositions of claims 1, 3, 4, and 23 capable of inhibiting TREM-1; b) administering said composition to said patient under conditions such that said at least one symptom is reduced. In some embodiments, said myeloid cell-related condition is selected from the group comprising cancer including but not limited to lung, pancreatic, breast, stomach, prostate, colon, brain and skin cancers, cancer cachexia, heart disease, atherosclerosis, peripheral artery disease, restenosis, stroke, bacterial infectious diseases, acquired immune deficiency syndrome (AIDS), allergic diseases, acute radiation syndrome, inflammatory bowel disease, empyema, acute mesenteric ischemia, hemorrhagic shock, multiple sclerosis, autoimmune diseases (e.g., rheumatoid arthritis, Sjogrens, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as interstitial pneumonitis and asthma, inflammatory bowel disease such as Crohn's disease, inflammatory arthritis retinopathy such as retinopathy of prematurity and diabetic retinopathy, Alzheimer's, Parkinson's and Huntington's diseases), transplant (e.g., heart/lung transplants) rejection reactions, and other diseases and conditions where myeloid cells are involved or recruited.

In some embodiments, the invention provides a method of imaging a T cell-related condition, comprising a) providing; i) a patient having at least one symptom of a disease or condition in which T cells are involved or recruited, and ii) a labeled probe, wherein the labeled probe includes the compositions of claims 1, 5, 6, and 21-25 having an affinity for TCR and an imaging probe; b) administering said composition to said patient in a detectably effective amount c) imaging at least a portion of the patient; and d) detecting the labeled probe, wherein the location of the labeled probe corresponds to at least one symptom of the T cell-related condition. In some embodiments, said T cell-related condition is selected from the group including but not limited to include, but are not limited to, systemic lupus erythematosus, rheumatoid arthritis, multiple sclerosis, scleroderma, type I diabetes, gastroenterological conditions e.g. inflammatory bowel disease, Crohn's disease, celiac disease, Guillain-Barre syndrome, Hashimoto's disease, pernicious anaemia, primary biliary cirrhosis, chronic active hepatitis; skin problems e.g. atopic dermatitis, psoriasis, pemphigus vulgaris; cardiovascular problems e.g. autoimmune pericarditis, allergic diathesis e.g. delayed type hypersensitivity, contact dermatitis, AIDS virus, herpes simplex/zoster, respiratory conditions e.g. allergic alveolitis, inflammatory conditions e.g. myositis, ankylosing spondylitis, tissue/organ rejection, and other diseases and conditions where T cells are involved or recruited.

In some embodiments, the invention provides a method of treating a T cell-related condition, comprising: a) providing; i) a patient having at least one symptom of a disease or condition in which T cells are involved or recruited, and ii) the compositions of claims 1, 5, 6, and 23 capable of inhibiting TCR; b) administering said composition to said patient under conditions such that said at least one symptom is reduced. In some embodiments, said T cell-related condition is selected from the group including but not limited to include, but are not limited to, systemic lupus erythematosus, rheumatoid arthritis, multiple sclerosis, scleroderma, type I diabetes, gastroenterological conditions e.g. inflammatory bowel disease, Crohn's disease, celiac disease, Guillain-Barre syndrome, Hashimoto's disease, pernicious anaemia, primary biliary cirrhosis, chronic active hepatitis; skin problems e.g. atopic dermatitis, psoriasis, pemphigus vulgaris; cardiovascular problems e.g. autoimmune pericarditis, allergic diathesis e.g. delayed type hypersensitivity, contact dermatitis, AIDS virus, herpes simplex/zoster, respiratory conditions e.g. allergic alveolitis, inflammatory conditions e.g. myositis, ankylosing spondylitis, tissue/organ rejection, and other diseases and conditions where T cells are involved or recruited.

The details of one or more embodiments of the invention are set forth in the accompanying Figures (Drawings) and Detailed Description of The Invention, as described herein and below. Other features, objects, and advantages of the invention will be apparent from the summary, description, figures and from the claims.

### BRIEF DESCRIPTION OF THE DRAWINGS

The following figures form part of the present specification and are included to further illustrate embodiments of the present invention. The invention may be better understood by reference to the figures in combination with the detailed description of the specific embodiments presented herein.

The patent or application file contains at least one drawing executed in color. Copies of this patent or patent application publication with color drawing(s) will be provided by the Office upon request and payment of the necessary fee.

FIG. 1 presents an exemplary schematic representation of one embodiment of a trifunctional peptide of the present invention comprising amino acid domains A and B where amino acid domain A represents a therapeutic peptide sequence with or without an attached drug compound and/or imaging probe that functions to treat, prevent and/or detect a disease or condition, whereas amino acid domain B represents an amphipathic alpha helical peptide sequence, with or without an additional targeting peptide sequence, and functions to 1) assist in the self-assembly of synthetic lipoprotein/lipopeptide nanoparticles (SLP) upon interaction with lipids or lipid mixtures in vitro, for use in transporting these trifunctional peptides as lipoprotein nanoparticles to sites of interest in vitro or in vivo and/or 2) form long half-life lipopeptide/lipoprotein particles upon interaction with endogenous lipoproteins for transporting these trifunctional peptides to the sites of interest. Endogenous lipoproteins may be lipoproteins added to or found in cell cultures, or lipoproteins in a mammalian body.

FIG. 2 presents schematic representations of embodiments of a TREM-1-related trifunctional peptide (TREM-1/TRIOPEP). GE31 (GFLSKSLVFPYLDDFQKKWQEEM(O)ELYRQKVE, M(O), methionine sulfoxide) comprises amino acid domain A and B (GFLSKSLVFPYLDDFQKKWQEEM(O)ELYRQKVE) where domain A represents a 9 amino acids-long human TREM-1 inhibitory therapeutic SCHOOL peptide sequence and functions to treat and/or prevent a TREM-1-related disease or condition, whereas domain B represents a 22 amino acids-long human apolipoprotein A-I helix 4 peptide sequence with a sulfoxidized methionine residue and functions to assist in the self-assembly of synthetic lipopeptide particles (SLP) in vitro for targeting the particles to myeloid cells (e.g. macrophages). GA31 (GFLSKSLVFPLGEEM(O)RDRARAHVDALRTHLA, M(O), methionine sulfoxide).

Abbreviations: apo, apolipoprotein; SCHOOL, signaling chain homooligomerization; TREM-1, triggering receptor expressed on myeloid cells-1.

FIG. 3 presents a schematic representation of one embodiment of a TREM-1-related trifunctional peptide (TREM-1/TRIOPEP) of the present invention comprising amino acid domains A and B. Depending on lipid mixture compositions added to the peptides, sub 50 nm-sized SLP particles of discoidal (TREM-1/TRIOPEP-dSLP) or spherical (TREM-1/TRIOPEP-sSLP) morphology are self-assembled upon binding of the trifunctional peptide to lipids. Abbreviations: apo, apolipoprotein; SCHOOL, signaling chain homooligomerization; TREM-1, triggering receptor expressed on myeloid cells-1.

FIG. 4A illustrates a hypothesized molecular mechanism of action of one embodiment of a trifunctional peptide (TRIOPEP) of the present invention comprising amino acid domains A and B where domain A represents a 9 amino acids-long TREM-1 inhibitory therapeutic peptide sequence and functions to treat and/or prevent a TREM-1-related disease or condition (shown for atherosclerosis), whereas domain B represents a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of atherosclerosis. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in intraplaque macrophages is accompanied by accumulation of TRIOPEP in these cells. In contrast, native HDL particles that contain only unmodified apolipoprotein molecules are not recognized by intraplaque macrophages and return to the circulation.

FIG. 4B illustrates a hypothesized molecular mechanism of action of one embodiment of a trifunctional peptide (TRIOPEP) of the present invention comprising amino acid domains A and B where domain A is a 9 amino acids-long TREM-1 inhibitory therapeutic peptide sequence and functions to treat and/or prevent a TREM-1-related disease or condition (shown for cancer), whereas domain B is a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of cancer. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in tumor-associated macrophages is accompanied by accumulation of TRIOPEP in these cells. In contrast, native HDL particles that contain only unmodified apolipoprotein molecules are not recognized by tumor-associated macrophages and return to the circulation.

FIG. 4C shows a symbol key used in FIGS. 4A-B.

FIG. 5 illustrates one embodiment of a specific disruption of intramembrane interactions between TREM-1 and DAP-12 by the trifunctional peptide of the present invention comprising two amino acid domains A and B where domain A is a 9 amino acids-long TREM-1 inhibitory therapeutic peptide sequence, whereas domain B is a 22 amino acids-long apolipoprotein A-I helix 4 or 6 peptide sequence with a sulfoxidized methionine residue. While not being bound to any particular theory, it is believed that this disruption results in “pre-dissociation” of a receptor complex and upon ligand stimulation, leads to inhibition of TREM-1 and silencing the TREM-1 signaling pathway.

FIG. 6A-C shows images depicting colocalization of the sulfoxidized methionine residue-containing TREM-1-related trifunctional peptide (TREM-1/TRIOPEP) GE31 with TREM-1 in the cell membrane (FIG. 6A), TREM-1 immunohistochemistry staining (FIG. 6B) and a merged image (FIG. 6C).

FIG. 7A-B presents the exemplary data showing the endocytosis of synthetic lipopeptide particles (SLP) of discoidal (dSLP) and spherical (sSLP) morphology that contain an equimolar mixture of the TREM-1-related trifunctional peptides (TREM-1/TRIOPEP) GA 31 and GE 31 (TREM-1/TRIOPEP-dSLP and TREM-1/TRIOPEP-sSLP, respectively). (FIG. 7A) The post 4 h incubation in vitro macrophage uptake of TREM-1/TRIOPEP-dSLP and TREM-1/TRIOPEP-sSLP with the unmodified (patterned bars) or sulfoxidized TREM-1/TRIOPEP methionine residues (black bars). ***, P=0.0001 to 0.001 (sulfoxidized vs. unmodified methionine residues). (FIG. 7B) the in vitro macrophage uptake of TREM-1/TRIOPEP-dSLP and TREM-1/TRIOPEP-sSLP with the sulfoxidized TREM-1/TRIOPEP methionine residues post 4 (white bars), 12 (patterned bars), and 24 h (black bars) incubation. ***, P=0.0001 to 0.001 as compared with 4 h incubation time.

FIG. 8 presents the exemplary data showing suppression of tumor necrosis factor-alpha (TNF-alpha), interleukin (IL)-6 and IL-1beta production by lipopolysaccharide (LPS)-stimulated macrophages incubated for 24 hour (hr) at 37° C. with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. ***, P=0.0001 to 0.001 as compared with medium-treated LPS-challenged macrophages.

FIG. 9A-C presents the exemplary data showing that scavenger receptors SR-A and SR-B1 mediate the macrophage endocytosis of GF9-sSLP (GF9-HDL) and GA/E31-HDL (TREM-1/TRIOPEP-sSLP). (FIG. 9A) Schematic representation of TREM-1 signaling and the SCHOOL mechanism of TREM-1 blockade. (FIG. 9A1, left panel) Activation of the TREM-1/DAP12 receptor complex expressed on macrophages leads to phosphorylation of the DAP12 cytoplasmic signaling domain and subsequent downstream inflammatory cytokine response (left panel). SR-mediated endocytosis of sSLP-bound GF9, GA31 and GE31 peptide inhibitors by macrophages results in the release of GF9 or GA31 and GE31 into the cytoplasm, which self-penetrate into the cell membrane and block intramembrane interactions between TREM-1 and DAP12, thereby preventing DAP12 phosphorylation and downstream signaling cascade (FIG. 9A1, right panel). (FIG. 9A2, left panel) Activation of the TREM-1/DAP12 receptor complex expressed on Kupffer cells leads to phosphorylation of the DAP12 cytoplasmic signaling domain, subsequent SYK recruitment, and the downstream inflammatory cytokine response. (FIG. 9A2, right panel) SR-mediated endocytosis of HDL-bound GF9 peptide inhibitors by Kupffer cells results in the release of GF9 (GA31 or GE31) into the cytoplasm; GF9 self-penetrates the cell membrane and blocks intramembrane interactions between TREM-1 and DAP12, thereby preventing DAP12 phosphorylation and the downstream signaling cascade.

FIG. 9B-9C Macrophage endocytosis of GF9-sSLP (GF9-HDL) and GA/E31-HDL (TREM-1/TRIOPEP-sSLP) in vitro is SR-mediated in a time-dependent manner and is largely driven by SR-A (FIG. 9B, FIG. 9C). As described in the Materials and Methods, J774 macrophages were cultured at 37° C. overnight with medium. Prior to uptake of GF9-HDL and GA/E31-HDL, cells were treated for 1 hour at 37° C. with 40 μM cytochalasin D and either (FIG. 9B) 400 μg/mL fucoidan or (FIG. 9C) 10 μM BLT-1, as indicated. Cells were then incubated for either 4 hours or 22 hours with medium containing 2 μM rhodamine B (rho B)-labeled GF9-sSLP (gray bars) or TREM-1/TRIOPEP-sSLP (black bars), respectively. Cells were lysed, and rho B fluorescence intensities of lysates were measured and normalized to the protein content. Results are expressed as mean SEM (n=3); *P≤0.05; **P≤0.01; ****P≤0.0001 versus uptake of GF9-HDL and GA/E31-HDL in the absence of inhibitor. Abbreviations: D, DAP12; DAP12, DNAX activation protein of 12 kDa; K, Kupffer cell; RFU, relative fluorescence units; SCHOOL, signaling chain homo-oligomerization.

FIG. 10 presents the exemplary data showing suppression of tumor necrosis factor-alpha (TNF-alpha), interleukin (IL)-6 and IL-1beta production in mice at 90 min post lipopolysaccharide (LPS) challenge treated 1 h before LPS challenge with phosphate-buffer saline (PBS), dexamethasone (DEX), control peptide and with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. Control peptide represents an equimolar mixture of two peptides, each of them comprising two amino acid domains A and B where domain A represents a non-functional 9 amino acids-long sequence of the TREM-1 inhibitory therapeutic peptide sequence wherein, Lys₅ is substituted with Ala₅, whereas domain B is a sulfoxidized methionine residue-containing 22 amino acids-long apolipoprotein A-I helix 4 or 6 peptide sequence, respectively. *, P=0.01 to 0.05 as compared with animals treated with 5 mg/kg TRIOPEP in free form; ***, P=0.0001 to 0.001 as compared with PBS-treated animals.

FIG. 11A-B presents the exemplary data showing inhibition of tumor growth in the human non-small cell lung cancer H292 (FIG. 11A) and A549 (FIG. 111B) xenograft mice treated with an equimolar mixture of the sulfoxidized methionine residue-containing TREM-1-related trifunctional peptides (TREM-1/TRIOPEP) GA 31 and GE 31 in free form. PTX, paclitaxel. ****, P<0.0001 as compared with vehicle-treated animals.

FIG. 12A-B presents the exemplary data showing inhibition of tumor growth in the human non-small cell lung cancer H292 (FIG. 12A) and A549 (FIG. 12B) xenograft mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.

FIG. 13 presents the exemplary data showing average tumor weights in the A549 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.

FIG. 14A-C presents the exemplary data showing inhibition of tumor growth (FIG. 14A) and TREM-1 blockade-mediated suppression of intratumoral macrophage infiltration (FIG. 14B, FIG. 14C) in the human pancreatic cancer BxPC-3 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. (B) F4/80 staining. Results are expressed as the mean±SEM (n=4 mice per group). *, p<0.05; **, p<0.01, ****, p<0.0001 (versus vehicle). (FIG. 14C) Representative F4/80 images from BxPC-3-bearing mice treated using different free and sSLP-bound SCHOOL TREM-1 inhibitory GF9 sequences including TREM-1/TRIOPEP-sSLP. Scale bar=200 μm.

FIG. 15A-B presents the exemplary data showing improved survival of lipopolysaccharide (LPS)-challenged mice treated with an equimolar mixture of the sulfoxidized methionine residue-containing TREM-1-related trifunctional peptides (TREM-1/TRIOPEP) GA 31 and GE 31 in free form (FIG. 15A) or incorporated into synthetic lipopeptide particles (SLP) particles of discoidal (TREM-1/TRIOPEP-dSLP) and spherical (TREM-1/TRIOPEP-sSLP) morphology. FIG. 15B. **, P=0.001 to 0.01 as compared with vehicle-treated animals.

FIG. 16 presents exemplary data showing average weights of healthy C57BL/6 mice treated with increasing concentrations of an equimolar mixture of the sulfoxidized methionine residue-containing TREM-1-related trifunctional peptides (TREM-1/TRIOPEP) GA 31 and GE 31 in free form.

FIG. 17A-B presents the exemplary data showing average clinical arthritis score (FIG. 17A) and mean body weight (BW) changes (FIG. 17B) calculated as a percentage of the difference between beginning (day 24) and final (day 38) BWs of the collagen-induced arthritis (CIA) mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. DEX, dexamethasone. *, p<0.05, **, p<0.01; ***, p<0.001 as compared with vehicle-treated or naive animals.

FIG. 18A-D presents the exemplary data showing reduction of pathological retinal neovascularization area (FIG. 18A), avascular area (FIG. 18B) and retinal TREM-1 (FIG. 18C) and M-CSF/CSF-1 (FIG. 18D) expression in the retina of the mice with oxygen-induced retinopathy (OIR) treated with an equimolar mixture of the sulfoxidized methionine residue-containing TREM-1-related trifunctional peptides (TREM-1/TRIOPEP) GA 31 and GE 31 incorporated into synthetic lipopeptide particles (TREM-1/TRIOPEP-SLP) particles of spherical morphology (TREM-1/TRIOPEP-sSLP). ***, p<0.001 as compared with vehicle-treated animals.

FIG. 19 presents exemplary data showing penetration of the blood-brain barrier (BBB) and blood-retinal barrier (BRB) by systemically (intraperitoneally) administered rhodamine B-labeled spherical self-assembled particles (sSLP) that contain Gd-containing contrast agent (Gd-sSLP) for magnetic resonance imaging (MRI), TREM-1 inhibitory peptide GF9 (GF9-sSLP) or an equimolar mixture of the sulfoxidized methionine residue-containing TREM-1-related trifunctional peptides GA 31 and GE 31 (TREM-1/TRIOPEP-sSLP).

FIG. 20A-B presents exemplary data showing TREM-1/TRIOPEP-sSLP suppresses the expression of fibrinogenesis marker molecules, FIG. 20A Pro-Collagen 1α and FIG. 20B α-Smooth Muscle Actin, at the RNA level, as measured in whole-liver lysates of mice with (alcohol-fed) and without (pair-fed) alcoholic liver disease (ALD).

* indicates significance level compared to the non-treated pair-fed (PF) group; #indicates significance level compared to the non-treated alcohol-fed group. o indicates significance level compared to the vehicle-treated alcohol-fed group. The significant levels are as follows: *, 0.05≥P≥0.01; **, 0.01≥P≥0.001; ***, 0.001≥P≥0.0001; ****, P≤0.0001.

FIG. 21A-D presents exemplary data showing that TREM-1/TRIOPEP-sSLP suppresses the production of alanine aminotransferase (ALT) in mice with alcoholic liver disease (ALD), as measured in serum of mice with (alcohol-fed) and without (pair-fed) ALD, in addition to improving indicators of liver disease and inflammation. * indicates significance level compared to the alcohol-fed group treated with vehicle-synthetic lipopeptide particles of spherical morphology that contained an equimolar mixture of PE22 and PA22 (sSLP) but no TREM-1 inhibitory peptide GF9. #indicates significance level compared to the non-treated alcohol-fed group. Liver damage after 5 weeks of alcohol feeding and effect of TREM-1 pathway inhibition in a mouse model of ALD. sSLP, 5 mg/kg treatment of TREM-1 peptide vs. TREM-1/TRIOPEP-sSLP. Cheek blood and livers were harvested at death. (FIG. 21A) Serum ALT levels were measured using a kinetic method. Exemplary data showing TREM-1/TRIOPEP-sSLP suppresses alanine aminotransferase in serum of alcohol fed mice over TREM-1 peptide alone. (FIG. 21B-D) Liver sections were stained with (B,C) Oil Red O and (FIG. 21D) H&E staining, and the lipid content was analyzed by ImageJ (FIG. 21B). * indicates significance level compared to the nontreated PF group; * indicates significance level compared to the nontreated alcohol-fed group; 0 indicates significance level compared to the vehicle-treated alcohol-fed group. The numbers of the symbols sign the significant levels as the following: **^{o}P<0.05; ^{##/oo} P<0.01; *″^{/###}P<0.001; ****P<0.0001. ***, 0.001≥P≥0.0001; ##, 0.01≥P≥0.001.

FIG. 22 presents an exemplary schematic representation of one embodiment of a TREM-1-related trifunctional peptide (TREM-1/TRIOPEP) G-HV21 of the present invention comprising amino acid domains A and B where domain A represents a 9 amino acids-long human TREM-1 inhibitory therapeutic peptide sequence GF9 and functions to treat and/or prevent a TREM-1-related disease or condition, whereas domain B represents a 12 amino acids-long amino acid sequence GV12 that contains a sulfoxidized methionine residue and is derived from human apolipoprotein A-I amino acid sequence. While not being bound to any particular theory, it is believed that a resulting amphipathic alpha helical peptide G-HV21 upon interaction with native lipoproteins, forms naturally long half-life lipopeptide/lipoprotein particles and targets these particles to myeloid cells (e.g. macrophages). Abbreviations: TREM-1, triggering receptor expressed on myeloid cells-1; TRIOPEP, trifunctional peptide.

FIG. 23 presents an exemplary schematic representation of one embodiment of a TREM-1-related trifunctional peptide (TREM-1/TRIOPEP) G-KV21 of the present invention comprising amino acid domains A and B where domain A represents a 9 amino acids-long human TREM-1 inhibitory therapeutic peptide sequence GF9 and functions to treat and/or prevent a TREM-1-related disease or condition, whereas domain B represents a 12 amino acids-long amino acid sequence WV12 that contains a sulfoxidized methionine residue and is derived from human apolipoprotein A-I amino acid sequence. While not being bound to any particular theory, it is believed that a resulting amphipathic alpha helical peptide G-KV21 upon interaction with native lipoproteins, forms naturally long half-life lipopeptide/lipoprotein particles and targets these particles to myeloid cells (e.g. macrophages). Abbreviations: TREM-1, triggering receptor expressed on myeloid cells-1; TRIOPEP, trifunctional peptide.

FIG. 24 presents an exemplary schematic representation of one embodiment of a TREM-1-related control peptide G-TE21 of the present invention comprising amino acid domains A and B where domain A represents a 9 amino acids-long human TREM-1 inhibitory therapeutic peptide sequence GF9, whereas domain B represents a 12 amino acids-long amino acid sequence TE12 that contains a sulfoxidized methionine residue and is derived from bovine serum albumin amino acid sequence. While not being bound to any particular theory, it is believed that a resulting non-amphipathic peptide G-TE21 does not interact with native lipoproteins and therefore does not form naturally long half-life lipopeptide/lipoprotein particles. Abbreviations: TREM-1, triggering receptor expressed on myeloid cells-1.

FIG. 25 presents an exemplary schematic representation of one embodiment of a TCR-related trifunctional peptide (TCR/TRIOPEP) M-VE32 of the present invention comprising amino acid domains A and B where domain A represents a 10 amino acids-long human TCR inhibitory therapeutic peptide sequence MF10 and functions to treat and/or prevent a TCR-related disease or condition, whereas domain B represents a 22 amino acids-long amino acid sequence PE22 that is derived from human apolipoprotein A-I amino acid sequence. While not being bound to any particular theory, it is believed that a resulting amphipathic alpha helical peptide M-VE32 upon interaction with native lipoproteins, forms naturally long half-life lipopeptide/lipoprotein particles. Abbreviations: TCR, T cell receptor; TRIOPEP, trifunctional peptide.

FIG. 26 presents a schematic representation of one embodiment of a TCR-related control peptide M-TK32 of the present invention comprising amino acid domains A and B where domain A represents a 10 amino acids-long human TCR inhibitory therapeutic peptide sequence MF10, whereas domain B represents a random 22 amino acids-long amino acid sequence LK22. While not being bound to any particular theory, it is believed that a resulting non-amphipathic peptide M-TK32 does not interact with native lipoproteins and therefore does not form naturally long half-life lipopeptide/lipoprotein particles. Abbreviations: TCR, T cell receptor.

FIG. 27 presents an exemplary schematic representation and the exemplary data showing that ultracentrifugation of whole mouse serum with added rho B-labeled TREM-1-related trifunctional peptides (TREM-1/TRIOPEP) G-HV21 and G-KV21 results in floatation of these peptides with mouse lipoproteins. In contrast, when added to whole mouse serum, rho B-labeled TREM inhibitory peptide GF9 or rho B-labeled TREM-1-related control peptide G-TE21 sedimentate with serum proteins upon ultracentrifugation. When added to delipoproteinized mouse serum that does not contain lipoproteins, rho B-labeled TREM-1/TRIOPEP G-HV21 and G-KV21 sedimentate with serum proteins upon ultracentrifugation. While not being bound to any particular theory, it is believed that TREM-1/TRIOPEP G-HV21 and G-KV21 interact with native lipoproteins of a whole mouse serum and/or their lipid components and form lipopeptide/lipoprotein particles that mimic serum lipoproteins and float under the same ultracentrifugation conditions. Abbreviations: TREM-1, triggering receptor expressed on myeloid cells-1; rho B, rhodamine B.

FIG. 28 presents exemplary data showing the endocytosis of rho B-labeled GF9, G-TE21, G-HV-21 and G-KV21 by macrophages in the absence (white bars) or presence (black bars) of HDL. In contrast to GF9 and TREM-1-related control peptide G-TE21, the in vitro macrophage uptake of TREM-1/TRIOPEP G-HV21 and G-KV21 significantly increases in the presence of HDL. ***, p<0.001 (presence vs. absence of HDL). Abbreviations: HDL, high density lipoproteins; rho B, rhodamine B; n.s., not significant.

FIG. 29A-C shows exemplary images depicting colocalization of the sulfoxidized methionine residue-containing TREM-1/TRIOPEP G-KV21 (pre-incubated with HDL) with TREM-1 in the J774 cell membrane FIG. 29A. FIG. 29B TREM-1 immunostaining. FIG. 29C merged image. Abbreviations: TREM-1, triggering receptor expressed on myeloid cells-1; HDL, high density lipoproteins.

FIG. 30A illustrates a hypothesized molecular mechanism of action of TREM-1-related trifunctional peptides (TREM-1/TRIOPEP) of the present invention (shown for atherosclerosis). While not being bound to any particular theory, it is believed that upon interaction with native lipoproteins including HDL, the modified methionine residue in the TREM-1/TRIOPEP domain B mediates the recognition of the formed lipopeptide/lipoprotein particles by macrophage scavenger receptors and results in an irreversible binding to and consequent uptake by macrophages of such particles. It is further believed that accumulation of these particles in intraplaque macrophages is accompanied by accumulation of TREM-1/TRIOPEP released within these cells. In contrast, native HDL particles are not recognized by intraplaque macrophages and return to the circulation.

FIG. 30B Abbreviations: TREM-1, triggering receptor expressed on myeloid cells-1; HDL, high-density lipoproteins.

FIG. 31A illustrates a hypothesized molecular mechanism of action of TREM-1-related trifunctional peptides (TREM-1/TRIOPEP) of the present invention (shown for cancer). While not being bound to any particular theory, it is believed that upon interaction with native lipoproteins including HDL, the modified methionine residue in the TREM-1/TRIOPEP domain B mediates the recognition of the formed lipopeptide/lipoprotein particles by macrophage scavenger receptors and results in an irreversible binding to and consequent uptake by macrophages of such particles. It is further believed that accumulation of these particles in tumor-associated macrophages is accompanied by accumulation of TREM-1/TRIOPEP released within these cells. In contrast, native HDL particles are not recognized by intraplaque macrophages and return to the circulation.

FIG. 31B Abbreviations: TREM-1, triggering receptor expressed on myeloid cells-1; HDL, high-density lipoproteins.

FIG. 32 illustrates one embodiment of a specific disruption of intramembrane interactions between TREM-1 and DAP-12 by the TREM-1-related trifunctional peptide (TREM-1/TRIOPEP) of the present invention delivered to and released within TREM-1-expressing cells by the lipopeptide/lipoprotein particles formed upon interaction of TREM-1/TRIOPEP with native lipoproteins. While not being bound to any particular theory, it is believed that this disruption results in “pre-dissociation” of a receptor complex and upon ligand stimulation, leads to inhibition of TREM-1 and silencing the TREM-1 signaling pathway. Abbreviations: TREM-1, triggering receptor expressed on myeloid cells-1; DAP-12, DNAX-activation protein 12; M-CSF/CSF-1, macrophage colony stimulating factor-1; MCP-1/CCL2, monocyte chemoattractant protein-1; IL, interleukin; TNF, tumor necrosis factor.

FIG. 33 presents exemplary data showing cytokine production by LPS-stimulated macrophages incubated for 24 h at 37° C. with GF9, G-TE21, G-HV21 and G-KV21 in the presence of HDL. In contrast to GF9 and TREM-1-related control peptide G-TE21, TREM-1/TRIOPEP G-HV21 and G-KV21 significantly inhibit the cytokine release in the presence of HDL. In the absence of HDL, G-HV21 does not affect the cytokine production. ***, p<0.001 (vs. medium+HDL). Abbreviations: TREM-1, triggering receptor expressed on myeloid cells-1; LPS, lipopolysaccharide; IL, interleukin; TNF, tumor necrosis factor; HDL, high density lipoproteins.

FIG. 34A-C presents exemplary LPS-challenged J774 macrophages: Cytokine release data showing that scavenger receptors SR-A and SR-B1 mediate the macrophage endocytosis of TREM-1/TRIOPEP G-HV21 and G-KV21 in the presence of HDL. (FIG. 34A) Schematic representation of TREM-1 signaling and the SCHOOL mechanism of TREM-1 blockade. Activation of the TREM-1/DAP12 receptor complex expressed on macrophages leads to phosphorylation of the DAP12 cytoplasmic signaling domain and subsequent downstream inflammatory cytokine response (left panel). SR-mediated macrophage endocytosis of the lipopeptide/lipoprotein particles formed upon interaction of TREM-1/TRIOPEP with native lipoproteins (shown for HDL) results in the release of TREM-1/TRIOPEP into the cytoplasm. Then, the released TREM-1/TRIOPEP self-penetrate into the cell membrane and block intramembrane interactions between TREM-1 and DAP12, thereby preventing DAP12 phosphorylation and downstream signaling cascade (FIG. 34A, right panel). Macrophage endocytosis of G-HV21 and G-KV21 in the presence of HDL in vitro is SR-mediated in a time-dependent manner and is largely driven by SR-A (B, C). J774 macrophages were cultured at 37° C. overnight with medium. Before adding G-HV21 and G-KV21, cells were treated for 1 h at 37° C. with 40 μM cytochalasin D, 400 μg/mL fucoidan (FIG. 34B) or 10 μM BLT-1 (FIG. 34C) as indicated. Cells were then incubated for either 4 h or 22 h with medium containing HDL and 2 M rho B-labeled G-KV21 (gray bars) or G-HV21 (black bars), respectively. Cells were lysed and rho B fluorescence intensities of lysates were measured and normalized to the protein content. Results are expressed as the mean±SEM (n=3). *, p<0.05; **, p<0.01; ****, p<0.0001 versus uptake of G-HV21 and G-KV21 in the absence of inhibitor. Abbreviations: TREM-1, triggering receptor expressed on myeloid cells-1; DAP-12, DNAX-activation protein 12; M-CSF/CSF-1, macrophage colony stimulating factor; MCP-1/CCL2, monocyte chemoattractant protein-1; IL, interleukin; TNF, tumor necrosis factor; HDL, high density lipoproteins; BLT-1, blocker of lipid transport-1; rho B, rhodamine B; SR, scavenger receptor.

FIG. 35 presents exemplary data showing serum cytokine production at 90 min post LPS challenge in mice treated at 1 h before LPS challenge with PBS, DEX, GF9, TREM-1-related control peptide G-TE21 or with TREM-1-related trifunctional peptides G-HV21 and G-KV21. In contrast to GF9 and G-TE21, G-HV21 and G-KV21 significantly inhibit the LPS-induced cytokine release. ***, p<0.001 as compared with PBS-treated animals.

Abbreviations: TREM-1, triggering receptor expressed on myeloid cells-1; LPS, lipopolysaccharide; IL, interleukin; TNF, tumor necrosis factor; DEX, dexamethasone; PBS, phosphate-buffer saline.

FIG. 36A-B presents the exemplary data showing survival of LPS-challenged mice treated with PBS (vehicle), TREM-1-related control peptide G-TE21, TREM-1-related trifunctional peptides G-HV21 and G-KV21 (FIG. 36A) or with TREM-1 inhibitory peptide GF9 at different doses (FIG. 36B). In contrast to G-TE21, G-HV21 and G-KV21 significantly improve survival of septic mice (FIG. 36A). When administered at a dose of 5 mg/kg, GF9 does not affect survival of septic mice, while at 25 mg/g, GF9 improves survival. In contrast, high dose of GF9, 150 mg/kg, contributes to earlier death as compared with control animals treated with vehicle only (FIG. 36B). **, p<0.01 as compared with vehicle-treated animals. Abbreviations: TREM-1, triggering receptor expressed on myeloid cells-1; LPS, lipopolysaccharide; PBS, phosphate-buffer saline.

FIG. 37A-B presents the exemplary data showing tumor growth in the human non-small cell lung cancer H292 mouse xenograft (FIG. 37A) and A549 mouse xenograft (FIG. 37B) xenograft mice treated with PBS (vehicle), PTX, TREM-1-related control peptide G-TE21 or with TREM-1-related trifunctional peptides G-HV21 and G-KV21. In contrast to G-TE21, G-HV21 and G-KV21 significantly inhibit the tumor growth. ****, p<0.0001 as compared with vehicle-treated animals. Abbreviations: PTX, paclitaxel; PBS, phosphate-buffer saline.

FIG. 38 presents exemplary A549 mouse xenograft data showing average tumor weights in the A549 xenograft mice treated with PBS (vehicle), PTX, TREM-1-related control peptide G-TE21 or with TREM-1-related trifunctional peptides G-HV21 and G-KV21. In contrast to G-TE21, G-HV21 and G-KV21 significantly decrease the tumor weight. ** p<0.01 as compared with vehicle-treated animals. Abbreviations: TREM-1, triggering receptor expressed on myeloid cells-1; PTX, paclitaxel; PBS, phosphate-buffer saline; n.s., not significant.

FIG. 39A-B presents exemplary data showing tumor growth (A) and, infiltration of macrophages into the tumor as evaluated by F4/80 staining (B) in the human pancreatic cancer BxPC-3 xenograft mice treated with PBS (vehicle), TREM-1-related control peptide G-TE21 or with TREM-1-related trifunctional peptides G-HV21 and G-KV21. In contrast to G-TE21, G-HV21 and G-KV21 in a BxPC-3 mouse xenograft significantly inhibits the tumor growth (FIG. 389) and reduce macrophage infiltration into the tumor (FIG. 39B). **, p<0.01, ****, p<0.0001 (versus vehicle). Abbreviations: TREM-1, triggering receptor expressed on myeloid cells-1; PBS, phosphate-buffer saline; n.s., not significant.

FIG. 40A-B presents exemplary data showing PANC-1 mouse xenograft tumor growth (FIG. 40A) and survival (FIG. 40B) in the human pancreatic cancer PANC-1 xenograft mice treated with PBS (vehicle) and TREM-1-related trifunctional peptide G-KV21 with or without chemotherapy treatment (GEM+ABX). G-KV21 sensitizes the tumor to chemotherapy (FIG. 40A) and significantly improves survival (FIG. 40B). The median survival times (FIG. 40B) are indicated in parentheses. Abbreviations: TREM-1, triggering receptor expressed on myeloid cells-1; PBS, phosphate-buffer saline; GEM, gemcitabine; ABX, Abraxane (nanoparticle albumin-bound paclitaxel).

FIG. 41 presents the exemplary data showing average weights of Healthy C57BL/6 mice treated with TREM-1-related control peptide G-TE21 or with TREM-1-related trifunctional peptides G-HV21 and G-KV21. No toxicity was observed for all three peptides. Abbreviations: TREM-1, triggering receptor expressed on myeloid cells-1.

FIG. 42A-B presents exemplary data showing average clinical arthritis score (Collagen-induced arthritis: Score FIG. 42A) and Collagen-induced arthritis: Body weight change mean BW changes (FIG. 42B) calculated as a percentage of the difference between beginning (day 24) and final (day 38) BWs of the CIA mice treated with PBS (vehicle), DEX, TREM-1-related control peptide G-TE21, TCR-related control peptide M-TK32, TCR-related trifunctional peptide M-VE32 or with TREM-1-related trifunctional peptides G-HV21 and G-KV21. In contrast to the relevant control peptides, G-HV21, G-KV21 and M-VE32 all ameliorate the disease (FIG. 42A) and are well-tolerated by arthritic mice (FIG. 42B). *, p<0.05, **, p<0.01; ***, p<0.001 as compared with vehicle-treated animals. Abbreviations: TREM-1, triggering receptor expressed on myeloid cells-1; CIA, collagen-induced arthritis; PBS, phosphate-buffer saline; DEX, dexamethasone; TCR, T cell receptor; BW, body weight.

FIG. 43A-D Oxygen-induced retinopathy presents exemplary data showing pathological RNV (FIG. 43A) and avascular (FIG. 43B) areas as well as expression of TREM-1 (FIG. 43C) and M-CSF (FIG. 43D) in the retina of the mice with OIR treated with PBS (vehicle), TREM-1-related control peptide G-TE21 or TREM-1-related trifunctional peptide G-KV21. In contrast to G-TE21, G-KV21 significantly suppresses pathological RNV and inhibits tissue expression of TREM-1 and M-CSF. *, p<0.05, **, p<0.01; ***, p<0.001 as compared with vehicle-treated animals. Abbreviations: TREM-1, triggering receptor expressed on myeloid cells-1; OIR, oxygen-induced retinopathy; PBS, phosphate-buffer saline; M-CSF, macrophage colony stimulating factor; RNV, retinal neovascularization.

FIG. 44 presents exemplary data showing penetration of the BBB and BRB by systemically (mice—intraperitoneally; rats and rabbits—intravenously) administered rhodamine B-labeled TREM-1-related trifunctional peptide G-KV21. Abbreviations: TREM-1, triggering receptor expressed on myeloid cells-1; BBB, blood-brain barrier; BRB, blood-retinal barrier.

FIG. 45A-E TREM-1 pathway inhibition. TREM-1 pathway inhibition suppresses the expression of (FIG. 45A) TREM-1 and inflammatory cytokines (FIG. 45B) MCP-1, (FIG. 45C) TNF-α, (FIG. 45D) IL-1, and (FIG. 45E) MIP-1α but not (FIG. 45F) RANTES at the mRNA level as measured in whole-liver lysates by real-time quantitative PCR. * indicates significance level compared to nontreated PF group; #indicates significance level compared to nontreated alcohol-fed group; o indicates significance level compared to vehicle-treated alcohol-fed group. Significance levels are as follows: */#/o P≤0.05; **/##/oo P≤0.01; ***/ooo P≤0.001; ****P≤0.0001. Abbreviation: CCL, chemokine (C—C motif) ligand.

FIG. 46AE-G TREM-1 blockade and inflammatory cytokine levels. TREM-1 blockade reduces inflammatory cytokine levels in (FIG. 46A) serum and (FIG. 46B-D) whole-liver lysates as measured with specific ELISA kits. (FIG. 46E-G) Total liver protein was analyzed for total SYK and activated p-SYK Y525/526 expression by western blotting using j-actin as a loading control. Statistical analysis was performed by evaluating two blots (n=4/group). * indicates significance level compared to the nontreated PF group; #indicates significance level compared to the nontreated alcohol-fed group; o indicates significance level compared to the vehicle-treated alcohol-fed group. Significance levels are as follows: */#/o P≤0.05; **/##P≤0.01; ***P≤0.001; ****/####P≤0.0001.

FIG. 47A-H Effects of TREM-1 inhibition. (FIG. 47A, FIG. 47B) TREM-1 inhibition suppresses the mRNA expression of macrophage cell markers in the liver as measured by real-time quantitative PCR. (FIG. 47C, FIG. 47D) Both TREM-1 inhibitors attenuated F4/80 as shown by IHC. (FIG. 47E, FIG. 47F) TREM-1 inhibition suppresses the mRNA expression of neutrophil cell markers in the liver as measured by real-time quantitative PCR. (FIG. 47G, H) Both TREM-1 inhibitors attenuated MPO-positive cell infiltration as shown by IHC. * indicates significance level compared to the nontreated PF group; #indicates significance level compared to the nontreated alcohol-fed group; o indicates significance level compared to the vehicle-treated alcohol-fed group. Significance levels are as follows: */#/o P≤0.05; **/##P≤0.01; ###P≤0.001; ****/####P≤0.0001.

FIG. 48A-F Measurement of mRNA expression. mRNA expression of genes involved in (FIG. 48A, FIG. 48B) lipid synthesis (SERBF1, ACC1), (FIG. 48C) the lipid accumulation marker (ADRP), and (FIG. 48D-F) lipid oxidation (PPARα, CPT1α, MCAD) were measured in whole liver.

* indicates significance level compared to the nontreated PF group; #indicates significance level compared to the nontreated alcohol-fed group; o indicates significance level compared to the vehicle-treated alcohol-fed group. Significance levels are as follows: */#/o P≤0.05; **/##/oo P≤0.01; ###P≤0.001; ****P≤0.0001.

FIG. 49A presents a schematic representation of one embodiment of the proposed role of inhibition of TREM-1 expressed on tumor-associated macrophages (TAMs) in pancreatic cancer. Pancreatic ductal adenocarcinoma cells, cancer-associated fibroblasts (CAFs) and TAMs play a role in generating a tumor favorable microenvironment, in part by producing such cytokines and growth factors as interleukin (IL)-1α, IL-6 and macrophage colony-stimulating factor (M-CSF).

FIG. 49B presents a schematic representation of one embodiment of suppressing tumor favorable microenvironment by inhibition of TREM-1 expressed on tumor-associated macrophages (TAMs) and reduction of cytokines and growth factors including but not limited to interleukin (IL)-6, IL-1, monocyte chemoattractant protein-1 (MCP-1; also referred to in the art as CCL2) and macrophage colony-stimulating factor 1 (CSF-1; also referred to in the art as M-CSF). These prognostic factors are involved in tumorigenesis, cancer progression, metastasis, and even in the response to cancer treatment. The figure further presents a schematic representation of one embodiment of modulating the TREM-1/DAP-12 signaling pathway by type I TREM-1 inhibitors that bind either TREM-1 (type Ia inhibitors; e.g., anti-TREM-1 blocking antibodies, etc.) or its ligand (type Ib inhibitors; e.g., inhibitory peptides LP17 and LR12 that act as a decoy TREM-1 receptor), thereby blocking binding between TREM-1 and its yet uncertain ligand(s).

FIG. 50 presents a schematic representation of one embodiment of TREM-1 modulatory peptide variants and compositions of the present invention that are rationally designed using the Signaling Chain HOmoOLigomerization (SCHOOL approach) to inhibit TREM-1 in a ligand-independent manner by blocking intramembrane interactions between TREM-1 and its signaling partner DAP-12 (type II inhibitors). These SCHOOL peptides can be employed in either free form or incorporated into macrophage-targeted (macrophage-specific) synthetic lipopeptide particles (SLP), which allows them to reach their site of action from either outside (Route 1) or inside the cell (Route 2).

FIG. 51A-F shows images of one embodiment depicting colocalization of the TREM-1 modulatory peptide GF9 (GFLSKSLVF) with trifunctional TREM-1 in the cell membrane. FIG. 51A shows exemplary peptide GF9. FIGS. 51B and 51E shows exemplary TREM-1. FIGS. 51C and F shows exemplary merged Images. FIG. 51A shows exemplary inhibitory peptide GE31 ((GFLSKSLVFPYLDDFQKKWQEEM(O)ELYRQKVE) where M(O) is a methionine sulfoxide residue with TREM-1 in the cell membrane.

FIG. 51B shows images of one embodiment depicting colocalization of the TREM-1 modulatory peptide GF9 (GFLSKSLVF) and trifunctional TREM-1

FIG. 52 presents the exemplary data of one embodiment showing that treatment with free TREM-1 modulatory peptide GF9 (GFLSKSLVF) or GF9 incorporated into the a carrier, e.g. synthetic lipopeptide particle (SLP) of discoidal (dSLP) or spherical (sSLP) morphology suppresses tumor growth in experimental pancreatic cancer. As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GF9 (dark gray squares), GF9-loaded discoidal SLP (GF9-dSLP, light gray circles) or GF9-loaded spherical SLP (GF9-sSLP, white circles) at indicated doses. Treatment persisted for 31, 29 and 29 days for mice containing AsPC-1, BxPC-3 and Capan-1 tumor xenografts, respectively. Mean tumor volumes are calculated and plotted. All results are expressed as the mean±SEM (n=6 mice per group). On the final day of treatment, tumor volumes were compared between the drug-treated and control groups. **, p<0.01; ***, p<0.001; ****, p<0.0001 (versus vehicle).

FIG. 53 presents the exemplary data of one embodiment showing that treatment with free TREM-1 modulatory peptide GF9 (GFLSKSLVF) or GF9 incorporated into the a carrier, e.g. synthetic lipopeptide particle (SLP) of discoidal (dSLP) or spherical (sSLP) morphology (GF9-dSLP and GF9-sSLP, respectively) suppresses tumor growth in experimental pancreatic cancer without affecting body weight (well-tolerable in long term-treated mice). As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GF9 (dark gray squares), GF9-dSLP (light gray circles) or GF9-sSLP (white circles) at indicated doses. Treatment persisted for 31, 29 and 29 days for mice containing AsPC-1, BxPC-3 and Capan-1 tumor xenografts, respectively. Body weighs are plotted. All results are expressed as the mean±SEM (n=6 mice per group).

FIG. 54 presents the exemplary data of one embodiment showing that treatment with synthetic lipopeptide particle (SLP) of discoidal (dSLP) or spherical (sSLP) morphology loaded with an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-dSLP and GA/E31-sSLP, respectively) suppresses tumor growth in experimental pancreatic cancer. As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GA/E31-dSLP (light gray triangles) or GA/E31-sSLP (white triangles) at indicated doses. Treatment persisted for 31, 29 and 29 days for mice containing AsPC-1, BxPC-3 and Capan-1 tumor xenografts, respectively. Mean tumor volumes are calculated and plotted. All results are expressed as the mean±SEM (n=6 mice per group). On the final day of treatment, tumor volumes were compared between the drug-treated and control groups. **, p<0.01; ***, p<0.001; ****, p<0.0001 (versus vehicle).

FIG. 55 presents the exemplary data of one embodiment showing that treatment with synthetic lipopeptide particle (SLP) of discoidal (dSLP) or spherical (sSLP) morphology loaded with an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-dSLP and GA/E31-sSLP, respectively) suppresses tumor growth in experimental pancreatic cancer without affecting body weight (i.e. well tolerable by long term-treated mice). As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GA/E31-dSLP (light gray triangles) or GA/E31-sSLP (white triangles) at indicated doses. Treatment persisted for 31, 29 and 29 days for mice containing AsPC-1, BxPC-3 and Capan-1 tumor xenografts, respectively. Body weighs are plotted. All results are expressed as the mean±SEM (n=6 mice per group).

FIG. 56 presents the exemplary data of one embodiment showing that treatment with free TREM-1 modulatory peptide GF9 (GFLSKSLVF) or GF9 incorporated into synthetic lipopeptide particle (SLP) of discoidal (dSLP) or spherical (sSLP) morphology (GF9-dSLP and GF9-sSLP, respectively) prolongs survival in experimental pancreatic cancer. As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GF9 (dark gray circles), GF9-dSLP (light gray circles) or GF9-sSLP (white circles) at indicated doses. Treatment persisted for 31, 29 and 29 days for mice containing AsPC-1, BxPC-3 and Capan-1 tumor xenografts, respectively. Kaplan-Meier survival curves are shown for AsPC-1-, BxPC-3- or Capan-1-bearing mice (n=6 mice per group). **, p<0.01; ***, p<0.001 by log-rank test (versus vehicle).

FIG. 57 presents the exemplary data of one embodiment showing that treatment with synthetic lipopeptide particle (SLP) of discoidal (dSLP) or spherical (sSLP) morphology loaded with an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-dSLP and GA/E31-sSLP, respectively) prolongs survival in experimental pancreatic cancer. As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GA/E31-dSLP (light gray triangles) or GA/E31-sSLP (white triangles) at indicated doses. Treatment persisted for 31, 29 and 29 days for mice containing AsPC-1, BxPC-3 and Capan-1 tumor xenografts, respectively. Kaplan-Meier survival curves are shown for AsPC-1-, BxPC-3- or Capan-1-bearing mice (n=6 mice per group). **, p<0.01; ***, p<0.001 by log-rank test (versus vehicle).

FIG. 58 presents the exemplary data of one embodiment showing that the antitumor efficacy of TREM-1 blockade correlates with the intratumoral macrophage content in experimental pancreatic cancer. Antitumor efficacy is expressed as percent treatment/control (% T/C) values calculated using the following formula: % T/C=100×ΔT/ΔC where T and C are the mean tumor volumes of the drug-treated and control groups, respectively, on the final day of the treatment; ΔT is the mean tumor volume of the drug-treated group on the final day of the treatment minus mean tumor volume of the drug-treated group on initial day of dosing; and ΔC is the mean tumor volume of the control group on the final day of the treatment minus mean tumor volume of the control group on initial day of dosing. Intratumoral macrophage content was quantified by F4/80 staining using F4/80 antibodies. Data are shown for the groups of AsPC-1-, BxPC-3- and Capan-1-bearing mice treated with free and SLP-bound TREM-1 modulatory peptides GF9 (GFLSKSLVF), GA31 (GFLSKSLVFPLGEEM(O)RDRARAHVDALRTHLA where M(O) is a methionine sulfoxide residue) and GE31 (GFLSKSLVFPYLDDFQKKWQEEM(O)ELYRQKVE where M(O) is a methionine sulfoxide residue) (GA/E31-sSLP) (n=4 mice per group).

FIG. 59 presents the exemplary data of one embodiment showing that TREM-1 blockade suppresses intratumoral macrophage infiltration in experimental pancreatic cancer. Intratumoral macrophage content was quantified by F4/80 staining using F4/80 antibodies. Data are shown for the groups of BxPC-3-bearing mice treated with either vehicle (black bars), free GF9 (GFLSKSLVF, dark grey bars), GF9 incorporated into a carrier, e.gs. synthetic lipopeptide particle of spherical morphology (GF9-sSLP, light grey bars) and sSLP that contain an equimolar mixture of TREM-1 modulatory peptides GA31 (GFLSKSLVFPLGEEM(O)RDRARAHVDALRTHLA where M(O) is a methionine sulfoxide residue) and GE31 (GFLSKSLVFPYLDDFQKKWQEEM(O)ELYRQKVE where M(O) is a methionine sulfoxide residue) (GA/E31-sSLP, white bars) (n=4 mice per group).

FIG. 60 presents the exemplary data of one embodiment showing the representative F4/80 images demonstrating that TREM-1 blockade suppresses intratumoral macrophage infiltration in experimental pancreatic cancer. Intratumoral macrophage content was quantified by F4/80 staining using F4/80 antibodies. Data are shown for the groups of BxPC-3-bearing mice treated with either vehicle, free GF9 (GFLSKSLVF), GF9 incorporated into synthetic lipopeptide particle of spherical morphology (GF9-sSLP) and sSLP that contain an equimolar mixture of TREM-1 modulatory peptides GA31 (GFLSKSLVFPLGEEM(O)RDRARAHVDALRTHLA where M(O) is a methionine sulfoxide residue) and GE31 (GFLSKSLVFPYLDDFQKKWQEEM(O)ELYRQKVE where M(O) is a methionine sulfoxide residue) (GA/E31-sSLP) (n=4 mice per group).

FIG. 61 presents the exemplary data of one embodiment showing that TREM-1 blockade suppresses serum proinflammatory cytokines in xenograft mouse models of pancreatic cancer. Serum interleukin-1α (IL-1α), IL-6 and macrophage colony-stimulating factor (M-CSF/CSF-1) levels were analyzed on study days 1 and 8 in AsPC-1-, BxPC-3- and Capan-1-bearing mice treated daily 5 times per week (5qw) with either vehicle (black diamonds), GF9 (dark gray squares) or GF9-loaded spherical synthetic lipopeptide particles (GF9-sSLP, white circles) at indicated doses. Results are expressed as the mean±SEM (n=5 mice per group). *, p<0.05; **, p<0.01; ***, p<0.001; ****, p<0.0001 (versus vehicle).

FIG. 62 presents the exemplary data of one embodiment showing that treatment with free TREM-1 modulatory peptide GF9 (GFLSKSLVF) or GF9 incorporated into lipopeptide complex (GF9-LPC) suppresses serum proinflammatory cytokines colony-stimulating factor 1 (CSF1) and interleukin 6 (IL-6) but not vascular endothelial growth factor (VEGF) in the AsPC-1 xenograft mouse model of pancreatic cancer. Serum CSF1, VEGF and IL-6 levels were analyzed on study days 1 and 8 in AsPC-1-bearing mice treated daily 5 times per week (5qw) with either vehicle (black diamonds), GF9 (white circles) or GF9-LPC (black circles) at indicated doses. Results are expressed as the mean±SEM (n=5 mice per group). *, p<0.05; **, p<0.01; ***, p<0.001; ****, p<0.0001 (versus vehicle).

FIG. 63 presents the exemplary data of one embodiment showing that treatment with free TREM-1 modulatory peptide GF9 (GFLSKSLVF) or GF9 incorporated into lipopeptide complex (GF9-LPC) suppresses serum proinflammatory cytokines colony-stimulating factor 1 (CSF1) and interleukin 6 (IL-6) but not vascular endothelial growth factor (VEGF) in the BxPC-3 xenograft mouse model of pancreatic cancer. Serum CSF1, VEGF and IL-6 levels were analyzed on study days 1 and 8 in BxPC-3-bearing mice treated daily 5 times per week (5qw) with either vehicle (black diamonds), GF9 (white circles) or GF9-LPC (black circles) at indicated doses. Results are expressed as the mean±SEM (n=5 mice per group). *, p<0.05; **, p<0.01; ***, p<0.001; ****, p<0.0001 (versus vehicle).

FIG. 64 presents the exemplary data of one embodiment showing that treatment with free TREM-1 modulatory peptide GF9 (GFLSKSLVF) or GF9 incorporated into lipopeptide complex (GF9-LPC) suppresses serum proinflammatory cytokines colony-stimulating factor 1 (CSF1) and interleukin 6 (TL-6) but not vascular endothelial growth factor (VEGF) in the CAPAN-1 xenograft mouse model of pancreatic cancer. Serum CSF1, VEGF and IL-6 levels were analyzed on study days 1 and 8 in CAPAN-1-bearing mice treated daily 5 times per week (5qw) with either vehicle (black diamonds), GF9 (white circles) or GF9-LPC (black circles) at indicated doses. Results are expressed as the mean±SEM (n=5 mice per group). *, p<0.05; **, p<0.01; ***, p<0.001; ****, p<0.0001 (versus vehicle).

FIG. 65 presents the exemplary data of one embodiment showing that combining of Gemcitabine and Abraxane chemotherapy with TREM-1 modulatory peptide GF9 (GFLSKSLVF) incorporated into synthetic lipopeptide particle (SLP) of spherical (sSLP) morphology (GF9-sSLP) has a synergistic effect in experimental pancreatic cancer. As described herein, after tumors in PANC-1-bearing mice reached a volume of 150-200 mm³, mice were randomized into groups and intraperitoneally (i.p.) administered with either vehicle (black diamonds; once daily 5 times per week, 5qw), GF9-sSLP (black squares; once daily 5 times per week, 5qw), Gemcitabine and Abraxane (black circles; days 1, 4, 8, 11, 15) or GF9-sSLP (once daily 5 times per week, 5qw) in combination with Gemcitabine and Abraxane (days 1, 4, 8, 11, 15) (Black triangles). Treatment with GF9-sSLP persisted for 28 days. Mean tumor volumes are calculated and plotted. All results are expressed as the mean±SEM (n=9 mice per group). On the final day of treatment, tumor volumes were compared between the Gemcitabine+Abraxane-treated and GF9-sSLP+Gemcitabine+Abraxane-treated groups. **, p<0.01 (versus chemotherapy alone treated group).

FIG. 66 presents the exemplary data showing penetration of the blood-brain barrier (BBB) and blood-retinal barrier (BRB) by systemically (intraperitoneally) administered rhodamine B-labeled spherical synthetic lipopeptide particles (sSLP) that contain Gd-containing contrast agent (Gd-sSLP) for magnetic resonance imaging (MRI), TREM-1 modulatory peptide GF9 (GF9-sSLP) or an equimolar mixture of the sulfoxidized methionine residue-containing TREM-1 modulatory peptides, i.e. 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue GA 31 and GE 31 (GA/E31-sSLP).

FIG. 67 presents the exemplary data of one embodiment showing that TREM-1 blockade with GF9, GF9 incorporated into the carrier—spherical synthetic lipopeptide particles (GF9-sSLP) or sSLP that carried an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-sSLP) significantly reduces tissue expression of colony-stimulating factor 1 (CSF-1) and TREM-1 in the retina of mice with oxygen-induced retinopathy (OIR) at postnatal 20 day 17 (P17). Representative Western blots of retinal lysates from OIR mice are shown. The membrane was probed for TREM-1, reprobed for CSF-1 and then for β-actin. Values in the bar graphs represent the mean±SEM, n=6. *, p<0.05, **, p<0.01 vs. vehicle-treated mice.

FIG. 68 presents the exemplary data of one embodiment showing that combining gemcitabine (GEM) and abraxane (ABX) chemotherapy with TREM-1 modulatory peptide GF9 (GFLSKSLVF) incorporated into a carrier, e.g. synthetic lipopeptide particle (SLP) of spherical (sSLP) morphology (GF9-sSLP) has a synergistic therapeutic effect in experimental pancreatic cancer. As described herein, after tumors in PANC-1-bearing mice reached a volume of 150-200 mm³, mice were randomized into groups and intraperitoneally (i.p.) administered at indicated doses with either vehicle (black diamonds; once daily 5 times per week, 5qw), GF9-LPC (black circles-black squares; once daily 5 times per week, 5qw), GEM and ABX (black squares-(black circles; days 1, 4, 8, 11, 15) or GF9-LPC (once daily 5 times per week, 5qw) in combination with GEM and ABX (days 1, 4, 8, 11, 15) (half black half white hexagons-Black triangles). Treatment with GF9-LPC persisted for 28 days. Mean tumor volumes are calculated and plotted. All results are expressed as the mean±SEM (n=9 mice per group). On the day 88, tumor volumes were compared between the GEM+ABX-treated and GF9-sSLP+GEM+ABX-treated groups. *, p<0.05 (versus GEM+ABX-treated group), second set of symbols are used in the longer term studies.

FIG. 69 presents the exemplary data of one embodiment showing that TREM-1 blockade treatment with TREM-1 modulatory peptide GF9 (GFLSKSLVF) incorporated into a, e.g. synthetic lipopeptide particle (SLP) of spherical (sSLP) morphology (GF9-sSLP) alone, lipopeptide complex (GF9-LPC) alone or in combination with gemcitabine (GEM) and abraxane (ABX) chemotherapy is well tolerable in mice with human PANC-1 pancreatic cancer xenografts. As described herein, after tumors in PANC-1-bearing mice reached a volume of 150-200 mm³, mice were randomized into groups and intraperitoneally (i.p.) administered at indicated doses with either vehicle (black diamonds; once daily 5 times per week, 5qw), GF9-LPC (black circles; once daily 5 times per week, 5qw), GEM and ABX (black squares; days 1, 4, 8, 11, 15) or GF9-LPC (once daily 5 times per week, 5qw) in combination with GEM and ABX (days 1, 4, 8, 11, 15) (half black half white hexagons). Treatment with GF9-LPC (GF9-sSLP) persisted for 28 days. Body weighs are plotted. All results are expressed as the mean±SEM (n=6 mice per group).

FIG. 70 presents the exemplary data of one embodiment showing that treatment with TREM-1 modulatory peptide GF9 incorporated into a carrie, e.g. synthetic lipopeptide complex (GF9-LPC) and particle (SLP) of spherical (sSLP) morphology (GF9-sSLP), synergistically prolongs survival rate in experimental pancreatic cancer (e.g. PANC-1) when combined with gemcitabine (GEM) and abraxane (ABX) chemotherapy. As described herein, after tumors in PANC-1-bearing mice reached a volume of 150-200 mm³, mice were randomized into groups and intraperitoneally (i.p.) administered at indicated doses with either vehicle (once daily 5 times per week, 5qw), GF9-LPC (once daily 5 times per week, 5qw), GEM and ABX (days 1, 4, 8, 11, 15) or GF9-LPC (once daily 5 times per week, 5qw) in combination with GEM and ABX (days 1, 4, 8, 11, 15). Treatment with GF9-LPC persisted for 28 days. Kaplan-Meier survival curves are shown for PANC-1-bearing mice (n=6 mice per group). *, p<0.05 by log-rank test (versus GEM+ABX).

FIG. 71 presents the exemplary data of one embodiment showing that treatment with free TREM-1 modulatory peptide GF9 (GFLSKSLVF) is well tolerable in mice up to at least 300 mg/kg. As described herein, healthy C57BL/6 mice were intraperitoneally (i.p.) administered daily for 7 consecutive days with GF9 at indicated doses Mouse body weight (BW) was measured daily. Results are expressed as the mean±SEM (n=4 mice per group).

FIG. 72 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) suppresses tumor growth in experimental pancreatic cancer. As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GF9 (white circles), GF9-LPC (black circles) or GA/E31-LPC (half black-half white circles) at indicated doses. Treatment persisted for 31, 29 and 29 days for mice containing AsPC-1, BxPC-3 and Capan-1 tumor xenografts, respectively. Mean tumor volumes are calculated and plotted. All results are expressed as the mean±SEM (n=6 mice per group).

FIG. 73 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) is well tolerable in mice with human pancreatic cancer xenografts. As described herein, after tumors in AsPC-1-, BxPC-3- or Capan-1-bearing mice reached a volume of 150-200 mm³, mice were randomized into groups and intraperitoneally (i.p.) administered once daily 5 times per week (5qw) with either vehicle (black diamonds), GF9 (white circles), GF9-LPC (black circles) or GA/E31-LPC (half black-half white circles) at indicated doses. Treatment persisted for 31, 29 and 29 days for mice containing AsPC-1, BxPC-3 and Capan-1 tumor xenografts, respectively. Body weighs are plotted. All results are expressed as the mean±SEM (n=6 mice per group).

FIG. 74 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) suppresses tumor growth as effectively as 20 mg/kg paclitaxel and is well tolerable in mice with human non-small cell lung cancer xenografts. As described herein, after tumors in A549-bearing mice reached a volume of 150-200 mm³, mice were randomized into groups and intraperitoneally (i.p.) administered once daily 5 times per week (5qw) with either vehicle (black diamonds), paclitaxel (black squares), GF9 (white circles), GF9-LPC (black circles) or GA/E31-LPC (half black-half white circles) at indicated doses. Treatment persisted for 21 days. Mean tumor volumes are calculated and plotted. Body weighs are plotted. All results are expressed as the mean±SEM (n=6 mice per group).

FIG. 75 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) suppresses intratumoral macrophage infiltration in experimental pancreatic cancer. Intratumoral macrophage content was quantified by F4/80 staining using F4/80 antibodies. Data are shown for the groups of BxPC-3-bearing mice treated with either vehicle, GF9, GF9-LPC or GA/E31-LPC at indicated doses. Treatment persisted for 21 days. All results are expressed as the mean±SEM (n=4 mice per group). Scale bar=200 □m.

FIG. 76 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) ameliorates arthritis in mice with collagen-induced arthritis (CIA). As described herein, starting on day 24 after immunization, mice with CIA were intraperitoneally (i.p.) administered daily for 14 consecutive days with vehicle (black diamonds), dexamethasone (black squares), GF9 (white circles), GF9-LPC (black circles) and GA/E31-LPC (half black half white circles) at indicated doses. Daily clinical scores were given on a scale of 0-5 for each of the paws on days 24-38. On day 38, mice were killed and the histopathological examination of mouse joints was performed. Histopathological scores of inflammation (I), pannus (P), cartilage damage (CD), bone resorption (BR) and periosteal new bone formation (PBF) are shown. Summed histopathology scores were calculated as the sum of all five histopathological parameters. All results are expressed as the mean±SEM (n=10 mice per group).

FIG. 77 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue is well tolerable in mice with collagen-induced arthritis (CIA). Mouse body weight (BW) was measured every other day from day 24 to day 38. Mean BW changes were calculated as a percentage of the difference between beginning (day 24) and final (day 38) BWs of the CIA mice intraperitoneally (i.p.) treated daily for 14 consecutive days with vehicle, dexamethasone, GF9, GF9-LPC and GA/E31-LPC at indicated doses. All results are expressed as the mean±SEM (n=10 mice per group).

FIG. 78 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) prevents pathological appearances from collagen-induced arthritis (CIA) in mice. As described herein, toluidine blue staining of the joints from mice with CIA treated with TREM-1 inhibitory GF9 sequences or control peptide GF9-G (GFLSGSLVF) was performed. Photomicrographs of fore paws, hind paws, knees and ankles from representative mice are shown for each treatment group. For paws (original magnification 16×) and ankles (original magnification 40×), arrows identify affected joints. For knees (original magnification 100×), large arrow identifies cartilage damage, small arrow identifies pannus and arrowhead identifies bone resorption. W, wrist; S, synovium.

FIG. 79 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) reduces plasma cytokines in mice with collagen-induced arthritis (CIA). Plasma was collected on days 24, 30 and 38 from arthritic mice treated with vehicle (black diamonds), GF9 (white circles), GF9-LPC (black circles) and GA/E31-LPC (half black half white circles). Plasma samples were analyzed for concentrations of interleukin-1b (IL-1b), IL-6, and colony-stimulating factor 1 (CSF1). Results are expressed as the mean±SEM (n=5 mice per group).

FIG. 80 presents the exemplary data of one embodiment showing that treatment with free TREM-1 modulatory peptide GF9 (GFLSKSLVF) or GF9 incorporated into lipopeptide complex (GF9-LPC) or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) significantly reduces tissue expression of colony-stimulating factor 1 (CSF1) and TREM-1 in the retina of mice with oxygen-induced retinopathy (OIR) at postnatal day 17 (P17). Representative Western blots of retinal lysates from OIR mice are shown. The membrane was probed for TREM-1, reprobed for CSF1 and then for β-actin. Values in the bar graphs represent the mean±SEM, n=6. *, p<0.05, **, p<0.01 vs. vehicle-treated mice.

FIG. 81 shows exemplary illustrations of peptide GF9 blocking TREM-1 signaling by disruption of intramembrane interactions with its signaling partner, DAP-12. One example of a comparison of current approaches (upper) with a SCHOOL approach (lower), e.g. Route 1.

FIG. 82 shows exemplary illustrations of LPC delivering of peptide GF9 to macrophages, as two exemplary embodiments, e.g. each as Route 2.

FIG. 83 shows exemplary results using Pancreas Cancer: PANC-1 Xenografts demonstrating GF9 treatment inhibits tumor growth as effective as chemotherapy (Gemcitabine, GEM+nab-PTX, ABX) and Adding GF9 treatment sensitizes the tumor to chemotherapy and at least triples survival rate.

FIG. 84 shows exemplary results using Pancreas Cancer: AsPC-1 Xenografts demonstrating GF9 treatment alone does not inhibit tumor growth. Adding of the GF9 treatment sensitizes the tumor to chemotherapy. NOTE: Most tumors—abscessed.

FIG. 85 shows exemplary results using Pancreas Cancer: MiaPaca-2 Xenografts demonstrating GF9 treatment inhibits tumor growth as effective as chemotherapy (Gemcitabine, GEM+nab-PTX, ABX) and Adding of the GF9 treatment to chemo does not affect.

FIG. 86 shows exemplary results using Pancreas Cancer: BxPC-3 Xenografts demonstrating GF9 treatment inhibits tumor growth as effective as chemotherapy (Gemcitabine, GEM+nab-PTX, ABX) and Adding of the GF9 treatment to chemo does not significantly affect survival rate.

FIG. 87 shows exemplary results using Pancreas Cancer: BxPC-3 Xenografts demonstrating GF9 treatment reduces macrophage content in the tumor, Vehicle, 2.5 mg/kg GF9-LPC (5 qw, 4 wk). Shen and Sigalov, Mol Pharm 2017,14:4572, 2017.

FIG. 88 shows exemplary results using Pancreas Cancer: BxPC-3 Xenografts demonstrating GF9 treatment reduces serum cytokine levels, Vehicle, 2.5 mg/kg GF9-LPC. Shen and Sigalov, Mol Pharm 2017,14:4572, 2017.

FIG. 89 shows exemplary results using Pancreas Cancer: Xenografts demonstrating GF9 Treatment is Non-Toxic. Free GF9 tolerability (upper). GF9-LPC* tolerability (lower). * Shown for PANC-1 xenograft.

FIG. 90 shows exemplary results demonstrating that GF9 peptide is well-tolerable by healthy mice up to at least, 300 mg/kg.

FIG. 91 shows exemplary results demonstrating that in mice with collagen-induced arthritis (CIA), GF9 suppresses arthritis as effectively as dexamethasone (DEX). Study Day (Treatment: Days 24-38). I, inflammation; P, pannus; CD, cartilage damage; BR, bone resorption; PBF, periosteal new bone formation. Shen and Sigalov, Mol Pharm 2017,14:4572, 2017.

FIG. 92 shows exemplary results demonstrating that in mice with collagen-induced arthritis (CIA), GF9 treatment reduces serum IL-1b\TNFa1, IL-6 and CSF-1. Shen and Sigalov, Mol Pharm 2017,14:4572, 2017.

FIG. 93 shows exemplary results demonstrating that in mice with collagen-induced arthritis (CIA), GF9 treatment is well-tolerable: no body weight changes or other clinical symptoms are observed. Shen and Sigalov, Mol Pharm 2017,14:4572, 2017.

FIG. 94 shows exemplary results demonstrating that in NSCLC: A549 Xenografts, GF9 inhibits tumor growth as effectively as chemo (20 mg/kg Paclitaxel, PTX). Sigalov, 2014, 21:208.

FIG. 95 shows exemplary results demonstrating that in Capan-1 xenografts, GF9 inhibits tumor growth and reduces serum cytokines, including CSF-1 (but not VEGF). Shen and Sigalov, Mol Pharm 2017,14:4572, 2017.

FIG. 96 shows exemplary results demonstrating that GF9 is well-tolerated by long term treated cancer mice in Capan-1 Xenografts and A549 Xenografts, GF9 inhibits tumor growth as effectively as chemo (20 mg/kg Paclitaxel, PTX). Sigalov, 2014, 21:208.

FIG. 97A-C shows exemplary current approaches for blocking TREM-1 binding to its uncertain ligand (Bouchon et al. 2001, Schenk et al. 2007, Gibot et al. 2008, Gibot et al. 2009, Murakami et al. 2009, Luo et al. 2010, Derive et al. 2013, Derive et al. 2014) FIG. 97A In contrast, GF9 self-penetrates into the membrane and disrupts TREM-1/DAP12 interactions FIG. 97B when colocalizes with TREM-1 FIG. 97C. FIG. 97A. CURRENT. FIG. 97B. SCHOOL FIG. 97C. CONFOCAL.

FIG. 98A-B shows exemplary results demonstrating that GF9 is non-toxic in healthy mice FIG. 98A and reduces TREM-1 and M-CSF overexpression in the retina of mice with oxygen-induced retinopathy FIG. 98B. FIG. 98A Graph. FIG. 98B Blot.

FIG. 99A-C shows exemplary results demonstrating that Oxidized apo A-I peptides in LPC increase J774 intracellular uptake of GF9-LPC in vitro FIG. 99A, 99B and enable in vivo delivery to macrophages FIG. 99C (as shown using magnetic resonance imaging (MRI) and confocal microscopy (Sigalov 2014, Sigalov 2014, Shen and Sigalov 2017)). FIG. 99A. IN VITRO. FIG. 99B. CONFOCAL red: Rho B-PE; green: 488-GF9; blue: 405-apo A-I PE22. FIG. 99C. MOUSE AORTA.

FIG. 100A-D shows exemplary results demonstrating that GF9-dLPC (disks) and GF9-sLPC (spheres) reduce LPS-induced cytokine release in vitro FIG. 100A and in vivo FIG. 100B and prolong survival FIG. 100C (Sigalov 2014). In cancer mice, GF9 and GF9-LPC treatments inhibit production of CSF-1/M-CSF but not VEGF FIG. 100D (Shen and Sigalov 2017). FIG. 100A. CYTOKINES IN VITRO. FIG. 100B. CYTOKINES IN VIVO. FIG. 100C. SURVIVAL IN LPS-INDUCED SEPTIC MICE. FIG. 100D. M-CSF/VEGF RELEASE IN CANCER MICE.

FIG. 101 shows exemplary results demonstrating that Different rate and efficiency of GF9-dLPC and GF9-sLPC in vitro uptake by J774 macrophages (Sigalov 2014).

FIG. 102 shows exemplary results demonstrating that Stability of GF9-LPC. GF9-LPC AT 4° C.

FIG. 103A-D shows exemplary results demonstrating that GF9-LPC daily i.p. administered at 2.5 mg/kg suppress the expression of TREM-1, MCP-1/CCL2 and early fibrosis marker molecules in mice with ALD. Indicates significance level compared to nontreated pair-fed group; #indicates significance level compared to nontreated alcohol-fed group. Significance levels are as follows: *, p<0.05; **^{/##}, p<0.01; ***, p<0.001; ****^{/####}, p<0.0001. FIG. 102A. TREM-1. FIG. 102B. MCP-1/CCL2. FIG. 102C. Pro-Coll1alpha. FIG. 102D. alpha-SMA.

FIG. 104A-D shows exemplary results demonstrating that GF9 and GF9-LPC daily i.p. administered are well-tolerated FIG. 104A, suppress macrophage infiltration into the tumor FIG. 104B, 104C and inhibit release of CSF-1/M-CSF but not VEGF FIG. 104D. Scale bar=200 μm. *, p<0.05; **, p<0.01; ***, p<0.001; ****, p<0.0001 (vs vehicle). FIG. 104A. BODY WEIGHT. FIG. 104B. INTRATUMORAL MACROPHAGE INFILTRATION—INHIBITION BY GF9 AND GF9-LPC. FIG. 104C. MACROPHAGE INFILTRATION. FIG. 104D. M-CSF/VEGF RELEASE IN CANCER MICE.

FIG. 105A-C shows exemplary results demonstrating that in mice with autoimmune arthritis, GF9, discoidal GF9-LPC (GF9-dHDL) and spherical GF9-LPC (GF9-sHDL) i.p. administered daily are well-tolerated FIG. A, ameliorate the disease FIG. 105B and inhibit production of cytokines and M-CSF FIG. C (Shen and Sigalov 2017). FIG. 105A. BODY WEIGHT CHANGES. FIG. 105B. ARTHRITIS AMELIORATION. FIG. 105C. CYTOKINE RELEASE IN ARTHRITIC MICE.

### DEFINITIONS

The term, “composition”, as used herein, refers to any mixture of substances comprising a peptide and/or compound contemplated by the present invention. Such a composition may include the substances individually or in any combination.

As used herein the term “lipoprotein” such as VLDL (very low density lipoproteins), LDL (low density lipoproteins) and HDL (high density lipoproteins), refers to lipoproteins found in the serum, plasma and lymph, in vivo, related to lipid transport. The chemical composition of each lipoprotein differs, for examples, HDL has a higher proportion of protein versus lipid, whereas the VLDL has a lower proportion of protein versus lipid. When referring to lipoproteins, the term “native” refers to naturally-occurring (e.g., a “wild-type”) lipoproteins.

The terms “APOA1_HUMAN”, “Apolipoprotein A-I”, “Apolipoprotein A-1”, “APOA1”, “ApoA-I”, “Apo-AI”, “ApoA-1”, “apo-A1”, “apoA-1” and “Apo-A1” refer to the naturally occurring human protein listed in the UniProt Knowledgebase (UniProtKB, www.uniprot.org) under the name “APOA1_HUMAN”. The protein amino acid sequence can be found under the entry UniProt KB/Swiss-Prot P02647 (www.uniprot.org/uniprot/P02647). The terms “APOA2_HUMAN”, “Apolipoprotein A-II”, Apolipoprotein A-2”, “APOA2”, “ApoA-II”, “Apo-AII”, “ApoA-2”, “apo-A2”, “apoA-2” and “Apo-A2” refer to the naturally occurring human protein listed in the UniProt Knowledgebase (UniProtKB, www.uniprot.org) under the name “APOA2_HUMAN”. The protein amino acid sequence can be found under the entry UniProt KB/Swiss-Prot P02652 (http://www.uniprot.org/uniprot/P02652).

The term “TREM receptor”, as used herein, refers to a member of TREM receptor family including: TREM-1, TREM-2, TREM-3 and TREM-4. The terms “TREM1_HUMAN”, “TREM-1 receptor”, “TREM-1 receptor subunit”, “TREM-1 subunit”, and “TREM-1 recognition subunit” refer to the naturally occurring human protein listed in the UniProt Knowledgebase (UniProtKB, www.uniprot.org) under the name “TREM1_HUMAN”. The protein amino acid sequence can be found under the entry UniProt KB/Swiss-Prot Q9NP99.

The term “TREM receptor”, as used herein, refers to a member of TREM receptor family: TREM-1, TREM-2, TREM-3 and TREM-4.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (p) chain, although in some cells the TCR consists of gamma and delta (y/S) chains. TCRs may exist in alpha/beta and gamma/delta forms, which are structurally similar but have distinct anatomical locations and functions. Each chain is composed of two extracellular domains, a variable and constant domain, in some embodiments, the TCR may be modified on any ceil comprising a TCR, including, for example, a helper T cell, a cytotoxic T cell, a memory T ceil, regulatory T cell, natural killer T cell, and gamma delta T cell.

As employed herein and understood by the ordinary skill in the art, “amino acid domain” is a contiguous polymer of at least 2 amino acids joined by peptide bond(s). The domain may be joined to another amino acid or amino acid domain by one or more peptide bonds. An amino acid domain can constitute at least two amino acids at the N-terminus or C-terminus of a peptide or can constitute at least two amino acids in the middle of a peptide.

The term “antibody” herein refers to a protein, derived from a germline immunoglobulin sequence, which is capable of specifically binding to an antigen (TREM-1) or a portion thereof. The term includes full length antibodies of any class or isotype (that is, IgA, IgE, IgG, IgM and/or IgY) and any single chain or fragment thereof. An antibody that specifically binds to an antigen, or portion thereof, may bind exclusively to that antigen, or portion thereof, or it may bind to a limited number of homologous antigens, or portions thereof.

As used herein, a “peptide” and “polypeptide” comprises a string of at least two amino acids linked together by peptide bonds. A peptide generally represents a string of between approximately 2 and 200 amino acids, more typically between approximately 6 and 64 amino acids. Peptide may refer to an individual peptide or a collection of peptides. Inventive peptides typically contain natural amino acids, although non-natural amino acids (i.e., compounds that do not occur in nature but that can be incorporated into a polypeptide chain and/or amino acid analogs as are known in the art may alternatively be employed. In particular, D-amino acids may be used.

As employed herein and understood by the ordinary skill in the art, “peptide sequence”, or “amino acid sequence”, is the order in which amino acid residues, connected by peptide bonds, lie in the chain in peptides. The sequence is generally reported from the N-terminal end containing free amino group to the C-terminal end containing free carboxyl group. “Peptide sequence” is often called “protein sequence” if it represents the primary structure of a protein (http://en.wikipedia.org/wiki/Peptide_sequence).

Peptides and compositions of the present invention made synthetically may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Naturally occurring residues are divided into groups based on common side chain properties: as described herein. Analogues may be generated by substitutional mutagenesis and retain the biological activity of the original trifunctional peptides. Examples of substitutions identified as “conservative substitutions” are shown in TABLE 1. If such substitutions result in a change not desired, then other type of substitutions, denominated “exemplary substitutions” in TABLE 1, or as further described herein in reference to amino acid classes, are introduced and the products screened for their capability of executing three functions.

The term “amphipathic” is used herein to describe a molecule that has both polar and non-polar parts and as such, has two different affinities, as a polar end that is attracted to water and a nonpolar end that is repelled by it. An amphipathic helix is defined as an alpha helix with opposing polar and nonpolar faces oriented along the long axis of the helix. As well known in the art, amino acid sequences can be screened for amphipathic helixes and an amphipathicity score can be calculated using a variety of computer programs available online (see, for example, http://www.tcdb.org/progs/?tool=pepwheel, http://lbqp.unb.br/NetWheels/, https://npsa-prabi.ibcp.fr/cgi-bin/npsa_automat.pl?page=/NPSA/npsa_amphipaseek.html, http://rzlab.ucr.edu/scripts/wheel/wheel.cgi, http://heliquest.ipmc.cnrs.fr/cgi-bin/ComputParams.py) or other techniques including but not limiting to those described in Jones, et al. J Lipid Res 1992, 33:287-296.

As used herein, the term “aptamer” or “specifically binding oligonucleotide” refers to an oligonucleotide that is capable of forming a complex with an intended target substance.

In the present disclosure, the term “modified peptide” is used to describe chemically or enzymatically, or chemically and enzymatically modified oligopeptides, oligopseudopeptides, polypeptides, and pseudopolypeptides (synthetic or otherwise derived), regardless of the nature of the chemical and/or enzymatic modification. The term “pseudopeptide” refers to a peptide where one or more peptide bonds are replaced by non-amido bonds such as ester or one or more amino acids are replaced by amino acid analogs. The term “peptides” refers not only to those comprised of all natural amino acids, but also to those which contain unnatural amino acids or other non-coded structural units. The terms “peptides”, when used alone, include pseudopeptides. It is worth mentioning that “modified peptides” have utility in many biomedical applications because of their increased stability against in vivo degradation, superior pharmacokinetics, and altered immunogenicity compared to their native counterparts.

The term “modified peptides,” as employed herein, also includes oxidized peptides.

The term “oxidized peptide” refers to a peptide in which at least one amino acid residue is oxidized.

The term “analog”, as used herein, includes any peptide having an amino acid sequence substantially identical to one of the sequences specifically shown herein in which one or more residues have been conservatively substituted with a functionally similar residue and which displays the abilities as described herein. Examples of conservative substitutions include the substitution of one non-polar (hydrophobic) residue such as isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another.

The term “conservative substitution”, as used herein, also includes the use of a chemically derivatized residue in place of a non-derivatized residue provided that such peptide displays the requisite inhibitory function on myeloid cells as specified herein. The term derivative includes any chemical derivative of the peptide of the invention having one or more residues chemically derivatized by reaction of side chains or functional groups.

The term “homolog” or “homologous” when used in reference to a polypeptide refers to a high degree of sequence identity between two polypeptides, or to a high degree of similarity between the three-dimensional structures or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

As applied to polypeptides, the term “substantial identity” means that two peptide sequences, when optimally aligned, such as, for example, by the programs KALIGN, DOTLET, LALIGN and DIALIGN (https://www.expasy.org/tools) using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The term “modified peptides,” as employed herein, also includes oxidized peptides. The term “oxidized peptide” refers to a peptide in which at least one amino acid residue is oxidized. The term “oxidation status” refers to a metric of the extent to which specific amino acid residues are replaced by corresponding oxidized amino acid residues in a peptide. The term “extent of oxidation” refers to the degree to which potentially oxidizable amino acids in a peptide have undergone oxidation. For example, if the peptide contains a single tyrosine residue which is potentially oxidized to 3-chlorotyrosine, then an increase in mass of about 34 Dalton (i.e., the approximate difference in mass between chlorine and hydrogen) indicates oxidation of tyrosine to 3-chlorotyrosine. Similarly, if the peptide contains a single methionine residue which is potentially oxidized to methionine sulfoxide, then an increase in mass of 16 Dalton (i.e., the difference in mass between methionine and methionine containing one extra oxygen) indicates oxidation of methionine to methionine sulfoxides.

The term “oxidation status” refers to a metric of the extent to which specific amino acid residues are replaced by corresponding oxidized amino acid residues in a peptide. The term “extent of oxidation” refers to the degree to which potentially oxidizable amino acids in a peptide have undergone oxidation. For example, if the peptide contains a single tyrosine residue which is potentially oxidized to 3-chlorotyrosine, then an increase in mass of about 34 Dalton (i.e., the approximate difference in mass between chlorine and hydrogen) indicates oxidation of tyrosine to 3-chlorotyrosine. Similarly, if the peptide contains a single methionine residue which is potentially oxidized to methionine sulfoxide, then an increase in mass of 16 Dalton (i.e., the difference in mass between methionine and methionine containing one extra oxygen) indicates oxidation of methionine to methionine sulfoxides.

The oxidation status can be measured by metrics known to the arts of protein and peptide chemistry (as disclosed in Caulfield, U.S. Pat. No. 8,114,613 and Hazen, et al., U.S. Pat. No. 8,338,110, herein incorporated by reference) including, without limitation, assay of the number of oxidized residues, mass spectral peak intensity, mass spectral integrated area, and the like. In some embodiments, oxidation status is reported as a percentage, wherein 0% refers to no oxidation and 100% refers to complete oxidation of potentially oxidizable amino acid residues within apo A-I or apo A-II peptide fragments.

The term “potentially subject to oxidation,” “potentially oxidizable amino acid residues”, and the like refer to an amino acid which can undergo oxidation, for example by nitration or chlorination.

A “biologically active peptide motif” is a peptide that induces a phenotypic response or change in an appropriate cell type when the cell is contacted with the peptide. The peptide may be present either in isolated form or as part of a larger polypeptide or other molecule. The ability of the peptide to elicit the response may be determined, for example, by comparing the relevant parameter in the absence of the peptide (e.g., by mutating or removing the peptide when normally present within a larger polypeptide). Phenotypic responses or changes include, but are not limited to, enhancement of cell spreading, attachment, adhesion, proliferation, secretion of an extracellular matrix (ECM) molecule, or expression of a phenotype characteristic of a particular differentiated cell type.

As used herein, a “minimal biologically active sequence” refers to the minimum length of a sequence of a peptide that has a specific biological function. In a first example, -IVILLAGGFLSKSLVFSVLFA- (e.g., Domain A, SEQ ID NO. 47) is a biologically active TREM-1 inhibitory sequence corresponding to the human TREM-1 transmembrane domain, wherein -GFLSKSLVF- (e.g. Domain A, SEQ ID NO. 1) has the sole function of TREM-1 inhibition. Thus, in this case, -GFLSKSLVF- (Domain A, SEQ ID NO. 1) is a “minimal biologically active sequence.” In a second example, the sequence -PLGEEMRDRARAHVDALRTHLARGD, and an internal sequence -GEEMRDRARAHVRGD- (Domain B, SEQ ID NO. 5) contains the sequence -RGD-; -RGD- has a cell attachment function.

However, -PLGEEMRDRARAHVDALRTHLARGD and -GEEMRDRARAHVRGD- (Domain B, SEQ ID NO. 5) also functions to assist in the formation of naturally long half-life lipopeptide/lipoprotein particles upon interaction with native lipoproteins and to promote binding of these particles with scavenger receptor type I (SR-B1). Thus, in this case, both -PLGEEMRDRARAHVDALRTHLARGD-andGEEMRDRARAHVRGD-(Domain B, SEQ ID NO. 5) in addition to -RGD- are considered a “minimal biologically active sequence.” In another example, the sequence -GFLSKSLVFPLGEEMRDRARAHVDALRTHLARGD- (SEQ ID NO. . . . ) contains the sequence -RGD-; -RGD- has a cell attachment function. However, -GFLSKSLVFPLGEEMRDRARAHVDALRTHLARGD- (SEQ ID NO. . . . ) also has the functions of inhibition of TREM-1, assistance in the self-assembly of naturally long half-life lipopeptide particles upon binding to lipid or lipid mixtures particle and of interaction with scavenger receptor type I (SRBI). Thus, in this case, both -GFLSKSLVFPLGEEMRDRARAHVDALRTHLARGD- (SEQ ID NO. . . . ) and -RGD- are considered a “minimal biologically active sequence.” As is understood from the present invention, the first and second amino acid domains of a resulting peptide contain at least one minimal biologically active sequence. This minimal biologically active sequence is any length of sequence from an original peptide sequence. Moreover, with the exception of the amino acids of the minimal biologically active sequence, the amino acids of any or both amino acid domain can be exchanged, added or removed according to the design of the molecule to adjust its overall hydrophilicity and/or net charge. In certain embodiments, the minimal biologically active sequence refers to any one of the sequences provided in TABLE 2.

The term “imaging agent” or “imaging probe” as used herein refers to contrast agents used in imaging techniques such as computed tomography (CT), gamma-scintigraphy, positron emission tomography (PET), single photon emission computed tomography (SPECT), magnetic resonance imaging (MRI), and combined imaging techniques in order to improve diagnostic performance of medical imaging.

The term “labeling substance or label or labeled probe” refers to a substance that can image whether there is a binding between the modulator and the cellular component (e.g., TREM-1/DAP-12 receptor complex), and can visualize the binding by a pattern. It may include radioactive materials, fluorescent or emitting materials.

The term “carrier” as used herein, refers to a biocompatible nanoparticle that facilitates administration of a pharmaceutical agent to an individual.

The term “encapsulation” as used herein refers to the enclosure of a molecule, such as trifunctional peptides and compounds of the present invention, inside the nanoparticle. The term “incorporation” as used herein refers to imbibing or adsorbing the trifunctional peptides and compounds onto the nanoparticle. The terms “reconstituted” and “recombinant” as used herein both refer to synthetic lipopeptide particles that represent both discoidal and spherical nanoparticles and mimic native HDL particles.

As used herein, “naturally occurring” means found in nature. A naturally occurring biomolecule is, in general, synthesized by an organism that is found in nature and is unmodified by the hand of man, or is a degradation product of such a molecule. A molecule that is synthesized by a process that involves the hand of man (e.g., through chemical synthesis not involving a living organism or through a process that involves a living organism that has been manipulated by the hand of man or is descended from such an organism) but that is identical to a molecule that is synthesized by an organism that is found in nature and is unmodified by the hand of man is also considered a naturally occurring molecule.

A “site of interest” on a target as used herein is a site to which modified peptides and compounds of the present invention bind.

The term “target site”, as used herein, refers to sites/tissue areas of interest.

As used in this invention, the terms “target cells” or “target tissues” refer to those cells or tissues, respectively that are intended to be targeted using the compositions of the present invention delivered in accord with the invention. Target cells or target tissues take up or link with the modified peptides and compounds of the invention. As used in this invention, the terms “target cells” or “target tissues” refer to those cells or tissues, respectively that are intended to be treated and/or visualized in imaging techniques such as CT, gamma-scintigraphy, PET, SPECT, MRI, and combined imaging techniques, using the compositions of the present invention delivered in accord with the invention. Target cells are cells in target tissue, and the target tissue includes, but is not limited to, atherosclerotic plaques, vascular endothelial tissue, abnormal vascular walls of tumors, solid tumors, tumor-associated macrophages, and other tissues or cells related to cancer, cardiovascular, inflammatory, autoimmune diseases, and the like. Further, target cells include virus-containing cells, and parasite-containing cells. Also included among target cells are cells undergoing substantially more rapid division as compared to non-target cells.

The term “target cells” also includes, but is not limited to, microorganisms such as bacteria, viruses, fungi, parasites, and infectious agents. Thus, the term “target cell” is not limited to living cells but also includes infectious organic particles such as viruses. “Target compositions” or “target biological components” include, but are not be limited to: toxins, peptides, polymers, and other compounds that may be selectively and specifically identified as an organic target that is intended to be visualized in imaging techniques using the compositions of the present invention.

The term “therapeutic agent” or “drug” as used herein refers to any compound or composition having preventive, therapeutic or diagnostic activity, primarily but not exclusively in the treatment of patients with macrophage (myeloid cell)-related diseases. The term “myeloid cells” include monocytes, macrophages, neutrophils, basophils, eosinophils, erythrocytes, and megakaryocytes to platelets.

The terms “macrophage-associated”, “macrophage-mediated”, and “macrophage-related diseases” include diseases associated with macrophages as disclosed in Low and Turk, U.S. Pat. No. 8,916,167, herein incorporated by reference in its entirety.

The term “plaque” includes, for example, an atherosclerotic plaque.

The term “myeloid cell-mediated pathology” (or “myeloid cell-related pathologies”, or “myeloid cell-mediated disorder, or “myeloid cell-related disease”), as used herein, refers to any condition in which an inappropriate myeloid cell response is a component of the pathology. The term is intended to include both diseases directly mediated by myeloid cells, and also diseases in which an inappropriate myeloid cell response contributes to the production of abnormal antibodies, antibodies, as well as graft rejection.

The term “ligand-induced myeloid cell activation”, as used herein, refers to myeloid cell activation in response to the stimulation by the specific ligand.

The term “stimulation”, as used herein, refers to a primary response induced by ligation of a cell surface moiety. For example, in the context of receptors, such stimulation entails the ligation of a receptor and a subsequent signal transduction event. With respect to stimulation of a myeloid cell, such stimulation refers to the ligation of a myeloid cell surface moiety that in one embodiment subsequently induces a signal transduction event, such as binding the TREM-1/DAP-12 complex. Further, the stimulation event may activate a cell and up-regulate or down-regulate expression or secretion of a molecule.

The term “ligand”, or “antigen”, as used herein, refers to a stimulating molecule that binds to a defined population of cells. The ligand may bind any cell surface moiety, such as a receptor, an antigenic determinant, or other binding site present on the target cell population. The ligand may be a protein, peptide, antibody and antibody fragments thereof, fusion proteins, synthetic molecule, an organic molecule (e.g., a small molecule), or the like. Within the specification and in the context of myeloid cell stimulation, the ligand (or antigen) binds the TREM receptor and this binding activates the myeloid cell.

The term “activation”, as used herein, refers to the state of a cell following sufficient cell surface moiety ligation to induce a noticeable biochemical or morphological change. Within the context of myeloid cells, such activation, refers to the state of a myeloid cell that has been sufficiently stimulated to induce production of interleukin (TL) 1, 6 and/or 8 (IL-1, IL-6 and/or IL-8, respectively) and tumor necrosis factor alpha (TNF-alpha), differentiation of primary monocytes into immature dendritic cells, and enhancement of inflammatory responses to microbial products. Within the context of other cells, this term infers either up or down regulation of a particular physico-chemical process.

The term “inhibiting myeloid cell activation” (or “TREM-mediated cell activation”), as used herein, refers to the slowing of myeloid cell activation, as well as completely eliminating and/or preventing myeloid cell activation.

The term, “treating a disease or condition”, as used herein, refers to modulating myeloid cell activation including, but not limited to, decreasing cytokine production and differentiation of primary monocytes into immature dendritic cells and/or slowing myeloid cell activation, as well as completely eliminating and/or preventing myeloid cell activation. Myeloid cell-related diseases and/or conditions treatable by modulating myeloid cell activation include, but are not limited to, cancer including but not limited to lung cancer, pancreatic cancer, multiple myeloma, melanoma, leukemia, prostate cancer, breast cancer, liver cancer, bladder cancer, stomach cancer, prostate cancer, colon cancer, colorectal cancer, CNS cancer, melanoma, ovarian cancer, gastrointestinal cancer, renal cancer, or osteosarcoma and other cancers, brain and skin cancers, endometrial cancer, esophageal cancer, kidney cancer, thyroid cancer, neuroblastoma, neurofibroma, glioma, glioblastoma, glioblastoma multiforme, head and neck cancer, cervical cancer, giant cell tumor of the tendon sheath (GCTTS), tenosynovial giant cell tumor (TGCT; also referred to in the art as TSGCT), PVNS and other cancers in which myeloid cells are involved or recruited, cancer cachexia, in addition to ALD, atherosclerosis, allergic diseases, acute radiation syndrome, inflammatory bowel disease, empyema, alcohol-induced liver disease, nonalcoholic fatty liver disease and non-alcoholic steatohepatitis, acute mesenteric ischemia, hemorrhagic shock, multiple sclerosis, autoimmune diseases, including but not limited to, atopic dermatitis, lupus, scleroderma, rheumatoid arthritis, psoriatic arthritis and other rheumatic diseases, sepsis, diabetic retinopathy and retinopathy of prematurity, Alzheimer's, Parkinson's and Huntington's diseases, and other myeloid cell-related inflammatory conditions eg myositis, tissue/organ rejection, brain and spinal cord injuries. Other exemplary cancers include, but are not limited to, adrenocortical carcinoma, acquired immune deficiency syndrome (AIDS)-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, bronchial adenomas/carcinoids, carcinoid tumor, nervous system cancer, nervous system lymphoma, central nervous system lymphoma, cervical cancer, childhood cancers, chronic lymphocytic leukemia, chronic myelogenous leukemia, chronic myeloproliferative disorders, cutaneous T-cell lymphoma, lymphoid neoplasm, mycosis fungoides, Seziary Syndrome, esophageal cancer, extracranial germ cell tumor, extragonadal germ cell tumor, extrahepatic bile duct cancer, eye cancer, intraocular melanoma, retinoblastoma, gallbladder cancer, gastrointestinal carcinoid tumor, gastrointestinal stromal tumor (GIST), germ cell tumor, ovarian germ cell tumor, gestational trophoblastic tumor glioma, Hodgkin lymphoma, hypopharyngeal cancer, intraocular melanoma, ocular cancer, islet cell tumors (endocrine pancreas), Kaposi Sarcoma, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian epithelial cancer, ovarian low malignant potential tumor, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

The term “detectable” refers to the ability to detect a signal over the background signal.

In accordance with the present disclosure, “a detectably effective amount” of the labeled probe of the present disclosure is defined as an amount sufficient to yield an acceptable image using equipment that is available for clinical use. A detectably effective amount of the labeled probe of the present disclosure may be administered in more than one injection. The detectably effective amount of the labeled probe of the present disclosure can vary according to factors such as the degree of susceptibility of the individual, the age, sex, and weight of the individual, idiosyncratic responses of the individual, and the like.

Detectably effective amounts of the probe of the present disclosure can also vary according to instrument and film-related factors. Optimization of such factors is well within the level of skill in the art.

The term “in vivo imaging” as used herein refers to methods or processes in which the structural, functional, molecular, or physiological state of a living being is examinable without the need for a life-ending sacrifice.

The term “inhibiting T cell activation”, as used herein, refers to the slowing of T cell activation, as well as completely eliminating and/or preventing T cell activation.

The term “T cell-mediated pathology” (or “T cell-related pathologies”, or “T cell-mediated disorder, or “T cell-related disease”), as used herein, refers to any condition in which an inappropriate T cell response is a component of the pathology. The term is intended to include both diseases directly mediated by T cells, and also diseases in which an inappropriate T cell response contributes to the production of abnormal antibodies, as well as graft rejection.

The term “treating a T cell-mediated disease or condition”, as used herein, refers to modulating T cell activation including, but not limited to, decreasing cellular proliferation, cytokine production and performance of regulatory or cytolytic effector functions and/or slowing T cell activation, as well as completely eliminating and/or preventing T cell activation. T cell-related diseases and/or conditions treatable by modulating T cell activation include, but are not limited to, systemic lupus erythematosus, rheumatoid arthritis, psoriatic arthritis, multiple sclerosis, type I diabetes, gastroenterological conditions e.g. inflammatory bowel disease, Crohn's disease, celiac, Guillain-Barre syndrome, Hashimotos disease, pernicious anaemia, primary biliary cirrhosis, chronic active hepatitis; skin problems e.g. atopic dermatitis, psoriasis, pemphigus vulgaris; cardiovascular problems e.g. autoimmune pericarditis, allergic diathesis e.g. delayed type hypersensitivity, contact dermatitis, AIDS virus, herpes simplex/zoster, respiratory conditions e.g. allergic alveolitis, inflammatory conditions e.g. myositis, ankylosing spondylitis, tissue/organ rejection.

The term, “subject” or “patient”, as used herein, refers to any individual organism. For example, the organism may be a mammal, such as a primate (i.e., for example, a human) or a laboratory animal. Further, the organism may be a domesticated animal (i.e., for example, cats, dogs, etc.), livestock (i.e., for example, cattle, horses, pigs, sheep, goats, etc.), or a laboratory animal (i.e., for example, mouse, rabbit, rat, guinea pig, etc.).

The term, “therapeutically effective amount”, “therapeutically effective dose” or “effective amount”, as used herein, refers to an amount needed to achieve a desired clinical result or results (e.g. inhibiting receptor-mediated cell activation) based upon trained medical observation and/or quantitative test results. The potency of any administered peptide or compound determines the “effective amount” which can vary for the various compounds that inhibit myeloid cell activation (i.e., for example, compounds inhibiting TREM ligand-induced myeloid cell activation and/or TCR-mediated T cell activation). Additionally, the “effective amount” of a compound may vary depending on the desired result, for example, the level of myeloid cell activation inhibition desired. The “therapeutically effective amount” necessary for inhibiting differentiation of primary monocytes into immature dendritic cells may differ from the “therapeutically effective amount” necessary for preventing or inhibiting cytokine production.

The term, “agent”, as used herein, refers to any natural or synthetic compound (i.e., for example, a peptide, a peptide variant, or a small molecule).

The term, “intrinsic helicity”, as used herein, refers to the helicity which is adopted by a peptide in an aqueous solution. The term, “induced helicity”, as used herein, refers to the helicity which is adopted by a peptide when in the presence of a helicity inducer, including, but not limited to, trifluoroethanol (TFE), detergents (e.g., sodium dodecyl sulfate, SDS) or lipids.

The term “therapeutic drug”, as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars. Drugs or compounds may have any of a variety of activities, which may be stimulatory or inhibitory, such as antibiotic activity, antiviral activity, antifungal activity, steroidal activity, cytotoxic, cytostatic, anti-proliferative, anti-inflammatory, analgesic or anesthetic activity, or can be useful as contrast or other diagnostic agents.

The term “effective dose” as used herein refers to the concentration of any compound or drug contemplated herein that results in a favorable clinical response. In solution, an effective dose may range between approximately 1 ng/ml and 100 mg/ml, preferably between 100 ng/ml and 10 mg/ml, but more preferably between 500 ng/ml and 1 mg/ml.

The term “effective amount” or “therapeutically effective amount” refers to an amount of a drug effective to treat a disease or disorder in a subject. In certain embodiments, an effective amount refers to an amount effective, at dosages and for periods of time necessary, to achieve the desired therapeutic or prophylactic result. A therapeutically effective amount of the compound or composition of the invention that modulate TREM-1/DAP-12 receptor complex signaling may vary according to factors such as the disease state, age, sex, and weight of the individual, and the ability of the compound or composition to elicit a desired response in the individual. A therapeutically effective amount encompasses an amount in which any toxic or detrimental effects of the compound or composition are outweighed by the therapeutically beneficial effects. As one example, in some embodiments, the expression “effective amount” refers to an amount of the compound or composition that is effective for treating cancer.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “induction therapy” refers to the first treatment given for a disease. It is often part of a standard set of treatments, such as surgery followed by chemotherapy and radiation. When used by itself, induction therapy is the one accepted as the best treatment. If it doesn't cure the disease or it causes severe side effects, other treatment may be added or used instead. Also called first-line therapy, primary therapy, and primary treatment.

A “maintenance therapy” refers to a medical therapy that is designed to help a primary treatment succeed. For example, maintenance chemotherapy may be given to people who have a cancer in remission in an attempt to prevent a relapse. In other words, treatment that is given to help keep cancer from coming back after it has disappeared following the initial therapy. It may include treatment with drugs, vaccines, or antibodies that kill cancer cells or keep tumor unfavorable microenvironment, and it may be given for a long time. This form of treatment is also a common approach for the management of many incurable, chronic diseases such as periodontal disease, Crohn's disease or ulcerative colitis.

Administration “in combination with” one or more further therapeutic agents includes simultaneous (concurrent) and consecutive (sequential) administration in any order.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The term “administered” or “administering” a drug or compound, as used herein, refers to any method of providing a drug or compound to a patient such that the drug or compound has its intended effect on the patient. For example, one method of administering is by an indirect mechanism using a medical device such as, but not limited to a catheter, syringe etc. A second exemplary method of administering is by a direct mechanism such as, local tissue administration (i.e., for example, extravascular placement), oral ingestion, transdermal patch, topical, inhalation, suppository etc.) The term, “agent”, as used herein, refers to any natural or synthetic compound (i.e., for example, a peptide, a peptide variant, or a small molecule).

The term, “composition”, as used herein, refers to any mixture of substances comprising a peptide and/or compound contemplated by the present invention. Such a composition may include the substances individually or in any combination.

The term “modulator” used in this invention refers to a substance and/or compositions contemplated by the present invention or a combination thereof with capacity to inhibit (e.g., “antagonist” activity) a functional property of biological activity or process (e.g., reducing or blocking TREM-1/DAP-12 activity—signaling and/or activation); such inhibition can be contingent on the occurrence of a specific event, such as reduction or blockade of a signal transduction pathway, and/or can be manifest only in particular cell types. For instance, small molecules such as drugs, proteins such as antibodies, hormones or growth factors, protein domains, protein motifs, and peptides or a combination thereof can act as a modulator.

The term “tissue sample” refers to a collection of similar cells obtained from a tissue of a subject. The source of the tissue sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, synovial fluid, or interstitial fluid; cells from any time in gestation or development of the subject. In some embodiments, a tissue sample is a synovial biopsy tissue sample and/or a synovial fluid sample. In some embodiments, a tissue sample is a synovial fluid sample. The tissue sample may also be primary or cultured cells or cell lines. Optionally, the tissue sample is obtained from a disease tissue/organ. The tissue sample may contain compounds that are not naturally intermixed with the tissue in nature such as preservatives, anticoagulants, buffers, fixatives, nutrients, antibiotics, or the like. A “control sample” or “control tissue”, as used herein, refers to a sample, cell, or tissue obtained from a source known, or believed, not to be afflicted with the disease for which the subject is being treated.

For the purposes herein a “section” of a tissue sample means a part or piece of a tissue sample, such as a thin slice of tissue or cells cut from a solid tissue sample.

The term “anti-inflammatory drug” means any compound, composition, or drug useful for preventing or treating inflammatory disease.

The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a drug or compound to a patient during a medical procedure or therapy is contemplated herein as a medical device. “Direct medical implants” include, but are not limited to, urinary and intravascular catheters, dialysis catheters, wound drain tubes, skin sutures, vascular grafts and implantable meshes, intraocular devices, implantable drug delivery systems and heart valves, and the like. “Wound care devices” include, but are not limited to, general wound dressings, non-adherent dressings, burn dressings, biological graft materials, tape closures and dressings, surgical drapes, sponges and absorbable hemostats. “Surgical devices” include, but are not limited to, surgical instruments, endoscope systems (i.e., catheters, vascular catheters, surgical tools such as scalpels, retractors, and the like) and temporary drug delivery devices such as drug ports, injection needles etc. to administer the medium. A medical device is “coated” when a medium comprising an anti-inflammatory drug (i.e., for example, the peptides, compositions, and compounds of the present invention) becomes attached to the surface of the medical device. This attachment may be permanent or temporary. When temporary, the attachment may result in a controlled release of an inflammatory drug.

### DETAILED DESCRIPTION OF THE INVENTION

The invention disclosed herein provides compositions and methods of treating cancer and other diseases related to activated immune cells using modulators of the TREM-1/DAP-12 signaling pathway. The compositions, including peptides and peptide variants, modulate TREM-1-mediated immunological response as standalone and combination-therapy treatment regimen. Further, methods are provided for predicting the efficacy of TREM-1 modulatory therapies in patients. In one embodiment, the present invention relates to targeted treatment, prevention and/or detection of cancer including but not limited to lung cancer including non-small cell lung cancer, pancreatic cancer, giant cell tumor of the tendon sheath, tenosynovial giant cell tumor, pigmented villonodular synovitis, cancer cachexia, etc., and other cancers associated with myeloid cell activation and recruitment. Additionally, the present invention relates to the targeted treatment, prevention and/or detection of scleroderma including but not limited to calcinosis, Raynaud's phenomenon, esophageal dysmotility, scleroderma, or telangiectasia syndrome (CREST). The invention further relates to personalized medical treatments.

The present disclosure describes novel amphipathic trifunctional peptides and therapeutic compositions comprising such trifunctional peptides for use in treating diseases related to activated macrophages. In some embodiments, each trifunctional peptide is capable of at least three functions: 1) mediating formation of naturally long half-life lipopeptide/lipoprotein particles upon interaction with lipoproteins, 2) facilitation of the targeted delivery to cells of interest and/or sites of disease, and 3) treatment, prevention, and/or detection of a disease or condition. In certain embodiments, the present invention relates to amphipathic trifunctional peptides consisting of two amino acid domains, wherein upon interaction with plasma lipoproteins, one amino acid domain mediates formation of naturally long half-life lipopeptide/lipoprotein particles and targets these particles to macrophages, whereas the other amino acid domain inhibits the TREM-1/DAP-12 receptor signaling complex expressed on macrophages.

As described herein, surprisingly it was found that potentially therapeutic trifunctional peptides of the present invention are capable of executing at least, three functions (trifunctional peptides): 1) assistance in the self-assembly of naturally long half-life lipopeptide particles upon binding to lipid or lipid mixtures in vitro, i.e. incorporation of the trifunctional peptides as part of the lipid portion of synthetic/recombinant HDLs, then after administration; 2) facilitation of the targeted delivery to cells of interest and/or sites of disease, and 3) treatment, prevention, and/or detection of a disease or condition. Thus in some embodiments, trifunctional peptides, after mixing with lipids in vitro, may assist in the self-assembly of synthetic lipopeptide particles (SLP) upon binding to a lipid or to lipids in mixtures. In the methods of the present invention, the SLP of interest are synthetic nanoparticles that mimic human lipoproteins as recombinant (r)HDLs. While not being bound to any particular theory, it is believed that this interaction and ability to form lipopeptide/lipoprotein particles is mediated by the amphipathic alpha helical sequences of the trifunctional peptides described herein.

Another surprising discovery was that administration of potentially therapeutic trifunctional peptides of the present invention, that were not in rHDL formulations, showed: 1) mediation of formation of naturally long half-life lipopeptide/lipoprotein particles (LP) upon interaction with native lipoproteins in vivo, 2) facilitation of the targeted delivery to cells of interest and/or sites of disease, and 3) treatment, prevention, and/or detection of a disease or condition. Thus in some embodiments, free trifunctional peptides (i.e. not in rHDL formulations) as part of compounds of the present invention, after administration to populations of cells or administration to a mammal, may interact with native lipoproteins and form trifunctional peptide containing lipopeptide/lipoprotein particles in vivo.

Thus, potentially therapeutic trifunctional peptides of the present invention were synthesized and used for targeted treatment and imaging in vivo, as either formulations with HDLs or without, i.e. trifunctional peptides in a pharmaceutical formulation without HDLs.

Advantageous of using the trifunctional peptides described herein in order to solve numerous problems administering therapeutic or diagnostic compounds include avoiding high dosages of other TAs (therapeutic agents) and imaging probes required; and the lack of control and reproducibility of formulations, especially in large-scale production. In other words, using trifunctional peptides described herein, including trifunctional peptide formulations including therapeutic drug compounds, would potentially lower the amount of drug needed to reduce symptoms of a disease.

Another advantage is economic. Therapeutic peptides have relatively high synthetic and production costs, For example, the production cost of a 5000 Da molecular mass peptide exceeds the production cost of a 500 Da molecular mass small molecule, which in turn exceeds the production cost of a 500 Da molecular mass small molecule by more than 10-fold up to less than 100-fold for each increase in magnitude of size. By combining three functions in one peptide significantly simplifies the manufacture of these trifunctional peptides as targeted drugs, and as delivery agents for drug compounds and imaging probes.

### I. Trifunctional Peptides.

The present invention encompasses the discovery that it is possible to combine multiple functions in one polypeptide amino acid sequence, i.e. a trifunctional peptide, in order to confer a variety of properties on the resulting amphipathic multipeptide.

The present disclosure describes novel amphipathic trifunctional peptides and therapeutic compositions comprising such trifunctional peptides for use in treating diseases related to activated immune cells. In some embodiments, each trifunctional peptide is capable of at least three functions: 1) mediating formation of naturally long half-life lipopeptide/lipoprotein particles upon interaction with lipoproteins, 2) facilitation of the targeted delivery to cells of interest and/or sites of disease, and 3) treatment, prevention, and/or detection of a disease or condition. In some embodiments, each trifunctional peptide is capable of at least three functions: 1) mediating the self-assembly of naturally long half-life lipopeptide particles upon binding to lipid or lipid mixtures, 2) facilitation of the targeted delivery to cells of interest and/or sites of disease, and 3) treatment, prevention, and/or detection of a disease or condition. In certain embodiments, the present invention relates to amphipathic trifunctional peptides consisting of two amino acid domains, wherein upon interaction with plasma lipoproteins, one amino acid domain mediates formation of naturally long half-life lipopeptide/lipoprotein particles and targets these particles to macrophages, whereas the other amino acid domain inhibits the TREM-1/DAP-12 receptor signaling complex expressed on macrophages. The invention further relates to personalized medical treatments for cancer that involve targeting specific cancers by their tumor environment.

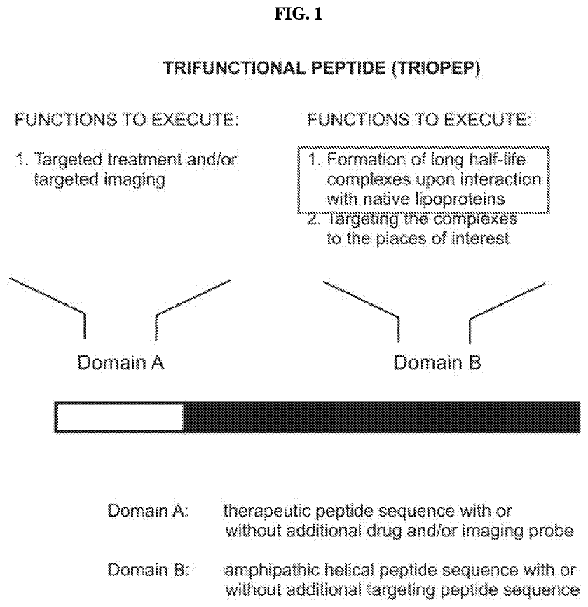

In preferred embodiments, trifunctional peptides of the present invention comprise two amino acid domains (See FIG. 1): domain A that confers therapeutic and/or diagnostic benefits in the context of the treatment, prevention, and/or detection of a disease or condition; and domain B that confers multiple benefits in the context of: 1A) formation of long half-life lipopeptide particles upon binding to lipid or lipid mixtures in vitro 1B) formation of long half-life LP upon interaction with lipoproteins in vivo, and 2) the targeted delivery of the particles formed to cells of interest and/or sites of disease or condition.

In one embodiment, the present invention includes a resulting trifunctional peptide comprising: (a) one amino acid domain that confers therapeutic and/or diagnostic benefits in the context of the treatment, prevention, and/or detection of a disease or condition; and (b) another amino acid domain that confers multiple benefits in the context of the self-assembly of naturally long half-life SLP and LP upon binding to lipid or lipid mixtures and targeted delivery of the particles formed to cells of interest and/or sites of disease or condition. In one embodiment, any or both the domains comprise minimal biologically active amino acid sequence. In one embodiment, the first amino acid domain comprises a cyclic peptide sequence. In one embodiment, the first amino acid domain comprises a disulfide-linked dimer. In one embodiment, any or both of the amino acid domains include amino acids selected from the group of natural and unnatural amino acids including, but not limited to, L-amino acids, or D-amino acids.

In one embodiment, one or both amino acid domains of the peptides and compounds of the present invention are conjugated to a drug compound (therapeutic agent: TA). In one embodiment, a therapeutic agent is selected from the group including, but not limited to, anticancer, antibacterial, antiviral, autoimmune, anti-inflammatory and cardiovascular agents, antioxidants, and therapeutic peptides. In one embodiment, the therapeutic agent is a hydrophobic therapeutic agent. The therapeutic agent may also be selected from the group comprising paclitaxel, valrubicin, doxorubicin, taxotere, campotechin, etoposide, and any combination thereof.

In one embodiment, one or both amino acid domains of the peptides and compounds of the present invention are conjugated to an imaging probe. In one embodiment, the imaging agent is a Gd-based contrast agent (GBCA) for magnetic resonance imaging (MRI). In one embodiment, the imaging agent is a [⁶⁴Cu]-containing imaging probe for imaging systems such as a positron emission tomography (PET) imaging systems (and combined PET/computer tomography (CT) and PET/MRI systems). In one embodiment, an imaging probe and/or an additional therapeutic agent is conjugated to any or both of the domains. In one embodiment, the peptides and compounds of the present invention are used in combinations thereof.

Although many examples describe or show results of using trifunctional peptides in formulations with rHDLs, it is not meant to limit the use of these trifunctional peptide sequences in HDL formulations. Conversely, examples describing or showing results of using trifunctional peptides alone, or in formulations without rHDLs is not meant to limit the use of such trifunctional peptides without rHDLs. Thus, in certain embodiments, the trifunctional peptides of the present invention may be administered within rHDLs, or administered in pharmaceutical formulations as part of rHDLs. In other embodiments, the trifunctional peptides of the present invention may be administered without rHDLs, or administered in pharmaceutical formulations without rHDLs.

In one embodiment, the peptides of the present invention form lipopeptide particles in vitro. In one embodiment, the peptides of the present invention form lipopeptide particles in vivo. In certain embodiments, the present invention relates to peptides consisting of two amino acid domains, wherein upon binding to lipid or lipid mixtures, one amino acid domain assists in the self-assembly of naturally long half-life lipopeptide particles and targets these particles to macrophages, whereas another amino acid domain inhibits TREM-1/DAP-12 receptor complex expressed on macrophages.

In certain embodiments, the present invention relates to peptides comprising at least two amino acid domains, wherein upon binding to lipid or lipid mixtures, the first amino acid domain assists in the self-assembly of naturally long half-life lipopeptide particles and targets these particles to macrophages, whereas the second amino acid domain inhibits TREM-1/DAP-12 receptor complex expressed on macrophages.

In certain embodiments, the peptides of the present invention self-assemble upon binding to lipid or lipid mixtures in vitro to form synthetic lipopeptide particles (SLP) that mimic human lipoproteins and have a long half-life in a bloodstream. In one embodiment, the peptides and compounds of the present invention interact with endogenous lipoproteins in vivo and form long half-life LP. In one embodiment, the peptides and compounds of the present invention are used in combinations thereof.

The peptides and compounds of the present invention and combinations thereof alone as well as the SLP formed upon their binding to lipid or lipid mixtures have a wide variety of uses, particularly in the areas of oncology, transplantology, dermatology, hepatology, ophthalmology, cardiovascular diseases, sepsis, autoimmune diseases, neurodegenerative diseases and other diseases and conditions. They also are useful in the production of medical devices (for example, medical implants and implantable devices).

The invention disclosed herein provides for methods of treating cancer using inhibitors of the TREM-1 pathway. These inhibitors include peptide variants and compositions that modulate the TREM-1-mediated immunological responses beneficial for the treatment of cancer. The invention also provides for predicting the efficacy of TREM-1-targeted therapies in various cancers by analyzing biological samples for the presence of myeloid cells and for the TREM-1 expression levels. In one embodiment, the present invention relates to the targeted treatment, prevention and/or detection of cancer including but not limited to pancreatic cancer, breast cancer, liver cancer, multiple myeloma, leukemia, bladder cancer, CNS cancer, stomach cancer, prostate, colorectal cancer, brain cancer, ovarian cancer, renal cancer, skin cancer, osteosarcoma and other cancers and cancer cachexia.

The invention disclosed herein provides for methods of treating cancer using inhibitors of the TREM-1 pathway. These inhibitors include peptide variants and compositions that modulate the TREM-1-mediated immunological responses beneficial for the treatment of cancer. The invention also provides for predicting the efficacy of TREM-1-targeted therapies in various cancers by analyzing biological samples for the presence of myeloid cells and for the TREM-1 expression levels. In one embodiment, the present invention relates to the targeted treatment, prevention and/or detection of cancer including but not limited to pancreatic cancer, breast cancer, liver cancer, multiple myeloma, leukemia, bladder cancer, CNS cancer, stomach cancer, prostate, colorectal cancer, brain cancer, ovarian cancer, renal cancer, skin cancer, osteosarcoma and other cancers and cancer cachexia.

The invention disclosed herein provides for methods of treating cancer using modulators of the TREM-1/DAP-12 signaling pathway. These compounds and compositions modulate the TREM-1-mediated immunological responses beneficial for the treatment of cancer in standalone and combination-therapy treatment regimen. The invention also provides for predicting the efficacy of TREM-1 modulatory therapies in patients with various cancers. In one embodiment, the present invention relates to the targeted treatment, prevention and/or detection of cancer including but not limited to lung cancer including non-small cell lung cancer, pancreatic cancer, breast cancer, liver cancer, multiple myeloma, melanoma, leukemia, bladder cancer, central nervous system cancer, stomach cancer, prostate cancer, colorectal cancer, colon cancer, brain cancer, gastrointestinal cancer, gastric cancer, ovarian cancer, renal cancer, skin cancer, osteosarcoma, endometrial cancer, esophageal cancer, kidney cancer, thyroid cancer, neuroblastoma, neurofibroma, glioma, glioblastoma, glioblastoma multiforme, head and neck cancer, cervical cancer, giant cell tumor of the tendon sheath, tenosynovial giant cell tumor, pigmented villonodular synovitis, and other cancers in which myeloid cells are involved or recruited and cancer cachexia.

The invention disclosed herein provides for methods of treating cancer using inhibitors of the TREM-1 pathway. These inhibitors include peptide variants and compositions that modulate the TREM-1-mediated immunological responses beneficial for the treatment of cancer. The invention also provides for predicting the efficacy of TREM-1-targeted therapies in various cancers by analyzing biological samples for the presence of myeloid cells and for the TREM-1 expression levels. In one embodiment, the present invention relates to the targeted treatment, prevention and/or detection of cancer including but not limited to pancreatic cancer, breast cancer, liver cancer, multiple myeloma, leukemia, bladder cancer, CNS cancer, stomach cancer, prostate, colorectal cancer, brain cancer, ovarian cancer, renal cancer, skin cancer, osteosarcoma and other cancers and cancer cachexia.

The invention disclosed herein provides for methods of treating scleroderma using modulators of the TREM-1/DAP-12 signaling pathway. These compounds and compositions modulate the TREM-1-mediated immunological responses beneficial for the treatment of scleroderma or a related autoimmune or a fibrotic condition in standalone and combination-therapy treatment regimen. The invention also provides for predicting the efficacy of TREM-1 modulatory therapies in patients with scleroderma. In one embodiment, the present invention relates to the targeted treatment, prevention and/or detection of scleroderma including but not limited to calcinosis, Raynaud's phenomenon, esophageal dysmotility, scleroderma, or telangiectasia syndrome (CREST).

In one embodiment, the present invention relates to the targeted treatment, prevention and/or detection of cancer including but not limited to lung, pancreatic, breast, stomach, prostate, colon, brain and skin cancers, cancer cachexia, atherosclerosis, allergic diseases, acute radiation syndrome, inflammatory bowel disease, empyema, acute mesenteric ischemia, hemorrhagic shock, multiple sclerosis, autoimmune diseases, including but not limited to, atopic dermatitis, lupus, scleroderma, rheumatoid arthritis and other rheumatic diseases, sepsis and other inflammatory diseases or other condition involving myeloid cell activation and, more particularly, TREM receptor-mediated cell activation, including but not limited to diabetic retinopathy and retinopathy of prematurity, Alzheimer's, Parkinson's and Huntington's diseases.

The disclosure also provides for a method of treating, preventing and/or detecting an immune-related condition. The method comprises providing a composition comprising peptides and compounds of the present disclosure and/or a synthetic nanoparticle formed upon their binding to lipid or lipid mixtures, a patient having at least one symptom of a disease or condition in which the immune system is involved, and administering the composition to the patient under conditions such that said one symptom is reduced. The immune-related condition of the method may include a heart disease, atherosclerosis, peripheral artery disease, restenosis, stroke, multiple sclerosis, the cancers (e.g., sarcoma, lymphoma, leukemia, carcinoma and melanoma), bacterial infectious diseases, acquired immune deficiency syndrome (AIDS), allergic diseases, autoimmune diseases (e.g., atopic dermatitis, psoriasis, rheumatoid arthritis, Sjogren's syndrome, scleroderma, systemic lupus erythematosus, non-specific vasculitis, Kawasaki's disease, psoriasis, type I diabetes, pemphigus vulgaris), granulomatous diseases (e.g., tuberculosis, sarcoidosis, lymphomatoid granulomatosis, Wegener's granulomatosus), Gaucher's disease, inflammatory diseases (e.g., sepsis, inflammatory lung diseases such as chronic obstructive pulmonary disease (COPD), interstitial pneumonitis and asthma, retinopathy such as diabetic retinopathy and retinopathy of prematurity, inflammatory bowel disease such as Crohn's disease, and inflammatory arthritis), liver diseases (e.g., alcoholic liver disease and nonalcoholic fatty liver disease), neurodegenerative diseases such as Alzheimer's, Parkinson's and Huntington's diseases, and transplant (e.g., heart/lung transplants) rejection reactions.

The invention relates to personalized medical treatments for scleroderma. More specifically, the invention provides for treatment of scleroderma or a related autoimmune or a fibrotic condition by using inhibitors of the TREM-1/DAP-12 pathway. These inhibitors include peptide variants and compositions that modulate the TREM-1-mediated immunological responses beneficial for the treatment of scleroderma. In addition, the invention provides for predicting the efficacy of TREM-1-targeted therapies in scleroderma by analyzing biological samples for the presence of myeloid cells and for the TREM-1 expression levels. In one embodiment, the peptides and compositions of the present invention modulate TREM-1/DAP-12 receptor complex expressed on macrophages. In one embodiment, the peptides and compositions of the invention are conjugated to an imaging probe. In one embodiment, the invention provides for detecting the TREM-1-expressing cells and tissues in an individual with scleroderma using imaging techniques and the peptides and compositions of the invention containing an imaging probe. In one embodiment, the peptides and compositions of the invention are used in combinations thereof. In one embodiment, the peptides and compositions of the invention are used in combinations with other antifibrotic therapeutic agents. In one embodiment, the present invention relates to the targeted treatment, prevention and/or detection of scleroderma including but not limited to calcinosis, Raynaud's phenomenon, esophageal dysmotility, scleroderma, or telangiectasia syndrome (CREST).

II. Trifunctional Peptides in rHDL (SLP) Formulations.

In one embodiment, the SLP self-assembled upon binding of the peptides and compounds of the present invention and combinations thereof to lipid or lipid mixtures are discoidal or spherical in shape. While the size of the particles is preferably between 5 nm and 50 nm, the diameter may be up to 200 nm. In one embodiment, the lipid of the particles may include cholesterol, a cholesteryl ester, a phospholipid, a glycolipid, a sphingolipid, a cationic lipid, a diacylglycerol, or a triacylglycerol. And further, the phospholipid may include phosphatidylcholine (PC), phosphatidylethanolamine (PE), phosphatidylserine (PS), phosphatidylinositol (PI), phosphatidylglycerol (PG), cardiolipin (CL), sphingomyelin (SM), or phosphatidic acid (PA). And even further, the cationic lipid can be 1,2-dioleoyl-3-trimethylammonium-propane (DOTAP). The lipid of the synthetic nanoparticle may be polyethylene glycol(PEG)ylated. In certain embodiments, the peptides and compounds of the present invention and/or the SLP and LP formed by these peptides and compounds may pass the blood-brain barrier (BBB). In one embodiment, the peptides and compounds of the present invention and/or the SLP and LP formed by these peptides and compounds may pass the blood-retinal barrier (BRB). In one embodiment, the peptides and compounds of the present invention and/or the SLP and LP formed by these peptides and compounds may pass the blood-tumor barrier (BTB).

In certain embodiments, the peptides and compounds of the present invention include an amino acid sequence derived from apo A-I, A-II, A-IV, B, C-I, C-II, C-III, or E. In one embodiment, the peptides and compounds of the present invention include an amino acid sequence derived from apo A-I, A-II, A-IV, B, C-I, C-II, C-III, or E and Arginine-glycine-aspartic acid (RGD)-peptide sequence. In certain embodiments, the peptides and compounds of the present invention include an amino acid sequence derived from transmembrane domain sequences of human or animal cell-surface receptors and of signaling subunits thereof. In certain embodiments, the peptides and compounds of the present invention include an amino acid sequence derived from virus membrane fusion and structural proteins. In one embodiment, the peptides and compounds of the present invention include an amino acid sequence derived from apo A-I, A-II, A-IV, B, C-I, C-II, C-III, or E conjugated to a targeting moiety to enhance the targeting efficacy of the therapeutic agent. The targeting moiety may include a polypeptide, an antibody, a receptor, a ligand, a peptidomimetic agent, an aptamer or a product of phage display.

In one embodiment, the amino acid domains of the peptides and compounds of the present invention comprise unmodified or modified peptide sequences. The modified peptide sequence may contain at least one amino acid residue which is chemically or enzymatically modified. The modified amino acid residue may be an oxidized amino acid residue. The oxidized amino acid residue may be a methionine residue. The modified peptide sequence may contain at least one amino acid residue, which is oxidized, halogenated, or nitrated. The modified peptide sequence may include an amphipathic amino acid sequence.

In certain embodiments, the present invention relates to the targeted treatment or prevention of inflammatory or other condition involving myeloid cell activation and, more particularly, TREM receptor-mediated cell activation, such as cancer including but not limited to, lung, pancreatic, breast, stomach, prostate, colon, brain and skin cancers, cancer cachexia, atherosclerosis, allergic diseases, acute radiation syndrome, inflammatory bowel disease, empyema, alcohol-induced liver disease, nonalcoholic fatty liver disease and non-alcoholic steatohepatitis, acute mesenteric ischemia, hemorrhagic shock, multiple sclerosis, sepsis, diabetic retinopathy and retinopathy of prematurity, Alzheimer's, Parkinson's and Huntington's diseases, autoimmune diseases, including but not limited to, atopic dermatitis, lupus, scleroderma, rheumatoid arthritis and other rheumatic diseases.

In one embodiment, the present invention provides a pharmaceutical composition comprising the peptides and compounds and combinations thereof alone or the SLP nanoparticles self-assembled upon binding of these peptides and compounds to lipid or lipid mixtures.

A. TREM-1-Related Trifunctional Peptides.

TREM-1 is expressed on the majority of innate immune cells and to a lesser extent on parenchymal cells. Upon activation, TREM-1 can directly amplify an inflammatory response. Although it was initially demonstrated that TREM-1 was predominantly associated with infectious diseases, recent evidences demonstrate that TREM-1 receptor and its signaling pathways contribute to the pathology of non-infectious acute and chronic inflammatory diseases, including but not limiting to, rheumatoid arthritis, atherosclerosis, ischemia reperfusion-induced tissue injury, colitis, fibrosis, neurodegenerative diseases, liver diseases, retinopathies, and cancer (see e.g., Tammaro, et al. Pharmacol Ther 2017, 177:81-95; Saadipour. Neurotox Res 2017, 32:14-16; Sigalov. Adv Protein Chem Struct Biol 2018, 111:61-9; Sigalov. U.S. Pat. No. 8,513,185; Sigalov. U.S. Pat. No. 9,981,004; Rojas, et al. Biochim Biophys Acta 2018, 1864: 2761-2768, Tornai, et al. Hepatology Communications 2018, in press, and Kuai, et al. US 2008/0247955).

In certain embodiments, a resulting trifunctional peptide of the present invention comprises two amino acid domains, wherein one domain comprises a variant TREM-1 inhibitory amino acid sequence and functions to inhibit TREM-1/DAP-12 receptor complex expressed on myeloid cells (e.g. macrophages), whereas another amino acid domain comprises the chemically and/or enzymatically modified amino acid sequence derived from apolipoprotein amino acid sequences and functions to assist in the self-assembly of SLP upon binding to lipid or lipid mixtures in vitro and/or to form LP in vivo, respectively, and to target these particles to myeloid cells (e.g. macrophages). In one embodiment, the TREM-1 inhibitory amino acid domain is the N-terminal domain of a resulting peptide. In one embodiment, the TREM-1 inhibitory amino acid domain is the C-terminal domain of a resulting peptide. In one embodiment, the TREM-1 inhibitory amino acid domain comprises a cyclic peptide sequence. In one embodiment, the TREM-1 inhibitory amino acid domain comprises a disulfide-linked dimer. In one embodiment, the TREM-1 inhibitory amino acid domain includes the group of natural and unnatural amino acids including, but not limited to, L-amino acids, or D-amino acids. In one embodiment, an imaging agent is conjugated to the TREM-1 inhibitory amino acid domain or to the apolipoprotein amino acid sequence-derived domain or to both.

In some preferred embodiments, TREM-1-related peptides and associated compositions of the present invention have a domain A conjugated to a domain B. See, FIG. 1. Domain A comprises a TREM-1 modulatory peptide sequence designed using a known model of cell receptor signaling, the Signaling Chain HOmoOLigomerization model, capable of modulating TREM-1 receptor expressed on myeloid cells (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. U.S. Pat. No. 8,513,185; and Sigalov. U.S. Pat. No. 9,981,004), all of which are herein incorporated by reference in their entirety.

In some preferred embodiments, peptides and compositions of this class further comprise the domain B comprising at least one modified or unmodified amphipathic alpha helical peptide fragment, such as a apo A-I and/or A-II peptide fragment, to form upon interaction with lipid and/or lipid mixtures. In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

FIG. 1 presents an exemplary schematic representation of one embodiment of a trifunctional peptide of the present invention comprising amino acid domains A and B where amino acid domain A represents a therapeutic peptide sequence with or without an attached drug compound and/or imaging probe that functions to treat, prevent and/or detect a disease or condition, whereas amino acid domain B represents an amphipathic alpha helical peptide sequence, with or without an additional targeting peptide sequence, and functions to 1) assist in the self-assembly of synthetic lipoprotein/lipopeptide nanoparticles (SLP) upon interaction with lipids or lipid mixtures in vitro, for use in transporting these trifunctional peptides as lipoprotien nanoparticles to sites of interest in vitro or in vivo and/or 2) form long half-life lipopeptide/lipoprotein particles upon interaction with endogenous lipoproteins for transporting these trifunctional peptides to the sites of interest. Endogenous lipoproteins may be lipoproteins added to or found in cell cultures, or lipoprotiens in a mammalian body.

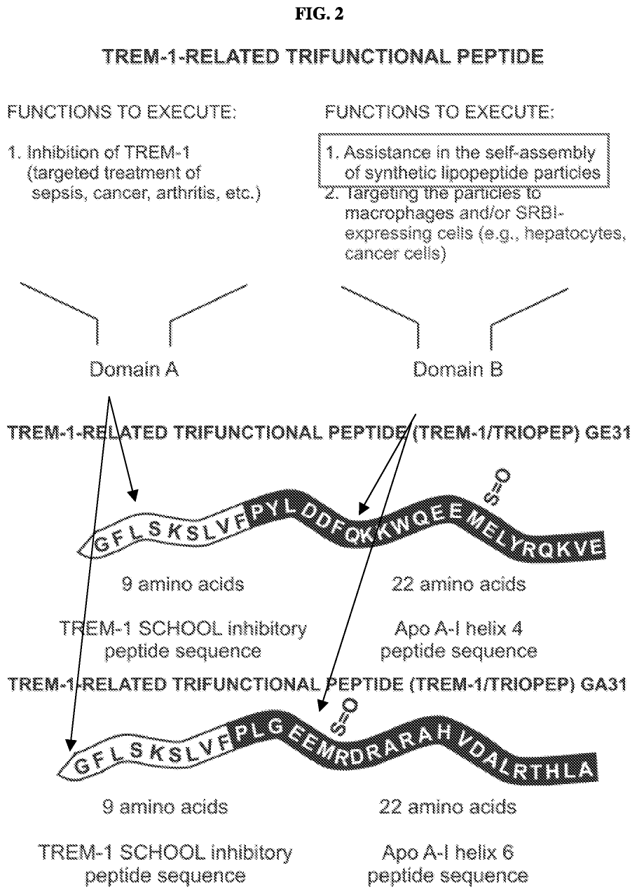

In certain embodiments, FIG. 2 shows the structures of representative TREM-1-related trifunctional peptides, TREM-1/TRIOPEP GE31 (GFLSKSLVFPYLDDFQKKWQEEM(O)ELYRQKVE, M(O), methionine sulfoxide) (SEQ ID NO. 4) and TREM-1/TRIOPEP GA31 (GFLSKSLVFPLGEEM(O)RDRARAHVDALRTHLA, M(O), methionine sulfoxide) (SEQ ID NO. 3). In one embodiment, methionine residues of the peptides TREM-1/TRIOPEP GE31 (GFLSKSLVFPYLDDFQKKWQEEMELYRQKVE) (SEQ ID NO. 2) and TREM-1/TRIOPEP GA31 (GFLSKSLVFPLGEEMRDRARAHVDALRTHLA) (SEQ ID NO. 1) are unmodified. GFLSKSLVF, is found within isoform 1 of human TREM-1 (UniProtKB—Q9NP99 (TREM1_HUMAN), and in human TREM-1 isoform CRA_a (UniProtKB—Q38L15 (Q38L15_HUMAN), both downloaded Oct. 24, 2018)). Peptide GFLSKSLVF is also described without an attached apo I peptide domain, in, for examples, WO 2011/047097 “Inhibition of trem receptor signaling with peptide variants.” Publication Date: 21 Apr. 2011, U.S. Pat. No. 9,981,004B2 “Inhibition of TREM receptor signaling with peptide variants.” Published Jun. 5, 2014, each of which is herein incorporated by reference in its entirety. Sequence information was downloaded Oct. 25, Oct. 26 or Oct. 27, 2019.

Q9NP99|TREM1_HUMAN Isoform 1 Triggering receptor expressed on myeloid cells 1, Homo sapiens:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKSTADVSTPDSEINLTNVTDIIR
VPVFNIVILLAGGFLSKSLVFSVLFAVTLRSFVP

Q38L15|Q38L15_HUMAN Triggering receptor expressed on myeloid cells 1, Homo sapiens isoform CRA_a:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKSTADVSTPDSEINLTNVTDIIR
VPVFNIVILLAGGFLSKSLVFSVLFAVTLRSFVP

GFLSKSLVF, is not found within human TREM-1 isoforms 2 or 3.
Q9NP99-2|TREM1_HUMAN Isoform 2 of Triggering receptor expressed on myeloid cells 1, Homo sapiens:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFRCSTLSFSWLVDS

Q9NP99-3|TREM1_HUMAN Isoform 3 of Triggering receptor expressed on myeloid cells 1, Homo sapiens:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKST.

FIG. 2 presents schematic representations of embodiments of a TREM-1-related trifunctional peptide (TREM-1/TRIOPEP). GE31 (GFLSKSLVFPYLDDFQKKWQEEM(O)ELYRQKVE, M(O), methionine sulfoxide) comprises amino acid domain A and B (GFLSKSLVFPYLDDFQKKWQEEM(O)ELYRQKVE) where domain A represents a 9 amino acids-long human TREM-1 inhibitory therapeutic SCHOOL peptide sequence and functions to treat and/or prevent a TREM-1-related disease or condition, whereas domain B represents a 22 amino acids-long human apolipoprotein A-I helix 4 peptide sequence with a sulfoxidized methionine residue and functions to assist in the self-assembly of synthetic lipopeptide particles (SLP) in vitro for targeting the particles to myeloid cells (e.g. macrophages). GA31 (GFLSKSLVFPLGEEM(O)RDRARAHVDALRTHLA, M(O), methionine sulfoxide).
Abbreviations: apo, apolipoprotein; SCHOOL, signaling chain homooligomerization; TREM-1, triggering receptor expressed on myeloid cells-1.

Imaging of TREM-1 Expression.

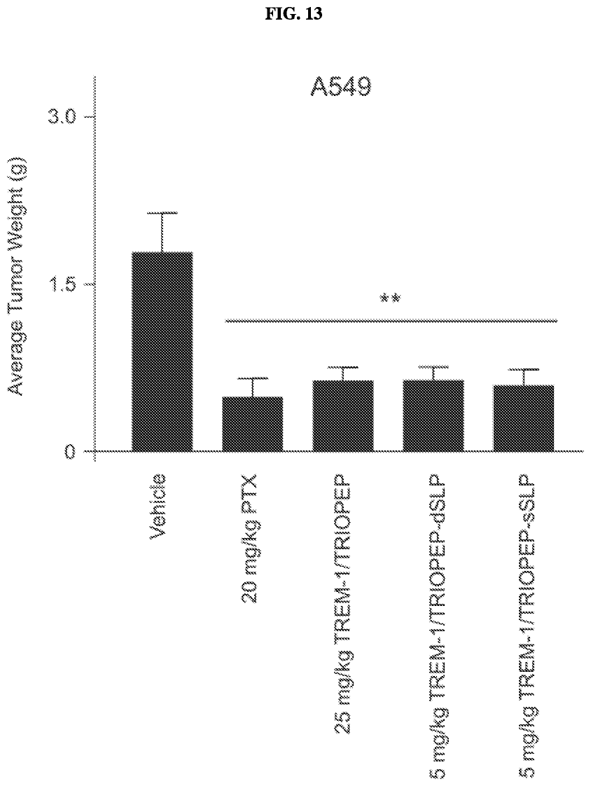

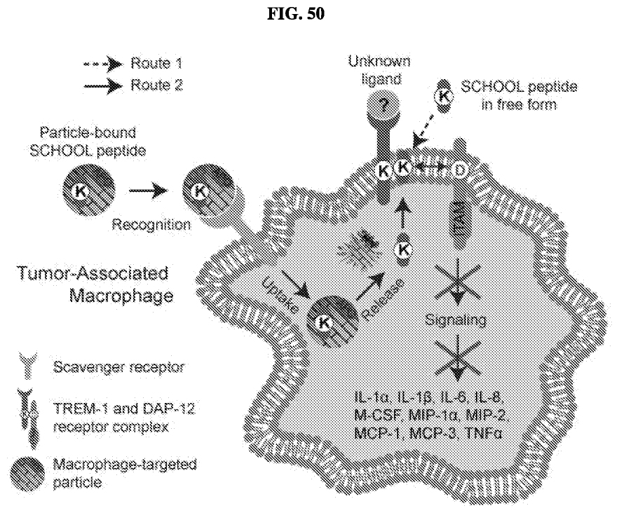

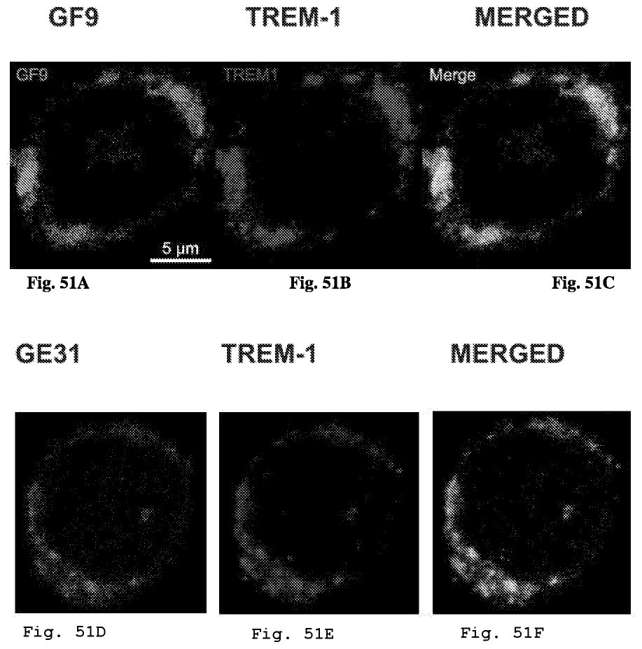

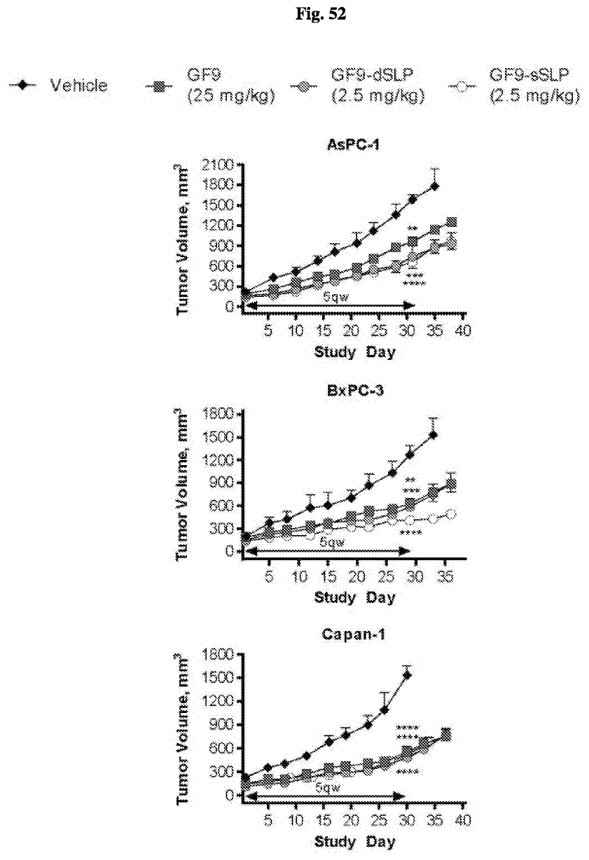

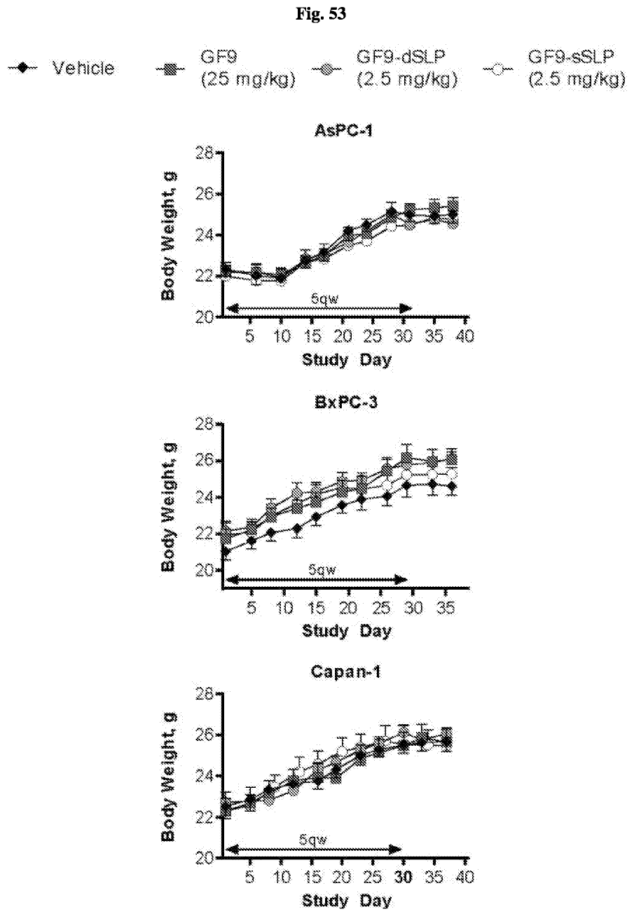

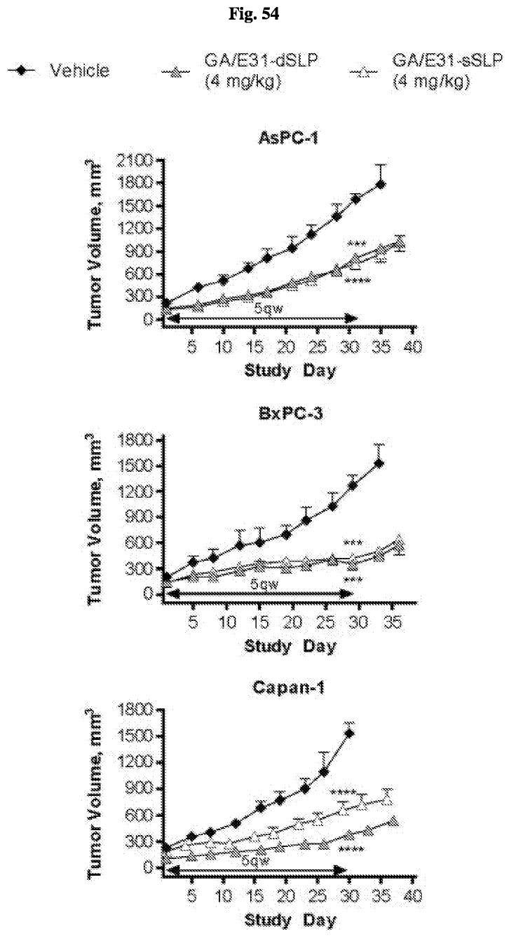

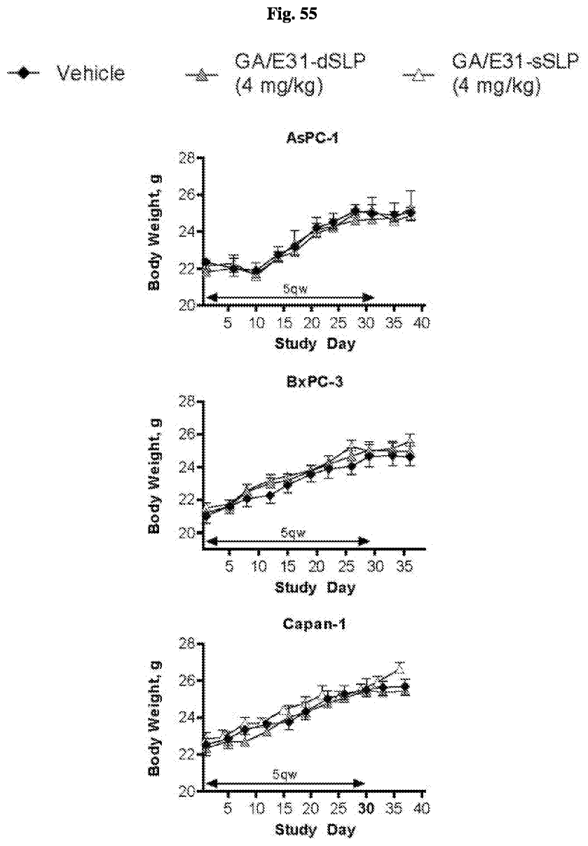

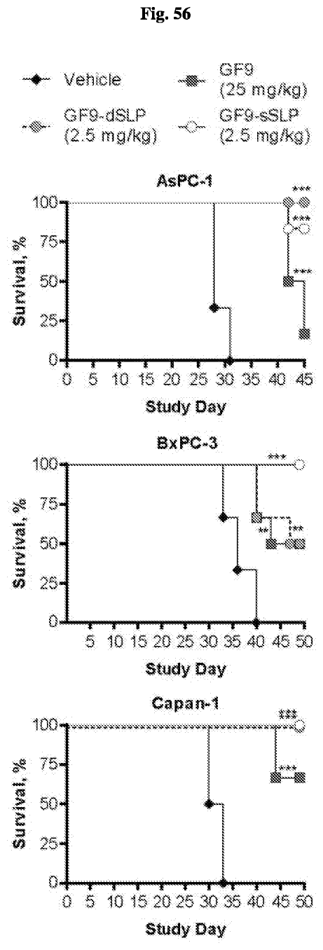

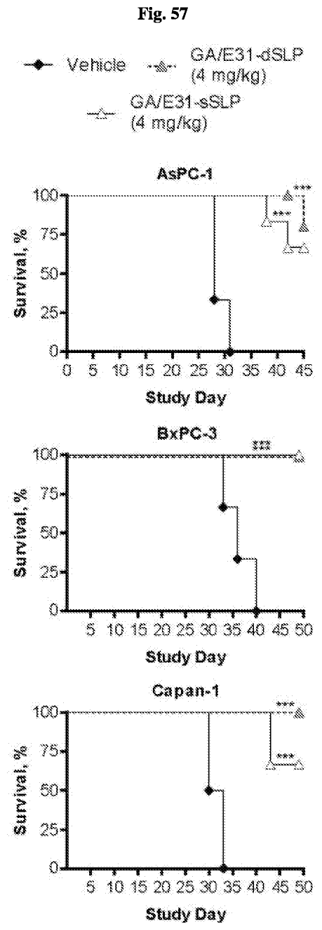

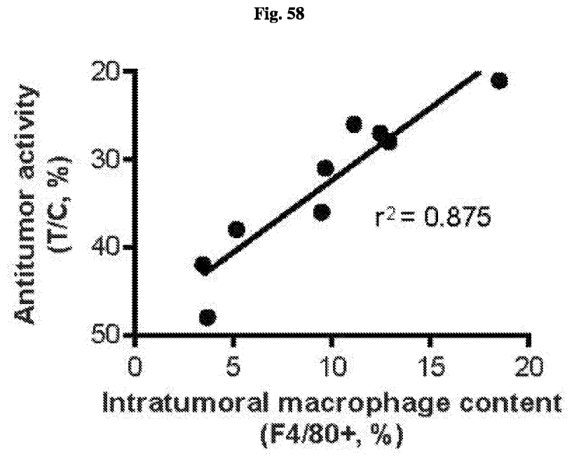

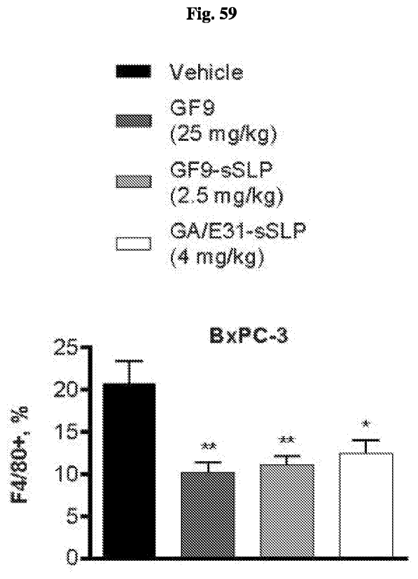

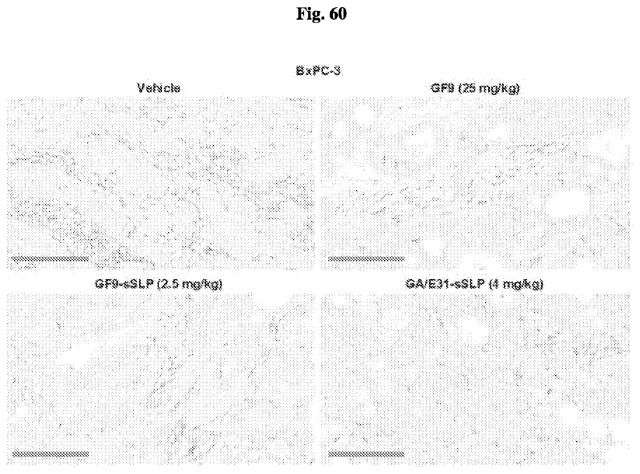

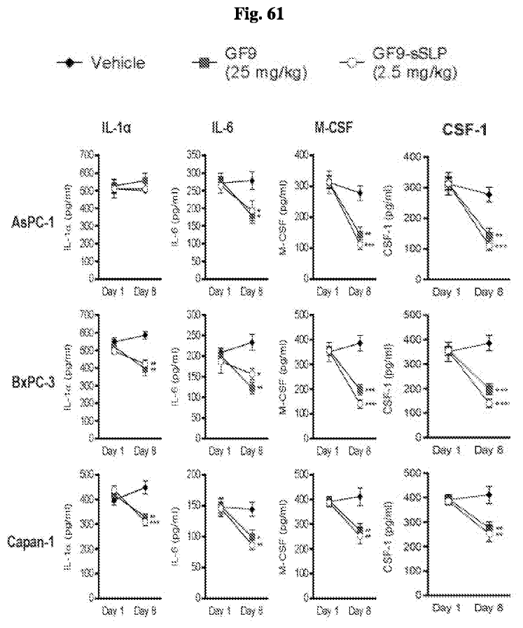

Another way to evaluate the TREM-1 expression level is to use imaging (visualization) techniques and procedures. In one embodiment, FIG. 50 shows that the fluorescently labeled TREM-1/TRIOPEP peptide GE31 delivered to macrophages by the SLP particles colocalizes with TREM-1 expressed on these cells. See also (Rojas et al. 2018). As described herein and in (Rojas et al. 2018), TREM-1 inhibitory therapy using the modulators of the TREM-1/DAP-12 signaling pathway results in reduction of tissue TREM-1 expression as measured by Western Blot (See FIG. 13).

In certain embodiments, the capability of the modulators of the TREM-1/DAP-12 signaling pathway described herein, including but not limited to, anti-TREM-1 blocking antibodies and fragments thereof, TREM-1 inhibitory SCHOOL peptides (e.g., GF9) and trifunctional TREM-1 inhibitory peptides including but not limited to, GA31 and GE31, to colocalize with TREM-1 can be used to visualize (image) this receptor and evaluate its expression/level in the areas of interest. In one embodiment, for this purpose, an imaging probe (e.g. [⁶⁴Cu], see TABLE 3) can be conjugated to the peptide sequences, GE31 (GFLSKSLVFPYLDDFQKKWQEEM(O)ELYRQKVE, M(O), methionine sulfoxide) (SEQ ID NO. 27) and/or GA31 (GFLSKSLVFPLGEEM(O)RDRARAHVDALRTHLA (M(O), methionine sulfoxide) (SEQ ID NO. 26). In one embodiment, methionine residues of the peptides GE31 (GFLSKSLVFPYLDDFQKKWQEEMELYRQKVE) (SEQ ID NO. 25) and GA31 (GFLSKSLVFPLGEEMRDRARAHVDALRTHLA) (SEQ ID NO. 24) are unmodified. In one embodiment, imaging (visualization) of TREM-1 levels using the labeled modulators described herein and the PET and/or other imaging techniques can be used to diagnose GBM and/or to select and monitor novel GBM therapies as disclosed in WO 2017083682A1 and described in (Johnson et al. 2017, Liu et al. 2019). In certain embodiments, imaging (visualization) of TREM-1 levels can be used to diagnose other TREM-1-related diseases and conditions as well as to monitor novel therapies for these diseases and conditions.

GF9 immunotherapy targets pathways restricted to pathological conditions and is highly competitive. In some embodiments, safe and effective GF9 therapies are contemplated for use on pancreatic cancer (PC) to be used in combination with standard first-line treatments: FOLFIRINOX (5-FU, leucovorin, irinotecan and oxaliplatin) or Gemzar®+ABRAXANE®. In some embodiments, advantages for using free GF9 peptide for treating PVNS include but are not limited to: Low toxicity; Proven efficacy in vivo, including joints; easy formulation development; easy scale-up process; Easy and fast GMP production; Low cost of production; and Stable and easy to store.

| Therapy* | | | | |
|---|---|---|---|---|
| * Shown for Cancer | Acute | | | |
| Indications | toxicity | Risk of side effects | Administration | Cost |
| GF9 | LOW | LOW | Systemic/ | LOW |
| immunotherapy | | | Intranasal/ | |
| (as described herein) | | | Pulmonary/ | |
| | | | Oral | |
| Cytotoxic drugs | HIGH | HIGH | Systemic/Oral | HIGH |
| (Gemzar, Abraxane, | | | | |
| Temozolomide) | | | | |
| Biologies | LOW | HIGH | Systemic | HIGH |
| (Bevacizumab, | | | | |
| Canakinumab) | | | | |

In certain embodiments, other preferred TREM-1-related trifunctional peptides and compositions of this class comprise the domain A comprising the TREM-1 inhibitory peptide sequences LR12 and LP17 (described in Gibot, et al. Infect Immun 2006, 74:2823-2830; Gibot, et al. Shock 2009, 32:633-637; Gibot, et al. Eur J Immunol 2007, 37:456-466; Joffre, et al. J Am Coll Cardiol 2016, 68:2776-2793; Cuvier, et al. Br J Clin Pharmacol 2018, in press; Zhou, et al. Int Immunopharmacol 2013, 17:155-161; and disclosed in Faure, et al., U.S. Pat. No. 8,013,116; Faure, et al., U.S. Pat. No. 9,273,111; Gibot, et al., U.S. Pat. No. 9,657,081; Gibot and Derive, U.S. Pat. No. 9,815,883; and in Gibot and Derive, U.S. Pat. No. 9,255,136, each of which is herein incorporated by reference in its entirety) while the domain B comprises at least one modified or unmodified amphipathic apo A-I and/or A-II peptide fragment to form upon interaction with lipid and/or lipid mixtures, SLP structures that can be spherical or discoidal. In some embodiments, resulting trifunctional peptide sequences may be radiolabeled and/or contain unmodified or modified methionine residues (TABLE 2) including but not limiting to, the following sequences: LQEEDAGEYGCMPLGEEM(O)RDRARAHVDALRTHLA (M(O), methionine sulfoxide (SEQ ID NO 7), LQEEDAGEYGCMPYLDDFQKKWQEEM(O)ELYRQKVE (M(O), methionine sulfoxide (SEQ ID NO 8), LQVTDSGLYRCVIYHPPPLGEEM(O)RDRARAHVDALRTHLA (M(O), methinone sulfoxide (SEQ ID NO 9), LQVTDSGLYRCVIYHPPPYLDDFQKKWQEEM(O)ELYRQKVE (M(O), methionine sulfoxide (SEQ ID NO 10).

SLP (rHDL) structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Sigalov. US 20130039948; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). The inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

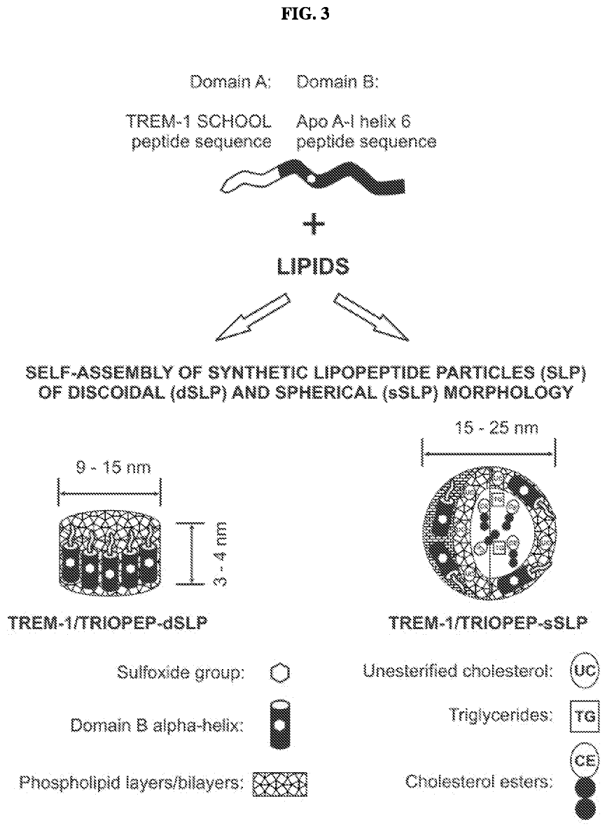

In one embodiment, methionine residues of the peptides TREM-1/TRIOPEP GE31 (GFLSKSLVFPYLDDFQKKWQEEMELYRQKVE) (SEQ ID NO. 2) and TREM-1/TRIOPEP GA31 (GFLSKSLVFPLGEEMRDRARAHVDALRTHLA) (SEQ ID NO. 1) are unmodified. In one embodiment, interaction of TREM-1/TRIOPEP GA31 with lipids results in self-assembly of nanosized SLP of discoidal or spherical morphology (dSLP and sSLP, respectively) (see FIG. 3). FIG. 3 presents a schematic representation of one embodiment of a TREM-1-related trifunctional peptide (TREM-1/TRIOPEP) of the present invention comprising amino acid domains A and B. Depending on lipid mixture compositions added to the peptides, sub 50 nm-sized SLP particles of discoidal (TREM-1/TRIOPEP-dSLP) or spherical (TREM-1/TRIOPEP-sSLP) morphology are self-assembled upon binding of the trifunctional peptide to lipids. Abbreviations: apo, apolipoprotein; SCHOOL, signaling chain homooligomerization; TREM-1, triggering receptor expressed on myeloid cells-1.

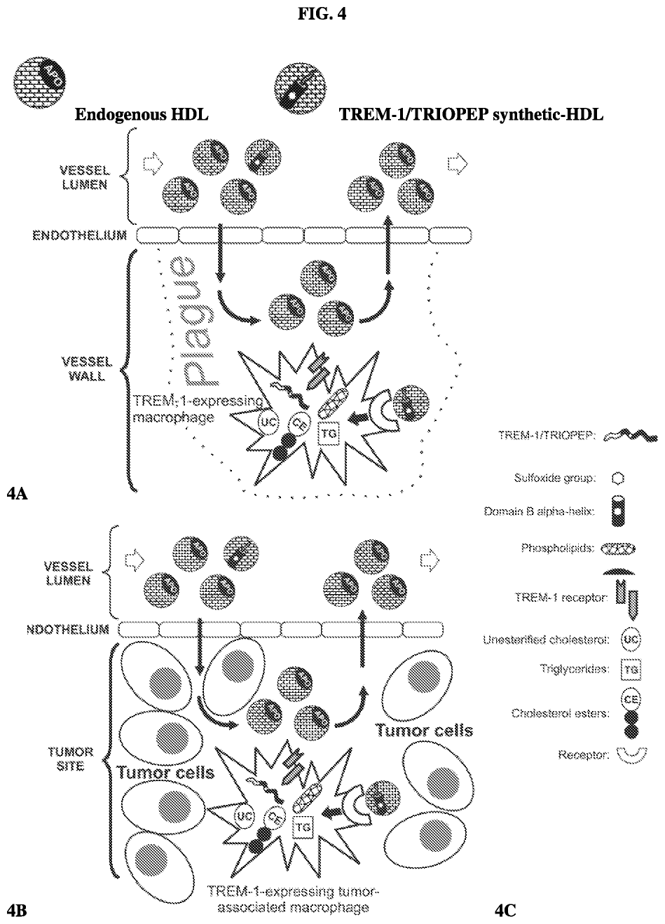

In one embodiment, this provides targeted delivery of the SLP constituents including TREM-1/TRIOPEP to intraplaque macrophages in vivo (FIG. 4A). In one embodiment, this provides targeted delivery of the SLP constituents including TREM-1/TRIOPEP to tumor-associated macrophages (TAMs) in vivo (FIG. 4B).

FIG. 4A illustrates a hypothesized molecular mechanism of action of one embodiment of a trifunctional peptide (TRIOPEP) of the present invention comprising amino acid domains A and B where domain A represents a 9 amino acids-long TREM-1 inhibitory therapeutic peptide sequence and functions to treat and/or prevent a TREM-1-related disease or condition (example, for atherosclerosis), whereas domain B represents a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of atherosclerosis. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in intraplaque macrophages is accompanied by accumulation of TRIOPEP in these cells. In contrast, native HDL particles that contain only unmodified apolipoprotein molecules are not recognized by intraplaque macrophages and return to the circulation.
FIG. 4B illustrates a hypothesized molecular mechanism of action of one embodiment of a trifunctional peptide (TRIOPEP) of the present invention comprising amino acid domains A and B where domain A is a 9 amino acids-long TREM-1 inhibitory therapeutic peptide sequence and functions to treat and/or prevent a TREM-1-related disease or condition (example, for cancer), whereas domain B is a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of cancer. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in tumor-associated macrophages is accompanied by accumulation of TRIOPEP in these cells. In contrast, native HDL particles that contain only unmodified apolipoprotein molecules are not recognized by tumor-associated macrophages and return to the circulation.
FIG. 4C shows a symbol key used in FIGS. 4A-B.

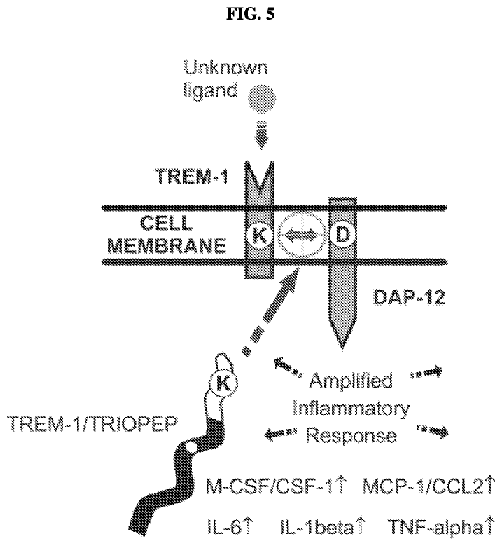

While not being bound to any particular theory, it is believed that in one embodiment, this colocalization is accompanied by a specific disruption of intramembrane interactions between TREM-1 and DAP-12 by the TREM-1-related trifunctional peptide of the present invention (see FIG. 5), resulting in ligand-independent inhibition of TREM-1 upon ligand binding as described in Shen and Sigalov. Mol Pharm 2017, 14:4572-4582 and Shen and Sigalov. J Cell Mol Med 2017, 21:2524-2534, each of which is herein incorporated by reference in its entirety.

FIG. 5 illustrates one embodiment of a specific disruption of intramembrane interactions between TREM-1 and DAP-12 by the trifunctional peptide of the present invention comprising two amino acid domains A and B where domain A is a 9 amino acids-long TREM-1 inhibitory therapeutic peptide sequence, whereas domain B is a 22 amino acids-long apolipoprotein A-I helix 4 or 6 peptide sequence with a sulfoxidized methionine residue. While not being bound to any particular theory, it is believed that this disruption results in “pre-dissociation” of a receptor complex and upon ligand stimulation, leads to inhibition of TREM-1 and silencing the TREM-1 signaling pathway.

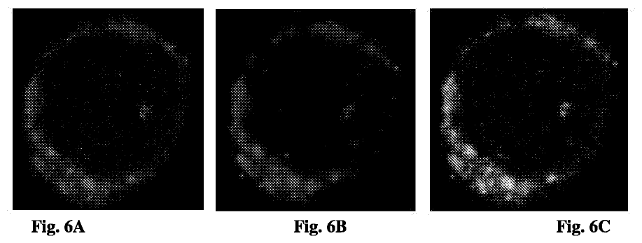

In one embodiment, FIG. 6 shows that the fluorescently labeled TREM-1/TRIOPEP peptide GE31 delivered to macrophages by the SLP particles of the present invention colocalizes with TREM-1 expressed on these cells (see also Rojas, et al. Biochim Biophys Acta 2018, 1864:2761-2768, each of which is herein incorporated by reference in its entirety). In certain embodiments, the capability of the TREM-1-related trifunctional peptides and compounds of the present invention including but not limiting to, TREM-1/TRIOPEP GA31 and TREM-1/TRIOPEP GE31, to colocalize with TREM-1 can be used to visualize (image) this receptor and evaluate its expression in the areas of interest. In one embodiment, for this purpose, an imaging probe (e.g. [⁶⁴Cu], see TABLE 2) can be conjugated to the TREM-1/TRIOPEP sequences, GE31 (GFLSKSLVFPYLDDFQKKWQEEM(O)ELYRQKVE, M(O), methionine sulfoxide) (SEQ ID NO. 4) and GA31 (GFLSKSLVFPLGEEM(O)RDRARAHVDALRTHLA (M(O), methionine sulfoxide) (SEQ ID NO. 3). In one embodiment, imaging (visualization) of TREM-1 levels using PET and/or other imaging techniques can be used to diagnose glioblastoma multiforme (GBM) and/or to select and monitor novel GBM therapies (see e.g., Johnson, et al. Neuro Oncol 2017, 19:vi249 and James and Andreasson, WO 2017083682A1). In certain embodiments, imaging (visualization) of TREM-1 levels can be used to diagnose other TREM-1-related diseases and conditions as well as to monitor novel therapies for these diseases and conditions.

FIG. 6A-C shows images depicting colocalization of the sulfoxidized methionine residue-containing TREM-1-related trifunctional peptide (TREM-1/TRIOPEP) GE31 with TREM-1 in the cell membrane (FIG. 6A), TREM-1 immunohistochemistry staining (FIG. 6B) and a merged image (FIG. 6C).

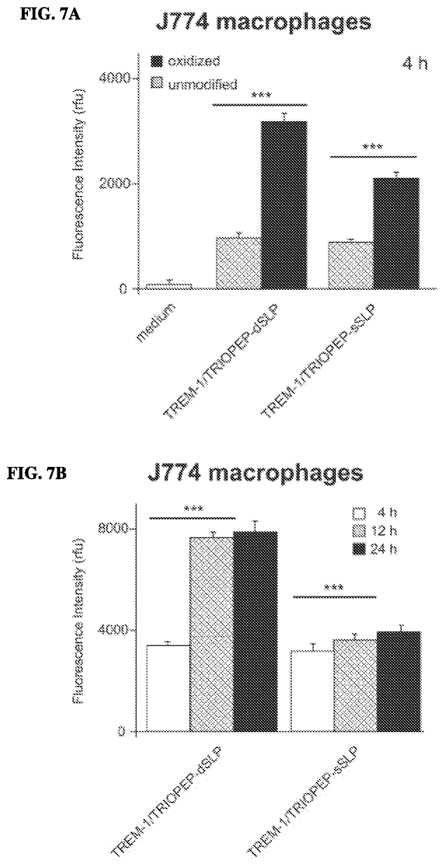

As described herein (see FIG. 7), sulfoxidation of methionine residues in the TREM-1/TRIOPEP peptides GE31 and GA31 results in increased macrophage endocytosis of the SLP containing an equimolar mixture of these peptides (designated as TREM-1/TRIOPEP), TREM-1/TRIOPEP-dSLP and TREM-1/TRIOPEP-sSLP. Shen and Sigalov. Mol Pharm 2017, 14:4572-4582 and Shen and Sigalov. J Cell Mol Med 2017, 21:2524-2534, each of which is herein incorporated by reference in its entirety.

FIG. 7A-B presents the exemplary data showing the endocytosis of synthetic lipopeptide particles (SLP) of discoidal (dSLP) and spherical (sSLP) morphology that contain an equimolar mixture of the TREM-1-related trifunctional peptides (TREM-1/TRIOPEP) GA 31 and GE 31 (TREM-1/TRIOPEP-dSLP and TREM-1/TRIOPEP-sSLP, respectively). (FIG. 7A) The post 4 h incubation in vitro macrophage uptake of TREM-1/TRIOPEP-dSLP and TREM-1/TRIOPEP-sSLP with the unmodified (patterned bars) or sulfoxidized TREM-1/TRIOPEP methionine residues (black bars). ***, P=0.0001 to 0.001 (sulfoxidized vs. unmodified methionine residues). (FIG. 7B) the in vitro macrophage uptake of TREM-1/TRIOPEP-dSLP and TREM-1/TRIOPEP-sSLP with the sulfoxidized TREM-1/TRIOPEP methionine residues post 4 (white bars), 12 (patterned bars), and 24 h (black bars) incubation. ***, P=0.0001 to 0.001 as compared with 4 h incubation time.

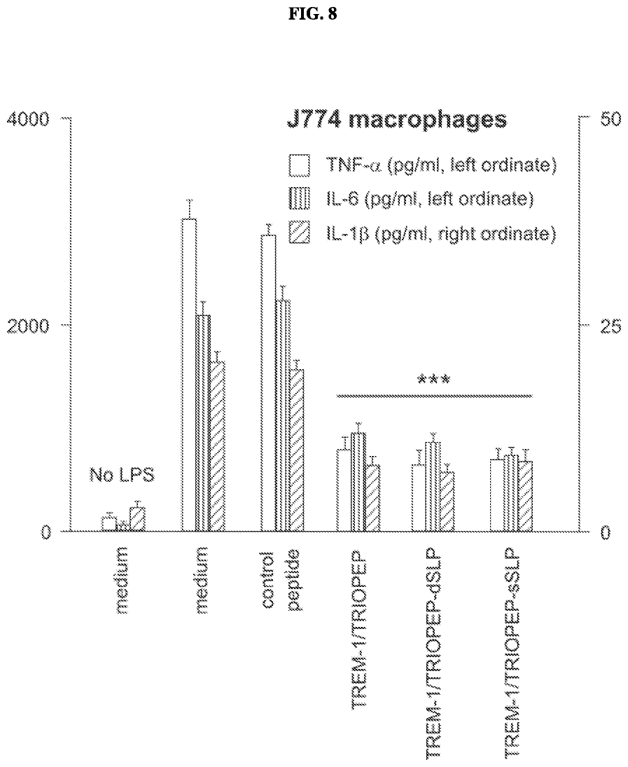

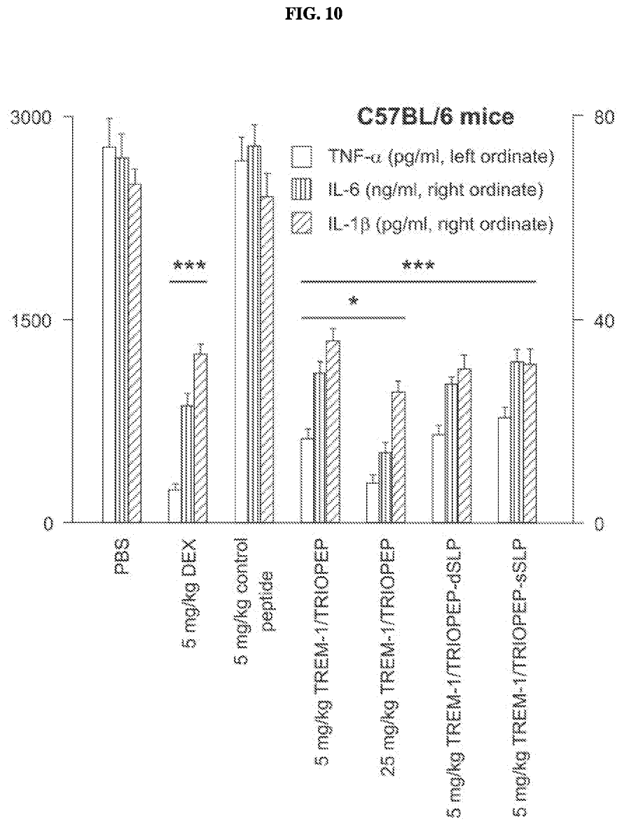

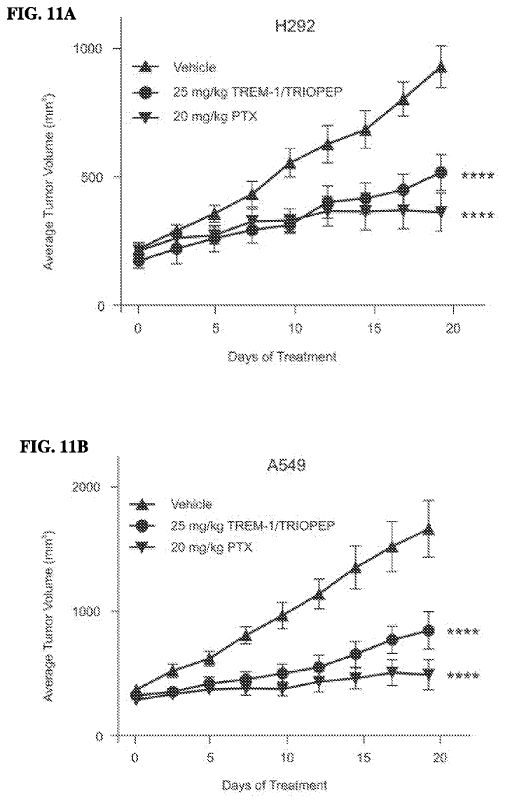

In certain embodiments, FIGS. 8 and 10 demonstrate that TREM-1/TRIOPEP in free and SLP-bound forms inhibits TREM-1 function as shown by reduction of TREM-1-mediated release of pro-inflammatory cytokines, both in vitro (FIG. 8) and in vivo (in serum) (FIG. 10).

While not being bound to any particular theory, it is believed that this indicates that similarly to TREM-1-inhibitory peptide GF9 (see e.g., Sigalov. Int Immunopharmacol 2014, 21:208-219; Shen and Sigalov. Mol Pharm 2017, 14:4572-4582; and Shen and Sigalov. J Cell Mol Med 2017, 21:2524-2534, each of which is herein incorporated by reference in its entirety), TREM-1-related trifunctional peptides can reach their site of action from both outside (free TREM-1/TRIOPEP) and inside (SLP-bound TREM-1/TRIOPEP) the cell. It is also believed that upon administration, free TREM-1/TRIOPEP may form LP in vivo and/or interact with native lipoproteins, resulting in formation of HDL-mimicking LP. In one embodiment, these LP may further target the cells of interest delivering their content to the areas of interest in a body.

FIG. 8 presents the exemplary data showing suppression of tumor necrosis factor-alpha (TNF-alpha), interleukin (IL)-6 and IL-1beta production by lipopolysaccharide (LPS)-stimulated macrophages incubated for 24 h at 37° C. with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. ***, P=0.0001 to 0.001 as compared with medium-treated LPS-challenged macrophages.
FIG. 10 presents the exemplary data showing suppression of tumor necrosis factor-alpha (TNF-alpha), interleukin (IL)-6 and IL-1beta production in mice at 90 min post lipopolysaccharide (LPS) challenge treated 1 h before LPS challenge with phosphate-buffer saline (PBS), dexamethasone (DEX), control peptide and with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. Control peptide represents an equimolar mixture of two peptides, each of them comprising two amino acid domains A and B where domain A represents a non-functional 9 amino acids-long sequence of the TREM-1 inhibitory therapeutic peptide sequence wherein, Lys₅ is substituted with Ala₅, whereas domain B is a sulfoxidized methionine residue-containing 22 amino acids-long apolipoprotein A-I helix 4 or 6 peptide sequence, respectively. *, P=0.01 to 0.05 as compared with animals treated with 5 mg/kg TRIOPEP in free form; ***, P=0.0001 to 0.001 as compared with PBS-treated animals.

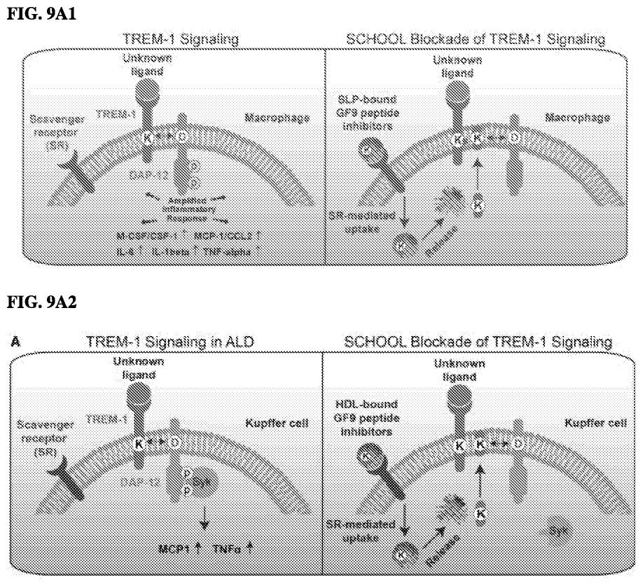

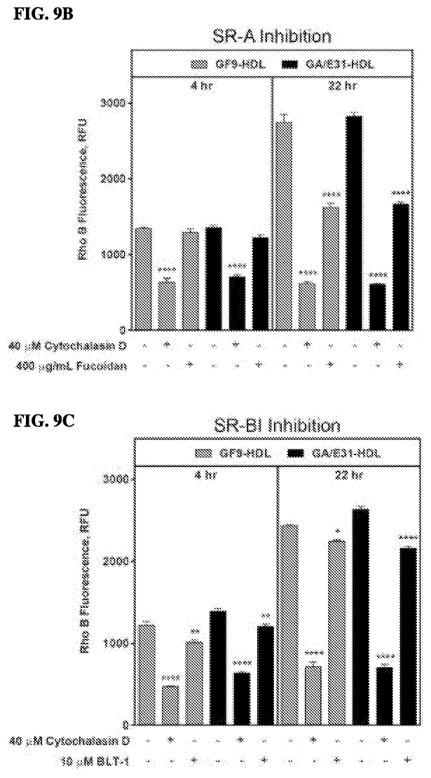

While not being bound to any particular theory, it is believed that increased uptake described herein, is mediated by macrophage scavenger receptors (SR) including, but not limiting to, SR-A and SR-B1 (see FIG. 9A1,A2-C). While not being bound to any particular theory, it is believed that in one embodiment, this colocalization is accompanied by a specific disruption of intramembrane interactions between TREM-1 and DAP-12 by the TREM-1-related trifunctional peptide of the present invention (see FIG. 9A), resulting in ligand-independent inhibition of TREM-1 upon ligand binding as described in Shen and Sigalov. Mol Pharm 2017, 14:4572-4582 and Shen and Sigalov. J Cell Mol Med 2017, 21:2524-2534, each of which is herein incorporated by reference in its entirety.

FIG. 9A1-A2-C shows schematic representations of activation of the TREM-1/DAP12 receptor complex expressed on macrophages and presents the exemplary data showing that scavenger receptors SR-A and SR-B1 mediate the macrophage endocytosis of GF9-sSLP (GF9-HDL) and GA/E31-HDL (TREM-1/TRIOPEP-sSLP). (FIG. 9A) Schematic representation of TREM-1 signaling and the SCHOOL mechanism of TREM-1 blockade. (FIG. 9A1, left panel) Activation of the TREM-1/DAP12 receptor complex expressed on macrophages leads to phosphorylation of the DAP12 cytoplasmic signaling domain and subsequent downstream inflammatory cytokine response (left panel). SR-mediated endocytosis of sSLP-bound GF9, GA31 and GE31 peptide inhibitors by macrophages results in the release of GF9 or GA31 and GE31 into the cytoplasm, which self-penetrate into the cell membrane and block intramembrane interactions between TREM-1 and DAP12, thereby preventing DAP12 phosphorylation and downstream signaling cascade (FIG. 9A1, right panel).
FIG. 9A2, left panel shows schematic representations of activation of the TREM-1/DAP12 receptor complex expressed on Kupffer cells leads to phosphorylation of the DAP12 cytoplasmic signaling domain, subsequent SYK recruitment, and the downstream inflammatory cytokine response. (FIG. 9A2, right panel) SR-mediated endocytosis of HDL-bound GF9 peptide inhibitors by Kupffer cells results in the release of GF9 (GA31 or GE31) into the cytoplasm; GF9 self-penetrates the cell membrane and blocks intramembrane interactions between TREM-1 and DAP12, thereby preventing DAP12 phosphorylation and the downstream signaling cascade. FIG. 9B-9C Macrophage endocytosis of GF9-sSLP (GF9-HDL) and GA/E31-HDL (TREM-1/TRIOPEP-sSLP) in vitro is SR-mediated in a time-dependent manner and is largely driven by SR-A (FIG. 9B, FIG. 9C). As described in the Materials and Methods, J774 macrophages were cultured at 37° C. overnight with medium. Prior to uptake of GF9-HDL and GA/E31-HDL, cells were treated for 1 hour at 37° C. with 40 μM cytochalasin D and either (FIG. 9B) 400 μg/mL fucoidan or (FIG. 9C) 10 μM BLT-1, as indicated. Cells were then incubated for either 4 hours or 22 hours with medium containing 2 μM rhodamine B (rho B)-labeled GF9-sSLP (gray bars) or TREM-1/TRIOPEP-sSLP (black bars), respectively. Cells were lysed, and rho B fluorescence intensities of lysates were measured and normalized to the protein content. Results are expressed as mean±SEM (n=3); *P≤0.05; **P≤0.01; ****P≤0.0001 versus uptake of GF9-HDL and GA/E31-HDL in the absence of inhibitor. Abbreviations: D, DAP12; DAP12, DNAX activation protein of 12 kDa; K, Kupffer cell; RFU, relative fluorescence units; SCHOOL, signaling chain homo-oligomerization.

In certain embodiments, FIGS. 11A-B-14 demonstrate that TREM-1/TRIOPEP in free and SLP-bound forms inhibits tumor growth, reduces infiltration of macrophages into the tumor in mouse models of NSCLC and PC and is well-tolerated by cancer mice during the treatment period (see also Shen and Sigalov. Mol Pharm 2017, 14:4572-4582, each of which is herein incorporated by reference in its entirety).

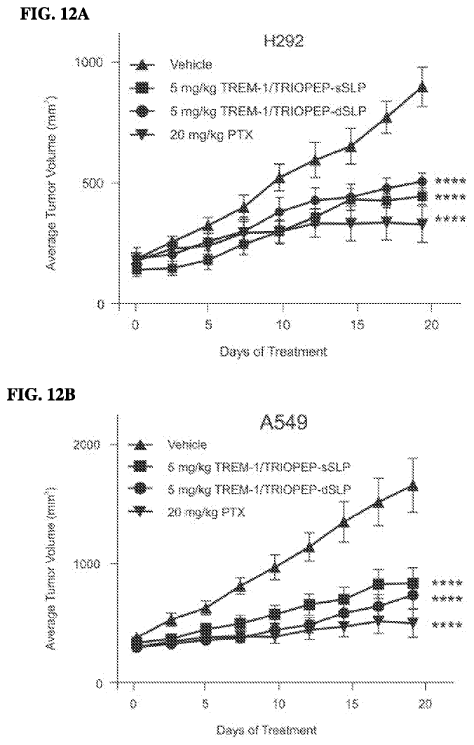

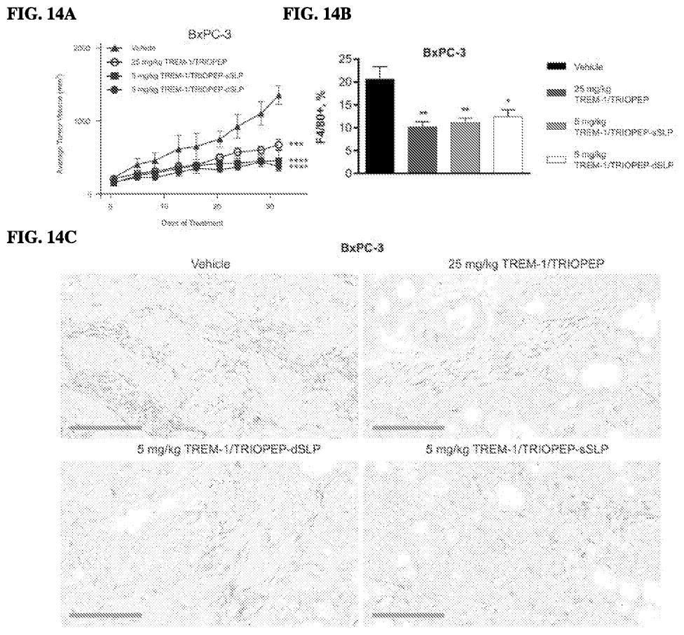

FIG. 11A-B presents the exemplary data showing inhibition of tumor growth in the human non-small cell lung cancer H292 (FIG. 11A) and A549 (FIG. 11B) xenograft mice treated with an equimolar mixture of the sulfoxidized methionine residue-containing TREM-1-related trifunctional peptides (TREM-1/TRIOPEP) GA 31 and GE 31 in free form. PTX, paclitaxel. ****, P≤0.0001 as compared with vehicle-treated animals.
FIG. 12A-B presents the exemplary data showing inhibition of tumor growth in the human non-small cell lung cancer H292 (FIG. 12A) and A549 (FIG. 12B) xenograft mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.
FIG. 13 presents the exemplary data showing average tumor weights in the A549 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.
FIG. 14A-C presents the exemplary data showing inhibition of tumor growth (FIG. 14A) and TREM-1 blockade-mediated suppression of intratumoral macrophage infiltration (FIG. 14B, FIG. 14C) in the human pancreatic cancer BxPC-3 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. (B) F4/80 staining. Results are expressed as the mean±SEM (n=4 mice per group). *, p<0.05; **, p<0.01, ****, p<0.0001 (versus vehicle). (FIG. 14C) Representative F4/80 images from BxPC-3-bearing mice treated using different free and sSLP-bound SCHOOL TREM-1 inhibitory GF9 sequences including TREM-1/TRIOPEP-sSLP. Scale bar=200 μm.

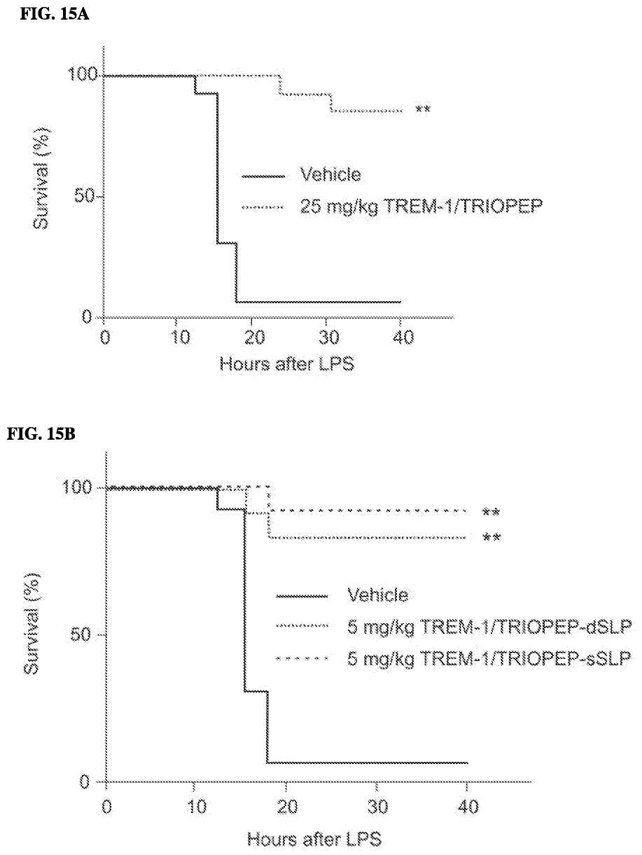

In embodiment, FIG. 15 demonstrates that TREM-1/TRIOPEP in free and SLP-bound forms significantly prolongs survival in mice with lipopolysaccharide (LPS)-induced septic shock.

FIG. 15A-B presents the exemplary data showing improved survival of lipopolysaccharide (LPS)-challenged mice treated with an equimolar mixture of the sulfoxidized methionine residue-containing TREM-1-related trifunctional peptides (TREM-1/TRIOPEP) GA 31 and GE 31 in free form (FIG. 15A) or incorporated into synthetic lipopeptide particles (SLP) particles of discoidal (TREM-1/TRIOPEP-dSLP) and spherical (TREM-1/TRIOPEP-sSLP) morphology. FIG. 15B. **, P=0.001 to 0.01 as compared with vehicle-treated animals.

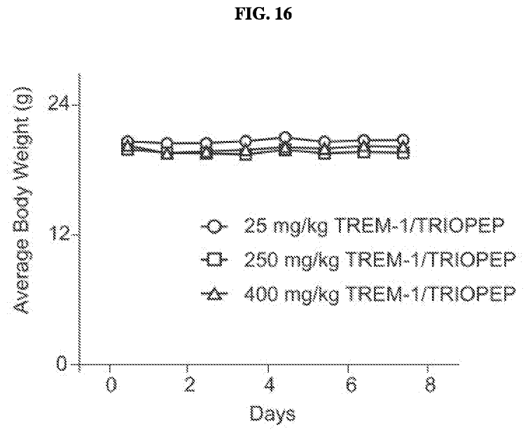

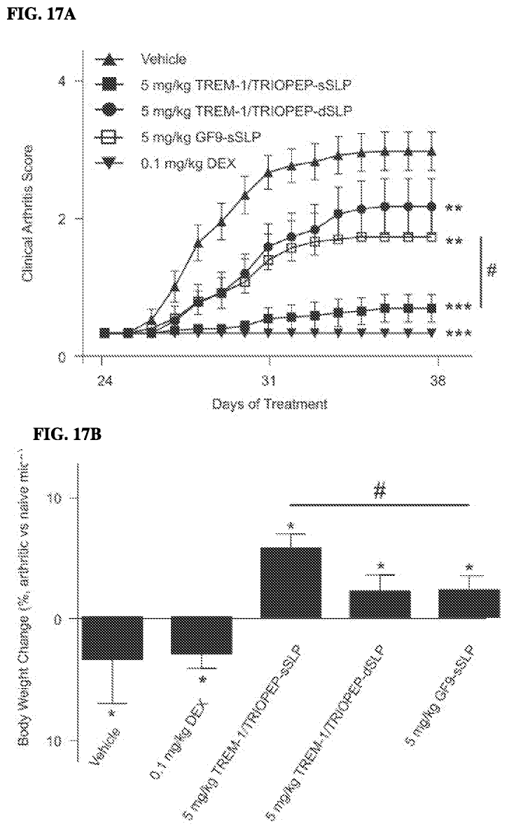

In one embodiment, FIG. 16 shows that TREM-1/TRIOPEP is non-toxic in healthy mice at least up to 400 mg/kg.

FIG. 16 presents exemplary data showing average weights of healthy C57BL/6 mice treated with increasing concentrations of an equimolar mixture of the sulfoxidized methionine residue-containing TREM-1-related trifunctional peptides (TREM-1/TRIOPEP) GA 31 and GE 31 in free form.

In one embodiment, FIG. 17 demonstrates that TREM-1/TRIOPEP in free and SLP-bound form ameliorates arthritis in mice with collagen-induced arthritis (CIA) and is well-tolerated by arthritic mice during the treatment period of 2 weeks (see Shen and Sigalov. J Cell Mol Med 2017, 21:2524-2534, each of which is herein incorporated by reference in its entirety). FIG. 17A-B presents the exemplary data showing average clinical arthritis score (FIG. 17A) and mean body weight (BW) changes (FIG. 17B) calculated as a percentage of the difference between beginning (day 24) and final (day 38) BWs of the collagen-induced arthritis (CIA) mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. DEX, dexamethasone. *, p<0.05, **, p<0.01; ***, p<0.001 as compared with vehicle-treated or naive animals.

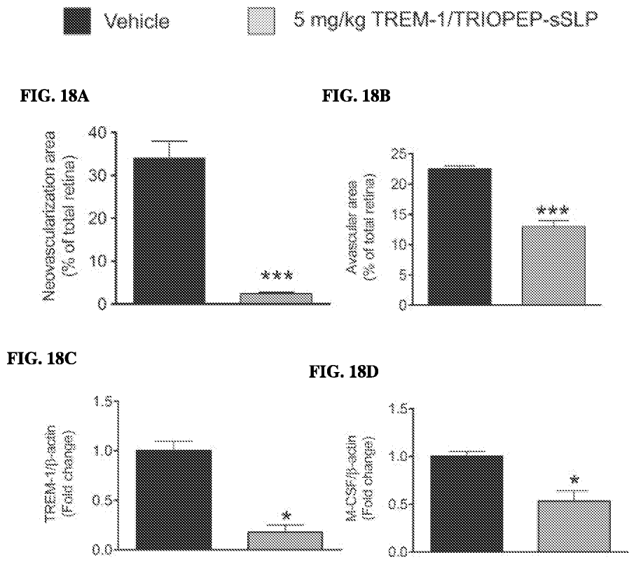

In certain embodiments, FIG. 18 demonstrates that TREM-1/TRIOPEP-sSLP prevents pathological RNV in mice with oxygen-induced retinopathy and is well-tolerated by these mice during the treatment period (see Rojas, et al. Biochim Biophys Acta 2018, 1864:2761-2768, each of which is herein incorporated by reference in its entirety).

FIG. 18A-D presents the exemplary data showing reduction of pathological retinal neovascularization area (FIG. 18A), avascular area (FIG. 18B) and retinal TREM-1 (FIG. 18C) and M-CSF/CSF-1 (FIG. 18D) expression in the retina of the mice with oxygen-induced retinopathy (OIR) treated with an equimolar mixture of the sulfoxidized methionine residue-containing TREM-1-related trifunctional peptides (TREM-1/TRIOPEP) GA 31 and GE 31 incorporated into synthetic lipopeptide particles (TREM-1/TRIOPEP-SLP) particles of spherical morphology (TREM-1/TRIOPEP-sSLP). ***, p<0.001 as compared with vehicle-treated animals.

As described in Stukas, et al. J Am Heart Assoc 2014, 3:e001156, herein incorporated by reference in its entirety, systemically administered human apo A-I accumulates in murine brain. It is also known that transcytosis of HDL in brain microvascular endothelial cells is mediated by SRBI (see Fung, et al. Front Physiol 2017, 8:841, herein incorporated by reference in its entirety). However, until tested as described herein, it was not known that a self-assembled SLP of the present invention comprising a trifunctional peptide was capable of crossing the BBB.

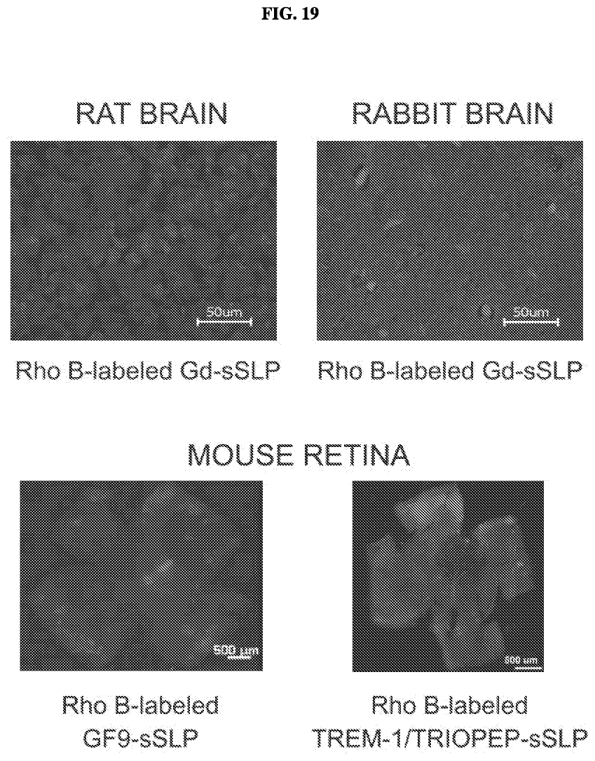

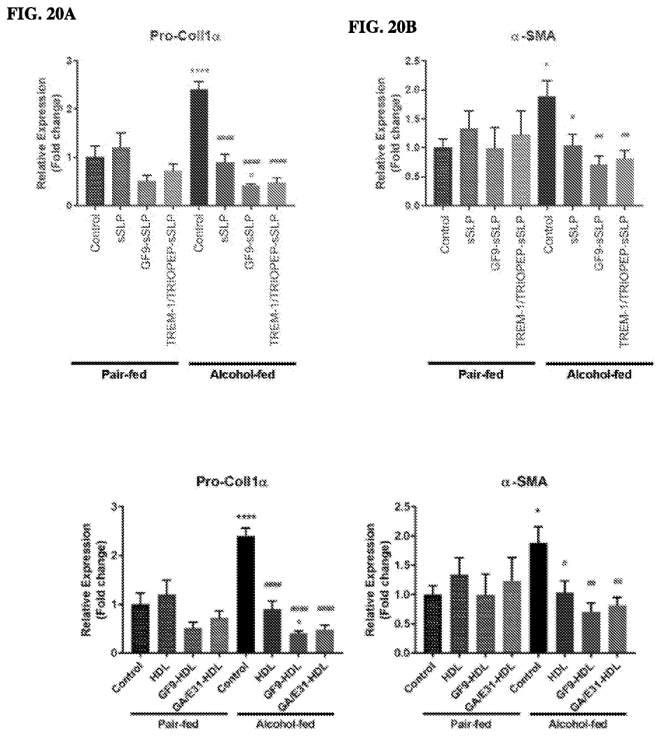

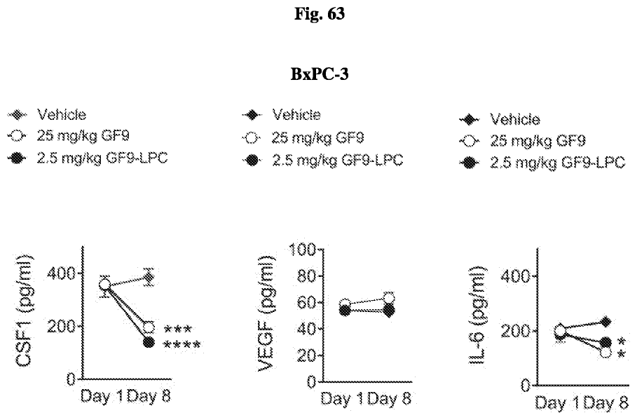

In certain embodiments, FIG. 19 shows that the self-assembled SLP of the present invention may cross the BBB, BRB and BTB, thus delivering their constituents including but not limiting to, TREM-1/TRIOPEP, GF9, GA31 and GE31, to the areas of interest in the brain, retina and tumor. In certain embodiments, FIG. 63 demonstrates that the fluorescently labeled sSLP described herein may cross the BBB, BRB and BTB, thus delivering their constituents including but not limiting to, GBCA imaging probe to the areas of interest in the brain, retina and tumor.

While not being bound to any particular theory, it is believed that the brain-, retina-, and tumor-penetrating capabilities of these SLP can be mediated by interaction of SRBI with the domain B amino acid sequences that correspond to the sequences of human apo A-I helices 4 and/or 6 (see e.g. Liu, et al. J Biol Chem 2002, 277:21576-21584, herein incorporated by reference in its entirety).

In certain embodiments, these capabilities of the peptides and compositions of the present invention can be used to diagnose, treat and/or prevent cancers (including brain cancer), diabetic retinopathy and retinopathy of prematurity, neurodegenerative diseases including Alzheimer's, Parkinson's and Huntington's diseases and other diseases and conditions where delivery of the peptides and compositions of the invention to the brain, retina and/or tumor is needed.

FIG. 19 presents exemplary data showing penetration of the blood-brain barrier (BBB) and blood-retinal barrier (BRB) by systemically (intraperitoneally) administered rhodamine B-labeled spherical self-assembled particles (sSLP) that contain Gd-containing contrast agent (Gd-sSLP) for magnetic resonance imaging (MRI), TREM-1 inhibitory peptide GF9 (GF9-sSLP) or an equimolar mixture of the sulfoxidized methionine residue-containing TREM-1-related trifunctional peptides GA 31 and GE 31 (TREM-1/TRIOPEP-sSLP).

A mouse model of ALD mimics the early phase of the human disease, yet mRNA levels of early fibrosis markers Pro-Colla and a-SMA were significantly increased in alcohol-fed mice compared to PF controls in the whole-liver samples (FIG. 20A-B). Induction of these makers was remarkably attenuated in the vehicle-treated group and, importantly, further decreased by the TREM-1 inhibitory formulations used (FIG. 20A-B).

FIG. 20A-B presents exemplary data showing TREM-1/TRIOPEP-sSLP suppresses the expression of fibrinogenesis marker molecules, FIG. 20A Pro-Collagen 1α and FIG. 20B α-Smooth Muscle Actin, at the RNA level, as measured in whole-liver lysates of mice with (alcohol-fed) and without (pair-fed) alcoholic liver disease (ALD).
* indicates significance level compared to the non-treated pair-fed (PF) group; #indicates significance level compared to the non-treated alcohol-fed group. o indicates significance level compared to the vehicle-treated alcohol-fed group. The significant levels are as follows: *, 0.05≥P≥0.01; **, 0.01≥P≥0.001; ***, 0.001≥P≥0.0001; ****, P≤0.0001.

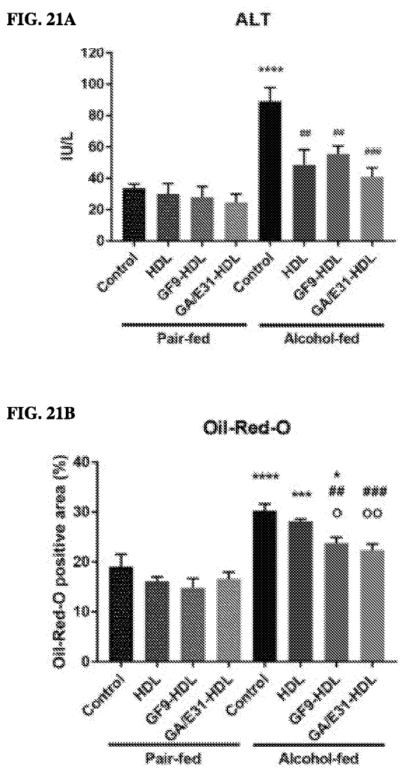

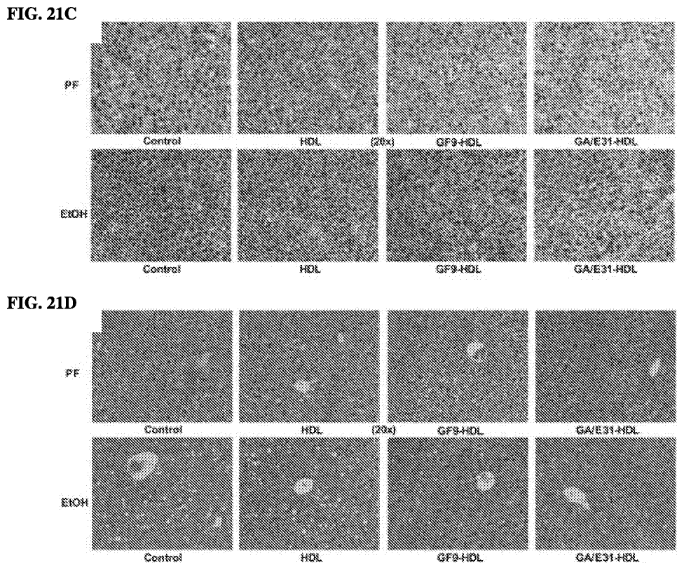

TREM-1 inhibitor effects were evaluated on hepatocyte damage and steatosis in liver. Serum ALT levels obtained during week 5 of the alcohol feeding showed significant increases in alcohol-fed mice compared to PF controls. This ALT increase was attenuated in both TREM-1 inhibitor-treated groups, indicating attenuation of liver injury (FIG. 21A). Surprisingly, vehicle treatment (HDL) also showed a similar protective effect (FIG. 21A).

Consistent with steatosis, we found a significant increase in Oil Red O staining in livers of alcohol-fed mice compared to PF controls (FIG. 21C). Oil Red O (FIG. 21B-D) and H&£ (FIG. 21D) staining revealed attenuation of steatosis in the alcohol-fed TREM-1 inhibitor-treated mice compared to both untreated and vehicle (HDL)-treated alcohol-fed groups (FIG. 21B-D).

FIG. 21A-D presents exemplary data showing that TREM-1/TRIOPEP-sSLP suppresses the production of alanine aminotransferase (ALT) in mice with alcoholic liver disease (ALD), as measured in serum of mice with (alcohol-fed) and without (pair-fed) ALD, in addition to improving indicators of liver disease and inflammation. * indicates significance level compared to the alcohol-fed group treated with vehicle-synthetic lipopeptide particles of spherical morphology that contained an equimolar mixture of PE22 and PA22 (sSLP) but no TREM-1 inhibitory peptide GF9. #indicates significance level compared to the non-treated alcohol-fed group. Liver damage after 5 weeks of alcohol feeding and effect of TREM-1 pathway inhibition in a mouse model of ALD. sSLP, 5 mg/kg treatment of TREM-1 peptide vs. TREM-1/TRIOPEP-sSLP. Cheek blood and livers were harvested at death. (FIG. 21A) Serum ALT levels were measured using a kinetic method. Exemplary data showing TREM-1/TRIOPEP-sSLP suppresses alanine aminotransferase in serum of alcohol fed mice over TREM-1 peptide alone. (FIG. 21B-D) Liver sections were stained with (B,C) Oil Red O and (FIG. 21D) H&E staining, and the lipid content was analyzed by ImageJ (FIG. 21B). * indicates significance level compared to the nontreated PF group; * indicates significance level compared to the nontreated alcohol-fed group; ⁰ indicates significance level compared to the vehicle-treated alcohol-fed group. The numbers of the symbols sign the significant levels as the following: **^{o}P≤0.05; ^{##/oo}P≤0.01; *″^{/###}P≤0.001; ****P<0.0001. ***, 0.001≥P≥0.0001; ##, 0.01≥P≥0.001.

B. TCR-Related Trifunctional Peptides

The T-cell receptor (TCR)-CD3 complex plays a role in T-cell differentiation, in protecting the organism from infectious agents, and in the function of T-cells. The TCR is a complex of a heterodimer of TCRa and TCRb chains, which are responsible for antigen recognition and interaction with the major histocompatibility complex (MHC) molecules of antigen-presenting cells, and CD3d, CD3g, CD3e and CD3z chains, which are responsible for transmembrane signal transduction (see e.g., Manolios, et al. Cell Adh Migr 2010, 4:273-283; Sigalov. Adv Protein Chem Struct Biol 2018, 111:61-9; Sigalov. US 20130039948; Manolios. U.S. Pat. No. 6,057,294; Manolios. U.S. Pat. No. 7,192,928; Manolios. US 20100267651; and Manolios, et al. US 20120077732, each of which is herein incorporated by reference in its entirety).

The preferred TCR-related peptides and compositions of this class comprise the domain A comprising the TCR modulatory peptide sequences designed using a well-known in the art novel model of cell receptor signaling, the Signaling Chain HOmoOLigomerization model, capable of modulating TCR (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. PLoS Pathog 2009, 5:e1000404; Shen and Sigalov. Sci Rep 2016, 6:28672; Sigalov. US 20130039948, each of which is herein incorporated by reference in its entirety). The preferred peptides and compositions of this class further comprise the domain B comprising at least one modified or unmodified amphipathic alpha helical peptide fragment. As described above, the inclusion of an amphipathic amino acid sequences aids the assistance in the ability to interact with native lipoproteins in a bloodstream in vivo and to form naturally long half-life lipopeptide/lipoprotein particles LP. It further aids the ability to provide targeted delivery to the sites of interest. It further aids the ability to cross the BBB, BRB and BTB.

The preferred TCR-related peptides and compositions of this class comprise the domain A comprising the TCR modulatory peptide sequences designed using a well-known in the art novel model of cell receptor signaling, the Signaling Chain HOmoOLigomerization model, capable of modulating TCR (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. PLoS Pathog 2009, 5:e1000404; Shen and Sigalov. Sci Rep 2016, 6:28672; Sigalov. US 20130039948, each of which is herein incorporated by reference in its entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative TCR-related trifunctional peptides: TCR/TRIOPEP MA32 (MWKTPTLKYFPLGEEMRDRARAHVDALRTHLA) (SEQ ID NO. 11), TCR/TRIOPEP ME32 (MWKTPTLKYFPYLDDFQKKWQEEMELYRQKVE) (SEQ ID NO. 12), TCR/TRIOPEP GA36 (GARSMTLTVQARQLPLGEEMRDRARAHVDALRTHLA) (SEQ ID NO 13), TCR/TRIOPEP GE36 (GARSMTLTVQARQLPYLDDFQKKWQEEMELYRQKVE) (SEQ ID NO 14), TCR/TRIOPEP GA32 (GVLRLLLFKLPLGEEMRDRARAHVDALRTHLA) (SEQ ID NO 15), and TCR/TRIOPEP GE32 (GVLRLLLFKLPYLDDFQKKWQEEMELYRQKVE) (SEQ ID NO 16). While not being bound to any particular theory, it is believed that in one embodiment, these peptides colocalize with TCR in the cell membrane and selectively disrupt intramembrane interactions of TCRa chain with the CD3ed heterodimer and CD3zz homodimer, resulting to specific ligand-independent inhibition of TCR upon antigen stimulation (see e.g. Sigalov. Self Nonself 2010, 1:4-39.; Sigalov. Self Nonself 2010, 1:192-224; and Sigalov. Adv Protein Chem Struct Biol 2018, 111:61-99, each of which is herein incorporated by reference in its entirety). In one embodiment, methionine residues of TCR/TRIOPEP peptides are modified.

In certain embodiments, TABLE 2 demonstrates the following structures of representative TCR-related trifunctional peptides: TCR/TRIOPEP LA32 (LGKATLYAVLPLGEEMRDRARAHVDALRTHLA) (SEQ ID NO. 17) and TCR/TRIOPEP LE32 (LGKATLYAVLPYLDDFQKKWQEEMELYRQKVE) (SEQ ID NO. 18). While not being bound to any particular theory, it is believed that in one embodiment, these peptides colocalize with TCR in the cell membrane and selectively disrupt intramembrane interactions of TCRb chain with the CD3eg heterodimer, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in its entirety).

In certain embodiments, TABLE 2 demonstrates the following structures of representative TCR-related trifunctional peptides: TCR/TRIOPEP YA32 (YLLDGILFIYPLGEEMRDRARAHVDALRTHLA) (SEQ ID NO. 19) and TCR/TRIOPEP YE32 (YLLDGILFIYPYLDDFQKKWQEEMELYRQKVE) (SEQ ID NO. 20). While not being bound to any particular theory, it is believed that in one embodiment, these peptides colocalize with TCR in the cell membrane and selectively disrupt intramembrane interactions of CD3zz homodimer with TCRa chain, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99).

In certain embodiments, TABLE 2 demonstrates the following structures of representative TCR-related trifunctional peptides: TCR/TRIOPEP IA32 (IIVTDVIATLPLGEEMRDRARAHVDALRTHLA) (SEQ ID NO. 21) and TCR/TRIOPEP IE32 (IIVTDVIATLPYLDDFQKKWQEEMELYRQKVE) (SEQ ID NO. 22). While not being bound to any particular theory, it is believed that in one embodiment, these peptides colocalize with TCR in the cell membrane and selectively disrupt intramembrane interactions of CD3ed heterodimer with TCRa chain, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in its entirety).

In certain embodiments, TABLE 2 demonstrates the following structures of representative TCR-related trifunctional peptides: TCR/TRIOPEP FA32 (FLFAEIVSIFPLGEEMRDRARAHVDALRTHLA) (SEQ ID NO. 23) and TCR/TRIOPEP FE32 (FLFAEIVSIFPYLDDFQKKWQEEMELYRQKVE) (SEQ ID NO. 24). While not being bound to any particular theory, it is believed that in one embodiment, these peptides colocalize with TCR in the cell membrane and selectively disrupt intramembrane interactions of CD3eg heterodimer with TCRb chain, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in its entirety).

In certain embodiments, TABLE 2 demonstrates the following structures of representative TCR-related trifunctional peptides: TCR/TRIOPEP IA32e (IVIVDICITGPLGEEMRDRARAHVDALRTHLA) (SEQ ID NO. 25) and TCR/TRIOPEP IE32e (IVIVDICITGPYLDDFQKKWQEEMELYRQKVE) (SEQ ID NO. 26). While not being bound to any particular theory, it is believed that in one embodiment, these peptides colocalize with TCR in the cell membrane and selectively disrupt intramembrane interactions of CD3eg and CD3ed heterodimers with TCRb and TCRa chains, respectively, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in its entirety).

In one embodiment, methionine residues of TCR/TRIOPEP peptides are modified.

In certain embodiments, the capability of the TCR-related peptides and compounds of the present invention including but not limiting to those described above, to inhibit TCR can be used to treat and/or prevent TCR-related diseases and conditions including but not limiting to, allergic diathesis e.g. delayed type hypersensitivity, contact dermatitis; autoimmune disease e.g. systemic lupus erythematosus, rheumatoid arthritis, multiple sclerosis, diabetes, Guillain-Barre syndrome, Hashimotos disease, pernicious anaemia; gastroenterological conditions e.g. inflammatory bowel disease, Crohn's disease, primary biliary cirrhosis, chronic active hepatitis; skin problems e.g. atopic dermatitis, psoriasis, pemphigus vulgaris; infective disease; respiratory conditions e.g. allergic alveolitis; cardiovascular problems e.g. autoimmune pericarditis; organ transplantation; inflammatory conditions e.g. myositis, ankylosing spondylitis; and any other disorder where T cells are involved/recruited

In certain embodiments, the capability of the TCR-related peptides and compounds of the present invention including but not limiting to those described above, to colocalize with TCR can be used to visualize (image) this receptor and evaluate its expression in the areas of interest. In one embodiment, for this purpose, an imaging probe (e.g. [⁶⁴Cu], see TABLE 2) can be conjugated to the TCR/TRIOPEP sequences In one embodiment, imaging (visualization) of TCR levels using PET and/or other imaging techniques can be used to diagnose TCR-related diseases and conditions as well as to monitor novel therapies for these diseases and conditions.

C. NKG2D-Related Trifunctional Peptides

NKG2D is an activating receptor expressed by natural killer (NK) and T cells. The NKG2D is a complex of an NKG2D chain, which is responsible for ligand recognition, and DAP10 homodimer, which is responsible for transmembrane signal transduction (see e.g.

Sigalov. Trends Pharmacol Sci 2006, 27:518-524; Sigalov. Trends Immunol 2004, 25:583-589; Sigalov. Self Nonself 2010, 1:4-39; and Sigalov. Self Nonself 2010, 1:192-224, each of which is herein incorporated by reference in its entirety). NKG2D ligands show a restricted expression in normal tissues, but they are frequently overexpressed in cancer and infected cells. The binding of NKG2D to its ligands activates NK and T cells and promotes cytotoxic lysis of the cells expressing these molecules. The mechanisms involved in the expression of the ligands of NKG2D play a role in the recognition of stressed cells by the immune system and represent a promising therapeutic target for improving the immune response against cancer or autoimmune disease (see e.g. Gonzalez, et al. Trends Immunol 2008, 29:397-403; Ito, et al. Am J Physiol Gastrointest Liver Physiol 2008, 294:G199-207; Vilarinho, et al. Proc Natl Acad Sci USA 2007, 104:18187-18192; Van Belle, et al. J Autoimmun 2013, 40:66-73; Lopez-Soto, et al. Int J Cancer 2015, 136:1741-1750; and Urso, et al. U.S. Pat. No. 9,127,064, each of which is herein incorporated by reference in its entirety).

The preferred NKG2D-related peptides and compositions of this class comprise the domain A comprising the NKG2D modulatory peptide sequences designed using a well-known in the art novel model of cell receptor signaling, the Signaling Chain HOmoOLigomerization model, capable of modulating NKG2D (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9, each of which is herein incorporated by reference in its entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative NKG2D-related trifunctional peptides: NKG2D/TRIOPEP IA36 (IAVAMGIRFIIMVAPLGEEMRDRARAHVDALRTHLA) (SEQ ID NO. 27) and NKG2D/TRIOPEP IE36 (IAVAMGIRFIIMVAPYLDDFQKKWQEEMELYRQKVE) (SEQ ID NO. 28). While not being bound to any particular theory, it is believed that in one embodiment, these peptides colocalize with NKG2D in the cell membrane and selectively disrupt intramembrane interactions of NKG2D chain with the DNAX-activation protein 10 (DAP-10) signaling homodimer, resulting to specific ligand-independent inhibition of NKG2D upon ligand stimulation (see e.g. Sigalov. Self Nonself 2010, 1:4-39.; Sigalov. Self Nonself 2010, 1:192-224; and Sigalov. Adv Protein Chem Struct Biol 2018, 111:61-99, each of which is herein incorporated by reference in its entirety). In one embodiment, methionine residues of NKG2D/TRIOPEP peptides are modified.

In certain embodiments, the capability of the NKG2D-related peptides and compounds of the present invention including but not limiting to those described above, to inhibit NKG2D can be used to treat and/or prevent NKG2D-related diseases and conditions including but not limiting to, celiac disease, type I diabetes, hepatitis, and rheumatoid arthritis, and any other disorder where NKG2D cells are involved/recruited. In one embodiment, the present invention provides methods and compositions for preventing NK cell-mediated graft rejection.

In certain embodiments, the capability of the NKG2D-related peptides and compounds of the present invention including but not limiting to those described above, to colocalize with NKG2D can be used to visualize (image) this receptor and evaluate its expression in the areas of interest. In one embodiment, for this purpose, an imaging probe (e.g. [⁶⁴Cu], see TABLE 2) can be conjugated to the NKG2D/TRIOPEP sequences In one embodiment, imaging (visualization) of NKG2D levels using PET and/or other imaging techniques can be used to diagnose NKG2D-related diseases and conditions as well as to monitor novel therapies for these diseases and conditions.

D. GPVI-Related Trifunctional Peptides.

In recent years, the central activating platelet collagen receptor, glycoprotein (GP) VI, has emerged as a promising antithrombotic target because its blockade or antibody-mediated depletion in circulating platelets was shown to effectively inhibit experimental thrombosis and thromboinflammatory disease states, such as stroke, without affecting hemostatic plug formation. GPVI is a complex of an GPVI chain, which is responsible for ligand recognition, and FcRg homodimer, which is responsible for transmembrane signal transduction (see e.g. Sigalov. Trends Pharmacol Sci 2006, 27:518-524; Sigalov. Trends Immunol 2004, 25:583-589; Sigalov. Self Nonself 2010, 1:4-39; Sigalov. Self Nonself 2010, 1:192-224; Sigalov. J Thromb Haemost 2007, 5:2310-2312; Sigalov. Expert Opin Ther Targets 2008, 12:677-692; Dutting, et al. Trends Pharmacol Sci 2012, 33:583-590; Ungerer, et al. PLoS One 2013, 8:e71193; Sigalov, U.S. Pat. No. 8,278,271; Sigalov, U.S. Pat. No. 8,614,188, each of which is herein incorporated by reference in its entirety). The binding of GPVI to collagen or other antagonists ligands induces platelet adhesion, activation and aggregation. Platelet activation is a step in the pathogenesis of ischemic cardio- and cerebrovascular diseases, which represent the leading causes of death and severe disability worldwide. Although existing antiplatelet drugs have proved beneficial in the clinic, their use is limited by their inherent effect on primary hemostasis, making the identification of novel pharmacological targets for platelet inhibition a goal of cardiovascular research.

The preferred GPVI-related peptides and compositions of this class comprise the domain A comprising the GPVI modulatory peptide sequences designed using a well-known in the art novel model of cell receptor signaling, the Signaling Chain HOmoOLigomerization model, capable of modulating GPVI (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. J Thromb Haemost 2007, 5:2310-2312; Sigalov. Expert Opin Ther Targets 2008, 12:677-692, each of which is herein incorporated by reference in its entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative GPVI-related trifunctional peptides: GPVI/TRIOPEP GA32 (GNLVRICLGAPLGEEMRDRARAHVDALRTHLA) (SEQ ID NO. 29) and GPVI/TRIOPEP GE32 (GNLVRICLGAPYLDDFQKKWQEEMELYRQKVE) (SEQ ID NO. 30). While not being bound to any particular theory, it is believed that in one embodiment, these peptides colocalize with GPVI in the cell membrane and selectively disrupt intramembrane interactions of GPVI chain with the FcRg signaling homodimer, resulting to specific ligand-independent inhibition of GPVI upon ligand stimulation (see e.g. Sigalov. Self Nonself 2010, 1:4-39.; Sigalov. Self Nonself 2010, 1:192-224; Sigalov. Adv Protein Chem Struct Biol 2018, 111:61-99; Sigalov. J Thromb Haemost 2007, 5:2310-2312; Sigalov. Expert Opin Ther Targets 2008, 12:677-692, each of which is herein incorporated by reference in its entirety). In one embodiment, methionine residues of GPVI/TRIOPEP peptides are modified.

In certain embodiments, the capability of the GPVI-related peptides and compounds of the present invention including but not limiting to those described above, to inhibit GPVI can be used to treat and/or prevent GPVI-related diseases and conditions including but not limiting to, ischemic and thromboinflammatory diseases, and any other disorder where platelets are involved/recruited.

In certain embodiments, the capability of the GPVI-related peptides and compounds of the present invention including but not limiting to those described above, to colocalize with GPVI can be used to visualize (image) this receptor and evaluate its expression in the areas of interest. In one embodiment, for this purpose, an imaging probe (e.g. [⁶⁴Cu], see TABLE 2) can be conjugated to the GPVI/TRIOPEP sequences In one embodiment, imaging (visualization) of GPVI levels using PET and/or other imaging techniques can be used to diagnose GPVI-related diseases and conditions as well as to monitor novel therapies for these diseases and conditions.

E. DAP-10- and DAP-12-Related Trifunctional Peptides

The DAP10 and DAP12 signaling subunits are highly conserved in evolution and associate with a large family of receptors in hematopoietic cells, including dendritic cells, plasmacytoid dendritic cells, neutrophils, basophils, eosinophils, mast cells, monocytes, macrophages, natural killer cells, and some B and T cells. Some receptors are able to associate with either DAP10 or DAP12, which contribute unique intracellular signaling functions. DAP-10- and DAP-12-associated receptors have been shown to recognize both host-encoded ligands and ligands encoded by microbial pathogens, indicating that they play a role in innate immune responses. See e.g. Lanier. Immunol Rev 2009, 227:150-160; Sigalov. Trends Pharmacol Sci 2006, 27:518-524; Sigalov. Trends Immunol 2004, 25:583-589; Sigalov. Self Nonself 2010, 1:4-39; Sigalov. Self Nonself 2010, 1:192-224, each of which is herein incorporated by reference in its entirety.

The preferred DAP-10 and DAP-12-related peptides and compositions of this class comprise the domain A comprising the DAP-10 or DAP-12 modulatory peptide sequences, respectively, designed using a well-known in the art novel model of cell receptor signaling, the Signaling Chain HOmoOLigomerization model, capable of modulating DAP-10- and DAP-12-associated receptors (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9, each of which is herein incorporated by reference in its entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative DAP-10-related trifunctional peptides: DAP-10/TRIOPEP LA32 (LVAADAVASLPLGEEMRDRARAHVDALRTHLA) (SEQ ID NO. 33) and DAP-10/TRIOPEP LE32 (LVAADAVASLPYLDDFQKKWQEEMELYRQKVE) (SEQ ID NO. 34).

While not being bound to any particular theory, it is believed that in one embodiment, these peptides colocalize with DAP-10-associated cell receptors in the cell membrane and selectively disrupt intramembrane interactions of the receptor with the DAP-10 signaling homodimer, resulting to specific ligand-independent inhibition of the receptor upon ligand stimulation (see e.g. Sigalov. Self Nonself 2010, 1:4-39.; Sigalov. Self Nonself 2010, 1:192-224; Sigalov. Adv Protein Chem Struct Biol 2018, 111:61-99, each of which is herein incorporated by reference in its entirety). In one embodiment, methionine residues of DAP-10/TRIOPEP peptides are modified.

In certain embodiments, TABLE 2 demonstrates the following structures of representative DAP-12-related trifunctional peptides: DAP-12/TRIOPEP VA32 (VMGDLVLTVLPLGEEMRDRARAHVDALRTHLA) (SEQ ID NO. 31) and DAP-12/TRIOPEP VE32 (VMGDLVLTVLPYLDDFQKKWQEEMELYRQKVE) (SEQ ID NO. 32). While not being bound to any particular theory, it is believed that in one embodiment, these peptides colocalize with DAP-12-associated cell receptors in the cell membrane and selectively disrupt intramembrane interactions of the receptor with the DAP-12 signaling homodimer, resulting to specific ligand-independent inhibition of the receptor upon ligand stimulation (see e.g. Sigalov. Self Nonself 2010, 1:4-39.; Sigalov. Self Nonself 2010, 1:192-224; Sigalov. Adv Protein Chem Struct Biol 2018, 111:61-99, each of which is herein incorporated by reference in its entirety). In one embodiment, methionine residues of DAP-12/TRIOPEP peptides are modified.

In certain embodiments, the capability of the DAP-10- and DAP-12-related peptides and compounds of the present invention including but not limiting to those described above, to inhibit the DAP-10- and DAP-12-associated receptors, respectively, can be used to treat and/or prevent any diseases and conditions where these receptors are involved.

In certain embodiments, the capability of the DAP-10- and DAP-12-related peptides and compounds of the present invention including but not limiting to those described above, to colocalize with the DAP-10- and DAP-12-associated receptors, respectively, can be used to visualize (image) these receptors and evaluate their expression in the areas of interest. In one embodiment, for this purpose, an imaging probe (e.g. [⁶⁴Cu], see TABLE 2) can be conjugated to the DAP-10/TRIOPEP and DAP-12/TRIOPEP sequences. In one embodiment, imaging (visualization) of levels of the DAP-10- and DAP-12-associated receptors using PET and/or other imaging techniques can be used to diagnose any diseases and conditions where these receptors are involved as well as to monitor novel therapies for these diseases and conditions.

F. EGFR-Related Trifunctional Peptides.

The epidermal growth factor (EGF) receptor (EGFR) family, or ErbB family, is the best studied example of oncogenic receptor tyrosine kinases (RTKs). HER2/ErbB2 is overexpressed on the surface of 25-30% of breast cancer cells, and it has been associated with a high risk of relapse and death. EGFR amplification and mutations have been associated with many carcinomas. In particular, the EGFR pathway appears to play a role in pancreatic carcinoma. See e.g. Overholser, et al. Cancer 2000, 89:74-82; Bennasroune, et al. Mol Biol Cell 2004, 15:3464-3474; Sigalov. Trends Pharmacol Sci 2006, 27:518-524; Sigalov. Trends Immunol 2004, 25:583-589; Sigalov. Self Nonself 2010, 1:4-39; Sigalov. Self Nonself 2010, 1:192-224. Short hydrophobic peptides corresponding to the transmembrane domains of EGFR, ErB2 and insulin receptors inhibit specifically the autophosphorylation and signaling pathway of their cognate receptor (see Bennasroune, et al. Mol Biol Cell 2004, 15:3464-3474).

The preferred EGFR-related peptides and compositions of this class comprise the domain A comprising the EGFR modulatory peptide sequences, designed using a well-known in the art novel model of cell receptor signaling, the Signaling Chain HOmoOLigomerization model, capable of modulating EGFR (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9, each of which is herein incorporated by reference in its entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative EGFR-related trifunctional peptides: EGFR/TRIOPEP SA47 (SIATGMVGALLLLLVVALGIGLFMRPLGEEMRDRARAHVDALRTHLA) (SEQ ID NO. 35) and EGFR/TRIOPEP SE47 (SIATGMVGALLLLLVVALGIGLFMRPYLDDFQKKWQEEMELYRQKVE) (SEQ ID NO. 36). While not being bound to any particular theory, it is believed that in one embodiment, these peptides colocalize with EGFR in the cell membrane and selectively disrupts intramembrane interactions between the receptors, resulting to specific ligand-independent inhibition of the receptor (see e.g. Sigalov. Self Nonself 2010, 1:4-39.; Sigalov. Self Nonself 2010, 1:192-224; Sigalov. Adv Protein Chem Struct Biol 2018, 111:61-99, each of which is herein incorporated by reference in its entirety). In one embodiment, methionine residues of EGFR/TRIOPEP peptides are modified.

In certain embodiments, the capability of the EGFR and/or ErB-related peptides and compounds of the present invention including but not limiting to those described above, to inhibit the receptors of the EGFR and/or ErB receptor families, respectively, can be used to treat and/or prevent any diseases and conditions where these receptors are involved.

In certain embodiments, the capability of the EGFR- and/or ErB-related peptides and compounds of the present invention including but not limiting to those described above, to colocalize with the receptors of the EGFR and/or ErB receptor families can be used to visualize (image) these receptors and evaluate their expression in the areas of interest. In one embodiment, for this purpose, an imaging probe (e.g. [⁶⁴Cu]) can be conjugated to the EGFR/TRIOPEP sequence. In one embodiment, imaging (visualization) of levels of the receptors of the EGFR and/or ErB receptor families using PET and/or other imaging techniques can be used to diagnose any diseases and conditions where these receptors are involved as well as to monitor novel therapies for these diseases and conditions.

G. Additional Trifunctional Peptides

Additional therapeutic peptide sequences and/or other therapeutic agents can comprise the domain A of the peptides and compositions of the present invention. Additional examples are provided in, for e.gs., Vlieghe, et al. Drug Discov Today 2010, 15:40-56; Tsung, et al. Shock 2007, 27:364-369; Chang, et al. PLoS One 2009, 4:e4171; Tjin Tham Sjin, et al. Cancer Res 2005, 65:3656-3663; Ladetzki-Baehs, et al. Endocrinology 2007, 148:332-336; Khan, et al. Hum Immunol 2002, 63:1-7; Banga. Therapeutic peptides and proteins: formulation, processing, and delivery systems. 2nd ed. Boca Raton, Fla.: Taylor & Francis Group; 2006; Stevenson. Curr Pharm Biotechnol 2009, 10:122-137; Wu and Chi, U.S. Pat. No. 9,387,257; Wu, et al., U.S. Pat. No. 8,415,453; Faure, et al., U.S. Pat. No. 8,013,116; Faure, et al., U.S. Pat. No. 9,273,111; Eggink and Hoober, U.S. Pat. No. 7,811,995; Eggink and Hoober, U.S. Pat. No. 8,496,942; Morgan and Pandha. US 2012/0177672 A1; Broersma, et al., U.S. Pat. No. 5,681,925), each of which is herein incorporated by reference in its entirety.

In one embodiment, this domain comprises the Toll Like Receptor (TLR) modulatory sequence (see e.g. Tsung, et al. Shock 2007, 27:364-369). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative TLR-related trifunctional peptides: TLR/TRIOPEP DA32 (DIVKLTVYDCIRRRRRRRRRPLGEEMRDRARAHVDALRTHLA) (SEQ ID NO. 37) and TLR/TRIOPEP DE32 (DIVKLTVYDCIRRRRRRRRRPYLDDFQKKWQEEMELYRQKVE) (SEQ ID NO. 38). In one embodiment, methionine residues of TLR/TRIOPEP peptides are modified.

In certain embodiments, the capability of the TLR-related peptides and compounds of the present invention including but not limiting to those described above, to inhibit TLR can be used to treat and/or prevent TLR-related diseases and conditions including but not limiting to, sepsis and other infectious diseases, and any other disorder where TLR receptors are involved.

In certain embodiments, the capability of the TLR-related peptides and compounds of the present invention including but not limiting to those described above, to colocalize with TLR can be used to visualize (image) this receptor and evaluate its expression in the areas of interest. In one embodiment, for this purpose, an imaging probe (e.g. [⁶⁴Cu]) can be conjugated to the TLR/TRIOPEP sequences In one embodiment, imaging (visualization) of TLR levels using PET and/or other imaging techniques can be used to diagnose TLR-related diseases and conditions as well as to monitor novel therapies for these diseases and conditions.

In one embodiment, the domain A of the peptides and compositions of the invention comprises the Atrial Natriuretic Peptide (ANP) receptor (ANPR)-modulatory sequence (see e.g. Ladetzki-Baehs, et al. Endocrinology 2007, 148:332-336). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in its entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative ANPR-related trifunctional peptides: ANPR/TRIOPEP SA50 (SLRRSSCFGGRMDRIGAQSGLGCNSFRYPLGEEMRDRARAHVDALRTHLA) (SEQ ID NO. 39) and ANPR/TRIOPEP SE50 (SLRRSSCFGGRMDRIGAQSGLGCNSFRYPYLDDFQKKWQEEMELYRQKVE) (SEQ ID NO. 40). In one embodiment, methionine residues of ANPR/TRIOPEP peptides are modified.

In certain embodiments, the capability of the ANPR-related peptides and compounds of the present invention including but not limiting to those described above, to inhibit ANPRs can be used to treat and/or prevent ANPR-related diseases and conditions including but not limiting to, cardiovascular and inflammatory diseases, and any other disorder where ANP receptors are involved.

In certain embodiments, the capability of the ANPR-related peptides and compounds of the present invention including but not limiting to those described above, to colocalize with ANPR can be used to visualize (image) this receptor and evaluate its expression in the areas of interest. In one embodiment, for this purpose, an imaging probe (e.g. [⁶⁴Cu]) can be conjugated to the ANPR/TRIOPEP sequences In one embodiment, imaging (visualization) of ANPR levels using PET and/or other imaging techniques can be used to diagnose ANPR-related diseases and conditions as well as to monitor novel therapies for these diseases and conditions.

In certain embodiments, other therapeutic agents including but not limiting to, to those described in Page and Takimoto. Principles of chemotherapy. In: Pazdur R, Wagman L D, Camphausen K A, editors. Cancer Management: A Multidisciplinary Approach. 11th ed. Manhasset, N.Y.: Cmp United Business Media; 2009. p. 21-37; Sipsas, et al., Therapy of Mucormycosis, J Fungi (Basel) 2018, 4; Lin, et al. Chem Commun (Camb) 2013, 49:4968-4970; and in Turner, et al. Peptide Conjugates of Oligonucleotide Analogs and siRNA for Gene Expression Modulation. In: Langel U, ed, editor. Handbook of Cell-Penetrating Peptides. 2nd edition ed. Boca Raton: CRC Press; 2007. p. 313-328 and disclosed in Schiffman and Altman, U.S. Pat. No. 4,427,660; Castaigne, et al., U.S. Pat. No. 9,161,988; Castaigne, et al., U.S. Pat. No. 8,921,314; and in Castaigne, et al., U.S. Pat. No. 9,173,891, each of which is herein incorporated by reference in its entirety (see also TABLE 2) can comprise the domain A of the peptides and compositions of the present invention.

III. Lipoproteins and rHDLS.

Lipoproteins, including circulating lipoproteins in blood plasma, are natural complexes that contain both proteins (apolipoproteins, apo) and lipids bound to the proteins, which allow water-insoluble molecules such as fats to move through the water inside and outside cells. Lipoproteins serve to emulsify the lipid molecules. Examples include the plasma lipoprotein particles classified under high-density lipoproteins (HDL), which enable cholesterol and other hydrophobic lipid molecules to be carried in the bloodstream. In particular, HDL transport cholesterol and other water insoluble or poorly soluble lipids from the peripheral tissues to the liver.

The use of HDLs as delivery vehicles was proposed however in order to properly function in vivo for delivery of drugs or imaging agents to sites of interest, HDLs should mimic native lipoproteins as close as possible. In a human body, HDL exists in two forms: nascent or discoidal HDL and spherical HDL. The use of isolated plasma lipoproteins, including isolated HDLs, as delivery vehicles is impractical.

However in vitro, long half-life lipoprotein particles that mimic native HDL (as synthetic sHDL or recombinant HDL, rHDL) can be readily reconstituted (synthesized) from lipid formulations and apolipoproteins (apo) resulting in, for example, sub 30 nm-sized particles of discoidal or spherical morphology. Morphology of rHDLs is determined by the composition of lipid and apo mixtures and preparation procedures.

Many types of rHDLs were evaluated both clinically and experimentally as a delivery system for administering hydrophobic agents and for mitigating the toxic effects associated with administration of imaging probes such as Gd-containing contrast agents (GBCAs) for magnetic resonance imaging (MRI).

As delivery vehicles, rHDL have several competitive advantages as compared with other delivery platforms: 1) apo A-I, a major HDL protein, is used for rHDL preparation as it's recombinant or synthesized peptide/protein represents an endogenous protein that does not trigger immunoreactions; 2) apo A-I's small size allows rHDL to pass through blood vessel walls, enter and then accumulate in the places of interest, including for treatment and/or detection, such as tumor sites, areas of disease, such as liver tissue, etc., or atherosclerotic plaques; 3) rHDL's small particle size also allows for intravenous, intramuscular and subcutaneous applications; 4) rHDL's naturally long half-life extends the half-life of incorporated drugs and/or imaging agents in a bloodstream; and 5) a variety of drugs and imaging agents can be incorporated into this platform.

However, in order to properly function in vivo and as a result, to realize all the advantages mentioned above, rHDL should mimic native lipoproteins including but not limited to HDL as close as possible. This is a complicated task because two functions, assistance in the self-assembly of rHDL and therapeutic and/or imaging action in vivo, have to be executed by at least, two separate rHDL ingredients such as human apolipoprotein and therapeutic agent and/or imaging probe. In addition, in contrast to, for example, native HDL that are normally target the liver, rHDL have to be able to target other sites of interest such as, for example, macrophages which results in the need of targeting moieties thus adding the third function of rHDL ingredients—targeting. This hampers wider use of rHDL by difficulties in industrializing the manufacture of rHDL, along with rHDL′ lack of stability and reproducibility. In addition, the use of native or recombinant human apolipoproteins significantly complicates development of the commercial product, drastically increases its cost and possesses potential clinical and regulatory pitfalls.

An alternative, fully synthetic lipopeptide system for targeted treatment and/or imaging that closely mimics native lipoproteins and exhibits the advantageous properties of rHDL as well as superior stability, uniformity, ease of use, and reproducibility of preparation is needed for administration and targeted delivery of therapeutic agents (e.g. anti-cancer and anti-sepsis agents, other anti-inflammatory drugs) and/or imaging probes. The invention provides such a system and a method of using the system (e.g., for delivery of anti-cancer, anti-arthritic, anti-sepsis, anti-angiogenic and other therapeutic agents and/or imaging probes to a subject). These and other objects and advantages of the invention, as well as additional inventive features, will be apparent from the description of the invention provided herein.

Additional contemplative advantages of a lipoprotein delivery platform includes increasing activity due to specific targeting, sequestration of the drug at the target site, protection of the drug from rapid metabolism, amplified therapeutic effect due to packaging of numerous drug molecules in each particle, and decreased toxicity due to altered pharmacokinetics. Due to the naturally long half-life of native discoidal and spherical HDL in normal subjects being 12-20 hrs and 3-5 days, respectively, rHDL represent a promising versatile delivery platform in particular for therapeutic peptides that have a bloodstream half-life of minutes.

For example, it would be desirable to combine in one molecule therapeutic (and/or diagnostic), particle forming and targeting functions. The invention addresses these needs, among others, and provides such a system/molecule and a method of using the system (e.g., for delivery of anti-cancer, anti-arthritic, anti-sepsis, anti-angiogenic, anti-inflammatory and other therapeutic agents and/or imaging probes to a subject). These and other objects and advantages of the invention, as well as additional inventive features, will be apparent from the description of the invention provided herein.

IV. Trifunctional Peptides In rHDL Formulations.

A. TREM-1-Related Trifunctional Peptides: TREM-1 Signaling Pathway and its Blockade.

TREM-1 is expressed on the majority of innate immune cells and to a lesser extent on parenchymal cells. Upon activation, TREM-1 can directly amplify an inflammatory response. Although it was initially demonstrated that TREM-1 was predominantly associated with infectious diseases, recent evidences demonstrate that TREM-1 receptor and its signaling pathways contribute to the pathology of non-infectious acute and chronic inflammatory diseases, including but not limiting to, rheumatoid arthritis, atherosclerosis, ischemia reperfusion-induced tissue injury, colitis, fibrosis, neurodegenerative diseases, liver diseases, retinopathies, and cancer (see e.g., Tammaro, et al. Pharmacol Ther 2017, 177:81-95; Saadipour. Neurotox Res 2017, 32:14-16; Sigalov. Adv Protein Chem Struct Biol 2018, 111:61-9; Sigalov. U.S. Pat. No. 8,513,185; Sigalov. U.S. Pat. No. 9,981,004; Rojas, et al. Biochim Biophys Acta 2018, 1864: 2761-2768, and Kuai, et al. US 2008/0247955, each of which is herein incorporated by reference in its entirety).

In some preferred embodiments, TREM-1-related peptides and associated compositions of the present invention have a domain A conjugated to a domain B. See, FIG. 1. Domain A comprises a TREM-1 modulatory peptide sequence designed using a known model of cell receptor signaling, the Signaling Chain HOmoOLigomerization model, capable of modulating TREM-1 receptor expressed on myeloid cells (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. U.S. Pat. No. 8,513,185; and Sigalov. U.S. Pat. No. 9,981,004), all of which are herein incorporated by reference in their entirety. In some preferred embodiments, peptides and compositions of the present invention comprise the TREM-1 modulatory peptide sequences designed using a well-known in the art novel model of cell receptor signaling, the Signaling Chain HOmoOLigomerization model.

In some preferred embodiments, peptides and compositions of this class further comprise the domain B comprising at least one modified or unmodified amphipathic alpha helical peptide fragment, such as a apo A-I and/or A-II peptide fragment, to form upon interaction with lipid and/or lipid mixtures. In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In one embodiment, preferred peptides and compositions of the invention further comprise at least one modified or unmodified amphipathic apo A-I and/or A-II peptide fragment capable upon interaction with lipid and/or lipid mixtures, to form synthetic lipopeptide particles (SLP) structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Sigalov. US 20130039948; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768). The inclusion of an amphipathic apo A-I sequences in the peptides and compositions of the invention further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

FIG. 1 presents an exemplary schematic representation of one embodiment of a trifunctional peptide of the present invention comprising amino acid domains A and B where amino acid domain A represents a therapeutic peptide sequence with or without an attached drug compound and/or imaging probe that functions to treat, prevent and/or detect a disease or condition, whereas amino acid domain B represents an amphipathic alpha helical peptide sequence, with or without an additional targeting peptide sequence, and functions to 1) assist in the self-assembly of synthetic lipoprotein/lipopeptide nanoparticles (SLP) upon interaction with lipids or lipid mixtures in vitro, for use in transporting these trifunctional peptides as lipoprotien nanoparticles to sites of interest in vitro or in vivo and/or 2) form long half-life lipopeptide/lipoprotein particles upon interaction with endogenous lipoproteins for transporting these trifunctional peptides to the sites of interest. Endogenous lipoproteins may be lipoproteins added to or found in cell cultures, or lipoproteins in a mammalian body.

In certain embodiments, FIG. 2 shows the structures of representative TREM-1-related trifunctional peptides, TREM-1/TRIOPEP GE31 (GFLSKSLVFPYLDDFQKKWQEEM(O)ELYRQKVE, M(O), methionine sulfoxide) (SEQ ID NO. 4) and TREM-1/TRIOPEP GA31 (GFLSKSLVFPLGEEM(O)RDRARAHVDALRTHLA, M(O), methionine sulfoxide) (SEQ ID NO. 3). In one embodiment, methionine residues of the peptides TREM-1/TRIOPEP GE31 (GFLSKSLVFPYLDDFQKKWQEEMELYRQKVE) (SEQ ID NO. 2) and TREM-1/TRIOPEP GA31 (GFLSKSLVFPLGEEMRDRARAHVDALRTHLA) (SEQ ID NO. 1) are unmodified. GFLSKSLVF, is found within isoform 1 of human TREM-1 (UniProtKB—Q9NP99 (TREM1_HUMAN), and in human TREM-1 isoform CRA_a (UniProtKB—Q38L15 (Q38L15_HUMAN), both downloaded Oct. 24, 2018)).

Q9NP99|TREM1_HUMAN Isoform 1 Triggering receptor expressed on myeloid cells 1, Homo sapiens:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKSTADVSTPDSEINLTNVTDIIR
VPVFNIVILLAGGFLSKSLVFSVLFAVTLRSFVP

Q38L15|Q38L15_HUMAN Triggering receptor expressed on myeloid cells 1, Homo sapiens isoform CRA_a:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKSTADVSTPDSEINLTNVTDIIR
VPVFNIVILLAGGFLSKSLVFSVLFAVTLRSFVP

GFLSKSLVF, is not found within human TREM-1 isoforms 2 or 3.
Q9NP99-2|TREM1_HUMAN Isoform 2 of Triggering receptor expressed on myeloid cells 1, Homo sapiens:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFRCSTLSFSWLVDS

Q9NP99-3|TREM1_HUMAN Isoform 3 of Triggering receptor expressed on myeloid cells 1, Homo sapiens:

MRKTRLWGLLWMLFVSELRAATKLTEEKYELKEGQTLDVKCDYTLEKFAS
SQKAWQIIRDGEMPKTLACTERPSKNSHPVQVGRIILEDYHDHGLLRVRM
VNLQVEDSGLYQCVIYQPPKEPHMLFDRIRLVVTKGFSGTPGSNENSTQN
VYKIPPTTTKALCPLYTSPRTVTQAPPKST.

FIG. 2 presents schematic representations of embodiments of a TREM-1-related trifunctional peptide (TREM-1/TRIOPEP). GE31 (GFLSKSLVFPYLDDFQKKWQEEM(O)ELYRQKVE, M(O), methionine sulfoxide) comprising amino acid domain A and B (GFLSKSLVFPYLDDFQKKWQEEM(O)ELYRQKVE) where domain A represents a 9 amino acids-long human TREM-1 inhibitory therapeutic SCHOOL peptide sequence and functions to treat and/or prevent a TREM-1-related disease or condition, whereas domain B represents a 22 amino acids-long human apolipoprotein A-I helix 4 peptide sequence with a sulfoxidized methionine residue and functions to assist in the self-assembly of synthetic lipopeptide particles (SLP) in vitro for targeting the particles to myeloid cells (e.g. macrophages). GA31 (GFLSKSLVFPLGEEM(O)RDRARAHVDALRTHLA, M(O), methionine sulfoxide). Abbreviations: apo, apolipoprotein; SCHOOL, signaling chain homooligomerization; TREM-1, triggering receptor expressed on myeloid cells-1.

In certain embodiments, other preferred TREM-1-related trifunctional peptides and compositions of this class comprise the domain A comprising the TREM-1 inhibitory peptide sequences LR12 and LP17 (described in Gibot, et al. Infect Immun 2006, 74:2823-2830; Gibot, et al. Shock 2009, 32:633-637; Gibot, et al. Eur J Immunol 2007, 37:456-466; Joffre, et al. J Am Coll Cardiol 2016, 68:2776-2793; Cuvier, et al. Br J Clin Pharmacol 2018, in press; Zhou, et al. Int Immunopharmacol 2013, 17:155-161; and disclosed in Faure, et al., U.S. Pat. No. 8,013,116; Faure, et al., U.S. Pat. No. 9,273,111; Gibot, et al., U.S. Pat. No. 9,657,081; Gibot and Derive, U.S. Pat. No. 9,815,883; and in Gibot and Derive, U.S. Pat. No. 9,255,136, each of which is herein incorporated by reference in it's entirety) while the domain B comprises at least one modified or unmodified amphipathic apo A-I and/or A-II peptide fragment to form upon interaction with lipid and/or lipid mixtures, SLP structures that can be spherical or discoidal. In some embodiments, resulting trifunctional peptide sequences may be radiolabeled and/or contain unmodified or modified methionine residues (TABLE 2) including but not limiting to, the following sequences:

LQEEDAGEYGCMPLGEEM(O)RDRARAHVDALRTHLA (M(O), methionine sulfoxide (SEQ ID NO 7), LQEEDAGEYGCMPYLDDFQKKWQEEM(O)ELYRQKVE (M(O), methionine sulfoxide (SEQ ID NO 8),
LQVTDSGLYRCVIYHPPPLGEEM(O)RDRARAHVDALRTHLA (M(O), methinone sulfoxide (SEQ ID NO 9), LQVTDSGLYRCVIYHPPPYLDDFQKKWQEEM(O)ELYRQKVE (M(O), methionine sulfoxide (SEQ ID NO 10).

SLP (rHDL) structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Sigalov. US 20130039948; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety). The inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In one embodiment, methionine residues of the peptides TREM-1/TRIOPEP GE31 (GFLSKSLVFPYLDDFQKKWQEEMELYRQKVE) (SEQ ID NO. 2) and TREM-1/TRIOPEP GA31 (GFLSKSLVFPLGEEMRDRARAHVDALRTHLA) (SEQ ID NO. 1) are unmodified. In one embodiment, interaction of TREM-1/TRIOPEP GA31 with lipids results in self-assembly of nanosized SLP of discoidal or spherical morphology (dSLP and sSLP, respectively) (see FIG. 3). FIG. 3 presents a schematic representation of one embodiment of a TREM-1-related trifunctional peptide (TREM-1/TRIOPEP) of the present invention comprising amino acid domains A and B. Depending on lipid mixture compositions added to the peptides, sub 50 nm-sized SLP particles of discoidal (TREM-1/TRIOPEP-dSLP) or spherical (TREM-1/TRIOPEP-sSLP) morphology are self-assembled upon binding of the trifunctional peptide to lipids. Abbreviations: apo, apolipoprotein; SCHOOL, signaling chain homooligomerization; TREM-1, triggering receptor expressed on myeloid cells-1.

In one embodiment, this provides targeted delivery of the SLP constituents including TREM-1/TRIOPEP to intraplaque macrophages in vivo (FIG. 4A). In one embodiment, this provides targeted delivery of the SLP constituents including TREM-1/TRIOPEP to tumor-associated macrophages (TAMs) in vivo (FIG. 4B).

FIG. 4A illustrates a hypothesized molecular mechanism of action of one embodiment of a trifunctional peptide (TRIOPEP) of the present invention comprising amino acid domains A and B where domain A represents a 9 amino acids-long TREM-1 inhibitory therapeutic peptide sequence and functions to treat and/or prevent a TREM-1-related disease or condition (example, for atherosclerosis), whereas domain B represents a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of atherosclerosis. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in intraplaque macrophages is accompanied by accumulation of TRIOPEP in these cells. In contrast, native HDL particles that contain only unmodified apolipoprotein molecules are not recognized by intraplaque macrophages and return to the circulation.
FIG. 4B illustrates a hypothesized molecular mechanism of action of one embodiment of a trifunctional peptide (TRIOPEP) of the present invention comprising amino acid domains A and B where domain A is a 9 amino acids-long TREM-1 inhibitory therapeutic peptide sequence and functions to treat and/or prevent a TREM-1-related disease or condition (example, for cancer), whereas domain B is a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of cancer. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in tumor-associated macrophages is accompanied by accumulation of TRIOPEP in these cells. In contrast, native HDL particles that contain only unmodified apolipoprotein molecules are not recognized by tumor-associated macrophages and return to the circulation.
FIG. 4C shows a symbol key used in FIGS. 4A-B.

While not being bound to any particular theory, it is believed that in one embodiment, this colocalization is accompanied by a specific disruption of intramembrane interactions between TREM-1 and DAP-12 by the TREM-1-related trifunctional peptide of the present invention (see FIG. 5), resulting in ligand-independent inhibition of TREM-1 upon ligand binding as described in Shen and Sigalov. Mol Pharm 2017, 14:4572-4582 and Shen and Sigalov. J Cell Mol Med 2017, 21:2524-2534, each of which is herein incorporated by reference in it's entirety

FIG. 5 illustrates one embodiment of a specific disruption of intramembrane interactions between TREM-1 and DAP-12 by the trifunctional peptide of the present invention comprising two amino acid domains A and B where domain A is a 9 amino acids-long TREM-1 inhibitory therapeutic peptide sequence, whereas domain B is a 22 amino acids-long apolipoprotein A-I helix 4 or 6 peptide sequence with a sulfoxidized methionine residue. While not being bound to any particular theory, it is believed that this disruption results in “pre-dissociation” of a receptor complex and upon ligand stimulation, leads to inhibition of TREM-1 and silencing the TREM-1 signaling pathway.

In one embodiment, FIG. 6 shows that the fluorescently labeled TREM-1/TRIOPEP peptide GE31 delivered to macrophages by the SLP particles of the present invention colocalizes with TREM-1 expressed on these cells (see also Rojas, et al. Biochim Biophys Acta 2018, 1864:2761-2768). In certain embodiments, the capability of the TREM-1-related trifunctional peptides and compounds of the present invention including but not limiting to, TREM-1/TRIOPEP GA31 and TREM-1/TRIOPEP GE31, to colocalize with TREM-1 can be used to visualize (image) this receptor and evaluate its expression in the areas of interest. In one embodiment, for this purpose, an imaging probe (e.g. [⁶⁴Cu], see TABLE 2) can be conjugated to the TREM-1/TRIOPEP sequences, GE31 (GFLSKSLVFPYLDDFQKKWQEEM(O)ELYRQKVE, M(O), methionine sulfoxide) (SEQ ID NO. 4) and GA31 (GFLSKSLVFPLGEEM(O)RDRARAHVDALRTHLA (M(O), methionine sulfoxide) (SEQ ID NO. 3). In one embodiment, imaging (visualization) of TREM-1 levels using PET and/or other imaging techniques can be used to diagnose glioblastoma multiforme (GBM) and/or to select and monitor novel GBM therapies (see e.g., Johnson, et al. Neuro Oncol 2017, 19:vi249 and James and Andreasson, WO 2017083682A1). In certain embodiments, imaging (visualization) of TREM-1 levels can be used to diagnose other TREM-1-related diseases and conditions as well as to monitor novel therapies for these diseases and conditions.

FIG. 6A-C shows images depicting colocalization of the sulfoxidized methionine residue-containing TREM-1-related trifunctional peptide (TREM-1/TRIOPEP) GE31 with TREM-1 in the cell membrane (FIG. 6A), TREM-1 immunohistochemistry staining (FIG. 6B) and a merged image (FIG. 6C).

As described herein (see FIG. 7A-B), sulfoxidation of methionine residues in the TREM-1/TRIOPEP peptides GE31 and GA31 results in increased macrophage endocytosis of the SLP containing an equimolar mixture of these peptides (designated as TREM-1/TRIOPEP), TREM-1/TRIOPEP-dSLP and TREM-1/TRIOPEP-sSLP. Shen and Sigalov. Mol Pharm 2017, 14:4572-4582 and Shen and Sigalov. J Cell Mol Med 2017, 21:2524-2534, all of which are herein incorporated in their entirety.

FIG. 7A-B presents the exemplary data showing the endocytosis of synthetic lipopeptide particles (SLP) of discoidal (dSLP) and spherical (sSLP) morphology that contain an equimolar mixture of the TREM-1-related trifunctional peptides (TREM-1/TRIOPEP) GA 31 and GE 31 (TREM-1/TRIOPEP-dSLP and TREM-1/TRIOPEP-sSLP, respectively). (FIG. 7A) The post 4 h incubation in vitro macrophage uptake of TREM-1/TRIOPEP-dSLP and TREM-1/TRIOPEP-sSLP with the unmodified (patterned bars) or sulfoxidized TREM-1/TRIOPEP methionine residues (black bars). ***, P=0.0001 to 0.001 (sulfoxidized vs. unmodified methionine residues). (FIG. 7B) the in vitro macrophage uptake of TREM-1/TRIOPEP-dSLP and TREM-1/TRIOPEP-sSLP with the sulfoxidized TREM-1/TRIOPEP methionine residues post 4 (white bars), 12 (patterned bars), and 24 h (black bars) incubation. ***, P=0.0001 to 0.001 as compared with 4 h incubation time.

In certain embodiments, FIGS. 8 and 10 demonstrate that TREM-1/TRIOPEP in free and SLP-bound forms inhibits TREM-1 function as shown by reduction of TREM-1-mediated release of pro-inflammatory cytokines both in vitro (FIG. 8) and in vivo (FIG. 10). While not being bound to any particular theory, it is believed that this indicates that similarly to TREM-1-inhibitory peptide GF9 (see e.g., Sigalov. Int Immunopharmacol 2014, 21:208-219; Shen and Sigalov. Mol Pharm 2017, 14:4572-4582; and Shen and Sigalov. J Cell Mol Med 2017, 21:2524-2534, each of which is herein incorporated by reference in it's entirety), TREM-1-related trifunctional peptides can reach their site of action from both outside (free TREM-1/TRIOPEP) and inside (SLP-bound TREM-1/TRIOPEP) the cell. It is also believed that upon administration, free TREM-1/TRIOPEP may form LP in vivo and/or interact with native lipoproteins, resulting in formation of HDL-mimicking LP. In one embodiment, these LP may further target the cells of interest delivering their content to the areas of interest in a body.

FIG. 8 presents the exemplary data showing suppression of tumor necrosis factor-alpha (TNF-alpha), interleukin (IL)-6 and IL-1beta production by lipopolysaccharide (LPS)-stimulated macrophages incubated for 24 h at 37° C. with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. ***, P=0.0001 to 0.001 as compared with medium-treated LPS-challenged macrophages.
FIG. 10 presents the exemplary data showing suppression of tumor necrosis factor-alpha (TNF-alpha), interleukin (IL)-6 and IL-1beta production in mice at 90 min post lipopolysaccharide (LPS) challenge treated 1 h before LPS challenge with phosphate-buffer saline (PBS), dexamethasone (DEX), control peptide and with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. Control peptide represents an equimolar mixture of two peptides, each of them comprising two amino acid domains A and B where domain A represents a non-functional 9 amino acids-long sequence of the TREM-1 inhibitory therapeutic peptide sequence wherein, Lys₅ is substituted with Ala₅, whereas domain B is a sulfoxidized methionine residue-containing 22 amino acids-long apolipoprotein A-I helix 4 or 6 peptide sequence, respectively. *, P=0.01 to 0.05 as compared with animals treated with 5 mg/kg TRIOPEP in free form; ***, P=0.0001 to 0.001 as compared with PBS-treated animals.

While not being bound to any particular theory, it is believed that increased uptake described herein, is mediated by macrophage scavenger receptors (SR) including, but not limiting to, SR-A and SR-B1 (see FIG. 9A-C). While not being bound to any particular theory, it is believed that in one embodiment, this colocalization is accompanied by a specific disruption of intramembrane interactions between TREM-1 and DAP-12 by the TREM-1-related trifunctional peptide of the present invention (see FIG. 9A), resulting in ligand-independent inhibition of TREM-1 upon ligand binding as described in Shen and Sigalov. Mol Pharm 2017, 14:4572-4582 and Shen and Sigalov. J Cell Mol Med 2017, 21:2524-2534, each of which is herein incorporated by reference in it's entirety.

FIG. 9A1-A2-C shows schematic representations of activation of the TREM-1/DAP12 receptor complex expressed on macrophages and presents the exemplary data showing that scavenger receptors SR-A and SR-B1 mediate the macrophage endocytosis of GF9-sSLP (GF9-HDL) and GA/E31-HDL (TREM-1/TRIOPEP-sSLP). (FIG. 9A) Schematic representation of TREM-1 signaling and the SCHOOL mechanism of TREM-1 blockade. (FIG. 9A1, left panel) Activation of the TREM-1/DAP12 receptor complex expressed on macrophages leads to phosphorylation of the DAP12 cytoplasmic signaling domain and subsequent downstream inflammatory cytokine response (left panel). SR-mediated endocytosis of sSLP-bound GF9, GA31 and GE31 peptide inhibitors by macrophages results in the release of GF9 or GA31 and GE31 into the cytoplasm, which self-penetrate into the cell membrane and block intramembrane interactions between TREM-1 and DAP12, thereby preventing DAP12 phosphorylation and downstream signaling cascade (FIG. 9A1, right panel).
FIG. 9A2, left panel shows schematic representations of activation of the TREM-1/DAP12 receptor complex expressed on Kupffer cells leads to phosphorylation of the DAP12 cytoplasmic signaling domain, subsequent SYK recruitment, and the downstream inflammatory cytokine response. (FIG. 9A2, right panel) SR-mediated endocytosis of HDL-bound GF9 peptide inhibitors by Kupffer cells results in the release of GF9 (GA31 or GE31) into the cytoplasm; GF9 self-penetrates the cell membrane and blocks intramembrane interactions between TREM-1 and DAP12, thereby preventing DAP12 phosphorylation and the downstream signaling cascade.
FIG. 9B-9C Macrophage endocytosis of GF9-sSLP (GF9-HDL) and GA/E31-HDL (TREM-1/TRIOPEP-sSLP) in vitro is SR-mediated in a time-dependent manner and is largely driven by SR-A (FIG. 9B, FIG. 9C). As described in the Materials and Methods, J774 macrophages were cultured at 37° C. overnight with medium. Prior to uptake of GF9-HDL and GA/E31-HDL, cells were treated for 1 hour at 37° C. with 40 μM cytochalasin D and either (FIG. 9B) 400 μg/mL fucoidan or (FIG. 9C) 10 μM BLT-1, as indicated. Cells were then incubated for either 4 hours or 22 hours with medium containing 2 μM rhodamine B (rho B)-labeled GF9-sSLP (gray bars) or TREM-1/TRIOPEP-sSLP (black bars), respectively. Cells were lysed, and rho B fluorescence intensities of lysates were measured and normalized to the protein content. Results are expressed as mean SEM (n=3); *P≤0.05; **P≤0.01; ****P≤0.0001 versus uptake of GF9-HDL and GA/E31-HDL in the absence of inhibitor. Abbreviations: D, DAP12; DAP12, DNAX activation protein of 12 kDa; K, Kupffer cell; RFU, relative fluorescence units; SCHOOL, signaling chain homo-oligomerization.

In certain embodiments, FIGS. 11A-B-14A-C demonstrate that TREM-1/TRIOPEP in free and SLP-bound forms inhibits tumor growth, reduces infiltration of macrophages into the tumor in mouse models of NSCLC and PC and is well-tolerated by cancer mice during the treatment period (see also Shen and Sigalov. Mol Pharm 2017, 14:4572-4582).

FIG. 11A-B presents the exemplary data showing inhibition of tumor growth in the human non-small cell lung cancer H292 (FIG. 11A) and A549 (FIG. 11B) xenograft mice treated with an equimolar mixture of the sulfoxidized methionine residue-containing TREM-1-related trifunctional peptides (TREM-1/TRIOPEP) GA 31 and GE 31 in free form. PTX, paclitaxel. ****, P≤0.0001 as compared with vehicle-treated animals.
FIG. 12A-B presents the exemplary data showing inhibition of tumor growth in the human non-small cell lung cancer H292 (FIG. 12A) and A549 (FIG. 12B) xenograft mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.
FIG. 13 presents the exemplary data showing average tumor weights in the A549 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.
FIG. 14A-C presents the exemplary data showing inhibition of tumor growth (FIG. 14A) and TREM-1 blockade-mediated suppression of intratumoral macrophage infiltration (FIG. 14B, FIG. 14C) in the human pancreatic cancer BxPC-3 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. (B) F4/80 staining. Results are expressed as the mean±SEM (n=4 mice per group). *, p<0.05; **, p<0.01, ****, p<0.0001 (versus vehicle). (FIG. 14C) Representative F4/80 images from BxPC-3-bearing mice treated using different free and sSLP-bound SCHOOL TREM-1 inhibitory GF9 sequences including TREM-1/TRIOPEP-sSLP. Scale bar=200 μm.

In embodiment, FIG. 15 demonstrates that TREM-1/TRIOPEP in free and SLP-bound forms significantly prolongs survival in mice with lipopolysaccharide (LPS)-induced septic shock.

FIG. 15A-B presents the exemplary data showing improved survival of lipopolysaccharide (LPS)-challenged mice treated with an equimolar mixture of the sulfoxidized methionine residue-containing TREM-1-related trifunctional peptides (TREM-1/TRIOPEP) GA 31 and GE 31 in free form (FIG. 15A) or incorporated into synthetic lipopeptide particles (SLP) particles of discoidal (TREM-1/TRIOPEP-dSLP) and spherical (TREM-1/TRIOPEP-sSLP) morphology.
FIG. 15B. **, P=0.001 to 0.01 as compared with vehicle-treated animals.

In one embodiment, FIG. 16 shows that TREM-1/TRIOPEP is non-toxic in healthy mice at least up to 400 mg/kg.

FIG. 16 presents exemplary data showing average weights of healthy C57BL/6 mice treated with increasing concentrations of an equimolar mixture of the sulfoxidized methionine residue-containing TREM-1-related trifunctional peptides (TREM-1/TRIOPEP) GA 31 and GE 31 in free form.

In one embodiment, FIG. 17 demonstrates that TREM-1/TRIOPEP in free and SLP-bound form ameliorates arthritis in mice with collagen-induced arthritis (CIA) and is well-tolerated by arthritic mice during the treatment period of 2 weeks (see Shen and Sigalov. J Cell Mol Med 2017, 21:2524-2534).

FIG. 17A-B presents the exemplary data showing average clinical arthritis score (FIG. 17A) and mean body weight (BW) changes (FIG. 17B) calculated as a percentage of the difference between beginning (day 24) and final (day 38) BWs of the collagen-induced arthritis (CIA) mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. DEX, dexamethasone. *, p<0.05, **, p<0.01; ***, p<0.001 as compared with vehicle-treated or naive animals.

In certain embodiments, FIG. 18 demonstrates that TREM-1/TRIOPEP-sSLP prevents pathological RNV in mice with oxygen-induced retinopathy and is well-tolerated by these mice during the treatment period (see Rojas, et al. Biochim Biophys Acta 2018, 1864:2761-2768). FIG. 18A-D presents the exemplary data showing reduction of pathological retinal neovascularization area (FIG. 18A), avascular area (FIG. 18B) and retinal TREM-1 (FIG. 18C) and M-CSF/CSF-1 (FIG. 18D) expression in the retina of the mice with oxygen-induced retinopathy (OIR) treated with an equimolar mixture of the sulfoxidized methionine residue-containing TREM-1-related trifunctional peptides (TREM-1/TRIOPEP) GA 31 and GE 31 incorporated into synthetic lipopeptide particles (TREM-1/TRIOPEP-SLP) particles of spherical morphology (TREM-1/TRIOPEP-sSLP). ***, p<0.001 as compared with vehicle-treated animals.

As described in Stukas, et al. J Am Heart Assoc 2014, 3:e001156, systemically administered human apo A-I accumulates in murine brain. It is also known that transcytosis of HDL in brain microvascular endothelial cells is mediated by SRBI (see Fung, et al. Front Physiol 2017, 8:841). However, until tested, it was not known that a self-assembled SLP of the present invention comprising a trifunctional peptide was capable of crossing the BBB.

In certain embodiments, FIG. 19 shows that the self-assembled SLP of the present invention may cross the BBB, BRB and BTB, thus delivering their constituents including but not limiting to, TREM-1/TRIOPEP to the areas of interest in the brain, retina and tumor. While not being bound to any particular theory, it is believed that the brain-, retina-, and tumor-penetrating capabilities of these SLP can be mediated by interaction of SRBI with the domain B amino acid sequences that correspond to the sequences of human apo A-I helices 4 and/or 6 (see e.g. Liu, et al. J Biol Chem 2002, 277:21576-21584, each of which is herein incorporated by reference in it's entirety).

In certain embodiments, these capabilities of the peptides and compositions of the present invention can be used to diagnose, treat and/or prevent cancers (including brain cancer), diabetic retinopathy and retinopathy of prematurity, neurodegenerative diseases including Alzheimer's, Parkinson's and Huntington's diseases and other diseases and conditions where delivery of the peptides and compositions of the invention to the brain, retina and/or tumor is needed.

FIG. 19 presents exemplary data showing penetration of the blood-brain barrier (BBB) and blood-retinal barrier (BRB) by systemically (intraperitoneally) administered rhodamine B-labeled spherical self-assembled particles (sSLP) that contain Gd-containing contrast agent (Gd-sSLP) for magnetic resonance imaging (MRI), TREM-1 inhibitory peptide GF9 (GF9-sSLP) or an equimolar mixture of the sulfoxidized methionine residue-containing TREM-1-related trifunctional peptides GA 31 and GE 31 (TREM-1/TRIOPEP-sSLP).

Mouse model of ALD mimics the early phase of the human disease, yet mRNA levels of early fibrosis markers Pro-Colla and a-SMA were significantly increased in alcohol-fed mice compared to PF controls in the whole-liver samples (FIG. 20A-B). Induction of these makers was remarkably attenuated in the vehicle-treated group and, importantly, further decreased by the TREM-1 inhibitory formulations used (FIG. 20A-B).

FIG. 20A-B presents exemplary data showing TREM-1/TRIOPEP-sSLP suppresses the expression of fibrinogenesis marker molecules, FIG. 20A Pro-Collagen 1α and FIG. 20B α-Smooth Muscle Actin, at the RNA level, as measured in whole-liver lysates of mice with (alcohol-fed) and without (pair-fed) alcoholic liver disease (ALD).
* indicates significance level compared to the non-treated pair-fed (PF) group; #indicates significance level compared to the non-treated alcohol-fed group. o indicates significance level compared to the vehicle-treated alcohol-fed group. The significant levels are as follows: *, 0.05≥P≥0.01; **, 0.01≥P≥0.001; ***, 0.001≥P≥0.0001; ****, P≤0.0001.

TREM-1 inhibitor effects were evaluated on hepatocyte damage and steatosis in liver. Serum ALT levels obtained during week 5 of the alcohol feeding showed significant increases in alcohol-fed mice compared to PF controls. This ALT increase was attenuated in both TREM-1 inhibitor-treated groups, indicating attenuation of liver injury (FIG. 4A).

Surprisingly, vehicle treatment (HDL) also showed a similar protective effect (FIG. 4A).

Consistent with steatosis, we found a significant increase in Oil Red O staining in livers of alcohol-fed mice compared to PF controls (FIG. 4C). Oil Red O (FIG. 4B-D) and H&£ (FIG. 4D) staining revealed attenuation of steatosis in the alcohol-fed TREM-1 inhibitor-treated mice compared to both untreated and vehicle (HDL)-treated alcohol-fed groups (FIG. 4B-D).

FIG. 21A-D presents exemplary data showing that TREM-1/TRIOPEP-sSLP suppresses the production of alanine aminotransferase (ALT) in mice with alcoholic liver disease (ALD), as measured in serum of mice with (alcohol-fed) and without (pair-fed) ALD, in addition to improving indicators of liver disease and inflammation. * indicates significance level compared to the alcohol-fed group treated with vehicle-synthetic lipopeptide particles of spherical morphology that contained an equimolar mixture of PE22 and PA22 (sSLP) but no TREM-1 inhibitory peptide GF9. #indicates significance level compared to the non-treated alcohol-fed group. Liver damage after 5 weeks of alcohol feeding and effect of TREM-1 pathway inhibition in a mouse model of ALD. sSLP, 5 mg/kg treatment of TREM-1 peptide vs. TREM-1/TRIOPEP-sSLP. Cheek blood and livers were harvested at death. (FIG. 21A) Serum ALT levels were measured using a kinetic method. Exemplary data showing TREM-1/TRIOPEP-sSLP suppresses alanine aminotransferase in serum of alcohol fed mice over TREM-1 peptide alone. (FIG. 21B-D) Liver sections were stained with (B,C) Oil Red O and (FIG. 21D) H&E staining, and the lipid content was analyzed by ImageJ (FIG. 21B). * indicates significance level compared to the nontreated PF group; * indicates significance level compared to the nontreated alcohol-fed group; ⁰ indicates significance level compared to the vehicle-treated alcohol-fed group. The numbers of the symbols sign the significant levels as the following: **^{o}P≤0.05; ^{##/oo}P≤0.01; *″^{/##}P≤0.001; ****P<0.0001. ***, 0.001≥P≥0.0001; ##, 0.01≥P≥0.001.

B. TCR-Related Trifunctional Peptides

The T-cell receptor (TCR)-CD3 complex plays a role in T-cell differentiation, in protecting the organism from infectious agents, and in the function of T-cells. The TCR is a complex of a heterodimer of TCRa and TCRb chains, which are responsible for antigen recognition and interaction with the major histocompatibility complex (MHC) molecules of antigen-presenting cells, and CD3d, CD3g, CD3e and CD3z chains, which are responsible for transmembrane signal transduction (see e.g., Manolios, et al. Cell Adh Migr 2010, 4:273-283; Sigalov. Adv Protein Chem Struct Biol 2018, 111:61-9; Sigalov. US 20130039948; Manolios. U.S. Pat. No. 6,057,294; Manolios. U.S. Pat. No. 7,192,928; Manolios. US 20100267651; and Manolios, et al. US 20120077732, each of which is herein incorporated by reference in it's entirety).

The preferred TCR-related peptides and compositions of this class comprise the domain A comprising the TCR modulatory peptide sequences designed using a well-known in the art novel model of cell receptor signaling, the Signaling Chain HOmoOLigomerization model, capable of modulating TCR (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. PLoS Pathog 2009, 5:e1000404; Shen and Sigalov. Sci Rep 2016, 6:28672; Sigalov. US 20130039948, each of which is herein incorporated by reference in it's entirety). The preferred peptides and compositions of this class further comprise the domain B comprising at least one modified or unmodified amphipathic alpha helical peptide fragment. As described above, the inclusion of an amphipathic amino acid sequences aids the assistance in the ability to interact with native lipoproteins in a bloodstream in vivo and to form naturally long half-life lipopeptide/lipoprotein particles LP. It further aids the ability to provide targeted delivery to the sites of interest. It further aids the ability to cross the BBB, BRB and BTB.

The preferred TCR-related peptides and compositions of this class comprise the domain A comprising the TCR modulatory peptide sequences designed using a well-known in the art novel model of cell receptor signaling, the Signaling Chain HOmoOLigomerization model, capable of modulating TCR (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. PLoS Pathog 2009, 5:e1000404; Shen and Sigalov. Sci Rep 2016, 6:28672; Sigalov. US 20130039948, each of which is herein incorporated by reference in it's entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative TCR-related trifunctional peptides: TCR/TRIOPEP MA32 (MWKTPTLKYFPLGEEMRDRARAHVDALRTHLA) (SEQ ID NO. 11), TCR/TRIOPEP ME32 (MWKTPTLKYFPYLDDFQKKWQEEMELYRQKVE) (SEQ ID NO. 12), TCR/TRIOPEP GA36 (GARSMTLTVQARQLPLGEEMRDRARAHVDALRTHLA) (SEQ ID NO 13), TCR/TRIOPEP GE36 (GARSMTLTVQARQLPYLDDFQKKWQEEMELYRQKVE) (SEQ ID NO 14), TCR/TRIOPEP GA32 (GVLRLLLFKLPLGEEMRDRARAHVDALRTHLA) (SEQ ID NO 15), and TCR/TRIOPEP GE32 (GVLRLLLFKLPYLDDFQKKWQEEMELYRQKVE) (SEQ ID NO 16). While not being bound to any particular theory, it is believed that in one embodiment, these peptides colocalize with TCR in the cell membrane and selectively disrupt intramembrane interactions of TCRa chain with the CD3ed heterodimer and CD3zz homodimer, resulting to specific ligand-independent inhibition of TCR upon antigen stimulation (see e.g. Sigalov. Self Nonself 2010, 1:4-39.; Sigalov. Self Nonself 2010, 1:192-224; and Sigalov. Adv Protein Chem Struct Biol 2018, 111:61-99, each of which is herein incorporated by reference in it's entirety). In one embodiment, methionine residues of TCR/TRIOPEP peptides are modified.

In certain embodiments, TABLE 2 demonstrates the following structures of representative TCR-related trifunctional peptides: TCR/TRIOPEP LA32 (LGKATLYAVLPLGEEMRDRARAHVDALRTHLA) (SEQ ID NO. 17) and TCR/TRIOPEP LE32 (LGKATLYAVLPYLDDFQKKWQEEMELYRQKVE) (SEQ ID NO. 18). While not being bound to any particular theory, it is believed that in one embodiment, these peptides colocalize with TCR in the cell membrane and selectively disrupt intramembrane interactions of TCRb chain with the CD3eg heterodimer, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in it's entirety).

In certain embodiments, TABLE 2 demonstrates the following structures of representative TCR-related trifunctional peptides: TCR/TRIOPEP YA32 (YLLDGILFIYPLGEEMRDRARAHVDALRTHLA) (SEQ ID NO. 19) and TCR/TRIOPEP YE32 (YLLDGILFIYPYLDDFQKKWQEEMELYRQKVE) (SEQ ID NO. 20). While not being bound to any particular theory, it is believed that in one embodiment, these peptides colocalize with TCR in the cell membrane and selectively disrupt intramembrane interactions of CD3zz homodimer with TCRa chain, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in it's entirety).

In certain embodiments, TABLE 2 demonstrates the following structures of representative TCR-related trifunctional peptides: TCR/TRIOPEP IA32 (IIVTDVIATLPLGEEMRDRARAHVDALRTHLA) (SEQ ID NO. 21) and TCR/TRIOPEP IE32 (IIVTDVIATLPYLDDFQKKWQEEMELYRQKVE) (SEQ ID NO. 22). While not being bound to any particular theory, it is believed that in one embodiment, these peptides colocalize with TCR in the cell membrane and selectively disrupt intramembrane interactions of CD3ed heterodimer with TCRa chain, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in it's entirety).

In certain embodiments, TABLE 2 demonstrates the following structures of representative TCR-related trifunctional peptides: TCR/TRIOPEP FA32 (FLFAEIVSIFPLGEEMRDRARAHVDALRTHLA) (SEQ ID NO. 23) and TCR/TRIOPEP FE32 (FLFAEIVSIFPYLDDFQKKWQEEMELYRQKVE) (SEQ ID NO. 24). While not being bound to any particular theory, it is believed that in one embodiment, these peptides colocalize with TCR in the cell membrane and selectively disrupt intramembrane interactions of CD3eg heterodimer with TCRb chain, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, each of which is herein incorporated by reference in it's entirety).

In certain embodiments, TABLE 2 demonstrates the following structures of representative TCR-related trifunctional peptides: TCR/TRIOPEP IA32e (IVIVDICITGPLGEEMRDRARAHVDALRTHLA) (SEQ ID NO. 25) and TCR/TRIOPEP IE32e (IVIVDICITGPYLDDFQKKWQEEMELYRQKVE) (SEQ ID NO. 26). While not being bound to any particular theory, it is believed that in one embodiment, these peptides colocalize with TCR in the cell membrane and selectively disrupt intramembrane interactions of CD3eg and CD3ed heterodimers with TCRb and TCRa chains, respectively, resulting to specific ligand-independent inhibition of TCR upon antigen stimulation (see e.g. Sigalov. Self Nonself 2010, 1:4-39; Sigalov. Self Nonself 2010, 1:192-224; and Sigalov. Adv Protein Chem Struct Biol 2018, 111:61-99, all of which are herein incorporated by reference in their entirety).

In one embodiment, methionine residues of TCR/TRIOPEP peptides are modified as described herein.

In certain embodiments, the capability of the TCR-related peptides and compounds of the present invention including but not limiting to those described above, to inhibit TCR can be used to treat and/or prevent TCR-related diseases and conditions including but not limiting to, allergic diathesis e.g. delayed type hypersensitivity, contact dermatitis; autoimmune disease e.g. systemic lupus erythematosus, rheumatoid arthritis, multiple sclerosis, diabetes, Guillain-Barre syndrome, Hashimotos disease, pernicious anaemia; gastroenterological conditions e.g. inflammatory bowel disease, Crohn's disease, primary biliary cirrhosis, chronic active hepatitis; skin problems e.g. atopic dermatitis, psoriasis, pemphigus vulgaris; infective disease; respiratory conditions e.g. allergic alveolitis; cardiovascular problems e.g. autoimmune pericarditis; organ transplantation; inflammatory conditions e.g. myositis, ankylosing spondylitis; and any other disorder where T cells are involved/recruited

In certain embodiments, the capability of the TCR-related peptides and compounds of the present invention including but not limiting to those described above, to colocalize with TCR can be used to visualize (image) this receptor and evaluate its expression in the areas of interest. In one embodiment, for this purpose, an imaging probe (e.g. [⁶⁴Cu], see TABLE 2) can be conjugated to the TCR/TRIOPEP sequences In one embodiment, imaging (visualization) of TCR levels using PET and/or other imaging techniques can be used to diagnose TCR-related diseases and conditions as well as to monitor novel therapies for these diseases and conditions.

C. NKG2D-Related Trifunctional Peptides

NKG2D is an activating receptor expressed by natural killer (NK) and T cells. The NKG2D is a complex of an NKG2D chain, which is responsible for ligand recognition, and DAP10 homodimer, which is responsible for transmembrane signal transduction (see e.g. Sigalov. Trends Pharmacol Sci 2006, 27:518-524; Sigalov. Trends Immunol 2004, 25:583-589; Sigalov. Self Nonself 2010, 1:4-39; and Sigalov. Self Nonself 2010, 1:192-224, all of which are herein incorporated by reference in their entirety). NKG2D ligands show a restricted expression in normal tissues, but they are frequently overexpressed in cancer and infected cells. The binding of NKG2D to its ligands activates NK and T cells and promotes cytotoxic lysis of the cells expressing these molecules. The mechanisms involved in the expression of the ligands of NKG2D play a role in the recognition of stressed cells by the immune system and represent a promising therapeutic target for improving the immune response against cancer or autoimmune disease (see e.g. Gonzalez, et al. Trends Immunol 2008, 29:397-403; Ito, et al. Am J Physiol Gastrointest Liver Physiol 2008, 294:G199-207; Vilarinho, et al. Proc Natl Acad Sci USA 2007, 104:18187-18192; Van Belle, et al. J Autoimmun 2013, 40:66-73; Lopez-Soto, et al. Int J Cancer 2015, 136:1741-1750; and Urso, et al. U.S. Pat. No. 9,127,064, each of which is herein incorporated by reference in it's entirety).

The preferred NKG2D-related peptides and compositions of this class comprise the domain A comprising the NKG2D modulatory peptide sequences designed using a well-known in the art novel model of cell receptor signaling, the Signaling Chain HOmoOLigomerization model, capable of modulating NKG2D (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9, all of which are herein incorporated by reference in their entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety).

As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative NKG2D-related trifunctional peptides: NKG2D/TRIOPEP IA36 (IAVAMGIRFIIMVAPLGEEMRDRARAHVDALRTHLA) (SEQ ID NO. 27) and NKG2D/TRIOPEP IE36 (IAVAMGIRFIIMVAPYLDDFQKKWQEEMELYRQKVE) (SEQ ID NO. 28). While not being bound to any particular theory, it is believed that in one embodiment, these peptides colocalize with NKG2D in the cell membrane and selectively disrupt intramembrane interactions of NKG2D chain with the DNAX-activation protein 10 (DAP-10) signaling homodimer, resulting to specific ligand-independent inhibition of NKG2D upon ligand stimulation (see e.g. Sigalov. Self Nonself 2010, 1:4-39.; Sigalov. Self Nonself 2010, 1:192-224; and Sigalov. Adv Protein Chem Struct Biol 2018, 111:61-99, all of which are herein incorporated by reference in their entirety). In one embodiment, methionine residues of NKG2D/TRIOPEP peptides are modified.

In certain embodiments, the capability of the NKG2D-related peptides and compounds of the present invention including but not limiting to those described above, to inhibit NKG2D can be used to treat and/or prevent NKG2D-related diseases and conditions including but not limiting to, celiac disease, type I diabetes, hepatitis, and rheumatoid arthritis, and any other disorder where NKG2D cells are involved/recruited. In one embodiment, the present invention provides methods and compositions for preventing NK cell-mediated graft rejection.

In certain embodiments, the capability of the NKG2D-related peptides and compounds of the present invention including but not limiting to those described above, to colocalize with NKG2D can be used to visualize (image) this receptor and evaluate its expression in the areas of interest. In one embodiment, for this purpose, an imaging probe (e.g. [⁶⁴Cu], see TABLE 2) can be conjugated to the NKG2D/TRIOPEP sequences In one embodiment, imaging (visualization) of NKG2D levels using PET and/or other imaging techniques can be used to diagnose NKG2D-related diseases and conditions as well as to monitor novel therapies for these diseases and conditions.

D. GPVI-Related Trifunctional Peptides

In recent years, the central activating platelet collagen receptor, glycoprotein (GP) VI, has emerged as a promising antithrombotic target because its blockade or antibody-mediated depletion in circulating platelets was shown to effectively inhibit experimental thrombosis and thromboinflammatory disease states, such as stroke, without affecting hemostatic plug formation. GPVI is a complex of a GPVI chain, which is responsible for ligand recognition, and FcRg homodimer, which is responsible for transmembrane signal transduction (see e.g. Sigalov. Trends Pharmacol Sci 2006, 27:518-524; Sigalov. Trends Immunol 2004, 25:583-589; Sigalov. Self Nonself 2010, 1:4-39; Sigalov. Self Nonself 2010, 1:192-224; Sigalov. J Thromb Haemost 2007, 5:2310-2312; Sigalov. Expert Opin Ther Targets 2008, 12:677-692; Dutting, et al. Trends Pharmacol Sci 2012, 33:583-590; Ungerer, et al. PLoS One 2013, 8:e71193; Sigalov, U.S. Pat. No. 8,278,271; Sigalov, U.S. Pat. No. 8,614,188, all of which are herein incorporated by reference in their entirety). The binding of GPVI to collagen or other antagonists ligands induces platelet adhesion, activation and aggregation. Platelet activation is a step in the pathogenesis of ischemic cardio- and cerebrovascular diseases, which represent the leading causes of death and severe disability worldwide. Although existing antiplatelet drugs have proved beneficial in the clinic, their use is limited by their inherent effect on primary hemostasis, making the identification of novel pharmacological targets for platelet inhibition a goal of cardiovascular research.

The preferred GPVI-related peptides and compositions of this class comprise the domain A comprising the GPVI modulatory peptide sequences designed using a well-known in the art novel model of cell receptor signaling, the Signaling Chain HOmoOLigomerization model, capable of modulating GPVI (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9; Sigalov. J Thromb Haemost 2007, 5:2310-2312; Sigalov. Expert Opin Ther Targets 2008, 12:677-692, each of which is herein incorporated by reference in it's entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative GPVI-related trifunctional peptides: GPVI/TRIOPEP GA32 (GNLVRICLGAPLGEEMRDRARAHVDALRTHLA) (SEQ ID NO. 29) and GPVI/TRIOPEP GE32 (GNLVRICLGAPYLDDFQKKWQEEMELYRQKVE) (SEQ ID NO. 30). While not being bound to any particular theory, it is believed that in one embodiment, these peptides colocalize with GPVI in the cell membrane and selectively disrupt intramembrane interactions of GPVI chain with the FcRg signaling homodimer, resulting to specific ligand-independent inhibition of GPVI upon ligand stimulation (see e.g. Sigalov. Self Nonself 2010, 1:4-39.; Sigalov. Self Nonself 2010, 1:192-224; Sigalov. Adv Protein Chem Struct Biol 2018, 111:61-99; Sigalov. J Thromb Haemost 2007, 5:2310-2312; Sigalov. Expert Opin Ther Targets 2008, 12:677-692, each of which is herein incorporated by reference in it's entirety). In one embodiment, methionine residues of GPVI/TRIOPEP peptides are modified.

In certain embodiments, the capability of the GPVI-related peptides and compounds of the present invention including but not limiting to those described above, to inhibit GPVI can be used to treat and/or prevent GPVI-related diseases and conditions including but not limiting to, ischemic and thromboinflammatory diseases, and any other disorder where platelets are involved/recruited.

In certain embodiments, the capability of the GPVI-related peptides and compounds of the present invention including but not limiting to those described above, to colocalize with GPVI can be used to visualize (image) this receptor and evaluate its expression in the areas of interest. In one embodiment, for this purpose, an imaging probe (e.g. [⁶⁴Cu], see TABLE 2) can be conjugated to the GPVI/TRIOPEP sequences In one embodiment, imaging (visualization) of GPVI levels using PET and/or other imaging techniques can be used to diagnose GPVI-related diseases and conditions as well as to monitor novel therapies for these diseases and conditions.

E. DAP-10- and DAP-12-Related Trifunctional Peptides

The DAP10 and DAP12 signaling subunits are highly conserved in evolution and associate with a large family of receptors in hematopoietic cells, including dendritic cells, plasmacytoid dendritic cells, neutrophils, basophils, eosinophils, mast cells, monocytes, macrophages, natural killer cells, and some B and T cells. Some receptors are able to associate with either DAP10 or DAP12, which contribute unique intracellular signaling functions. DAP-10- and DAP-12-associated receptors have been shown to recognize both host-encoded ligands and ligands encoded by microbial pathogens, indicating that they play a role in innate immune responses. See e.g. Lanier. Immunol Rev 2009, 227:150-160; Sigalov. Trends Pharmacol Sci 2006, 27:518-524; Sigalov. Trends Immunol 2004, 25:583-589; Sigalov. Self Nonself 2010, 1:4-39; Sigalov. Self Nonself 2010, 1:192-224, all of which are herein incorporated by reference in their entirety.

The preferred DAP-10 and DAP-12-related peptides and compositions of this class comprise the domain A comprising the DAP-10 or DAP-12 modulatory peptide sequences, respectively, designed using a well-known in the art novel model of cell receptor signaling, the Signaling Chain HOmoOLigomerization model, capable of modulating DAP-10- and DAP-12-associated receptors (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9, each of which is herein incorporated by reference in it's entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety).

As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative DAP-10-related trifunctional peptides: DAP-10/TRIOPEP LA32 (LVAADAVASLPLGEEMRDRARAHVDALRTHLA) (SEQ ID NO. 33) and DAP-10/TRIOPEP LE32 (LVAADAVASLPYLDDFQKKWQEEMELYRQKVE) (SEQ ID NO. 34). While not being bound to any particular theory, it is believed that in one embodiment, these peptides colocalize with DAP-10-associated cell receptors in the cell membrane and selectively disrupt intramembrane interactions of the receptor with the DAP-10 signaling homodimer, resulting to specific ligand-independent inhibition of the receptor upon ligand stimulation (see e.g. Sigalov. Self Nonself 2010, 1:4-39.; Sigalov. Self Nonself 2010, 1:192-224; Sigalov. Adv Protein Chem Struct Biol 2018, 111:61-99, each of which is herein incorporated by reference in it's entirety). In one embodiment, methionine residues of DAP-10/TRIOPEP peptides are modified.

In certain embodiments, TABLE 2 demonstrates the following structures of representative DAP-12-related trifunctional peptides: DAP-12/TRIOPEP VA32 (VMGDLVLTVLPLGEEMRDRARAHVDALRTHLA) (SEQ ID NO. 31) and DAP-12/TRIOPEP VE32 (VMGDLVLTVLPYLDDFQKKWQEEMELYRQKVE) (SEQ ID NO. 32). While not being bound to any particular theory, it is believed that in one embodiment, these peptides colocalize with DAP-12-associated cell receptors in the cell membrane and selectively disrupt intramembrane interactions of the receptor with the DAP-12 signaling homodimer, resulting to specific ligand-independent inhibition of the receptor upon ligand stimulation (see e.g. Sigalov. Self Nonself 2010, 1:4-39.; Sigalov. Self Nonself 2010, 1:192-224; Sigalov. Adv Protein Chem Struct Biol 2018, 111:61-99, each of which is herein incorporated by reference in it's entirety). In one embodiment, methionine residues of DAP-12/TRIOPEP peptides are modified.

In certain embodiments, the capability of the DAP-10- and DAP-12-related peptides and compounds of the present invention including but not limiting to those described above, to inhibit the DAP-10- and DAP-12-associated receptors, respectively, can be used to treat and/or prevent any diseases and conditions where these receptors are involved.

In certain embodiments, the capability of the DAP-10- and DAP-12-related peptides and compounds of the present invention including but not limiting to those described above, to colocalize with the DAP-10- and DAP-12-associated receptors, respectively, can be used to visualize (image) these receptors and evaluate their expression in the areas of interest. In one embodiment, for this purpose, an imaging probe (e.g. [⁶⁴Cu], see TABLE 2) can be conjugated to the DAP-10/TRIOPEP and DAP-12/TRIOPEP sequences. In one embodiment, imaging (visualization) of levels of the DAP-10- and DAP-12-associated receptors using PET and/or other imaging techniques can be used to diagnose any diseases and conditions where these receptors are involved as well as to monitor novel therapies for these diseases and conditions.

6. EGFR-Related Trifunctional Peptides

The epidermal growth factor (EGF) receptor (EGFR) family, or ErbB family, is the best studied example of oncogenic receptor tyrosine kinases (RTKs). HER2/ErbB2 is overexpressed on the surface of 25-30% of breast cancer cells, and it has been associated with a high risk of relapse and death. EGFR amplification and mutations have been associated with many carcinomas. In particular, the EGFR pathway appears to play a role in pancreatic carcinoma. See e.g. Overholser, et al. Cancer 2000, 89:74-82; Bennasroune, et al. Mol Biol Cell 2004, 15:3464-3474; Sigalov. Trends Pharmacol Sci 2006, 27:518-524; Sigalov. Trends Immunol 2004, 25:583-589; Sigalov. Self Nonself 2010, 1:4-39; Sigalov. Self Nonself 2010, 1:192-224, each of which is herein incorporated by reference in it's entirety. Short hydrophobic peptides corresponding to the transmembrane domains of EGFR, ErB2 and insulin receptors inhibit specifically the autophosphorylation and signaling pathway of their cognate receptor (see Bennasroune, et al. Mol Biol Cell 2004, 15:3464-3474, all of which are herein incorporated by reference in their entirety).

The preferred EGFR-related peptides and compositions of this class comprise the domain A comprising the EGFR modulatory peptide sequences, designed using a well-known in the art novel model of cell receptor signaling, the Signaling Chain HOmoOLigomerization model, capable of modulating EGFR (see e.g., Sigalov. Self Nonself 2010, 1:4-39; Sigalov. Self Nonself 2010, 1:192-224; Sigalov. Trends Pharmacol Sci 2006, 27:518-524; Sigalov. Trends Immunol 2004, 25:583-589; Sigalov. Adv Protein Chem Struct Biol 2018, 111:61-9, all of which are herein incorporated by reference in their entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety).

As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In one embodiment, TREM-1 inhibitory SCHOOL peptide GF9 described herein is incorporated into SLP that contain apo A-I peptide fragments comprising 22 amino acid residue-long peptide sequences of the apo A-I helix 4 and/or helix 6. In one embodiment, the inclusion of an amphipathic apo A-I sequences in the peptides and compositions of the invention further aids the ability to provide targeted delivery to the cells of interest. It further aids the ability to interact with lipids and/or lipoproteins in a bloodstream in vivo and form lipopeptide particles (LP) that mimic native lipoproteins. It further aids the ability to cross the blood-brain barrier (BBB), blood-retinal barrier (BRB) and blood-tumor barrier (BTB).

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative EGFR-related trifunctional peptides: EGFR/TRIOPEP SA47 (SIATGMVGALLLLLVVALGIGLFMRPLGEEMRDRARAHVDALRTHLA) (SEQ ID NO. 35) and EGFR/TRIOPEP SE47 (SIATGMVGALLLLLVVALGIGLFMRPYLDDFQKKWQEEMELYRQKVE) (SEQ ID NO. 36). While not being bound to any particular theory, it is believed that in one embodiment, these peptides colocalize with EGFR in the cell membrane and selectively disrupts intramembrane interactions between the receptors, resulting to specific ligand-independent inhibition of the receptor (see e.g. Sigalov. Self Nonself 2010, 1:4-39.; Sigalov. Self Nonself 2010, 1:192-224; Sigalov. Adv Protein Chem Struct Biol 2018, 111:61-99, all of which are herein incorporated by reference in their entirety). In one embodiment, methionine residues of EGFR/TRIOPEP peptides are modified.

In certain embodiments, the capability of the EGFR and/or ErB-related peptides and compounds of the present invention including but not limiting to those described above, to inhibit the receptors of the EGFR and/or ErB receptor families, respectively, can be used to treat and/or prevent any diseases and conditions where these receptors are involved.

In certain embodiments, the capability of the EGFR- and/or ErB-related peptides and compounds of the present invention including but not limiting to those described above, to colocalize with the receptors of the EGFR and/or ErB receptor families can be used to visualize (image) these receptors and evaluate their expression in the areas of interest. In one embodiment, for this purpose, an imaging probe (e.g. [⁶⁴Cu]) can be conjugated to the EGFR/TRIOPEP sequence. In one embodiment, imaging (visualization) of levels of the receptors of the EGFR and/or ErB receptor families using PET and/or other imaging techniques can be used to diagnose any diseases and conditions where these receptors are involved as well as to monitor novel therapies for these diseases and conditions.

F. Additional Trifunctional Peptides

Additional therapeutic peptide sequences (see e.g., Vlieghe, et al. Drug Discov Today 2010, 15:40-56; Tsung, et al. Shock 2007, 27:364-369; Chang, et al. PLoS One 2009, 4:e4171; Tjin Tham Sjin, et al. Cancer Res 2005, 65:3656-3663; Ladetzki-Baehs, et al. Endocrinology 2007, 148:332-336; Khan, et al. Hum Immunol 2002, 63:1-7; Banga. Therapeutic peptides and proteins: formulation, processing, and delivery systems. 2nd ed. Boca Raton, Fla.: Taylor & Francis Group; 2006; Stevenson. Curr Pharm Biotechnol 2009, 10:122-137; Wu and Chi, U.S. Pat. No. 9,387,257; Wu, et al., U.S. Pat. No. 8,415,453; Faure, et al., U.S. Pat. No. 8,013,116; Faure, et al., U.S. Pat. No. 9,273,111; Eggink and Hoober, U.S. Pat. No. 7,811,995; Eggink and Hoober, U.S. Pat. No. 8,496,942; Morgan and Pandha. US 2012/0177672 A1; Broersma, et al., U.S. Pat. No. 5,681,925, each of which is herein incorporated by reference in it's entirety) and/or other therapeutic agents can comprise the domain A of the peptides and compositions of the present invention.

In one embodiment, this domain comprises the Toll Like Receptor (TLR) modulatory sequence (see e.g. Tsung, et al. Shock 2007, 27:364-369, herein incorporated by reference in it's entirety). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, each of which is herein incorporated by reference in it's entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative TLR-related trifunctional peptides: TLR/TRIOPEP DA32 (DIVKLTVYDCIRRRRRRRRRPLGEEMRDRARAHVDALRTHLA) (SEQ ID NO. 37) and TLR/TRIOPEP DE32 (DIVKLTVYDCIRRRRRRRRRPYLDDFQKKWQEEMELYRQKVE) (SEQ ID NO. 38). In one embodiment, methionine residues of TLR/TRIOPEP peptides are modified.

In certain embodiments, the capability of the TLR-related peptides and compounds of the present invention including but not limiting to those described above, to inhibit TLR can be used to treat and/or prevent TLR-related diseases and conditions including but not limiting to, sepsis and other infectious diseases, and any other disorder where TLR receptors are involved.

In certain embodiments, the capability of the TLR-related peptides and compounds of the present invention including but not limiting to those described above, to colocalize with TLR can be used to visualize (image) this receptor and evaluate its expression in the areas of interest. In one embodiment, for this purpose, an imaging probe (e.g. [⁶⁴Cu]) can be conjugated to the TLR/TRIOPEP sequences In one embodiment, imaging (visualization) of TLR levels using PET and/or other imaging techniques can be used to diagnose TLR-related diseases and conditions as well as to monitor novel therapies for these diseases and conditions.

In one embodiment, the domain A of the peptides and compositions of the invention comprises the Atrial Natriuretic Peptide (ANP) receptor (ANPR)-modulatory sequence (see e.g. Ladetzki-Baehs, et al. Endocrinology 2007, 148:332-336). The preferred peptides and compositions of this class further comprise the domain B comprising at least one modified or unmodified amphipathic apo A-I and/or A-II peptide fragment to form upon interaction with lipid and/or lipid mixtures, SLP structures that can be spherical or discoidal (described herein and in e.g., Sigalov. Contrast Media Mol Imaging 2014, 9:372-382; Sigalov. Int Immunopharmacol 2014, 21:208-219; Sigalov. US 20110256224; Sigalov. US 20130045161; Shen, et al. PLoS One 2015, 10:e0143453; Shen and Sigalov. Sci Rep 2016, 6:28672; Shen and Sigalov. J Cell Mol Med 2017, 21:2524-2534; Shen and Sigalov. Mol Pharm 2017, 14:4572-4582; Rojas, et al. Biochim Biophys Acta 2018, 1864:2761-2768, all of which are herein incorporated by reference in their entirety). As described above, the inclusion of an amphipathic apo A-I sequences aids the assistance in the self-assembly of SLP and the structural stability of the particle formed, particularly when the particle has a discoidal shape. It further aids the ability to provide targeted delivery to the cells of interest. It further aids the ability to interact with lipids and/or lipoproteins in a bloodstream in vivo and form LP that mimic native lipoproteins. It further aids the ability to cross the BBB, BRB and BTB.

In certain embodiments, exemplary trifunctional peptides comprise the domain B comprises with the amino acid sequence selected from the amino acid sequences of the major HDL protein constituent, apo A-I. In certain embodiments, this sequence comprises 22 amino acid residue-long peptide sequence of the apo A-I helix 4. In one embodiment, this sequence contains a modified amino acid residue. In one embodiment, this modified amino acid residue is methionine sulfoxide. In one embodiment, the domain B of the peptides and compositions of the invention comprises 22 amino acid residue-long peptide sequence of the apo A-I helix 6. In one embodiment, this sequence contains a modified amino acid residue. In one embodiment, this modified amino acid residue is methionine sulfoxide.

In certain embodiments, TABLE 2 demonstrates the following structures of representative ANPR-related trifunctional peptides: ANPR/TRIOPEP SA50 (SLRRSSCFGGRMDRIGAQSGLGCNSFRYPLGEEMRDRARAHVDALRTHLA) (SEQ ID NO. 39) and ANPR/TRIOPEP SE50 (SLRRSSCFGGRMDRIGAQSGLGCNSFRYPYLDDFQKKWQEEMELYRQKVE) (SEQ ID NO. 40). In one embodiment, methionine residues of ANPR/TRIOPEP peptides are modified.

In certain embodiments, the capability of the ANPR-related peptides and compounds of the present invention including but not limiting to those described above, to inhibit ANPRs can be used to treat and/or prevent ANPR-related diseases and conditions including but not limiting to, cardiovascular and inflammatory diseases, and any other disorder where ANP receptors are involved.

In certain embodiments, the capability of the ANPR-related peptides and compounds of the present invention including but not limiting to those described above, to colocalize with ANPR can be used to visualize (image) this receptor and evaluate its expression in the areas of interest. In one embodiment, for this purpose, an imaging probe (e.g. [⁶⁴Cu]) can be conjugated to the ANPR/TRIOPEP sequences In one embodiment, imaging (visualization) of ANPR levels using PET and/or other imaging techniques can be used to diagnose ANPR-related diseases and conditions as well as to monitor novel therapies for these diseases and conditions.

In certain embodiments, other therapeutic agents including but not limiting to, to those described in Page and Takimoto. Principles of chemotherapy. In: Pazdur R, Wagman L D, Camphausen K A, editors. Cancer Management: A Multidisciplinary Approach. 11th ed. Manhasset, N.Y.: Cmp United Business Media; 2009. p. 21-37; Sipsas, et al., Therapy of Mucormycosis, J Fungi (Basel) 2018, 4; Lin, et al. Chem Commun (Camb) 2013, 49:4968-4970; and in Turner, et al. Peptide Conjugates of Oligonucleotide Analogs and siRNA for Gene Expression Modulation. In: Langel U, ed, editor. Handbook of Cell-Penetrating Peptides. 2nd edition ed. Boca Raton: CRC Press; 2007. p. 313-328 and disclosed in Schiffman and Altman, U.S. Pat. No. 4,427,660; Castaigne, et al., U.S. Pat. No. 9,161,988; Castaigne, et al., U.S. Pat. No. 8,921,314; and in Castaigne, et al., U.S. Pat. No. 9,173,891, all of which are herein incorporated by reference in their entirety, (see also TABLE 2) can comprise the domain A of the peptides and compositions of the present invention.

### V. Diseases Contemplated for Treatment Using Peptides and Compositions Described Herein.

A. Overview.

The present invention encompasses the recognition that it is possible to produce compositions that possess the advantages typically associated with a fully synthetic material and yet also possess certain desirable features of materials derived from natural sources.

In some embodiments, peptides and compounds of the present invention, e.g. trifunctional peptides, with rHDLs (including discoidal and/or spherical HDLs) or without rHDLs (such as in therapeutic compositions as free trifunctional peptides), are contemplated for use in preventative treatments for diseases associated with activated macrophages and/or T-cells, in particular for preventing one or more symptoms associated with the disease. In some embodiments, peptides and compounds of the present invention are contemplated for use preventative treatments for diseases associated with activated macrophages and/or T-cells, in particular for reducing one or more symptoms associated with the disease. In some embodiments, peptides and compounds of the present invention are contemplated for use diagnostic applications for detecting/identifying; determining disease progression; determining results of disease treatment, for diseases associated with activated macrophages and/or T-cells. Such diseases associated with activated macrophages and/or T-cells include but are not limited to including but not limited to lung cancer, such as non small-cell lung cancer (NSCLC); pancreatic cancer (PC); glioblastoma multiforme (GBM, or brain cancer), with or without radiation therapy; breast cancer with or without radiation therapy; sepsis; retinopathy; rheumatoid arthritis (RA); sepsis; and alcoholic liver disease (ALD). Furthermore, diseases associated with activated macrophages and/or T-cells include but are not limited to 1) Alcohol-induced neuroinflammation and brain damage; 2) Radiation-induced multiple organ dysfunction syndrome; 3) Scleroderma; 4) Atopic dermatitis; 5) Atherosclerosis; 6) Alzheimer's, Parkinson's and/or Huntington's diseases. In one embodiment, the present invention relates to the targeted treatment, prevention and/or detection of cancer including but not limited to lung, pancreatic, breast, stomach, prostate, colon, brain and skin cancers, cancer cachexia, atherosclerosis, allergic diseases, acute radiation syndrome, inflammatory bowel disease, empyema, acute mesenteric ischemia, hemorrhagic shock, multiple sclerosis, liver diseases, autoimmune diseases, including but not limited to, atopic dermatitis, lupus, scleroderma, rheumatoid arthritis, psoriatic arthritis and other rheumatic diseases, sepsis and other inflammatory diseases or other condition involving myeloid cell activation and, more particularly, TREM receptor-mediated cell activation, including but not limited to diabetic retinopathy and retinopathy of prematurity, Alzheimer's, Parkinson's and Huntington's diseases.

Thus, in some embodiments, trifunctonal peptides as described herein are contemplated for administration to a subject for reducing a disease symptom. In some embodiments, trifunctonal peptides as described herein are contemplated for administration to a subject for delaying onset of a disease symptom. In some embodiments, trifunctonal peptides as described herein are contemplated for administration to a subject for preventing a disease symptom. In some embodiments, trifunctonal peptides as described herein are contemplated for administration to a subject receiving therapy for a disease. In some embodiments, trifunctonal peptides as described herein are contemplated for administration to a subject receiving anti-cancer therapy. In some embodiments, trifunctonal peptides as described herein, are contemplated for administration to a subject as anti-cancer therapy. In some embodiments, trifunctonal peptides as described herein, further comprising a drug compound are contemplated for administration to a subject as anti-cancer therapy. In some embodiments, trifunctonal peptides as described herein, further comprising a Paclitaxel compound are contemplated for administration to a subject as anti-cancer therapy.

As disease progression of a liver in a subject proceeds from epatosteatosis, steatohepatitis, and fibrosis to cirrhosis, it is contemplated that a trifunctonal peptide as described herein, is administered to said subject at any point along the disease progression for reducing disease progression, in part as described herein. Thus, in some embodiments, trifunctonal peptides as described herein are contemplated for administration to a subject for reducing a liver disease symptom, including but not limited to reducing one or more of ALT, procollegen I-alpha and alpha-SMA.

In some embodiments, trifunctonal peptides as described herein are contemplated for administration to a subject for reducing a liver disease symptom, in combination with one or more of steroid drugs, ursodiol, etc., in order to delay or prevent further progression of liver degeneration to cirrhosis. In some embodiments, trifunctonal peptides as described herein are contemplated for administration to a subject for reducing a liver disease symptom in combination with one or more of steroid drugs, ursodiol, etc., in order to improve function of a diseased liver.

In some embodiments, trifunctonal peptides as described herein are contemplated for administration to a subject for treating severe hemorrhagic shock. In some embodiments, trifunctonal peptides as described herein are contemplated for administration to a subject for treating colitis and colitis-associated tumorigenesis.

In some embodiments, trifunctonal peptides as described herein are contemplated for administration to a subject for decreasing neovascularization.

In some embodiments, trifunctonal peptides as described herein are selected from the group consisting of G-KV21, G-HV21, G-TE21, M-VE32 and M-TK32, and mixtures thereof. In some embodiments, a trifunctonal peptide as described herein is GE31. In some embodiments, a trifunctonal peptide as described herein is GA31. In some embodiments, a trifunctonal peptide as described herein is a mixture of GE31 and GA31.

FIG. 4B illustrates a hypothesized molecular mechanism of action of one embodiment of a trifunctional peptide (TRIOPEP) of the present invention comprising amino acid domains A and B where domain A is a 9 amino acids-long TREM-1 inhibitory therapeutic peptide sequence and functions to treat and/or prevent a TREM-1-related disease or condition (shown for cancer), whereas domain B is a 22 amino acids-long apolipoprotein A-I helix 4 or 6 peptide sequence with a sulfoxidized methionine residue and functions to assist in the self-assembly of synthetic lipopeptide particles (SLP) and to target the particles to TREM-1-expressing macrophages as applied to the treatment and/or prevention of cancer. While not being bound to any particular theory, it is believed that chemical and/or enzymatic modification of protein sequence in domain B leads to the recognition of SLP of the present invention by the macrophage scavenger receptors and results in an irreversible binding to and consequent uptake by macrophages of such particles. It is further believed that accumulation of these particles in tumor-associated macrophages is accompanied by accumulation of TRIOPEP in these cells. In contrast, native HDL particles that contain only unmodified apolipoprotein molecules are not recognized by tumor-associated macrophages and return to the circulation.

FIG. 4C shows a symbol key used in FIGS. 4A-B.

B. Cancer.

Approximately 8.8 million people are dying each year of cancer, amounting to one out of six deaths globally, and cancer incidence is estimated to double by 2035 (Prager et al. 2018). Combination-therapy treatments for cancer have become more common, in part due to the perceived advantage of attacking the disease via multiple avenues. Although many effective combination-therapy treatments have been identified over the past few decades; in view of the continuing high number of deaths each year resulting from cancer, a continuing need exists to identify effective therapeutic regimens for use in anticancer treatment.

The present invention encompasses the recognition that it is possible to prevent and treat different types of cancer including but not limited to, pancreatic cancer, multiple myeloma, leukemia, prostate cancer, breast cancer, liver cancer, bladder cancer, colorectal cancer, lung cancer, CNS cancer, melanoma, ovarian cancer, renal cancer, or osteosarcoma and other cancers, and cancer cachexia by blocking the TREM-1 signaling pathway using the peptide variants and compositions that possess the advantages typically associated with a fully synthetic material and yet possess certain desirable features of materials derived from natural sources. The invention further encompasses the recognition that it is possible to use imaging techniques and the peptide variants and compositions of the invention conjugated to an imaging probe for detecting the labeled probe in an individual with cancer, wherein the location of the labeled probe corresponds to at least one symptom of the myeloid cell-related condition. The invention further encompasses that it is possible to predict the efficacy of the peptides and compositions of the invention by determining the number of myeloid cells in the biological sample from the individual with cancer and determining the expression levels of TREM-1 in the cells contained within the biological sample.

Cancer continues to have a huge Social and economic impact. In 2011, 571,950 Americans died of cancer (−25% of all deaths), with US cancer-associated costs of S263.8 billion: S102.8 billion for direct medical costs (total health expenditures); $20.9 billion for indirect morbidity costs (lost productivity); and S140.1 billion for indirect mortality costs (lost productivity from premature death).

Inflammatory responses play decisive roles at different stages of tumor development, including initiation, promotion, malignant conversion, invasion, and metastasis (Grivennikov et al. 2010). Inflammation also affects immune surveillance and responses to therapy (Grivennikov et al. 2010). Many solid tumors are characterized by a marked infiltration of macrophages, inflammatory cells, into the stromal compartment (Shih et al. 2006, Solinas et al. 2009), a process which is mediated by cancer-associated fibroblasts (CAFs) and plays a key role in disease progression and its response to therapy (see FIG. 49). These tumor-associated macrophages (TAMs) secrete a variety of growth factors, cytokines, chemokines, and enzymes that regulate tumor growth, angiogenesis, invasion, and metastasis (Shih et al. 2006). See FIG. 49. High macrophage infiltration correlates with the promotion of tumor growth and metastasis development (Solinas et al. 2009, Grivennikov et al. 2010). In patients with PC, macrophage infiltration begins during the preinvasive stage of the disease and increases progressively (Clark et al. 2007). The number of TAMs is significantly higher in patients with metastases (Gardian et al. 2012). TREM-1 is upregulated in cancer and its overexpression correlates with survival of cancer patients. In NSCLC, TREM-1 expression in TAMs is associated with cancer recurrence and poor survival of patients with NSCLC: patients with low TREM-1 expression have a 4-year survival rate of over 60%, compared with less than 20% in patients with high TREM-1 expression (Ho et al. 2008). Activation of the TREM-1/DAP-12 signaling pathway results in release of multiple cytokines, chemokines and growth factors most of which are increased in cancer patients and their upregulation correlates with poor prognosis (See FIG. 1).

The present invention encompasses the recognition that it is possible to prevent and treat different types of cancer in which myeloid cells are involved or recruited and cancer cachexia by combining cancer therapies with a therapeutically effective amount of at least one compound and/or composition (“modulator”) which affects myeloid cells by action on the TREM-1/DAP-12 signaling pathway.

The infiltrate of most solid tumors contains tumor-associated macrophages (TAMs) that are attracted by chemokines including CCL2 and represent attractive treatment targets in oncology (Shih et al. 2006, Mantovani et al. 2017). The increased TAM content in NSCLC (Yusen et al. 2018) is associated with poor prognosis in NSCLC (Welsh et al. 2005). TAM recruitment, activation, growth and differentiation are regulated by CSF-1 (Elgert et al. 1998, Laoui et al. 2014). Many tumor cells or tumor stromal cells have been found to produce CSF-1, which activates monocyte/macrophage cells through CSF-1 receptor (CSF-1R). The level of CSF-1 in tumors has been shown to correlate with the level of TAMs in the tumor. Higher levels of TAMs have been found to correlate with poorer patient prognoses in the majority of cancers. Increased pretreatment serum CSF-1 is a strong independent predictor of poor survival in NSCLC (Baghdadi et al. 2018). In addition, CSF-1 has been found to promote tumor growth and progression to metastasis in, for example, human breast cancer xenografts in mice (Paulus et al. 2006). Further, CSF-1R plays a role in osteolytic bone destruction in bone metastasis (Ohno et al. 2006). TAMs promote tumor growth, in part, by suppressing anti-tumor T cell effector function through the release of immunosuppressive cytokines and the expression of T cell inhibitory surface proteins. Blockade of CSF-1 or CSF-1R not only suppresses tumor angiogenesis and lymphangiogenesis (Kubota et al. 2009) but also improves response to T-cell checkpoint immunotherapies that target programmed cell death protein 1 (PD-1), programmed death-ligand 1 (PD-L1) and cytotoxic T lymphocyte antigen-4 (CTLA-4) (Zhu et al. 2014). Importantly, continuous CSF-1 inhibition affects pathological angiogenesis but not healthy vascular and lymphatic systems outside tumors (Kubota et al. 2009). In contrast to blockade of vascular endothelial growth factor (VEGF), interruption of CSF-1 inhibition does not promote rapid vascular regrowth (Kubota et al. 2009).

The present invention provides a method of treating these and other types of cancers by using modulators of the TREM-1/DAP-12 signaling pathway that are capable of binding TREM-1 and modulating TREM-1/DAP-12 receptor complex activity in combination-therapy treatments together with other cancer therapies. The invention further provides the methods for predicting response of a cancer patient to the treatment by using these modulators in combination-therapy regimen. These and other objects and advantages of the invention, as well as additional inventive features, will be apparent from the description of the invention provided herein.

The invention further encompasses the recognition that it is possible to predict response of the subject to the treatment by using the modulators of TREM-1/DAP-12 signaling pathway in combination-therapy regimen by: (a) obtaining a biological sample from the subject; (b) determining the expression of CSF-1, CSF-1R, IL-6, TREM-1 and/or number of CD68-positive TAMs or a combination thereof, wherein the higher is the expression of CSF-1, CSF-1R, IL-6, TREM-1 or the higher is number of CD68-positive TAMs or a combination thereof, the better the patient is predicted to respond to a therapy that involves the modulators.

The invention further encompasses the recognition that it is possible to use imaging techniques and the modulators conjugated to an imaging probe for detecting the labeled probe in an individual with cancer in which myeloid cells are involved or recruited, wherein the location and the measured intensity of the labeled probe can diagnose cancer and/or predict response of the subject to the treatment by using the modulators of TREM-1/DAP-12 signaling pathway, the higher the measured intensity of the labeled probe, the better the patient is predicted to respond to a therapy that involves the modulators. 1. Lung Cancer.

Lung cancer, including NSCLC, is the leading cause of cancer deaths worldwide (Wong et al. 2018) and has a poor prognosis. Despite advances made in chemotherapy, NSCLC is responsible for over 1.1 million deaths annually worldwide, and the 5-year survival rate for patients with NSCLC is reported to be only 15% or less than 18% (Zappa et al. 2016), showing an urgent need for new therapies.

FIG. 11A-B presents the exemplary data showing inhibition of tumor growth in the human non-small cell lung cancer (NSCLC) H292 (FIG. 11A) and A549 (FIG. 11B) xenograft mice treated with an equimolar mixture of the sulfoxidized methionine residue-containing TREM-1-related trifunctional peptides (TREM-1/TRIOPEP) GA 31 and GE 31 in free form. PTX, paclitaxel. ****, P≤0.0001 as compared with vehicle-treated animals.
FIG. 12A-B presents the exemplary data showing inhibition of tumor growth in the human non-small cell lung cancer H292 (FIG. 12A) and A549 (FIG. 12B) xenograft mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.
FIG. 12A-B presents the exemplary data showing inhibition of tumor growth in the human non-small cell lung cancer H292 (FIG. 12A) and A549 (FIG. 12B) xenograft mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.
FIG. 13 presents the exemplary data showing average tumor weights in the A549 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. PTX, paclitaxel. ****, p<0.0001 as compared with vehicle-treated animals.

2. Pancreatic Cancer.

Pancreatic cancer (PC, 85% of which are pancreatic ductal adenocarcinomas, PDAC) is the fourth leading cause of cancer-related mortality across the world with very poor clinical outcome. (Ilic et al. 2016). Current treatments of PC marginally prolong survival or relieve symptoms in patients with PC (Ilic and Ilic 2016). There has been no significant progress in the field of targeted therapy for PC (Walker et al. 2014) and despite tremendous efforts, the 5-year survival rate remains less than 5% (Ilic and Ilic 2016). This highlights the urgent need for novel approaches to prevent and treat PC and other types of cancer. However, it should be noted that the techniques and compositions listed and described herein are applicable to a broad range of other types of cancer and cancer cachexia. Other features and advantages of the invention will become apparent from the following detailed description. It should be understood, however, that the detailed description and the specific examples, while indicating preferred embodiments of the invention, are given by way of illustration, because various changes and modifications within the spirit and scope of the invention will become apparent to those skilled in the art from this detailed description.

Current treatments of PC marginally prolong survival or relieve symptoms in patients with PC (Schneider 2005). There has been no significant progress in the field of targeted therapy for PC (Walker and Ko 2014) and despite tremendous efforts, the 5-year survival rate remains less than 5% (2010).

3. Additional Neoplasms: Giant Cell Tumor and PVNS.

Triggering receptor expressed on myeloid cells-1 (TREM-1) amplifies the inflammatory response (Colonna et al. 2003) and is upregulated under inflammatory conditions including cancer (Wang et al. 2004). For downstream signal transduction, TREM-1 is coupled to the immunoreceptor tyrosine-based activation motif (ITAM)-containing adaptor, DNAX activation protein of 12 kDa (DAP12). TREM-1/DAP-12 receptor complex activation enhances release of multiple cytokines including monocyte chemoattractant protein-1 (MCP-1), tumor necrosis factor-α (TNFα), interleukin-1α (IL-1α), IL-1β, IL-6 and colony-stimulating factor 1 (referred to herein as CSF1; also referred to in the art as M-CSF) (Schenk et al. 2007, Lagler et al. 2009, Sigalov 2014).

Binding of CSF1 or the interleukin 34 ligand (referred to herein as IL-34) to CSF1 receptor (referred to herein as CSF1R) leads to receptor dimerization, upregulation of CSF1R protein tyrosine kinase activity, phosphorylation of CSF1R tyrosine residues, and downstream signaling events. CSF1R activation by CSF1 or IL-34 leads to the trafficking, survival, proliferation, and differentiation of monocytes and macrophages, as well as other monocytic cell lineages such as osteoclasts, dendritic cells, and microglia.

Many tumor cells or tumor stromal cells have been found to produce CSF1, which activates monocyte/macrophage cells through CSF1R. The level of CSF1 in tumors has been shown to correlate with the level of tumor-associated macrophages (TAMs) in the tumor. Higher levels of TAMs have been found to correlate with poorer patient prognoses in the majority of cancers. In addition, CSF1 has been found to promote tumor growth and progression to metastasis in, for example, human breast cancer xenografts in mice (Paulus et al. 2006). Further, CSF1R plays a role in osteolytic bone destruction in bone metastasis (Ohno et al. 2006). TAMs promote tumor growth, in part, by suppressing anti-tumor T cell effector function through the release of immunosuppressive cytokines and the expression of T cell inhibitory surface proteins. Blockade of CSF1 or CSF1R not only suppresses tumor angiogenesis and lymphangiogenesis (Kubota et al. 2009) but also improves response to T-cell checkpoint immunotherapies that target programmed cell death protein 1 (PD1) and cytotoxic T lymphocyte antigen-4 (CTLA-4) (Zhu et al. 2014). Importantly, continuous CSF1 inhibition affects pathological angiogenesis but not healthy vascular and lymphatic systems outside tumors (Kubota et al. 2009). In contrast to blockade of vascular endothelial growth factor (VEGF), interruption of CSF1 inhibition does not promote rapid vascular regrowth (Kubota et al. 2009).

Giant cell tumor of the tendon sheath (GCTTS), tenosynovial giant cell tumor (TGCT; also referred to in the art as TSGCT), and pigmented villonodular synovitis (PVNS) are the common names for a group of rare proliferative disorders that involve synovial joints and tendon sheaths. PVNS is a solid tumor of the synovium with features of both reactive inflammation and clonal neoplastic proliferation in which CSF1 is over expressed. A common translocation of the CSF1 gene (1p13) to the COL6A3 promoter (2q35) is present in approximately 60% of PVNS patients. The translocation is accompanied by CSF1 overexpression in the synovium. In addition, approximately 40% of PVNS patients have CSF1 overexpression in the absence of an identified CSF1 translocation. The consistent presence of CSF1 overexpression in all cases of PVNS and reactive synovitis suggests both an important role for CSF1 in the spectrum of synovial pathologies and the utility of targeting the CSF1/CSF1R signaling pathway therapeutically (West et al. 2006). In PVNS, CSF1 overexpression is present in a minority of synovial cells, whereas the majority of the cellular infiltrate expresses CSF1R but not CSF1. This has been characterized as a tumor-landscaping effect with aberrant CSF1 expression in the tumor cells, leading to the abnormal accumulation of non-neoplastic cells that form a mass.

Surgery is the treatment of choice for patients with localized PVNS. Recurrences occur in 8-20% of patients and are often managed by re-excision. Diffuse tenosynovial giant cell tumor (TGCT/PVNS or PVNS/dtTGCT) tends to recur more often (33-50%) and has a much more aggressive clinical course. Patients are often symptomatic and require multiple surgical procedures during their lifetime and even amputation. For patients with unresectable disease or multiple recurrences, systemic therapy using CSF1R inhibitors may help delay or avoid surgical procedures and improve functional outcomes (Radi et al. 2011).

Imatinib, a non-specific inhibitor of CSF1R, has undergone evaluation in PVNS patients (Cassier et al. 2012). Twenty-nine patients from 12 institutions in Europe, Australia, and the United States were included. The median age was 41 years and the most common site of disease was the knee (n=17; 59%). Two patients had metastatic disease to the lung and/or bone. Five of 27 evaluable patients had complete (n=1) or partial (n=4) responses per RECIST for an overall response rate of 19%. Twenty of 27 patients (74%) had stable disease. Symptomatic improvement was noted in 16 of 22 patients (73%) who were assessable for symptoms. Despite a high rate of symptomatic improvement and an overall favorable safety profile, 10 patients discontinued treatment for toxicity or other reasons.

Pexidartinib (PLX3397), a potent, selective oral CSF1R inhibitor, that traps the kinase in the autoinhibited conformation, has undergone evaluation in TGCT patients (Tap et al. 2015). A total of 41 patients were enrolled in the dose-escalation study, and an additional 23 patients were enrolled in the extension study. In the extension study, 12 patients with TGCTs had a partial response and 7 patients had stable disease. The most common adverse events included fatigue, change in hair color, nausea, dysgeusia, and periorbital edema; adverse events rarely led to discontinuation of treatment. Despite treatment of TGCTs with PLX3397 resulted in a prolonged regression in tumor volume in most patients of this Phase 2 study, later the Phase 3 study was suspended after two reported cases of nonfatal, serious liver toxicity.

Anti-CSF1R antibodies alone or in combination with antibodies against PD1 or against PDL1, one of the ligands for PD1, were proposed as less toxic alternative treatments for PVNS. See, e.g., U.S. Pat. No. 10,040,858 B2 and U.S. Pat. No. 10,221,224. As with most combination therapies, the promise of increased clinical activity is accompanied by the risk of additive toxicity and therefore requires careful assessment.

Liver enzyme elevations can be considered a class effect of CSF1R-targeting compounds (Cannarile et al. 2017). In addition, the oversuppression of the CSF1/CSF1R signaling pathway may result in potential serious long term adverse effects (AEs). In animals, CSF1 deficiency results in a range of developmental abnormalities, including skeletal, neurological, growth and fertility defects (Michaelson et al. 1996, Hume et al. 2012, Jones et al. 2013).

Thus, PVNS is a rare, locally aggressive neoplasm of the joint or tendon sheath with features of both reactive inflammation and clonal neoplastic proliferation in which CSF-1 is over expressed (Tap et al. 2015). Surgical resection is the primary treatment; however, diffuse TGCT is more difficult to resect and often involves total synovectomy, joint replacement, or amputation (Tap et al. 2015). There are no approved systemic therapies. Therefore, alternative, less toxic and more targeted treatments for PVNS are needed.

Inhibition of TREM-1 lowers levels of proinflammatory cytokines including CSF1 and is a promising approach in a variety of inflammation-associated disorders including cancer (Colonna and Facchetti 2003, Schenk et al. 2007, Pelham et al. 2014, Sigalov 2014, Shen et al. 2017, Shen et al. 2017, Rojas et al. 2018). In CD4+ T cell- and dextran sodium sulfate-induced models of colitis, Trem1−/− mice displayed significantly attenuated disease that was associated with reduced inflammatory infiltrates and diminished expression of pro-inflammatory cytokines. Trem1−/− mice also exhibited reduced neutrophilic infiltration and decreased lesion size upon infection with Leishmania major (Weber et al. 2014). Furthermore, reduced morbidity was observed for influenza virus-infected Trem1−/− mice (Weber et al. 2014). Importantly, while immune-associated pathologies were significantly reduced, Trem1−/− mice were equally capable of controlling infections with L. major, influenza virus, but also Legionella pneumophila as Trem1+/+ controls (Weber et al. 2014). Humans lacking DAP-12 do not have problems resolving infections with viruses or bacteria (Lanier 2009). Collectively, these findings suggest that in contrast to single cytokine blockers including CSF1 and CSF1R blockers, therapeutic blocking of TREM-1/DAP-12 signaling in distinct inflammatory disorders including CSF1-dependent TGCTs holds considerable promise by blunting excessive inflammation while preserving the capacity for microbial control.

The present invention provides a method of using the well-tolerable TREM-1/DAP-12 modulatory peptides and compositions for treatment of PVNS. These and other objects and advantages of the invention, as well as additional inventive features, will be apparent from the description of the invention provided herein.

Methods of treating tenosynovial giant cell tumor (TGCT) or pigmented villonodular synovitis (PVNS) with peptide variants and compositions that modulate activity of the receptor complex formed by triggering receptor expressed on myeloid cells 1 (TREM-1) and DNAX activation protein of 12 kDa (DAP12) are provided.

Inhibition of TREM-1 lowers levels of proinflammatory cytokines including CSF1 and is a promising approach in a variety of inflammation-associated disorders including cancer (Colonna and Facchetti 2003, Schenk et al. 2007, Pelham et al. 2014, Sigalov 2014, Shen et al. 2017, Shen et al. 2017, Rojas et al. 2018). In CD4+ T cell- and dextran sodium sulfate-induced models of colitis, Trem1−/− mice displayed significantly attenuated disease that was associated with reduced inflammatory infiltrates and diminished expression of pro-inflammatory cytokines. Trem1−/− mice also exhibited reduced neutrophilic infiltration and decreased lesion size upon infection with Leishmania major (Weber et al. 2014). Furthermore, reduced morbidity was observed for influenza virus-infected Trem1−/− mice (Weber et al. 2014). Importantly, while immune-associated pathologies were significantly reduced, Trem1−/− mice were equally capable of controlling infections with L. major, influenza virus, but also Legionella pneumophila as Trem1+/+ controls (Weber et al. 2014). Humans lacking DAP-12 do not have problems resolving infections with viruses or bacteria (Lanier 2009). Collectively, these findings suggest that in contrast to single cytokine blockers including CSF1 and CSF1R blockers, therapeutic blocking of TREM-1/DAP-12 signaling in distinct inflammatory disorders including CSF1-dependent TGCTs holds considerable promise by blunting excessive inflammation while preserving the capacity for microbial control.

The present invention provides a method of using the well-tolerable TREM-1/DAP-12 modulatory peptides and compositions for treatment of PVNS. These and other objects and advantages of the invention, as well as additional inventive features, will be apparent from the description of the invention provided herein.

4. Liver Cancer.

Globally, liver cancer is the fifth commonest cancer in 2012, accounting for 9.1% of all cancer deaths worldwide with the overall 5-year relative survival rate for patients with liver cancer of 17%. Owing to its extremely aggressive nature and poor survival rate, it remains an important public health issue worldwide (Wong et al. 2017)

5. Breast Cancer.

Breast cancer is the most common malignancy in women around the world (Ghoncheh et al. 2016). Ii is the most common cancer in women, accounting for 25.1% of all cancers. Breast cancer incidence in developed countries is higher, while relative mortality is greatest in less developed countries (Ghoncheh et al. 2016). Despite significant improvements in clinical outcomes within the field of breast cancer in the last 50 years, the triple-negative breast cancer (TNBC) subtype remains an area of huge unmet clinical need (Partridge et al. 2017).

6. Glioblastoma.

Glioblastoma Multiforme (GBM) is the most common and lethal type of brain cancer (Shergalis et al. 2018). For adults with GBM, treated with standard first-line therapy—concurrent radiation and temozolomide (TMZ) therapy followed by TMZ monotherapy, the median survival is about 14.6 months (Grossman et al. 2010, Shergalis et al. 2018). Little progress has been made over the past several decades in the treatment of GBM, highlighting an urgent need for new therapies.

7. Colorectal Cancer.

Colorectal cancer (CRC) has a considerable impact on patients and healthcare systems in developed countries and around 25% of patients present with metastatic disease that significantly impacts on prognosis (Van Cutsem et al. 2013). For those with localized CRC of stages I and II, the 5-year survival rate is as high as 93%, declining to 60%, 42% and 25% for patients with stages IIIA, IIIB and IIIC, respectively. However, most patients with metastatic CRC (stage IV) are not curable, with the 5-year survival rate falling to less than 10%. While early diagnosis of CRC in recent years combined with advances in treatment has considerably improved survival, management of the disease remains challenging and further progress is needed (Van Cutsem et al. 2013).

### Scleroderma, Related Autoimmune Conditions and Fibrotic Conditions.

It is estimated that scleroderma or systemic sclerosis (SSc) affects 100,000-300,000 Americans, predominantly young to middle aged women. Systemic sclerosis is a progressive and untreatable disease of unknown cause and high mortality. Fibrosis in SSc resembles uncontrolled wound healing, where healing occurs by intractable fibrosis rather than normal tissue regeneration.

It is believed that SSc is associated with the highest case-fatality rates among the rheumatic diseases or connective tissue diseases. Currently, there are no validated biomarkers for diagnosis. Furthermore, no effective disease-modifying therapies are currently available. In fact, while some treatment can alleviate the pain associated with SSc, to date no therapy has been shown to significantly alter survival. The pathogenesis of SSc is characterized by early vascular injury, with inflammation followed by progressive tissue damage and fibrosis. Excessive production of collagen and ECM and accumulation of myofibroblasts in lesional tissues are believed to be responsible for progressive organ failure. Pathological fibrosis resembles a normal wound healing response that has become deregulated. It is estimated that fibrosis accounts for >25% of all deaths in the U.S. Thus, fibrosis represents one of the major unmet medical needs.

Accordingly, there is a need for an effective anti-fibrotic therapy.

### Project Summary/Abstract

Scleroderma that includes localized scleroderma (LS) and systemic sclerosis (SSc) is a rare but devastating autoimmune disorder. Current therapies all have side effects, are limited and associate with 10 year survival of 55%, showing the need for novel approaches. The long-term goal of this project is to develop a new mechanism-based, efficient and well tolerable scleroderma therapy.

Triggering receptor expressed on myeloid cells 1 (TREM-1), an inflammation amplifier, contributes to the development of fibrosis in SSc. In patients, number of activated macrophages in the fibrotic areas is increased and associates with fibrosis severity. Activation of TREM-1 leads to overproduction of MCP-1/CCL2 and M-CSF/CSF-1, resulting in macrophage recruitment to an injured area and the sclerotic lesion formation in rats with scleroderma. In animal models, TREM-1 blockade inhibits inflammation and ameliorates a variety of autoimmune diseases. The hypothesis of the “proof-of-concept” Phase I is that TREM-1 blockade can prevent and treat scleroderma.

Current TREM-1 inhibitors all attempt to block binding of TREM-1 to its still uncertain ligand(s). To minimize risk of failure in clinical development, we developed a first-in-class ligand-independent TREM-1 inhibitory peptide GF9 that is well-tolerated and can be formulated into SignaBlok's long half-life macrophage-specific lipopeptide complexes (LPC) to improve its half-life and targeting to the inflammation areas. The major goal of the Phase I study is to show that TREM-1 blockade by GF9-LPC alleviates the disease in a bleomycin (BLM)-induced mouse model of scleroderma.

Phase I specific aims are to: 1) optimize TREM-1 inhibitory compositions for their functionality in vitro and pharmacokinetics in vivo and select the lead, 2) test two doses of the lead selected in a BLM-induced mouse model of scleroderma. We will generate, optimize and select the lead based upon its functionality in vitro and its PK profile in vivo. We will test two doses of the lead for its ability to prevent and treat lung, heart, muscle and skin fibrosis in a mouse model of multiorgan fibrosis in vivo. Histology/IHC studies will be performed. Serum and tissue cytokines will be evaluated, nonlimiting examples including MCP-1, CSF-1, VEGF, TGF-beta, TNF-alpha, IL-6, and IL-1-beta, will be analyzed.

It is anticipated that the Phase I study will identify a novel, first-in-class, well tolerable agent as a powerful platform for development of an effective and well-tolerable systemic scleroderma therapy, thereby improving treatment and survival of patients. Its anticipated safety is supported by good tolerability of SignaBlok's GF9-based formulations by long term-treated mice. Prototypes of SignaBlok's LPC are well tolerated in humans. TREM-1 blockade by SignaBlok competitor's inhibitory peptide LR12 (Inotrem, France) was safe in healthy and septic subjects. If successful, Phase I will be followed in Phase II by toxicology, ADME, pharmacology and CMC studies, filing an IND and subsequent evaluation in humans.

### Project Narrative.

Scleroderma (also known as systemic sclerosis) is a rare autoimmune disorder that affects about 20 to 24 people per million population in the US each year, with the majority being women of childbearing age. There is no approved drug for scleroderma. Current therapies all have side effects, are limited and associated with 10 year survival of 55%, highlighting the urgent need for novel approaches The proposed research is anticipated to result in the development of novel mechanism-based first-in-class therapeutics that could substantially improve treatment of scleroderma and patient survival.

### Specific Aims.

The Product. The final product will represent a new mechanism-based, efficient, stable, well tolerable systemic immunomodulatory therapy for scleroderma in order to significantly decrease long-term disability, morbidity and mortality of the patients with scleroderma and improve the quality of their life.

Scleroderma is a rare but devastating autoimmune disorder (Lawrence et al. 1998, Mayes et al. 2003, Helmick et al. 2008) with no approved drug available. Current main treatments all have side effects, are limited and associated with 10 year survival of 55% (Badea et al. 2009, Kowal-Bielecka et al. 2009, Shah et al. 2013), highlighting an urgent need for new therapies. Macrophages are associated with fibrosis (Ishikawa et al. 1992, Kraling et al. 1995, Lech et al. 2013, Chia et al. 2015) and are recruited to inflammation sites by MCP-1 which is significantly elevated in patients with systemic sclerosis (SSc) (Hasegawa et al. 1999). Activated macrophages produce VEGF, IL-1beta, TNFalpha, IL-6, TGFbeta and PDGF that play a role in scleroderma (Bonner et al. 1991, Clouthier et al. 1997, Yamamoto 2011, Yamamoto et al. 2011, Liu et al. 2013, Manetti 2015). In animals, blockers of IL-6 receptor (IL-6R) (Kitaba et al. 2012), VEGF (Koca et al. 2016), TNFalpha (Koca et al. 2008) and TGFalpha (Varga et al. 2009, Varga et al. 2009) alleviate scleroderma but all may have serious side effects including fatal infections and sepsis (Varga 2004). CSF-1/M-CSF plays a role in pulmonary fibrosis that occurs in 90% of scleroderma patients (Baran et al. 2007). TREM-1 mediates release of MCP-1/CCL2, TNFalpha, IL-1beta, IL-6 and CSF-1 (Schenk et al. 2007, Dower et al. 2008, Lagler et al. 2009, Sigalov 2014, Shen et al. 2015). TREM-1 expression is increased in the lungs of mice with BLM-induced pulmonary fibrosis (Peng et al. 2016). Together, this implicates TREM-1 as a new target to develop a first-in-class therapy for scleroderma.

Innovation. At least two aspects: 1. This is the first project to study TREM-1 blockade in an animal model of scleroderma. 2. To block TREM-1, we use a proprietary peptide GF9 formulated into macrophage-specific LipoPeptide Complexes (LPC) to extend its half-life and increase targeting (Sigalov 2014, Shen et al. 2017, Shen et al. 2017). Other TREM-1 blockers (e.g., LR12 peptide by Inotrem, France (Cuvier et al. 2018)) all attempt to block binding of currently uncertain ligands of TREM-1 and have a risk of failure in clinics, while GF9 is an advantageously ligand-independent.

Previously ((Sigalov 2014, Shen and Sigalov 2017, Shen and Sigalov 2017, Tornai et al. 2019), Preliminary Data), we found that TREM-1 blockade using GF9: ameliorates disease in mice with collagen-induced arthritis (CIA); reduces serum CSF-1, TNFalpha, IL-1alpha, IL-6 in mice with CIA, cancer, and liver disease; and inhibits expression of MCP-1/CCL2, TNFalpha, Pro-Coll1-alpha and alpha-SMA in mice with liver disease.

The goal of this project is to develop TREM-1-targeting drug for the treatment of scleroderma.

Aim 1: Optimize TREM-1 inhibitory compositions for their functionality in vitro and pharmacokinetics in vivo and select the lead. GF9-LPC will be generated using GF9, lipids and two modified peptides that mediate macrophage uptake of GF9-LPC and affect their half-life in vivo. We will vary lipid/peptide composition and peptide ratios to prepare long half-life GF9-LPC with fast and high uptake by J774 cells and high inhibitory effect on cytokine release by LPS-stimulated J774 cells. Three most promising GF9-LPC injectables selected based on their functionality in vitro will be tested in rats for their pharmacokinetic (PK) profiles. To analyze GF9 in animal serum, we will develop and validate an LC-MS assay with ZATA Pharmaceuticals. Milestone 1 includes development of the long half-life lead, which is efficient in inhibiting cytokine release in vitro. Completion of the Aim 1 will answer the question on the possibility of generating of the lead optimized to provide fast, efficient and long-lasting therapeutic effect.

Aim 2: Test two doses of the GF9-LPC lead in a bleomycin-induced mouse model of scleroderma. We have shown that chronic subcutaneous injection of BLM in mice results in the development of progressive multiple organ fibrosis with histological changes in the skin, muscle and lungs that resemble those seen in patients with SSc (Bhattacharyya et al. 2018, Bhattacharyya et al. 2018). Two doses of the GF9-LPC lead generated in the Aim 1 will be tested for its effect on lung, heart, muscle and skin fibrosis in this mouse model. Studies will be performed at Northwestern Scleroderma by lab of Dr. John Varga, a world-renowned expert in autoimmune diseases with special emphasis on scleroderma. Histology/IHC studies will be performed. Serum and tissue CCL2, CSF-1, VEGF, TGFbeta, TNFalpha, IL-6, and IL-1beta will be analyzed. Milestone 2 includes in vivo testing of suitability of TREM-1 blockade to prevent and treat scleroderma. Completion of the Aim 2 will answer a question about feasibility of using GF9-LPC as a first-in-class therapy for scleroderma.

The project is anticipated to identify the lead that will set the stage for development of first-in-class, safe and effective scleroderma therapies. If successful, Phase I will be followed in Phase II by toxicology, pharmacology ADME, PK/PD, and CMC studies, filing an IND and subsequent evaluation in humans.

Anticipated low toxicity of GF9 therapy is supported by the safety and well tolerability of 300 mg/kg GF9 in healthy mice (Sigalov 2014) (while its therapeutic dose varies from 2.5 mg/kg for GF9-LPC to 25 mg/kg for free GF9 (Sigalov 2014, Shen and Sigalov 2015, Rojas et al. 2017, Shen and Sigalov 2017, Tornai et al. 2019)) and lack of body weight changes in cancer and arthritic mice long-term treated with GF9-LPC (Sigalov 2014, Shen and Sigalov 2017). Prototypes of SignaBlok's LPC are safe in humans (Newton et al. 2002, Kingwell et al. 2013). TREM-1 blockade using peptide LR12 developed by SignaBlok's top competitor (Inotrem, France) is safe in humans (Cuvier et al. 2018, Francois et al. 2018).

Successful completion of Phase I will provide the animal proof of concept that might be applicable not only to scleroderma but also to other rare musculoskeletal, rheumatic or skin diseases.

### Research Strategy

Scleroderma: An unmet need for an effective and low toxic treatment options Scleroderma is a rare but devastating autoimmune disorder (Lawrence et al. 1998, Mayes et al. 2003, Helmick et al. 2008) with no approved drug available. Current main treatments all have side effects, are limited and associated with 10 year survival of 55% (Badea et al. 2009, Kowal-Bielecka et al. 2009, Shah and Wigley 2013), highlighting an urgent need for new therapies. The long-term goal of the proposed project is to develop a novel, first-in-class, efficient and well tolerable systemic therapy for scleroderma.

### Macrophages and Scleroderma.

Macrophages are the predominant infiltrating cells in skin lesions of patients with scleroderma and are associated with fibrosis (Ishikawa and Ishikawa 1992, Kraling et al. 1995, Lech and Anders 2013, Chia and Lu 2015). MCP-1 recruits macrophages to inflammation sites and is significantly elevated in patients with systemic sclerosis (SSc) (Hasegawa et al. 1999). Activated macrophages produce VEGF, IL-1-beta, TNFalpha, IL-6, TGF-beta and PDGF, which are of crucial importance in the profibrogenic role of fibroblasts in scleroderma (Bonner et al. 1991, Clouthier et al. 1997, Yamamoto 2011, Yamamoto and Katayama 2011, Liu et al. 2013, Manetti 2015). In animals, blockers of IL-6 receptor (IL-6R) (Kitaba et al. 2012), VEGF (Koca et al. 2016), TNF-alpha (Koca et al. 2008) and TGF-beta (Varga and Pasche 2009, Varga and Whitfield 2009) alleviate scleroderma but all may have serious side effects including fatal infections and sepsis (Varga 2004). M-CSF plays a role in pulmonary fibrosis that occurs in 90% of scleroderma patients (Baran et al. 2007). In rats, elevated MCP-1 and M-CSF lead to macrophage recruitment in an injured area and to the lesion formation (Juniantito et al. 2013).

Inhibition of TREM-1 Signaling: A New Approach to Disorders Associated with Systemic Inflammation

Triggering Receptor Expressed on Myeloid cells-1 (TREM-1), an inflammation amplifier, plays a role in immune response (Bouchon et al. 2000, Bouchon et al. 2001, Bleharski et al. 2003, Colonna et al. 2003, Klesney-Tait et al. 2006, Tessarz et al. 2008) and is upregulated upon inflammation (Wang et al. 2004, Gonzalez-Roldan et al. 2005, Koussoulas et al. 2006, Schenk et al. 2007). TREM-1 mediates release of multiple cytokines including MCP-1, TNF Q, IL-1□, IL-6 and M-CSF (Schenk et al. 2007, Dower et al. 2008, Lagler et al. 2009, Sigalov 2014, Shen and Sigalov 2015). TREM-1 blockade is a new approach to inflammatory disorders (Bouchon et al. 2001, Colonna and Facchetti 2003, Schenk et al. 2007, Gibot et al. 2008, Ho et al. 2008, Ford et al. 2009, Gibot et al. 2009, Murakami et al. 2009, Luo et al. 2010, Pelham et al. 2014, Pelham et al. 2014, Bosco et al. 2016), In mice, TREM-1 blockade inhibits M-CSF, TNFalpha, IL-1beta and IL-6, suppresses tumor growth and ameliorates autoimmune arthritis (Sigalov 2014, Shen and Sigalov 2017).

TREM-1 blockade blunts excessive inflammation but in contrast to single cytokine blockers, preserves the capacity for microbial control (Weber et al. 2014). TREM-1 blockade was suggested as a treatment of neonatal infection (Qian et al. 2014). Endotoxic and septic mice lacking DAP12, a signaling adapter of TREM-1, have improved survival (Turnbull et al. 2005). Humans lacking DAP12 do not have problems resolving infections (Lanier 2009).

Inhibition of TREM-1 signaling: A new approach to preventing and treating scleroderma TREM-1 is overexpressed in the lungs of mice with BLM-induced pulmonary fibrosis (Peng et al. 2016). In experimental autoimmune arthritis, cancer and retinopathy, TREM-1 blockade reduces inflammation and inhibits the macrophage infiltration/activation (Sigalov 2014, Shen and Sigalov 2017, Shen and Sigalov 2017) (Section 3.3.3.1). In mice with alcohol-induced liver disease (ALD), TREM-1 blockade inhibits expression of TREM-1, MCP-1/CCL2, TNF□, Pro-Coll1□ and □-SMA (Tornai et al. 2019). Collectively, these findings implicate TREM-1 as a target for development of new therapy for scleroderma.

The main concepts of the proposed project: Silencing the scleroderma-related TREM-1-specific inflammatory response can be superior to anti-single cytokine strategies in the treatment of scleroderma in terms of safety and efficacy; Delivery of systemically administered TREM-1 blockers to macrophages may have several advantages: (a) striking the target cell population, (b) sparing other cells that have no (or marginal) effects on scleroderma, (c) minimizing off-target effects, and (d) reducing the therapeutic dose; and Rate and efficiency of intracellular delivery of TREM-1 blockers to macrophages may be important to provide a prompt and effective therapeutic response during scleroderma progression.

### Innovation

### TREM-1 Blockade

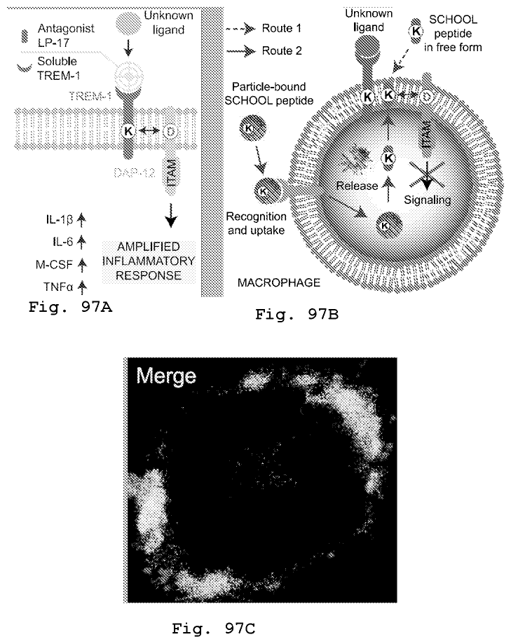

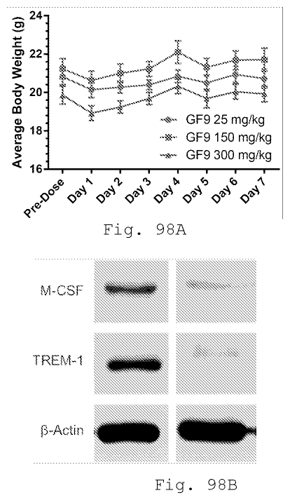

Major challenge. Current approaches (eg Inotrem's LR12) that all attempt to block TREM-1 binding to its ligand(s) (FIG. 97A) have a risk of failure since exact nature of TREM-1 ligand(s) is still uncertain (Tammaro et al. 2017).
SignaBlok's solution. Using our new model of signaling, the Signaling Chain HOmoOLigomerization (SCHOOL) model (Sigalov 2006, Sigalov 2010), we developed a first-in-class ligand-independent TREM-1 inhibitory peptide GF9 (U.S. Pat. No. 8,513,185) that disrupts recognition and signaling functions of TREM-1 in the membrane (FIG. 97B) (Sigalov 2010, Sigalov 2013, Sigalov 2014, Shen and Sigalov 2017, Shen and Sigalov 2017).

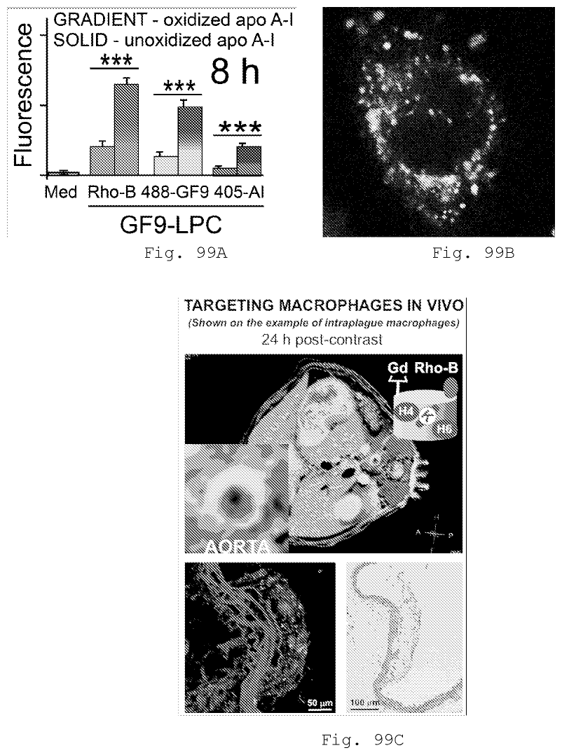

As other peptides (Graff et al. 2003, Lien et al. 2003, Gotthardt et al. 2004, Ladner et al. 2004, Prive et al. 2006, Sato et al. 2006, Antosova et al. 2009, Koskimaki et al. 2010), GF9 is advantageous compared to large protein molecules. Mechanistically, GF9 self-penetrates into the cell membrane and can reach its site of action from both inside and outside the cell (FIG. 97B and FIG. 97C). GF9 is well-tolerated by healthy mice (up to 300 mg/kg; FIG. 98A). GF9 at 25 mg/kg in a free form or at 2.5 mg/kg when formulated into LipoPeptide Complexes (LPC, below), reduces tissue TREM-1 and M-CSF upon inflammation (shown on the example of the retina of mice with oxygen-induced retinopathy, OIR) (FIG. 98B), and ameliorates diseases in mouse models of cancer (Sigalov 2014, Shen and Sigalov 2017), retinopathy (Rojas et al. 2018), ALD (Tornai et al. 2019), sepsis and autoimmune arthritis (Sigalov 2014, Shen and Sigalov 2017).

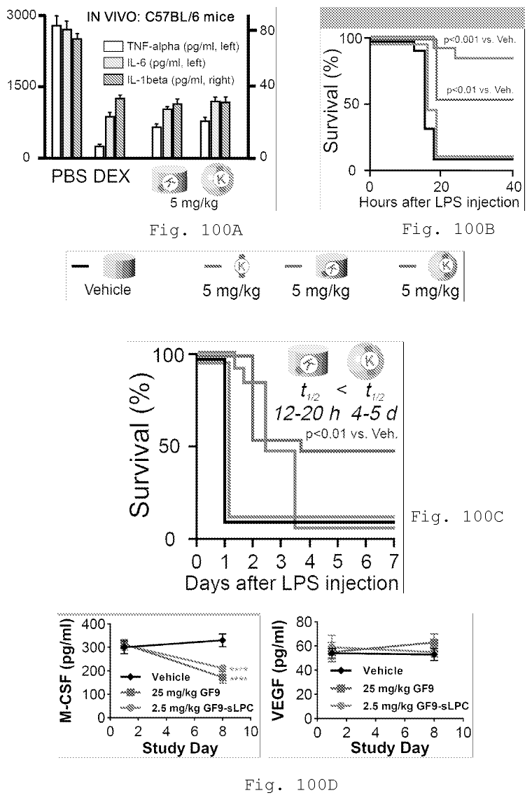

LPC mimic human High Density Lipoproteins (HDL) and consist of lipids and peptides of human apolipoprotein (apo) A-I, the major protein of HDL. In contrast to native HDL, these peptides contain naturally occurring modifications that target LPC to macrophages. SignaBlok's LPC can deliver GF9 to macrophages in vitro and in vivo (FIG. 99A-C) and increase its therapeutic efficacy (Sigalov 2014, Shen and Sigalov 2017, Shen and Sigalov 2017). NOTE: GF9-LPC describes GF9 formulated into either discoidal (GF9-dLPC, short t_{1/2}: hrs) or spherical (GF9-sLPC; long t_{1/2}: days) LPC.

Epitope-Based Rational Design of Long Half-Life GF9-LPC Fast and Effective in Delivery of GF9.

GF9-LPC tested to date, all contained a fixed amount of GF9 and an equimolar mixture of oxidized (MetSO) 22-mer peptides with sequences from either helix 4 (PE22) or 6 (PA22) of human apo A-I. Although these modifications increase macrophage uptake of LPC in vitro and in vivo (Sigalov 2014, Sigalov 2014, Shen et al. 2015) (FIG. 99A), the uptake can be optimized to make it faster and more efficient. Oxidized PE22 and PA22 contain different MetSO epitopes for binding to Scavenger Receptor (SR) SR-A (Apo A-I peptides contain putative epitopes for binding with SR-A (italics-M(O)) and SR-BI (bold). PE22: PYLDDFQKKWQEEM(O)ELYRQKVE. PA22: PLGEEM(O)RDRARAHVDALRTHLA) (Neyen et al. 2009). In addition, PA22 contains an epitope for binding to macrophage and hepatocyte SR-BI (Liadaki et al. 2000, Cai et al. 2012). Its exposure affects binding to SR-BI (de Beer et al. 2001) and can determine the LPC half life.

### Approach

### Overall Strategy, Methodology, and Analyses to be Used to Accomplish the Specific Aims

Towards the overall goal of the proposed Phase I research, we will:
Aim 1. Optimize TREM-1 Inhibitory Compositions for their Functionality In Vitro and Pharmacokinetics In Vivo and Select the Lead.
1) generate and characterize GF9-LPC of different GF9/lpid/PE22/PA22 compositions; 2) use J774 cells and the relevant antibodies to explore mechanisms of SR-mediated uptake of GF9-LPC; 3) use the mechanistic data to optimize GF9/lpid/PE22/PA22 compositions and generate long half-life GF9-LPC with high GF9 load and high rate and efficiency of macrophage delivery of GF9; 4) functionally test the generated GF9-LPC for inhibition of cytokine release in LPS-stimulated J774 cells; 5) develop an LC-MS assay for analysis of GF9 in rat serum;
6) test three most promising formulations in PK studies in Sprague-Dawley (SD) rats;
7) analyze the data obtained and select the lead GF9-LPC formulation for further animal testing.

### Aim 2: Test Two Doses of the GF9-LPC Lead in a Bleomycin-Induced Mouse Model of Scleroderma

1) test two doses of the GF9-LPC lead generated in the Aim 1 in preventative and established BLM-induced mouse models of scleroderma for its efficacy in preventing and treating the disease;
2) perform comprehensive histology/immunohistochemistry studies;
3) analyze serum and tissue GF9 and cytokines (LC-MS; ELISA).

Preliminary data Preliminary data, rationale, methodology, and analyses to be used to accomplish the Aim 1. Previously (Sigalov 2014), we showed that oxidation of PE22 and PA22 results in increased in vitro J774 cell uptake of GF9-LPC (FIG. 99A-C) and that GF9 (but not a control peptide) either in a free form (not shown) or formulated into LPC of discoidal (GF9-dLPC) or spherical (GF9-sLPC) shape inhibits cytokine release both in vitro and in vivo and protects mice from LPS-induced sepsis-related death (FIG. 100A-D). GF9-dLPC and GF9-sLPC both contained the same amount of GF9 and an equimolar mixture of oxidized PE22 and PA22.

### Rationale

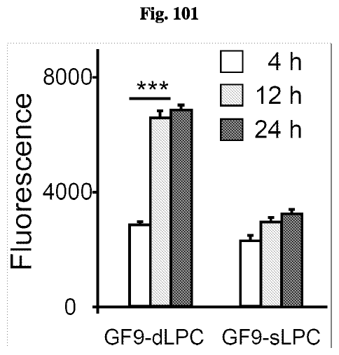

GF9-dLPC and GF9-sLPC both inhibit LPS-stimulated cytokine release in vitro and in vivo to about the same degree (FIG. 100A, FIG. 100B) but their protective effect at the dose of 5 mg/kg in LPS-induced septic mice differs: GF9-sLPC provide less effective but longer-lasting protection as compared with GF9-dLPC (FIG. 100C). Further, despite the same GF9 load and 1:1 PE22:PA22 molar ratio, these GF9-LPC differ in rate and efficiency of the macrophage uptake in vitro (FIG. 101) (Sigalov 2014). Stronger protection by GF9-dLPC may result from higher efficiency and rate of their uptake (FIG. 101), while longer protection by GF9-sLPC—from their longer half-life. Thus, uptake of GF9-LPC may depend on exposure of SR-binding apo A-I epitopes (Liu et al. 2002, Horiuchi et al. 2003) (Apo A-I peptides contain putative epitopes for binding with SR-A (italics-M(O)) and SR-BI (bold). PE22: PYLDDFQKKWQEEM(O)ELYRQKVE. PA22: PLGEEM(O)RDRARAHVDALRTHLA) that affect both rate and efficiency of the uptake.

In Phase I Aim 1, we will optimize exposure of SR-A- and SR-BI-binding epitopes and GF9 content of long half-life GF9-LPC by varying of GF9/lipid/PE22/PA22 ratios to increase GF9 load and rate and efficiency of its delivery in vivo and thus to provide prompt, effective and long-term therapeutic response.

### Methodology and Analyses

Peptides. GF9 and two oxidized 22-mer peptides PE22 and PA22 will be ordered from Bachem, Inc. and characterized as described previously (Sigalov et al. 1998, Sigalov et al. 2001, Sigalov et al. 2002, Sigalov 2014, Shen et al. 2015, Shen and Sigalov 2017, Shen and Sigalov 2017).

Long half-life GF9-LPC (spherical). Previously used non-optimized GF9-LPC be synthesized as described (Sigalov 2014, Shen and Sigalov 2017, Shen and Sigalov 2017) and used as a reference in all in vitro studies. In some studies, GF9 and/or PE22 will be Dylight (Dy) 488-labeled. In some studies, GF9-LPC will be Rhodamine B (Rho B)-labeled.

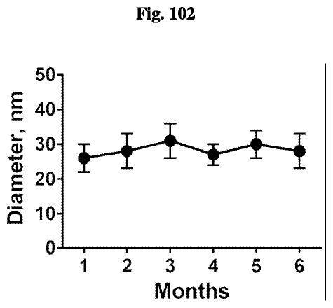

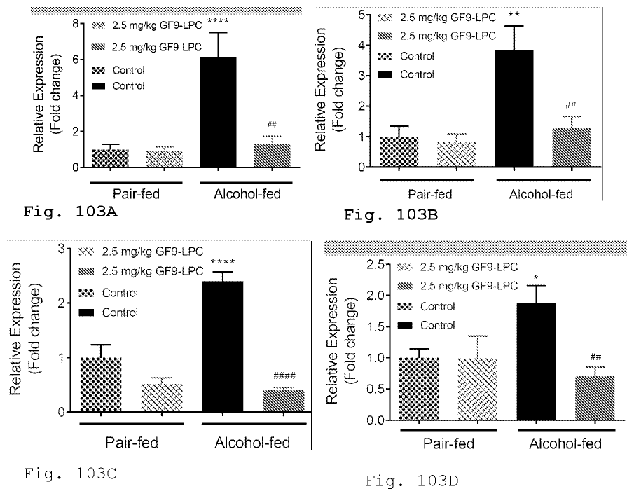

Optimization. The following parameters will be varied: a) phospholipid chain length; 2) lipid composition; 3) lipid/PE22/PA22 composition/ratio; and 4) GF9 content. The obtained GF9-LPC will be purified and their integrity, stability, and GF9 content will be analyzed as reported (Sigalov 2014, Shen and Sigalov 2017). As analyzed by Dynamic Light Scattering (DLS), GF9-LPC are stable at 4° C. for at least, up to 6 months (FIG. 102).

In vitro macrophage uptake assay. GF9-LPC will be characterized by in vitro macrophage uptake assay as reported (Shen and Sigalov 2017, Shen and Sigalov 2017, Tornai et al. 2019). To explore the mechanisms of GF9-LPC uptake, cells will be incubated with either anti-SR-BI, anti-SR-A, or isotype controls for 15 min on ice before adding Rho B-labeled GF9-LPC with Dy 488-labeled GF9 and/or PE22. After incubation, cells will be washed, lysed and fluorescence and protein concentrations in the lysates will be measured.

In vitro cytokine release. The assay will be performed in LPS-stimulated J774 macrophages (FIG. 100B) as previously reported (Sigalov 2014).

Confocal analysis. J774A.1 cells will be grown at 37° C. in 6 well tissue culture plates containing glass coverslips. After reaching target confluency of ˜50%, cells will be incubated for 6 h at 37° C. with Rho B-GF9-LPC. In subsets of experiments, Rho B-GF9-LPC that contain Dylight 488-PE22 or Dylight 488-GF9 will be used. TREM-1 staining will be performed as described (Shen and Sigalov 2017). The slides will be imaged as reported (Shen and Sigalov 2017).

Integrity and stability studies. RP-HPLC, SEC, and DLS will be used as described (Sigalov 2014, Shen and Sigalov 2017, Shen and Sigalov 2017) to study structural integrity and stability of GF9-LPC.

LC-MS for GF9 analysis in animal serum. LC-MS assay for analysis of GF9 in rat serum in PK studies in rats will be developed and validated (with ZATA). The assay will include ultracentrifugation step followed by LC-MS. The snap-frozen samples of rat serum will be ordered from BioreclamationIVT (Westbury, N.Y.) and processed as reported (Walther et al. 2011, Yanachkov et al. 2011, Yanachkova et al. 2015, Yanachkov et al. 2016). GF9-LPC will be added to serum and GF9 will be assayed by LC-MS. The assay will be validated using the FDA guidelines (https://www.fda.gov/downloads/drugs/guidances/ucm368107.pdf).

PK studies in SD rats. Animal studies will be provided by WBI. SignaBlok will perform LC-MS/histology/IHC. Sex as a biological variable. To exclude differences in PK in male and female rats (Shelnutt et al. 1999), we propose to use both sexes. 3 most promising GF9-LPC selected based on their TREM-1 inhibitory activity in vitro will be tested in 8 wk-old SD rats (200-250 g) (3 groups; 3 males+3 females/group, 18 SD rats). Briefly, SD rats will be IV administered with 2.5 mg/kg GF9-sLPC. Serum samples will be collected at 8 post-injection timepoints within 24 hrs, frozen and shipped to SignaBlok for LC-MS analysis of GF9. Ultracentrifugation of serum to float lipoproteins and GF9-LPC will be performed as reported (Sigalov et al. 1991, Sigalov 1993, Sigalov et al. 1997, Sigalov and Stem 1998, Sigalov and Stern 2001). Briefly, 50 DL serum, 50 DL saline, 0.5 mM EDTA, and 130 DL KBr (d=1.37 g/mL) will be mixed (final d=1.21 g/mL) and centrifuged in a 42.2 TI rotor at 42,000 rpm for 12 h at 10° C. 50 DL will be taken from top, dialyzed for 4 h at 4° C. and analyzed for GF9 by LC-MS.

Statistical analysis. GraphPad Prism will be used for statistical testing. In in vitro uptake assay and cytokine assay data, statistical significances will be determined by two-tailed Student's t test as described (Sigalov 2014). Results will be considered significant at p<0.05. PK data will be analyzed using PKSolver, a menu-driven add-in Microsoft Excel software (Zhang et al. 2010).

### Outcome Measures

Stability of GF9-LPC will be tested by DLS (FIG. 102). GF9 in GF9-LPC will be analyzed as reported (Sigalov 2014, Shen and Sigalov 2017) and by LC-MS. In vitro J774 cell uptake will be measured by Rho B fluorescence in cell lysates (Sigalov 2014). Activity of GF9-LPC in reduction of cytokine release by LPS-stimulated cells will be tested as reported (Sigalov 2014). In PK studies, half-life, Cmax, Tmax and the area under the AUC will be analyzed.

### Anticipated Results and Interpretations

Native dHDL and sHDL have half-lives of 12-20 hrs and 3-5 days, respectively (Scanu et al. 1962, Furman et al. 1964). We expect that formulation of GF9 into spherical LPC will extend its half-life closer to that for sHDL. Based on our preliminary data and (Sigalov 2014), we predict that: 1) GF9-sLPC of different compositions will have different exposure of SR-A and SR-BI epitopes, and 2) use of SR inhibitors will allow to find the preferential receptor involved in cell uptake. We predict that PK profiles of GF9-LPC formulations with different exposure of SR-A and SR-BI epitopes will differ. Thus, we anticipate to optimize SR-A/SR-BI epitope exposure and prepare GF9-LPC with high in vitro efficacy and favorable PK in vivo. Completion of Aim 1 will show the feasibility of using of epitope-based design to optimize GF9-LPC for effective and long-term inhibition of TREM-1 in vitro and in vivo. Milestone 1 includes selection of the lead based on its stability, in vitro activity and PK profile.

### Anticipated Problems, Alternative Strategies and Future Directions.

We do not expect technical problems as we at SignaBlok, Drs. Tabatadze and Yanachkov at ZATA, and the WBI's staff have expertise in all methods (Yanachkov et al. 2011, Sigalov 2014, Sigalov 2014, Shen et al. 2015, Yanachkova et al. 2015, Shen et al. 2016, Yanachkov et al. 2016, Shen and Sigalov 2017, Shen and Sigalov 2017, Yanachkov et al. 2017).

### Preliminary Data

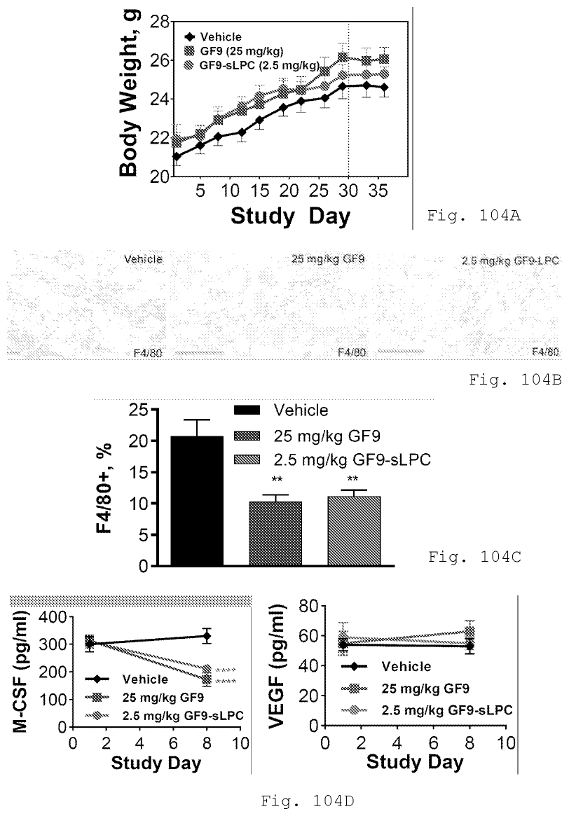

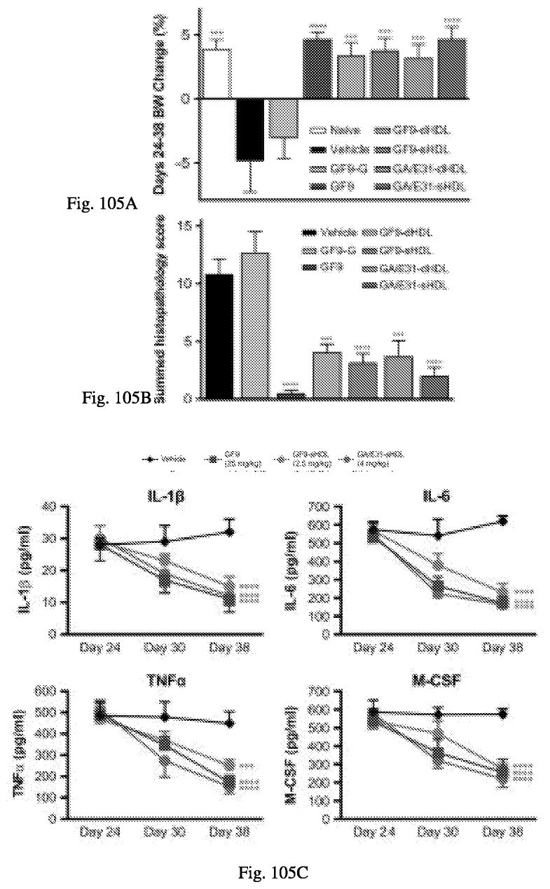

Previously, using non-optimized GF9-LPC, we demonstrated that:
1) in mice with ALD, systemic 2.5 mg/kg GF9-LPC reduces TREM-1, MCP-1/CCL2, early fibrosis markers (alpha-smooth muscle actin [alpha-SMA] and procollagen1-alpha [Pro-Coll1-alpha]) at the mRNA level (Tornai et al. 2019) FIG. 103A-D);
2) in cancer mice, systemic 25 mg/kg GF9 and 2.5 mg/kg GF9-LPC are well-tolerated (FIG. 104A), reduce macrophage infiltration into the tumor (FIG. 103B, FIG. 103C) and inhibit CSF-1/M-CSF (FIG. 104D) (Shen and Sigalov 2017);
3) in mice with CIA, systemic 25 mg/kg GF9 and 2.5 mg/kg GF9-LPC are well-tolerated (FIG. 105AA), ameliorate arthritis (FIG. 104B) and inhibit IL-1-beta, IL-6, TNF-alpha and CSF-1/M-CSF (FIG. 105AC) (Shen and Sigalov 2017).

### Rationale

TREM-1 blockade by GF9-LPC suppress macrophage infiltration and activation, reduce cytokine, CSF-1/M-CSF and early fibrosis markers and ameliorate disease in ALD, cancer, septic and arthritic mice ((Sigalov 2014, Shen and Sigalov 2017, Shen and Sigalov 2017, Tornai et al. 2019), FIG. 103A-D-FIG. 105A-C). This suggests that GF9-LPC will be effective in the treatment of scleroderma (Ishikawa and Ishikawa 1992, Kraling et al. 1995, Lech and Anders 2013, Chia and Lu 2015). BLM mouse model is a valuable tool for drug development for scleroderma (Yamamoto et al. 1999, Yamamoto et al. 1999, Huber et al. 2007, Beyer et al. 2010, Kitaba et al. 2012, Avci et al. 2013, Artlett 2014, Toyama et al. 2016). We have shown that chronic subcutaneous (s.c.) injection of BLM in mice results in development of progressive multiple organ fibrosis (Bhattacharyya et al. 2018, Bhattacharyya et al. 2018). In Aim 2, we will use this model to test GF9-LPC ability to prevent and treat organ fibrosis. Serum and tissue CCL2, CSF-1/M-CSF, VEGF, TGF Q, TNF Q, IL-6, and IL-1□ will be analyzed.

### Methodology and Analyses

We will design perform and analyze animal studies with Dr. John Varga, M.D. (Director, Northwestern Scleroderma, Northwestern University Feinberg School of Medicine (Chicago, Ill.).

Sex (gender) as a biological variable. In a BLM-induced scleroderma mouse model, while a more pronounced fibrosis phenotype was reported for male compared with female mice (Ruzehaji et al. 2015), other data show no histologic differences between male and female mice (Yamamoto et al. 1999, Yamamoto et al. 1999) We suggest to use both sexes of mice in this project.

Mouse model of scleroderma. S.c. BLM delivery leads to slowly-progressive fibrosis in multiple organs with no mortality, and histological changes in the skin, muscle and lungs that resemble those seen in patients with SSc (Bhattacharyya et al. 2018, Bhattacharyya et al. 2018). 8-12 wk-old C57BL6 mice (288 in total) will be randomized and divided into 3 arms by 12 groups of 8 mice per group (6 male and 6 female groups). In preventative model (arms 1 and 2), mice will receive s.c. injections of 10 mg/kg BLM or PBS daily for 10 days (5 days/week), along with 2.5 or 5 mg/kg GF9-LPC by daily i.p. injections starting concurrently with BLM, and will be sacrificed on day 7 (arm 1) or 22 (arm 2). In established model (arm 3), mice will receive 2.5 or 5 mg/kg GF9-LPC daily starting at day 15, and continue until sacrifice at day 28. In all arms, control groups of mice will receive BLM or PBS alone daily for 10 (7, arm 1) days or 2.5 or 5 mg/kg GF9-LPC alone daily until sacrifice at days 7, 22 or 28.

Statistical analysis. Statistical significance of differences in parameters of fibrosis and inflammation between control and treated mice will be determined by F-test. Comparison among three or more groups will be executed with one-way ANOVA followed by a post hoc Tukey's test. Based on our previous studies using this model (Bhattacharyya et al. 2018, Bhattacharyya et al. 2018), a sample size of 8 mice in each group is chosen to give a power of 80% to detect 10% difference in mean values between experimental and control groups, with a significance level of 0.05.

### Outcome Measures

Experiments will test efficacy of treatment given as prevention, as well as treatment, to determine if TREM-1 inhibition can promote regression of established skin, lung and heart fibrosis and resolution of tissue damage. Clinical observations (daily) and body weights (weekly) will be made until termination. DRAIZE scoring will be recorded once weekly for all groups. Effect of TREM-1 blockade will be tested on early (day 3-7) inflammatory changes and monocyte/macrophage influx in the lungs and skin by IHC; subsequent development of fibrotic parenchymal changes (at day 10-20) by histology/IHC, biochemical and functional assays. Tissues will be collected, prepared, stained with H&E and Trichrome and evaluated by board-certified pathologist. Part of collected tissues will be homogenized and along with blood and FFPE tissue samples shipped to SignaBlok for GF9, cytokine and IHC analysis. Tissue collagen content will be determined by hydroxyproline assays (Bhattacharyya et al. 2016). Lung fibrosis will be quantitated in histological lung sections using the modified Ashcroft score determined from 5 h.p.f. per mice (Hubner score) (Hubner et al. 2008). Skin hardness will be measured using a Vesmeter three times at the injection area. Dermal thickness will be determined at three randomly selected sites in each animal. a-SMA-positive cells will be counted. Macrophage infiltration will be evaluated by IHC. Serum and tissue CCL2, VEGF, CSF-1, TGF□, TNF□, IL-6 and IL-1□ will be analyzed by ELISA. Tissue TREM-1 expression will be tested by Western Blot.

### Anticipated Results and Interpretations

These studies are expected to demonstrate if TREM-1 blockade using GF9-LPC can, by attenuating TLR4 activity in target organs, prevent, slow the progression, and promote the recovery from, fibrotic injury in the skin, lungs, muscle and heart. Further, the results are expected to indicate whether observed beneficial effects are primarily due to attenuated early inflammation, reduced fibrosis due to attenuated activation of (myo)fibroblasts, or a combination of both of these mechanisms. Based on our previous data ((Sigalov 2014, Shen and Sigalov 2017, Shen and Sigalov 2017); FIG. 100A-D, FIG. 103A-D-FIG. 105A-C), we predict that treatment with GF9-LPC will be well-tolerated and associated with reductions in levels of CCL2, CSF-1, TNF-beta, TGF-alpha, IL-6 and IL-1beta. We expect that GF9-LPC will be effective in a dose-dependent manner and that LPC (no GF9) will be without effect. Completion of Aim 2 will answer a question about the feasibility of using GF9-LPC as a first-in-class therapy for scleroderma.

### Anticipated Problems, Alternative Strategies and Future Directions.

We do not expect technical problems as we at SignaBlok and the Varga laboratory's and animal facility' staff have extensive expertise in all methods (Varga and Whitfield 2009, Sigalov 2014, Shen et al. 2015, Shen and Sigalov 2016, Shen and Sigalov 2017, Shen and Sigalov 2017, Bhattacharyya et al. 2018, Bhattacharyya et al. 2018, Yamashita et al. 2018, Lakota et al. 2019, Tornai et al. 2019).
Final product. SignaBlok's GF9-LPC consist of phospholipids widely used in pharmacology and synthetic peptides, all derived from human sequences, suggesting the lack of potential immunogenicity. Lipoprotein- and peptide-based drug formulations are currently on the market (Chang et al. 2012, Adler-Moore et al. 2016) or in clinical trials (Tricoci et al. 2015), which makes SignaBlok's efficient and well tolerable systemic therapy for scleroderma commercially viable.

Future directions. If successful, Phase I will be followed in Phase II where to evaluate the efficacy of TREM-1 blockade for mitigating organ fibrosis, we will use complementary mouse models of SSc, including the Tsk1/+ mouse, which (spontaneously) develop skin fibrosis in the absence of inflammation. Other administration schedules and regimen will be tested. The lead and its manufacturing technology will be further optimized and the more detailed safety, TOX, ADME, CMC and other IND-enabling studies will be performed. Upon completion, an IND will be filed for subsequent testing in humans.

Anticipated low toxicity of GF9-LPC is supported by the safety of 300 mg/kg GF9 in healthy mice (Sigalov 2014) (therapeutic doses are 25 mg/kg for GF9 or 2.5 mg/kg for GF9-LPC), lack of body weight changes in mice long-term treated with GF9-LPC (Sigalov 2014, Shen et al. 2017, Tornai et al. 2019), and by the fact that prototypes of SignaBlok's LPC were well tolerated in humans (Newton and Krause 2002, Kingwell and Chapman 2013). TREM-1 blockade using inhibitory peptide LR12 which is in development by SignaBlok's top competitor (Inotrem, France) was well tolerated in healthy and septic subjects (Cuvier et al. 2018, Francois et al. 2018).

The decision to go to Phase II will be made if the significant (more than 50%) decrease in fibrosis is shown in treated mice as compared with those shown in control mice.

### BIBLIOGRAPHY AND REFERENCES CITED

1. Helmick C G, Felson D T, Lawrence R C, Gabriel S, Hirsch R, Kwoh C K, et al. Estimates of the prevalence of arthritis and other rheumatic conditions in the United States. Part I. Arthritis Rheum 2008; 58:15-25. PMID: 18163481.
2. Lawrence R C, Helmick C G, Arnett F C, Deyo R A, Felson D T, Giannini E H, et al. Estimates of the prevalence of arthritis and selected musculoskeletal disorders in the United States. Arthritis Rheum 1998; 41:778-99. PMID: 9588729.
3. Mayes M D, Lacey J V, Jr., Beebe-Dimmer J, Gillespie B W, Cooper B, Laing T J, et al. Prevalence, incidence, survival, and disease characteristics of systemic sclerosis in a large US population. Arthritis Rheum 2003; 48:2246-55. PMID: 12905479.
4. Kowal-Bielecka O, Landewe R, Avouac J, Chwiesko S, Miniati I, Czirjak L, et al. EULAR recommendations for the treatment of systemic sclerosis: a report from the EULAR Scleroderma Trials and Research group (EUSTAR). Ann Rheum Dis 2009; 68:620-8. PMID: 19147617.
5. Badea I, Taylor M, Rosenberg A, Foldvari M. Pathogenesis and therapeutic approaches for improved topical treatment in localized scleroderma and systemic sclerosis. Rheumatology (Oxford) 2009; 48:213-21. PMID: 19022832.
6. Shah A A, Wigley F M. My approach to the treatment of scleroderma. Mayo Clin Proc 2013; 88:377-93. PMID: 23541012.
7. Ishikawa O, Ishikawa H. Macrophage infiltration in the skin of patients with systemic sclerosis. J Rheumatol 1992; 19:1202-6. PMID: 1404154.
8. Chia J J, Lu T T. Update on macrophages and innate immunity in scleroderma. Curr Opin Rheumatol 2015; 27:530-6. PMID: 26352734.
9. Lech M, Anders H J. Macrophages and fibrosis: How resident and infiltrating mononuclear phagocytes orchestrate all phases of tissue injury and repair. Biochim Biophys Acta 2013; 1832:989-97. PMID: 23246690.
10. Kraling B M, Maul G G, Jimenez S A. Mononuclear cellular infiltrates in clinically involved skin from patients with systemic sclerosis of recent onset predominantly consist of monocytes/macrophages. Pathobiology 1995; 63:48-56. PMID: 7546275.
11. Hasegawa M, Sato S, Takehara K. Augmented production of chemokines (monocyte chemotactic protein-1 (MCP-1), macrophage inflammatory protein-1alpha (MIP-1alpha) and MIP-1beta) in patients with systemic sclerosis: MCP-1 and MIP-1alpha may be involved in the development of pulmonary fibrosis. Clin Exp Immunol 1999; 117:159-65. PMID: 10403930.
12. Liu J, Copland D A, Horie S, Wu W K, Chen M, Xu Y, et al. Myeloid cells expressing VEGF and arginase-1 following uptake of damaged retinal pigment epithelium suggests potential mechanism that drives the onset of choroidal angiogenesis in mice. PLoS One 2013; 8:e72935. PMID: 23977372.
13. Yamamoto T, Katayama I. Vascular changes in bleomycin-induced scleroderma. Int J Rheumatol 2011; 2011:270938. PMID: 22028717.
14. Yamamoto T. Autoimmune mechanisms of scleroderma and a role of oxidative stress. Self Nonself 2011; 2:4-10. PMID: 21776329.
15. Clouthier D E, Comerford S A, Hammer R E. Hepatic fibrosis, glomerulosclerosis, and a lipodystrophy-like syndrome in PEPCK-TGF-beta1 transgenic mice. J Clin Invest 1997; 100:2697-713. PMID: 9389733.
16. Bonner J C, Osornio-Vargas A R, Badgett A, Brody A R. Differential proliferation of rat lung fibroblasts induced by the platelet-derived growth factor-AA, -AB, and -BB isoforms secreted by rat alveolar macrophages. Am J Respir Cell Mol Biol 1991; 5:539-47. PMID: 1958381.
17. Manetti M. Deciphering the alternatively activated (M2) phenotype of macrophages in scleroderma. Exp Dermatol 2015; 24:576-8. PMID: 25869115.
18. Kitaba S, Murota H, Terao M, Azukizawa H, Terabe F, Shima Y, et al. Blockade of interleukin-6 receptor alleviates disease in mouse model of scleroderma. Am J Pathol 2012; 180:165-76. PMID: 22062222.
19. Koca S S, Ozgen M, Dagli A F, Gozel N, Ozercan I H, Isik A. The Protective Effects of Bevacizumab in Bleomycin-Induced Experimental Scleroderma. Adv Clin Exp Med 2016; 25:249-53. PMID: 27627557.
20. Koca S S, Isik A, Ozercan I H, Ustundag B, Evren B, Metin K. Effectiveness of etanercept in bleomycin-induced experimental scleroderma. Rheumatology (Oxford) 2008; 47:172-5. PMID: 18174229.
21. Varga J, Whitfield M L. Transforming growth factor-beta in systemic sclerosis (scleroderma). Front Biosci (Schol Ed) 2009; 1:226-35. PMID: 19482698.
22. Varga J, Pasche B. Transforming growth factor beta as a therapeutic target in systemic sclerosis. Nat Rev Rheumatol 2009; 5:200-6. PMID: 19337284.
23. Varga J. Antifibrotic therapy in scleroderma: extracellular or intracellular targeting of activated fibroblasts? Curr Rheumatol Rep 2004; 6:164-70. PMID: 15016348.
24. Baran C P, Opalek J M, McMaken S, Newland C A, O'Brien J M, Jr., Hunter M G, et al. Important roles for macrophage colony-stimulating factor, C C chemokine ligand 2, and mononuclear phagocytes in the pathogenesis of pulmonary fibrosis. Am J Respir Crit Care Med 2007; 176:78-89. PMID: 17431224.
25. Lagler H, Sharif O, Haslinger I, Matt U, Stich K, Furtner T, et al. TREM-1 activation alters the dynamics of pulmonary IRAK-M expression in vivo and improves host defense during pneumococcal pneumonia. J Immunol 2009; 183:2027-36. PMID: 19596984.
26. Dower K, Ellis D K, Saraf K, Jelinsky S A, Lin L L. Innate immune responses to TREM-1 activation: overlap, divergence, and positive and negative cross-talk with bacterial lipopolysaccharide. J Immunol 2008; 180:3520-34. PMID: 18292579.
27. Schenk M, Bouchon A, Seibold F, Mueller C. TREM-1-expressing intestinal macrophages crucially amplify chronic inflammation in experimental colitis and inflammatory bowel diseases. J Clin Invest 2007; 117:3097-106. PMID: 17853946.
28. Shen Z T, Sigalov A B. Novel Ligand-Independent Peptide Inhibitors of Triggering Receptor Expressed on Myeloid Cells 1 (TREM-1) and T Cell Receptor (TCR): Efficacy in a Collagen-Induced Arthritis Model Suggests New Targeted Treatment for Rheumatoid Arthritis [abstract]. Arthritis Rheumatol 2015; 67:1347-8. PMID.
29. Sigalov A B. A novel ligand-independent peptide inhibitor of TREM-1 suppresses tumor growth in human lung cancer xenografts and prolongs survival of mice with lipopolysaccharide-induced septic shock. Int Immunopharmacol 2014; 21:208-19. PMID: 24836682.
30. Peng L, Zhou Y, Dong L, Chen R Q, Sun G Y, Liu T, et al. TGF-beta1 Upregulates the Expression of Triggering Receptor Expressed on Myeloid Cells 1 in Murine Lungs. Sci Rep 2016; 6:18946. PMID: 26738569.
31. Shen Z T, Sigalov A B. Rationally designed ligand-independent peptide inhibitors of TREM-1 ameliorate collagen-induced arthritis. J Cell Mol Med 2017; 21:2524-34. PMID: 28382703.
32. Shen Z T, Sigalov A B. Novel TREM-1 Inhibitors Attenuate Tumor Growth and Prolong Survival in Experimental Pancreatic Cancer. Mol Pharm 2017; 14:4572-82. PMID: 29095622.
33. Sigalov A B. A novel ligand-independent peptide inhibitor of TREM-1 suppresses tumor growth in human lung cancer xenografts and prolongs survival of mice with lipopolysaccharide-induced septic shock. Int Immunopharmacol 2014; 21:208-19. PMID: 24836682.
34. Cuvier V, Lorch U, Witte S, Olivier A, Gibot S, Delor I, et al. A first-in-man safety and pharmacokinetics study of nangibotide, a new modulator of innate immune response through TREM-1 receptor inhibition. Br J Clin Pharmacol 2018; 84:2270-9. PMID: 29885068.
35. Tornai D, Furi I, Shen Z T, Sigalov A B, Coban S, Szabo G. Inhibition of Triggering Receptor Expressed on Myeloid Cells 1 Ameliorates Inflammation and Macrophage and Neutrophil Activation in Alcoholic Liver Disease in Mice. Hepatol Commun 2019; 3:99-115. PMID: 30619998.
36. Bhattacharyya S, Wang W, Tamaki Z, Shi B, Yeldandi A, Tsukimi Y, et al. Pharmacological Inhibition of Toll-Like Receptor-4 Signaling by TAK242 Prevents and Induces Regression of Experimental Organ Fibrosis. Front Immunol 2018; 9:2434. PMID: 30405628.
37. Bhattacharyya S, Wang W, Qin W, Cheng K, Coulup S, Chavez S, et al. TLR4-dependent fibroblast activation drives persistent organ fibrosis in skin and lung. JCI Insight 2018; 3. PMID: 29997297.
38. Rojas M A, Caldwell R B, Shen Z, Sigalov A. TREM-1 blockade prevents vitreoretinal neovascularization in a mouse model of retinopathy of prematurity [abstract]. Invest Ophthalmol Vis Sci 2017; 58:3452. PMID.
39. Kingwell B A, Chapman M J. Future of high-density lipoprotein infusion therapies: potential for clinical management of vascular disease. Circulation 2013; 128:1112-21. PMID: 24002713.
40. Newton R S, Krause B R. HDL therapy for the acute treatment of atherosclerosis. Atheroscler Suppl 2002; 3:31-8. PMID: 12573361.
41. Francois B, Wittebole X, Mira J P, Dugernier T, Gibot S, Derive M, et al. P1 Safety and pharmacodynamic activity of a novel TREM-1 pathway inhibitory peptide in septic shock patients: phase IIa clinical trial results. Intensive Care Med Exp 2018; 6(Suppl 1). PMID.
42. Juniantito V, Izawa T, Yuasa T, Ichikawa C, Yano R, Kuwamura M, et al. Immunophenotypical characterization of macrophages in rat bleomycin-induced scleroderma. Vet Pathol 2013; 50:76-85. PMID: 22700848.
43. Bouchon A, Dietrich J, Colonna M. Cutting edge: inflammatory responses can be triggered by TREM-1, a novel receptor expressed on neutrophils and monocytes. J Immunol 2000; 164:4991-5. PMID: 10799849.
44. Bouchon A, Facchetti F, Weigand M A, Colonna M. TREM-1 amplifies inflammation and is a crucial mediator of septic shock. Nature 2001; 410:1103-7. PMID: 11323674.
45. Bleharski J R, Kiessler V, Buonsanti C, Sieling P A, Stenger S, Colonna M, et al. A role for triggering receptor expressed on myeloid cells-1 in host defense during the early-induced and adaptive phases of the immune response. J Immunol 2003; 170:3812-8. PMID: 12646648.
46. Tessarz A S, Cerwenka A. The TREM-1/DAP12 pathway. Immunol Lett 2008; 116:111-6. PMID: 18192027.
47. Klesney-Tait J, Turnbull I R, Colonna M. The TREM receptor family and signal integration. Nat Immunol 2006; 7:1266-73. PMID: 17110943.
48. Colonna M, Facchetti F. TREM-1 (triggering receptor expressed on myeloid cells): a new player in acute inflammatory responses. J Infect Dis 2003; 187 Suppl 2:S397-401. PMID: 12792857.
49. Gonzalez-Roldan N, Ferat-Osorio E, Aduna-Vicente R, Wong-Baeza I, Esquivel-Callejas N, Astudillo-de la Vega H, et al. Expression of triggering receptor on myeloid cell 1 and histocompatibility complex molecules in sepsis and major abdominal surgery. World J Gastroenterol 2005; 11:7473-9. PMID: 16437719.
50. Koussoulas V, Vassiliou S, Demonakou M, Tassias G, Giamarellos-Bourboulis E J, Mouktaroudi M, et al. Soluble triggering receptor expressed on myeloid cells (sTREM-1): a new mediator involved in the pathogenesis of peptic ulcer disease. Eur J Gastroenterol Hepatol 2006; 18:375-9. PMID: 16538108.
51. Wang D Y, Qin R Y, Liu Z R, Gupta M K, Chang Q. Expression of TREM-1 mRNA in acute pancreatitis. World J Gastroenterol 2004; 10:2744-6. PMID: 15309732.
52. Gibot S, Massin F, Alauzet C, Montemont C, Lozniewski A, Bollaert P E, et al. Effects of the TREM-1 pathway modulation during mesenteric ischemia-reperfusion in rats. Crit Care Med 2008; 36:504-10. PMID: 18091551.
53. Luo L, Zhou Q, Chen X J, Qin S M, Ma W L, Shi H Z. Effects of the TREM-1 pathway modulation during empyema in rats. Chin Med J (Engl) 2010; 123:1561-5. PMID: 20819512.
54. Murakami Y, Akahoshi T, Aoki N, Toyomoto M, Miyasaka N, Kohsaka H. Intervention of an inflammation amplifier, triggering receptor expressed on myeloid cells 1, for treatment of autoimmune arthritis. Arthritis Rheum 2009; 60:1615-23. PMID: 19479878.
55. Gibot S, Massin F, Alauzet C, Derive M, Montemont C, Collin S, et al. Effects of the TREM 1 pathway modulation during hemorrhagic shock in rats. Shock 2009; 32:633-7. PMID: 19333144.
56. Ho C C, Liao W Y, Wang C Y, Lu Y H, Huang H Y, Chen H Y, et al. TREM-1 expression in tumor-associated macrophages and clinical outcome in lung cancer. Am J Respir Crit Care Med 2008; 177:763-70. PMID: 18096709.
57. Bosco M C, Raggi F, Varesio L. Therapeutic Potential of Targeting TREM-1 in Inflammatory Diseases and Cancer. Curr Pharm Des 2016; 22:6209-33. PMID: 27568730.
58. Ford J W, McVicar D W. TREM and TREM-like receptors in inflammation and disease. Curr Opin Immunol 2009; 21:38-46. PMID: 19230638.
59. Pelham C J, Agrawal D K. Emerging roles for triggering receptor expressed on myeloid cells receptor family signaling in inflammatory diseases. Expert Rev Clin Immunol 2014; 10:243-56. PMID: 24325404.
60. Pelham C J, Pandya A N, Agrawal D K. Triggering receptor expressed on myeloid cells receptor family modulators: a patent review. Expert Opin Ther Pat 2014; 24:1383-95. PMID: 25363248.
61. Weber B, Schuster S, Zysset D, Rihs S, Dickgreber N, Schurch C, et al. TREM-1 Deficiency Can Attenuate Disease Severity without Affecting Pathogen Clearance. PLoS Pathog 2014; 10:e1003900. PMID: 24453980.
62. Qian L, Weng X W, Chen W, Sun C H, Wu J. TREM-1 as a potential therapeutic target in neonatal sepsis. Int J Clin Exp Med 2014; 7:1650-8. PMID: 25126161.
63. Turnbull I R, McDunn J E, Takai T, Townsend R R, Cobb J P, Colonna M. DAP12 (KARAP) amplifies inflammation and increases mortality from endotoxemia and septic peritonitis. J Exp Med 2005; 202:363-9. PMID: 16061725.
64. Lanier L L. DAP10- and DAP12-associated receptors in innate immunity. Immunol Rev 2009; 227:150-60. PMID: 19120482.
65. Derive M, Boufenzer A, Bouazza Y, Groubatch F, Alauzet C, Barraud D, et al. Effects of a TREM-like transcript 1-derived peptide during hypodynamic septic shock in pigs. Shock 2013; 39:176-82. PMID: 23324887.
66. Derive M, Boufenzer A, Gibot S. Attenuation of responses to endotoxin by the triggering receptor expressed on myeloid cells-1 inhibitor LR12 in nonhuman primate. Anesthesiology 2014; 120:935-42. PMID: 24270127.
67. Tammaro A, Derive M, Gibot S, Leemans J C, Florquin S, Dessing M C. TREM-1 and its potential ligands in non-infectious diseases: from biology to clinical perspectives. Pharmacol Ther 2017; 177:81-95. PMID: 28245991.
68. Sigalov A B. Immune cell signaling: a novel mechanistic model reveals new therapeutic targets. Trends Pharmacol Sci 2006; 27:518-24. PMID: 16908074.
69. Sigalov A B. The SCHOOL of nature. III. From mechanistic understanding to novel therapies. Self/Nonself—Immune Recognition and Signaling 2010; 1:192-224. PMID.
70. Sigalov A B. New therapeutic strategies targeting transmembrane signal transduction in the immune system. Cell Adh Migr 2010; 4:255-67. PMID: 20519929.
71. Sigalov A B. Inhibition of TREM receptor signaling with peptide variants. U.S. Pat. No. 8,513,185. In: USPTO, ed. USA: SignaBlok, Inc., Shrewsbury, Mass. (US), 2013.
72. Ladner R C, Sato A K, Gorzelany J, de Souza M. Phage display-derived peptides as therapeutic alternatives to antibodies. Drug Discov Today 2004; 9:525-9. PMID: 15183160.
73. Antosova Z, Mackova M, Kral V, Macek T. Therapeutic application of peptides and proteins: parenteral forever? Trends Biotechnol 2009; 27:628-35. PMID: 19766335.
74. Gotthardt M, Boermann O C, Behr T M, Behe M P, Oyen W J. Development and clinical application of peptide-based radiopharmaceuticals. Curr Pharm Des 2004; 10:2951-63. PMID: 15379661.
75. Koskimaki J E, Karagiannis E D, Tang B C, Hammers H, Watkins D N, Pili R, et al. Pentastatin-1, a collagen I V derived 20-mer peptide, suppresses tumor growth in a small cell lung cancer xenograft model. BMC Cancer 2010; 10:29. PMID: 20122172.
76. Lien S, Lowman H B. Therapeutic peptides. Trends Biotechnol 2003; 21:556-62. PMID: 14624865.
77. Prive G G, Melnick A. Specific peptides for the therapeutic targeting of oncogenes. Curr Opin Genet Dev 2006; 16:71-7. PMID: 16377176.
78. Sato A K, Viswanathan M, Kent R B, Wood C R. Therapeutic peptides: technological advances driving peptides into development. Curr Opin Biotechnol 2006; 17:638-42. PMID: 17049837.
79. Graff C P, Wittrup K D. Theoretical analysis of antibody targeting of tumor spheroids: importance of dosage for penetration, and affinity for retention. Cancer Res 2003; 63:1288-96. PMID: 12649189.
80. Rojas M A, Shen Z T, Caldwell R B, Sigalov A B. Blockade of TREM-1 prevents vitreoretinal neovascularization in mice with oxygen-induced retinopathy. Biochim Biophys Acta 2018; 1864:2761-8. PMID: 29730341.
81. Sigalov A B. Nature-inspired nanoformulations for contrast-enhanced in vivo M R imaging of macrophages. Contrast Media Mol Imaging 2014; 9:372-82. PMID: 24729189.
82. Sigalov A B. Methods and Compositions for Targeted Imaging. US 20130045161. Publication Date: Feb. 21, 2013.
83. Sigalov A B. Methods and Compositions for Targeted Delivery. US 20110256224. Publication Date: Oct. 20, 2011. PMID.
84. Shen Z T, Zheng S, Gounis M J, Sigalov A B. Diagnostic Magnetic Resonance Imaging of Atherosclerosis in Apolipoprotein E Knockout Mouse Model Using Macrophage-Targeted Gadolinium-Containing Synthetic Lipopeptide Nanoparticles. PLoS One 2015; 10:e0143453. PMID: 26569115.
85. Neyen C, Pluddemann A, Roversi P, Thomas B, Cai L, van der Westhuyzen D R, et al. Macrophage scavenger receptor A mediates adhesion to apolipoproteins A-I and E. Biochemistry 2009; 48:11858-71. PMID: 19911804.
86. Liadaki K N, Liu T, Xu S, Ishida B Y, Duchateaux P N, Krieger J P, et al. Binding of high density lipoprotein (HDL) and discoidal reconstituted HDL to the HDL receptor scavenger receptor class B type I. Effect of lipid association and APOA-I mutations on receptor binding. J Biol Chem 2000; 275:21262-71. PMID: 10801839.
87. Cai L, Wang Z, Meyer J M, Ji A, van der Westhuyzen D R. Macrophage SR-BI regulates LPS-induced pro-inflammatory signaling in mice and isolated macrophages. J Lipid Res 2012; 53:1472-81. PMID: 22589557.
88. de Beer M C, Durbin D M, Cai L, Jonas A, de Beer F C, van der Westhuyzen D R. Apolipoprotein A-I conformation markedly influences HDL interaction with scavenger receptor B I. J Lipid Res 2001; 42:309-13. PMID: 11181762.
89. Horiuchi S, Sakamoto Y, Sakai M. Scavenger receptors for oxidized and glycated proteins. Amino Acids 2003; 25:283-92. PMID: 14661091.
90. Liu T, Krieger M, Kan H Y, Zannis V I. The effects of mutations in helices 4 and 6 of ApoA-I on scavenger receptor class B type I (SR-BI)-mediated cholesterol efflux suggest that formation of a productive complex between reconstituted high density lipoprotein and SR—B I is required for efficient lipid transport. J Biol Chem 2002; 277:21576-84. PMID: 11882653.
91. Sigalov A B, Stern U. Enzymatic repair of oxidative damage to human apolipoprotein A-I. FEBS Lett 1998; 433:196-200. PMID: 9744793.
92. Sigalov A B, Stern U. Oxidation of methionine residues affects the structure and stability of apolipoprotein A-I in reconstituted high density lipoprotein particles. Chem Phys Lipids 2001; 113:133-46. PMID: 11687233.
93. Sigalov A B, Stern U. Dihydrolipoic acid as an effective cofactor for peptide methionine sulfoxide reductase in enzymatic repair of oxidative damage to both lipid-free and lipid-bound apolipoprotein a-I. Antioxid Redox Signal 2002; 4:553-7. PMID: 12215223.
94. Yanachkov I B, Chang H, Yanachkova M I, Dix E J, Berny-Lang M A, Gremmel T, et al. New highly active antiplatelet agents with dual specificity for platelet P2Y1 and P2Y12 adenosine diphosphate receptors. Eur J Med Chem 2016; 107:204-18. PMID: 26588064.
95. Yanachkov I B, Dix E J, Yanachkova M I, Wright G E. P1,P2-diimidazolyl derivatives of pyrophosphate and bis-phosphonates—synthesis, properties, and use in preparation of dinucleoside tetraphosphates and analogs. Org Biomol Chem 2011; 9:730-8. PMID: 21082127.
96. Yanachkova M, Xu W C, Dvoskin S, Dix E J, Yanachkov I B, Focher F, et al. Prodrugs of herpes simplex thymidine kinase inhibitors. Antivir Chem Chemother 2015; 24:47-55. PMID: 26463822.
97. Walther D M, Mann M. Accurate quantification of more than 4000 mouse tissue proteins reveals minimal proteome changes during aging. Mol Cell Proteomics 2011; 10:M110 004523. PMID: 21048193.
98. Shelnutt S R, Gunnell M, Owens S M. Sexual dimorphism in phencyclidine in vitro metabolism and pharmacokinetics in rats. J Pharmacol Exp Ther 1999; 290:1292-8. PMID: 10454506.
99. Sigalov A, Alexandrovich O, Strizevskaya E. Large-scale isolation and purification of human apolipoproteins A-I and A-II. J Chromatogr 1991; 537:464-8. PMID: 1904881.
100. Sigalov A B. Comparison of apolipoprotein A-I values assayed in lyophilized and frozen pooled human sera by a non-immunochemical electrophoretic method and by immunoassay. Eur J Clin Chem Clin Biochem 1993; 31:579-83. PMID: 8260529.
101. Sigalov A B, Petrichenko I E, Kolpakova G V. The ratio of non-oxidized/oxidized forms of apolipoprotein A-I can affect cholesterol efflux from human skin fibroblasts mediated by high density lipoprotein. Eur J Clin Chem Clin Biochem 1997; 35:395-6. PMID: 9189746.
102. Zhang Y, Huo M, Zhou J, Xie S. PKSolver: An add-in program for pharmacokinetic and pharmacodynamic data analysis in Microsoft Excel. Comput Methods Programs Biomed 2010; 99:306-14. PMID: 20176408.
103. Scanu A, Hughes W L. Further characterization of the human serum D 1.063-1.21, alpha-lipoprotein. J Clin Invest 1962; 41:1681-9. PMID: 14497795.
104. Furman R H, Sanbar S S, Alaupovic P, Bradford R H, Howard R P. Studies of the Metabolism of Radioiodinated Human Serum Alpha Lipoprotein in Normal and Hyperlipidemic Subjects. J Lab Clin Med 1964; 63:193-204. PMID: 14125106.
105. Shen Z T, Sigalov A B. SARS Coronavirus Fusion Peptide-Derived Sequence Suppresses Collagen-Induced Arthritis in DBA/1J Mice. Sci Rep 2016; 6:28672. PMID: 27349522.
106. Yanachkov I, Zavizion B, Metelev V, Stevens L J, Tabatadze Y, Yanachkova M, et al. Self-neutralizing oligonucleotides with enhanced cellular uptake. Org Biomol Chem 2017; 15:1363-80. PMID: 28074950.
107. Huber L C, Distler J H, Moritz F, Hemmatazad H, Hauser T, Michel B A, et al. Trichostatin A prevents the accumulation of extracellular matrix in a mouse model of bleomycin-induced skin fibrosis. Arthritis Rheum 2007; 56:2755-64. PMID: 17665426.
108. Toyama T, Asano Y, Akamata K, Noda S, Taniguchi T, Takahashi T, et al. Tamibarotene Ameliorates Bleomycin-Induced Dermal Fibrosis by Modulating Phenotypes of Fibroblasts, Endothelial Cells, and Immune Cells. J Invest Dermatol 2016; 136:387-98. PMID: 26967475.
109. Yamamoto T, Takagawa S, Katayama I, Yamazaki K, Hamazaki Y, Shinkai H, et al. Animal model of sclerotic skin. I: Local injections of bleomycin induce sclerotic skin mimicking scleroderma. J Invest Dermatol 1999; 112:456-62. PMID: 10201529.
110. Yamamoto T, Takahashi Y, Takagawa S, Katayama I, Nishioka K. Animal model of sclerotic skin. II. Bleomycin induced scleroderma in genetically mast cell deficient WBB6F1-W/W(V) mice. J Rheumatol 1999; 26:2628-34. PMID: 10606374.
111. Avci P, Sadasivam M, Gupta A, De Melo W C, Huang Y Y, Yin R, et al. Animal models of skin disease for drug discovery. Expert Opin Drug Discov 2013; 8:331-55. PMID: 23293893.
112. Artlett C M. Animal models of systemic sclerosis: their utility and limitations. Open Access Rheumatol 2014; 6:65-81. PMID: 27790036.
113. Beyer C, Schett G, Distler O, Distler J H. Animal models of systemic sclerosis: prospects and limitations. Arthritis Rheum 2010; 62:2831-44. PMID: 20617524.
114. Ruzehaji N, Avouac J, Elhai M, Frechet M, Frantz C, Ruiz B, et al. Combined effect of genetic background and gender in a mouse model of bleomycin-induced skin fibrosis. Arthritis Res Ther 2015; 17:145. PMID: 26025306.
115. Bhattacharyya S, Wang W, Morales-Nebreda L, Feng G, Wu M, Zhou X, et al. Tenascin-C drives persistence of organ fibrosis. Nat Commun 2016; 7:11703. PMID: 27256716.
116. Hubner R H, Gitter W, El Mokhtari N E, Mathiak M, Both M, Bolte H, et al. Standardized quantification of pulmonary fibrosis in histological samples. Biotechniques 2008; 44:507-11, 14-7. PMID: 18476815.
117. Lakota K, Hanumanthu V S, Agrawal R, Carns M, Armanios M, Varga J. Short lymphocyte, but not granulocyte, telomere length in a subset of patients with systemic sclerosis. Ann Rheum Dis 2019. PMID: 30679155.
118. Yamashita T, Lakota K, Taniguchi T, Yoshizaki A, Sato S, Hong W, et al. An orally-active adiponectin receptor agonist mitigates cutaneous fibrosis, inflammation and microvascular pathology in a murine model of systemic sclerosis. Sci Rep 2018; 8:11843. PMID: 30087356.
119. Chang H I, Yeh M K. Clinical development of liposome-based drugs: formulation, characterization, and therapeutic efficacy. Int J Nanomedicine 2012; 7:49-60. PMID: 22275822.
120. Adler-Moore J P, Gangneux J P, Pappas P G. Comparison between liposomal formulations of amphotericin B. Med Mycol 2016; 54:223-31. PMID: 26768369.
121. Tricoci P, D'Andrea D M, Gurbel P A, Yao Z, Cuchel M, Winston B, et al. Infusion of Reconstituted High-Density Lipoprotein, CSL112, in Patients With Atherosclerosis: Safety and Pharmacokinetic Results From a Phase 2a Randomized Clinical Trial. J Am Heart Assoc 2015; 4:e002171. PMID: 26307570.
122. Shen Z T, Sigalov A B. Rationally designed ligand-independent peptide inhibitors of TREM-1 ameliorate collagen-induced arthritis. J Cell Mol Med 2017. PMID: 28382703.
123. Deshmukh S V, Durston J, Shomer N H. Validation of the use of nonnaive surgically catheterized rats for pharmacokinetics studies. J Am Assoc Lab Anim Sci 2008; 47:41-5. PMID: 19049252.
124. Wang X, Song L, Li N, Qiu Z, Zhou S, Li C, et al. Pharmacokinetics and biodistribution study of paclitaxel liposome in Sprague-Dawley rats and Beagle dogs by liquid chromatography-tandem mass spectrometry. Drug Res (Stuttg) 2013; 63:603-6. PMID: 23842945.
125. Kuwahara Y, Shima Y, Shirayama D, Kawai M, Hagihara K, Hirano T, et al. Quantification of hardness, elasticity and viscosity of the skin of patients with systemic sclerosis using a novel sensing device (Vesmeter): a proposal for a new outcome measurement procedure. Rheumatology (Oxford) 2008; 47:1018-24. PMID: 18440998.

### Additional Advantages of Using Peptides and Compositions as Described Herein.

As well-known in the art and described in Irby, et al. Mol Pharm 2017, 14:1325-1338, most anticancer chemotherapeutic agents as well as many other therapeutic agents (TA) are toxic and hydrophobic and cannot be administered by themselves as pure chemicals but have to be included in biocompatible formulations to enhance solubility, increase circulatory residence time of the therapeutic agents, minimize the undesirable side effects and alleviate drug resistance. Numerous formulation approaches have been developed, including solid lipid particles, emulsions, liposomes, etc., however, the delivery of the poorly water soluble (hydrophobic, or lipophilic) pharmaceuticals remains especially problematic as most of the body compartments, including the blood circulation and intracellular fluids, represent an aqueous environment. As a result, the direct injection of hydrophobic TAs often results in harmful side effects due to hypersensitivity, hemolysis, cardiac and neurological symptoms.

As described in Vlieghe, et al. Drug Discov Today 2010, 15:40-56, the main limitations generally attributed to therapeutic peptides are: a short half-life because of their rapid degradation by proteolytic enzymes of the digestive system and blood plasma; rapid removal from the circulation by the liver (hepatic clearance) and kidneys (renal clearance); poor ability to cross physiological barriers because of their general hydrophilicity; high conformational flexibility, resulting sometimes in a lack of selectivity involving interactions with different receptors/targets (poor specific biodistribution), causing activation of several targets and leading to side effects; eventual risk of immunogenic effects; and high synthetic and production costs (the production cost of a 5000 Da molecular mass peptide exceeds the production cost of a 500 Da molecular mass small molecule by more than 10-fold but clearly not 100-fold). Consequently, there is need for more effective and low toxic therapies for PC and other types of cancer as well as new formulations of hydrophobic drugs and therapeutic peptides to improve their targeted delivery, prolonged half-life, biocompatibility and therapeutic efficiency.

As described herein, it is surprisingly found that the peptides and compositions of the present invention capable of modulating the TREM-1 signaling pathway can be synthesized and used for targeted treatment of cancer and imaging. The advantageous trifunctional peptides and compositions are demonstrated by the present invention to solve numerous problems which otherwise are associated with high dosages of TAs and imaging probes required and the lack of control and reproducibility of formulations, especially in large-scale production.

As many other solid tumors, PC is characterized by a marked infiltration of macrophages into the stromal compartment (Shih 2006, Solinas 2009), a process, which is mediated by cancer-associated fibroblasts (CAFs) (FIG. 49) and plays a role in disease progression and its response to therapy. These tumor-associated macrophages (TAMs) secrete a variety of growth factors, cytokines, chemokines, and enzymes that regulate tumor growth, angiogenesis, invasion, and metastasis (Feurino 2006, Lewis and Pollard 2006, Shih 2006). High macrophage infiltration correlates with the promotion of tumor growth and metastasis development (Lin 2006, Lin 2001, Solinas 2009). In patients with PC, macrophage infiltration begins during the pre-invasive stage of the disease and increases progressively (Clark 2007). The number of TAMs is significantly higher in patients with metastases (Gardian 2012). Presence of TAMs in the PC stroma correlates with increased angiogenesis (Esposito 2004), a known predictor of poor prognosis (Kuwahara 2003). TAM recruitment, activation, growth and differentiation are regulated by macrophage colony-stimulating factor (M-CSF, also known as colony-stimulating factor 1, CSF-1) (Elgert 1998, Varney 2005). High pretreatment serum M-CSF is a strong independent predictor of poor survival in PC patients (Groblewska 2007). In PC mouse models, blockade of M-CSF or its receptor not only suppresses tumor angiogenesis and lymphangiogenesis (Kubota 2009) but also improves response to T-cell checkpoint immunotherapies that target programmed cell death protein 1 (PD-1) and cytotoxic T lymphocyte antigen-4 (CTLA-4) (Zhu 2014). Importantly, continuous M-CSF inhibition affects pathological angiogenesis but not healthy vascular and lymphatic systems outside tumors (Kubota 2009). In contrast to blockade of vascular endothelial growth factor (VEGF), interruption of M-CSF inhibition does not promote rapid vascular regrowth (Kubota 2009). Collectively, these findings further suggest that targeting TAMs is a promising strategy for treating cancer (Bowman and Joyce 2014, Jinushi and Komohara 2015, Komohara 2016).

Triggering receptor expressed on myeloid cells-1 (TREM-1) amplifies the inflammatory response (Colonna and Facchetti 2003) and is upregulated under inflammatory conditions including cancer (Ho et al. 2008, Yuan et al. 2014, Nguyen et al. 2015), brain and spinal cord injuries (Li et al 2019) and acute pancreatitis (D. Y. Wang 2004). For downstream signal transduction, TREM-1 is coupled to the immunoreceptor tyrosine-based activation motif (ITAM)-containing adaptor, DNAX activation protein of 12 kDa (DAP-12). Activation of TREM-1/DAP-12 receptor complex enhances release of multiple cytokines including monocyte chemoattractant protein-1 (MCP-1; also referred to in the art as CCL2), tumor necrosis factor-α (TNFα), interleukin-1α (IL-1α), IL-1β, IL-6 and macrophage colony-stimulating factor 1 (CSF-1; also referred to in the art as M-CSF) (Schenk et al. 2007, Dower et al. 2008, Sigalov 2014, Shen et al. 2017, Shen et al. 2017, Rojas et al. 2018, Tornai et al. 2019). Most of these cytokines are increased in cancer patients (Tjomsland et al. 2011, Rossi et al. 2015, Yako et al. 2016, Tsukamoto et al. 2018, Yoshimura 2018) and play a vital role in creating and sustaining inflammation in the tumor favorable microenvironment, thus affecting patient survival.

TREM-1 activation enhances release of multiple cytokines including monocyte chemoattractant protein-1 (MCP-1), tumor necrosis factor-α (TNFα), interleukin-1α (IL-1α), IL-1β, IL-6 and M-CSF (Lagler 2009, Schenk 2007, Sigalov 2014). Most of these cytokines are increased in patients with PC (Tjomsland 2011, Yako 2016) and play a vital role in creating and sustaining inflammation in the tumor favorable microenvironment, thus affecting patient survival. Inhibition of TREM-1 lowers levels of proinflammatory cytokines and is a promising approach in a variety of inflammation-associated disorders (Colonna and Facchetti 2003, Pelham and Agrawal 2014, Schenk 2007, Shen and Sigalov 2017, Sigalov 2014). Importantly, in contrast to cytokine blockers, blockade of TREM-1 can blunt excessive inflammation while preserving the capacity for microbial control (Weber 2014). In vitro silencing of TREM-1 suppresses cancer cell invasion (Ho 2008). In patients with non-small cell lung cancer (NSCLC), TREM-1 expression on TAMs is associated with cancer recurrence and poor survival: patients with low TREM-1 expression have a 4-year survival rate of over 60%, compared with less than 20% in patients with high TREM-1 expression (Ho 2008).

Inhibition of TREM-1 lowers levels of proinflammatory cytokines and chemokines including CSF-1 (Sigalov 2014, Shen and Sigalov 2017, Shen and Sigalov 2017, Rojas et al. 2018, Tornai et al. 2019) and as recently demonstrated in experimental cancer including NSCLC, pancreatic cancer and liver cancer, TREM-1 blockade inhibits tumor growth and improves survival (Wu et al. 2012, Sigalov 2014, Shen and Sigalov 2017, Wu et al. 2019). In vitro silencing of TREM-1 suppresses cancer cell invasion (Ho et al. 2008). In patients with NSCLC, TREM-1 expression on TAMs is associated with cancer recurrence and poor survival: patients with low TREM-1 expression have a 4-year survival rate of over 60%, compared with less than 20% in patients with high TREM-1 expression (Ho et al. 2008). Importantly, in contrast to cytokine blockers, blockade of TREM-1 can blunt excessive inflammation while preserving the capacity for microbial control (Weber et al. 2014). Septic mice lacking DAP-12, a signaling adapter of TREM-1, have improved survival (Turnbull et al. 2005). Humans lacking DAP12 do not have problems resolving infections (Lanier 2009). TREM-1 blockade is safe in healthy and septic subjects (Cuvier et al. 2018, Francois et al. 2018). Taken together, these finding make TREM-1 a promising therapeutic target in oncology.

The present invention provides the low toxic peptides and compositions for TREM-1-targeted treatment of cancer, e.g. PC, and other myeloid cell-related diseases and conditions and the methods for predicting the efficacy of these compositions. The invention further provides a method of using these peptides and compositions. These and other objects and advantages of the invention, as well as additional inventive features, will be apparent from the description of the invention provided herein.

FIG. 14A-C presents the exemplary data showing inhibition of tumor growth (FIG. 14A) and TREM-1 blockade-mediated suppression of intratumoral macrophage infiltration (FIG. 14B, FIG. 14C) in the human pancreatic cancer BxPC-3 xenograft mice treated with an equimolar mixture of the sulfoxidized methionine residue-containing TREM-1-related trifunctional peptides (TREM-1/TRIOPEP) GA 31 and GE 31 in free form or incorporated into synthetic lipopeptide particles (SLP) particles of discoidal (TREM-1/TRIOPEP-dSLP) and spherical (TREM-1/TRIOPEP-sSLP) morphology. (B) F4/80 staining. Results are expressed as the mean±SEM (n=4 mice per group). *, p<0.05; **, p<0.01, ****, p<0.0001 (versus vehicle). (FIG. 14C) Representative F4/80 images from BxPC-3-bearing mice treated using different free and sSLP-bound SCHOOL TREM-1 inhibitory GF9 sequences including TREM-1/TRIOPEP-sSLP. Scale bar=200 μm.

C. Sepsis; Severe Sepsis and Septic Shock.

Sepsis is another disorder with a high mortality rate. Currently, no approved sepsis drugs are available and over 30 drug candidates have failed late-stage clinical trials. Sepsis refers to a potentially life-threatening complication of an infection. Sepsis occurs when endogenous chemicals released into the bloodstream to fight the infection trigger inflammatory responses throughout the body. This inflammation can trigger a cascade of changes that can damage multiple organ systems, causing them to fail. If sepsis progresses to septic shock, blood pressure drops dramatically, which may lead to death.

Anyone can develop sepsis, but it's most common and most dangerous in older adults or those with weakened immune systems. Risk factors include but are not limited to: young or elderly; Have a compromised immune system; Are already very sick, often in a hospital's intensive care unit; Have wounds or injuries, such as burns; Have invasive devices, such as intravenous catheters or breathing tubes; etc.

Early treatment of sepsis, usually with antibiotics and large amounts of intravenous fluids, improves chances for survival. While any type of infection: including bacterial, viral or fungal, can lead to sepsis, the most likely varieties include: Pneumonia; Abdominal infection; Kidney infection; Bloodstream infection (bacteremia); etc.

The incidence of sepsis appears to be increasing in the United States. The causes of this increase may include: Aging population. Americans are living longer, which is swelling the ranks of the highest risk age group people older than 65; Drug-resistant bacteria. Many types of bacteria can resist the effects of antibiotics that once killed them. These antibiotic-resistant bacteria are often the root cause of the infections that trigger sepsis; Weakened immune systems. More Americans are living with weakened immune systems, caused by HIV, cancer treatments or transplant drugs; etc.

Sepsis ranges from less to more severe. As sepsis worsens, blood flow to vital organs, such as brain, heart and kidneys, becomes impaired. Sepsis can also cause blood clots to form in organs and in arms, legs, fingers and toes, leading to varying degrees of organ failure and tissue death (gangrene). Most people recover from mild sepsis, but the mortality rate for septic shock is nearly 50 percent. Also, an episode of severe sepsis may place you at higher risk of future infections.

Sepsis may present as a three-stage syndrome, starting with sepsis and progressing through severe sepsis to septic shock. The goal is to treat sepsis during its early stage, before it becomes more dangerous. the following symptoms, plus a probable or confirmed infection: Body temperature above 101 F (38.3 C) or below 96.8 F (36 C); Heart rate higher than 90 beats a minute; Respiratory rate higher than 20 breaths a minute, etc.

Severe sepsis refers to having at least one of the following signs and symptoms, which indicate an organ may be failing: Significantly decreased urine output; Abrupt change in mental status; Decrease in platelet count; Difficulty breathing; Abnormal heart pumping function; Abdominal pain; etc.

Septic shock refers to having at least one of the following signs and symptoms of severe sepsis, plus extremely low blood pressure that doesn't adequately respond to simple fluid replacement.

FIG. 15A-B presents the exemplary data showing improved survival of lipopolysaccharide (LPS)-challenged mice treated with an equimolar mixture of the sulfoxidized methionine residue-containing TREM-1-related trifunctional peptides (TREM-1/TRIOPEP) GA 31 and GE 31 in free form (FIG. 15A) or incorporated into synthetic lipopeptide particles (SLP) particles of discoidal (TREM-1/TRIOPEP-dSLP) and spherical (TREM-1/TRIOPEP-sSLP) morphology.
FIG. 15B. **, P=0.001 to 0.01 as compared with vehicle-treated animals.
FIG. 16 presents exemplary data showing average weights of healthy C57BL/6 mice treated with increasing concentrations of an equimolar mixture of the sulfoxidized methionine residue-containing TREM-1-related trifunctional peptides (TREM-1/TRIOPEP) GA 31 and GE 31 in free form.

D. Rheumatoid Arthritis (RA).

Rheumatoid arthritis (RA) refers to a chronic inflammatory disorder that can affect more than just your joints. In some people, the condition also can damage a wide variety of body systems, including the skin, eyes, lungs, heart and blood vessels.

RA affects as much as 1% of the worldwide population. There is no cure for RA yet and up to 80% or more of RA patients are disabled after 20 years of symptoms.

Unlike the wear-and-tear damage of osteoarthritis, rheumatoid arthritis affects the lining of your joints, causing a painful swelling that can eventually result in bone erosion and joint deformity.

The inflammation associated with rheumatoid arthritis is what can damage other parts of the body as well. While new types of medications have improved treatment options dramatically, severe rheumatoid arthritis can still cause physical disabilities.

Signs and symptoms of rheumatoid arthritis may include: Tender, warm, swollen joints; Joint stiffness that is usually worse in the mornings and after inactivity; Fatigue, fever and weight loss; etc.

Early rheumatoid arthritis tends to affect your smaller joints first, particularly the joints that attach your fingers to your hands and your toes to your feet.

As the disease progresses, symptoms often spread to the wrists, knees, ankles, elbows, hips and shoulders. In most cases, symptoms occur in the same joints on both sides of your body.

About 40 percent of the people who have rheumatoid arthritis also experience signs and symptoms that don't involve the joints. Rheumatoid arthritis can affect many nonjoint structures, including: Skin; Eyes; Lungs; Heart; Kidneys; Salivary glands; Nerve tissue; Bone marrow; Blood vessels; etc.

Rheumatoid arthritis signs and symptoms may vary in severity and may even come and go. Periods of increased disease activity, called flares, alternate with periods of relative remission—when the swelling and pain fade or disappear. Over time, rheumatoid arthritis can cause joints to deform and shift out of place.

Rheumatoid arthritis increases your risk of developing: Osteoporosis. Rheumatoid arthritis itself, along with some medications used for treating rheumatoid arthritis, can increase your risk of osteoporosis—a condition that weakens your bones and makes them more prone to fracture. Rheumatoid nodules. These firm bumps of tissue most commonly form around pressure points, such as the elbows. However, these nodules can form anywhere in the body, including the lungs. Dry eyes and mouth. People who have rheumatoid arthritis are much more likely to experience Sjogren's syndrome, a disorder that decreases the amount of moisture in your eyes and mouth. Infections. The disease itself and many of the medications used to combat rheumatoid arthritis can impair the immune system, leading to increased infections. Abnormal body composition. The proportion of fat compared to lean mass is often higher in people who have rheumatoid arthritis, even in people who have a normal body mass index (BMI). Carpal tunnel syndrome. If rheumatoid arthritis affects your wrists, the inflammation can compress the nerve that serves most of your hand and fingers. Heart problems. Rheumatoid arthritis can increase your risk of hardened and blocked arteries, as well as inflammation of the sac that encloses your heart. Lung disease. People with rheumatoid arthritis have an increased risk of inflammation and scarring of the lung tissues, which can lead to progressive shortness of breath. Lymphoma. Rheumatoid arthritis increases the risk of lymphoma, a group of blood cancers that develop in the lymph system.

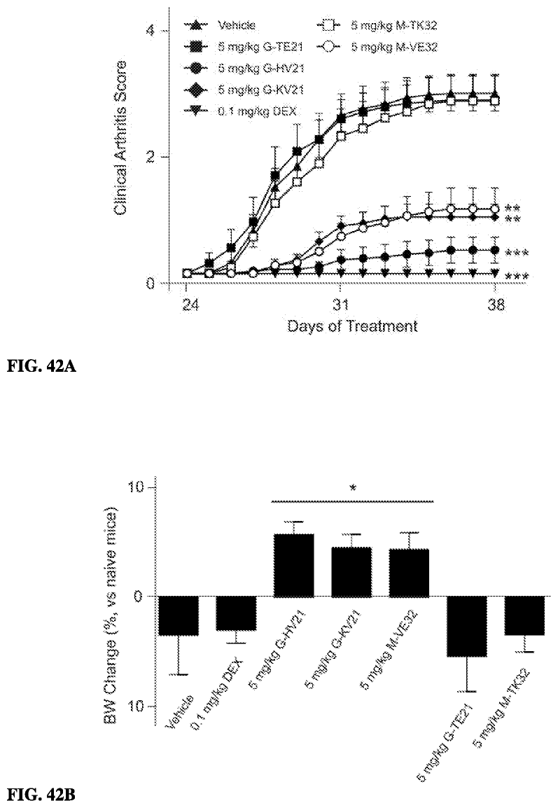

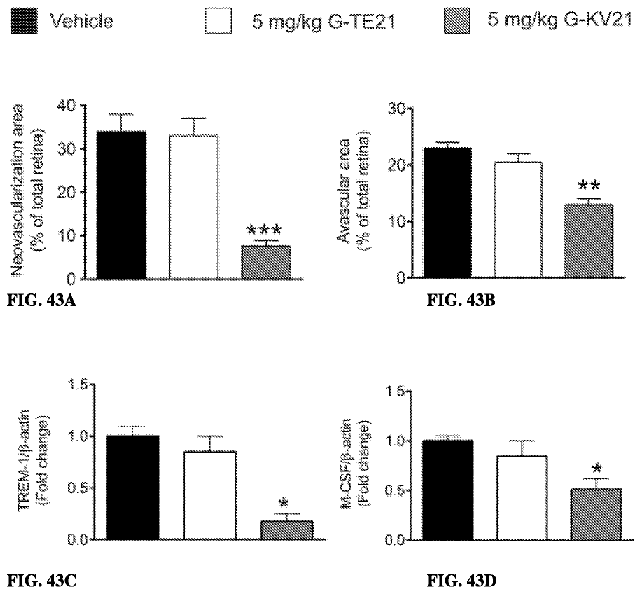

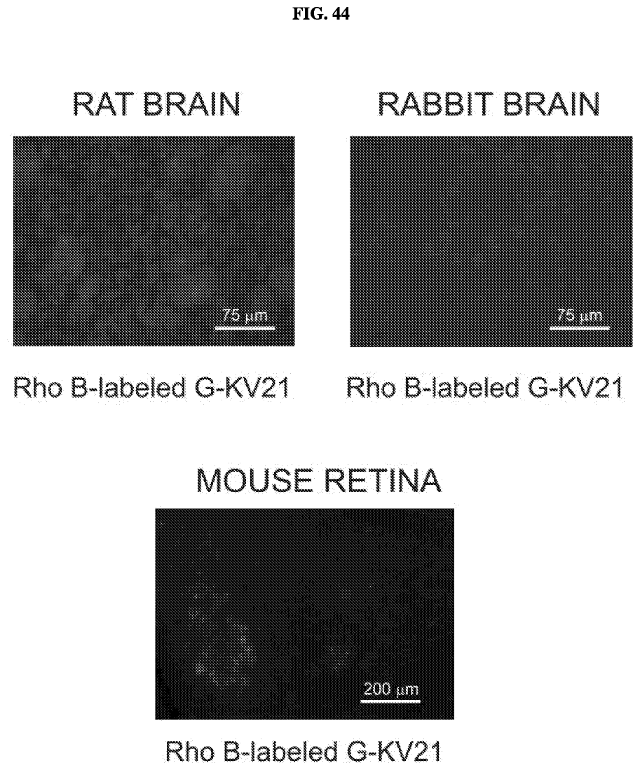

FIG. 17A-B presents the exemplary data showing average clinical arthritis score (FIG. 17A) and mean body weight (BW) changes (FIG. 17B) calculated as a percentage of the difference between beginning (day 24) and final (day 38) BWs of the collagen-induced arthritis (CIA) mice treated with an equimolar mixture of the sulfoxidized methionine residue-containing TREM-1-related trifunctional peptides (TREM-1/TRIOPEP) GA 31 and GE 31 incorporated into synthetic lipopeptide particles (SLP) particles of discoidal (TREM-1/TRIOPEP-dSLP) and spherical (TREM-1/TRIOPEP-sSLP) morphology. DEX, dexamethasone. *, p<0.05, **, p<0.01; ***, p<0.001 as compared with vehicle-treated or naive animals.
FIG. 42A-B presents exemplary data showing average clinical arthritis score (Collagen-induced arthritis: Score 42A) and Collagen-induced arthritis: Body weight change mean BW changes (42B) calculated as a percentage of the difference between beginning (day 24) and final (day 38) BWs of the CIA mice treated with PBS (vehicle), DEX, TREM-1-related control peptide G-TE21, TCR-related control peptide M-TK32, TCR-related trifunctional peptide M-VE32 or with TREM-1-related trifunctional peptides G-HV21 and G-KV21. In contrast to the relevant control peptides, G-HV21, G-KV21 and M-VE32 all ameliorate the disease (A) and are well-tolerated by arthritic mice (B). *, p<0.05, **, p<0.01; ***, p<0.001 as compared with vehicle-treated animals. Abbreviations: TREM-1, triggering receptor expressed on myeloid cells-1; CIA, collagen-induced arthritis; PBS, phosphate-buffer saline; DEX, dexamethasone; TCR, T cell receptor; BW, body weight.

E. Retinopathy.

Pathological retinal neovascularization (RNV) causes angiogenesis-related vision impairment in retinopathy of prematurity (ROP), diabetic retinopathy (DR), and retinal vein occlusion (RVO), which are the most common causes of vision loss and blindness in each age group. Conventional therapeutic options include laser ablation and the anti-vascular endothelial growth factor (VEGF) therapy, which both have their limitations and complications. Laser therapy is often accompanied by corneal edema, anterior chamber reaction, intraocular hemorrhage, cataract formation, and intraocular pressure changes, while the VEGF-targeted therapy can be complicated by damage of healthy vessels, potential side effects on neurons, rapid vascular regrowth upon interrupting the VEGF blockade, and limited effectiveness in some patients.

F. Cirrhosis of the Liver and Alcoholic Liver Disease.

The human liver is located in the upper right side of the abdomen below the ribs. It has many essential body functions. These include: producing bile, which helps your body absorb dietary fats, cholesterol, and vitamins A, D, E, and K; storing sugar and vitamins for later use by the body; removing toxins such as alcohol and bacteria from your system: creating blood clotting proteins; etc.

Several of the most common causes of cirrhosis of the liver in the United States are long-term viral hepatitis C infection and chronic alcohol abuse. Obesity is also a cause of cirrhosis, although it is not as prevalent as alcoholism or hepatitis C. Obesity can be a risk factor by itself, or in combination with alcoholism and hepatitis C.

According to The National Institute of Diabetes and Digestive and Kidney Diseases (NIDDK) and other components of the National Institutes of Health (NIH), cirrhosis can develop in women who drink more than two alcoholic drinks per day (including beer and wine) for many years. For men, drinking more than three drinks a day for years can put them at risk for cirrhosis.

However, the amount is different for every person, and this doesn't mean that everyone who has ever drunk more than a few drinks will develop cirrhosis. Cirrhosis caused by alcohol is usually the result of regularly drinking more than these amounts over the course of 10 or 12 years. Cirrhosis causes the liver to shrink and harden. This makes it difficult for nutrient-rich blood to flow into the liver from the portal vein. The portal vein carries blood from the digestive organs to the liver. The pressure in the portal vein rises when blood can't pass into the liver. The end result is a serious condition called portal hypertension, in which the vein develops high blood pressure. The unfortunate consequence of portal hypertension is that this high-pressure system causes a backup, which leads to esophageal varices (like varicose veins), which can then burst and bleed. Cirrhosis of the liver refers to severe scarring of the liver and poor liver function seen at the terminal stages of chronic liver disease. The scarring is most often caused by long-term exposure to toxins such as alcohol or viral infections.

Alcoholic liver cirrhosis is directly related to alcohol intake and is the final phase of alcoholic liver disease. Symptoms including but not limited to: anemia (low blood levels due to too little iron); high blood ammonia level); high blood sugar levels; leukocytosis (large amount of white blood cells); unhealthy liver tissue when a sample is removed from a biopsy and studied in a laboratory; liver enzyme blood tests that show the level of aspartate aminotransferase (AST) is two times that of alanine aminotransferase (ALT); low blood magnesium levels; low blood potassium levels; low blood sodium levels; portal hypertension; etc.

Alcoholic liver cirrhosis can cause serious complications. This is known as decompensated cirrhosis. Examples of these complications include: ascites, or a buildup of fluid in the stomach; encephalopathy, or mental confusion; internal bleeding, known as bleeding varices; jaundice, which makes the skin and eyes have a yellow tint; etc.

Those with this the more severe form of cirrhosis often require a liver transplant to survive; etc. According to the Cleveland Clinic, patients with decompensated alcoholic liver cirrhosis who receive a liver transplant have a five-year survival rate of 70 percent.

Alcoholic liver disease (ALD) occurs after years of heavy drinking. The chances of getting liver disease go up the longer you have been drinking and more alcohol you consume. Typically, a person has drank heavily for at least eight years. The National Institute on Alcohol Abuse and Alcoholism defines heavy drinking as drinking five or more drinks in one day on at least five of the past 30 days.

Symptoms of alcoholic liver cirrhosis typically develop when a person is between the ages of 30 and 40. A human body will be able to compensate for it's liver's limited function in the early stages of the disease. As the disease progresses, symptoms will become more noticeable. The disease is common in people between 40 and 50 years of age. Men are more likely to have this problem. However, Women are also more at-risk for alcoholic liver disease. Women don't have as many enzymes in their stomachs to break down alcohol particles. Because of this, more alcohol is able to reach the liver and make scar tissue.

Alcoholic liver disease can also have some genetic factors. For example, some people are born with a deficiency in enzymes that help to eliminate alcohol. Obesity, a high-fat diet, and having hepatitis C can also increase a person's likelihood they will have alcoholic liver disease. women may develop the disease after less exposure to alcohol than men. Some people may have an inherited risk for the disease. The disease is part of a progression. It may start with fatty liver disease, then progress to alcoholic hepatitis, and then to alcoholic cirrhosis. However, it's possible a person can develop alcoholic liver cirrhosis without ever having alcoholic hepatitis.

When a person drinks alcohol heavily over the course of decades, the body starts to replace the liver's healthy tissue with scar tissue. Doctors call this condition alcoholic liver cirrhosis.

Alcoholic liver disease affects millions of people globally and often leads to fibrosis and cirrhosis. Liver cirrhosis is the 12th leading cause of death in the United States and costs society more than $15 billions annually. Despite this profound economic and health impact, there are currently no approved drugs to treat ALD. Current treatments including corticosteroids, immunosuppressants, and antioxidants have multiple shortcomings including a high level of serious side effects and insufficient efficacy. slow the disease's progress and reduce your symptoms.

In some emboidments, either or both of the TREM-1 rHDLS and TREM-1 trifunctional peptides may be used in combination with treatments including but not limited to: Medications: including but not limited to corticosteroids, calcium channel blockers, insulin, antioxidant supplements, and S-adenosyl-L-methionine (SAMe); Nutritional Counseling: Alcohol abuse can lead to malnutrition; Extra protein: Patients often require extra protein in certain forms to help reduce the likelihood for developing brain disease (encephalopathy); Liver Transplant; etc. investigated the role of TREM-1 in ALD and the potential therapeutic effect of the TREM-1 inhibitory GF9-HDL and GA/E31-HDL formulations in the Lieber-DeCarli ALD mouse model.

1. Trem-1 Blockade Ameliorates Expression of Early Fibrosis Marker Genes Induced by Chronic Alcohol Consumption.

The clinical progression of ALD is associated with liver fibrosis.²⁷ Our mouse model of ALD mimics the early phase of the human disease, yet mRNA levels of early fibrosis markers Pro-Colla and a-SMA were significantly increased in alcohol-fed mice compared to PF controls in the whole-liver samples (FIG. 20A-B). Induction of these makers was remarkably attenuated in the vehicle-treated group and further decreased by the TREM-1 inhibitory formulations used (FIG. 20A-B).

FIG. 20A-B presents exemplary data showing TREM-1/TRIOPEP-sSLP suppresses the expression of fibrinogenesis marker molecules, FIG. 20A Pro-Collagen 1α and FIG. 20B α-Smooth Muscle Actin, at the RNA level, as measured in whole-liver lysates of mice with (alcohol-fed) and without (pair-fed) alcoholic liver disease (ALD).
* indicates significance level compared to the non-treated pair-fed (PF) group; #indicates significance level compared to the non-treated alcohol-fed group. o indicates significance level compared to the vehicle-treated alcohol-fed group. The significant levels are as follows: *, 0.05≥P≥0.01; **, 0.01≥P≥0.001; ***, 0.001≥P≥0.0001; ****, P≤0.0001.

2. Trem-1 Inhibitory Formulations and HDL Ameliorate Chronic Alcohol-Induced Liver Injury and Steatosis.

We evaluated the impact of the TREM-1 inhibitors on hepatocyte damage and steatosis in liver. Serum ALT levels obtained during week 5 of the alcohol feeding showed significant increases in alcohol-fed mice compared to PF controls. This ALT increase was attenuated in both TREM-1 inhibitor-treated groups, indicating attenuation of liver injury (FIG. 21A). Interestingly, vehicle treatment (HDL) also showed a similar protective effect (FIG. 21A).

Consistent with steatosis, we found a significant increase in Oil Red O staining in livers of alcohol-fed mice compared to PF controls (FIG. 21C). Oil Red O (FIG. 21B-D) and H&£ (FIG. 21D) staining revealed attenuation of steatosis in the alcohol-fed TREM-1 inhibitor-treated mice compared to both untreated and vehicle (HDL)-treated alcohol-fed groups (FIG. 21B-D).

FIG. 21A-D presents exemplary data showing that TREM-1/TRIOPEP-sSLP suppresses the production of alanine aminotransferase (ALT) in mice with alcoholic liver disease (ALD), as measured in serum of mice with (alcohol-fed) and without (pair-fed) ALD, in addition to improving indicators of liver disease and inflammation. * indicates significance level compared to the alcohol-fed group treated with vehicle-synthetic lipopeptide particles of spherical morphology that contained an equimolar mixture of PE22 and PA22 (sSLP) but no TREM-1 inhibitory peptide GF9. #indicates significance level compared to the non-treated alcohol-fed group. Liver damage after 5 weeks of alcohol feeding and effect of TREM-1 pathway inhibition in a mouse model of ALD. sSLP, 5 mg/kg treatment of TREM-1 peptide vs. TREM-1/TRIOPEP-sSLP. Cheek blood and livers were harvested at death. (FIG. 21A) Serum ALT levels were measured using a kinetic method. Exemplary data showing TREM-1/TRIOPEP-sSLP suppresses alanine aminotransferase in serum of alcohol fed mice over TREM-1 peptide alone. (FIG. 21B-D) Liver sections were stained with (B,C) Oil Red O and (FIG. 21D) H&E staining, and the lipid content was analyzed by ImageJ (FIG. 21B). * indicates significance level compared to the nontreated PF group; * indicates significance level compared to the nontreated alcohol-fed group; ⁰ indicates significance level compared to the vehicle-treated alcohol-fed group. The numbers of the symbols sign the significant levels as the following: **^{o}P≤0.05; ^{##/oo}P≤0.01; *″^{/##}P≤0.001; ****P≤0.0001. ***, 0.001≥P≥0.0001; ##, 0.01≥P≥0.001.

3. Blockade of Trem-1 Signaling Reduces the Expression of Inflammation-Associated Genes in ALD in Mice.

Previous reports showed that TREM-1 activation leads to the expression and release of proinflammatory cytokines and chemokines through nuclear factor kB activation, which also regulates the expression of TREM-1, providing a positive feedback loop on the expression of the receptor.⁴ Proinflammatory cytokine expression is increased in ALD\hat{ }^{1-3,23,24}, therefore, we hypothesized that TREM-1 signaling contributes to the amplification of proinflammatory pathways in ALD.

To evaluate this hypothesis, first we tested whole-liver mRNAs of EtOH-fed and PF mice with or without treatment with two different TREM-1 inhibitory formulations and a vehicle control in a 5-week alcohol administration model of ALD in mice.⁽²⁵⁾ We found that mRNA levels of TREM-1 and MCP-1 were significantly increased in livers of alcohol-fed mice compared to PF controls (FIG. 1A,B).

In contrast, in mice treated with the TREM-1 inhibitors, both GF9-HDL and GA/E31-HDL inhibited alcohol-related changes in TREM-1; in addition, MCP-1 mRNA levels corresponded to those of the PF controls (FIG. 1A,B). Although induction of TNF-a and IL-lls in alcohol-fed mice did not reach statistical significance compared to PF controls, TREM-1 blockade by GF9-HDL resulted in a significant inhibition of TNF-a mRNA in the alcohol-fed mice compared to vehicle treatment (FIG. 1C), while IL-lfi mRNA expression was also significantly attenuated by both the GF9-HDL and GA/E31-HDL formulations in the alcohol-fed as well as in the PF groups (FIG. 1D). MIP-1a mRNA levels were increased in alcohol-fed mice, but TREM-1 blockade with GF9-HDL or GA/E31-HDL significantly attenuated this increase compared to the vehicle control (FIG. 1E). Regulated on activation, normal T cell expressed, and secreted (RANTES) mRNA levels did not change regardless of alcohol feeding or TREM-1 treatment (FIG. 1F).

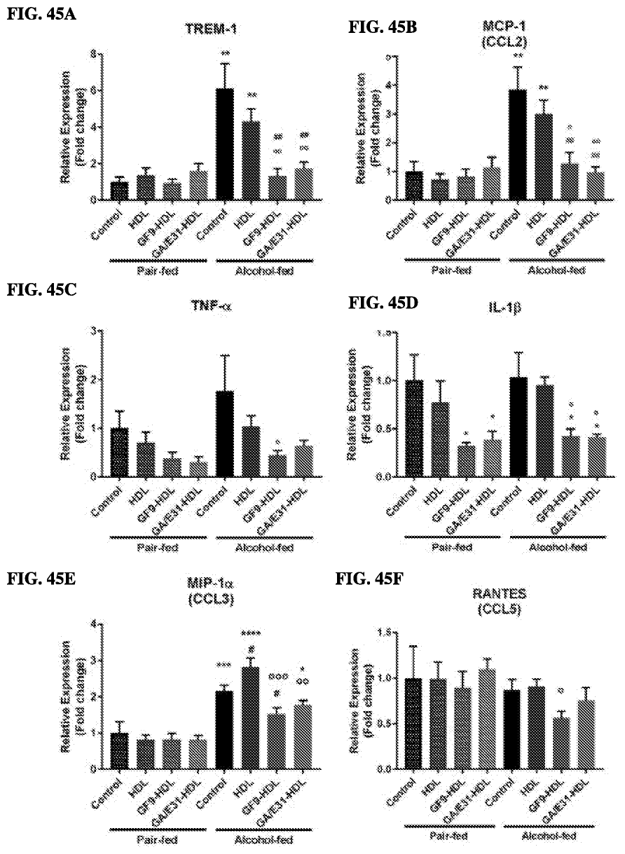

FIG. 45A-E TREM-1 pathway inhibition. TREM-1 pathway inhibition suppresses the expression of (FIG. 45A) TREM-1 and inflammatory cytokines (FIG. 45B) MCP-1, (FIG. 45C) TNF-α, (FIG. 45D) IL-1, and (FIG. 45E) MIP-1α but not (F) RANTES at the mRNA level as measured in whole-liver lysates by real-time quantitative PCR. * indicates significance level compared to nontreated PF group; #indicates significance level compared to nontreated alcohol-fed group; o indicates significance level compared to vehicle-treated alcohol-fed group. Significance levels are as follows: */#/o P≤0.05; **/##/oo P≤0.01; ***/ooo P≤0.001; ****P≤0.0001. Abbreviation: CCL, chemokine (C—C motif) ligand.

Next, we used specific ELISA kits to assess the protein levels of cytokines in the serum and in whole-liver lysates (FIG. 2). We found a significant increase in MCP-1 level in the serum and liver and TNF-a in the liver of alcohol-fed mice compared to PF controls (FIG. 2A-D). All these alcohol-induced increases were prevented both in the serum and liver by administration of either TREM-1 inhibitor. Interestingly, we found attenuation of alcohol-induced liver MCP-1 and TNF-a induction even in the vehicle-treated (HDL only) groups (FIG. 2A-C). The increase in total IL-lfs levels after alcohol feeding and its attenuation by TREM-1 inhibition did not reach statistical significance (FIG. 2D).

Because TREM-1 is a membrane-associated molecule that triggers SYK activation as one of its proximal signaling molecules and we previously found increased SYK phosphorylation in liver in ALD/²⁴″ we EB tested the levels of total and activated phospho-SYK (p-SYKY^{525/526}) in the livers. We found significantly increased total and p-SYK^{Y525/526} levels after alcohol feeding (FIG. 2E-G). Treatment with GA/E31-HDL significantly decreased the p-SYK^{YS2S/526} levels in alcohol-fed mice compared to the untreated and vehicle-treated alcohol-fed groups, while GF9-HDL decreased p-SYK^{YS25/526} levels compared to the vehicle-treated group. (FIG. 2E,F).

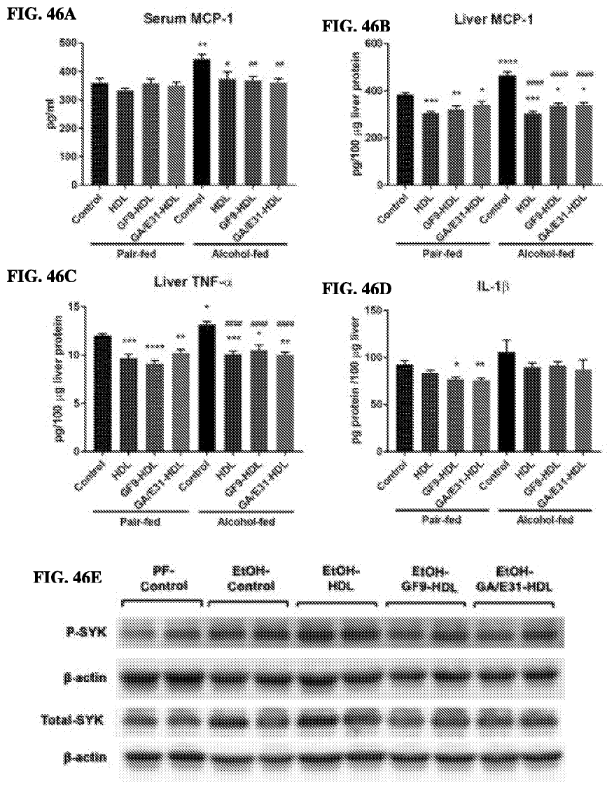

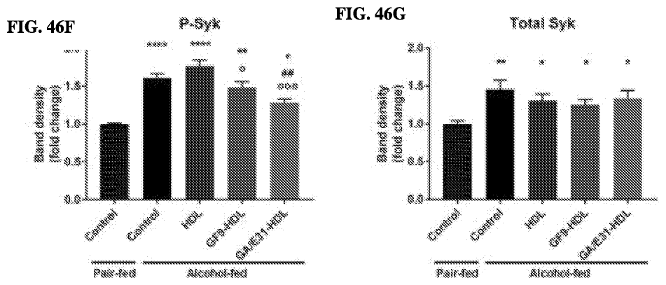

FIG. 46AE-G TREM-1 blockade and inflammatory cytokine levels. TREM-1 blockade reduces inflammatory cytokine levels in (FIG. 46A) serum and (FIG. 46B-D) whole-liver lysates as measured with specific ELISA kits. (FIG. 46E-G) Total liver protein was analyzed for total SYK and activated p-SYK Y525/526 expression by western blotting using j-actin as a loading control. Statistical analysis was performed by evaluating two blots (n=4/group). * indicates significance level compared to the nontreated PF group; #indicates significance level compared to the nontreated alcohol-fed group; o indicates significance level compared to the vehicle-treated alcohol-fed group. Significance levels are as follows: */#/o P≤0.05; **/##P≤0.01; ***P≤0.001; ****/####P≤0.0001.

5. Blockade of Trem-1 Activation Reduces Expression of Macrophage and Neutrophil Markers in Liver

In agreement with previous studies indicating that chronic alcohol use causes hepatic macrophage infiltration and activation/^{1,3,26}\hat{ } we found increased expression of the Kupffer cell/macrophage markers F4/80 and CD68 at the mRNA level. Treatment with the TREM-1 inhibitors significantly attenuated alcohol-induced expression of both F4/80 and CD68 in the liver, indicating anti-significant decrease in F4/80 expression on paraffin-embedded liver sections by IHC in alcohol-fed mice treated with either GF9-HDL or GA/E31-HDL compared to the EtOH-fed vehicle-treated group (FIG. 3C,D).

Neutrophil infiltration of the liver is a characteristic of alcoholic hepatitis; therefore, we investigated markers associated with this cell population. Expression of the neutrophil markers Ly6G and MPO were significantly increased in livers of alcohol-fed mice compared to PF controls. This was fully prevented by TREM-1 blockade (FIG. 3E,F). Interestingly, the HDL vehicle alone also resulted in a decreasing trend of Ly6G and MPO expression in alcohol-fed mice; however, the GF9-HDL and GA/E31-HDL TREM-1 inhibitors significantly attenuated Ly6G and MPO levels even when compared to the vehicle-treated alcohol-fed mice (FIG. 3E,F). MPO staining on IHC confirmed that both TREM-1 inhibitors significantly reduced MPO-positive cell numbers compared to the untreated alcohol-fed control group (FIG. 3G,H).

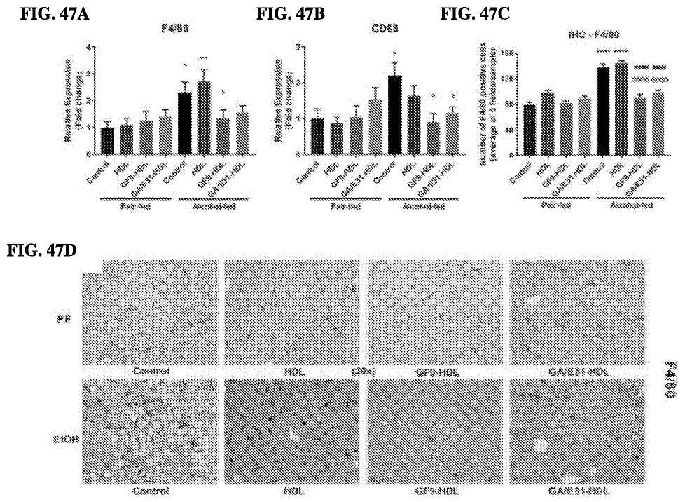

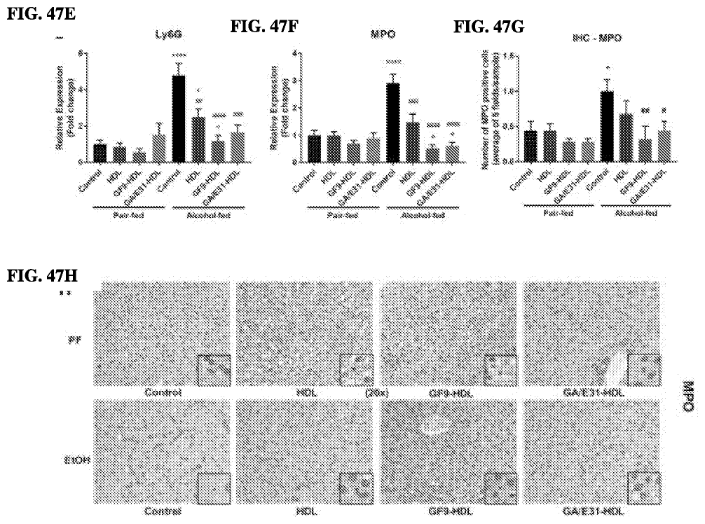

FIG. 47A-H. Effects of TREM-1 inhibition. (FIG. 47A, FIG. 47B) TREM-1 inhibition suppresses the mRNA expression of macrophage cell markers in the liver as measured by real-time quantitative PCR. (FIG. 47C, FIG. 47D) Both TREM-1 inhibitors attenuated F4/80 as shown by IHC. (FIG. 47E, FIG. 47F) TREM-1 inhibition suppresses the mRNA expression of neutrophil cell markers in the liver as measured by real-time quantitative PCR. (FIG. 47G, H) Both TREM-1 inhibitors attenuated MPO-positive cell infiltration as shown by IHC. * indicates significance level compared to the nontreated PF group; #indicates significance level compared to the nontreated alcohol-fed group; o indicates significance level compared to the vehicle-treated alcohol-fed group. Significance levels are as follows: */#/o P≤0.05; **/##P≤0.01; ###P≤0.001; ****/####P≤0.0001.

6. Trem-1 Inhibitory Formulations and HDL Ameliorate Chronic Alcohol-Induced Liver Injury and Steatosis

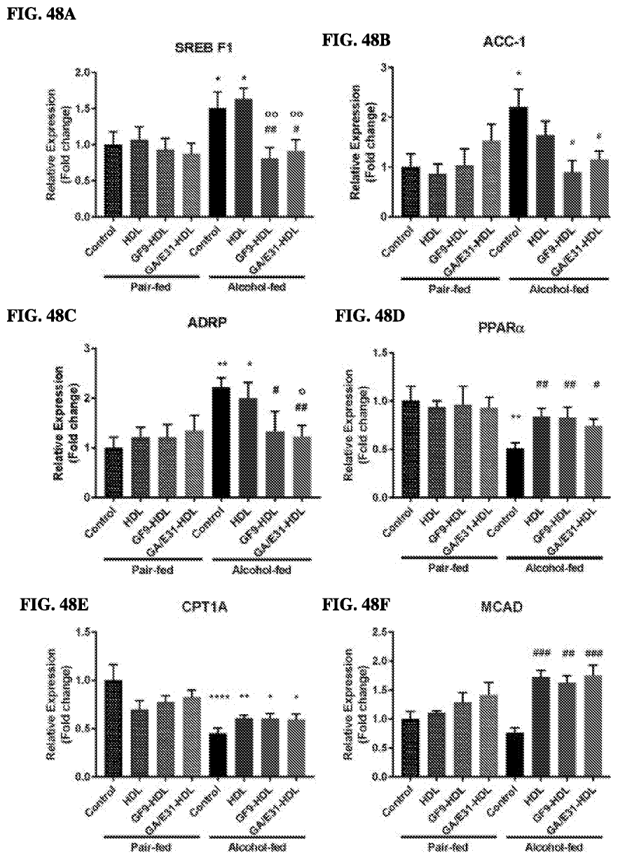

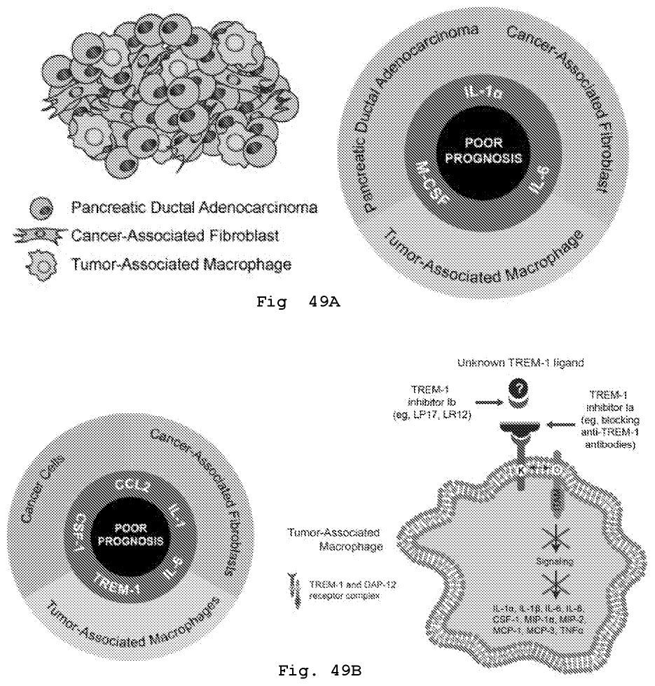

To further assess the effects of the TREM-1 inhibitors on mechanisms of lipid metabolism, we tested genes involved in lipid synthesis (sterol regulatory element binding transcription factor 1 [SREBF1] and acetyl-coenzyme A carboxylase 1 [ACC1]) along with the lipid accumulation marker perilipin-2 (ADRP) (FIG. 48A-C). Both TREM-1 inhibitors but not vehicle treatment prevented alcohol-induced up-regulation of SREBF1, ACC1, and ADRP at the mRNA level (FIG. 48A-C). To assess lipid oxidation, we tested peroxisome proliferator-activated receptor a (PPARa), carnitine palmitoyl transferase 1A (CPT1A), and medium-chain acyl-coenzyme A dehydrogenase (MCAD) mRNA levels in whole-liver samples (FIG. 48D-F). Alcohol feeding significantly reduced mRNA expression of PPARa and CPT1A, while MCAD had a decreasing trend. Both TREM-1 inhibitors as well as the vehicle treatment significantly increased PPARa and MCAD levels compared to the untreated alcohol-fed controls (FIG. 48D-F).

FIG. 48A-F Measurement of mRNA expression. mRNA expression of genes involved in (FIG. 48A, FIG. 48B) lipid synthesis (SERBF1, ACC1), (FIG. 48C) the lipid accumulation marker (ADRP), and (FIG. 48D-F) lipid oxidation (PPARα, CPT1α, MCAD) were measured in whole liver.
* indicates significance level compared to the nontreated PF group; #indicates significance level compared to the nontreated alcohol-fed group; o indicates significance level compared to the vehicle-treated alcohol-fed group. Significance levels are as follows: */#/o P≤0.05; **/##/oo P≤0.01; ###P≤0.001; ****P≤0.0001.

7. GF9HDL and GA/E31HDL is Mainly Mediated by SRA

We studied the uptake of GF9-HDL and GA/E31-HDL in vitro in order to evaluate potential mechanisms of targeted delivery of GF9 (GA/E31). Kupffer cells and recruited hepatic macrophages express high levels of SRs, including SR-A, that are involved in phagocytosis and removal of oxidatively damaged lipoproteins and cells from the blood circulation.^{28,29} We previously demonstrated intracellu-SR lar macrophage delivery of GF9, GA31, and GE31 by macrophage-targeted GF9-HDL and GA/E31-HDL, respectively, and hypothesized that the observed macrophage endocytosis of these complexes is SR mediated.^{16,17} See, FIGS. 9A1 and 9A2. To further investigate the molecular mechanisms involved in this process, we used J774 macrophages as a model for Kupffer cells and incubated them with rho B-labeled GF9-HDL or GA/E31-HDL in the presence or absence of cytochalasin D, fucoidan, or BLT-1, which are known to inhibit all SRs,⁽³⁰⁾ SR-A,⁽³¹⁾ or SR-BI,⁽³²⁾ respectively.

In the presence of cytochalasin D, which inhibits both SR-A and SR-BI, the macrophage uptake of both TREM-1 inhibitor complexes was significantly inhibited, suggesting that this uptake is SR mediated. Fucoidan, an SR-A inhibitor, substantially suppressed endocytosis of TREM-1 inhibitor complexes at 22 hours but not at 4 hours, indicating time-dependent mechanisms of SR-A-mediated endocytosis (FIG. 9B). In contrast, BLT-1, which inhibits SR-BI, similarly inhibited the uptake of the complexes at both time points but to a lesser extent compared with that of fucoidan (FIG. 9C), presumably because of lower expression of SR-BI on J774 macrophages^{(33,1)} These findings suggest that SR-A is the main contributor in SR-mediated endocytosis of both GF9-HDL and GA/E31-HDL.

Interestingly, quantitatively determined macrophage uptake levels in the presence or absence of fucoidan or BLT-1 were similar for GF9-HDL and GA/E31-HDL (FIG. 7B). This suggests that the combination of GF9 and apo AT peptide sequences in GA31 and GE31 sequences does not change the level and mechanisms of macrophage endocytosis of GA/E31-HDL compared with those of GF9-HDL.

8. Summary of TREM-1 in ALD.

Using a mouse model, significant up-regulation of TREM-1 was measured in livers of mice following chronic alcohol feeding. Treatment with novel ligand-independent TREM-1 inhibitors reduced the expression of the TREM-1 molecule itself, attenuated or fully prevented alcohol-induced increases in proinflammatory cytokines at the mRNA level, and inhibited SYK activation. TREM-1 blockade provided by trifunctional peptides described herein, results in reduced macrophage and neutrophil infiltration and activation indicated by reduced F4/80, CD68, Ly6G, and MPO expression in the liver. These findings complement data demonstrating that TREM-1 blockade using GF9-HDL and GA/E31-HDL suppresses macrophage infiltration of the tumor in cancer mice. (Reference 17) The TREM-1 inhibitors attenuated alcohol-induced liver steatosis. HDL and the TREM-1 inhibitors also attenuated liver injury and markers of early fibrosis in alcohol-fed mice. Interestingly, the HDL vehicle control showed similar efficiency as the inhibitory formulations at the protein level of the proinflammatory cytokines. Theefore it was also discovered that rHDL itself has some protective effects on ALD at the level of ALT and lipid oxidation.

While the ligand of TREM-1 is still unknown, it has been shown that TREM-1 activation amplifies inflammation and synergizes with TLR signaling pathways. (34) It was also observed that bacterial infection and challenge with LPS or lipoteichoic acid increase TREM-1 expression, (7) indicating a positive feedback loop among PAMP exposure, TREM-1 expression, and inflammatory cytokine induction. Different DAMPs, such as 3-hydroxy-3-methyl-glutaryl Bl and heat shock protein 70, have been suggested to stimulate TREM-1, (35)′ while other studies found cell (granulocyte and platelet)-surface-associated activators as well. (35, 36) Both PAMPs and DAMPs are present in ALD, providing potential mechanisms for TREM-1 up-regulation in this disease. Alcohol induces changes in the gut microbiome and disrupts the gut barrier function, resulting in increased levels of endotoxin and microbial PAMPs in circulation. (1, 37) Alcohol also causes hepatocyte damage that leads to the release of DAMPs, (23) and these processes contribute to TREM-1 activation.

TREM-1 signaling leads to phosphorylation and activation of SYK, which has been indicated as a major regulator in inflammatory processes in ALD.⁽³⁸⁾ TREM-1 also amplifies TLR4 signaling that involves activation of SYK, which has been indicated as a downstream SYK activation and phosphorylation.⁽³⁸⁾ Indeed, we found increased total and phosphorylated SYK levels in the livers of alcohol-fed mice that was attenuated by TREM-1 inhibitor administration. A previous study showed that inhibition of SYK activation attenuates alcohol-induced liver inflammation, cell death, and steatosis, suggesting that the SYK pathway could be a feasible therapeutic target in ALD.⁽²⁴⁾ SYK is expressed in a wild spectrum of cells, while TREM-1 inhibition may specifically modulate macrophages, neutrophils, and stellate cells that each play a role in ALD. Another advantage of TREM-1 inhibition is that it likely attenuates signaling from a broader spectrum of TLRs, in addition to TLR4.

TREM-1 activation alone has been shown to increase the production of proinflammatory chemo-kines and cytokines.⁽³⁹⁾ Furthermore, simultaneous stimulation of TREM-1 and TLRs by an agonistic anti-TREM-1 antibody and different TLR ligands synergized in the induction of these proinflammatory molecules. TREM-1 and TLR4 costimulated monocytes showed increased production of MCP-1, IL-1β, and IL-8. In contrast, the level of the anti-inflammatory cytokine IL-10 decreased when anti-TREM-1 antibody and the TLR3 ligand poly(LC) or the TLR4 ligand LPS simultaneously attached to their receptors.⁽⁴⁰⁾ Because self-perpetuating proinflammatory pathways are present in alcoholic hepatitis, interruption of these pathways using TREM-1 inhibition seems attractive.

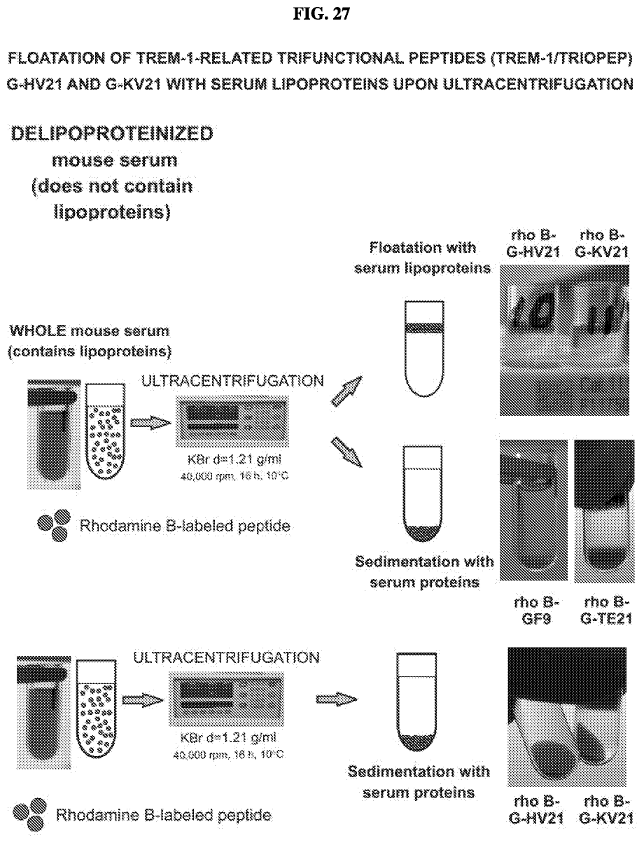

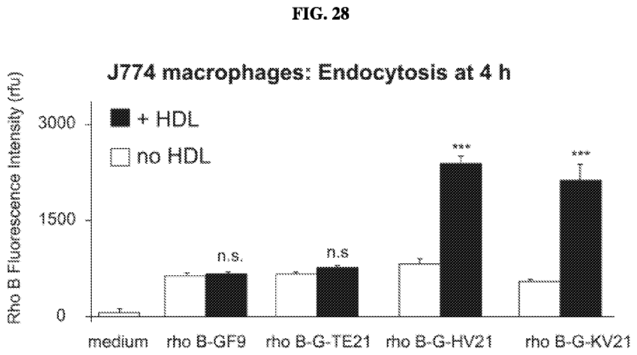

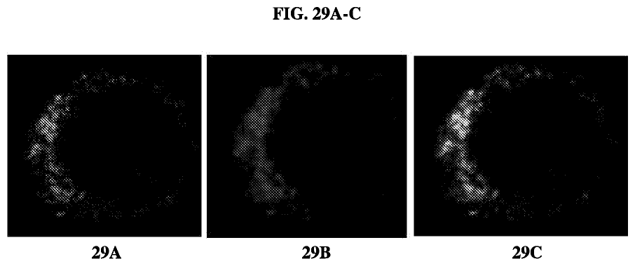

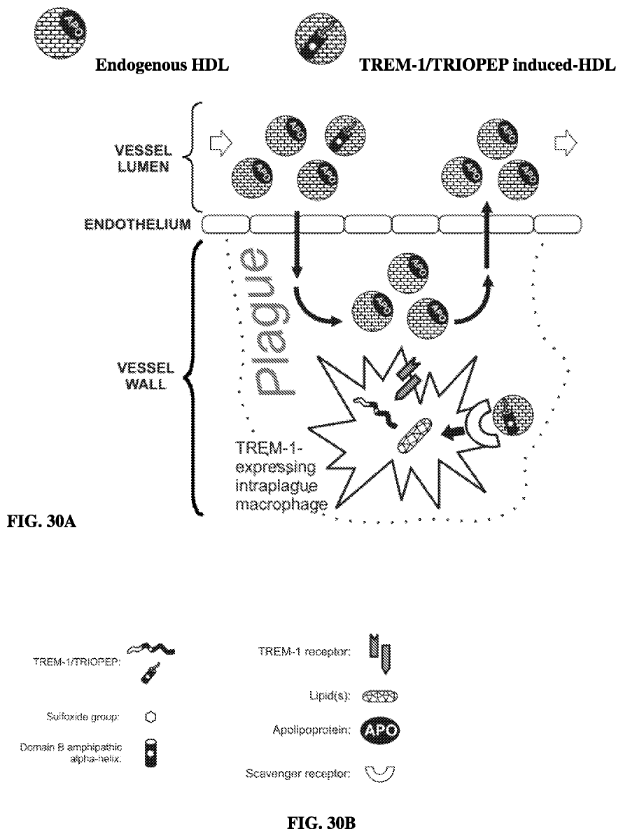

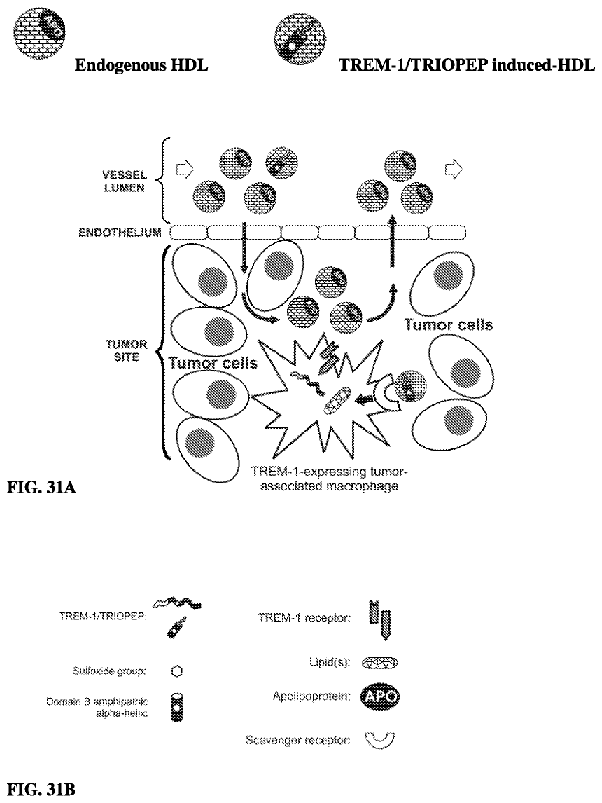

By inducing TNF-a, IL-6, MCP-1, IL-8, and granulocyte-macrophage colony-stimulating factor and inhibiting IL-10 production, TREM-1 is involved in activation and recruitment of monocytes and modulation of inflammatory responses.⁽⁴⁰⁾ Furthermore, TREM-1 expression was highly up-regulated on the surface of infiltrating monocytes and neutrophils in human tissues infected by bacteria, highlighting the importance of this receptor in these processes.⁽⁷⁾ In alcoholic hepatitis, neutrophils infiltrate the liver, inducing oxidative stress and cytotoxicity that contributes to the high mortality of the disease.⁽²⁾ We showed that these processes can be attenuated by TREM-1 inhibitors. Mechanistically, the GF9-HDL and GA/E31-HDL formulations target the liver more efficiently than peptides alone and release the TREM-1 inhibitory sequences inside the target cells where these peptides likely inhibit TREM-1 signaling by disrupting the intramembrane interactions of the TREM-1 receptor and its signaling adaptor molecule death-associated protein 12 (FIG. 27). (15-17)

It was contemplated that observed preferential endocytosis of GF9-HDL and GA/E31-HDL by macrophages and hepatic clearance of these complexes is mediated by SR recognition of putative epitopes in the modified apo A-I peptide constituents of GF9-HDL and GA/E31-HDL. (16, 17, 19) Findings described herein indicate that GF9-HDL and GA/E31-HDL are largely recognized by SR-A on macrophages (FIG. 9A-B). We also observed SR-BI-mediated uptake, which likely explains the previously observed hepatic clearance for these complexes in another animal model⁽¹⁹⁾ While these data confirm our hypothesis, future studies are needed to determine the clearance properties for GF9-HDL and GA/E31-HDL in ALD.

Further, our present study demonstrates that GF9-HDL and GA/E31-HDL exhibit not only similar macrophage uptake in vitro largely driven by SR-A (FIG. 9A1) but also similar therapeutic effect in a mouse model of ALD (FIGS. 20-21). This is in line with our previous studies where GF9-HDL and GA/E31-HDL exhibited similar therapeutic activities in cancer and arthritic mice. (16, 17) We suggest that SR-A epitopes are similarly exposed on GA31 and GE31 in GA/E31-HDL and on PA22 and PE22 in GF9-HDL, providing similar uptake of these complexes and as a result delivery of TREM-1 inhibitory GF9 peptide sequences in vivo. The use of GA/E31-HDL in the further development of effective and low-toxicity therapy for ALD is advantageous because it makes the entire manufacturing process easier and less expensive. We also suggest that the in vitro macrophage uptake assay can be potentially used to predict the outcomes for macrophage-targeted TREM-1 therapy in vivo.

In addition to attenuating inflammatory processes, the TREM-1 inhibitory formulations also ameliorated hepatocyte damage and steatosis. Serum ALT and liver triglyceride levels were both decreased in the GF9-HDL, GA/E31-HDL, and HDL-vehicle treated groups. The vehicle also had an inhibitory effect on TNF-a and MCP-1 protein levels as well as on mRNA expression of neutrophil and fibrosis markers, indicating that the HDL vehicle formulation can attenuate inflammation to a moderate extent. A previous study found evidence that HDL can protect hepatocytes from endoplasmic reticulum stress, (41) while other publications reported a scavenger function of HDL for LPS and lipoteichoic acid (42, 43) that could prevent immune cells from being activated by those molecules. (42, 43) Further, the observed moderate beneficial effect of HDL treatment alone on fatty acid oxidation markers in alcohol-exposed mice (FIG. 47A-C) is in line with data that demonstrate infusion of reconstituted HDL reduces fatty acid oxidation in patients with type 2 diabetes mellitus. (44) In human and rat plasma, apo A-I, the major protein of HDL, has been shown to inhibit lipid peroxidation. (45) These data might provide an explanation for our findings of the hepatoprotective effects of HDL.

Our study shows that TREM-1 inhibitors with HDL formulation exerted significant inhibition on early signaling events of proinflammatory processes at \hat{ }the level of cytokine mRNA and the activated p-SYK protein levels compared to the HDL vehicle alone in a mouse model of ALD. This effect presumably would be even more obvious at the protein level of cytokines in a more severe liver injury. However, in mice, the most commonly used 5-week alcohol feeding that we used resulted in moderate liver damage and minimal (25) inflammation, which is a limitation of our study. As shown on the stained liver sections, the GF9-HDL and GA/E31-HDL formulations significantly inhibited immune cell infiltration and steatosis compared to the HDL vehicle only in mice with ALD. Thus, in some emboidments, TREM-1 inhibitors, such as the trifuncitonal peptides described herein, are contemplate for administration to patients showing at least one symptom, or at risk of developing a symptom, for ALD for decreasing inflammation in liver tissue for reducing said symptom or delaying/preventing said symptom.

Materials and Methods, for Example, in Relation to Experiments Associated with Treating ALD.

### Reagents and Cells

The murine macrophage J774A.1 cell line was purchased from ATCC (Manassas, Va.). Cytochalasin D was purchased from MP Biomedicals (Solon, Ohio). Blocker of lipid transport 1 (BLT-1) was purchased from Calbiochem (Torrey Pines, Calif.). Sodium cho-late, cholesteryl oleate, fucoidan, and other chemicals were purchased from Sigma-Aldrich (St. Louis, Mo.). 1-Palmitoyl-2-oleoyl-sn-glycero-3-phosphocholine (POPC), 1,2-dimyristoyl-OT-glycero-3-phosphoeth-anolamine-N-(lissamine rhodamine B sulfonyl) (rho EEB-PE), and cholesterol were purchased from Avanti Polar Lipids (Alabaster, Ala.).

### Peptide Synthesis

The following synthetic peptides were ordered from Bachem (Torrance, Calif.): one 9-mer peptide, GFLSKSLVF (human TREM-1₂₁₃₋₂₂₁, GF9); two 22-mer methionine sulfoxidized peptides, PYLDDFQKKWQEEM(O)ELYRQKVE (H4) and PLG EEM(O)RDRARAHVDALRTHLA (H6), which correspond to human apo A-I helices 4 (apo A-1₁₂₃₋₁₄₄) and 6 (apo A-1₁₆₇₋₁₈₈), respectively; and two 31-mer methionine sulfoxidized peptides, GFLSKSLVFPYLDDFQKKWQEEM(O)ELYRQKVE (GE31) and GFLSKSLVFPLGEEM(O)RDRARAHVDALRTHLA (GA31).

### Lipopeptide Complexes

HDL-mimicking lipopeptide complexes of spherical morphology that contained either GF9 and an equimolar mixture of PE22 and PA22 (GF9-HDL) or an equimolar mixture of GA31 and GE31 (GA/E31-HDL) were synthesized using the sodium cholate dialysis procedure, purified, and characterized as described. (16-18, 22) For GF9-HDL, the initial molar ratio was 125:6:2:3:1:210, corresponding to POPC: cholesterol:cholesteryl oleate:GF9: apo A-I:sodium cholate, respectively, where apo A-I was an equimolar mixture of PE22 and PA22. For GA/E31-HDL, the initial molar ratio was 125:6:2:1:210, corresponding to POPC:cholesterol:cholesteryl oleate:GA/E31: sodium cholate, respectively, where GA/E31 was an equimolar mixture of GA31 and GE31.

In Vitro Macrophage Uptake of GF9HDL and GA/E31HDL A quantitative in vitro macrophage assay of endo-cytosis of rho B-labeled HDL-mimicking lipopeptide complexes by J774 macrophage was performed as described. (18-20) Briefly, BALB/c murine macrophage J774A.1 cells (ATCC) were cultured at 37° C. with 5% CO₂ in Dulbecco's modified Eagle's medium (Cellgro Mediatech, Manassas, Va.) with 2 mM glutamine, 100 U/mL penicillin, 0.1 mg/mL streptomycin, and 10% heat-inactivated fetal bovine serum (Cellgro Mediatech) and grown to approximately 90% confluency in 12-well tissue culture plates (Corning Costar, Corning, N.Y.). After reaching target confluency, cells were incubated for 1 hour in medium with or without fucoidan (400 ug/mL), BLT-1 ((10 μM), or cytochalasin D (40 μM). Cells were subsequently incubated for 4 hours and 22 hours at 37° C. in medium containing 2 μM of rho B-labeled GF9-HDL or GA/E31-HDL (as calculated for rho B).

Cells were washed twice using phosphate-buffered saline and lysed using Passive Lysis Buffer (Promega, Madison, Wis.). Rho B fluorescence was measured in the lysates with 544-nm excitation and 590-nm emission filters, using a Fluoroscan Ascent CF fluorescence microplate reader (Thermo Labsystems, Vantaa, Finland). Protein concentrations in the lysates were measured using Bradford reagent (Sigma-Aldrich) and an MRX microplate reader (Dynex Technologies, Chantilly, Va.) according to the manufacturer's recommended protocol.

### Animals

C57BL/6 female mice (10- to 12-week-old) were purchased from the Jackson Laboratory (Bar Harbor, Me.) and housed at the University of Massachusetts Medical School (UMMS) animal facility. Animals received humane care in accordance with protocols approved by the UMMS Institutional Animal Use and Care Committee. Mice (n=6-9/group) were acclimated to a Lieber-DeCarli liquid diet of 5% eth-anol (EtOH) (volume [vol]/vol) over a period of 1 week, then maintained on the 5% diet for 4 weeks. Pair-fed (PF) control mice were fed a calorie-matched dextran-maltose diet. Animals had unrestricted access to water throughout the entire experimental period. In treated groups, mice were intraperitone-ally treated 5 days/week with vehicle (empty HDL) or the TREM-1 inhibitory formulations GF9-HDL (2.5 mg of GF9/kg) or GA/E31-HDL (4 mg equivalent of GF9/kg) (SignaBlok, Shrewsbury, Mass.) from the first day on a 5% EtOH diet. At the end of all animal experiments, cheek blood samples were collected in serum collection tubes (BD Biosciences, San Jose, Calif.) and processed within an hour. After blood collections, mice were euthanized and liver samples were harvested and stored at −80° C. until further analysis.

Total Protein Isolation from Liver

Total protein was extracted from liver samples using radio immunoprecipitation assay buffer (BP-115; Boston BioProducts) supplemented with protease inhibitor cocktail tablets (11836153001; Roche) and Phospho Stop phosphatase inhibitor (04906837001; Roche). Cell debris was removed from cell lysates by 10 minutes centrifugation at 2,000 rpm.

### Biochemical Assays and Cytokines

Serum ALT levels were determined by the kinetic method using commercially available reagents from Teco Diagnostics (Anaheim, Calif.). Cytokine levels were measured in serum samples, and whole-liver lysates were diluted in assay diluent following the manufacturer's instructions. Specific anti-mouse enzyme-linked immunosorbent assay (ELISA) kits were used for the quantification of MCP-1, TNF-α (BioLegend Inc., San Diego, Calif.), and IL-ip (R&D Systems, Minneapolis, Minn.) levels. For normalization, the total protein concentration of the whole-liver lysate was determined using the Pierce bicinchoninic acid protein assay.

### Western Blot Analysis

Whole-liver proteins were boiled in Laemmli's buffer. Samples were resolved in 10% sodium dodecyl sulfate-polyacrylamide gel electrophoresis gel under reducing conditions, using a Tris-glycine buffer system; resolved proteins were transferred onto a nitrocellulose membrane. SYK proteins were detected by specific primary antibodies (SYK, 2712 [Cell Signaling]; phospho-SYK^{Y525/526}, ab58575 [Abeam]) followed by an appropriate secondary horseradish peroxidase-conjugated immunoglobulin G antibody from Santa Cruz Biotechnology, p-actin, detected by an ab49900 antibody (Abeam), was used as a loading control. The specific immunoreactive bands of interest were visualized by chemiluminescence (Bio-Rad Laboratories) using the Fujifilm LAS-4000 luminescent image analyzer.

RNA Extraction and Quantitative Realtime Polymerase Chain Reaction Analysis

Total RNA was extracted using the Qiagen RNeasy kit (Qiagen) according to the manufacturer's instructions with on-column deoxyribonuclease treatment. RNA was quantified using a Nanodrop 2000 spectrophotometer (Thermo Fisher Scientific), and complementary DNA synthesis was performed using the iScript Reverse Transcription Supermix (Bio-Rad Laboratories) and 1 ug total RNA. Real-time quantitative polymerase chain reaction (PCR) was performed using Bio-Rad iTaq Universal SYBR Green Supermix and a CFX96 real-time detection system (Bio-Rad Laboratories). Relative gene expression was calculated by the comparative ΔΔACt method. The expression level of target genes was normalized to the housekeeping gene 18S ribosomal RNA in each sample, and the fold change in the target gene expression among experimental groups was expressed as a ratio. Primers were synthesized by IDT, Inc.; exemplary sequences are listed in Table 1.

### Liver Histopathology

Sections of formalin-fixed paraffin-embedded liver specimens from mice were stained with hematoxylin and eosin (H&E) or F4/80 (MF48000; Thermo Fisher Scientific) and MPO (ab9535; Abeam) antibodies for immunohistochemistry (IHC). The fresh-frozen samples were stained with Oil Red O at the UMMS Diabetes and Endocrinology Research Center histology core facility.

### Statistical Analysis

Statistical analyses were performed using GraphPad Prism 7.02 (GraphPad Software Inc.). Significance levels were determined using one-way analysis of variance followed by a post-hoc test for multiple comparisons. Data are shown as mean±SEM, and differences were considered statistically significant when P≤0.05.

### REFERENCES RELATED TO SECTIONS ON ALD

1) Szabo G, Bala S, Petrasek J, Gattu A. Gut-liver axis and sensing microbes. Dig Dis 2010; 28:737-744.
2) Bautista A P. Neutrophilic infiltration in alcoholic hepatitis. Alcohol 2002; 27:17-21.
3) Szabo G, Petrasek J, Bala S. Innate immunity and alcoholic liver disease. Dig Dis 2012; 30(Suppl 1.):55-60.
4) Tessarz A S, Cerwenka A. The TREM-1/DAP12 pathway. Immunol Lett 2008; 116:111-116.
5) Arts R J, Joosten L A, Dinarello C A, Kullberg B J, van der Meer J W, Netea M G. TREM-1 interaction with the LPS/TLR4 receptor complex. Eur Cytokine Netw 2011; 22:11-14.
6) Campanholle G, Mittelsteadt K, Nakagawa S, Kobayashi A, Lin S L, Gharib S A, et al. TLR-2/TLR-4 TREM-1 signaling pathway is dispensable in inflammatory myeloid cells during sterile kidney injury. PLoS One 2013; 8:e68640.
7) Bouchon A, Facchetti F, Weigand M A, Colonna M. TREM-1 amplifies inflammation and is a crucial mediator of septic shock. Nature 2001; 410:1103-1107.
8) Zysset D, Weber B, Rihs S, Brasseit J, Freigang S, Riether C, et al. TREM-1 links dyslipidemia to inflammation and lipid deposition in atherosclerosis. Nat Commun 2016; 7:13151.
9) Schenk M, Bouchon A, Seibold F, Mueller C. TREM-1 expressing intestinal macrophages crucially amplify chronic inflammation in experimental colitis and inflammatory bowel diseases. J Clin Invest 2007; 117:3097-3106.
10) Liao R, Sun T W, Yi Y, Wu H, Li Y W, Wang F X, et al. Expression of TREM-1 in hepatic stellate cells and prognostic value in hepatitis B-related hepatocellular carcinoma. Cancer Sci 2012; 103:984-992.
11) Wu J, Li J, Salcedo R, Mivechi N F, Trinchieri G, Horuzsko A. The proinflammatory myeloid cell receptor TREM-1 controls Kupffer cell activation and development of hepatocellular carcinoma. Cancer Res 2012; 72:3977-3986.
12) Read C B, Kuijper J L, Hjorth S A, Heipel M D, Tang X, Fleetwood A J, et al. Cutting edge: identification of neutrophil PGLYRP1 as a ligand for TREM-1.J Immunol 2015; 194:1417-1421.
13) Sigalov A B. Multichain immune recognition receptor signaling: different players, same game? Trends Immunol 2004; 25:583-589.
14) Sigalov A B. Immune cell signaling: a novel mechanistic model reveals new therapeutic targets. Trends Pharmacol Sci 2006; 27:518-524.
15) Rojas M A, Shen Z T, Caldwell R B, Sigalov A B. Blockade of TREM-1 prevents vitreoretinal neovascularization in mice with oxygen-induced retinopathy. Biochim Biophys Acta Mol Basis Dis 2018; 1864:2761-2768.
16) Shen Z T, Sigalov A B. Rationally designed ligand-independent peptide inhibitors of TREM-1 ameliorate collagen-induced arthritis. J Cell Mol Med 2017; 21:2524-2534.
17) Shen Z T, Sigalov A B. Novel TREM-1 inhibitors attenuate tumor growth and prolong survival in experimental pancreatic cancer. Mol Pharm 2017; 14:4572-4582.
18) Sigalov A B. A novel ligand-independent peptide inhibitor of TREM-1 suppresses tumor growth in human lung cancer xenografts and prolongs survival of mice with lipopolysaccharide-in-duced septic shock. Int Immunopharmacol 2014; 21:208-219.
19) Shen Z T, Zheng S, Gounis M J, Sigalov A B. Diagnostic magnetic resonance imaging of atherosclerosis in apolipoprotein E knockout mouse model using macrophage-targeted gadolinium-containing synthetic lipopeptide nanoparticles. PLoS One 2015; 10:e0143453.
20) Sigalov A B. Nature-inspired nanoformulations for contrast-enhanced in vivo MR imaging of macrophages. Contrast Media Mol Imaging 2014; 9:372-382.
21) Lieber C S, DeCarli L M. The feeding of alcohol in liquid diets: two decades of applications and 1982 update. Alcohol Clin Exp Res 1982; 6:523-531.
22) Shen Z T, Sigalov A B. SARS coronavirus fusion peptide-derived sequence suppresses collagen-induced arthritis in DBA/If mice. Sci Rep 2016; 6:28672.
23) Iracheta-Vellve A, Petrasek J, Satishchandran A, Gyongyosi B, Saha B, Kodys K, et al. Inhibition of sterile danger signals, uric acid and ATP, prevents inflammasome activation and protects from alcoholic steatohepatitis in mice. J Hepatol 2015; 63:1147-1155.
24) Bukong T N, Iracheta-Vellve A, Saha B, Ambade A, Satishchandran A, Gyongyosi B, et al. Inhibition of spleen tyrosine kinase activation ameliorates inflammation, cell death, and steatosis in alcoholic liver disease. Hepatology 2016; 64:1057-1071.
25) Bertola A, Mathews S, Ki S H, Wang H, Gao B. Mouse model of chronic and binge ethanol feeding (the NIAAA model). Nat Protoc 2013; 8:627-637.
26) Ju C, Mandrekar P. Macrophages and alcohol-related liver inflammation. Alcohol Res 2015; 37:251-262.
27) Teli M R, Day C P, Burt A D, Bennett M K, James O F. Determinants of progression to cirrhosis or fibrosis in pure alcoholic fatty liver. Lancet 1995; 346:987-990.
28) Terpstra V, van Berkel T J. Scavenger receptors on liver Kupffer cells mediate the in vivo uptake of oxidatively damaged red blood cells in mice. Blood 2000; 95:2157-2163.
29) Zingg J M, Ricciarelli R, Azzi A. Scavenger receptors and modified lipoproteins: fatal attractions? ITJBMB Life 2000; 49:397-403.
30) Gu B J, Saunders B M, Jursik C, Wiley J S. The P2X7-nonmuscle myosin membrane complex regulates phagocytosis of nonop-sonized particles and bacteria by a pathway attenuated by extracellular ATP. Blood 2010; 115:1621-1631.
31) Sigola L B, Fuentes A L, Millis L M, Vapenik J, Murira A. Effects of Toll-like receptor ligands on RAW 264.7 macrophage morphology and zymosan phagocytosis. Tissue Cell 2016; 48:389-396.
32) Yu M, Romer K A, Nieland T J, Xu S, Saenz-Vash V, Penman M, et al. Exoplasmic cysteine Cys384 of the HDL receptor SR-BI is critical for its sensitivity to a small-molecule inhibitor and normal lipid transport activity. Proc Natl Acad Sci USA 2011; 108:12243-12248.
33) Dong P, Xie T, Zhou X, Hu W, Chen Y, Duan Y, et al. Induction of macrophage scavenger receptor type B I expression by tamoxifen and 4-hydroxytamoxifen. Atherosclerosis 2011; 218:435-442.
34) Tammaro A, Derive M, Gibot S, Leemans J C, Florquin S, Dessing M C. TREM-1 and its potential ligands in non-infectious diseases: from biology to clinical perspectives. Pharmacol Ther 2017; 177:81-95.
35) El Mezayen R, El Gazzar M, Seeds M C, McCall C E, Dreskin S C, Nicolls M R. Endogenous signals released from necrotic cells augment inflammatory responses to bacterial endotoxin. Immunol Lett 2007; 111:36-44.
36) Gibot S, Buonsanti C, Massin F, Romano M, Kolopp-Sarda M N, Benigni F, et al. Modulation of the triggering receptor expressed on the myeloid cell type 1 pathway in murine septic shock. Infect Immun 2006; 74:2823-2830.
37) Bala S, Marcos M, Gattu A, Catalano D, Szabo G. Acute binge drinking increases serum endotoxin and bacterial DNA levels in healthy individuals. PLoS One 2014; 9:e96864.
38) Arts R J, Joosten L A, van der Meer J W, Netea M G. TREM-1: intracellular signaling pathways and interaction with pattern recognition receptors. J Leukoc Biol 2013; 93:209-215.
39) Klesney-Tait J, Turnbull I R, Colonna M. The TREM receptor family and signal integration. Nat Immunol 2006; 7:1266-1273.
40) Bleharski J R, Kiessler V, Buonsanti C, Sieling P A, Stenger S, Colonna M, et al. A role for triggering receptor expressed on myeloid cells-1 in host defense during the early-induced and adaptive phases of the immune response. J Immunol 2003; 170:3812-3818.
41) Hong D, Li L F, Gao H C, Wang X, Li C C, Luo Y, et al. High-density lipoprotein prevents endoplasmic reticulum stress-induced downregulation of liver LOX-1 expression. PLoS One 2015; 10:e0124285.
42) Feingold K R, Grunfeld C. The role of HDL in innate immunity. J Lipid Res 2011; 52:1-3.
43) Tobias P S, Ulevitch R J. Control of lipopolysaccharide-high density lipoprotein binding by acute phase protein(s). J Immunol 1983; 131:1913-1916.
44) Drew B G, Carey A L, Natoli A K, Formosa M F, Vizi D, Reddy-Luthmoodoo M, et al. Reconstituted high-density lipoprotein infusion modulates fatty acid metabolism in patients with type 2 diabetes mellitus. J Lipid Res 2011; 52:572-581.
45) Mashima R, Yamamoto Y, Yoshimura S. Reduction of phosphatidylcholine hydroperoxide by apolipoprotein A-I: purification of the hydroperoxide-reducing proteins from human blood plasma. J Lipid Res 1998; 39:1133-1140.

Taken together, this highlights the urgent need for novel approaches to prevent, treat and/or diagnose these diseases. However, it should be noted that the techniques and compositions listed and described herein are applicable to a broad range of disease states including, but not limiting to, cardiovascular disease, bacterial infectious diseases, diabetes, and autoimmune diseases. Other features and advantages of the invention will become apparent from the following detailed description. It should be understood, however, that the detailed description and the specific examples, while indicating preferred embodiments of the invention, are given by way of illustration, because various changes and modifications within the spirit and scope of the invention will become apparent to those skilled in the art from this detailed description.

### VI. Imaging Probes.

In one embodiment, one or both amino acid domains of the peptides and compounds of the present invention are conjugated to an imaging probe. In one embodiment, the peptides and compounds of the present invention are used in combinations thereof. In one embodiment, the present invention relates to the targeted treatment, prevention and/or detection of cancer including but not limited to lung, pancreatic, breast, stomach, prostate, colon, brain and skin cancers, cancer cachexia, atherosclerosis, allergic diseases, acute radiation syndrome, inflammatory bowel disease, empyema, acute mesenteric ischemia, hemorrhagic shock, multiple sclerosis, liver diseases, autoimmune diseases, including but not limited to, atopic dermatitis, lupus, scleroderma, rheumatoid arthritis, psoriatic arthritis and other rheumatic diseases, sepsis and other inflammatory diseases or other condition involving myeloid cell activation and, more particularly, TREM receptor-mediated cell activation, including but not limited to diabetic retinopathy and retinopathy of prematurity, Alzheimer's, Parkinson's and Huntington's diseases.

VII. Exemplary Methods of Providing Synthetic (Recombinant) Lipopeptide Particles (SLPs or rHDLs) and Synthetic Peptides.

In one embodiment, the invention provides methods for making SLPs. The method comprises co-dissolving a predetermined amount of a mixture of neutral and/or charged lipids. The method further comprises drying the mixture under nitrogen. The method even further comprises co-dissolving the dried mixture with a predetermined amount of a trifunctional peptide or compound of the present invention or combinations thereof. The co-dissolving is conducted for a time period sufficient to allow the mixture to self-assemble into structures whereby particles are formed. The method further comprises isolating particles that have a size of between about 5 to about 200 nm diameter.

The lipid of the method may include PC, PE, PS, PI, PG, CL, SM, DOTAP or PA. In certain embodiments, the invention provides a method for making SLP comprising co-dissolving a predetermined amount of a mixture of neutral and/or charged lipids with a predetermined amount of cholesterol, a predetermined amount of triglycerides and/or cholesteryl ester. The method further comprises drying the mixture under nitrogen. The method even further comprises co-dissolving the dried mixture with a predetermined amount of sodium cholate and a predetermined amount of a trifunctional peptide or compound of the present invention or combinations thereof. The co-dissolving is conducted for a time period sufficient to allow the components to coalesce into particles. The method still further comprises removing sodium cholate from the mixture, and isolating particles that have a size of between about 5 to about 200 nm diameter. The lipid of the method may include PC, PE, PS, PI, PG, CL, SM, DOTAP, or PA.

In one embodiment, in the methods of the present disclosure, the peptides and compounds of the invention are pre-formulated into synthetic lipopeptide particles (SLP). In one embodiment, SLPs are discoidal in shape. In one embodiment, SLPs are spherical in shape.

While the size of the particles is preferably between 5 nm and 50 nm, the diameter may be up to 200 nm. In one embodiment, the lipid of the particles may include cholesterol, a cholesteryl ester, a phospholipid, a glycolipid, a sphingolipid, a cationic lipid, a diacylglycerol, or a triacylglycerol. And further, the phospholipid may include phosphatidylcholine (PC), phosphatidylethanolamine (PE), phosphatidylserine (PS), phosphatidylinositol (PI), phosphatidylglycerol (PG), cardiolipin (CL), sphingomyelin (SM), or phosphatidic acid (PA), and any combinations thereof.

And even further, the cationic lipid can be 1,2-dioleoyl-3-trimethylammonium-propane (DOTAP). The lipid of the synthetic nanoparticle may be polyethylene glycol(PEG)ylated. In one embodiment, lipid is conjugated to at least one imaging probe.

In certain embodiments, an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol, or a combination thereof.

In one embodiment, the imaging agent is a GBCA for MRI. In one embodiment, the imaging agent is a [⁶⁴Cu]-containing imaging probe for imaging systems such as PET imaging systems (and combined PET/CT and PET/MRI systems). In one embodiment, the peptides and compositions of the invention are used in combinations thereof. In one embodiment, the peptides and compositions of the invention are used in combinations with other anticancer therapeutic agents. In certain embodiments, the modulators and compositions described herein are incorporated into long half-life SLP. In certain embodiments, the modulators and compositions described herein may incorporate into lipopeptide particles (LP) in vivo upon administration to the individual. In certain embodiments, the peptides and compositions of the invention can cross the blood-brain barrier (BBB), blood-retinal barrier (BRB) and blood-tumor barrier (BTB). Thus, in one aspect, the invention provides for a method for suppressing tumor growth in an individual in need thereof by administering to the individual an amount of a TREM-1 inhibitor that is effective for suppressing tumor growth.

A. Discoidal SLP (dSLP).

In one embodiment, the invention provides a method for making discoidal SLP (dSLP). The method comprises co-dissolving a predetermined amount of a mixture of neutral and/or charged lipids. The method further comprises drying the mixture under nitrogen. The method even further comprises co-dissolving the dried mixture with a predetermined amount of a trifunctional peptide or compound of the present invention or combinations thereof. The co-dissolving is conducted for a time period sufficient to allow the mixture to self-assemble into structures whereby particles are formed. The method further comprises isolating particles that have a size of between about 5 to about 200 nm diameter.

B. Spherical SLP (sSLP).

In one embodiment, the invention provides a method for making spherical SLP (sSLP) comprising co-dissolving a predetermined amount of a mixture of neutral and/or charged lipids with a predetermined amount of cholesterol, a predetermined amount of triglycerides and/or cholesteryl ester. The method further comprises drying the mixture under nitrogen. The method even further comprises co-dissolving the dried mixture with a predetermined amount of sodium cholate and a predetermined amount of a trifunctional peptide or compound of the present invention or combinations thereof. The co-dissolving is conducted for a time period sufficient to allow the components to coalesce into particles. The method still further comprises removing sodium cholate from the mixture, and isolating particles that have a size of between about 5 to about 200 nm diameter.

From second prov

C. Peptides.

Synthetic peptides, including trifunctional peptides of the present invention may include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH2)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Naturally occurring residues are divided into groups based on common side chain properties:

(1) hydrophobic: norleucine, methioninc (Met), Alanine (Ala), Valine (Val), Leucine (Leu), Isoleucine (Ile), Histidine (His), Tryptophan (Trp), Tyrosine (Tyr), Phenylalanine (Phe);
(2) neutral hydrophilic: Cysteine (Cys), Serine (Ser), Threonine (Thr);
(3) acidic/negatively charged: Aspartic acid (Asp), Glutamic acid (Glu);
(4) basic: Asparagine (Asn), Glutamine (Gln), Histidine (His), Lysine (Lys), Arginine (Arg);
(5) residues that influence chain orientation: Glycine (Gly), Proline (Pro);
(6) aromatic: Tryptophan (Trp), Tyrosine (Tyr), Phenylalanine (Phe), Histidine (His);
(7) polar: Ser, Thr, Asn, Gln;
(8) basic positively charged: Arg, Lys, His; and;
(9) charged: Asp, Glu, Arg, Lys, His

Analogues may be generated by substitutional mutagenesis and retain the biological activity of the original trifunctional peptides. Examples of substitutions identified as “conservative substitutions” are shown in TABLE 1. If such substitutions result in a change not desired, then other type of substitutions, denominated “exemplary substitutions” in Table 1, or as further described herein in reference to amino acid classes, are introduced and the products screened for their capability of executing three functions.

TABLE 1
Amino acid substitutions.
Amino acid substitution

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, norleucine | Leu |

TABLE 2A
Exemplary Trifunctional Peptides and
Compositions.

| | Exemplary |
|---|---|
| ## | Trifunctional Peptides and Compositions |
| 1 | GFLSKSLVFGEEMRDRARAHV |
| 2 | GFLSKSLVFGEEM(O)RDRARAHV |
| 3 | GFLSKSLVFWQEEMELYRQKV |
| 4 | GFLSKSLVFWQEEM(O)ELYRQKV |
| 5 | GFLSRSLVFGEEMRDRARAHV |
| 6 | GFLSRSLVFGEEM(O)RDRARAHV |
| 7 | GFLSRSLVFWQEEMELYRQKV |
| 8 | GFLSRSLVFWQEEM(O)ELYRQKV |
| 9 | GLLSKSLVFGEEMRDRARAHV |
| 10 | GLLSKSLVFGEEM(O)RDRARAHV |
| 11 | GLLSKSLVFWQEEMELYRQKV |
| 12 | GLLSKSLVFWQEEM(O)ELYRQKV |
| 13 | GFLSKSLVFGEEMRDRARAHVRGD |
| 14 | GFLSKSLVFWQEEMELYRQKVRGD |
| 15 | GFLSKSLVFPLGEEMRDRARAHVDALRTHLA |
| 16 | GFLSKSLVFPYLDDFQKKWQEEMELYRQKVE |
| 17 | GFLSKSLVFPLGEEM(O)RDRARAHVDALRTHLA |
| 18 | GFLSKSLVFPYLDDFQKKWQEEM(O)ELYRQKVE |
| 19 | GFLSKSLVFPLGEEMRDRARAHVDALRTHLARGD |
| 20 | GFLSKSLVFPYLDDFQKKWQEEMELYRQKVERGD |
| 21 | [⁶⁴Cu]GFLSKSLVFGEEM(O)RDRARAHV |
| 22 | [⁶⁴Cu]GFLSKSLVFWQEEM(O)ELYRQKV |
| 23 | [⁶⁴Cu]GFLSKSLVFPLGEEM(O)RDRARAHVDALRTHLA |
| 24 | [⁶⁴Cu]GFLSKSLVFPYLDDFQKKWQEEM(O)ELYRQKVE |
| 25 | LQEEDAGEYGCMPLGEEM(O)RDRARAHVDALRTHLA |
| 26 | LQEEDAGEYGCMPYLDDFQKKWQEEM(O)ELYRQKVE |
| 27 | [⁶⁴Cu]LQEEDAGEYGCMPLGEEM(O)RDRARAHVDALRTHLA |
| 28 | [⁶⁴Cu]LQEEDAGEYGCMPYLDDFQKKWQEEM(O)ELYRQKVE |
| 29 | LQEEDAGEYGCMGEEM(O)RDRARAHV |
| 30 | LQEEDAGEYGCMWQEEM(O)ELYRQKV |
| 31 | LQVTDSGLYRCVIYHPPPLGEEM(O)RDRARAHVDALRTHLA |
| 32 | LQVTDSGLYRCVIYHPPPYLDDFQKKWQEEM(O)ELYRQKVE |
| 33 | [⁶⁴Cu]LQVTDSGLYRCVIYHPPPLGEEM(O)RDRARAHVDALRT |
| | HLA |
| 34 | [⁶⁴Cu]LQVTDSGLYRCVIYHPPPYLDDFQKKWQEEM(O)ELYRQ |
| | KVE |
| 35 | LQVTDSGLYRCVIYHPPGEEM(O)RDRARAHV |
| 36 | LQVTDSGLYRCVIYHPPWQEEM(O)ELYRQKV |
| 37 | MWKTPTLKYFPLGEEMRDRARAHVDALRTHLA |
| 38 | MWKTPTLKYFPYLDDFQKKWQEEMELYRQKVE |
| 39 | MWRTPTLRYFPLGEEMRDRARAHVDALRTHLA |
| 40 | MWRTPTLRYFPYLDDFQKKWQEEMELYRQKVE |
| 41 | [⁶⁴Cu]MWKTPTLKYFPLGEEMRDRARAHVDALRTHLA |
| 42 | [⁶⁴Cu]MWKTPTLKYFPYLDDFQKKWQEEMELYRQKVE |
| 43 | GARSMTLTVQARQLPLGEEMRDRARAHVDALRTHLA |
| 44 | GARSMTLTVQARQLPYLDDFQKKWQEEMELYRQKVE |
| 45 | [⁶⁴Cu]GARSMTLTVQARQLPLGEEMRDRARAHVDALRTHLA |
| 46 | [⁶⁴Cu]GARSMTLTVQARQLPYLDDFQKKWQEEMELYRQKVE |
| 47 | GVLRLLLFKLPLGEEMRDRARAHVDALRTHLA |
| 48 | GVLRLLLFKLPYLDDFQKKWQEEMELYRQKVE |
| 49 | [⁶⁴Cu]GVLRLLLFKLPLGEEMRDRARAHVDALRTHLA |
| 50 | [⁶⁴Cu]GVLRLLLFKLPYLDDFQKKWQEEMELYRQKVE |
| 51 | LGKATLYAVLPLGEEMRDRARAHVDALRTHLA |
| 52 | LGKATLYAVLPYLDDFQKKWQEEMELYRQKVE |
| 53 | [⁶⁴Cu]LGKATLYAVLPLGEEMRDRARAHVDALRTHLA |
| 54 | [⁶⁴Cu]LGKATLYAVLPYLDDFQKKWQEEMELYRQKVE |
| 55 | YLLDGILFIYPLGEEMRDRARAHVDALRTHLA |
| 56 | YLLDGILFIYPYLDDFQKKWQEEMELYRQKVE |
| 57 | [⁶⁴Cu]YLLDGILFIYPLGEEMRDRARAHVDALRTHLA |
| 58 | [⁶⁴Cu]YLLDGILFIYPYLDDFQKKWQEEMELYRQKVE |
| 59 | IIVTDVIATLPLGEEMRDRARAHVDALRTHLA |
| 60 | IIVTDVIATLPYLDDFQKKWQEEMELYRQKVE |
| 61 | [⁶⁴Cu]IIVTDVIATLPLGEEMRDRARAHVDALRTHLA |
| 62 | [⁶⁴Cu]IIVTDVIATLPYLDDFQKKWQEEMELYRQKVE |
| 63 | FLFAEIVSIFPLGEEMRDRARAHVDALRTHLA |
| 64 | FLFAEIVSIFPYLDDFQKKWQEEMELYRQKVE |
| 65 | [⁶⁴Cu]FLFAEIVSIFPLGEEMRDRARAHVDALRTHLA |
| 66 | [⁶⁴Cu]FLFAEIVSIFPYLDDFQKKWQEEMELYRQKVE |
| 67 | IVIVDICITGPLGEEMRDRARAHVDALRTHLA |
| 68 | IVIVDICITGPYLDDFQKKWQEEMELYRQKVE |
| 69 | [⁶⁴Cu]IVIVDICITGPLGEEMRDRARAHVDALRTHLA |
| 70 | [⁶⁴Cu]IVIVDICITGPYLDDFQKKWQEEMELYRQKVE |
| 71 | IAVAMGIRFIIMVAPLGEEMRDRARAHVDALRTHLA |
| 72 | IAVAMGIRFIIMVAPYLDDFQKKWQEEMELYRQKVE |
| 73 | GNLVRICLGAPLGEEMRDRARAHVDALRTHLA |
| 74 | GNLVRICLGAPYLDDFQKKWQEEMELYRQKVE |
| 75 | [⁶⁴Cu]GNLVRICLGAPLGEEMRDRARAHVDALRTHLA |
| 76 | [⁶⁴Cu]GNLVRICLGAPYLDDFQKKWQEEMELYRQKVE |
| 77 | VMGDLVLTVLPLGEEMRDRARAHVDALRTHLA |
| 78 | VMGDLVLTVLPYLDDFQKKWQEEMELYRQKVE |
| 79 | [⁶⁴Cu]VMGDLVLTVLPLGEEMRDRARAHVDALRTHLA |
| 80 | [⁶⁴Cu]VMGDLVLTVLPYLDDFQKKWQEEMELYRQKVE |
| 81 | LVAADAVASLPLGEEMRDRARAHVDALRTHLA |
| 82 | LVAADAVASLPYLDDFQKKWQEEMELYRQKVE |
| 83 | [⁶⁴Cu]LVAADAVASLPLGEEMRDRARAHVDALRTHLA |
| 84 | [⁶⁴Cu]LVAADAVASLPYLDDFQKKWQEEMELYRQKVE |
| 85 | SIATGMVGALLLLLVVALGIGLFMRPLGEEMRDRARAHVDALRT |
| | HLA |
| 86 | SIATGMVGALLLLLVVALGIGLFMRPYLDDFQKKWQEEMELYRQ |
| | KVE |
| 87 | DIVKLTVYDCIRRRRRRRRRPLGEEMRDRARAHVDALRTHLA |
| 88 | DIVKLTVYDCIRRRRRRRRRPYLDDFQKKWQEEMELYRQKVE |
| 89 | SLRRSSCFGGRMDRIGAQSGLGCNSFRYPLGEEMRDRARAHVDA |
| | LRTHLA |
| 90 | SLRRSSCFGGRMDRIGAQSGLGCNSFRYPYLDDFQKKWQEEMEL |
| | YRQKVE |
| 91 | PtxGFLSKSLVFPLGEEMRDRARAHVDALRTHLA |
| 92 | PtxGFLSKSLVFPYLDDFQKKWQEEMELYRQKVE |
| 93 | PtxGFLSKSLVFPLGEEM(O)RDRARAHVDALRTHLA |
| 94 | PtxGFLSKSLVFPYLDDFQKKWQEEM(O)ELYRQKVE |
| 95 | IVILLAGGFLSKSLVFSVLFAPLGEEMRDRARAHVDALRTHLA |
| 96 | IVILLAGGFLSKSLVFSVLFAPYLDDFQKKWQEEMELYRQKVE |

TABLE 2B
Exemplary Trifunctional Peptides and
Compositions.

| | Exemplary |
|---|---|
| ## | Trifunctional Peptides and Compositions |
| 1 | GFLSKSLVFPLGEEMRDRARAHVDALRTHLA |
| 2 | GFLSKSLVFPYLDDFQKKWQEEMELYRQKVE |
| 3 | GFLSKSLVFPLGEEM(O)RDRARAHVDALRTHLA |
| 4 | GFLSKSLVFPYLDDFQKKWQEEM(O)ELRQKVE |
| 5 | GFLSKSLVFPLGEEMRDRARAHVDALRTHLARGD |
| 6 | GFLSKSLVFPYLDDFQKKWQEEMELYRQKVERGD |
| 7 | [⁶⁴Cu]GFLSKSLVFPLGEEM(O)RDRARAHVDALRTHLA |
| 8 | [⁶⁴Cu]GFLSKSLVFPYLDDFQKKWQEEM(O)ELRQKVE |
| 9 | LQEEDAGEYGCMPLGEEM(O)RDRARAHVDALRTHLA |
| 10 | LQEEDAGEYGCMPYLDDFQKKWQEEM(O)ELYRQKVE |
| 11 | [⁶⁴Cu]LQEEDAGEYGCMPLGEEM(O)RDRARAHVDALRTHLA |
| 12 | [⁶⁴Cu]LQEEDAGEYGCMPYLDDFQKKWQEEM(O)ELYRQKVE |
| 13 | LQVTDSGLYRCVIYHPPPLGEEM(O)RDRARAHVDALRTHLA |
| 14 | LQVTDSGLYRCVIYHPPPYLDDFQKKWQEEM(O)ELYRQKVE |
| 15 | [⁶⁴Cu]LQVTDSGLYRCVIYHPPPLGEEM(O)RDRARAHVDALRT |
| | HLA |
| 16 | [⁶⁴Cu]LQVTDSGLYRCVIYHPPPYLDDFQKKWQEEM(O)ELYRQ |
| | KVE |
| 17 | MWKTPTLKYFPLGEEMRDRARAHVDALRTHLA |
| 18 | MWKTPTLKYFPYLDDFQKKWQEEMELYRQKVE |
| 19 | [⁶⁴Cu]MWKTPTLKYFPLGEEMRDRARAHVDALRTHLA |
| 20 | [⁶⁴Cu]MWKTPTLKYFPYLDDFQKKWQEEMELYRQKVE |
| 21 | GARSMTLTVQARQLPLGEEMRDRARAHVDALRTHLA |
| 22 | GARSMTLTVQARQLPYLDDFQKKWQEEMELYRQKVE |
| 23 | [⁶⁴Cu]GARSMTLTVQARQLPLGEEMRDRARAHVDALRTHLA |
| 24 | [⁶⁴Cu]GARSMTLTVQARQLPYLDDFQKKWQEEMELYRQKVE |
| 25 | GVLRLLLFKLPLGEEMRDRARAHVDALRTHLA |
| 26 | GVLRLLLFKLPYLDDFQKKWQEEMELYRQKVE |
| 27 | [⁶⁴Cu]GVLRLLLFKLPLGEEMRDRARAHVDALRTHLA |
| 28 | [⁶⁴Cu]GVLRLLLFKLPYLDDFQKKWQEEMELYRQKVE |
| 29 | LGKATLYAVLPLGEEMRDRARAHVDALRTHLA |
| 30 | LGKATLYAVLPYLDDFQKKWQEEMELYRQKVE |
| 31 | [⁶⁴Cu]LGKATLYAVLPLGEEMRDRARAHVDALRTHLA |
| 32 | [⁶⁴Cu]LGKATLYAVLPYLDDFQKKWQEEMELYRQKVE |
| 33 | YLLDGILFIYPLGEEMRDRARAHVDALRTHLA |
| 34 | YLLDGILFIYPYLDDFQKKWQEEMELYRQKVE |
| 35 | [⁶⁴Cu]YLLDGILFIYPLGEEMRDRARAHVDALRTHLA |
| 36 | [⁶⁴Cu]YLLDGILFIYPYLDDFQKKWQEEMELYRQKVE |
| 37 | IIVTDVIATLPLGEEMRDRARAHVDALRTHLA |
| 38 | IIVTDVIATLPYLDDFQKKWQEEMELYRQKVE |
| 39 | [⁶⁴Cu]IIVTDVIATLPLGEEMRDRARAHVDALRTHLA |
| 40 | [⁶⁴Cu]IIVTDVIATLPYLDDFQKKWQEEMELYRQKVE |
| 41 | FLFAEIVSIFPLGEEMRDRARAHVDALRTHLA |
| 42 | FLFAEIVSIFPYLDDFQKKWQEEMELYRQKVE |
| 43 | [⁶⁴Cu]FLFAEIVSIFPLGEEMRDRARAHVDALRTHLA |
| 44 | [⁶⁴Cu]FLFAEIVSIFPYLDDFQKKWQEEMELYRQKVE |
| 45 | IVIVDICITGPLGEEMRDRARAHVDALRTHLA |
| 46 | IVIVDICITGPYLDDFQKKWQEEMELYRQKVE |
| 47 | [⁶⁴Cu]IVIVDICITGPLGEEMRDRARAHVDALRTHLA |
| 48 | [⁶⁴Cu]IVIVDICITGPYLDDFQKKWQEEMELYRQKVE |
| 49 | IAVAMGIRFIIMVAPLGEEMRDRARAHVDALRTHLA |
| 50 | IAVAMGIRFIIMVAPYLDDFQKKWQEEMELYRQKVE |
| 51 | GNLVRICLGAPLGEEMRDRARAHVDALRTHLA |
| 52 | GNLVRICLGAPYLDDFQKKWQEEMELYRQKVE |
| 53 | [⁶⁴Cu]GNLVRICLGAPLGEEMRDRARAHVDALRTHLA |
| 54 | [⁶⁴Cu]GNLVRICLGAPYLDDFQKKWQEEMELYRQKVE |
| 55 | VMGDLVLTVLPLGEEMRDRARAHVDALRTHLA |
| 56 | VMGDLVLTVLPYLDDFQKKWQEEMELYRQKVE |
| 57 | [⁶⁴Cu]VMGDLVLTVLPLGEEMRDRARAHVDALRTHLA |
| 58 | [⁶⁴Cu]VMGDLVLTVLPYLDDFQKKWQEEMELYRQKVE |
| 59 | LVAADAVASLPLGEEMRDRARAHVDALRTHLA |
| 60 | LVAADAVASLPYLDDFQKKWQEEMELYRQKVE |
| 61 | [⁶⁴Cu]LVAADAVASLPLGEEMRDRARAHVDALRTHLA |
| 62 | [⁶⁴Cu]LVAADAVASLPYLDDFQKKWQEEMELYRQKVE |
| 63 | SIATGMVGALLLLLVVALGIGLFMRPLGEEMRDRARAHVDALRT |
| | HLA |
| 64 | SIATGMVGALLLLLVVALGIGLFMRPYLDDFQKKWQEEMELYRQ |
| | KVE |
| 65 | DIVKLTVYDCIRRRRRRRRRPLGEEMRDRARAHVDALRTHLA |
| 66 | DIVKLTVYDCIRRRRRRRRRPYLDDFQKKWQEEMELYRQKVE |
| 67 | SLRRSSCFGGRMDRIGAQSGLGCNSFRYPLGEEMRDRARAHVDA |
| | LRTHLA |
| 68 | SLRRSSCFGGRMDRIGAQSGLGCNSFRYPYLDDFQKKWQEEMEL |
| | YRQKVE |
| 69 | Ptx-GFLSKSLVFPLGEEMRDRARAHVDALRTHLA |
| 70 | Ptx-GFLSKSLVFPYLDDFQKKWQEEMELYRQKVE |
| 71 | Ptx-GFLSKSLVFPLGEEM(O)RDRARAHVDALRTHLA |
| 72 | Ptx-GFLSKSLVFPYLDDFQKKWQEEM(O)ELRQKVE |
| 73 | IVILLAGGFLSKSLVFSVLFAPLGEEMRDRARAHVDALRTHLA |
| 74 | IVILLAGGFLSKSLVFSVLFAPYLDDFQKKWQEEMELYRQKVE |

### TREM-1 Inhibitory Trifunctional SCHOOL Peptides

In certain embodiments, the present invention relates to amphipathic TREM-1 inhibitory trifunctional peptides and therapeutic compositions comprising such trifunctional peptides for use in treating cancer in combination with other cancer therapies. In one embodiment, these peptides may possess the antitumor activity. In one embodiment, these peptides may not possess the antitumor activity.

In some embodiments, each trifunctional peptide is capable of at least three functions: 1) mediating formation of naturally long half-life lipopeptide/lipoprotein particles upon interaction with lipoproteins, 2) facilitation of the targeted delivery to cells of interest and/or sites of disease, and 3) treatment, prevention, and/or detection of a disease or condition. In some embodiments, each trifunctional peptide is capable of at least three functions: 1) mediating the self-assembly of naturally long half-life lipopeptide particles upon binding to lipid or lipid mixtures, 2) facilitation of the targeted delivery to cells of interest and/or sites of disease, and 3) treatment, prevention, and/or detection of a disease or condition. In certain embodiments, the present invention relates to amphipathic trifunctional peptides consisting of two amino acid domains, wherein upon interaction with plasma lipoproteins, one amino acid domain mediates formation of naturally long half-life lipopeptide/lipoprotein particles and targets these particles to macrophages, whereas the other amino acid domain inhibits the TREM-1/DAP-12 receptor signaling complex expressed on myeloid cells including but not limited to, macrophages.

In one embodiment, the TREM-1 inhibitory trifunctional SCHOOL peptides (TRIOPEPs) of the present invention form self-assembling SLP in vitro. In one embodiment, TRIOPEPs are incorporated into self-assembled nanosized SLP of discoidal or spherical morphology (dSLP and sSLP, respectively) that contain apo A-I peptide fragments comprising 22 amino acid residue-long peptide sequences of the apo A-I helix 4 and/or helix 6. In one embodiment, the TREM-1 inhibitory trifunctional SCHOOL peptides described herein form naturally long half life lipopeptide particles in vivo. In certain embodiments, the present invention relates to peptides consisting of two amino acid domains, wherein upon binding to lipid or lipid mixtures, one amino acid domain assists in the self-assembly of naturally long half-life lipopeptide particles and targets these particles to macrophages, whereas another amino acid domain inhibits TREM-1/DAP-12 receptor complex expressed on macrophages.

In some embodiments of the present inventions, TABLE 3 presents a list of the peptides and therapeutic compositions that includes, but is not limited to the trifunctional SCHOOL peptide-based TREM-1 inhibitors and therapeutic compositions that can be used in order to treat tumors in combinations with other cancer therapies or to predict response of the subject to the treatment by using the modulators of TREM-1/DAP-12 signaling pathway in combination-therapy regiment.

Exemplary TREM-1 inhibitory trifunctional SCHOOL peptides include but are not limited to, 31 amino acid-long peptide TREM-1 inhibitory peptides GA31 (GFLSKSLVFPLGEEM(O)RDRARAHVDALRTHLA, M(O), methionine sulfoxide) (SEQ ID NO. 26) and GE31 (GFLSKSLVFPYLDDFQKKWQEEM(O)ELYRQKVE, M(O), methionine sulfoxide) (SEQ ID NO. 27). In one embodiment, methionine residues of the peptides GE31 (GFLSKSLVFPYLDDFQKKWQEEMELYRQKVE) (SEQ ID NO. 25) and GA31 (GFLSKSLVFPLGEEMRDRARAHVDALRTHLA) (SEQ ID NO. 24) are unmodified. See TABLE 3.

In one embodiment, any or both the domains comprise minimal biologically active amino acid sequence. In one embodiment, the peptide variant comprises a cyclic peptide sequence. In one embodiment, the peptide variant comprises a disulfide-linked dimer. In one embodiment, the peptide variant includes amino acids selected from the group of natural and unnatural amino acids including, but not limited to, L-amino acids, or D-amino acids.

In one embodiment, one or both amino acid domains of the peptides and compounds of the present invention are conjugated to a drug compound (TA). In one embodiment, TA is selected from the group including, but not limited to, anticancer, antibacterial, antiviral, autoimmune, anti-inflammatory and cardiovascular agents, antioxidants, and therapeutic peptides. In one embodiment, the TA is a hydrophobic therapeutic agent. The TA may also be selected from the group comprising paclitaxel, valrubicin, doxorubicin, taxotere, campotechin, etoposide, and any combination thereof.

In one embodiment, one or both amino acid domains of the peptides and compounds of the present invention are conjugated to an imaging probe. In one embodiment, the imaging agent is GBCA for MRI. In one embodiment, the imaging agent is a [⁶⁴Cu]-containing imaging probe for imaging systems such as a PET imaging system and combined PET/CT and PET/MRI systems.

In one embodiment, an imaging probe and/or an additional TA is conjugated to any or both of the domains. In one embodiment, the peptides and compounds of the present invention are used in combinations thereof.

### Embodiments of TREM-1 Inhibitory SCHOOL Peptides.

Normal transmembrane interactions between the TREM-1 and the DAP-12 dimer forming a functional TREM-1/DAP-12 receptor complex comprise positively charged lysine amino acid within the TREM-1 transmembrane portion and negatively charged aspartic acid pairs in a DAP-12 dimer, thereby allowing subunit association (See FIG. 49).

In one embodiment, the simplest TREM-1 inhibitory SCHOOL agents would be synthetic peptides and their variants (SCHOOL peptides) that correspond to the TREM-1 and/or DAP-12 transmembrane domains or their functionally important minimal protein sequences as disclosed in U.S. Pat. Nos. 8,513,185, 9,981,004 and US 20190117725. Although it is not necessary to understand the mechanism of an invention, it is believed that interactions between a lysine residue of SCHOOL peptides that correspond to the TREM-1 transmembrane domain or its functionally important minimal protein sequence and an aspartic acid residue of a DAP-12 dimer disrupt the interactions between TREM-1 and DAP-12 in the membrane, thereby “disconnecting” TREM-1 and resulting in a non-functioning receptor. Accordingly, it is believed that interactions between an aspartic residue of SCHOOL peptides that correspond to the DAP-12 transmembrane domain or its functionally important minimal protein sequence and lysine amino acid residue of the TREM-1 transmembrane domain disrupt the interactions between DAP-12 and TREM-1 in the membrane, thereby “disconnecting” DAP-12 and resulting in a non-functioning receptor. These peptide variants and compositions possess the advantages typically associated with a fully synthetic material and yet possess certain desirable features of materials derived from natural sources.

In some embodiments of the present inventions, TABLE 3 presents a list of the peptides and therapeutic compositions that includes, but is not limited to the SCHOOL peptide-based TREM-1 inhibitors and their variants that can be designed as disclosed in U.S. Pat. Nos. 8,513,185, 9,981,004 and US 20190117725 and used in order to treat tumors in combinations with other cancer therapies or to predict response of the subject to the treatment by using the modulators of TREM-1/DAP-12 signaling pathway in combination-therapy regiment.

In some embodiments, the SCHOOL peptides and their variants that inhibit TREM-1 transmembrane signaling can be used in a free form. Exemplary TREM-1 inhibitory SCHOOL peptides include but are not limited to, a 9 amino acid-long peptide TREM-1 inhibitory peptide GF9 (GFLSKSLVF) disclosed in U.S. Pat. Nos. 8,513,185, 9,981,004 and US 20190117725 and described in (Sigalov 2014, Shen and Sigalov 2017, Shen and Sigalov 2017, Rojas et al. 2018). Although it is not necessary to understand the mechanism of an invention, it is believed that free SCHOOL peptide self-inserts into the cell membrane from outside the cell, co-localizes with TREM-1/DAP-12 receptor complex and disrupts the protein-protein interactions between TREM-1 and DAP-12, thereby resulting in a non-functional receptor complex that does not provide TREM-1 transmembrane signaling upon binding to a putative TREM-1 ligand(s) (See FIG. 49, Route 1). In one embodiment, FIG. 50 demonstrates colocalization of GF9 with the TREM-1 in the cell membrane. These peptide variants and compositions possess the advantages typically associated with a fully synthetic material and yet possess certain desirable features of materials derived from natural sources.

As described in (Vlieghe et al. 2010, Lau et al. 2018), the main limitations generally attributed to therapeutic peptides are: a short half-life because of their rapid degradation by proteolytic enzymes of the digestive system and blood plasma; rapid removal from the circulation by the liver (hepatic clearance) and kidneys (renal clearance); poor ability to cross physiological barriers because of their general hydrophilicity; high conformational flexibility, resulting sometimes in a lack of selectivity involving interactions with different receptors/targets (poor specific biodistribution), causing activation of several targets and leading to side effects; eventual risk of immunogenic effects; and high synthetic and production costs (the production cost of a 5000 Da molecular mass peptide exceeds the production cost of a 500 Da molecular mass small molecule by more than 10-fold but clearly not 100-fold).

In some embodiments of the present invention, the SCHOOL peptides and their variants that inhibit TREM-1 transmembrane signaling can be formulated into self-assembling SLP of discoidal (sSLP) or spherical (sSLP) shape that mimic human naturally long half-life high density lipoproteins (HDL) and are disclosed in US 20130045161 and US 20110256224 and described in (Sigalov 2014, Shen and Sigalov 2017, Shen and Sigalov 2017, Rojas et al. 2018, Tornai et al. 2019). Although it is not necessary to understand the mechanism of an invention, it is believed that these particles provide targeted delivery of the incorporated SCHOOL peptides to target cells and increase half life of these peptides in circulation. In some embodiments, these SLP contain the modified amphipathic apolipoprotein A-I peptide fragments that not only assist in the self-assembly of SLP but also provide targeted delivery of these particles to target cells in vitro and in vivo. In some embodiments, the modification represents a sulfoxidation of methionine amino acid residue in the apo A-I peptide sequence.

In one embodiment, FIG. 49 presents a schematic representation of targeted delivery of the TREM-1 modulatory SCHOOL peptides by SLP to myeloid cells including but not limited to, macrophages including TAMs. Although it is not necessary to understand the mechanism of an invention, it is believed that SLP that contain TREM-1 modulatory SCHOOL peptides (exemplary shown for GF9) are endocytosed by macrophages through scavenger receptor(s), and then release the incorporated SCHOOL peptide, which self-inserts into the cell membrane from inside the cell, co-localizes with TREM-1/DAP-12 receptor complex and disrupts the protein-protein interactions between TREM-1 and DAP-12, thereby resulting in a non-functional receptor complex that does not induce TREM-1 transmembrane signaling upon binding to a putative TREM-1 ligand(s) (See FIG. 49, Route 2).

### Modulators of TREM-1/DAP-12 Signaling Pathway.

Modulators (inhibitors) of TREM-1/DAP-12 signaling pathway can be nonexclusively divided into two major categories: those that inhibit TREM-1 transmembrane signaling by blocking binding of TREM-1 to its ligand(s) (type I inhibitors; See FIG. 49) and those that employ a ligand binding-independent mechanism of action and modulate (inhibit) TREM-1-mediated transmembrane signaling by disrupting protein-protein interactions between TREM-1 and DAP-12 in the cell membrane (type II inhibitors; See FIG. 50). Type I inhibitors can be, in turn, subdivided into two subtypes: those that bind to TREM-1 (type Ia inhibitors) and those that bind to TREM-1 ligand(s) (type Ib inhibitors).

### Type I TREM-1 Inhibitors.

In one embodiment, exemplary TREM-1 type I inhibitors include but not limited to, antagonistic (blocking, inhibiting) anti-TREM-1 antibodies and/or their fragments such as antibodies that block and inhibit TREM-1 disclosed in U.S. Pat. Nos. 9,000,127 and 9,550,830 and described in (Brynjolfsson et al. 2016). These TREM-1 inhibitors are believed to block binding of TREM-1 to its ligand(s) by binding to the extracellular domain of TREM-1 (type Ia inhibitors, See FIG. 49).

In one embodiment, exemplary TREM-1 type I inhibitors include but not limited to, synthetic peptides derived from a part of the extracellular domain of either TREM-1 such as P1, P3 and LP17 peptides disclosed in US 20160193288, US 20150232531, U.S. Pat. Nos. 8,013,836 and 9,273,111 and described in (Gibot et al. 2004, Gibot et al. 2006) or the TREM-like transcript-1 (TLT-1) such as LR17 and LR12 peptides disclosed in US 20160193288, US 20160015773, US 20150232531, U.S. Pat. Nos. 9,255,136; 9,657,081 and 9,815,883 and described in (Derive et al. 2012). These TREM-1 inhibitors are believed to act as an endogenous decoy receptor (type Ib inhibitors, See FIG. 1) by binding TREM-1 ligands and preventing their engagement to membrane-bound TREM-1 (Pelham et al. 2014).

In some embodiments of the present invention, the TREM-1 type I inhibitors can be used in order to treat tumors in combinations with other cancer therapies or to predict response of the subject to the treatment by using the modulators of TREM-1/DAP-12 signaling pathway in combination-therapy regiment.

In some embodiments of the present inventions, TABLE 3 presents a list of the peptides and peptide analogues that includes, but is not limited to the TREM-1 type Ib peptide inhibitors and their variants that can be designed as disclosed in US 20160193288, US 20150232531, U.S. Pat. Nos. 8,013,836, 9,273,111, US 20160015773, U.S. Pat. Nos. 9,255,136; 9,657,081 and 9,815,883 and described in (Gibot et al. 2004, Gibot et al. 2006, Derive et al. 2012) and used in order to treat tumors in combinations with other cancer therapies.

### Type II TREM-1 Inhibitors.

Application of the Signaling Chain HOmoOLigomerization (SCHOOL) model of receptor signaling described in (Sigalov et al. 2004, Sigalov 2004, Sigalov 2006, Sigalov 2018) to the transmembrane signal transduction mediated by a TREM-1 receptor suggested that an inhibition of TREM-1/DAP-12 signaling may be achieved by using transmembrane-targeted agents (SCHOOL agents) which specifically disrupt interactions between TREM-1 and DAP-12 subunits in the cell membrane (See FIG. 2), thereby disconnecting TREM-1 and DAP-12 and resulting in a non-functioning TREM-1/DAP-12 receptor complex.

In some embodiments of the present invention, the TREM-1 type II inhibitors can be used in order to treat tumors in combinations with other cancer therapies or to predict response of the subject to the treatment by using the modulators of TREM-1/DAP-12 signaling pathway in combination-therapy regiment.

As described in (Tammaro et al. 2017), although TREM-1 appears to be activated by damage associated molecular patterns (DAMPs) that are shared by other pattern recognition receptors (PRRs), no TREM-1 specific (endogenous) ligand has been discovered to date. It is unknown why these ligands, specifically, share TREM-1 activation. Neither it is known what they have in common, but this information could certainly be of use in the determination of new specific ligands. This makes ligand binding-independent type II TREM-1 inhibitors advantageous compared to type I inhibitors that attempt to block binding TREM-1 to its yet unknown ligand(s).

In some embodiments, type II TREM-1 inhibitors include but are not limited to, the TREM-1 inhibitory SCHOOL peptides. The preferred peptides and compositions of the present invention comprise the TREM-1 modulatory peptide sequences designed using the SCHOOL model of TREM-1 signaling and capable of modulating TREM-1 receptor expressed on myeloid cells as disclosed in U.S. Pat. Nos. 8,513,185 and 9,981,004 and described in (Sigalov 2010, Shen and Sigalov 2017).

Listed below in TABLE 2 are reported transmembrane sequences of TREM-1 and DAP-12 in a number of species. These regions are highly conserved and the substitutions between species are very conservative. This suggests a functional role for the transmembrane regions of both, TREM-1 and DAP-12, constituents of the complex. These regions strongly interact between themselves, thus maintaining the integrity of the TREM-1/DAP-12 receptor signaling complex in resting cells. These transmembrane domains are short and should be easily mimicked by synthetic peptides and compounds. In some embodiments, synthetic peptides and compounds are contemplated that may provide successful treatment options in the clinical setting.

TABLE 2C
Sequence comparison of TREM-1 and
DAP-12 transmembrane regions (accession
codes are given in parenthesis).

| | SEQUENCE | |
|---|---|---|
| SPECIES | TREM-1 | DAP-12 |
| HUMAN | IVILLAGGFLSKSLVFSVLFA | GVLAGIVMGDLVLTVLIALAV |
| | (Q9NP99) | (O43914) |
| MOUSE | VTISVICGLLSKSLVFIILFI | GVLAGIVLGDLVLTLLIALAV |
| | (Q9JKE2) | (O54885) |
| BOVIN | IIIPAACGLLSKTLVFIGLFA | GVLAGIVLGDLMLTLLIALAV |
| | (Q6QUN5) | (Q95J79) |
| SHEEP | not known | GVLAGIVLGDLMLTLLIALAV |
| | | (Q95KS5) |
| RAT | not known | GVLAGIVLGDLVLTLLIALAV |
| | | (Q6X9T7) |
| PIG | ILPAVCGLLSKSLVFIVLFVV | GILAGIVLGDLVLTLLIALAV |
| | (Q6TYI6) | (Q9TU45) |

CLUSTAL W 2.0 multiple sequence alignment:

| | | | | |
|---|---|---|---|---|
| HUMAN | IVILLAGGFLSKSLVFSVLFA- | 21 | GVLAGIVMGDLVLTVLIALAV | 21 |
| MOUSE | VTISVICGLLSKSLVFIILFI- | 21 | GVLAGIVLGDLVLTLLIALAV | 21 |
| BOVIN | IIIPAACGLLSKTLVFIGLFA- | 21 | GVLAGIVLGDLMLTLLIALAV | 21 |
| SHEEP | — | | GVLAGIVLGDLMLTLLIALAV | 21 |
| RAT | — | | GVLAGIVLGDLVLTLLIALAV | 21 |
| PIG | -ILPAVCGLLSKSLVFIVLFVV | 21 | GILAGIVLGDLVLTLLIALAV | 21 |
| | : *:***:*** ** | | *:*****:***:**:****** | |

TABLE 3
Exemplary TREM-1/DAP-12 Pathway Modulatory
Peptide Sequences and Compositions.

| | Exemplary TREM-1/DAP-12 Pathway Modulatory |
|---|---|
| ## | Peptide Sequences and Compositions |
| 1 | IVILLAGGFLSKSLVFSVLFA |
| 2 | GFLSKSLVF |
| 3 | (GFLSKSLVF)₂ |
| 4 | GLLSKSLVF |
| 5 | GVLAGIVMGDLVLTVLIALAV |
| 6 | GIVMGDLVLT |
| 7 | IVMGDLVLT |
| 8 | LQEEDAGEYGCM |
| 9 | LQVTDSGLYRCVIYHPP |
| 10 | GFLSKSLVFGEEMRDRARAHV |
| 11 | GFLSKSLVFGEEM(O)RDRARAHV |
| 12 | GFLSKSLVFWQEEMELYRQKV |
| 13 | GFLSKSLVFWQEEM(O)ELYRQKV |
| 14 | GFLSRSLVFGEEMRDRARAHV |
| 15 | GFLSRSLVFGEEM(O)RDRARAHV |
| 16 | GFLSRSLVFWQEEMELYRQKV |
| 17 | GFLSRSLVFWQEEM(O)ELYRQKV |
| 18 | GLLSKSLVFGEEMRDRARAHV |
| 19 | GLLSKSLVFGEEM(O)RDRARAHV |
| 20 | GLLSKSLVFWQEEMELYRQKV |
| 21 | GLLSKSLVFWQEEM(O)ELYRQKV |
| 22 | GFLSKSLVFGEEMRDRARAHVRGD |
| 23 | GFLSKSLVFWQEEMELYRQKVRGD |
| 24 | GFLSKSLVFPLGEEMRDRARAHVDALRTHLA |
| 25 | GFLSKSLVFPYLDDFQKKWQEEMELYRQKVE |
| 26 | GFLSKSLVFPLGEEM(O)RDRARAHVDALRTHLA |
| 27 | GFLSKSLVFPYLDDFQKKWQEEM(O)ELYRQKVE |
| 28 | GFLSKSLVFPLGEEMRDRARAHVDALRTHLARGD |
| 29 | GFLSKSLVFPYLDDFQKKWQEEMELYRQKVERGD |
| 30 | [⁶⁴Cu]GFLSKSLVFGEEM(O)RDRARAHV |
| 31 | [⁶⁴Cu]GFLSKSLVFWQEEM(O)ELYRQKV |
| 32 | GFLSKSLVFPLGEEMRDRARAHVDALRTHLA |
| 33 | GFLSKSLVFPYLDDFQKKWQEEMELYRQKVE |
| 34 | GFLSKSLVFPLGEEM(O)RDRARAHVDALRTHLA |
| 35 | GFLSKSLVFPYLDDFQKKWQEEM(O)ELRQKVE |
| 36 | GFLSKSLVFPLGEEMRDRARAHVDALRTHLARGD |
| 37 | GFLSKSLVFPYLDDFQKKWQEEMELYRQKVERGD |
| 38 | [⁶⁴Cu]GFLSKSLVFPLGEEM(O)RDRARAHVDALRTHLA |
| 39 | [⁶⁴Cu]GFLSKSLVFPYLDDFQKKWQEEM(O)ELRQKVE |
| 40 | LQEEDAGEYGCMPLGEEM(O)RDRARAHVDALRTHLA |
| 41 | LQEEDAGEYGCMPYLDDFQKKWQEEM(O)ELYRQKVE |
| 42 | [⁶⁴Cu]LQEEDAGEYGCMPLGEEM(O)RDRARAHVDALRTHLA |
| 43 | [⁶⁴Cu]LQEEDAGEYGCMPYLDDFQKKWQEEM(O)ELYRQKVE |
| 44 | LQVTDSGLYRCVIYHPPPLGEEM(O)RDRARAHVDALRTHLA |
| 45 | LQVTDSGLYRCVIYHPPPYLDDFQKKWQEEM(O)ELYRQKVE |
| 46 | [⁶⁴Cu]LQVTDSGLYRCVIYHPPPLGEEM(O)RDRARAHVDAL |
| | RTHLA |
| 47 | [⁶⁴Cu]LQVTDSGLYRCVIYHPPPYLDDFQKKWQEEM(O)ELY |
| | RQKVE |
| 48 | LQEEDAGEYGCMGEEM(O)RDRARAHV |
| 49 | LQEEDAGEYGCMWQEEM(O)ELYRQKV |
| 50 | IIVTDVIATLPLGEEMRDRARAHVDALRTHLA |
| 51 | IIVTDVIATLPYLDDFQKKWQEEMELYRQKVE |
| 52 | [⁶⁴Cu]IIVTDVIATLPLGEEMRDRARAHVDALRTHLA |
| 53 | [⁶⁴Cu]IIVTDVIATLPYLDDFQKKWQEEMELYRQKVE |
| 54 | IIVTDVIATLPLGEEM(O)RDRARAHVDALRTHLA |
| 55 | IIVTDVIATLPYLDDFQKKWQEEM(O)ELYRQKVE |
| 56 | [⁶⁴Cu]IIVTDVIATLPLGEEM(O)RDRARAHVDALRTHLA |
| 57 | [⁶⁴Cu]IIVTDVIATLPYLDDFQKKWQEEM(O)ELYRQKVE |
| 58 | PtxGFLSKSLVFPLGEEMRDRARAHVDALRTHLA |
| 59 | PtxGFLSKSLVFPYLDDFQKKWQEEMELYRQKVE |
| 60 | PtxGFLSKSLVFPLGEEM(O)RDRARAHVDALRTHLA |
| 61 | PtxGFLSKSLVFPYLDDFQKKWQEEM(O)ELYRQKVE |
| 62 | IVILLAGGFLSKSLVFSVLFAPLGEEMRDRARAHVDALRTHL |
| | A |
| 63 | IVILLAGGFLSKSLVFSVLFAPYLDDFQKKWQEEMELYRQKV |
| | E |
| 64 | IVILLAGGFLSKSLVFSVLFAPLGEEM(O)RDRARAHVDALR |
| | THLA |
| 65 | IVILLAGGFLSKSLVFSVLFAPYLDDFQKKWQEEM(O)ELYR |
| | QKVE |
| 1 | IVILLAGGFLSKSLVFSVLFA |
| 2 | GFLSKSLVF |
| 3 | (GFLSKSLVF)₂ |
| 4 | GLLSKSLVF |
| 5 | GVLAGIVMGDLVLTVLIALAV |
| 6 | GIVMGDLVLT |
| 7 | IVMGDLVLT |
| 8 | LQEEDAGEYGCM |
| 9 | LQVTDSGLYRCVIYHPP |
| 10 | GFLSKSLVFGEEMRDRARAHV |
| 11 | GFLSKSLVFGEEM(O)RDRARAHV |
| 12 | GFLSKSLVFWQEEMELYRQKV |
| 13 | GFLSKSLVFWQEEM(O)ELYRQKV |
| 14 | GFLSRSLVFGEEMRDRARAHV |
| 15 | GFLSRSLVFGEEM(O)RDRARAHV |
| 16 | GFLSRSLVFWQEEMELYRQKV |
| 17 | GFLSRSLVFWQEEM(O)ELYRQKV |
| 18 | GLLSKSLVFGEEMRDRARAHV |
| 19 | GLLSKSLVFGEEM(O)RDRARAHV |
| 20 | GLLSKSLVFWQEEMELYRQKV |
| 21 | GLLSKSLVFWQEEM(O)ELYRQKV |
| 22 | GFLSKSLVFGEEMRDRARAHVRGD |
| 23 | GFLSKSLVFWQEEMELYRQKVRGD |
| 24 | GFLSKSLVFPLGEEMRDRARAHVDALRTHLA |
| 25 | GFLSKSLVFPYLDDFQKKWQEEMELYRQKVE |
| 26 | GFLSKSLVFPLGEEM(O)RDRARAHVDALRTHLA |
| 27 | GFLSKSLVFPYLDDFQKKWQEEM(O)ELYRQKVE |
| 28 | GFLSKSLVFPLGEEMRDRARAHVDALRTHLARGD |
| 29 | GFLSKSLVFPYLDDFQKKWQEEMELYRQKVERGD |
| 30 | [⁶⁴Cu]GFLSKSLVFGEEM(O)RDRARAHV |
| 31 | [⁶⁴Cu]GFLSKSLVFWQEEM(O)ELYRQKV |
| 32 | GFLSKSLVFPLGEEMRDRARAHVDALRTHLA |
| 33 | GFLSKSLVFPYLDDFQKKWQEEMELYRQKVE |
| 34 | GFLSKSLVFPLGEEM(O)RDRARAHVDALRTHLA |
| 35 | GFLSKSLVFPYLDDFQKKWQEEM(O)ELRQKVE |
| 36 | GFLSKSLVFPLGEEMRDRARAHVDALRTHLARGD |
| 37 | GFLSKSLVFPYLDDFQKKWQEEMELYRQKVERGD |
| 38 | [⁶⁴Cu]GFLSKSLVFPLGEEM(O)RDRARAHVDALRTHLA |
| 39 | [⁶⁴Cu]GFLSKSLVFPYLDDFQKKWQEEM(O)ELRQKVE |
| 40 | LQEEDAGEYGCMPLGEEM(O)RDRARAHVDALRTHLA |
| 41 | LQEEDAGEYGCMPYLDDFQKKWQEEM(O)ELYRQKVE |
| 42 | [⁶⁴Cu]LQEEDAGEYGCMPLGEEM(O)RDRARAHVDALRTHLA |
| 43 | [⁶⁴Cu]LQEEDAGEYGCMPYLDDFQKKWQEEM(O)ELYRQKVE |
| 44 | LQVTDSGLYRCVIYHPPPLGEEM(O)RDRARAHVDALRTHLA |
| 45 | LQVTDSGLYRCVIYHPPPYLDDFQKKWQEEM(O)ELYRQKVE |
| 46 | [⁶⁴Cu]LQVTDSGLYRCVIYHPPPLGEEM(O)RDRARAHVDAL |
| | RTHLA |
| 47 | [⁶⁴Cu]LQVTDSGLYRCVIYHPPPYLDDFQKKWQEEM(O)ELY |
| | RQKVE |
| 48 | LQEEDAGEYGCMGEEM(O)RDRARAHV |
| 49 | LQEEDAGEYGCMWQEEM(O)ELYRQKV |
| 50 | IIVTDVIATLPLGEEMRDRARAHVDALRTHLA |
| 51 | IIVTDVIATLPYLDDFQKKWQEEMELYRQKVE |
| 52 | [⁶⁴Cu]IIVTDVIATLPLGEEMRDRARAHVDALRTHLA |
| 53 | [⁶⁴Cu]IIVTDVIATLPYLDDFQKKWQEEMELYRQKVE |
| 54 | IIVTDVIATLPLGEEM(O)RDRARAHVDALRTHLA |
| 55 | IIVTDVIATLPYLDDFQKKWQEEM(O)ELYRQKVE |
| 56 | [⁶⁴Cu]IIVTDVIATLPLGEEM(O)RDRARAHVDALRTHLA |
| 57 | [⁶⁴Cu]IIVTDVIATLPYLDDFQKKWQEEM(O)ELYRQKVE |
| 58 | PtxGFLSKSLVFPLGEEMRDRARAHVDALRTHLA |
| 59 | PtxGFLSKSLVFPYLDDFQKKWQEEMELYRQKVE |
| 60 | PtxGFLSKSLVFPLGEEM(O)RDRARAHVDALRTHLA |
| 61 | PtxGFLSKSLVFPYLDDFQKKWQEEM(O)ELYRQKVE |
| 62 | IVILLAGGFLSKSLVFSVLFAPLGEEMRDRARAHVDALRTHL |
| | A |
| 63 | IVILLAGGFLSKSLVFSVLFAPYLDDFQKKWQEEMELYRQKV |
| | E |
| 64 | IVILLAGGFLSKSLVFSVLFAPLGEEM(O)RDRARAHVDALR |
| | THLA |
| 65 | IVILLAGGFLSKSLVFSVLFAPYLDDFQKKWQEEM(O)ELYR |
| | QKVE |

### Antitumor Efficacy of TREM-1 Inhibitory Treatment

Although currently, no data are available on the antitumor activity of type I TREM-1 inhibitors that include but are not limited to, blocking (antagonistic, inhibiting) anti-TREM-1 antibodies and/or their fragments and peptide inhibitors LP17 and LR12, in certain embodiments of the present inventions, these and other type I TREM-1 inhibitors can be used in monotherapy and combination-therapy treatment regimen with other cancer therapies to prevent and/or treat cancer.

### Monotherapy Treatment Regimen

In some embodiments, monotherapy treatment with 25 mg/kg free type II TREM-1 peptide inhibitor GF9 and 2.5 mg/kg GF9 incorporated into the carrier—dSLP (GF9-dSLP) or sSLP (GF9-sSLP), inhibits tumor growth in the human As-PC-1, Bx-PC-3 and Capan-1 xenograft mouse models of PC compared with vehicle-treated cancer mice (See FIG. 4). In one embodiment, the antitumor efficacy of GF9 treatment depends on the xenograft and formulation used (See FIG. 4). In one embodiment, the antitumor efficacy of 25 mg/kg GF9 is comparable with that of 2.5 mg/kg GF9-dSLP or 2.5 mg/kg GF9-sSLP (See FIG. 4). In one embodiment, the antitumor efficacy of GF9 treatment is comparable with that of 20 mg/kg paclitaxel (PTX). In one embodiment, the antitumor efficacy of 2.5 mg/kg GF9-sSLP in the BxPC-3 xenograft mouse model of PC is higher than that of 2.5 mg/kg GF9-dSLP (See FIG. 4).

In some embodiments, as measured by body weight changes, monotherapy long-term treatment of mice with human As-PC-1, Bx-PC-3 and Capan-1 xenografts with 25 mg/kg GF9, 2.5 mg/kg GF9-dSLP or 2.5 mg/kg GF9-sSLP does not induce any acute toxicity and is well tolerable (See FIG. 5).

In some embodiments, monotherapy treatment with 4 mg/kg of an equimolar mixture of GA31 (SEQ ID NO. 26) and GE31 (SEQ ID NO. 27) incorporated into the carrier—dSLP (GA/E31-dSLP) or sSLP (GA/E31-sSLP), inhibits tumor growth in the human As-PC-1, Bx-PC-3 and Capan-1 xenograft mouse models of PC compared with vehicle-treated cancer mice (See FIG. 6). In one embodiment, the antitumor efficacy of the treatment depends on the xenograft and formulation used (See FIG. 6). In one embodiment, the antitumor efficacy of 4 mg/kg GA/E31-dSLP and GA/E31-sSLP treatment is comparable with that of 20 mg/kg PTX.

In some embodiments, as measured by body weight changes, monotherapy long-term treatment of mice with human As-PC-1, Bx-PC-3 and Capan-1 xenografts with 4 mg/kg GA/E31-dSLP and GA/E31-sSLP, does not induce any acute toxicity and is well tolerable (See FIG. 7).

In some embodiments, monotherapy treatment with 25 mg/kg GF9, 2.5 mg/kg GF9-dSLP or 2.5 mg/kg GF9-sSLP prolongs survival of mice with human As-PC-1, Bx-PC-3 and Capan-1 xenografts compared with vehicle-treated cancer mice. In one embodiment, the efficacy of 25 mg/kg GF9, 2.5 mg/kg GF9-dSLP or 2.5 mg/kg GF9-sSLP in improving survival of cancer mice is comparable with that of 20 mg/kg PTX. In some embodiments, monotherapy treatment with 25 mg/kg GF9 is less effective in improving survival of mice with human AsPC-1 and Capan-1 xenografts compared with 2.5 mg/kg GF9-dSLP or 2.5 mg/kg GF9-sSLP (See FIG. 8). In one embodiment, 2.5 mg/kg GF9-dSLP is less effective in improving survival of mice with human BxPC-3 xenografts compared with 2.5 mg/kg GF9-sSLP (See FIG. 8).

In some embodiments, monotherapy treatment with 4 mg/kg GA/E31-dSLP or 4 mg/kg GA/E31-sSLP prolongs survival of mice with human As-PC-1, Bx-PC-3 and Capan-1 xenografts compared with vehicle-treated cancer mice. In one embodiment, the efficacy of 4 mg/kg GA/E31-dSLP or 4 mg/kg GA/E31-sSLP in improving survival of cancer mice is comparable with that of 20 mg/kg PTX.

In some embodiments, the antitumor effect of monotherapy treatment with 25 mg/kg GF9, 2.5 mg/kg GF9-dSLP, 2.5 mg/kg GF9-sSLP, 4 mg/kg GA/E31-dSLP or 4 mg/kg GA/E31-sSLP persists even after treatment has been completed (See FIGS. 4 and 6).

In certain embodiments, the observed antitumor effect of monotherapy treatment of cancer mice with GF9 is dose-dependent and specific: administration of GF9 at 2.5 mg/kg or a control peptide GF9-G (GFLSGSLVF) at 25 mg/kg does not affect tumor growth compared with vehicle-treated cancer mice.

In certain embodiments, the observed antitumor effect of monotherapy treatment of cancer mice with 25 mg/kg GF9, 2.5 mg/kg GF9-dSLP, 2.5 mg/kg GF9-sSLP, 4 mg/kg GA/E31-dSLP or 4 mg/kg GA/E31-sSLP correlates with the intratumoral macrophage content (as measured by immunostaining with F4/80 antibodies): the higher the intratumoral macrophage content, the higher is the antitumor activity of the tested SCHOOL TREM-1 inhibitory GF9 sequences (See FIG. 10), suggesting that the measured intratumoral macrophage content may predict response to TREM-1 inhibitory treatment.

In one embodiment, monotherapy treatment of mice with human BX-PC3 xenografts with 25 mg/kg GF9, 2.5 mg/kg GF9-sSLP or 4 mg/kg GA/E31-sSLP significantly suppresses intratumoral macrophage infiltration (as measured by immunostaining with F4/80 antibodies) compared with vehicle-treated cancer mice.

In certain embodiments, monotherapy treatment of mice with human AsPC-1, BX-PC3 and Capan-1 xenografts with 25 mg/kg GF9 or 2.5 mg/kg GF9-sSLP results in reduction of serum levels of IL-11D, IL-6 and CSF-1 compared with vehicle-treated cancer mice (See FIG. 12).

In one embodiment, monotherapy treatment of mice with oxygen-induced retinopathy (OIR) with 25 mg/kg GF9, 2.5 mg/kg GF9-sSLP or 4 mg/kg GA/E31-sSLP results in reduction of tissue expression levels of TREM-1 and CSF-1 compared with vehicle-treated OIR mice (See FIG. 13) and prevents retinal neovascularization as described in (Shen and Sigalov 2017, Rojas et al. 2018). Although it is not necessary to understand the mechanism of an invention, it is believed that the observed reduction in serum and tissue expression level of CSF-1 (See FIGS. 12 and 13) can contribute to the anticancer and antiangiogenic activity of SCHOOL TREM-1 inhibitory GF9 sequences described in (Shen and Sigalov 2017, Rojas et al. 2018) similarly to the anticancer and antiangiogenic activity of CSF-1 inhibitors described in (Kubota et al. 2009). Importantly, these findings demonstrate that sSLP can pass the blood-retinal barrier (BRB) and deliver the incorporated agent (in this case, TREM-1 type II peptide inhibitors GF9, GA31 and GE31) to the target cells in the retina.

### Exemplary Combination-Therapy Treatment Regimen.

From the DRAFT PCT. As described in (Boussios et al. 2012), the toxicity of cancer chemotherapy is among the most important factors limiting its use. The toxicity of cancer chemotherapy is among the most important factors limiting its use. Gastrointestinal toxicity during chemotherapy is frequent and contributes to dose reductions, delays and cessation of cancer treatment. The development of intervention strategies that could eliminate an expected side effect of chemotherapy is vital. Developing new chemotherapy regimens with similar efficacy but less toxicity should be a priority for future research.

Another example is the lack of efficacy of immune checkpoint blockade (ICB) therapy (immunotherapy) in many types of cancer. For example, for patients with early liver cancer, surgery is the optimal treatment, but most patients are diagnosed with advanced liver cancer and thus miss the opportunity for surgery. Due to the limitations of targeted chemotherapeutic drugs Sorafenib and Regorafenib, the immune “brake-point” drug represented by the anti-PD-1/PD-L1 axis holds promise for curing patients with advanced liver cancer. At present, Opdivo (Nivolumab), a drug for the PD-L1/PD-1 axis approved by the FDA, achieved good results in various tumor treatments. However, liver cancer is characterized as rich in immunosuppressive cell infiltration, and the anti-PD-1/PD-L1 treatment is not effective. Preclinical studies reveal that the response rate of the anti-PD-1/PD-L1 treatment adds up to little more than 14.3%. Therefore, it is a top priority to reverse this situation, explore new strategies for fighting liver cancer, and improve prognosis in patients.

To overcome challenges associated with standalone chemo- and immunotherapy treatment regimens, combination-therapy treatments for cancer have become more common, in part due to the perceived advantage of attacking the disease via multiple avenues. Although many effective combination-therapy treatments have been identified over the past few decades; in view of the continuing high number of deaths each year resulting from cancer, a continuing need exists to identify effective therapeutic regimens for use in anticancer treatment.

As described herein, surprisingly it was found that TREM-1 inhibitory treatment synergizes with chemo- and immunotherapies in suppressing tumor growth and improving survival rates in cancer mice. While not being bound to any particular theory, it is believed that this synergism is mediated by affecting tumor microenvironment and reducing the immunosuppressive activity of myeloid-derived suppressor cells through modulation of the TREM-1/DAP-12 signaling pathway.

In some embodiments, in subcutaneous EMT-6 syngeneic breast tumor model in BALB/C mice, a valuable pre-clinical model for immuno-oncology studies of triple negative breast cancer (TNBC), intraperitoneal treatment of cancer mice for 7 days with 2.5 mg/kg GF9-sSLP combined with immunotherapy treatment (anti-PD-L1 antibody) synergistically inhibits tumor growth and improves survival. In one embodiment, the late surviving mice who received combination therapy, have much more energy and no pain behaviors as compared to other animals, suggesting the lack of acute toxicity of this combination-therapy treatment regimen.

In certain embodiments, in subcutaneous CT26 syngeneic colon tumor mouse model in BALB/C mice, a valuable pre-clinical model for immuno-oncology studies of colorectal cancer, intraperitoneal treatment of cancer mice for 7 days with 2.5 mg/kg GF9-sSLP combined with immunotherapy treatment (anti-PD1 antibody) synergistically inhibits tumor growth.

In certain embodiments, in C57BL/6 mice intracranially implanted with GL261 glioblastoma tumor cells into the right caudate putamen, a valuable pre-clinical model for studies of glioblastoma multiforme (GBM), intraperitoneal treatment of cancer mice administered radiation therapy (RT; 2 Gy of whole brain radiation, one-field posterior anterior, every other day to a total dose of 6 Gy) with 2.5 mg/kg GF9-sSLP for 10 days synergistically improves survival of mice as compared to standalone RT or GF9-sSLP therapies, which both do not improve survival as compared to the vehicle-treated, not irradiated mice. In one embodiment, quantitative polymerase chain reaction (qPCR) analysis shows a 6-fold decrease of mRNA levels of TGF-β, previously noted to be regulated by TREM-1 signaling in vitro, in TAMs in GF9-sSLP-treated mice as compared to controls. Importantly, these findings demonstrate that sSLP can pass the BBB and deliver the incorporated agent (in this case, TREM-1 type II peptide inhibitor GF9) to the target cells in the brain. While not being bound to any particular theory, it is believed that the ability of these particles to penetrate the BBB is mediated by the putative scavenger receptor BI (SRBI)-binding epitope on one of their apo A-I peptide constituent.

As described in (Wu et al. 2019), in orthotopic Hepa 1-6 syngeneic hepatocellular carcinoma model in C57BL/6 mice, a valuable pre-clinical model for immuno-oncology studies of liver cancer, intraperitoneal daily treatment of cancer mice with 25 mg/kg murine GF9 peptide (mGF9, GLLSKSLVF) combined with immunotherapy treatment (anti-PD-L1 antibody) synergistically inhibits tumor growth and improves survival as compared to standalone anti-PD-L1 or GF9 therapies. It should be noted, that while standalone 25 mg/kg GF9 treatment inhibits tumor growth in this model, standalone anti-PD-L1 therapy or treatment with a control murine GF9-G peptide (mGF9-G, GLLSGSLVF) do not affect tumor growth and survival of mice.

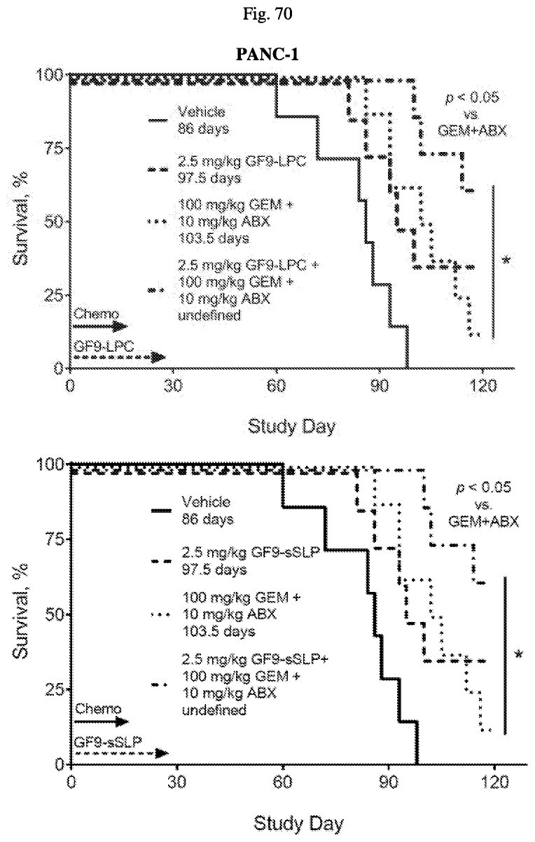

In certain embodiments, in the human PANC-1 xenograft model in athymic nude mice for human pancreas carcinoma, a valuable pre-clinical model for studies of pancreatic cancer, intraperitoneal treatment of cancer mice with 2.5 mg/kg GF9-sSLP (once daily 5 times per week) for 28 days combined with a standard chemotherapy treatment regimen with 100 mg/kg gemcitabine (GEM)+10 mg/kg abraxane (ABX) (days 1, 4, 8, 11, 15) synergistically inhibits tumor growth (See FIG. 14). In one embodiment, in this model, standalone GF9-sSLP therapy is as effective in suppressing tumor growth as a standalone GEM+ABX standard chemotherapy (See FIG. 15). In one embodiment, when combined with a standard GEM+ABX therapy, GF9-sSLP treatment exhibits a synergistic effect in inhibiting tumor growth a month later after the treatment has been completed and this effect persists till the final measurement endpoint (See FIG. 14). In one embodiment, in this model, intraperitoneal standalone treatment of cancer mice with 2.5 mg/kg GF9-sSLP (once daily 5 times per week) for 28 days is as effective in inhibiting tumor growth as a standalone standard chemotherapy treatment regimen with 100 mg/kg GEM+10 mg/kg ABX (days 1, 4, 8, 11, 15) (See FIG. 70).

In certain embodiments, as measured by body weight changes, intraperitoneal treatment with 2.5 mg/kg GF9-sSLP (once daily 5 times per week) for 28 days standalone or combined with a standard chemotherapy treatment regimen with 100 mg/kg GEM+10 mg/kg ABX (days 1, 4, 8, 11, 15) is well tolerable in athymic nude mice bearing the human PANC-1 xenografts and shows no acute toxicity in contrast to a standalone GEM+ABX chemotherapy treatment (See FIG. 70).

In certain embodiments, in the human PANC-1 xenograft model in athymic nude mice for human pancreas carcinoma, intraperitoneal treatment of cancer mice with 2.5 mg/kg GF9-sSLP (once daily 5 times per week) for 28 days combined with a standard chemotherapy treatment regimen with 100 mg/kg GEM+10 mg/kg ABX (days 1, 4, 8, 11, 15) synergistically improves survival 3-fold as compared to standalone GF9-sSLP treatment and a standalone GEM+ABX chemotherapy treatment (See FIG. 70).

In one embodiment, as measured by body weight changes, human GF9 peptide (GFLSKSLVF) is well tolerable and shows no acute toxicity in healthy C57BL/6 mice up to at least 300 mg/kg dose (See FIG. 70).

The synergistic antitumor activity of TREM-1 inhibitory therapy using GF9, GF9-SLP and GA/E31-SLP when combined with chemotherapy, immunotherapy or RT in different types of cancer described herein suggest a potential use of TREM-1 inhibitory therapy as an induction therapy for the treatment of cancer in combination with cancer therapies including but not limited to, anticancer vaccine, an anticancer immunotherapy agent, anti-cancer immunomodulatory agent, an additional anticancer therapeutic, RT, surgery or a combination thereof.

The data on safety, well tolerability and lack of acute toxicity of TREM-1 inhibitory therapy with GF9, GF9-SLP and GA/E31-SLP formulations in animal models of various cancer types described herein, as well as those disclosed in U.S. Pat. Nos. 8,513,185 and 9,981,004 and described in (Sigalov 2014, Shen and Sigalov 2017) are in line with the data reported for TREM-1 inhibitory therapy using LR12 peptide in healthy and septic human subjects as described in (Cuvier et al. 2018, Francois et al. 2018), suggesting a safety of TREM-1 inhibitory therapy strategy in humans.

Considering that as described herein, TREM-1 inhibitory therapy using well tolerable treatment of cancer mice with GF9-sSLP exhibits a synergistic effect in inhibiting tumor growth a month later after the treatment has been completed and this effect persists till the final measurement endpoint, this therapy could be a good candidate for its use as a safe and well tolerable standalone maintenance therapy after an induction therapy using any other anticancer therapies and combination-therapy treatment regimen or their combinations with TREM-1 inhibitory therapy. TREM-1 therapy could also used as a second-line treatment as a standalone therapy or in combination with other anticancer therapies if cancer recurs or progresses after the prior therapy.

In certain embodiments, the modulators and compositions described herein can be used in combination with other therapeutic antitumor agents including but not limiting to, to those described in (Page et al. 2009) and disclosed in U.S. Pat. Nos. 4,427,660; 9,161,988; 8,921,314; 8,022,047; 8,680,139; 9,717,717; US 20150005355; US 20140275026; US 20150306085; U.S. Pat. Nos. 9,320,811 and 9,173,891 (see also TABLE 3).

In certain embodiments, the modulators and compositions described herein may be administered for the treatment of a cancer in a combination therapy with other suitable treatment modalities that may include, without limitation, administration of radiation therapy, e.g., gamma radiation therapy. Other suitable treatment modalities may include, for example, administering to a patient in combination with an anticancer vaccine, an anticancer immunotherapy agent, anticancer immunomodulatory agent, an additional anticancer therapeutic, or a combination thereof.

Anticancer vaccines may include, for example, Gardasil and Cervarix (prophylactic) and Sipuleucel-T/Provenge (therapeutic). Anticancer immunotherapy agents may include, for example, Alemtuzumab, Ipilimumab, Nivolumab, Pembrolizumab, Rituximab, Nivolumab, Interferon, and Interleukin. Anticancer immunomodulatory agents may include for example, thalidomide, lenalidommide, and pomalimomide. Additional anticancer therapeutics may include, for example, an alkylating agent, a tubulin inhibitor, a proteasome inhibitor, a topoisomerase inhibitor (I and II), a CHK1 inhibitor, a CHK2 inhibitor, a PARP inhibitor, doxorubicin, epirubicin, vinblastine, etopside, topotecan, bleomycin, temozolomide, gemcitabine, paclitaxel, a nanoparticle albumin-bound paclitaxel (Abraxane), and mytomycin c. Alkylating agents may be selected from the group consisting of Dacarbazine, Procarbazine, Carmustine, Lomustine, Uramustine, Busulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin. Tubulin inhibitors may be selected from the group consisting of Taxol, Docetaxel, Vinblastin, Epothilone, Colchicine, Cryptophycin, BMS-347550, Rhizoxin, Ecteinascidin, Dolastin 10, Cryptophycin 52, and IDN-5109. Proteasome inhibitors may be selected from the group consisting of Velcade (bortezomib), and Kyprolis (carfilzomib). Topoisomerase I inhibitors may be selected from the group consisting of Irinotecan, Topotecan, and Camptothecins (CPT). Topoisomerase II inhibitors may be selected from the group consisting of Amsacrine, Etoposide, Teniposide, Epipodophyllotoxins, and ellipticine. CHK1 inhibitors may be selected from the group consisting of TCS2312, PF-0047736, AZ07762, A-69002, and A-641397. PARP inhibitors may be selected from the group consisting of Olaparib, ABT-888, (veliparib), KU-59436, AZD-2281, AG-014699, BSI-201, BGP-15, INO-1001, ONO-2231 and the like.

Through combination therapy, reduction of adverse drug reaction and potentiation of anticancer activity are achievable by the combined effects of anticancer agents having different mechanisms of action, including reduction of the non-sensitive cell population; prevention or delaying of occurrence of drug resistance; and dispersing of toxicity by means of a combination of drugs having different toxicities.

When an anticancer agent used in combination has a particular medication cycle, it is preferable to establish an appropriate medication cycle for the modulators and compositions described herein, and such anticancer agent, so that the desired effects are attained. Specifically, the frequency of administration, dosage, time of infusion, medication cycle, and the like, may be determined properly according to individual cases, considering the kind of anticancer agent, state of the patients, age, gender, etc.

In using the combination therapy of the present invention, the same dose as that usually given as a monotherapy or a slightly reduced dose (for example, 0.10-0.99 times the highest dose as a single agent) may be given through a normal administration route.

The methods of the present invention will normally include medical follow-up to determine the therapeutic or prophylactic effect brought about in the patient undergoing treatment with the compound(s) and/or composition(s) described herein. Efficacy of the methods may be assessed on the basis of tumor regression, e.g., reducing the size and/or number of neoplasms, inhibition of tumor metastasis, decrease in a serological marker of disease, or other indicator of an inhibitory or remedial effect.

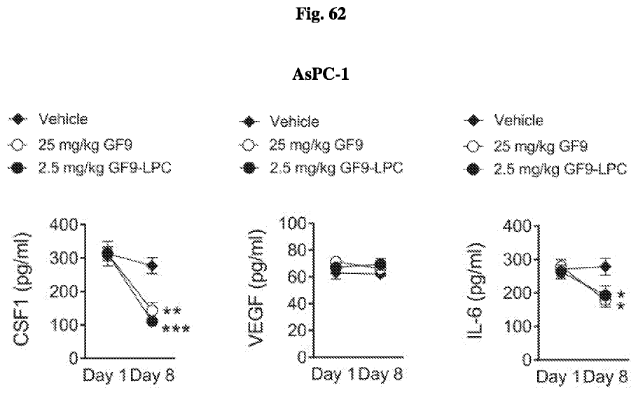

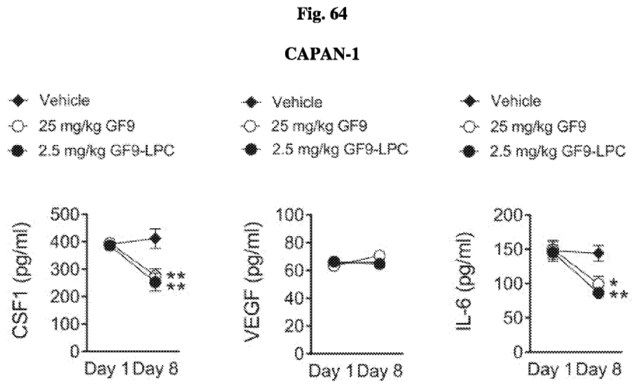

In one embodiment, FIG. 62 shows that inhibition of TREM-1/DAP-12 signaling using TREM-1 inhibitory peptide GF9 (GFLSKSLVF) in free form and bound to macrophage-specific lipopeptide complexes (GF9-LPC) reduces serum levels of CSF1 in human pancreatic cancer AsPC-1-bearing mice (see Shen and Sigalov. Mol Pharm 2017, 14:4572-4582). In one embodiment, FIG. 63 shows that inhibition of TREM-1/DAP-12 signaling using GF9 and LPC reduces serum levels of CSF1 in human pancreatic cancer BxPC-3-bearing mice (see Shen and Sigalov. Mol Pharm 2017, 14:4572-4582). In one embodiment, FIG. 64 shows that inhibition of TREM-1/DAP-12 signaling using GF9 and GF9-LPC reduces serum levels of CSF1 in human pancreatic cancer CAPAN-1-bearing mice (see Shen and Sigalov. Mol Pharm 2017, 14:4572-4582).

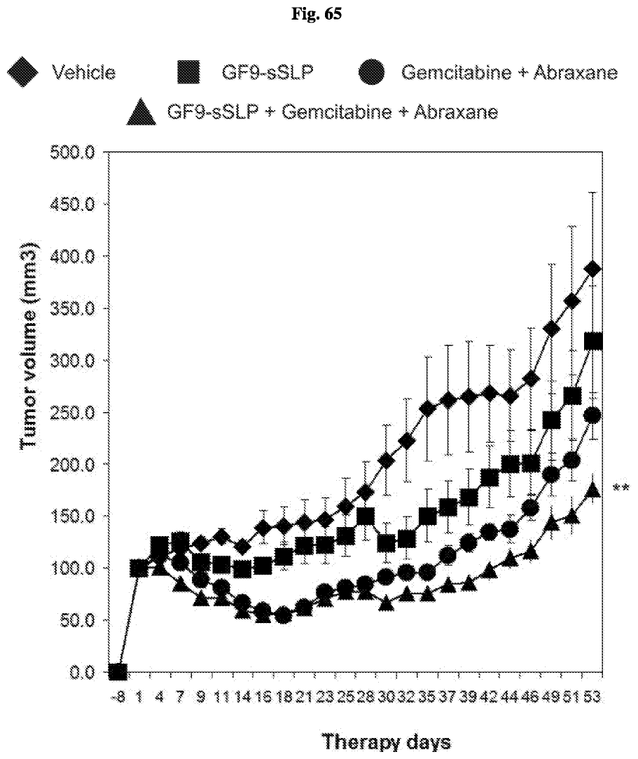

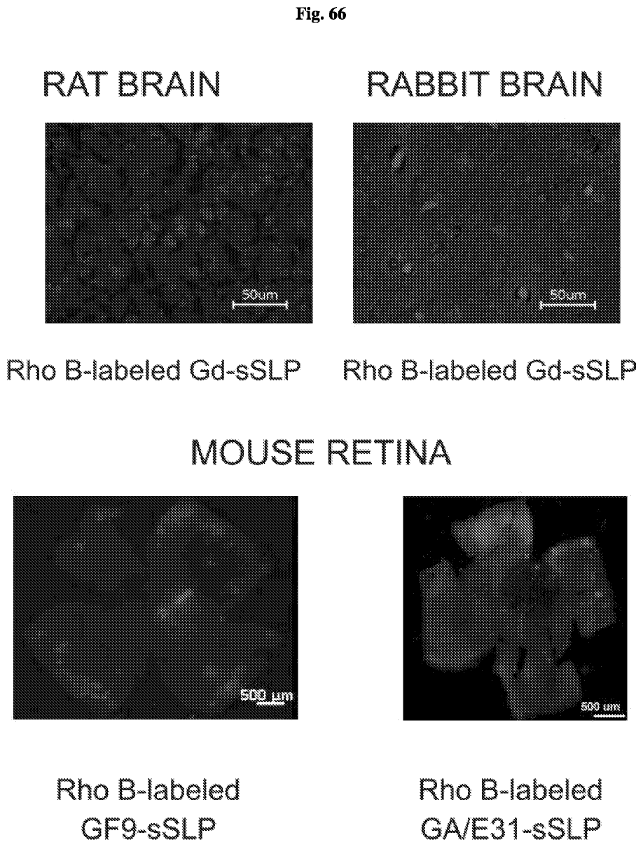

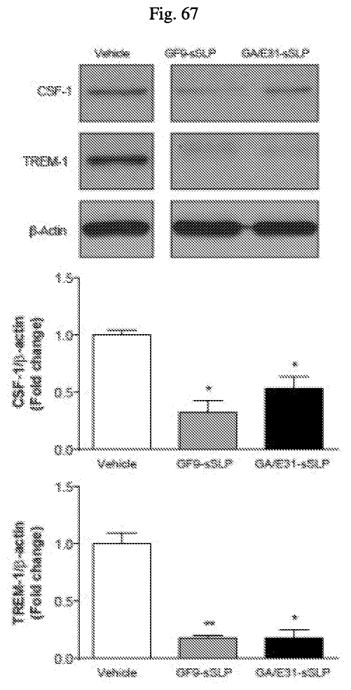

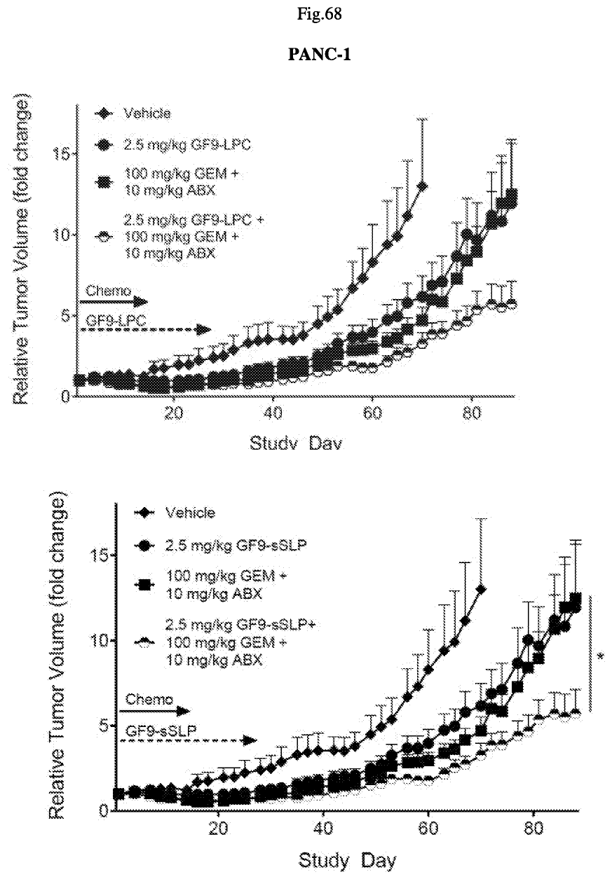

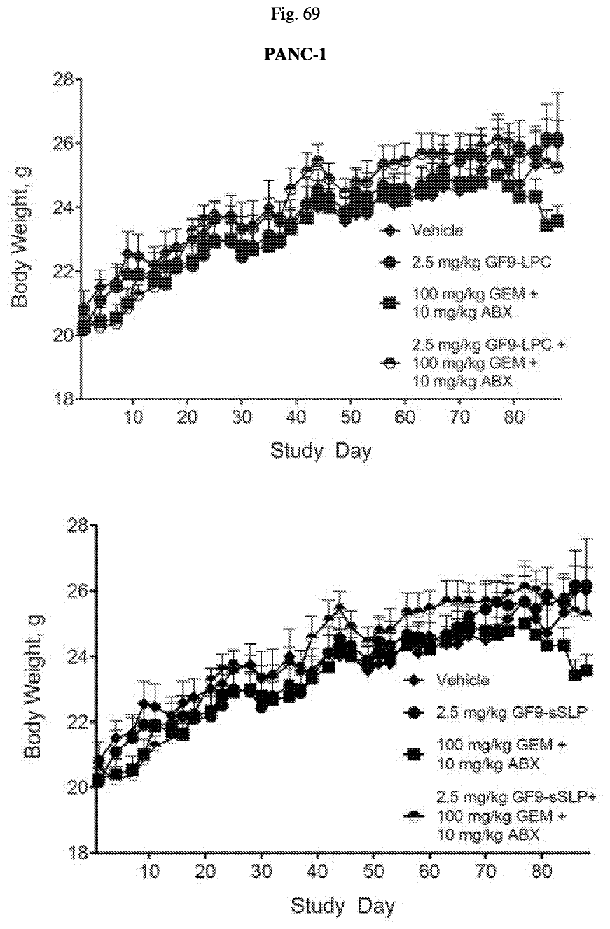

In some embodiments, FIG. 65 demonstrates that treatment with GF9, GF9-LPC or LPC comprising lipids and an equimolar mixture of the 31 amino acids-long TREM-1 modulatory peptide GA31 (GFLSKSLVFPLGEEM(O)RDRARAHVDALRTHLA) where M(O) is a methionine sulfoxide residue and the 31 amino acids-long TREM-1 modulatory peptide GE31 (GFLSKSLVFPYLDDFQKKWQEEM(O)ELYRQKVE) where M(O) is a methionine sulfoxide residue (GA/E31-LPC) inhibits tumor growth in mice bearing AsPC-1, BxPC-3 and CAPAN-1 human pancreatic cancers (see Shen and Sigalov. Mol Pharm 2017, 14:4572-4582). In some embodiments, FIG. 68 demonstrates that treatment with GF9, GF9-LPC or GA/E31-LPC is well tolerable in mice bearing AsPC-1, BxPC-3 and CAPAN-1 human pancreatic cancers (see Shen and Sigalov. Mol Pharm 2017, 14:4572-4582).

In some embodiments, FIG. 68 demonstrates that treatment with GF9, GF9-LPC or GA/E31-LPC inhibits tumor growth in mice bearing A549 human non-small cell lung cancer (NSCLC) as effectively as 20 mg/kg paclitaxel (PTX) and is well tolerable in these mice (see Shen and Sigalov. Mol Pharm 2017, 14:4572-4582).

In one embodiment, FIG. 70 demonstrates that treatment with GF9, GF9-LPC or GA/E31-LPC reduces the macrophage content in the tumor in mice bearing BxPC-3 human pancreatic cancer (see Shen and Sigalov. Mol Pharm 2017, 14:4572-4582).

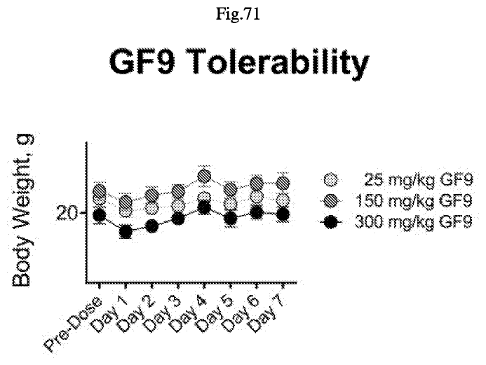

In one embodiment, FIG. 71 demonstrates that treatment with 2.5 mg/kg GF9-LPC inhibits tumor growth in mice bearing PANC-1 human pancreatic cancer as effectively as a combination of 100 mg gemcitabine (GEM) and 10 mg/kg nanoparticle albumin-bound PTX (nab-PTX, abraxane, ABX). In one embodiment, FIG. 71 further demonstrates that addition of 2.5 mg/kg GF9-LPC to a combination of 100 mg GEM and 10 mg/kg ABX sensitizes the tumor to the treatment with the 100 mg GEM and 10 mg/kg ABX chemotherapy in mice bearing PANC-1 human pancreatic cancer. In one embodiment, FIG. 71 further demonstrates that the synergistic effect of 2.5 mg/kg GF9-LPC with the 100 mg GEM and 10 mg/kg ABX chemotherapy in mice bearing PANC-1 human pancreatic cancer becomes more pronounced after completion of the treatment with GF9-LPC.

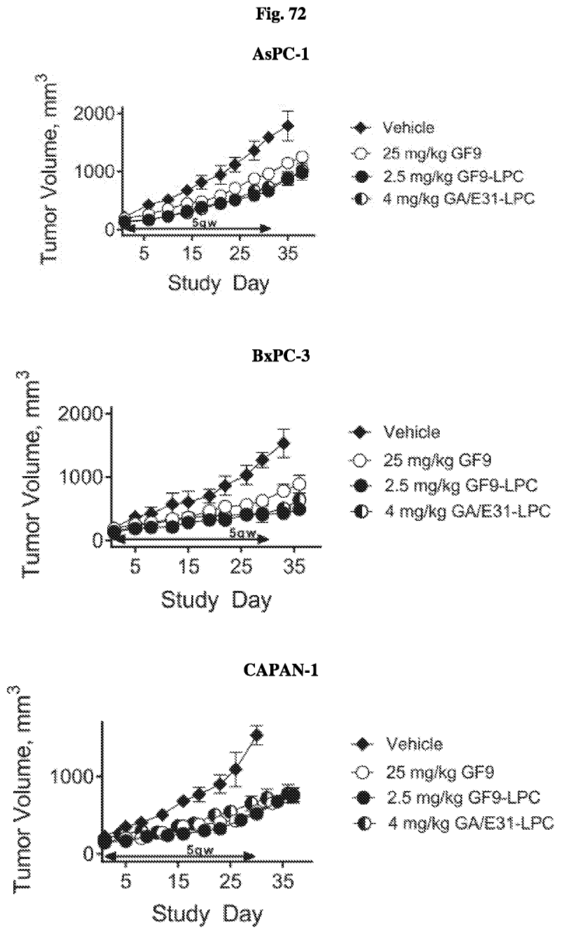

In one embodiment, FIG. 72 demonstrates that treatment with 2.5 mg/kg GF9-LPC is well tolerable in mice bearing PANC-1 human pancreatic cancer. In one embodiment, FIG. 72 FIG. 9 further shows that addition of 2.5 mg/kg GF9-LPC to a combination of 100 mg GEM and 10 mg/kg ABX does not worse tolerability of the combined treatment in mice bearing PANC-1 human pancreatic cancer.

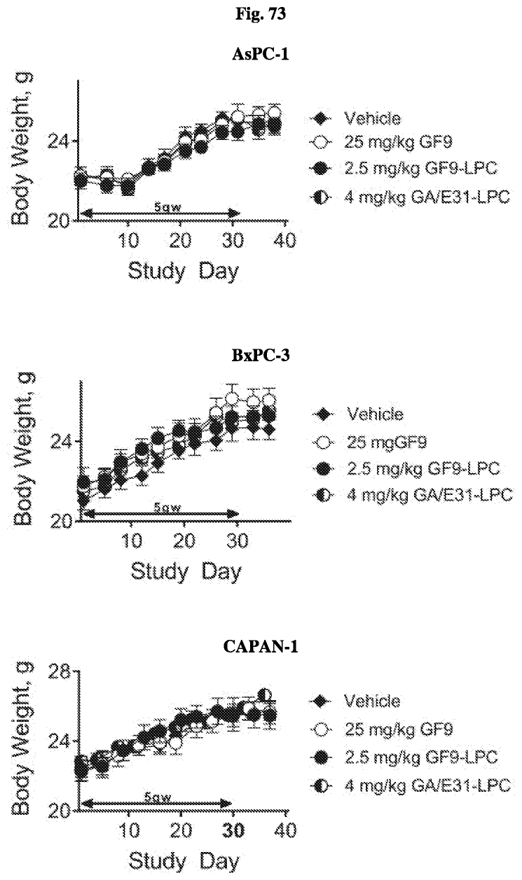

In one embodiment, FIG. 73 demonstrates that addition of 2.5 mg/kg GF9-LPC to a combination of 100 mg GEM and 10 mg/kg ABX significantly extends survival rate of mice bearing PANC-1 human pancreatic cancer as compared to the those treated with the 100 mg GEM and 10 mg/kg ABX chemotherapy alone.

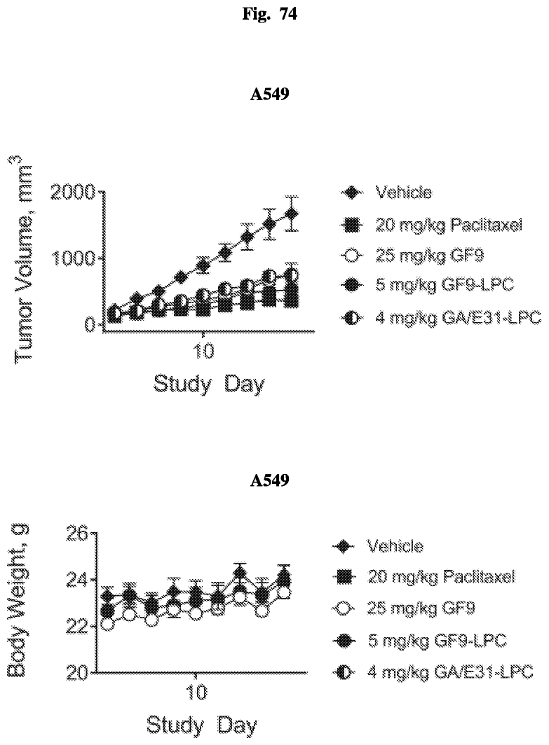

In one embodiment, FIG. 74 FIG. 11 demonstrates that treatment with 25 mg/kg GF9 is well tolerable in healthy mice up to at least 300 mg/kg (Sigalov 2014).

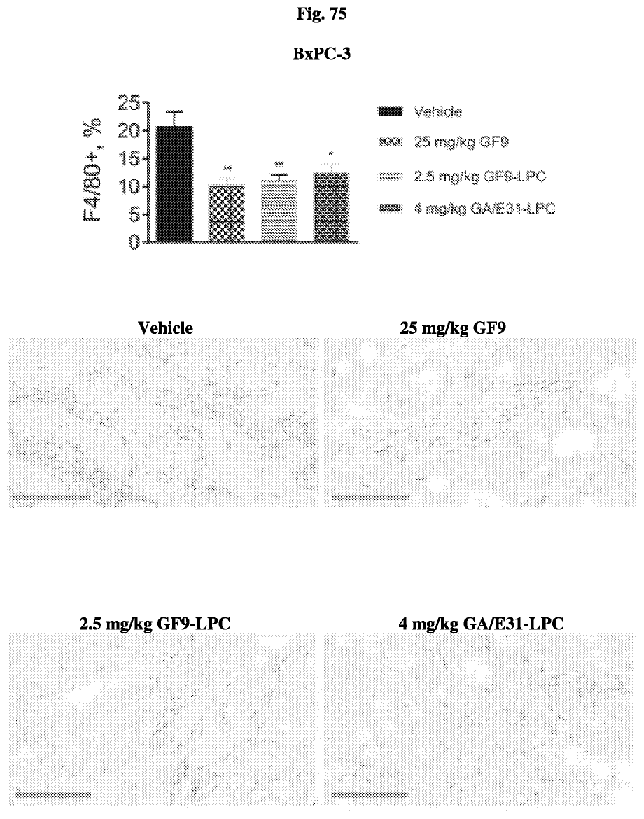

In one embodiment, FIG. 75 demonstrates that GF9, GF9-LPC and GA/E31-LPC penetrate the synovial membrane and ameliorate arthritis in mice with collagen-induced arthritis (CIA) (Shen and Sigalov 2017).

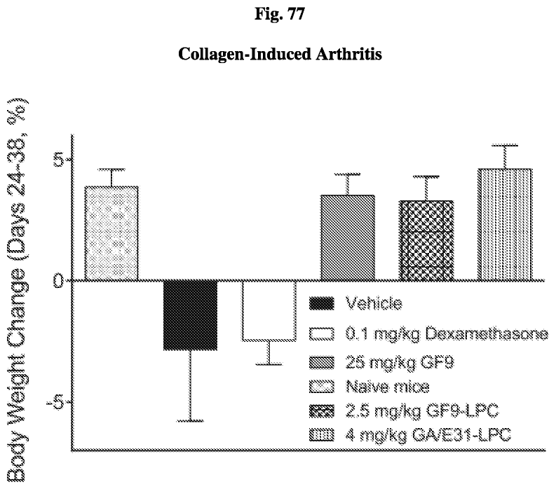

In one embodiment, FIG. 75 and FIG. 77 demonstrate that treatment with GF9, GF9-LPC and GA/E31-LPC reduces synovial inflammation and protects against bone and cartilage damage in mice with CIA (Shen and Sigalov 2017).

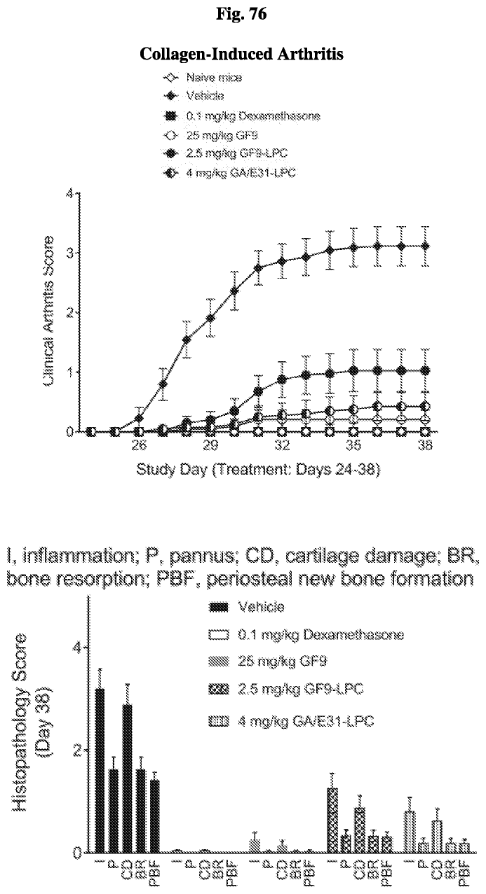

In one embodiment, FIG. 76 demonstrates that treatment with GF9, GF9-LPC and GA/E31-LPC is well tolerable in mice with CIA (Shen and Sigalov 2017).

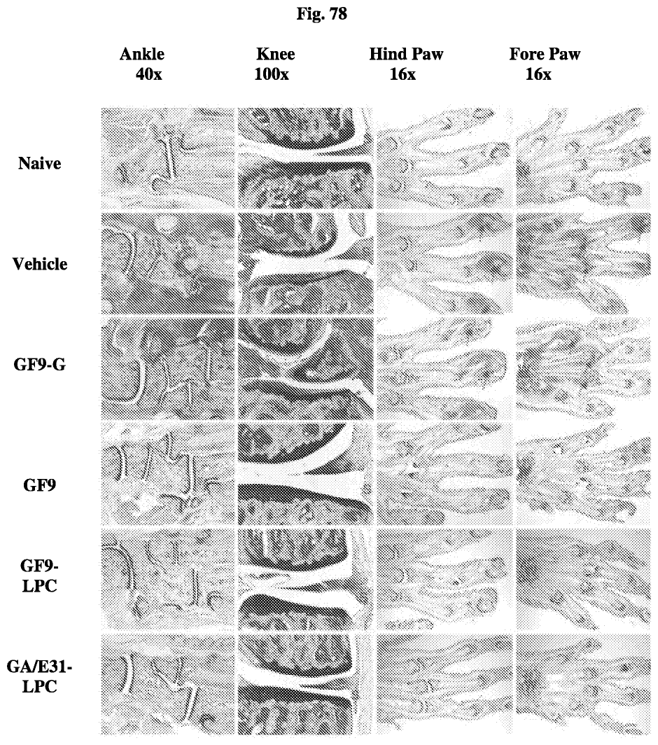

In some embodiments, FIG. 78 demonstrates that treatment with GF9, GF9-LPC and GA/E31-LPC reduces serum levels of CSF1 in mice with CIA (Shen and Sigalov 2017).

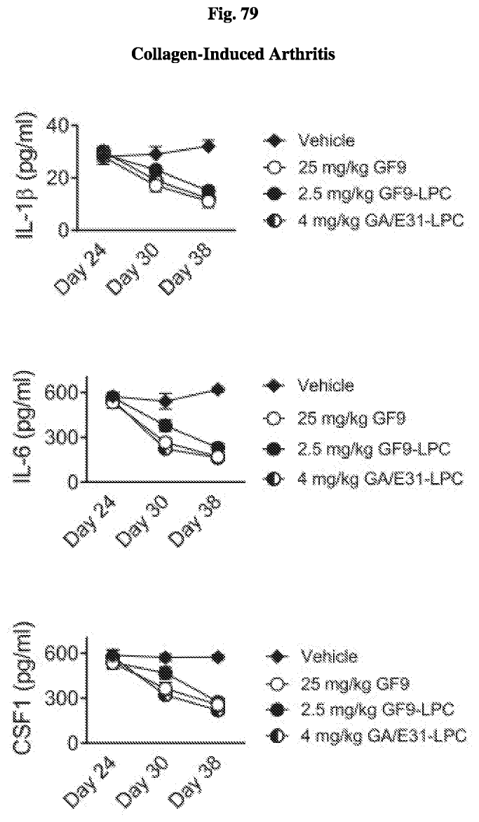

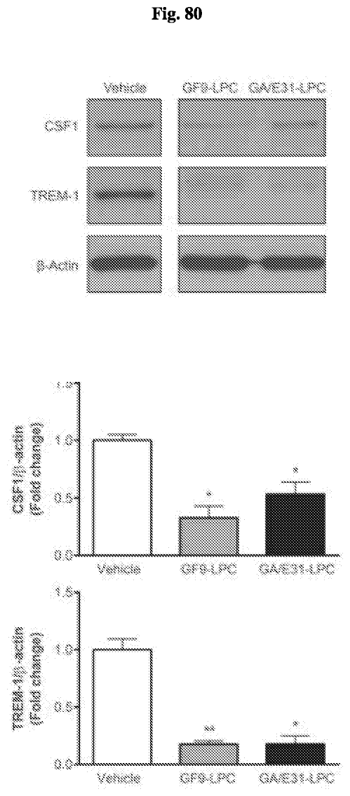

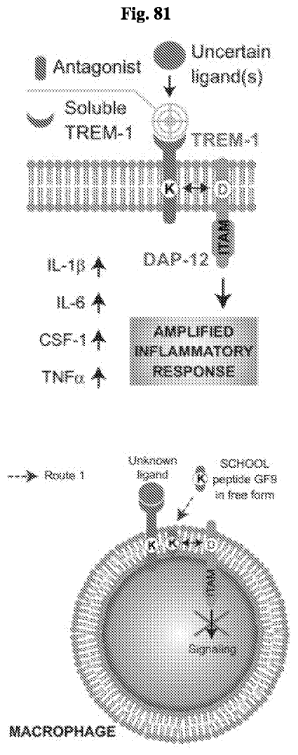

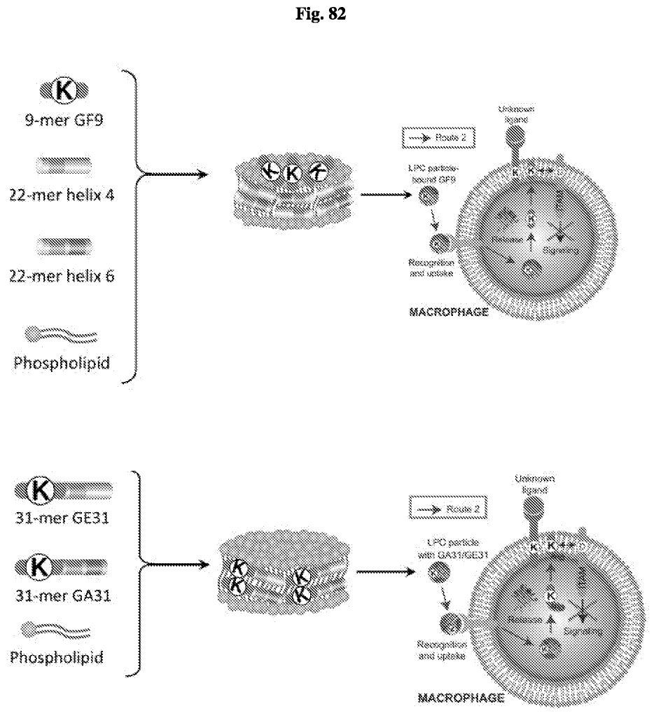

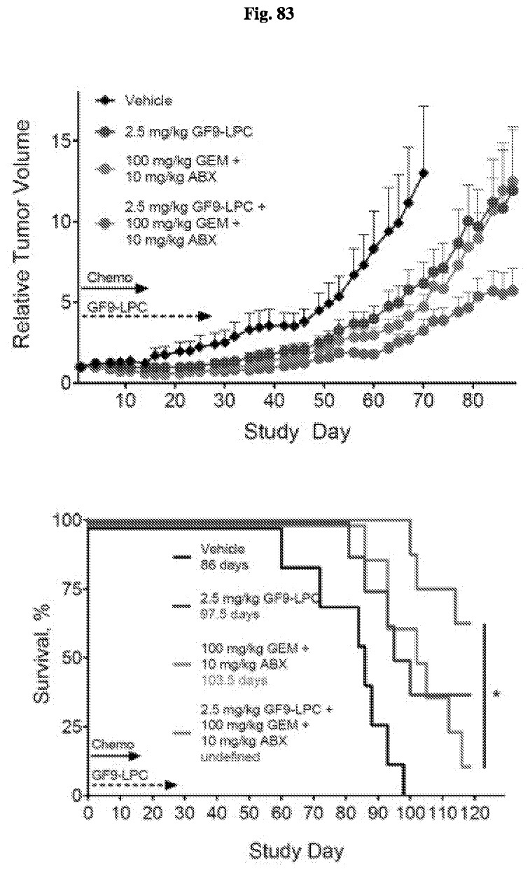

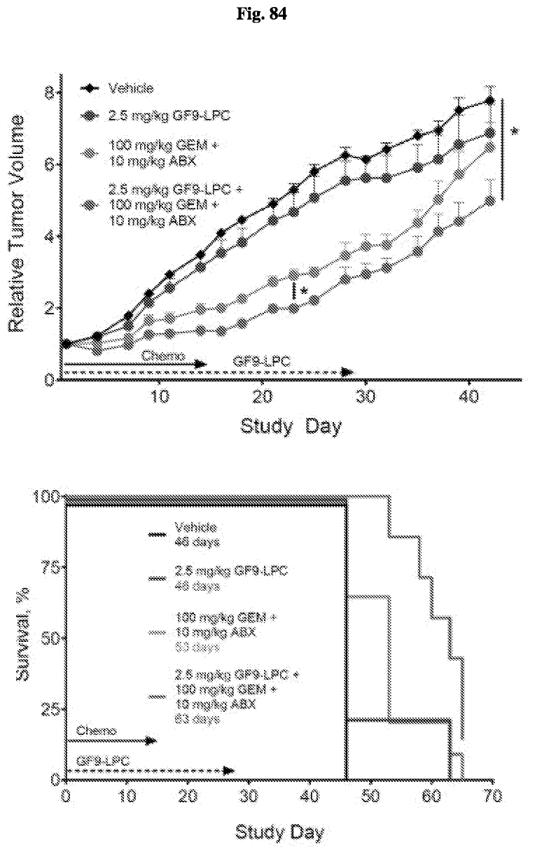

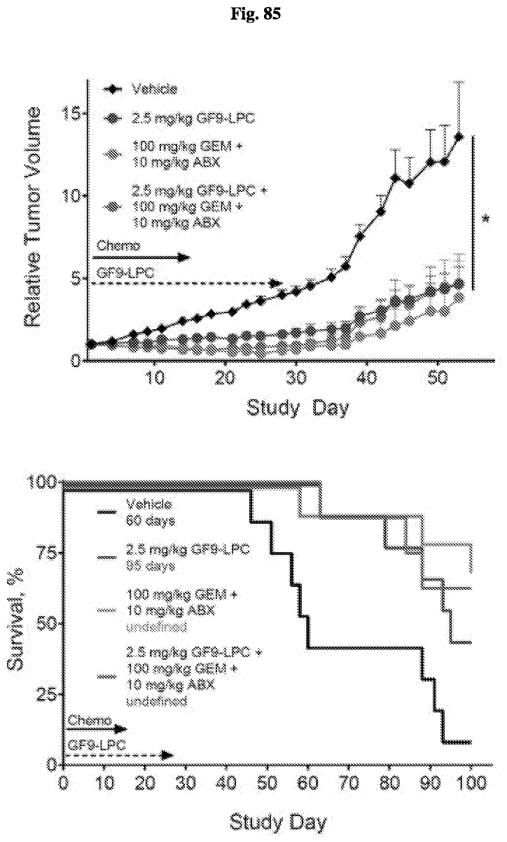

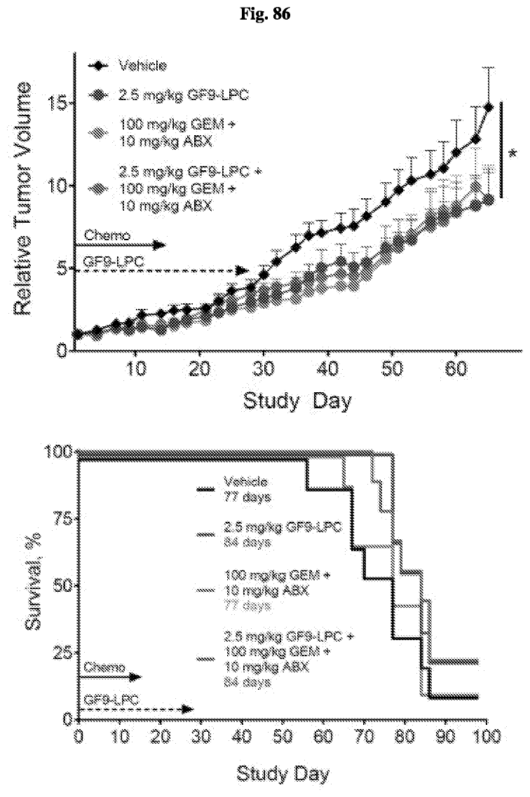

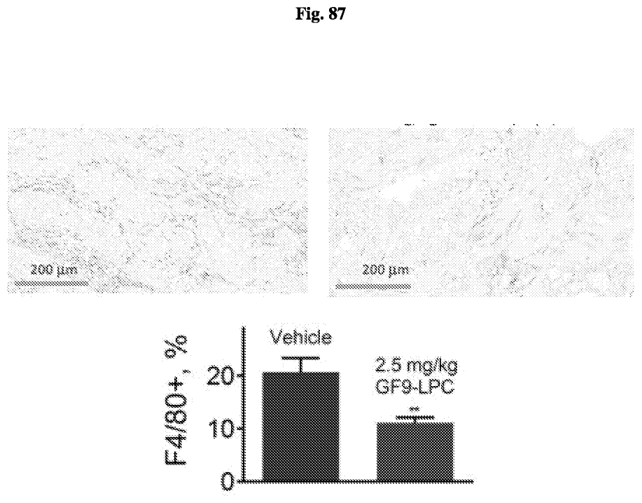

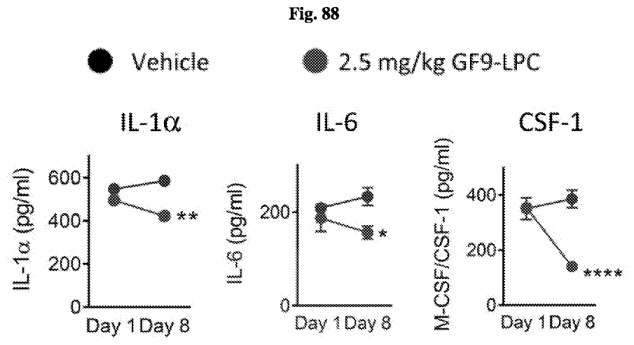

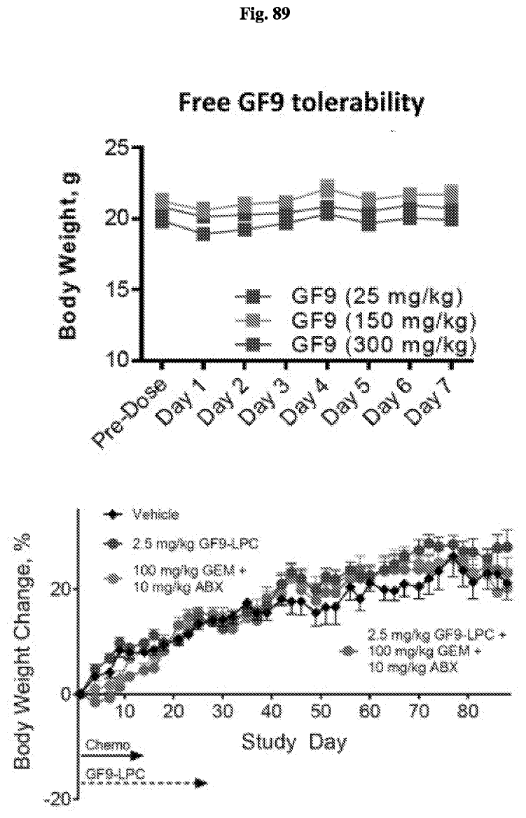

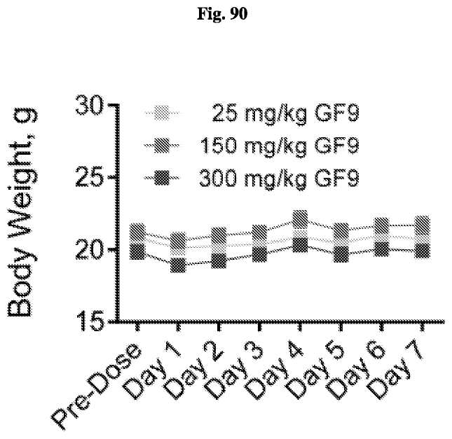

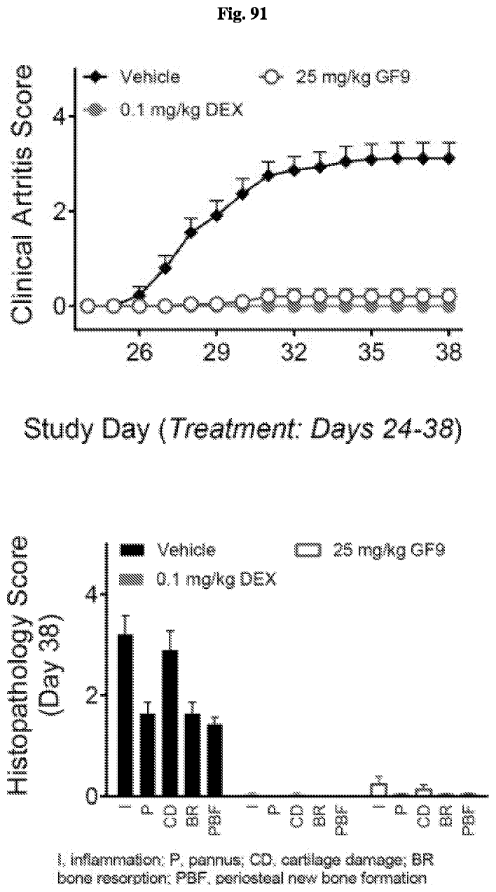

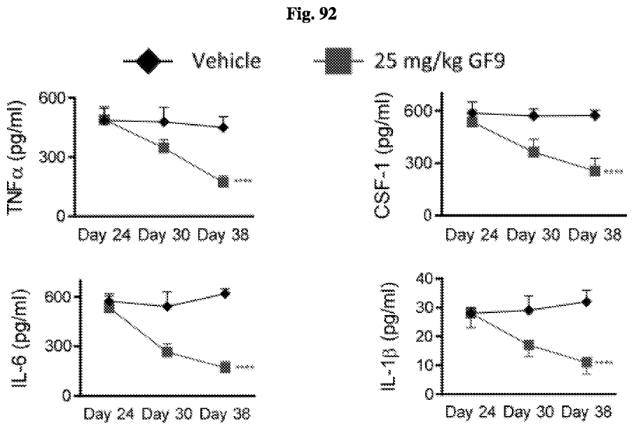

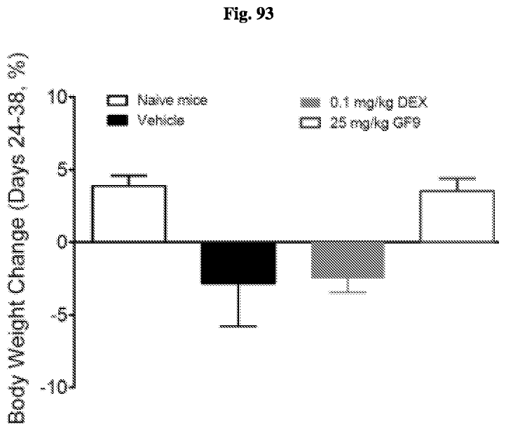

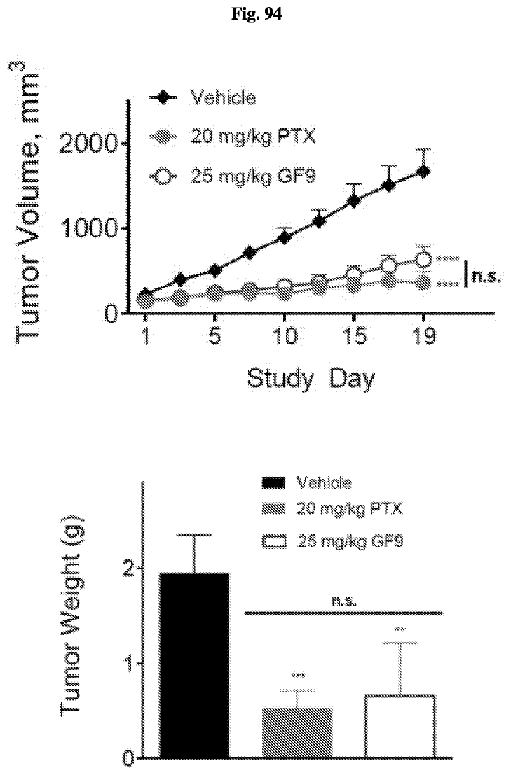

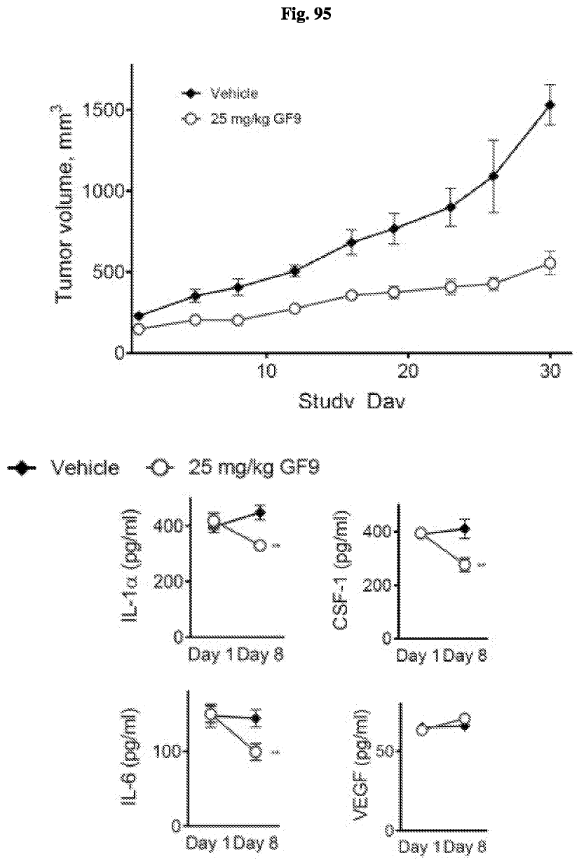

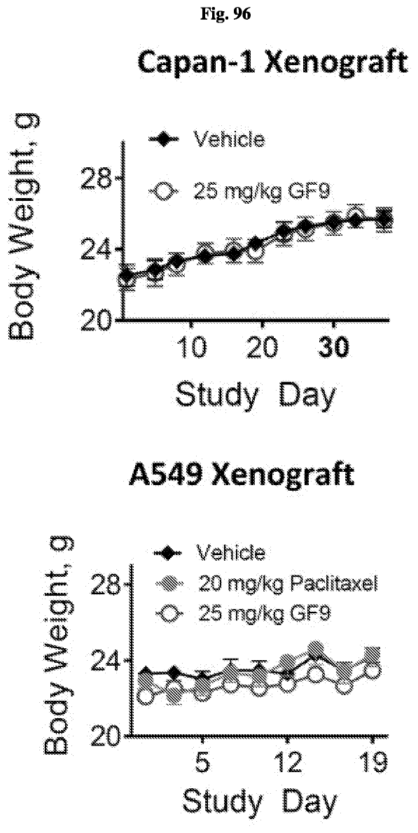

In some embodiments, FIG. 79 demonstrates that treatment with GF9-LPC and GA/E31-LPC reduces the levels of TREM-1 and CSF1 in the retina of mice with oxygen-induced retinopathy (OIR) (Rojas et al. 2018).

In certain embodiments, the preferred TREM-1 modulatory peptides and compositions of the invention can be used in combination with other TREM-1 inhibitory peptide sequences such as LR12 and LP17 (described in Gibot, et al. Infect Immun 2006, 74:2823-2830; Gibot, et al.

Shock 2009, 32:633-637; Gibot, et al. Eur J Immunol 2007, 37:456-466; Joffre, et al. J Am Coll Cardiol 2016, 68:2776-2793; Cuvier, et al. Br J Clin Pharmacol 2018, in press; Zhou, et al. Int Immunopharmacol 2013, 17:155-161; and disclosed in Faure, et al., U.S. Pat. No. 8,013,116; Faure, et al., U.S. Pat. No. 9,273,111; Gibot, et al., U.S. Pat. No. 9,657,081; Gibot and Derive, U.S. Pat. No. 9,815,883; and in Gibot and Derive, U.S. Pat. No. 9,255,136).

In certain embodiments, other therapeutic antitumor agents including but not limiting to, to those described in Page and Takimoto. Principles of chemotherapy. In: Pazdur R, Wagman L D, Camphausen K A, editors. Cancer Management: A Multidisciplinary Approach. 11th ed. Manhasset, N.Y.: Cmp United Business Media; 2009. p. 21-37; Sipsas, et al., Therapy of Mucormycosis, J Fungi (Basel) 2018, 4; Lin, et al. Chem Commun (Camb) 2013, 49:4968-4970; and in Turner, et al. Peptide Conjugates of Oligonucleotide Analogs and siRNA for Gene Expression Modulation. In: Langel U, ed, editor. Handbook of Cell-Penetrating Peptides. 2nd edition ed. Boca Raton: CRC Press; 2007. p. 313-328 and disclosed in Schiffman and Altman, U.S. Pat. No. 4,427,660; Castaigne, et al., U.S. Pat. No. 9,161,988; Castaigne, et al., U.S. Pat. No. 8,921,314; and in Castaigne, et al., U.S. Pat. No. 9,173,891 (see also TABLE 2) can be used in combination with the peptides and compositions of the present invention.

As previously mentioned, the compounds of the invention may be administered for the treatment of a cancer in a combination therapy with other suitable treatment modalities. In the case of cancer, such other suitable treatment modalities may include, without limitation, administration of radiation therapy, e.g., gamma radiation therapy. Other suitable treatment modalities may include, for example, administering to a patient in combination with an anticancer vaccine, an anticancer immunotherapy agent, anticancer immunomodulatory agent, an additional anticancer therapeutic, or a combination thereof.

Anticancer vaccines may include, for example, Gardasil and Cervarix (prophylactic) and Sipuleucel-T/Provenge (therapeutic). Anticancer immunotherapy agents may include, for example, Alemtuzumab, Ipilimumab, Nivolumab, Pembroli-zumab, Rituximab, Nivolumab, Interferon, and Interleukin. Anticancer immunomodulatory agents may include for example, thalidomide, lenalidommide, and pomalimomide. Additional anticancer therapeutics may include, for example, an alkylating agent, a tubulin inhibitor, a proteasome inhibitor, a topoisomerase inhibitor (I and II), a CHK1 inhibitor, a CHK2 inhibitor, a PARP inhibitor, doxorubicin, epirubicin, vinblastine, etopside, topotecan, bleomycin, temozolomide, gemcitabine, paclitaxel, a nanoparticle albumin-bound paclitaxel (Abraxane), and mytomycin c.

Alkylating agents may be selected from the group consisting of Dacarbazine, Procarbazine, Carmustine, Lomustine, Uramustine, Busulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin. Tubulin inhibitors may be selected from the group consisting of Taxol, Docetaxel, Vinblastin, Epothilone, Colchicine, Cryptophycin, BMS-347550, Rhizoxin, Ecteinascidin, Dolastin 10, Cryptophycin 52, and IDN-5109. Proteasome inhibitors may be selected from the group consisting of Velcade (bortezomib), and Kyprolis (carfilzomib). Topoisomerase I inhibitors may be selected from the group consisting of Irinotecan, Topotecan, and Camptothecins (CPT). Topoisomerase II inhibitors may be selected from the group consisting of Amsacrine, Etoposide, Teniposide, Epipodophyllotoxins, and ellipticine. CHK1 inhibitors may be selected from the group consisting of TCS2312, PF-0047736, AZ07762, A-69002, and A-641397. PARP inhibitors may be selected from the group consisting of Olaparib, ABT-888, (veliparib), KU-59436, AZD-2281, AG-014699, BSI-201, BGP-15, INO-1001, ONO-2231 and the like.

Through combination therapy, reduction of adverse drug reaction and potentiation of anticancer activity are achievable by the combined effects of anticancer agents having different mechanisms of action, including reduction of the non-sensitive cell population; prevention or delaying of occurrence of drug resistance; and dispersing of toxicity by means of a combination of drugs having different toxicities.

When an anti-cancer agent used in combination has a particular medication cycle, it is preferable to establish an appropriate medication cycle for the compound of formulas I, II and/or III, and such anti-cancer agent, so that the desired effects are attained. Specifically, the frequency of administration, dosage, time of infusion, medication cycle, and the like, may be determined properly according to individual cases, considering the kind of anticancer agent, state of the patients, age, gender, etc.

In using the combination therapy of the present invention, the same dose as that usually given as a monotherapy or a slightly reduced dose (for example, 0.10-0.99 times the highest dose as a single agent) may be given through a normal administration route.

The methods of the present invention will normally include medical follow-up to determine the therapeutic or prophylactic effect brought about in the patient undergoing treatment with the compound(s) and/or composition(s) described herein. Efficacy of the methods may be assessed on the basis of tumor regression, e.g., reducing the size and/or number of neoplasms, inhibition of tumor metastasis, decrease in a serological marker of disease, or other indicator of an inhibitory or remedial effect.

### Example 23: Modulation of the TREM-1 Pathway in Combination-Therapy Treatment Regimen in a Mouse Model of Pancreatic Cancer

In order to demonstrate that when added to a standard chemotherapy regimen, modulators of the TREM-1/DAP-12 signaling pathway are synergistically effective in inhibiting PC growth and improving survival, animal efficacy studies were performed in human PANC-1 xenograft mouse model of PC using 5-6 week old female athymic nude-Foxn1^{nu} mice obtained from Envigo (formerly Harlan, Inc.) using the standard, well known in the art methods as described in (Shen and Sigalov 2017).

GF9-loaded sSLP that contain an equimolar mixture of PE22 and PA22 (GF9-sSLP) were synthesized using the sodium cholate dialysis procedure, purified and characterized as described herein and previously in (Shen and Sigalov 2017, Shen and Sigalov 2017). In a subset of experiments, 1,2-dimyristoyl-sn-glycero-3-phosphoethanolamine-N-(lissamine rhodamine B sulfonyl) was added to reaction mixtures to prepare rhodamine B (rho-B)-labeled GF9-sSLP as described in (Shen and Sigalov 2017, Shen and Sigalov 2017).

Mice were implanted subcutaneously into the right flank with PANC-1 cells in equal parts of serum-free growth medium and Matrigel. Mice were monitored daily and tumor measurements were taken along the length and width using Vernier calipers twice weekly until sacrifice. Tumor volumes were calculated using a modified ellipsoidal formula: (Length×Width²)/2. After tumors in PANC-1-bearing mice reached a volume of 150-200 mm³, mice were randomized into groups and intraperitoneally (i.p.) administered with either vehicle (once daily 5 times per week, 5qw), GF9-sSLP (once daily 5 times per week, 5qw), 100 mg/kg GEM and 10 mg/kg ABX (days 1, 4, 8, 11, 15) or GF9-sSLP (once daily 5 times per week, 5qw) in combination with 100 mg/kg GEM and 10 mg/kg ABX (days 1, 4, 8, 11, 15) (Black triangles). Treatment with GF9-sSLP persisted for 28 days. Body weight were measured. Mean tumor volumes were calculated. On the day 88, tumor volumes were compared between the GEM+ABX-treated and GF9-sSLP+GEM+ABX-treated groups. Survival was evaluated using Kaplan-Meier survival curves.

This example demonstrated that in combination-therapy treatment regimen modulation of the TREM-1/DAP-12 signaling pathway using GF9-sSLP has a synergistic therapeutic effect in terms of significant tumor growth inhibition and substantial (up to 3-fold) improvement of survival rate as compared to standalone TREM-1 therapy and chemotherapy. This example further demonstrated that GF9-sSLP therapy standalone or in combination with a standard chemotherapy is well tolerated by a long term-treated cancer mice. This example further demonstrated that well tolerable TREM-1 therapy can be used in combination with other anticancer therapies as an induction therapy and then, standalone as a maintenance therapy.

### Example 24: Modulation of the TREM-1 Pathway in Combination-Therapy Treatment Regimen in a Mouse Model of Liver Cancer

In order to demonstrate that when added to a standard immunotherapy regimen, modulators of the TREM-1/DAP-12 signaling pathway are synergistically effective in inhibiting hepatocellular carcinoma (HCC, liver cancer) growth and improving survival, the experiments can be conducted analogously to those described in (Wu et al. 2019) using 8 weeks old C57BL/6 mice.

GF9, GF9-sSLP, TREM-1/TRIOPEP and TREM-1/TRIOPEP-sSLP can be synthesized, purified and characterized as described herein and previously in (Shen and Sigalov 2017, Shen and Sigalov 2017). In a subset of experiments, 1,2-dimyristoyl-sn-glycero-3-phosphoethanolamine-N-(lissamine rhodamine B sulfonyl) can be added to reaction mixtures to prepare rhodamine B (rho-B)-labeled GF9-sSLP or TREM-1/TRIOPEP-sSLP as described in (Shen and Sigalov 2017, Shen and Sigalov 2017).

A total of 25 μL mixture of PBS and Basement Membrane Matrix (with the ratio of 1:1, Matrigel) containing 0.5×10⁶ Hepa 1-6 cells are injected into left liver lobe of male C57BL/6 mice to establish the orthotopic model, and both flanks to develop subcutaneous tumor-bearing model, respectively. After inoculation, GF9, GF9-sSLP, TREM-1/TRIOPEP or TREM-1/TRIOPEP-sSLP in 200 μL PBS are i.p. injected once a day. Treatments with anti-PD-L1 or isotype antibody (BioXcell, West Lebanon, N.H.) are conducted in 3, 6, 9 days (20 mg/kg i.p) after implantation. Tumor volumes are calculated according to the modified ellipsoidal formula V=1/2 (length×width²).

For immunohistochemistry or immunofluorescence analysis, the 3 μm tissue sections are subjected to antigen retrieval in an induction cooker for 25 min in EDTA buffer (pH 9.0). Followed by treatment with Goat Serum at 37° C. for 40 min, tissue sections are incubated with the following antibodies: HIF-1α (ab2185), TREM-1 (11791-1-AP, Proteintech, Rosemont, USA), TREM-1 (NBP2-11977, Novus Biologicals, Littleton, Colo.), CD68 (ab955), CD8 (ab203035, ab93278), Foxp3 (ab20034) at 4° C. overnight. Antibodies are from Abcam (Cambridge, Mass.), unless otherwise indicated. For immunofluorescence, slides are incubated with Alexa Fluor 488 and 594 secondary antibodies (A11034, A21125, Invitrogen, Carlsbad, Calif., USA). Immunostaining is visualized under immunofluorescent microscopy and evaluated by Image-Pro Plus software (Media Cybernetics, Silver Springs, Md.). TUNEL Apoptosis assay kit (C1086, Beyotime Biotechnology, Shanghai, China) is performed to detect apoptosis in the specimens according to the manufacturer's instructions. For immunoblotting, protein is extracted (Minute SN-001, Invent Biotechnologies, Inc, Beijing, China) and through 8%-15% SDS-PAGE electrophoresis, transferred to PVDF membranes, and incubated with antibodies against ERK (4695), phospho-ERK (4370), P38 (8690), phosphor-P38 (4511), NF-κβ P65 (8242), phosphor-P65 (3033), IKβα (4814), phosphor-IKβα (2859), S6 (2217). All the primary antibodies are from Cell Signaling Technology (Danvers, Mass.). H1RP-conjugated anti-mouse or anti-rabbit antibody is used as secondary antibody and the antigen-antibody reaction is visualized by enhanced chemiluminescence assay (ECL, Thermo, Waltham, Mass.). For the nuclear protein extraction, cells were subjected to the nuclear protein separation kit (78833, Thermo).

It is anticipated that TREM-1+ TAMs endow HCC with resistance to anti-PD-L1 therapy and the ability to induce CD8+ T-cells exhaustion, implying that a pathway, not the PD-L1/PD-1 axis, plays a substantial role in the process by which TREM-1+ TAMs induce CD8+ T-cells exhaustion in HCC. It is further anticipated that modulation of the TREM-1/DAP-12 signaling pathway using GF9, GF9-sSLP, TREM-1/TRIOPEP and TREM-1/TRIOPEP-sSLP abrogate immunosuppression and PD-L1 blockade resistance and in the combination-therapy treatment regimen with anti-PD-L1 treatment significantly attenuate tumor growth and improve mouse survival.

### Exemplary Prediction of Response to TREM-1 Inhibitory Treatment.

Medical oncologists currently cannot generally predict which patients will or will not respond to a proposed anticancer treatment. Often only a percentage of patients will respond favorably to a particular anticancer treatment. TREM-1 inhibitory therapy targets the TREM-1/DAP-12 signaling pathway which is a novel therapeutic target on the horizon of cancer. Accordingly, there is a great need in the art to identify patient responsiveness to this particular anticancer therapy used as standalone therapy or in combinations with other anticancer treatments.

In certain embodiments, the present invention provides for a method of predicting response of the subject to the treatment by using the modulators of TREM-1/DAP-12 signaling pathway in standalone or combination-therapy regimen by determining the expression of CSF-1, CSF-1R, IL-6, TREM-1 and/or number of CD68-positive TAMs or a combination thereof in a biological sample of the subject.

In some embodiments, the invention provides for a method of diagnosing cancer in which myeloid cells are involved or recruited in the subject and/or predicting response of the subject to the treatment by using the modulators of TREM-1/DAP-12 signaling pathway in standalone or combination-therapy regimen by imaging at least a portion of the patient and detecting the labeled probe conjugated to at least one modulator capable of binding TREM-1 described herein.

Exemplary Analysis of CSF-1, CSF-1R, IL-6, TREM-1, sTREM-1 and CD68 Markers.

As disclosed in U.S. Pat. No. 8,021,836 and described in (Groblewska et al. 2007, Mroczko et al. 2007, Strojnik et al. 2009, Skrzypski et al. 2013, Pei et al. 2014, Richardsen et al. 2015, Kuemmel et al. 2018), CSF-1 (M-CSF), IL-6, CSF-1R, and TREM-1 levels in serum and tumor samples as well as serum levels of sTREM-1 and CD68 (macrophage marker) expression level in tumor samples and combinations thereof can be used as prognostic factors to predict cancer progression and mortality in patients with different types of cancer including but not limited to, human glioma, lung cancer, pancreatic cancer, breast cancer, colorectal cancer and others. As described herein, the TREM-1/DAP-12 signaling pathway is involved in production of these cytokines and growth factors (See FIG. 1). As further described herein, TREM-1 inhibition in cancer mice results in reduction of serum and tissue levels of IL-6, CSF-1 and TREM-1 as well as in suppression of intratumoral macrophage infiltration (See FIGS. 2, 11-13). As further described herein, in cancer mice, response to TREM-1 inhibitory treatment correlates with the intratumoral macrophage content (TAMs), as measured by macrophage marker: the higher is a basal TAM content in the tumor, the better is response to the treatment with modulators of the TREM-1/DAP-12 signaling pathway (See FIG. 10).

In certain embodiments, the present invention provides for a method of predicting response of the subject to the TREM-1 inhibitory anticancer treatment using the modulators of TREM-1/DAP-12 signaling pathway in standalone or combination-therapy regimen by: (a) obtaining a biological sample from the subject including but not limited to, blood, plasma, serum, tumor tissues, tumor biopsies, etc.; (b) determining the expression/level of CSF-1, CSF-1R, IL-6, TREM-1, sTREM-1 and/or number of CD68-positive cells including TAMs or a combination thereof using analytical methods, techniques and procedures described herein, disclosed in U.S. Pat. No. 8,021,836 and described in (Groblewska et al. 2007, Mroczko et al. 2007, Strojnik et al. 2009, Skrzypski et al. 2013, Pei et al. 2014, Sigalov 2014, Richardsen et al. 2015, Shen and Sigalov 2017, Shen and Sigalov 2017, Kuemmel et al. 2018, Rojas et al. 2018, Tornai et al. 2019), wherein the higher is the expression of CSF-1, CSF-1R, IL-6, TREM-1, sTREM-1 or the higher is number of CD68-positive TAMs or a combination thereof, the better the patient is predicted to respond to a TREM-1 inhibitory therapy that involves the modulators of the TREM-1/DAP-12 signaling pathway.

### Exemplary Imaging of TREM-1 Expression.

As described herein and in (Rojas et al. 2018), TREM-1 inhibitory therapy using the modulators of the TREM-1/DAP-12 signaling pathway results in reduction of tissue TREM-1 expression as measured by Western Blot (See FIG. 13). Another way to evaluate the TREM-1 expression level is to use imaging (visualization) techniques and procedures.

In one embodiment, FIG. 3 shows that the fluorescently labeled TREM-1/TRIOPEP peptide GE31 delivered to macrophages by the SLP particles colocalizes with TREM-1 expressed on these cells. See also (Rojas et al. 2018). In certain embodiments, the capability of the modulators of the TREM-1/DAP-12 signaling pathway described herein, including but not limited to, anti-TREM-1 blocking antibodies and fragments thereof, TREM-1 inhibitory SCHOOL peptides (e.g., GF9) and trifunctional TREM-1 inhibitory peptides including but not limited to, GA31 and GE31, to colocalize with TREM-1 can be used to visualize (image) this receptor and evaluate its expression/level in the areas of interest. In one embodiment, for this purpose, an imaging probe (e.g. [⁶⁴Cu], see TABLE 3) can be conjugated to the peptide sequences, GE31 (GFLSKSLVFPYLDDFQKKWQEEM(O)ELYRQKVE, M(O), methionine sulfoxide) (SEQ ID NO. 27) and/or GA31 (GFLSKSLVFPLGEEM(O)RDRARAHVDALRTHLA (M(O), methionine sulfoxide) (SEQ ID NO. 26). In one embodiment, methionine residues of the peptides GE31 (GFLSKSLVFPYLDDFQKKWQEEMELYRQKVE) (SEQ ID NO. 25) and GA31 (GFLSKSLVFPLGEEMRDRARAHVDALRTHLA) (SEQ ID NO. 24) are unmodified. In one embodiment, imaging (visualization) of TREM-1 levels using the labeled modulators described herein and the PET and/or other imaging techniques can be used to diagnose GBM and/or to select and monitor novel GBM therapies as disclosed in WO 2017083682A1 and described in (Johnson et al. 2017, Liu et al. 2019). In certain embodiments, imaging (visualization) of TREM-1 levels can be used to diagnose other TREM-1-related diseases and conditions as well as to monitor novel therapies for these diseases and conditions.

In some embodiments, an imaging probe is selected from the group comprising Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol, or a combination thereof.

In one embodiment, the imaging agent is a GBCA for MRI. In one embodiment, the imaging agent is a [⁶⁴Cu]-containing imaging probe for imaging systems such as PET imaging systems (and combined PET/CT and PET/MRI systems). In one embodiment, the peptides and compositions of the invention are used in combinations thereof. In one embodiment, the peptides and compositions of the invention are used in combinations with other anticancer therapeutic agents. In certain embodiments, the modulators and compositions described herein are incorporated into long half-life SLP. In certain embodiments, the modulators and compositions described herein may incorporate into lipopeptide particles (LP) in vivo upon administration to the individual. In certain embodiments, the peptides and compositions of the invention can cross the blood-brain barrier (BBB), blood-retinal barrier (BRB) and blood-tumor barrier (BTB). Thus, in one aspect, the invention provides for a method for suppressing tumor growth in an individual in need thereof by administering to the individual an amount of a TREM-1 inhibitor that is effective for suppressing tumor growth.

As described in (Stukas et al. 2014), systemically administered human apo A-I accumulates in murine brain. It is also known that transcytosis of HDL in brain microvascular endothelial cells is mediated by SRBI (see (Fung et al. 2017)). In certain embodiments, FIG. 18 demonstrates that the SLP described herein may cross the BBB, BRB and BTB, thus delivering their constituents including but not limiting to, GF9, GA31 and GE31 to the areas of interest in the brain, retina and tumor. In certain embodiments, FIG. 18 demonstrates that the fluorescently labeled sSLP described herein may cross the BBB, BRB and BTB, thus delivering their constituents including but not limiting to, GBCA imaging probe to the areas of interest in the brain, retina and tumor. While not being bound to any particular theory, it is believed that the brain-, retina-, and tumor-penetrating capabilities of these SLP can be mediated by interaction of SRBI with the amino acid sequences that correspond to the sequences of human apo A-I helices 4 and/or 6 (see e.g. (Liadaki et al. 2000, Liu et al. 2002)). In certain embodiments, these capabilities of the modulators and compositions described herein can be used to diagnose, treat and/or prevent cancers (e.g. brain cancer and retina cancer), where delivery of the peptides and compositions of the invention to the brain, retina and/or tumor is needed.

In certain embodiments, the invention provides for a diagnostic method of detecting TREM-1 expression levels in an individual with cancer by: (a) administering to the individual the modulators of TREM-1 transmembrane signaling having an affinity for TREM-1 and an imaging probe in a detectably effective amount; (b) imaging at least a portion of the patient; (c) detecting the labeled probe, wherein the location of the labeled probe corresponds to at least one symptom of the myeloid cell-related cancer condition and correlates with the TREM-1 expression levels and the higher the levels are, the better the patient is predicted to respond to a TREM-1 inhibitory therapy using the modulators of the TREM-1/DAP-12 signaling pathway as standalone therapy or in combinations with other anticancer treatments.

### Results

SCHOOL TREM-1 inhibitory GF9 sequences exhibit single-agent antitumor activity and prolong survival in BxPC-3, AsPC-1, and Capan-1 xenograft mouse models. Previously, we reported that oxidation of apo A-I or its peptides H4 and H6 significantly enhances targeted delivery of SCHOOL TREM-1 inhibitory GF9 sequences or imaging agents incorporated into HDL-mimicking lipopeptide complexes to macrophages in vitro and in vivo. (Sigalov 2014, Sigalov 2014, Shen et al. 2015, Shen et al. 2016) We also demonstrated that free and HDL-bound GF9 exhibits single-agent antitumor activity in H292 and A549 xenograft models of NSCLC and hypothesized that this TAM-targeted antitumor strategy is cancer type-independent. (Sigalov 2014) This prompted us to extend our previous work and test the in vivo efficacy of GF9, GF9-HDL and GA31+GE31 in an equimolar ratio (GA/E31)-HDL in BxPC-3, AsPC-1, and Capan-1 xenograft models of PC in nude mice.

When administered daily at a dose of 25 mg/kg, free GF9 showed antitumor efficacy in all three models studied (FIG. 2A), with the effect most pronounced in the Capan-1 model (31% T/C) compared with the BxPC-3 and AsPC-1 models (41 and 56% T/C, respectively). The observed antitumor effect of 25 mg/kg GF9 is dose-dependent and specific: administration of GF9 at 2.5 mg/kg or a control peptide GF9-G at 25 mg/kg did not affect tumor growth.

To test whether targeted delivery of GF9 to macrophages by formulation of GF9 into macrophage-targeted lipopeptide complexes of either discoidal (dHDL) or spherical (sHDL) reduces the effective peptide dose, GF9-dHDL and GF9-sHDL were administered daily at 2.5 mg of GF9/kg. Despite a 10-fold decrease in administration dose of GF9, the observed therapeutic effect of GF9-HDL was comparable (42, 40, and 28% T/C as observed for GF9-dHDL in AsPC-1, BxPC-3, and Capan-1, respectively) or even better (26% T/C as observed for GF9-sHDL in BxPC-3) than that observed for GF9 at 25 mg/kg (FIG. 2A). Further, we have previously shown that peptides GE31 and GA31 with sequences from GF9 and apo A-I helices 4 and 6 are able to perform three functions: assist in the self-assembly of HDL, target HDL to macrophages and silence the TREM-1 signaling pathway in mice with CIA. (Shen and Sigalov 2017) To address the question of whether these SCHOOL TREM-1 inhibitory GF9 sequences formulated into HDL-mimicking macrophage-targeted lipopeptide complexes can provide antitumor efficacy in vivo, we administered either GA/E31-dHDL or GA/E31-sHDL at a dose equivalent to 4 mg/kg GF9 and found that GA/E31-HDL inhibited tumor growth in all three xenograft models (FIG. 3A) with activity comparable or even better (19 and 21% T/C as observed for GA/E31-dHDL in BxPC-3 and Capan-1, respectively) than that observed for GF9-HDL at 2.5 mg/kg (FIG. 2A). Kaplan-Meier survival curves demonstrated that treatment with free or HDL-bound SCHOOL TREM-1 inhibitory GF9 sequences significantly prolonged survival relative to vehicle control in all three xenograft models of PC studied (FIG. 4). Collectively, these findings suggest that incorporation of GF9 alone or as a part of GE31 and GA31 peptides into macrophage-specific lipopeptide complexes reduces the effective peptide dose up to 10-fold probably due to improved pharmacokinetic parameters of the peptide and its targeted delivery. In addition, the antitumor activity demonstrated by GA/E31-HDL (FIGS. 3 and 4) further confirms our previous findings regarding the multifunctionality of these peptide sequences. (Shen and Sigalov 2017) It should be also noted that in all xenograft mouse models of PC studied, daily administration of free or HDL-bound SCHOOL TREM-1 inhibitory GF9 sequences for 5 consecutive days per week for more than 4 weeks did not cause any loss in the animal weight (FIGS. 2B and 3B), and there was no obvious sign of toxicity during the course of the treatment. This further confirms our previous findings that free and HDL-bound GF9 are well-tolerated by healthy mice and H292 and A549 tumor-bearing mice. (Sigalov 2014) In summary, these data collectively not only provide the first in vivo experimental evidence of the potential involvement of TREM-1-regulated pathway in PC, thereby implicating the potential of TREM-1 inhibitors as novel antitumor agents for the treatment of PC, but also support the previously predicted cancer type-independent mechanisms of antitumor activity of SCHOOL TREM-1 inhibitory GF9 sequences. (Sigalov 2014) Formulation of these sequences into macrophage-specific self-assembling lipopeptide complexes that mimic human HDL substantially increases their therapeutic efficacy probably because of targeted delivery and/or the half-life extension of the peptides in circulation afforded by this strategy.

SCHOOL TREM-1 inhibitory GF9 sequences suppress macrophage infiltration into the tumor. The in vivo biologic effects of SCHOOL TREM-1 inhibitory GF9 sequences were further addressed by histological and immunohistochemical (IHC) studies. To investigate immune infiltration into the tumor microenvironment and address whether macrophages were reduced in BxPC-3-, AsPC-1-, and Capan-1-bearing mice treated with GF9, GF9-HDL and GA/E31-HDL, we performed IHC analysis using the murine macrophage marker F4/80. In vehicle-treated mice, IHC analysis revealed that intratumoral macrophage infiltration depended on the PC xenograft line: significantly higher macrophage infiltration (up to 20%) was observed in BxPC-3 and Capan-1 tumors compared with that in AsPC-1 tumors (less than 5%) (data not shown). To establish a correlation between the anticancer effect of the TREM-1 treatments used and TAM content, we plotted the calculated anticancer activity (% T/C) from groups of BxPC-3-, AsPC-1-, and Capan-1-bearing mice treated using free and HDL-bound SCHOOL TREM-1 inhibitory GF9 sequences against the intratumoral macrophage content and observed that the higher the intratumoral macrophage content, the higher was the antitumor activity of the tested SCHOOL TREM-1 inhibitory GF9 sequences (FIG. 5A). We also found that treatment with SCHOOL TREM-1 inhibitory GF9 sequences substantially reduced macrophage content in tumors of BxPC-3-bearing mice compared with control (FIGS. 5B and 5C). One may suggest that testing a tumor for its inflammation status can help to identify those patients who will better respond to TREM-1-targeted therapy. In summary, these findings show for the first time that TREM-1 inhibition reduces macrophage infiltration into xenograft tumors.

SCHOOL TREM-1 inhibitory GF9 sequences inhibit the release of proinflammatory cytokines and M-CSF. M-CSF and proinflammatory cytokines such as IL-1α and IL-6 are involved in tumor angiogenesis and PC invasiveness. (Groblewska et al. 2007, Kubota et al. 2009, Tjomsland et al. 2011, Yako et al. 2016) Previously, we observed that TREM-1 inhibition using free and HDL-bound SCHOOL TREM-1 inhibitory GF9 sequences reduces the production of proinflammatory cytokines and M-CSF in septic mice(Sigalov 2014) and mice with CIA. (Shen and Sigalov 2017) In this study, to further elucidate the molecular mechanisms underlying the observed anticancer effect of SCHOOL TREM-1 inhibitory GF9 sequences, we investigated whether TREM-1 inhibition using GF9, GF9-HDL and GA/E31-HDL affects the release of proinflammatory cytokines and M-CSF in BxPC-3-, AsPC-1-, and Capan-1-bearing mice. We analyzed serum cytokine levels on study days 1 and 8 and found that administration of free GF9 at 25 mg/kg and GF9-sHDL at 2.5 mg/kg inhibits the production of IL-1α (except of AsPC-1), IL-6, and M-CSF compared with vehicle-treated mice (FIG. 6; shown for GF9 and GF9-sHDL). Similar data were obtained for GA/E31-HDL (not shown). The effect is dose-dependent and specific: GF9 at 2.5 mg/kg and GF9-G at 25 mg/kg did not affect the release of either cytokines or M-CSF (not shown). Collectively, these data indicate that inhibition of the TREM-1 signaling pathway using free and HDL-bound SCHOOL TREM-1 inhibitory GF9 sequences reduces the release of proinflammatory cytokines and M-CSF in experimental PC.

### Discussion

To the best of our knowledge, this study is the first report showing the in vivo efficacy of novel, ligand-independent SCHOOL TREM-1 inhibitory GF9 sequences in free form (GF9) and formulated into macrophage-specific lipopeptide complexes (GF9-HDL and GA/E31-HDL) in PC. This not only extends our previous observations that free and HDL-bound GF9 exhibit the in vivo anticancer efficacy in NSCLC(Sigalov 2014) but, importantly, suggests that the therapeutic effect of our TREM-1-targeted treatment is cancer type-independent. The major findings in the present study are that administration of GF9, GF9-HDL, and GA/E31-HDL results in a strong antitumor effect achieving an optimal T/C value of 19% depending on the xenograft and formulation used and persisting even after treatment was halted (FIGS. 2A and 3A). We also demonstrate that mice treated with these TREM-1 inhibitors show substantially prolonged survival in comparison to the control (FIG. 4). These and our previous data(Sigalov 2014) are in line with the observed three-fold increase in the 4-year survival rate in NSCLC patients with low TREM-1 expression on TAMs compared with those with high TREM-1 expression. (Ho et al. 2008) Therefore, the obtained results provide significant proof of concept that SCHOOL TREM-1 inhibitory GF9 sequences that can inhibit TREM-1 in a ligand-independent manner may have potential as a new TAM-targeted treatment for solid tumors. This treatment can be potentially used as a stand-alone therapy or as a component of combinational therapy.

Interestingly, GF9, GF9-HDL, and GA/E31-HDL all inhibit tumor growth and prolong survival in BxPC-3, AsPC-1, and Capan-1 xenografts (FIGS. 2, 3, and 4) suggesting that GF9 can reach its intramembrane site of action from either outside (free GF9, Route 1, FIG. 1B) or inside the cell (GF9-HDL and GA/E31-HDL, Route 2, FIG. 1i). Further, the beneficial antitumor effect of TREM-1 inhibitory GF9 sequences either in free or HDL-bound form continues after cessation of treatment (FIGS. 2 and 3). This suggests that once delivered to its site of action, GF9 or GF9-containing peptide sequence can remain in the membrane and inhibit TREM-1. In vivo half-life extension of GF9 incorporated into HDL as GF9 alone or as a part of GE31 and GA31 peptides (Zu Shen and Alexander Sigalov, unpublished data) may also contribute to the observed effect: while the peptide half-life in vivo is short, typically a few minutes, (Gupta et al. 2013) discoidal (or nascent) HDL and sHDL are known to have half-lives of 12-20 hrs and 4-5 days, respectively. (Furman et al. 1964).

TAMs facilitate a microenvironment that promotes tumor development and are an important drug target for cancer therapy. (Condeelis et al. 2006, Lin et al. 2006) An increased level of macrophage infiltration into tumors correlates with increased angiogenesis and poor prognosis. (Condeelis and Pollard 2006) By promoting tumor angiogenesis, TAMs play an important role in the tumor progression to malignancy. Inhibition of intratumoral macrophage infiltration delays the angiogenic switch and the malignant transition. (Lin et al. 2006) In this study, we found that blockade of TREM-1 inhibits macrophage infiltration in BxPC-3 tumors (FIG. 5). Interestingly, the antitumor activity of SCHOOL TREM-1 inhibitory GF9 sequences as assessed by tumor size and survival of AsPC-1-, BxPC-3-, and Capan-1-bearing mice correlated with the intratumoral macrophage content observed in these xenograft models treated with vehicle alone. This suggests that TAM content may represent a biomarker that may help to identify those patients who will better respond to TREM-1-targeted therapy. This biomarker could be also used as a criterion for either including or excluding trial participants.

Myeloid cells are known to contribute directly to tumor angiogenesis and lymphangiogenesis by secreting multiple angiogenic factors including VEGF and M-CSF, which play a key role in cancer pathogenesis. (Kubota et al. 2009, Zumsteg et al. 2009) Thus, one of the potential mechanisms underlying the in vivo anticancer activity of TREM-1 inhibitors observed in this study can be partly mediated through M-CSF inhibition, resulting in suppression of not only macrophage infiltration into tumor sites but also angiogenesis. In mice with osteosarcoma, inhibition of M-CSF selectively suppresses tumor angiogenesis and lymphangiogenesis. (Kubota et al. 2009) In line with these findings, our preliminary data (Zu Shen and Alexander Sigalov, unpublished observations) indicate that treatment with SCHOOL TREM-1 inhibitory GF9 sequences substantially reduced the number of blood vessels within the tumor as revealed by IHC analysis of the tumors from control and treated animals for microvessel density using CD31, or platelet endothelial cell adhesion molecule-1 (PECAM-1), as a marker to evaluate neovascularization in tumor xenografts. (Wang et al. 2008) Importantly, in contrast to blockade of VEGF that damages healthy vessels and promotes rapid vascular regrowth, (Pieramici et al. 2008) continuous inhibition of M-CSF does not affect healthy vascular and lymphatic systems outside tumors and is currently considered as a promising therapeutic strategy for targeting angiogenesis in cancer and ocular neovascular diseases. (Kubota et al. 2009) In this study, we observed reduction of serum M-CSF but not VEGF in human PC tumor-bearing mice treated with either free or HDL-bound SCHOOL TREM-1 inhibitory GF9 sequences compared to vehicle-treated mice (FIG. 6) or mice treated with GF9-G (not shown). Consistent with a recent report, (Kubota et al. 2009) interruption of M-CSF inhibition via cessation of TREM-1 treatment does not result in rapid tumor regrowth in all xenografts studied (FIGS. 2A and 3A). Our current results are also in line with our previous data that demonstrate that in mice with CIA, treatment with SCHOOL TREM-1 inhibitory GF9 sequences results in reduced serum levels of M-CSF. (Shen and Sigalov 2017) This also correlates well with our recent findings that in mice with oxygen-induced retinopathy (OIR), treatment with SCHOOL TREM-1 inhibitory GF9 sequences significantly reduces retinal expression of TREM-1 and prevents retinal neovascularization (Modesto Rojas, Zu Shen, Ruth Caldwell, Alexander Sigalov; unpublished data). Collectively, these data strongly support the hypothesis that TREM-1 blockade-mediated inhibition of M-CSF can contribute to the anticancer activity of TREM-1 inhibitors observed in this and a previous study. (Sigalov 2014)

Our study shows that blockade of TREM-1 specifically suppresses key cytokines such as IL-1α, IL-6 and M-CSF, which are upregulated in PC and contribute to poor prognosis. (Kumari et al. 2016, Yako et al. 2016) In patients with PC, high IL-1α expression has been found to correlate poorly with clinical outcome and the patient's survival time. (Tjomsland et al. 2011) By targeting CAFs, IL-1α plays a pivotal role in sustaining the PC tumor inflammatory microenvironment that supports tumor growth and progression and the recruitment of leukocytes such as macrophages. (Esposito et al. 2004, Tjomsland et al. 2011) Thus, the suppression of IL-1α observed in this study is likely due to blockade of TREM-1 expressed on TAMs and subsequent disruption of the TAM-CAF network (FIG. 1). Another key player in the tumor microenvironment, IL-6, promotes tumorigenesis by regulating apoptosis, survival, proliferation, angiogenesis, invasiveness and metastasis, and, most importantly, metabolism. (Kumari et al. 2016) These considerations, together with the fact that IL-1α plays an important role in tumor-mediated angiogenesis, TREM-1 inhibition-mediated reduction of IL-1α and IL-6 may represent another mechanism of the antitumor activity of TREM-1 inhibitory GF9 sequences demonstrated in the present study.

One of the interesting opportunities that this study offers is to test the hypothesis that a combinatorial therapy for treating PC that includes first-line cytotoxic therapy (e.g., gemcitabine, GEM) and TREM-1 treatment that targets the tumor inflammatory microenvironment can synergistically improve survival of PC patients but also reduce recurrence risk. Interestingly, it was recently reported that GEM treatment increases TAM infiltration into PDAC tumors. (Mitchem et al. 2013) On the other hand, while targeting TAMs via inhibition of M-CSF (CSF-1) receptor modestly slows tumor growth but decreases the number of tumor-initiating cells (TIC) in pancreatic tumors, combination of GEM with M-CSF receptor inhibitors synergistically increases chemotherapeutic efficacy, dramatically slows PC progression and blocks metastasis by the combined action of reducing TIC content. (Mitchem et al. 2013) Here, it should be noted that compared to normal pancreas, PDAC tissues are known to express 3.7-fold more scavenger receptor (SR) class B member 1 (SR-B1), (Julovi et al. 2016) which is normally expressed in the liver and functions as a receptor for HDL. Thus, in addition to targeting macrophages (including TAMs) by receptor (likely via SR class A, SR-A)-mediated uptake that involves methionine-sulfoxidized sites of the apo A-I helices 4 and 6, HDL-like lipopeptide complexes may also target PDAC cells via SR-B1-mediated uptake that involves potential SR-B1 epitopes located on the apo A-I helices 4 and 6. (Liu et al. 2002) This could allow the use of this lipopeptide platform to deliver not only TREM-1 therapy but also cytotoxic agents in a combinatorial therapy for PC. Further studies are needed to confirm this hypothesis.

Intriguingly, like other peptides that utilize the SCHOOL approach, (Wang et al. 2002, Sigalov 2008, Shen and Sigalov 2016) TREM-1 inhibitory GF9 sequences self-insert into the plasma membrane from either outside or inside the cell (FIG. 1B, Routes 1 and 2, respectively) and disconnect TREM-1 from DAP-12 (Sigalov 2014, Shen and Sigalov 2017) in a manner similar to that used by different viruses to suppress the host immune response. (Sigalov 2009, Shen and Sigalov 2016) Together with the present study, this further supports our unifying hypothesis (Sigalov 2009, Shen and Sigalov 2016) that these viral strategies developed and optimized during millions of years of evolution of virus-host interactions can be rationally used in the development of novel therapies.

### Conclusion.

The present study demonstrates that novel SCHOOL TREM-1 inhibitory GF9 sequences potently inhibit PC tumor growth and prolong survival in human PC tumor-bearing mice. To our knowledge, this study is the first to demonstrate the potential role of TREM-1 as a therapeutic target in PC treatment and to suggest suppression of the specific inflammatory response through silencing the TREM-1-mediated signaling pathway using TREM-1 peptide inhibitors designed using the SCHOOL approach as a novel therapeutic strategy against PC. Future studies are needed for testing these inhibitors in combination with chemotherapy and radiotherapy aiming to overcome the intrinsic and acquired resistance of PC cells, to enhance the treatment efficacy, and to reduce PC patient's short- and long-term risks of recurrence and progression. Our data provide further in vivo evidence in support of M-CSF- and cytokine-mediated molecular mechanisms underlying the tumor growth-inhibiting effect of these inhibitors in free or HDL-bound form observed in PC (this study) and NSCLC. (Sigalov 2014) This further suggests that SCHOOL TREM-1 inhibitory GF9 sequences have a cancer type-independent, therapeutically beneficial antitumor activity and can be potentially used as a stand-alone therapy or as a component of combinational therapy for PC, NSCLC, and potentially other solid tumors (e.g., breast and colon cancer). In conjunction with the ability of the HDL-like lipopeptide complexes used in this study to cross the blood-brain barrier (Zu Shen and Alexander Sigalov, unpublished observations), this therapy can be potentially used to treat brain tumors such as glioblastoma, as well.

TABLE 2

| | Exemplary Trifunctional |
|---|---|
| ## | Peptides and Compositions |
| 1 | GFLSKSLVF |
| 2 | GFLSKSLVFPLGEEMRDRARAHVDALRTHLA |
| 3 | GFLSKSLVFPYLDDFQKKWQEEMELYRQKVE |
| 4 | GFLSKSLVFPLGEEM(O)RDRARAHVDALRTHLA |
| 5 | GFLSKSLVFPYLDDFQKKWQEEM(O)ELRQKVE |
| 6 | GFLSKSLVFPLGEEMRDRARAHVDALRTHLARGD |
| 7 | GFLSKSLVFPYLDDFQKKWQEEMELYRQKVERGD |
| 8 | [⁶⁴Cu]GFLSKSLVFPLGEEM(O)RDRARAHVDALRTHLA |
| 9 | [⁶⁴Cu]GFLSKSLVFPYLDDFQKKWQEEM(O)ELRQKVE |
| 10 | LQEEDAGEYGCMPLGEEM(O)RDRARAHVDALRTHLA |
| 11 | LQEEDAGEYGCMPYLDDFQKKWQEEM(O)ELYRQKVE |
| 12 | [⁶⁴Cu]LQEEDAGEYGCMPLGEEM(O)RDRARAHVDALRTHLA |
| 13 | [⁶⁴Cu]LQEEDAGEYGCMPYLDDFQKKWQEEM(O)ELYRQKVE |
| 14 | LQVTDSGLYRCVIYHPPPLGEEM(O)RDRARAHVDALRTHLA |
| 15 | LQVTDSGLYRCVIYHPPPYLDDFQKKWQEEM(O)ELYRQKVE |
| 16 | [⁶⁴Cu]LQVTDSGLYRCVIYHPPPLGEEM(O)RDRARAHVDAL |
| | RTHLA |
| 17 | [⁶⁴Cu]LQVTDSGLYRCVIYHPPPYLDDFQKKWQEEM(O)ELY |
| | RQKVE |
| 18 | LQEEDAGEYGCMPLGEEM(O)RDRARAHVDALRTHLA |
| 19 | LQEEDAGEYGCMPYLDDFQKKWQEEM(O)ELYRQKVE |
| 20 | [⁶⁴Cu]LQEEDAGEYGCMPLGEEM(O)RDRARAHVDALRTHLA |
| 21 | [⁶⁴Cu]LQEEDAGEYGCMPYLDDFQKKWQEEM(O)ELYRQKVE |
| 22 | LQVTDSGLYRCVIYHPPPLGEEM(O)RDRARAHVDALRTHLA |
| 23 | LQVTDSGLYRCVIYHPPPYLDDFQKKWQEEM(O)ELYRQKVE |
| 24 | [⁶⁴Cu]LQVTDSGLYRCVIYHPPPLGEEM(O)RDRARAHVDAL |
| | RTHLA |
| 25 | [⁶⁴Cu]LQVTDSGLYRCVIYHPPPYLDDFQKKWQEEM(O)ELY |
| | RQKVE |
| 26 | MWKTPTLKYFPLGEEMRDRARAHVDALRTHLA |
| 27 | MWKTPTLKYFPYLDDFQKKWQEEMELYRQKVE |
| 28 | [⁶⁴Cu]MWKTPTLKYFPLGEEMRDRARAHVDALRTHLA |
| 29 | [⁶⁴Cu]MWKTPTLKYFPYLDDFQKKWQEEMELYRQKVE |
| 30 | GARSMTLTVQARQLPLGEEMRDRARAHVDALRTHLA |
| 31 | GARSMTLTVQARQLPYLDDFQKKWQEEMELYRQKVE |
| 32 | [⁶⁴Cu]GARSMTLTVQARQLPLGEEMRDRARAHVDALRTHLA |
| 33 | [⁶⁴Cu]GARSMTLTVQARQLPYLDDFQKKWQEEMELYRQKVE |
| 34 | GVLRLLLFKLPLGEEMRDRARAHVDALRTHLA |
| 35 | GVLRLLLFKLPYLDDFQKKWQEEMELYRQKVE |
| 36 | [⁶⁴Cu]GVLRLLLFKLPLGEEMRDRARAHVDALRTHLA |
| 37 | [⁶⁴Cu]GVLRLLLFKLPYLDDFQKKWQEEMELYRQKVE |
| 38 | LGKATLYAVLPLGEEMRDRARAHVDALRTHLA |
| 39 | LGKATLYAVLPYLDDFQKKWQEEMELYRQKVE |
| 40 | [⁶⁴Cu]LGKATLYAVLPLGEEMRDRARAHVDALRTHLA |
| 41 | [⁶⁴Cu]LGKATLYAVLPYLDDFQKKWQEEMELYRQKVE |
| 42 | YLLDGILFIYPLGEEMRDRARAHVDALRTHLA |
| 43 | YLLDGILFIYPYLDDFQKKWQEEMELYRQKVE |
| 44 | [⁶⁴Cu]YLLDGILFIYPLGEEMRDRARAHVDALRTHLA |
| 45 | [⁶⁴Cu]YLLDGILFIYPYLDDFQKKWQEEMELYRQKVE |
| 46 | IIVTDVIATLPLGEEMRDRARAHVDALRTHLA |
| 47 | IIVTDVIATLPYLDDFQKKWQEEMELYRQKVE |
| 48 | [⁶⁴Cu]IIVTDVIATLPLGEEMRDRARAHVDALRTHLA |
| 49 | [⁶⁴Cu]IIVTDVIATLPYLDDFQKKWQEEMELYRQKVE |
| 50 | FLFAEIVSIFPLGEEMRDRARAHVDALRTHLA |
| 51 | FLFAEIVSIFPYLDDFQKKWQEEMELYRQKVE |
| 52 | [⁶⁴Cu]FLFAEIVSIFPLGEEMRDRARAHVDALRTHLA |
| 53 | [⁶⁴Cu]FLFAEIVSIFPYLDDFQKKWQEEMELYRQKVE |
| 54 | IVIVDICITGPLGEEMRDRARAHVDALRTHLA |
| 55 | IVIVDICITGPYLDDFQKKWQEEMELYRQKVE |
| 56 | [⁶⁴Cu]IVIVDICITGPLGEEMRDRARAHVDALRTHLA |
| 57 | [⁶⁴Cu]IVIVDICITGPYLDDFQKKWQEEMELYRQKVE |
| 58 | IAVAMGIRFIIMVAPLGEEMRDRARAHVDALRTHLA |
| 59 | IAVAMGIRFIIMVAPYLDDFQKKWQEEMELYRQKVE |
| 60 | GNLVRICLGAPLGEEMRDRARAHVDALRTHLA |
| 61 | GNLVRICLGAPYLDDFQKKWQEEMELYRQKVE |
| 62 | [⁶⁴Cu]GNLVRICLGAPLGEEMRDRARAHVDALRTHLA |
| 63 | [⁶⁴Cu]GNLVRICLGAPYLDDFQKKWQEEMELYRQKVE |
| 64 | VMGDLVLTVLPLGEEMRDRARAHVDALRTHLA |
| 65 | VMGDLVLTVLPYLDDFQKKWQEEMELYRQKVE |
| 66 | [⁶⁴Cu]VMGDLVLTVLPLGEEMRDRARAHVDALRTHLA |
| 67 | [⁶⁴Cu]VMGDLVLTVLPYLDDFQKKWQEEMELYRQKVE |
| 68 | LVAADAVASLPLGEEMRDRARAHVDALRTHLA |
| 69 | LVAADAVASLPYLDDFQKKWQEEMELYRQKVE |
| 70 | [⁶⁴Cu]LVAADAVASLPLGEEMRDRARAHVDALRTHLA |
| 71 | [⁶⁴Cu]LVAADAVASLPYLDDFQKKWQEEMELYRQKVE |
| 72 | SIATGMVGALLLLLVVALGIGLFMRPLGEEMRDRARAHVDAL |
| | RTHLA |
| 73 | SIATGMVGALLLLLVVALGIGLFMRPYLDDFQKKWQEEMELY |
| | RQKVE |
| 74 | DIVKLTVYDCIRRRRRRRRRPLGEEMRDRARAHVDALRTHLA |
| 75 | DIVKLTVYDCIRRRRRRRRRPYLDDFQKKWQEEMELYRQKVE |
| 76 | SLRRSSCFGGRMDRIGAQSGLGCNSFRYPLGEEMRDRARAHV |
| | DALRTHLA |
| 77 | SLRRSSCFGGRMDRIGAQSGLGCNSFRYPYLDDFQKKWQEEM |
| | ELYRQKVE |
| 78 | Ptx-GFLSKSLVFPLGEEMRDRARAHVDALRTHLA |
| 79 | Ptx-GFLSKSLVFPYLDDFQKKWQEEMELYRQKVE |
| 80 | Ptx-GFLSKSLVFPLGEEM(O)RDRARAHVDALRTHLA |
| 81 | Ptx-GFLSKSLVFPYLDDFQKKWQEEM(O)ELRQKVE |
| 82 | IVILLAGGFLSKSLVFSVLFAPLGEEMRDRARAHVDALRTHL |
| | A |
| 83 | IVILLAGGFLSKSLVFSVLFAPYLDDFQKKWQEEMELYRQKV |
| | E |
| 84 | GFLSKSLVFGEEMRDRARAHV |
| 85 | GFLSKSLVFGEEM(O)RDRARAHV |
| 86 | GFLSKSLVFWQEEMELYRQKV |
| 87 | GFLSKSLVFWQEEM(O)ELYRQKV |
| 88 | GFLSRSLVFGEEMRDRARAHV |
| 89 | GFLSRSLVFGEEM(O)RDRARAHV |
| 90 | GFLSRSLVFWQEEMELYRQKV |
| 91 | GFLSRSLVFWQEEM(O)ELYRQKV |
| 92 | GLLSKSLVFGEEMRDRARAHV |
| 93 | GLLSKSLVFGEEM(O)RDRARAHV |
| 94 | GLLSKSLVFWQEEMELYRQKV |
| 95 | GLLSKSLVFWQEEM(O)ELYRQKV |
| 96 | GFLSKSLVFGEEMRDRARAHVRGD |
| 97 | GFLSKSLVFWQEEMELYRQKVRGD |
| 98 | GFLSKSLVFPLGEEMRDRARAHVDALRTHLA |
| 99 | GFLSKSLVFPYLDDFQKKWQEEMELYRQKVE |
| 100 | GFLSKSLVFPLGEEM(O)RDRARAHVDALRTHLA |
| 101 | GFLSKSLVFPYLDDFQKKWQEEM(O)ELYRQKVE |
| 102 | GFLSKSLVFPLGEEMRDRARAHVDALRTHLARGD |
| 103 | GFLSKSLVFPYLDDFQKKWQEEMELYRQKVERGD |
| 104 | [⁶⁴Cu]GFLSKSLVFGEEM(O)RDRARAHV |
| 105 | [⁶⁴Cu]GFLSKSLVFWQEEM(O)ELYRQKV |
| 106 | [⁶⁴Cu]GFLSKSLVFPLGEEM(O)RDRARAHVDALRTHLA |
| 107 | [⁶⁴Cu]GFLSKSLVFPYLDDFQKKWQEEM(O)ELYRQKVE |
| 108 | LQEEDAGEYGCMPLGEEM(O)RDRARAHVDALRTHLA |
| 109 | LQEEDAGEYGCMPYLDDFQKKWQEEM(O)ELYRQKVE |
| 110 | [⁶⁴Cu]LQEEDAGEYGCMPLGEEM(O)RDRARAHVDALRTHLA |
| 111 | [⁶⁴Cu]LQEEDAGEYGCMPYLDDFQKKWQEEM(O)ELYRQKVE |
| 112 | LQEEDAGEYGCMGEEM(O)RDRARAHV |
| 113 | LQEEDAGEYGCMWQEEM(O)ELYRQKV |
| 114 | LQVTDSGLYRCVIYHPPPLGEEM(O)RDRARAHVDALRTHLA |
| 115 | LQVTDSGLYRCVIYHPPPYLDDFQKKWQEEM(O)ELYRQKVE |
| 116 | [⁶⁴Cu]LQVTDSGLYRCVIYHPPPLGEEM(O)RDRARAHVDAL |
| | RTHLA |
| 117 | [⁶⁴Cu]LQVTDSGLYRCVIYHPPPYLDDFQKKWQEEM(O)ELY |
| | RQKVE |
| 118 | LQVTDSGLYRCVIYHPPGEEM(O)RDRARAHV |
| 119 | LQVTDSGLYRCVIYHPPWQEEM(O)ELYRQKV |
| 120 | MWKTPTLKYFPLGEEMRDRARAHVDALRTHLA |
| 121 | MWKTPTLKYFPYLDDFQKKWQEEMELYRQKVE |
| 122 | MWRTPTLRYFPLGEEMRDRARAHVDALRTHLA |
| 123 | MWRTPTLRYFPYLDDFQKKWQEEMELYRQKVE |
| 124 | [⁶⁴Cu]MWKTPTLKYFPLGEEMRDRARAHVDALRTHLA |
| 125 | [⁶⁴Cu]MWKTPTLKYFPYLDDFQKKWQEEMELYRQKVE |
| 126 | GARSMTLTVQARQLPLGEEMRDRARAHVDALRTHLA |
| 127 | GARSMTLTVQARQLPYLDDFQKKWQEEMELYRQKVE |
| 128 | [⁶⁴Cu]GARSMTLTVQARQLPLGEEMRDRARAHVDALRTHLA |
| 129 | [⁶⁴Cu]GARSMTLTVQARQLPYLDDFQKKWQEEMELYRQKVE |
| 130 | GVLRLLLFKLPLGEEMRDRARAHVDALRTHLA |
| 131 | GVLRLLLFKLPYLDDFQKKWQEEMELYRQKVE |
| 132 | [⁶⁴Cu]GVLRLLLFKLPLGEEMRDRARAHVDALRTHLA |
| 133 | [⁶⁴Cu]GVLRLLLFKLPYLDDFQKKWQEEMELYRQKVE |
| 134 | LGKATLYAVLPLGEEMRDRARAHVDALRTHLA |
| 135 | LGKATLYAVLPYLDDFQKKWQEEMELYRQKVE |
| 136 | [⁶⁴Cu]LGKATLYAVLPLGEEMRDRARAHVDALRTHLA |
| 137 | [⁶⁴Cu]LGKATLYAVLPYLDDFQKKWQEEMELYRQKVE |
| 138 | YLLDGILFIYPLGEEMRDRARAHVDALRTHLA |
| 139 | YLLDGILFIYPYLDDFQKKWQEEMELYRQKVE |
| 140 | [⁶⁴Cu]YLLDGILFIYPLGEEMRDRARAHVDALRTHLA |
| 141 | [⁶⁴Cu]YLLDGILFIYPYLDDFQKKWQEEMELYRQKVE |
| 142 | IIVTDVIATLPLGEEMRDRARAHVDALRTHLA |
| 143 | IIVTDVIATLPYLDDFQKKWQEEMELYRQKVE |
| 144 | [⁶⁴Cu]IIVTDVIATLPLGEEMRDRARAHVDALRTHLA |
| 145 | [⁶⁴Cu]IIVTDVIATLPYLDDFQKKWQEEMELYRQKVE |
| 146 | FLFAEIVSIFPLGEEMRDRARAHVDALRTHLA |
| 147 | FLFAEIVSIFPYLDDFQKKWQEEMELYRQKVE |
| 148 | [⁶⁴Cu]FLFAEIVSIFPLGEEMRDRARAHVDALRTHLA |
| 149 | [⁶⁴Cu]FLFAEIVSIFPYLDDFQKKWQEEMELYRQKVE |
| 150 | IVIVDICITGPLGEEMRDRARAHVDALRTHLA |
| 151 | IVIVDICITGPYLDDFQKKWQEEMELYRQKVE |
| 152 | [⁶⁴Cu]IVIVDICITGPLGEEMRDRARAHVDALRTHLA |
| 153 | [⁶⁴Cu]IVIVDICITGPYLDDFQKKWQEEMELYRQKVE |
| 154 | IAVAMGIRFIIMVAPLGEEMRDRARAHVDALRTHLA |
| 155 | IAVAMGIRFIIMVAPYLDDFQKKWQEEMELYRQKVE |
| 156 | GNLVRICLGAPLGEEMRDRARAHVDALRTHLA |
| 157 | GNLVRICLGAPYLDDFQKKWQEEMELYRQKVE |
| 158 | [⁶⁴Cu]GNLVRICLGAPLGEEMRDRARAHVDALRTHLA |
| 159 | [⁶⁴Cu]GNLVRICLGAPYLDDFQKKWQEEMELYRQKVE |
| 160 | VMGDLVLTVLPLGEEMRDRARAHVDALRTHLA |
| 161 | VMGDLVLTVLPYLDDFQKKWQEEMELYRQKVE |
| 162 | [⁶⁴Cu]VMGDLVLTVLPLGEEMRDRARAHVDALRTHLA |
| 163 | [⁶⁴Cu]VMGDLVLTVLPYLDDFQKKWQEEMELYRQKVE |
| 164 | LVAADAVASLPLGEEMRDRARAHVDALRTHLA |
| 165 | LVAADAVASLPYLDDFQKKWQEEMELYRQKVE |
| 166 | [⁶⁴Cu]LVAADAVASLPLGEEMRDRARAHVDALRTHLA |
| 167 | [⁶⁴Cu]LVAADAVASLPYLDDFQKKWQEEMELYRQKVE |
| 168 | SIATGMVGALLLLLVVALGIGLFMRPLGEEMRDRARAHVDAL |
| | RTHLA |
| 169 | SIATGMVGALLLLLVVALGIGLFMRPYLDDFQKKWQEEMELY |
| | RQKVE |
| 170 | DIVKLTVYDCIRRRRRRRRRPLGEEMRDRARAHVDALRTHLA |
| 171 | DIVKLTVYDCIRRRRRRRRRPYLDDFQKKWQEEMELYRQKVE |
| 172 | SLRRSSCFGGRMDRIGAQSGLGCNSFRYPLGEEMRDRARAHV |
| | DALRTHLA |
| 173 | SLRRSSCFGGRMDRIGAQSGLGCNSFRYPYLDDFQKKWQEEM |
| | ELYRQKVE |
| 174 | PtxGFLSKSLVFPLGEEMRDRARAHVDALRTHLA |
| 175 | PtxGFLSKSLVFPYLDDFQKKWQEEMELYRQKVE |
| 176 | PtxGFLSKSLVFPLGEEM(O)RDRARAHVDALRTHLA |
| 177 | PtxGFLSKSLVFPYLDDFQKKWQEEM(O)ELYRQKVE |
| 178 | IVILLAGGFLSKSLVFSVLFAPLGEEMRDRARAHVDALRTHL |
| | A |
| 179 | IVILLAGGFLSKSLVFSVLFAPYLDDFQKKWQEEMELYRQKV |
| | E |
| 180 | IVILLAGGFLSKSLVFSVLFA (parent) (TREM-1 TM |
| | peptide) |
| 181 | GFLSKSLVF (TREM-1 TM core peptide) |
| 182 | IVILLAGGFLSKSLVFSVLFA |
| 183 | IVILLAGGFLSKSLVFSVLFA |
| 184 | GSVILLAGGFLSKSLVFSVLFA |
| 185 | IVILLAGGFLSKSLVFSVLFA |
| 186 | KKILLAGGFLSKSLVFSVLFAKR |
| 187 | KKILLAGGFLSKSLVFSVLFAKR |
| 188 | (IVILLAGGFLSKSLVFSVLFA)₂^{c} |
| 189 | IVILLACGFLSKSLVFSVLFA |
| 190 | (IVILLAC*GFLSKSLVFSVLFA)₂^{d} |
| 191 | IVILLAGGFLSKSLVRSVLFA |
| 192 | IVILLAGGFLSKSLVRSVLFA |
| 193 | IVILLAGGFLSKSLVRSVLFA |
| 194 | GSILLAGGFLSKSLVRSVLFA |
| 195 | KKILLAGGFLSKSLVRSVLFAKR |
| 196 | KKILLAGGFLSKSLVRSVLFAKR |
| 197 | KKILLAGGFLSKSLVRSVLFAKR |
| 198 | (IVILLAGGFLSKSLVRSVLFA)₂ |
| 199 | IVILLACGFLSKSLVRSVLFA |
| 200 | (IVILLAC*GFLSKSLVRSVLFA)₂ |
| 201 | IVILLAGRFLSKSLVRSVLFA |
| 202 | IVILLAGRFLSKSLVRSVLFA |
| 203 | IVILLAGRFLSKSLVRSVLFA |
| 204 | KKILLAGRFLSKSLVRSVLFAKR |
| 205 | KKILLAGRFLSKSLVRSVLFAKR |
| 206 | KKILLAGRFLSKSLVRSVLFAKR |
| 207 | (IVILLAGRFLSKSLVRSVLFA)₂ |
| 208 | IVILLACRFLSKSLVRSVLFA |
| 209 | (IVILLAC*RFLSKSLVRSVLFA)₂ |
| 210 | GFLSKSLVF |
| 211 | GFLSKSLVF |
| 212 | GFLSKSLVF |
| 213 | (GFLSKSLVF)₂ |
| 214 | ACGFLSKSLVF |
| 215 | (AC*GFLSKSLVF)₂ |
| 216 | GLLSKSLVF |
| 217 | GLLSKSLVF |
| 218 | GLLSKSLVF |
| 219 | (GLLSKSLVF)₂ |
| 220 | ACGLLSKSLVF |
| 221 | (AC*GLLSKSLVF)₂ |
| 222 | GLLSKTLVF |
| 223 | GLLSKTLVF |
| 224 | GLLSKTLVF |
| 225 | (GLLSKTLVF)₂ |
| 226 | ACGLLSKTLVF |
| 227 | (AC*GLLSKTLVF)₂ |
| 228 | GFLSKSLVR |
| 229 | GFLSKSLVR |
| 230 | GFLSKSLVR |
| 231 | (GFLSKSLVR)₂ |
| 232 | ACGFLSKSLVR |
| 233 | (AC*GFLSKSLVR)₂ |
| 234 | KFLSKSLVR |
| 235 | KFLSKSLVR |
| 236 | KFLSKSLVR |
| 237 | (KFLSKSLVR)₂ |
| 238 | ACKFLSKSLVR |
| 239 | (AC*RFLSKSLVR)₂ |
| 240 | VTISVICGLLSKSLVFIILFI |
| 241 | (VTISVICGLLSKSLVFIILFI)₂ |
| 242 | (VTISVIC*GLLSKSLVFIILFI)₂ |
| 243 | IIIPAACGLLSKTLVFIGLFA |
| 244 | (IIIPAACGLLSKTLVFIGLFA)₂ |
| 245 | (IIIPAAC*GLLSKTLVFIGLFA)₂ |
| 246 | ILPAVCGLLSKSLVFIVLFVV |
| 247 | ILPAVCKLLSKSLVFIVLFVV |
| 248 | (ILPAVCGLLSKSLVFIVLFVV)₂ |
| 249 | (ILPAVC*GLLSKSLVFIVLFVV)₂ |

### INCORPORATION BY REFERENCE

All of the patents and publications cited herein are hereby incorporated by reference in their entirety. Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

U.S. PATENT DOCUMENTS, each of which is herein incorporated by reference in it's entirety:
1. Broersma, et al., U.S. Pat. No. 5,681,925, Oct. 28, 1997
2. Castaigne, et al., U.S. Pat. No. 9,161,988, Oct. 20, 2015
3. Castaigne, et al., U.S. Pat. No. 8,921,314, Dec. 30, 2014
4. Castaigne, et al., U.S. Pat. No. 9,173,891, Nov. 3, 2015
5. Caulfield, U.S. Pat. No. 8,114,613, Feb. 14, 2012
6. Eggink and Hoober, U.S. Pat. No. 7,811,995, Oct. 12, 2010
7. Eggink and Hoober, U.S. Pat. No. 8,496,942, Jul. 30, 2013
8. Elmore, U.S. Pat. No. 4,749,742, Jun. 7, 1988
9. Faure, et al., U.S. Pat. No. 8,013,116, Sep. 6, 2011
10. Faure, et al., U.S. Pat. No. 9,273,111, Mar. 1, 2016
11. Gibot, et al., U.S. Pat. No. 9,657,081, May 23, 2017
12. Gibot and Derive, U.S. Pat. No. 9,815,883, Nov. 14, 2017
13. Gibot and Derive, U.S. Pat. No. 9,255,136, Feb. 9, 2016
14. Hazen, et al., U.S. Pat. No. 8,338,110, Dec. 25, 2012
15. Kuai, et al. US2008/0247955. 2008.
16. Low and Turk, U.S. Pat. No. 8,916,167, Dec. 23, 2014
17. Manolios, U.S. Pat. No. 6,057,294, May 2, 2000
18. Manolios, U.S. Pat. No. 7,192,928, Mar. 20, 2007
19. Manolios, et al. US 20120077732. 2012
20. Manolios. US 20100267651. 2010
21. Morgan and Pandha. US 2012/0177672 A1. 2012
22. Schiffman and Altman, U.S. Pat. No. 4,427,660, Jan. 24, 1984
23. Sigalov, U.S. Pat. No. 8,278,271, Oct. 2, 2012
24. Sigalov, U.S. Pat. No. 8,614,188, Dec. 24, 2013
25. Sigalov. US 20130039948. 2013.
26. Sigalov, U.S. Pat. No. 9,981,004, May 29, 2018
27. Sigalov, U.S. Pat. No. 8,513,185, Aug. 20, 2013
28. Sigalov. US 20110256224. 2011.
29. Sigalov. US 20130045161. 2013.
30. Urso, et al., U.S. Pat. No. 9,127,064, Sep. 8, 2015
31. Wu and Chi, U.S. Pat. No. 9,387,257, Jul. 12, 2016
32. Wu, et al., U.S. Pat. No. 8,415,453, Apr. 9, 2013

### U.S. PATENT DOCUMENTS

US 20110256224
US 20130039948
US 20130045161
US 20140275026
US 20150005355
US 20150232531
US 20150306085
US 20160015773
US 20160193288
US 20190117725
U.S. Pat. No. 4,427,660
U.S. Pat. No. 4,749,742
U.S. Pat. No. 8,013,836
U.S. Pat. No. 8,022,047
U.S. Pat. No. 8,114,613
U.S. Pat. No. 8,338,110
U.S. Pat. No. 8,415,453
U.S. Pat. No. 8,513,185
U.S. Pat. No. 8,680,139
U.S. Pat. No. 8,916,167
U.S. Pat. No. 8,921,314
U.S. Pat. No. 9,000,127
U.S. Pat. No. 9,161,988
U.S. Pat. No. 9,173,891
U.S. Pat. No. 9,255,136
U.S. Pat. No. 9,273,111
U.S. Pat. No. 9,320,811
U.S. Pat. No. 9,550,830
U.S. Pat. No. 9,657,081
U.S. Pat. No. 9,717,717
U.S. Pat. No. 9,815,883
U.S. Pat. No. 9,981,004
   FOREIGN PATENT DOCUMENTS, each of which is herein incorporated by reference in it's entirety:
James and Andreasson, WO 2017083682A1, Publication Date: May 18, 2017,
   ADDITIONAL PUBLICATIONS, each of which is herein incorporated by reference in it's entirety:
Baghdadi et al. Sci Rep 2018,8:418.
Boussios et al. Ann Gastroenterol 2012,25:106-18.
Brynjolfsson et al. Inflamm Bowel Dis 2016,22:1803-11.
Clark et al. Cancer Res 2007,67:9518-27.
Colonna et al. J Infect Dis 2003,187 Suppl 2:S397-401.
Cuvier et al. Br J Clin Pharmacol 2018,84:2270-79.
Derive et al. J Immunol 2012,188:5585-92.
Dower et al. J Immunol 2008,180:3520-34.
Elgert et al. J Leukoc Biol 1998,64:275-90.
Esposito et al. J Clin Pathol 2004,57:630-6.
Francois et al. Intensive Care Med Exp 2018,6(Suppl 1).
Fung et al. Front Physiol 2017,8:841.
Gardian et al. Gastroenterol Res Pract 2012,2012:585674.
Ghoncheh et al. Asian Pac J Cancer Prev 2016,17:43-6.
Gibot et al. Infect Immun 2006,74:2823-30.
Gibot et al. J Exp Med 2004,200:1419-26.
Grivennikov et al. Cell 2010,140:883-99.
Groblewska et al. Clin Chem Lab Med 2007,45:30-4.
Grossman et al. Clin Cancer Res 2010,16:2443-9.
Ho et al. Am J Respir Crit Care Med 2008,177:763-70.
Ilic et al. World J Gastroenterol 2016,22:9694-705.
Irby et al. Mol Pharm 2017,14:1325-38.
Johnson et al. Neuro Oncol 2017,19:vi249.
Kubota et al. Journal of Experimental Medicine 2009,206:1089-102.
Kubota et al. J Exp Med 2009,206:1089-102.
Kuemmel et al. Sci Rep 2018,8:10766.
Lanier Immunol Rev 2009,227:150-60.
Laoui et al. Front Immunol 2014,5:489.
Lau et al. Bioorg Med Chem 2018,26:2700-07.
Liadaki et al. J Biol Chem 2000,275:21262-71.
Lin et al. Chem Commun (Camb) 2013,49:4968-70.
Liu et al. Nat Immunol 2019.
Liu et al. J Biol Chem 2002,277:21576-84.
Mantovani et al. Nat Rev Clin Oncol 2017,14:399-416.
Mroczko et al. Clin Chim Acta 2007,380:208-12.
Nguyen et al. Expert Rev Clin Immunol 2015,11:849-57.
Ohno et al. Mol Cancer Ther 2006,5:2634-43.
Page et al. (2009). Principles of chemotherapy. Cancer Management: A Multidisciplinary Approach. R. Pazdur, L. D. Wagman and K. A. Camphausen. Manhasset, N.Y., Cmp United Business Media: 21-37.
Partridge et al. Clin Cancer Res 2017,23:2611-16.
Paulus et al. Cancer Res 2006,66:4349-56.
Pei et al. J Thorac Cardiovasc Surg 2014,148:1208-16 e2.
Pelham et al. Expert Opin Ther Pat 2014,24:1383-95.
Prager et al. ESMO Open 2018,3:e000285.
Richardsen et al. Anticancer Res 2015,35:865-74.
Rojas et al. Biochim Biophys Acta 2018,1864:2761-68.
Rossi et al. Clin Cancer Res 2015,21:1248-57.
Schenk et al. J Clin Invest 2007,117:3097-106.
Shen et al. Mol Pharm 2017,14:4572-82.
Shen et al. J Cell Mol Med 2017,21:2524-34.
Shen et al. PLoS One 2015,10:e0143453.
Shergalis et al. Pharmacol Rev 2018,70:412-45.
Shih et al. J Cancer Mol 2006,2:101-06.
Sigalov Trends Immunol 2004,25:583-9.
Sigalov Trends Pharmacol Sci 2006,27:518-24.
Sigalov Self Nonself 2010,1:192-224.
Sigalov Contrast Media Mol Imaging 2014,9:372-82.
Sigalov Int Immunopharmacol 2014,21:208-19.
Sigalov Int Immunopharmacol 2014,21:208-19.
Sigalov Adv Protein Chem Struct Biol 2018,111:61-99.
Sigalov et al. Biochemistry 2004,43:2049-61.
Sigalov et al. FEBS Lett 1998,433:196-200.
Sigalov et al. Chem Phys Lipids 2001,113:133-46.
Skrzypski et al. Clin Lung Cancer 2013,14:666-73 e2.
Solinas et al. J Leukoc Biol 2009,86:1065-73.
Strojnik et al. Anticancer Res 2009,29:3269-79.
Stukas et al. J Am Heart Assoc 2014,3:e001156.
Tammaro et al. Pharmacol Ther 2017,177:81-95.
Tap et al. N Engl J Med 2015,373:428-37.
Tjomsland et al. Neoplasia 2011,13:664-75.
Tornai et al. Hepatol Commun 2019,3:99-115.
Tsukamoto et al. Cancer Res 2018,78:5011-22.
Turnbull et al. J Exp Med 2005,202:363-9.
Van Cutsem et al. Eur J Cancer 2013,49:2476-85.
Vlieghe et al. Drug Discov Today 2010,15:40-56.
Walker et al. World J Gastroenterol 2014,20:2224-36.
Weber et al. PLoS Pathog 2014,10:e1003900.
Welsh et al. J Clin Oncol 2005,23:8959-67.
Wong et al. Sci Rep 2018,8:1129.
Wong et al. Sci Rep 2017,7:45846.
Wu et al. Cancer Res 2012,72:3977-86.
Wu et al. Hepatology 2019.
Wu et al. Hepatology 2019,70:198-214.
Yako et al. PLoS One 2016,11:e0154016.
Yoshimura Cell Mol Immunol 2018,15:335-45.
Yuan et al. PLoS One 2014,9:e94241.
Yusen et al. J BUON 2018,23:398-402.
Zappa et al. Transl Lung Cancer Res 2016,5:288-300.
Zhou et al. Int Immunopharmacol 2013,17:155-61.
Zhu et al. Cancer Res 2014,74:5057-69.
1. Banga. Therapeutic peptides and proteins: formulation, processing, and delivery systems.
2nd ed. Boca Raton, Fla.: Taylor & Francis Group; 2006.
2. Bennasroune, et al., Transmembrane peptides as inhibitors of ErbB receptor signaling,
Mol Biol Cell 2004, 15:3464-3474.
3. Chang, et al., A novel peptide enhances therapeutic efficacy of liposomal anti-cancer drugs in mice models of human lung cancer, PLoS One 2009, 4:e4171.
4. Cunningham et al., Cell Permeant Polyphosphoinositide-binding Peptides That Block 5. Cell Motility and Actin Assembly, J Biol Chem 2001, 278: 43390-43399
6. Connor, et al., Quantification of oxygen-induced retinopathy in the mouse: a model of vessel loss, vessel regrowth and pathological angiogenesis, Nat Protoc 2009, 4:1565-1573.
7. Cuvier et al. Br J Clin Pharmacol 2018,84:2270-79.
8. Dutting, et al., Platelet GPVI: a target for antithrombotic therapy?!, Trends Pharmacol Sci 2012, 33:583-590.
9. Fung, et al., SR-BI Mediated Transcytosis of HDL in Brain Microvascular Endothelial Cells Is Independent of Caveolin, Clathrin, and PDZK1, Front Physiol 2017, 8:841.
10. Gibot, et al., Modulation of the triggering receptor expressed on the myeloid cell type 1 pathway in murine septic shock, Infect Immun 2006, 74:2823-2830.
11. Gibot, et al., Effects of the TREM 1 pathway modulation during hemorrhagic shock in rats, Shock 2009, 32:633-637.
12. Gibot, et al., TREM-1 promotes survival during septic shock in mice, Eur J Immunol 2007, 37:456-466.
13. Gonzalez, et al., NKG2D ligands: key targets of the immune response, Trends Immunol 2008, 29:397-403.
14. Irby, et al., Lipid-Drug Conjugate for Enhancing Drug Delivery, Mol Pharm 2017, 14:1325-1338.
15. Ito, et al., Blockade of NKG2D signaling prevents the development of murine CD4+ T cell-mediated colitis, Am J Physiol Gastrointest Liver Physiol 2008, 294:G199-207.
16. Joffre, et al., Genetic and Pharmacological Inhibition of TREM-1 Limits the Development of Experimental Atherosclerosis, J Am Coll Cardiol 2016, 68:2776-2793.
17. Johnson, et al., Relationships between drug activity in NCI preclinical in vitro and in vivo models and early clinical trials, Br J Cancer 2001, 84:1424-1431.
18. Johnson, et al., TMIC-30. TREM1-TARGETED PET IMAGING OF TUMOR-ASSOCIATED MACROPHAGES IN AN ORTHOTOPIC GLIOBLASTOMA MOUSE MODEL Neuro Oncol 2017, 19:vi249.
19. Jones, et al., Computer programs to identify and classify amphipathic alpha helical domains. J Lipid Res 1992, 33, 287-296.
20. Khan, et al., Inhibition of septic shock in mice by an oligopeptide from the beta-chain of human chorionic gonadotrophin hormone, Hum Immunol 2002, 63:1-7.
21. Kokten, et al., TREM-1 Inhibition Restores Impaired Autophagy Activity and Reduces Colitis in Mice, J Crohns Colitis 2018, 12:230-244.
22. Ladetzki-Baehs, et al., Atrial natriuretic peptide, a regulator of nuclear factor-kappaB activation in vivo, Endocrinology 2007, 148:332-336.
23. Lanier, DAP10- and DAP12-associated receptors in innate immunity, Immunol Rev 2009, 227:150-160.
24. Lin, et al., Supramolecular filaments containing a fixed 41% paclitaxel loading, Chem Commun (Camb) 2013, 49:4968-4970.
25. Liu, et al., The effects of mutations in helices 4 and 6 of ApoA-I on scavenger receptor class B type I (SR-B1)-mediated cholesterol efflux suggest that formation of a productive complex between reconstituted high density lipoprotein and SR-BI is required for efficient lipid transport, J Biol Chem 2002, 277:21576-21584.
26. Lopez-Soto, et al., NKG2D signaling in cancer immunosurveillance, Int J Cancer 2015, 136:1741-1750.
27. Manolios, et al., T-cell antigen receptor (TCR) transmembrane peptides: A new paradigm for the treatment of autoimmune diseases, Cell Adh Migr 2010, 4:273-283.
28. Overholser, et al., Epidermal growth factor receptor blockade by antibody IMC-C225 inhibits growth of a human pancreatic carcinoma xenograft in nude mice, Cancer 2000, 89:74-82.
29. Page and Takimoto. Principles of chemotherapy. In: Pazdur R, Wagman L D, Camphausen K A, editors. Cancer Management: A Multidisciplinary Approach. 11th ed. Manhasset, N.Y.: Cmp United Business Media; 2009. p. 21-37.
30. Patel, et al., Activation of the endothelin system mediates pathological angiogenesis during ischemic retinopathy, Am J Pathol 2014, 184:3040-3051.
31. Petrasek, et al., IL-1 receptor antagonist ameliorates inflammasome-dependent alcoholic steatohepatitis in mice, J Clin Invest 2012, 122:3476-3489.
32. Rojas, et al., Blockade of TREM-1 prevents vitreoretinal neovascularization in mice with oxygen-induced retinopathy, Biochim Biophys Acta 2018, 1864:2761-2768.
33. Saadipour, TREM1: A Potential Therapeutic Target For Alzheimer's Disease, Neurotox Res 2017, 32:14-16.
34. Schenk, et al., TREM-1-expressing intestinal macrophages crucially amplify chronic inflammation in experimental colitis and inflammatory bowel diseases, J Clin Invest 2007, 117:3097-3106.
35. Shen and Sigalov, Novel TREM-1 Inhibitors Attenuate Tumor Growth and Prolong Survival in Experimental Pancreatic Cancer, Mol Pharm 2017, 14:4572-4582.
36. Shen and Sigalov, Rationally designed ligand-independent peptide inhibitors of TREM-1 ameliorate collagen-induced arthritis, J Cell Mol Med 2017, 21:2524-2534.
37. Shen and Sigalov, SARS Coronavirus Fusion Peptide-Derived Sequence Suppresses Collagen-Induced Arthritis in DBA/1J Mice, Sci Rep 2016, 6:28672.
38. Shen, et al., Diagnostic Magnetic Resonance Imaging of Atherosclerosis in Apolipoprotein E Knockout Mouse Model Using Macrophage-Targeted Gadolinium-Containing Synthetic Lipopeptide Nanoparticles, PLoS One 2015, 10:e0143453.
39. Sigalov et al. Large-scale isolation and purification of human apolipoproteins A-I and A-II, J Chromatogr 1991, 537:464-468
40. Sigalov, Immune cell signaling: a novel mechanistic model reveals new therapeutic targets, Trends Pharmacol Sci 2006, 27:518-524.
41. Sigalov, More on: glycoprotein VI oligomerization: a novel concept of platelet inhibition, J Thromb Haemost 2007, 5:2310-2312.
42. Sigalov, Multichain immune recognition receptor signaling: different players, same game?, Trends Immunol 2004, 25:583-589.
43. Sigalov, Nature-inspired nanoformulations for contrast-enhanced in vivo MR imaging of macrophages, Contrast Media Mol Imaging 2014, 9:372-382.
44. Sigalov, A novel ligand-independent peptide inhibitor of TREM-1 suppresses tumor growth in human lung cancer xenografts and prolongs survival of mice with lipopolysaccharide-induced septic shock, Int Immunopharmacol 2014, 21:208-219.
45. Sigalov, Novel mechanistic concept of platelet inhibition, Expert Opin Ther Targets 2008, 12:677-692.
46. Sigalov, Novel mechanistic insights into viral modulation of immune receptor signaling, PLoS Pathog 2009, 5:e1000404.
47. Sigalov, The SCHOOL of nature: I. Transmembrane signaling, Self Nonself 2010, 1:4-39.
48. Sigalov, The SCHOOL of nature: III. From mechanistic understanding to novel therapies, Self Nonself 2010, 1:192-224.
49. Sigalov, Targeting Intramembrane Protein-Protein Interactions: Novel Therapeutic Strategy of Millions Years Old, Adv Protein Chem Struct Biol 2018, 111:61-99.
50. Sigalov and Stern, Enzymatic repair of oxidative damage to human apolipoprotein A-I, FEBS Lett 1998, 433:196-200.
51. Sigalov and Stern, Oxidation of methionine residues affects the structure and stability of apolipoprotein A-I in reconstituted high density lipoprotein particles, Chem Phys Lipids 2001, 113:133-146.
52. Sipsas, et al., Therapy of Mucormycosis, J Fungi (Basel) 2018, 4
53. Stevenson, Advances in peptide pharmaceuticals, Curr Pharm Biotechnol 2009, 10:122-137.
54. Stukas, et al., Intravenously injected human apolipoprotein A-I rapidly enters the central nervous system via the choroid plexus, J Am Heart Assoc 2014, 3:e001156.
55. Tammaro, et al., TREM-1 and its potential ligands in non-infectious diseases: from biology to clinical perspectives, Pharmacol Ther 2017, 177:81-95.
56. Tjin Tham Sjin, et al., A 27-amino-acid synthetic peptide corresponding to the NH2-terminal zinc-binding domain of endostatin is responsible for its antitumor activity, Cancer Res 2005, 65:3656-3663.
57. Tornai et al. Hepatol Commun 2019,3:99-115.
58. Tsung, et al., A novel inhibitory peptide of Toll-like receptor signaling limits lipopolysaccharide-induced production of inflammatory mediators and enhances survival in mice, Shock 2007, 27:364-369.
59. Turner, et al. Peptide Conjugates of Oligonucleotide Analogs and siRNA for Gene Expression Modulation. In: Langel U, ed, editor. Handbook of Cell-Penetrating Peptides. 2nd edition ed. Boca Raton: CRC Press; 2007. p. 313-328.
60. Ungerer, et al., The GPVI-Fc fusion protein Revacept reduces thrombus formation and improves vascular dysfunction in atherosclerosis without any impact on bleeding times, PLoS One 2013, 8:e71193.
61. Van Belle, et al., NKG2D blockade facilitates diabetes prevention by antigen-specific Tregs in a virus-induced model of diabetes, J Autoimmun 2013, 40:66-73.
62. Vilarinho, et al., Blockade of NKG2D on NKT cells prevents hepatitis and the acute immune response to hepatitis B virus, Proc Natl Acad Sci USA 2007, 104:18187-18192.
63. Vlieghe, et al., Synthetic therapeutic peptides: science and market, Drug Discov Today 2010, 15:40-56.
64. Zhou, et al., TREM-1 inhibition attenuates inflammation and tumor within the colon, Int Immunopharmacol 2013, 17:155-161.
1. Banga. Therapeutic peptides and proteins: formulation, processing, and delivery systems.
2nd ed. Boca Raton, Fla.: Taylor & Francis Group; 2006.
2. Bennasroune, et al., Transmembrane peptides as inhibitors of ErbB receptor signaling, Mol Biol Cell 2004, 15:3464-3474.
3. Chang, et al., A novel peptide enhances therapeutic efficacy of liposomal anti-cancer drugs in mice models of human lung cancer, PLoS One 2009, 4:e4171.
4. Connor, et al., Quantification of oxygen-induced retinopathy in the mouse: a model of vessel loss, vessel regrowth and pathological angiogenesis, Nat Protoc 2009, 4:1565-1573.
5. Cuvier, et al., A first-in-man safety and pharmacokinetics study of nangibotide, a new modulator of innate immune response through TREM-1 receptor inhibition, Br J Clin Pharmacol 2018, in press
6. Dutting, et al., Platelet GPVI: a target for antithrombotic therapy?!, Trends Pharmacol Sci 2012, 33:583-590.
7. Fung, et al., SR-BI Mediated Transcytosis of HDL in Brain Microvascular Endothelial Cells Is Independent of Caveolin, Clathrin, and PDZK1, Front Physiol 2017, 8:841.
8. Gibot, et al., Modulation of the triggering receptor expressed on the myeloid cell type 1 pathway in murine septic shock, Infect Immun 2006, 74:2823-2830.
9. Gibot, et al., Effects of the TREM 1 pathway modulation during hemorrhagic shock in rats, Shock 2009, 32:633-637.
10. Gibot, et al., TREM-1 promotes survival during septic shock in mice, Eur J Immunol 2007, 37:456-466.
11. Gonzalez, et al., NKG2D ligands: key targets of the immune response, Trends Immunol 2008, 29:397-403.
12. Irby, et al., Lipid-Drug Conjugate for Enhancing Drug Delivery, Mol Pharm 2017, 14:1325-1338.
13. Ito, et al., Blockade of NKG2D signaling prevents the development of murine CD4+ T cell-mediated colitis, Am J Physiol Gastrointest Liver Physiol 2008, 294:G199-207.
14. Joffre, et al., Genetic and Pharmacological Inhibition of TREM-1 Limits the Development of Experimental Atherosclerosis, J Am Coll Cardiol 2016, 68:2776-2793.
15. Johnson, et al., Relationships between drug activity in NCI preclinical in vitro and in vivo models and early clinical trials, Br J Cancer 2001, 84:1424-1431.
16. Johnson, et al., TMIC-30. TREM1-TARGETED PET IMAGING OF TUMOR-ASSOCIATED MACROPHAGES IN AN ORTHOTOPIC GLIOBLASTOMA MOUSE MODEL Neuro Oncol 2017, 19:vi249.
17. Khan, et al., Inhibition of septic shock in mice by an oligopeptide from the beta-chain of human chorionic gonadotrophin hormone, Hum Immunol 2002, 63:1-7.
18. Kokten, et al., TREM-1 Inhibition Restores Impaired Autophagy Activity and Reduces Colitis in Mice, J Crohns Colitis 2018, 12:230-244.
19. Ladetzki-Baehs, et al., Atrial natriuretic peptide, a regulator of nuclear factor-kappaB activation in vivo, Endocrinology 2007, 148:332-336.
20. Lanier, DAP10- and DAP12-associated receptors in innate immunity, Immunol Rev 2009, 227:150-160.
21. Lin, et al., Supramolecular filaments containing a fixed 41% paclitaxel loading, Chem Commun (Camb) 2013, 49:4968-4970.
22. Liu, et al., The effects of mutations in helices 4 and 6 of ApoA-I on scavenger receptor class B type I (SR-BI)-mediated cholesterol efflux suggest that formation of a productive complex between reconstituted high density lipoprotein and SR-BI is required for efficient lipid transport, J Biol Chem 2002, 277:21576-21584.
23. Lopez-Soto, et al., NKG2D signaling in cancer immunosurveillance, Int J Cancer 2015, 136:1741-1750.
24. Manolios, et al., T-cell antigen receptor (TCR) transmembrane peptides: A new paradigm for the treatment of autoimmune diseases, Cell Adh Migr 2010, 4:273-283.
25. Overholser, et al., Epidermal growth factor receptor blockade by antibody IMC-C225 inhibits growth of a human pancreatic carcinoma xenograft in nude mice, Cancer 2000, 89:74-82.
26. Page and Takimoto. Principles of chemotherapy. In: Pazdur R, Wagman L D, Camphausen K A, editors. Cancer Management: A Multidisciplinary Approach. 11th ed. Manhasset, N.Y.: Cmp United Business Media; 2009. p. 21-37.
27. Patel, et al., Activation of the endothelin system mediates pathological angiogenesis during ischemic retinopathy, Am J Pathol 2014, 184:3040-3051.
28. Petrasek, et al., IL-1 receptor antagonist ameliorates inflammasome-dependent alcoholic steatohepatitis in mice, J Clin Invest 2012, 122:3476-3489.
29. Rojas, et al., Blockade of TREM-1 prevents vitreoretinal neovascularization in mice with oxygen-induced retinopathy, Biochim Biophys Acta 2018, 1864:2761-2768.
30. Saadipour, TREM1: A Potential Therapeutic Target For Alzheimer's Disease, Neurotox Res 2017, 32:14-16.
31. Schenk, et al., TREM-1-expressing intestinal macrophages crucially amplify chronic inflammation in experimental colitis and inflammatory bowel diseases, J Clin Invest 2007, 117:3097-3106.
32. Shen and Sigalov, Novel TREM-1 Inhibitors Attenuate Tumor Growth and Prolong Survival in Experimental Pancreatic Cancer, Mol Pharm 2017, 14:4572-4582.
33. Shen and Sigalov, Rationally designed ligand-independent peptide inhibitors of TREM-1 ameliorate collagen-induced arthritis, J Cell Mol Med 2017, 21:2524-2534.
34. Shen and Sigalov, SARS Coronavirus Fusion Peptide-Derived Sequence Suppresses Collagen-Induced Arthritis in DBA/1J Mice, Sci Rep 2016, 6:28672.
35. Shen, et al., Diagnostic Magnetic Resonance Imaging of Atherosclerosis in Apolipoprotein E Knockout Mouse Model Using Macrophage-Targeted Gadolinium-Containing Synthetic Lipopeptide Nanoparticles, PLoS One 2015, 10:e0143453.
36. Sigalov, Immune cell signaling: a novel mechanistic model reveals new therapeutic targets, Trends Pharmacol Sci 2006, 27:518-524.
37. Sigalov, More on: glycoprotein VI oligomerization: a novel concept of platelet inhibition, J Thromb Haemost 2007, 5:2310-2312.
38. Sigalov, Multichain immune recognition receptor signaling: different players, same game?, Trends Immunol 2004, 25:583-589.
39. Sigalov, Nature-inspired nanoformulations for contrast-enhanced in vivo MR imaging of macrophages, Contrast Media Mol Imaging 2014, 9:372-382.
40. Sigalov, A novel ligand-independent peptide inhibitor of TREM-1 suppresses tumor growth in human lung cancer xenografts and prolongs survival of mice with lipopolysaccharide-induced septic shock, Int Immunopharmacol 2014, 21:208-219.
41. Sigalov, Novel mechanistic concept of platelet inhibition, Expert Opin Ther Targets 2008, 12:677-692.
42. Sigalov, Novel mechanistic insights into viral modulation of immune receptor signaling, PLoS Pathog 2009, 5:e1000404.
43. Sigalov, The SCHOOL of nature: I. Transmembrane signaling, Self Nonself 2010, 1:4-39.
44. Sigalov, The SCHOOL of nature: III. From mechanistic understanding to novel therapies, Self Nonself 2010, 1:192-224.
45. Sigalov, Targeting Intramembrane Protein-Protein Interactions: Novel Therapeutic Strategy of Millions Years Old, Adv Protein Chem Struct Biol 2018, 111:61-99.
46. Sigalov and Stern, Enzymatic repair of oxidative damage to human apolipoprotein A-I, FEBS Lett 1998, 433:196-200.
47. Sigalov and Stern, Oxidation of methionine residues affects the structure and stability of apolipoprotein A-I in reconstituted high density lipoprotein particles, Chem Phys Lipids 2001, 113:133-146.
48. Sipsas, et al., Therapy of Mucormycosis, J Fungi (Basel) 2018, 4
49. Stevenson, Advances in peptide pharmaceuticals, Curr Pharm Biotechnol 2009, 10:122-137.
50. Stukas, et al., Intravenously injected human apolipoprotein A-I rapidly enters the central nervous system via the choroid plexus, J Am Heart Assoc 2014, 3:e001156.
51. Tammaro, et al., TREM-1 and its potential ligands in non-infectious diseases: from biology to clinical perspectives, Pharmacol Ther 2017, 177:81-95.
52. Tjin Tham Sjin, et al., A 27-amino-acid synthetic peptide corresponding to the NH2-terminal zinc-binding domain of endostatin is responsible for its antitumor activity, Cancer Res 2005, 65:3656-3663.
53. Tornai, et al., TREM-1 inhibition ameliorates inflammation and macrophage and neutrophil activation in alcoholic liver disease in mice, Hepatology Communications 2018, in press
54. Tsung, et al., A novel inhibitory peptide of Toll-like receptor signaling limits lipopolysaccharide-induced production of inflammatory mediators and enhances survival in mice, Shock 2007, 27:364-369.
55. Turner, et al. Peptide Conjugates of Oligonucleotide Analogs and siRNA for Gene Expression Modulation. In: Langel U, ed, editor. Handbook of Cell-Penetrating Peptides. 2nd edition ed. Boca Raton: CRC Press; 2007. p. 313-328.
56. Ungerer, et al., The GPVI-Fc fusion protein Revacept reduces thrombus formation and improves vascular dysfunction in atherosclerosis without any impact on bleeding times, PLoS One 2013, 8:e71193.
57. Van Belle, et al., NKG2D blockade facilitates diabetes prevention by antigen-specific Tregs in a virus-induced model of diabetes, J Autoimmun 2013, 40:66-73.
58. Vilarinho, et al., Blockade of NKG2D on NKT cells prevents hepatitis and the acute immune response to hepatitis B virus, Proc Natl Acad Sci USA 2007, 104:18187-18192.
59. Vlieghe, et al., Synthetic therapeutic peptides: science and market, Drug Discov Today 2010, 15:40-56.
60. Zhou, et al., TREM-1 inhibition attenuates inflammation and tumor within the colon, Int Immunopharmacol 2013, 17:155-161.
American Cancer Society: Cancer Facts and Figures 2010, American Cancer Society, Atlanta, Ga. 2010,
Bowman and Joyce, Therapeutic targeting of tumor-associated macrophages and microglia in glioblastoma, Immunotherapy 2014, 6:663-666.
Clark, et al., Dynamics of the immune reaction to pancreatic cancer from inception to invasion, Cancer Res 2007, 67:9518-9527.
Colonna and Facchetti, TREM-1 (triggering receptor expressed on myeloid cells): a new player in acute inflammatory responses, J Infect Dis 2003, 187 Suppl 2:S397-401.
Condeelis and Pollard, Macrophages: obligate partners for tumor cell migration, invasion, and metastasis, Cell 2006, 124:263-266.
Elgert, et al., Tumor-induced immune dysfunction: the macrophage connection, J Leukoc Biol 1998, 64:275-290.
Esposito, et al., Inflammatory cells contribute to the generation of an angiogenic phenotype in pancreatic ductal adenocarcinoma, J Clin Pathol 2004, 57:630-636.
Feurino, et al., Current update of cytokines in pancreatic cancer: pathogenic mechanisms, clinical indication, and therapeutic values, Cancer Invest 2006, 24:696-703.
Furman, et al., Studies of the Metabolism of Radioiodinated Human Serum Alpha Lipoprotein in Normal and Hyperlipidemic Subjects, J Lab Clin Med 1964, 63:193-204.
Gardian, et al., Analysis of Pancreatic Cancer Microenvironment: Role of Macrophage Infiltrates and Growth Factors Expression, J Cancer 2012, 3:285-291.
Groblewska, et al., Serum levels of granulocyte colony-stimulating factor (G-CSF) and macrophage colony-stimulating factor (M-CSF) in pancreatic cancer patients, Clin Chem Lab Med 2007, 45:30-34.
Gupta, et al., Oral delivery of therapeutic proteins and peptides: a review on recent developments, Drug Deliv 2013, 20:237-246.
Hariharan, et al., Analysis of mortality rates for pancreatic cancer across the world, HPB (Oxford) 2008, 10:58-62.
Ho, et al., TREM-1 expression in tumor-associated macrophages and clinical outcome in lung cancer, Am J Respir Crit Care Med 2008, 177:763-770.
Jinushi and Komohara, Tumor-associated macrophages as an emerging target against tumors: Creating a new path from bench to bedside, Biochim Biophys Acta 2015, 1855:123-130.
Julovi, et al., Apolipoprotein A-II Plus Lipid Emulsion Enhance Cell Growth via SR-B1 and Target Pancreatic Cancer In Vitro and In Vivo, PLoS One 2016, 11:e0151475.
Komohara, et al., Tumor-associated macrophages: Potential therapeutic targets for anti-cancer therapy, Adv Drug Deliv Rev 2016, 99:180-185.
Kubota, et al., M-CSF inhibition selectively targets pathological angiogenesis and lymphangiogenesis, J Exp Med 2009, 206:1089-1102.
Kumari, et al., Role of interleukin-6 in cancer progression and therapeutic resistance, Tumour Biol 2016, 37:11553-11572.
Kuwahara, et al., Expressions of angiogenic factors in pancreatic ductal carcinoma: a correlative study with clinicopathologic parameters and patient survival, Pancreas 2003, 26:344-349.
Lagler, et al., TREM-1 activation alters the dynamics of pulmonary IRAK-M expression in vivo and improves host defense during pneumococcal pneumonia, J Immunol 2009, 183:2027-2036.
Lewis and Pollard, Distinct role of macrophages in different tumor microenvironments, Cancer Res 2006, 66:605-612.
Lin, et al., Macrophages regulate the angiogenic switch in a mouse model of breast cancer, Cancer Res 2006, 66:11238-11246.
Lin, et al., Colony-stimulating factor 1 promotes progression of mammary tumors to malignancy, J Exp Med 2001, 193:727-740.
Liu, et al., The effects of mutations in helices 4 and 6 of ApoA-I on scavenger receptor class B type I (SR-BI)-mediated cholesterol efflux suggest that formation of a productive complex between reconstituted high density lipoprotein and SR-BI is required for efficient lipid transport, J Biol Chem 2002, 277:21576-21584.
Mitchem, et al., Targeting tumor-infiltrating macrophages decreases tumor-initiating cells, relieves immunosuppression, and improves chemotherapeutic responses, Cancer Res 2013, 73:1128-1141.
Pelham and Agrawal, Emerging roles for triggering receptor expressed on myeloid cells receptor family signaling in inflammatory diseases, Expert Rev Clin Immunol 2014, 10:243-256.
Pieramici and Rabena, Anti-VEGF therapy: comparison of current and future agents, Eye (Lond) 2008, 22:1330-1336.
Schenk, et al., TREM-1-expressing intestinal macrophages crucially amplify chronic inflammation in experimental colitis and inflammatory bowel diseases, J Clin Invest 2007, 117:3097-3106.
Schneider, et al., Pancreatic cancer: basic and clinical aspects, Gastroenterology 2005, 128:1606-1625.
Shen and Sigalov, Rationally designed ligand-independent peptide inhibitors of TREM-1 ameliorate collagen-induced arthritis, J Cell Mol Med 2017:in press.
Shen and Sigalov, SARS Coronavirus Fusion Peptide-Derived Sequence Suppresses Collagen-Induced Arthritis in DBA/1J Mice, Sci Rep 2016, 6:28672.
Shen, et al., Diagnostic Magnetic Resonance Imaging of Atherosclerosis in Apolipoprotein E Knockout Mouse Model Using Macrophage-Targeted Gadolinium-Containing Synthetic Lipopeptide Nanoparticles, PLoS One 2015, 10:e0143453.
Shih, et al., Tumor-Associated Macrophage: Its Role in Cancer Invasion and Metastasis, J Cancer Mol 2006, 2:101-106.
Sigalov, Nature-inspired nanoformulations for contrast-enhanced in vivo MR imaging of macrophages, Contrast Media Mol Imaging 2014, 9:372-382.
Sigalov, A novel ligand-independent peptide inhibitor of TREM-1 suppresses tumor growth in human lung cancer xenografts and prolongs survival of mice with lipopolysaccharide-induced septic shock, Int Immunopharmacol 2014, 21:208-219.
Sigalov, Novel mechanistic concept of platelet inhibition, Expert Opin Ther Targets 2008, 12:677-692.
Sigalov, Novel mechanistic insights into viral modulation of immune receptor signaling, PLoS Pathog 2009, 5:e1000404.
Solinas, et al., Tumor-associated macrophages (TAM) as major players of the cancer-related inflammation, J Leukoc Biol 2009, 86:1065-1073.
Tjomsland, et al., Interleukin 1alpha sustains the expression of inflammatory factors in human pancreatic cancer microenvironment by targeting cancer-associated fibroblasts, Neoplasia 2011, 13:664-675.
Varney, et al., Tumour-associated macrophage infiltration, neovascularization and aggressiveness in malignant melanoma: role of monocyte chemotactic protein-1 and vascular endothelial growth factor-A, Melanoma Res 2005, 15:417-425.
Walker and Ko, Beyond first-line chemotherapy for advanced pancreatic cancer: an expanding array of therapeutic options?, World J Gastroenterol 2014, 20:2224-2236.
Wang, et al., T cell antigen receptor (TCR) transmembrane peptides colocalize with TCR, not lipid rafts, in surface membranes, Cell Immunol 2002, 215:12-19.
Wang, et al., Expression of TREM-1 mRNA in acute pancreatitis, World J Gastroenterol 2004, 10:2744-2746.
Wang, et al., Immunohistochemistry in the evaluation of neovascularization in tumor xenografts, Biotech Histochem 2008, 83:179-189.
Weber, et al., TREM-1 Deficiency Can Attenuate Disease Severity without Affecting Pathogen Clearance, PLoS Pathog 2014, 10:e1003900.
Yako, et al., Cytokines as Biomarkers of Pancreatic Ductal Adenocarcinoma: A Systematic Review, PLoS One 2016, 11:e0154016.
Zhu, et al., CSF1/CSF1R Blockade Reprograms Tumor-Infiltrating Macrophages and Improves Response to T-cell Checkpoint Immunotherapy in Pancreatic Cancer Models, Cancer Res 2014, 74:5057-5069.
Zumsteg, et al., Myeloid cells contribute to tumor lymphangiogenesis, PLoS One 2009, 4:e7067.

### U.S. PATENT DOCUMENTS

Broersma, et al., U.S. Pat. No. 5,681,925, Oct. 28, 1997
Castaigne, et al., U.S. Pat. No. 9,161,988, Oct. 20, 2015
Castaigne, et al., U.S. Pat. No. 8,921,314, Dec. 30, 2014
Castaigne, et al., U.S. Pat. No. 9,173,891, Nov. 3, 2015
Caulfield, U.S. Pat. No. 8,114,613, Feb. 14, 2012
Eggink and Hoober, U.S. Pat. No. 7,811,995, Oct. 12, 2010
Eggink and Hoober, U.S. Pat. No. 8,496,942, Jul. 30, 2013
Elmore, U.S. Pat. No. 4,749,742, Jun. 7, 1988
Faure, et al., U.S. Pat. No. 8,013,116, Sep. 6, 2011
Faure, et al., U.S. Pat. No. 9,273,111, Mar. 1, 2016
Gibot, et al., U.S. Pat. No. 9,657,081, May 23, 2017
Gibot and Derive, U.S. Pat. No. 9,815,883, Nov. 14, 2017
Gibot and Derive, U.S. Pat. No. 9,255,136, Feb. 9, 2016
Hazen, et al., U.S. Pat. No. 8,338,110, Dec. 25, 2012
Kuai, et al. US2008/0247955. 2008.
Low and Turk, U.S. Pat. No. 8,916,167, Dec. 23, 2014
Manolios, U.S. Pat. No. 6,057,294, May 2, 2000
Manolios, U.S. Pat. No. 7,192,928, Mar. 20, 2007
Manolios, et al. US 20120077732. 2012
Manolios. US 20100267651. 2010
Morgan and Pandha. US 2012/0177672 A1. 2012
Schiffman and Altman, U.S. Pat. No. 4,427,660, Jan. 24, 1984
Sigalov, U.S. Pat. No. 8,278,271, Oct. 2, 2012
Sigalov, U.S. Pat. No. 8,614,188, Dec. 24, 2013
Sigalov. US 20130039948. 2013.
Sigalov, U.S. Pat. No. 9,981,004, May 29, 2018
Sigalov, U.S. Pat. No. 8,513,185, Aug. 20, 2013
Sigalov. US 20110256224. 2011.
Sigalov. US 20130045161. 2013.
Urso, et al., U.S. Pat. No. 9,127,064, Sep. 8, 2015
Wu and Chi, U.S. Pat. No. 9,387,257, Jul. 12, 2016
Wu, et al., U.S. Pat. No. 8,415,453, Apr. 9, 2013

### OTHER REFERENCES

Banga. Therapeutic peptides and proteins: formulation, processing, and delivery systems. 2nd ed. Boca Raton, Fla.: Taylor & Francis Group; 2006.
Bennasroune, et al., Transmembrane peptides as inhibitors of ErbB receptor signaling, Mol Biol Cell 2004, 15:3464-3474.
Chang, et al., A novel peptide enhances therapeutic efficacy of liposomal anti-cancer drugs in mice models of human lung cancer, PLoS One 2009, 4:e4171.
Connor, et al., Quantification of oxygen-induced retinopathy in the mouse: a model of vessel loss, vessel regrowth and pathological angiogenesis, Nat Protoc 2009, 4:1565-1573.
Cuvier, et al., A first-in-man safety and pharmacokinetics study of nangibotide, a new modulator of innate immune response through TREM-1 receptor inhibition, Br J Clin Pharmacol 2018, in press
Dutting, et al., Platelet GPVI: a target for antithrombotic therapy?!, Trends Pharmacol Sci 2012, 33:583-590.
Fung, et al., SR-BI Mediated Transcytosis of HDL in Brain Microvascular Endothelial Cells Is Independent of Caveolin, Clathrin, and PDZK1, Front Physiol 2017, 8:841.
Gibot, et al., Modulation of the triggering receptor expressed on the myeloid cell type 1 pathway in murine septic shock, Infect Immun 2006, 74:2823-2830.
Gibot, et al., Effects of the TREM 1 pathway modulation during hemorrhagic shock in rats, Shock 2009, 32:633-637.
Gibot, et al., TREM-1 promotes survival during septic shock in mice, Eur J Immunol 2007, 37:456-466.
Gonzalez, et al., NKG2D ligands: key targets of the immune response, Trends Immunol 2008, 29:397-403.
Irby, et al., Lipid-Drug Conjugate for Enhancing Drug Delivery, Mol Pharm 2017, 14:1325-1338.
Ito, et al., Blockade of NKG2D signaling prevents the development of murine CD4+ T cell-mediated colitis, Am J Physiol Gastrointest Liver Physiol 2008, 294:G199-207.
Joffre, et al., Genetic and Pharmacological Inhibition of TREM-1 Limits the Development of Experimental Atherosclerosis, J Am Coll Cardiol 2016, 68:2776-2793.
Johnson, et al., Relationships between drug activity in NCI preclinical in vitro and in vivo models and early clinical trials, Br J Cancer 2001, 84:1424-1431.
Johnson, et al., TMIC-30. TREM1-TARGETED PET IMAGING OF TUMOR-ASSOCIATED MACROPHAGES IN AN ORTHOTOPIC GLIOBLASTOMA MOUSE MODEL Neuro Oncol 2017, 19:vi249.
Khan, et al., Inhibition of septic shock in mice by an oligopeptide from the beta-chain of human chorionic gonadotrophin hormone, Hum Immunol 2002, 63:1-7.
Kokten, et al., TREM-1 Inhibition Restores Impaired Autophagy Activity and Reduces Colitis in Mice, J Crohns Colitis 2018, 12:230-244.
Ladetzki-Baehs, et al., Atrial natriuretic peptide, a regulator of nuclear factor-kappaB activation in vivo, Endocrinology 2007, 148:332-336.
Lanier, DAP10- and DAP12-associated receptors in innate immunity, Immunol Rev 2009, 227:150-160.
Lin, et al., Supramolecular filaments containing a fixed 41% paclitaxel loading, Chem Commun (Camb) 2013, 49:4968-4970.
Liu, et al., The effects of mutations in helices 4 and 6 of ApoA-I on scavenger receptor class B type I (SR-BI)-mediated cholesterol efflux suggest that formation of a productive complex between reconstituted high density lipoprotein and SR-BI is required for efficient lipid transport, J Biol Chem 2002, 277:21576-21584.
Lopez-Soto, et al., NKG2D signaling in cancer immunosurveillance, Int J Cancer 2015, 136:1741-1750.
Manolios, et al., T-cell antigen receptor (TCR) transmembrane peptides: A new paradigm for the treatment of autoimmune diseases, Cell Adh Migr 2010, 4:273-283.
Overholser, et al., Epidermal growth factor receptor blockade by antibody IMC-C225 inhibits growth of a human pancreatic carcinoma xenograft in nude mice, Cancer 2000, 89:74-82.
Page and Takimoto. Principles of chemotherapy. In: Pazdur R, Wagman L D, Camphausen K A, editors. Cancer Management: A Multidisciplinary Approach. 11th ed. Manhasset, N.Y.: Cmp United Business Media; 2009. p. 21-37.
Patel, et al., Activation of the endothelin system mediates pathological angiogenesis during ischemic retinopathy, Am J Pathol 2014, 184:3040-3051.
Petrasek, et al., IL-1 receptor antagonist ameliorates inflammasome-dependent alcoholic steatohepatitis in mice, J Clin Invest 2012, 122:3476-3489.
Rojas, et al., Blockade of TREM-1 prevents vitreoretinal neovascularization in mice with oxygen-induced retinopathy, Biochim Biophys Acta 2018, 1864:2761-2768.
Saadipour, TREM1: A Potential Therapeutic Target For Alzheimer's Disease, Neurotox Res 2017, 32:14-16.
Schenk, et al., TREM-1-expressing intestinal macrophages crucially amplify chronic inflammation in experimental colitis and inflammatory bowel diseases, J Clin Invest 2007, 117:3097-3106.
Shen and Sigalov, Novel TREM-1 Inhibitors Attenuate Tumor Growth and Prolong Survival in Experimental Pancreatic Cancer, Mol Pharm 2017, 14:4572-4582.
Shen and Sigalov, Rationally designed ligand-independent peptide inhibitors of TREM-1 ameliorate collagen-induced arthritis, J Cell Mol Med 2017, 21:2524-2534.
Shen and Sigalov, SARS Coronavirus Fusion Peptide-Derived Sequence Suppresses Collagen-Induced Arthritis in DBA/1J Mice, Sci Rep 2016, 6:28672.
Shen, et al., Diagnostic Magnetic Resonance Imaging of Atherosclerosis in Apolipoprotein E Knockout Mouse Model Using Macrophage-Targeted Gadolinium-Containing Synthetic Lipopeptide Nanoparticles, PLoS One 2015, 10:e0143453.
Sigalov, Immune cell signaling: a novel mechanistic model reveals new therapeutic targets, Trends Pharmacol Sci 2006, 27:518-524.
Sigalov, More on: glycoprotein VI oligomerization: a novel concept of platelet inhibition, J Thromb Haemost 2007, 5:2310-2312.
Sigalov, Multichain immune recognition receptor signaling: different players, same game?, Trends Immunol 2004, 25:583-589.
Sigalov, Nature-inspired nanoformulations for contrast-enhanced in vivo MR imaging of macrophages, Contrast Media Mol Imaging 2014, 9:372-382.
Sigalov, A novel ligand-independent peptide inhibitor of TREM-1 suppresses tumor growth in human lung cancer xenografts and prolongs survival of mice with lipopolysaccharide-induced septic shock, Int Immunopharmacol 2014, 21:208-219.
Sigalov, Novel mechanistic concept of platelet inhibition, Expert Opin Ther Targets 2008, 12:677-692.
Sigalov, Novel mechanistic insights into viral modulation of immune receptor signaling, PLoS Pathog 2009, 5:e1000404.
Sigalov, The SCHOOL of nature: I. Transmembrane signaling, Self Nonself 2010, 1:4-39.
Sigalov, The SCHOOL of nature: III. From mechanistic understanding to novel therapies, Self Nonself 2010, 1:192-224.
Sigalov, Targeting Intramembrane Protein-Protein Interactions: Novel Therapeutic Strategy of Millions Years Old, Adv Protein Chem Struct Biol 2018, 111:61-99.
Sigalov and Stern, Enzymatic repair of oxidative damage to human apolipoprotein A-I, FEBS Lett 1998, 433:196-200.
Sigalov and Stem, Oxidation of methionine residues affects the structure and stability of apolipoprotein A-I in reconstituted high density lipoprotein particles, Chem Phys Lipids 2001, 113:133-146.
Sipsas, et al., Therapy of Mucormycosis, J Fungi (Basel) 2018, 4 Stevenson, Advances in peptide pharmaceuticals, Curr Pharm Biotechnol 2009, 10:122-137.
Stukas, et al., Intravenously injected human apolipoprotein A-I rapidly enters the central nervous system via the choroid plexus, J Am Heart Assoc 2014, 3:e001156.
Tammaro, et al., TREM-1 and its potential ligands in non-infectious diseases: from biology to clinical perspectives, Pharmacol Ther 2017, 177:81-95.
Tjin Tham Sjin, et al., A 27-amino-acid synthetic peptide corresponding to the NH2-terminal zinc-binding domain of endostatin is responsible for its antitumor activity, Cancer Res 2005, 65:3656-3663.
Tornai, et al., TREM-1 inhibition ameliorates inflammation and macrophage and neutrophil activation in alcoholic liver disease in mice, Hepatology Communications 2018, in press Tsung, et al., A novel inhibitory peptide of Toll-like receptor signaling limits lipopolysaccharide-induced production of inflammatory mediators and enhances survival in mice, Shock 2007, 27:364-369.
Turner, et al. Peptide Conjugates of Oligonucleotide Analogs and siRNA for Gene Expression Modulation. In: Langel U, ed, editor. Handbook of Cell-Penetrating Peptides. 2nd edition ed. Boca Raton: CRC Press; 2007. p. 313-328.
Ungerer, et al., The GPVI-Fc fusion protein Revacept reduces thrombus formation and improves vascular dysfunction in atherosclerosis without any impact on bleeding times, PLoS One 2013, 8:e71193.
Van Belle, et al., NKG2D blockade facilitates diabetes prevention by antigen-specific Tregs in a virus-induced model of diabetes, J Autoimmun 2013, 40:66-73.
Vilarinho, et al., Blockade of NKG2D on NKT cells prevents hepatitis and the acute immune response to hepatitis B virus, Proc Natl Acad Sci USA 2007, 104:18187-18192.
Vlieghe, et al., Synthetic therapeutic peptides: science and market, Drug Discov Today 2010, 15:40-56.
Zhou, et al., TREM-1 inhibition attenuates inflammation and tumor within the colon, Int Immunopharmacol 2013, 17:155-161.
American Cancer Society: Cancer Facts and Figures 2010, American Cancer Society, Atlanta, Ga. 2010,
Bowman and Joyce, Therapeutic targeting of tumor-associated macrophages and microglia in glioblastoma, Immunotherapy 2014, 6:663-666.
Clark, et al., Dynamics of the immune reaction to pancreatic cancer from inception to invasion, Cancer Res 2007, 67:9518-9527.
Colonna and Facchetti, TREM-1 (triggering receptor expressed on myeloid cells): a new player in acute inflammatory responses, J Infect Dis 2003, 187 Suppl 2:S397-401.
Condeelis and Pollard, Macrophages: obligate partners for tumor cell migration, invasion, and metastasis, Cell 2006, 124:263-266.
Elgert, et al., Tumor-induced immune dysfunction: the macrophage connection, J Leukoc Biol 1998, 64:275-290.
Esposito, et al., Inflammatory cells contribute to the generation of an angiogenic phenotype in pancreatic ductal adenocarcinoma, J Clin Pathol 2004, 57:630-636.
Feurino, et al., Current update of cytokines in pancreatic cancer: pathogenic mechanisms, clinical indication, and therapeutic values, Cancer Invest 2006, 24:696-703.
Furman, et al., Studies of the Metabolism of Radioiodinated Human Serum Alpha Lipoprotein in Normal and Hyperlipidemic Subjects, J Lab Clin Med 1964, 63:193-204.
Gardian, et al., Analysis of Pancreatic Cancer Microenvironment: Role of Macrophage Infiltrates and Growth Factors Expression, J Cancer 2012, 3:285-291.
Groblewska, et al., Serum levels of granulocyte colony-stimulating factor (G-CSF) and macrophage colony-stimulating factor (M-CSF) in pancreatic cancer patients, Clin Chem Lab Med 2007, 45:30-34.
Gupta, et al., Oral delivery of therapeutic proteins and peptides: a review on recent developments, Drug Deliv 2013, 20:237-246.
Hariharan, et al., Analysis of mortality rates for pancreatic cancer across the world, HPB (Oxford) 2008, 10:58-62.
Ho, et al., TREM-1 expression in tumor-associated macrophages and clinical outcome in lung cancer, Am J Respir Crit Care Med 2008, 177:763-770.
Jinushi and Komohara, Tumor-associated macrophages as an emerging target against tumors: Creating a new path from bench to bedside, Biochim Biophys Acta 2015, 1855:123-130.
Julovi, et al., Apolipoprotein A-II Plus Lipid Emulsion Enhance Cell Growth via SR-B1 and Target Pancreatic Cancer In Vitro and In Vivo, PLoS One 2016, 11:e0151475.
Komohara, et al., Tumor-associated macrophages: Potential therapeutic targets for anti-cancer therapy, Adv Drug Deliv Rev 2016, 99:180-185.
Kubota, et al., M-CSF inhibition selectively targets pathological angiogenesis and lymphangiogenesis, J Exp Med 2009, 206:1089-1102.
Kumari, et al., Role of interleukin-6 in cancer progression and therapeutic resistance, Tumour Biol 2016, 37:11553-11572.
Kuwahara, et al., Expressions of angiogenic factors in pancreatic ductal carcinoma: a correlative study with clinicopathologic parameters and patient survival, Pancreas 2003, 26:344-349.
Lagler, et al., TREM-1 activation alters the dynamics of pulmonary IRAK-M expression in vivo and improves host defense during pneumococcal pneumonia, J Immunol 2009, 183:2027-2036.
Lewis and Pollard, Distinct role of macrophages in different tumor microenvironments, Cancer Res 2006, 66:605-612.
Lin, et al., Macrophages regulate the angiogenic switch in a mouse model of breast cancer, Cancer Res 2006, 66:11238-11246.
Lin, et al., Colony-stimulating factor 1 promotes progression of mammary tumors to malignancy, J Exp Med 2001, 193:727-740.
Liu, et al., The effects of mutations in helices 4 and 6 of ApoA-I on scavenger receptor class B type I (SR-BI)-mediated cholesterol efflux suggest that formation of a productive complex between reconstituted high density lipoprotein and SR-BI is required for efficient lipid transport, J Biol Chem 2002, 277:21576-21584.
Mitchem, et al., Targeting tumor-infiltrating macrophages decreases tumor-initiating cells, relieves immunosuppression, and improves chemotherapeutic responses, Cancer Res 2013, 73:1128-1141.
Pelham and Agrawal, Emerging roles for triggering receptor expressed on myeloid cells receptor family signaling in inflammatory diseases, Expert Rev Clin Immunol 2014, 10:243-256.
Pieramici and Rabena, Anti-VEGF therapy: comparison of current and future agents, Eye (Lond) 2008, 22:1330-1336.
Schenk, et al., TREM-1-expressing intestinal macrophages crucially amplify chronic inflammation in experimental colitis and inflammatory bowel diseases, J Clin Invest 2007, 117:3097-3106.
Schneider, et al., Pancreatic cancer: basic and clinical aspects, Gastroenterology 2005, 128:1606-1625.
Shen and Sigalov, Rationally designed ligand-independent peptide inhibitors of TREM-1 ameliorate collagen-induced arthritis, J Cell Mol Med 2017:in press.
Shen and Sigalov, SARS Coronavirus Fusion Peptide-Derived Sequence Suppresses Collagen-Induced Arthritis in DBA/1J Mice, Sci Rep 2016, 6:28672.
Shen, et al., Diagnostic Magnetic Resonance Imaging of Atherosclerosis in Apolipoprotein E Knockout Mouse Model Using Macrophage-Targeted Gadolinium-Containing Synthetic Lipopeptide Nanoparticles, PLoS One 2015, 10:e0143453.
Shih, et al., Tumor-Associated Macrophage: Its Role in Cancer Invasion and Metastasis, J Cancer Mol 2006, 2:101-106.
Sigalov, Nature-inspired nanoformulations for contrast-enhanced in vivo MR imaging of macrophages, Contrast Media Mol Imaging 2014, 9:372-382.
Sigalov, A novel ligand-independent peptide inhibitor of TREM-1 suppresses tumor growth in human lung cancer xenografts and prolongs survival of mice with lipopolysaccharide-induced septic shock, Int Immunopharmacol 2014, 21:208-219.
Sigalov, Novel mechanistic concept of platelet inhibition, Expert Opin Ther Targets 2008, 12:677-692.
Sigalov, Novel mechanistic insights into viral modulation of immune receptor signaling, PLoS Pathog 2009, 5:e1000404.
Solinas, et al., Tumor-associated macrophages (TAM) as major players of the cancer-related inflammation, J Leukoc Biol 2009, 86:1065-1073.
Tjomsland, et al., Interleukin 1alpha sustains the expression of inflammatory factors in human pancreatic cancer microenvironment by targeting cancer-associated fibroblasts, Neoplasia 2011, 13:664-675.
Varney, et al., Tumour-associated macrophage infiltration, neovascularization and aggressiveness in malignant melanoma: role of monocyte chemotactic protein-1 and vascular endothelial growth factor-A, Melanoma Res 2005, 15:417-425.
Walker and Ko, Beyond first-line chemotherapy for advanced pancreatic cancer: an expanding array of therapeutic options?, World J Gastroenterol 2014, 20:2224-2236.
Wang, et al., T cell antigen receptor (TCR) transmembrane peptides colocalize with TCR, not lipid rafts, in surface membranes, Cell Immunol 2002, 215:12-19.
Wang, et al., Expression of TREM-1 mRNA in acute pancreatitis, World J Gastroenterol 2004, 10:2744-2746.
Wang, et al., Immunohistochemistry in the evaluation of neovascularization in tumor xenografts, Biotech Histochem 2008, 83:179-189.
Weber, et al., TREM-1 Deficiency Can Attenuate Disease Severity without Affecting Pathogen Clearance, PLoS Pathog 2014, 10:e1003900.
Yako, et al., Cytokines as Biomarkers of Pancreatic Ductal Adenocarcinoma: A Systematic Review, PLoS One 2016, 11:e0154016.
Zhu, et al., CSF1/CSF1R Blockade Reprograms Tumor-Infiltrating Macrophages and Improves Response to T-cell Checkpoint Immunotherapy in Pancreatic Cancer Models, Cancer Res 2014, 74:5057-5069.
Zumsteg, et al., Myeloid cells contribute to tumor lymphangiogenesis, PLoS One 2009, 4:e7067.

### Example 6: Mouse Model of LPS-Induced Endotoxemia and In Vivo Survival and Cytokine Release Studies

Animal survival studies and studies of in vivo cytokine release were performed in a mouse model of LPS-induced septic shock using the standard, well known in the art methods as described in Sigalov. Int Immunopharmacol 2014, 21:208-219.

Naïve, female C57BL/6 mice at 8 to 10 weeks of age (18 to 21 g) from the Jackson Laboratory were randomly grouped (10 mice per group) and i.p. injected with vehicle or the indicated doses of dexamethasone (DEX), control peptide GF9-G, GF9, control peptide TRIOPEP-A or TREM-1/TRIOPEP in either free or SLP-bound form. One hour later, mice received i.p. injection of 30 mg/kg LPS from E. coli 055:B5 (Sigma). In some experiments, all formulations were i.p. administered 1 and 3 h after LPS injection. The viability of mice was examined hourly. Body weights were measured daily. In all of the animal experiments, blood samples were collected via a sub-mandibular (cheek) bleed at 90 min after administration of LPS. Statistical analysis of survival curves was performed by the Kaplan-Meier test. Comparisons were made using two-tailed Student's t test. The production of cytokines in serum was measured by a standard sandwich cytokine ELISA procedure using TNF-alpha, IL-1beta and IL-6 ELISA kits (Pierce Biotechnology, Thermo Scientific, Rockford, Ill.) according to the instructions of the manufacturer. Statistical significances in cytokine analysis ELISA data were determined by two-tailed Student's t test.

This example demonstrates that GF9 and TREM-1/TRIOPEP in free or SLP-bound form inhibit LPS-stimulated cytokine production in vivo. This example further demonstrates that GF9 and TREM-1/TRIOPEP in free or SLP-bound form protect mice from LPS-induced septic shock and prolongs survival of septic mice. This example further demonstrates that the magnitude of this effect can depend on dose and administration time schedule and whether GF9 and TREM-1/TRIOPEP are administered in free or SLP-bound form. See FIGS. 18A-D.

### Example 7: Lung Cancer Tumor Xenografts in Nude Mice and In Vivo Tumor Growth Studies

Animal efficacy studies were performed in human xenograft mouse models of NSCLC using female 6-8 week old NU/J mice from the Jackson Laboratory (Bar Harbor, Me.) using the standard, well-known in the art methods as described in Sigalov. Int Immunopharmacol 2014, 21:208-219 and disclosed in Wu, et al. U.S. Pat. No. 8,415,453 and Sigalov U.S. Pat. No. 8,513,185, each of which is herein incorporated by reference in it's entirety.

Animal efficacy studies were performed using female 6-8 week old NU/J mice from the Jackson Laboratory (Bar Harbor, Me.). Animals were handled as specified in the USDA Animal Welfare Act (9 CFR, Parts 1, 2, and 3) and as described in the Guide for the Care and Use of Laboratory Animals from the National Research Council. Human lung carcinoma cell lines H292 and A549 were obtained from ATCC. Tumor cells in culture were harvested and resuspended in a 1:1 ratio of RPMI 1640 and Matrigel (BD Biosciences, San Jose, Calif.). NSCLC xenografts were established by injecting subcutaneously into the right flanks 5×10⁶ viable cells per mouse. Tumor volumes were calculated with caliper measurements using the formula V=π/6 (length×width×width). When tumor volumes reached an average of 200 mm³, tumor-bearing animals were randomized into groups of 10, and dosing of GF9 or TREM-1/TRIOPEP in free or SLP-bound form was initiated. All tested formulations were intraperitoneally (i.p.) injected at indicated doses and administration schedule. Clinical observations, body weights and tumor volume measurements were made 3 times weekly. Tumor volumes were analyzed using repeated measures ANOVA followed by Bonferroni test. Data points were represented as mean tumor volume±SEM. Antitumor effects were expressed as the percentage of T/C (treated versus control), dividing the tumor volumes from treatment groups with the control groups and multiplied by 100. According to the National Cancer Institute (NCI) standards (see e.g., Johnson, et al. Br J Cancer 2001, 84:1424-1431), a % T/C≤42 is indicative of antitumor activity. At the end of the experiment, the animals were sacrificed and the tumors were excised and weighed.

This example demonstrates that GF9 or TREM-1/TRIOPEP in free or SLP-bound form inhibits tumor growth in two human NSCLC xenograft mouse models. This example further demonstrates that the magnitude of an anticancer effect can depend on dose and time schedule for administration and whether TREM-1 inhibitory peptides are administered in free or SLP-bound form. This example further demonstrates that GF9 and TREM-1/TRIOPEP in free or SLP-bound form are non-toxic and well-tolerable by cancer mice. See FIGS. 13-16.

### Example 8: Pancreatic Cancer Tumor Xenografts in Nude Mice and In Vivo Tumor Growth Studies

In order to demonstrate that modulation of the TREM-1/DAP-12 signaling pathway using GF9 and TREM-1 TRIOPEP in free form and bound to SLP is effective in inhibiting TREM-1-mediated cell activation and reducing pancreatic tumor (PC) growth, animal efficacy studies were performed in human xenograft mouse models of PC using 5-6 week old female athymic nude-Foxn1^{nu} mice obtained from Envigo (formerly Harlan, Inc.) using the standard, well known in the art methods as described in Shen and Sigalov. Mol Pharm 2017, 14:4572-4582, herein incorporated by referene in it's entirety.

### Animal Studies

Mice were implanted subcutaneously into the right flank with 5×10⁶ AsPC-1, BxPC-3 or Capan-1 cells in equal parts of serum-free growth medium and Matrigel. Mice were monitored daily and tumor measurements were taken along the length and width using Vernier calipers twice weekly until sacrifice. Tumor volumes were calculated using a modified ellipsoidal formula: (Length×Width²)/2. When tumors reached a calculated volume of approximately 150-200 mm³, mice were sorted into treatment groups and i.p. injected intraperitoneally once daily for 5 days per week (5qw) at indicated doses. Treatment persisted for 31 days, 29 days and 29 days for mice containing established AsPC-1, BxPC-3 and Capan-1 xenograft tumors, respectively. Mice were humanely sacrificed when individual tumors exceeded 1500 mm³.

### Immunohistochemistry

All staining and quantification procedures were performed by HistoTox Laboratories. Briefly, mice containing AsPC-1, BxPC-3, and Capan-1 tumors were humanely euthanized for necropsy at the end of the study. Excised tumors were fixed using 10% neutral buffered formalin for 1-2 days, processed for paraffin embedding, and sectioned at 4 m. Antigen retrieval for F4/80 was achieved using Proteinase K (Dako North America). Sections were blocked for peroxidase and alkaline phosphatase activity using Dual Endogenous Enzyme Block (Dako North America). Sections were then incubated with Protein Block (Dako North America) followed by primary antibody F4/80 (1:2000, AbD Serotec) diluted using 1% bovine serum albumin in Tris-buffered saline. Afterward, sections were incubated using EnVision+ secondary antibodies (Dako North America), followed by 3,3′-diaminobenzidine in chromogen solution (Dako North America) and counterstained using hematoxylin (Dako North America). Quantitative analysis of intratumoral F4/80 staining was determined using Visiopharm software.

### Cytokine Detection

Blood was collected on study days 1 and 8 and processed into serum. Serum cytokines were analyzed by Quantibody Mouse Cytokine Array Q1 kits (RayBiotech) according to the manufacturer's instructions. Statistical Analysis. All statistical analyses were performed using GraphPad Prism 6.0 (GraphPad Software). Percent treatment/control (T/C) values were calculated using the following formula: % T/C=100×ΔT/ΔC where T and C are the mean tumor volumes of the drug-treated and control groups, respectively, on the final day of the treatment; ΔT is the mean tumor volume of the drug-treated group on the final day of the treatment minus mean tumor volume of the drug-treated group on initial day of dosing; and ΔC is the mean tumor volume of the control group on the final day of the treatment minus mean tumor volume of the control group on initial day of dosing.

### Statistical Analysis

Results are expressed as the mean±SEM. Statistical differences were analyzed using analysis of variance with Bonferroni adjustment unless otherwise noted. The Kaplan-Meier method was used to estimate survival as a function of time, and survival differences were analyzed by the log-rank test. p values less than 0.05 were considered significant.

This example demonstrates that TREM-1 inhibitory peptide GF9 and TREM-1/TRIOPEP in free or SLP-bound form inhibit tumor growth in three human PC xenograft mouse models. This example further demonstrates that TREM-1 blockade using these formulations improves survival. This example further demonstrates that TREM-1 blockade using these formulations reduces the intratumoral macrophage infiltration and that the magnitude of an anticancer effect can depend on the xenograft and dose and whether GF9 and TREM-1/TRIOPEP are administered in free or SLP-bound form. This example further demonstrates that the anticancer activity of the TREM-1 inhibitory formulations correlates with basal intratumoral macrophage content. The example further demonstrates that TREM-1 blockade using TREM-1 inhibitory peptide GF9 or trifunctional peptides TREM-1/TRIOPEP GA31 and GE31 is accompanied by reduction of serum levels of IL-1α, IL-6 and CSF-1. See FIG. 14B (shown for BxPC-3).

### Example 9: Mouse Tolerability Studies

Mouse tolerability studies were performed in healthy C57BL/6 mice using the standard, well-known in the art methods as described in Sigalov. Int Immunopharmacol 2014, 21:208-219, herein incorporated by reference.

Naïve, female C57BL/6 mice at 8 to 10 weeks of age (18 to 21 g) from the Jackson Laboratory were used. Animals were randomly grouped (5 mice per group) and i.p. injected with increasing doses of GF9 or TREM-1/TRIOPEP in free form. Clinical observations and body weights were made twice daily.

This example demonstrates that TREM-1/TRIOPEP in free form is non-toxic and well tolerated in healthy mice at doses of up to at least 400 mg/kg. This example further demonstrates that GF9 in free form is non-toxic and well tolerated in healthy mice at doses of up to at least 300 mg/kg. See FIG. 16.

### Example 10: Haemodynamic Studies in Septic Rats

The role of GF9 and TREM-1-related trifunctional peptides in further models of septic shock, is investigated by performing LPS- and cecal ligation and puncture (CLP)-induced endotoxinemia experiments in rats. The experiments can be conducted analogously to those described in Gibot, et al. Infect Immun 2006, 74:2823-2830 and disclosed in Faure, et al. U.S. Pat. No. 8,013,116; Faure, et al. U.S. Pat. No. 9,273,111; and Sigalov U.S. Pat. No. 8,513,185, each of which is herein incorporated by reference in it's entirety.

### LPS-Induced Endotoxinemia

Animals are randomly grouped (n=10-20) and treated with Escherichia coli LPS (0111:B4, Sigma-Aldrich, Lyon, France) i.p. in combination with control peptide TREM-1/TRIOPEP-A or TREM-1/TRIOPEP in either free or SLP-bound form at various concentrations.

### CLP Polymicrobial Sepsis Model

Rats (n=6-10 per group) are anesthetized by i.p. administration of ketamine (150 mg/kg). The caecum is exposed through a 3.0-cm abdominal midline incision and subjected to a ligation of the distal half followed by two punctures with a G21 needle. A small amount of stool is expelled from the punctures to ensure potency. The caecum is replaced into the peritoneal cavity and the abdominal incision closed in two layers. After surgery, all rats are injected s.c. with 50 mL/kg of normal saline solution for fluid resuscitation. Control peptide GF9-G, GF9, control peptide TRIOPEP-A or TREM-1/TRIOPEP in either free or SLP-bound form are then administered at various concentrations.

### Haemodynamic Measurements in Rats

Immediately after LPS administration as well as 16 hours after CLP, arterial BP (systolic, diastolic, and mean), heart rate, abdominal aortic blood flow, and mesenteric blood flow are recorded. Briefly, the left carotid artery and the left jugular vein are cannulated with PE-50 tubing. Arterial BP is continuously monitored by a pressure transducer and an amplifier-recorder system (IOX EMKA Technologies, Paris, France). Perivascular probes (Transonic Systems, Ithaca, N.Y.) are wrapped up the upper abdominal aorta and mesenteric artery, allowed to monitor their respective flows by means of a flowmeter (Transonic Systems). After the last measurement (4^{th} hour after LPS and 24^{th} hour after CLP), animals are sacrificed by an overdose of sodium thiopental i.v.

### Biological Measurements

Blood is sequentially withdrawn from the left carotid artery. Arterial lactate concentrations and blood gases analyses are performed on an automatic blood gas analyser (ABL 735, Radiometer, Copenhagen, Denmark). Concentrations of TNF-alpha and IL-1beta in the plasma are determined by an ELISA test (Biosource, Nivelles, Belgium) according to the recommendations of the manufacturer. Plasmatic concentrations of nitrates/nitrites are measured using the Griess reaction (R&D Systems, Abingdon, UK).

### Statistical Analyses

Between-group comparisons are performed using Student's t tests. All statistical analyses are completed with Statview software (Abacus Concepts, Calif.).

### Example 11: Attenuation of Intestinal Inflammation in Animal Models of Colitis

In order to demonstrate that the GF9 and TREM-1-related TRIOPEP formulations are effective in inhibiting TREM-1-mediated cell activation in animal models of colitis, the experiments can be conducted analogously to those described in Schenk, et al. J Clin Invest 2007, 117:3097-3106 and disclosed in Faure, et al. U.S. Pat. No. 8,013,116; Faure, et al. U.S. Pat. No. 9,273,111 and Sigalov U.S. Pat. No. 8,513,185, each of which is herein incorporated by reference in it's entirety.

### Mice

C57BL/6 mice, purchased from Harlan, and C57BL/6 RAG2−/− mice, bred in a specific pathogen-free (SPF) animal facility, are used at 8-12 weeks of age. All experimental mice are kept in micro-isolator cages in laminar flows under SPF conditions.

### Mouse Models of Colitis

For experiments involving the adoptive T cell transfer model, colitis is induced in C57BL/6 RAG2−/− mice by adoptive transfer of sorted CD4+CD45RBhigh T cells. Briefly, CD4+ T cells are isolated from splenocytes from C57BL/6 mice, and after osmotic lysis of erythrocytes, CD4+ T cells are enriched by a negative MACS procedure for CD8alpha and B220 (purified, biotinylated, hybridoma supernatant) using avidin-labeled magnetic beads (Miltenyi Biotec). Subsequently, the CD4+ T cell-enriched fraction is stained and FACS sorted for CD4+(RM4-5; BD Biosciences—Pharmingen), CD45RBhi (16A; BD Biosciences—Pharmingen), and CD25− (PC61; eBioscience) naive T cells. Each C57BL/6 RAG2−/− mouse is injected i.p. with 1×105 syngeneic CD4+CD45RBhighCD25− T cells. Colitic mice are sacrificed and analyzed on day 14 after adoptive transfer.

For experiments involving the dextran sodium sulfate (DSS) colitis model, C57BL/6 mice are given autoclaved tap water containing 3% DSS (DSS salt, reagent grade, mol wt: 36-50 kDa; MP Biomedicals) ad libitum over a 5-day period. The consumption of 3% DSS is measured. DSS is replaced thereafter by normal drinking water for another 4 days. Mice are euthanized and analyzed at the end of the 9-day experimental period.

Treatment with GF9, TREM-1/TRIOPEP and TREM-1/TRIOPEP-SLP

Upon colitis induction, either starting on day 0 or after onset of colitis on day 3 (as indicated), mice are treated with either a control peptide GF9-G, GF9, a control peptide TREM-1/TRIOPEP-A or TREM-1/TRIOPEP in free or SLP-bound form i.p. injected at various concentrations in 200 ul saline.

### Colitis Scoring

At the end of the experiments, the colon length is measured from the end of the cecum to the anus. Fecal samples are tested for occult blood using hemo FEC (Roche) tests (score 0, negative test; 1, positive test and no rectal bleeding; 2, positive test together with visible rectal bleeding). The colon is divided into 2 parts. From each mouse, identical segments from the distal and proximal colon are taken for protein and RNA isolation and histology, and frozen tissue blocks are prepared for subsequent analysis. Histological scoring of paraffin-embedded H&E-stained colonic sections is performed in a blinded fashion independently by 2 pathologists. To assess the histopathological alterations in the distal colon, a scoring system is established using the following parameters: (a) mucin depletion/loss of goblet cells (score from 0 to 3); (b) crypt abscesses (score from 0 to 3); (c) epithelial erosion (score from 0 to 1); (d) hyperemia (score from 0 to 2); (e) cellular infiltration (score from 0 to 3); and (f) thickness of colonic mucosa (score from 1 to 3). These individual histology scores are added to obtain the final histopathology score for each colon (0, no alterations; 15, most severe signs of colitis).

### RNA Isolation and RT-PCR

RNA is isolated from intestinal tissue samples preserved in RNAlater (QIAGEN), using the RNAeasy Mini Kit (QIAGEN). RT-PCR is performed with 400 ng RNA each, using the TaqMan Gold RT-PCR Kit (Applied Biosystems). Primers are designed as follows: mouse TREM-1, forward 5′-GAGCTTGAAGGATGAGGAAGGC-3′ and reverse 5′-CAGAGTCTGTCACTTGAAGGTCAGTC-3′; mouse TNF, forward 5′-GTAGCCCACGTCGTAGCAAA-3′ and reverse 5′-ACGGCAGAGAGGAGGTTGAC-3′; mouse beta-actin, forward 5′-TGGAATCCTGTGGCATCCATGAAAC-3′ and reverse 5′-TAAAACGCAGCTCAGTAACAGTCCG-3′; human TREM-1, forward 5′-CTTGGTGGTGACCAAGGGTTTTTC-3′ and reverse 5′-ACACCGGAACCCTGATGATATCTGTC-3′; human TNF, forward 5′-GCCCATGTTGTAGCAAACCC-3′ and reverse 5′-TAGTCGGGCCGATTGATCTC-3′; human GAPDH, forward 5′-TTCACCACCATGGAGAAGGC-3′ and reverse 5′-GGCATGGACTGTGGTCATGA-3′. PCR products are semiquantitatively analyzed on agarose gels.

Human TREM-1 and mouse TREM-1 and TNF expression is also assessed by real-time PCR using the TREM-1 QuantiTect primer assay system and QuantiTect SYBR green PCR Kit (both from QIAGEN). GAPDH is used to normalize TREM-1 and TNF expression levels. DNA is amplified on a 7500 Real-Time PCR system (Applied Biosystems), and the increase in gene expression is calculated using Sequence Detection System software (Applied Biosystems).

### Western Blot Analysis

Protein samples are separated on a denaturing 12% acrylamide gel, followed by transfer to nitrocellulose filter and probing with the primary antibody. Anti-TREM-1 (polyclonal goat IgG, 0.1 ug/ml; R&D Systems) or anti-tubulin (clone B-5-1-2, 1:5,000; Sigma-Aldrich) is used as primary reagent. As secondary antibodies, HRP-labeled donkey anti-goat Ig (1:2,000; The Binding Site) and goat anti-mouse Ig (1:4,000; Sigma-Aldrich) are used. Binding is detected by chemiluminescence using a Super Signal West Pico Kit (Pierce).

### Statistics

The unpaired 2-tailed Student t test is used to compare groups; P values less than 0.05 are considered significant.

### Example 12: Autophage Activity and Colitis in Mice

In order to further demonstrate that the GF9 and TREM-1-related TRIOPEP formulations are effective in inhibiting TREM-1-mediated cell activation in animal models of colitis, the experiments can be conducted analogously to those described in Kokten, et al. J Crohns Colitis 2018, 12:230-244 and disclosed in Faure, et al. U.S. Pat. No. 8,013,116; and Faure, et al. U.S. Pat. No. 9,273,111, each of which is herein incorporated by reference in it's entirety.

### Animals

In vivo experiments are performed as recommended by the US National Committee on Ethics Reflection Experiment [described in the Guide for Care and Use of Laboratory Animals, NIH, MD, 1985]. The experiments are performed on 25 adult male C57BL/6 mice [Janvier Labs, Le Genest-Saint-Isle, France] and 10 adult male Trem-1 knock-out [TREM-1 KO] C57BL/6 mice [INSERM U1116, Inotrem Laboratory, Nancy, France], all aged between 7 and 9 weeks. The animals are housed at 22-23° C., with a 12 h/12 h light/dark cycle, and ad libitum access to food and water.

Induction of colitis, treatment with GF9 and TREM-1/TRIOPEP and assessment of disease activity index. Colitis is induced by administration of 3% dextran sulfate sodium [DSS, molecular weight 36,000-50,000, MP Biomedical, Strasbourg, France] dissolved in water for 5 days. DSS is replaced thereafter by normal drinking water for another 5 days. Either a control peptide GF9-G, GF9, a control peptide TREM-1/TRIOPEP-A or TREM-1/TRIOPEP in free or SLP-bound form or the vehicle alone, used as control, are i.p. administered 2 days before colitis induction and then once daily until the last day of DSS administration, at different concentrations in 200 μL of saline. This dose is chosen after having performed dose-response experiments. Bodyweight, physical condition, stool consistency, water/food consumption and the presence of gross and occult blood in excreta and at the anus are determined daily. The DAI is also calculated daily by scoring bodyweight loss, stool consistency and blood in the stool on a 0 to 4 scale. 41 The overall index corresponds to the weight loss, stool consistency and rectal bleeding scores divided by three, and thus ranges from 0 to 4.

### Collection of Colon Tissue and Fecal Samples

Ten days after the initiation of colitis with DSS, the mice are sacrificed by decapitation. The colon is quickly removed, opened along its length and gently washed in PBS [2.7 mmol/L KCl, 140 mmol/L NaCl, 6.8 mmol/L Na2HPO4.2H2O, 1.5 mmol/L KH2PO4, pH 7.4]. For histological assessment samples are fixed overnight at 4° C. in 4% paraformaldehyde solution and embedded in paraffin. For protein extractions samples are frozen in liquid nitrogen [−196° C.] and stored at −80° C. For the gut microbiota analysis, whole fecal pellets are collected daily in sterile tubes and immediately frozen at −80° C. until analysis.

### Histological Assessment and Scoring

Colitis is histologically assessed on 5 m sections stained with hematoxylin-eosin-saffron [HES] stain. The histological colitis score is calculated blindly by an expert pathologist.

### Endoscopic Assessment and Scoring

Endoscopy is performed on the last day of the study, just before the mice are sacrificed. Prior to the endoscopic procedure, mice are anaesthetized by isoflurane inhalation. The distal colon [3 cm] and the rectum are examined using a rigid Storz Hopkins II miniendoscope [length: 30 cm; diameter: 2 mm; Storz, Tuttlingen, Germany] coupled to a basic Coloview system [with a xenon 175 light source and an Endovision SLB Telecam; Storz]. Air is insufflated via a 9-French gauge over-tube and a custom, low-pressure pump with manual flow regulation [Rena Air 200; Rena, Meythet, France]. All images are displayed on a computer monitor and recorded with video capture software [Studio Movie Board Plus from Pinnacle, Menlo Park, Calif.]. The endoscopy score is calculated from three subscores: the vascular pattern [scored from 1 to 3], bleeding [scored from 1 to 4] and erosions/ulcers [scored from 1 to 4].

### Western Blot Analysis

Total protein is extracted from the frozen colon samples by lysing homogenized tissue in a radioimmunoprecipitation assay [RIPA] buffer [0.5% sodium deoxycholate, 0.1% sodium dodecyl sulfate [SDS] and 1% NP-40] supplemented with protease inhibitors [Roche Diagnostics, Mannheim, Germany]. Protein is then quantified using the bicinchoninic acid assay method. For each mouse, a total of 20 μg of protein is transferred to a 0.45 m polyvinylidene fluoride [PVDF] or 0.45 m nitrocellulose membrane following electrophoretic separation on a denaturing acrylamide gel. The membrane is blocked with 5% w/v non-fat powdered milk or 5% w/v bovine serum albumin [BSA] diluted in Tris-buffered saline with 0.1% v/v Tween® 20 [TBST] for 1 h at room temperature. The PVDF or nitrocellulose membranes are then incubated overnight at 4° C. with various primary antibodies diluted in either 5% w/v nonfat powdered milk or 5% w/v BSA, TBST. After washing in TBST, the appropriate HRP-conjugated secondary antibody is added and the membrane is incubated for 1 h at room temperature. After further washing in TBST, the proteins are detected using an ECL or ECL PLUS kit [Amersham, Velizy-Villacoublay, France]. Glyceraldehyde 3-phosphate dehydrogenase [GAPDH] is used as an internal reference control.

Enzyme-Linked Immunosorbent Assay [ELISA] for Analysis of Soluble TREM-1 [sTREM-1]

At the time of animal sacrifice, whole blood from each mouse is collected into heparinized tubes. These tubes are centrifuged at 3,000 g for 10 min at 4° C. to collect the supernatants, which are stored at −80° C. until use. Plasma concentration of sTREM-1 is determined by a sandwich ELISA technique using the Quantikine kit assay [RnD Systems, Minneapolis, Minn., USA] according to the manufacturers' instructions. Briefly, samples are incubated with a monoclonal antibody specific for TREM-1 pre-coated onto the wells of a microplate. Following a wash, to eliminate the unbound substances, an enzyme-linked polyclonal antibody specific for TREM-1 is added to the wells. After washing away the unbound conjugate, a substrate solution is added to the wells. Color development is stopped and optical density of each well is determined within 30 min using a microplate reader [Sunrise, Tecan, Mannedorf, Switzerland] set to 450 nm, with a wavelength correction set to 540 nm. All measurements are performed in duplicate and the sTREM-1 concentration is expressed in pg/ml.

Reverse transcription-quantitative polymerase chain reaction Total RNA is purified from the frozen colon samples with the RNeasy Lipid Tissue kit following the recommendation of Qiagen [Courtaboeuf, France], which includes treatment with DNase. To check for possible DNA contamination of the RNA samples, reactions are also performed in the absence of Omniscript RT enzyme [Qiagen]. Reverse transcription is performed using PrimeScript™ RT Master Mix [TAKARA Bio, USA] according to the manufacturer's recommendations with 200 ng of RNA in a 10 μL reaction volume. PCR is then carried out from 2 μL of cDNA with SYBR® Premix Ex Taq™ [Tli RNaseH Plus] [TAKARA Bio, USA] according to the manufacturer's recommendations in a 20 μL reaction volume, with reverse and forward primers at a concentration of 0.2 μM. Specific amplifications are performed using the following primers: TREM-1, forward 5′-CTGTGCGTGTTCTTTGTC-3′ and reverse 5′-CTTCCCGTCTGGTAGTCT-3′. Quantification is performed with RNA polymerase II [Pol II] as an internal standard with the following primers: forward 5′-AGCAAGCGGTTCCAGAGAAG-3′ and reverse 5′-TCCCGAACACTGACATATCTCA-3′. Temperature cycling for TREM-1 is 30 s at 95° C. followed by 40 cycles consisting of 95° C. for 5 s and 59° C. for 30 s. Temperature cycling for RNA polymerase II is 30 s at 95° C. followed by 40 cycles consisting of 95° C. for 5 s and 60° C. for 30 s. Results are expressed as arbitrary units by calculating the ratio of crossing points of amplification curves of TREM-1 and internal standard by using the δδCt method.

### Microbiota Analysis

For the pharmacologically [with TREM-1/TRIOPEP treatment] inhibition of TREM-1, total DNA is extracted from three pooled fecal pellets from each group of mice [day 0 to day 10; n=33 samples]. For microbiota analysis by MiSeq sequencing, the V3-V4 region [519F-785R] of the 16S rRNA gene is amplified with the primer pair S-DBact-0341-b-S-17/S-D-Bact-0785-a-A-21.45 The following quality filters are applied: minimum length=300 base pairs [bp], maximum length=600 bp and minimum quality threshold=20. This filtering yields an average [range] of 25600 reads/samples [14,553-35,490] for further analysis. High-quality reads are pooled, checked for chimeras [using uchime46], and grouped into operational taxonomic units [OTUs][based on a 97% similarity threshold] using USEARCH 8.0.47 Singletons and OTUs representing less than 0.02% of the total number of reads are removed, and the phylogenetic affiliation of each OTU is assessed with Ribosomal Database Project's taxonomy48 from the phylum level to the species level. The mean [range] number of detected OTUs per sample is 324 [170-404]. In the experiments involving Trem-1 KO mice, similar methods are applied but total DNA is extracted from individual fecal pellets of each mouse from the four groups of animals at baseline [before DSS treatment] and at day 10 [after DSS treatment] [n=37 samples]. Following MiSeq sequencing of the V3-V4 region of the 16S rRNA gene, yielding 2,143,457 raw reads, quality filtering is applied [minimum length=200 bp, maximum length=600 bp and minimum quality threshold=20] and an average [range] of 11,560 reads/samples [7,560-18,495] is kept for further analysis. The mean [range] number of detected OTUs per sample is 599 [131-798].

### Statistical Analysis

A two-tailed Student t test is used to compare two groups and a one-way analysis of variance [ANOVA] is used to compare three or more groups. Bonferroni or Tamhane post hoc tests are applied, depending on the homogeneity of the variance. The threshold for statistical significance is set to p<0.05. The statistical language R is used for data visualization and to perform abundance-based principal component analysis [PCA] and interclass PCA associated with Monte-Carlo rank testing on the bacterial genera.

### Example 13: Modulation of the TREM-1 Pathway During Severe Hemorrhagic Shock in Rats

In order to demonstrate that the GF9 and TREM-1-related TRIOPEP formulations are effective in inhibiting TREM-1-mediated cell activation and preventing organ dysfunction and improving survival in rats during severe hemorrhagic shock, the experiments can be conducted analogously to those described in Gibot, et al. Shock 2009, 32:633-637 and disclosed in Faure, et al. U.S. Pat. No. 8,013,116; Faure, et al. U.S. Pat. No. 9,273,111, and Sigalov. U.S. Pat. No. 8,513,185.

### Animals

Adult male Wistar rats (250-300 g) are purchased from Charles River Laboratories (Wilmington, Mass., USA). After 1 week of acclimatization, rats are fasted 12 h before the experiments and are allowed free access to water. All the studies described in the succeeding sentences comply with the regulations concerning animal use and care published by the National Institutes of Health.

### GF9 and TREM-1-Related TRIOPEP Formulations

Control peptide GF9-G, GF9, control peptide TREM-1/TRIOPEP-A and TREM-1-related TRIOPEP in free and SLP-bound form are synthesized as described herein.

### Hemorrhagic Shock Model

Hemorrhagic shock is induced by bleeding from a heparinized (10 UI/mL) carotid artery catheter. Briefly, the rats are anesthetized (50 mg/kg pentobarbital sodium, i.p.) and kept on a temperature-controlled surgical board (37° C.). A tracheostomy is performed, and the animals are ventilated supine (tidal volume, 7-8 mL/kg; rodent ventilator no. 683; Harvard Apparatus, Holliston, Mass.) with a fraction of inspired oxygen of 0.3 and a respiratory rate of 60 breaths per minute. Anesthesia and respiratory support are maintained during the whole experiment. The left carotid artery and the left jugular vein are cannulated with PE-50 tubing. Arterial blood pressure is continuously monitored by a pressure transducer and an amplifier-recorder system (IOX EMKA Technologies, Paris, France). After a 30-min stabilization period, blood is drawn in 10 to 15 min via the carotid artery catheter until MAP reached 40 mmHg. Blood is kept at 37° C., and MAP is maintained between 35 and 40 mm Hg during 60 min. Rats are then allocated randomly (n=10-12 per group) to receive 0.1 mL of either saline (isotonic sodium chloride solution), GF9-G, GF9, TREM-1/TRIOPEP-A or TREM-1/TRIOPEP in free or SLP-bound form at various concentrations in 0.1 mL of saline solution over 1 min via the jugular vein (H0). Shed blood and ringer lactate (volume=3× shed volume) are then infused via the jugular vein in 60 min, and rats are observed for a 4-h period before being killed by pentobarbital sodium overdose. Killing occurs earlier if MAP decreased to less than 35 mm Hg.

### Arterial Blood Gas, Lactate, and Cytokines

Arterial blood gas and lactate concentrations are determined hourly on an automatic blood gas analyzer (ABL 735; Radiometer, Copenhagen, Denmark). Concentrations of TNF-alpha and IL-6 and sTREM-1 in the plasma are determined in triplicate by enzyme-linked immunosorbent assay (Biosources, Nivelles, Belgium; RnD Systems, Lille, France).

### Bacterial Translocation

Rats are killed under anesthesia, and mesenteric lymph node (MLN) complex, spleen, and blood are aseptically removed 4 h after the beginning of reperfusion (or earlier if MAP decreased <35 mm Hg). Homogenates of MLN and spleen and serial blood dilutions are plated and incubated overnight at 37° C. on Columbia blood agar plates (in carbon dioxide and anaerobically) and Macconkey agar (in air). Visible colonies are then counted.

### Pulmonary Integrity

Additional groups of rats (n=4) are subjected to the same procedure but are also infused via the tail vein with fluorescein isothiocyanate (FITC)-albumin (5 mg/kg in 0.3 mL of phosphate-buffered saline) 2 h after the beginning of reperfusion. Rats in these groups are killed 2 h later with an overdose of sodium pentobarbital (200 mg/kg). Immediately thereafter, the lungs are lavaged three times with 1 mL of phosphate-buffered saline, and blood is collected by cardiac puncture. The bronchoalveolar lavage fluid (BALF) is pooled, and plasma is collected. Fluorescein isothiocyanate-albumin concentrations in BALF and plasma are determined fluorometrically (excitation, 494 nm; emission, 520 nm). The BALF-plasma fluorescence ratio is calculated and used as a measure of damage to pulmonary alveolar endothelial/epithelial integrity as previously described (Yang et al. Crit Care Med 2004; 32:1453-9).

### Statistical Analysis

Data are analyzed using ANOVA or ANOVA for repeated measures when appropriate, followed by Newman-Keuls post hoc test. Survival curves are compared using the log-rank test. A two-tailed value of P less than 0.05 is deemed significant. All analyses are performed with GraphPad Prism software (GraphPad, San Diego, Calif.).

### Example 14: Pharmacological Inhibition of TREM-1 in Experimental Atherosclerosis

In order to further demonstrate that GF9 and the TREM-1-related TRIOPEP formulations are effective in inhibiting TREM-1-mediated cell activation in animal models of atherosclerosis, the experiments can be conducted analogously to those described in Joffre, et al. J Am Coll Cardiol 2016, 68:2776-2793 and disclosed in Faure, et al. U.S. Pat. No. 8,013,116 and Faure, et al. U.S. Pat. No. 9,273,111.

### Animals

Trem-1^{-/-} mice (null for the Trem-1 gene) are generated (GenOway, Lyon, France) and backcrossed for more than 10 generations into a C57BL/6J background. Ten-week-old male C57BL/6J Ldlr^{-/-} mice are subjected to medullar aplasia by lethal total body irradiation (9.5 Gy). The mice are repopulated with an intravenous injection of bone marrow cells isolated from femurs and tibias of sex-matched C57BL/6J Trem-1^{-/-} mice or Trem-1^{+/+} littermates. After 4 weeks of recovery, mice are fed a proatherogenic diet containing 15% fat, 1.25% cholesterol, and 0% cholate for 4, 8, or 14 weeks. Eight-week old male ApoE^{-/-} mice are blindly randomized and treated daily by i.p. injection of GF9-G, GF9, TREM-1/TRIOPEP-A or TREM-1/TRIOPEP in free or SLP-bound form at various concentrations during 4 weeks and were put on either a chow or a high-fat diet (15% fat, 1.25% cholesterol).

### Extent and Compositions of Atherosclerotic Lesions

Plasma cholesterol is measured using a commercial cholesterol kit. The basal half of the ventricles and ascending aorta are perfusion-fixed in situ with 4% paraformaldehyde. Afterward, they are removed, transferred to a phosphate-buffered saline (PBS)-30% sucrose solution, embedded in frozen optimal cutting temperature compound and stored at −70° C. Serial 10-μm sections of the aortic sinus with valves (80 per mouse) are cut on a cryostat. One of every 5 sections is kept for plaque size quantification after Oil Red O (Sigma-Aldrich, St. Louis, Mo.) staining. Thus, 16 sections, spanning an 800-μm length of the aortic root, are used to determine mean lesion area for each mouse. Oil Red O-positive lipid contents are quantified by a blinded operator using HistoLab software (Microvisions Instruments, Paris France), which is also used for morphometric studies. En face quantification is used for atherosclerotic plaques along the thoracoabdominal aorta. The aorta is flushed with PBS through the left ventricle and removed from the root to the iliac bifurcation. Then, the aorta is fixed with 10% neutral-buffered formalin. After a thorough washing, adventitial tissue is removed, and the aorta opened longitudinally to expose the luminal surface. Afterward, the aorta, as one tissue example, is stained with Oil Red O for visualizing with the atherosclerotic lesions, as one disease example, quantified by a blinded operator. Collagen is detected using Sirius red stain, and necrotic core is quantified after Masson's trichrome staining. Macrophage presence is determined using specific antibodies. At least 4 sections per mouse are examined for each immunostaining, and appropriate negative controls are used. For immunostaining of mouse atherosclerotic plaques, as one example of mouse tissue, antibodies against Trem-1 (Bs 4886R), macrophage/monocyte antibody (MOMA)-2 (specifically MAB1852), Ly6G, (1A8), and CD3 (A0452) are used. Terminal dUTP nick end-labeling (TUNEL) staining is performed using histochemistry and fluorescent staining. Total proteins are extracted from human atherosclerotic plaque, as one tissue example, and TREM-1 protein level is quantified by Luminex (Thermo Fischer Scientific).

Cells are cultured in RPMI 1640 medium supplemented with L-alanyl-L-glutamine dipeptide (Glutamax, Thermo Fisher Scientific), 10% fetal calf serum, 0.02 mM b-mercaptoethanol, and antibiotics. For cytokine measurements, splenocytes are stimulated with lipopolysaccharide (LPS) (10 μg/ml) and interferon (IFN)-gamma (100 UI/ml) for 24 or 48 h. IL-10, IL-12, and TNF-α production in the supernatants is measured using specific enzyme-linked immunosorbent assays (ELISA).

Primary macrophages are derived from mouse bone marrow-derived cells (BMDM). Tibias and femurs of C57B16/J male mice are dissected, and their marrow is flushed out. Cells are grown for 7 days at 37° C. in a solution of RPMI 1640 medium, 20% neonatal calf serum, and 20% macrophage-colony-stimulating factor-rich L929-conditioned medium. To analyze oxidized LDL (oxLDL) uptake, BMDMs are exposed to human oxLDL (25 μg/ml) for 24 and 48 h. Cells are washed, fixed, and stained using Red Oil. Foam cells are quantified blindly on 6 to 8 fields, and the mean is recorded. To analyze macrophage phenotype, BMDMs are stimulated with LPS (10 μg/ml) and IFN-g (100 UI/ml) for 24 h. IL-10, IL-12, IL-1b, and TNF-α production in the supernatant is measured using ELISA. To analyze apoptosis susceptibility, macrophages are incubated with TNF-α (10 ng/ml) and cycloheximide (10 μmol/l) for 6 h or etoposide (50 μmol/l) for 12 h, or in a fetal calf serum-free medium. Apoptosis is determined by independent experiments using Annexin V fluorescein isothiocyanate apoptosis detection kit with 7-AAD (APC, BD Biosciences, San Jose, Calif.) according to the manufacturer's instructions.

Human monocytes are isolated using anti-CD14 microbeads from healthy donors. Cells are cultured with macrophage colony-stimulating factor (50 ng/ml) for 7 days to induce mature macrophages. Nonclassical monocytes are labeled in vivo by retro-orbital intravenous injection of 1 mm fluorescent microsphere diluted to one-quarter in sterile PBS. Chimeric Ldlr^{-/-} [symbol] mice were euthanized 48 h later, and cell labeling is checked by flow cytometry. Beads that reflect monocyte recruitment are quantified in 8 aortic sinus sections per mouse.

### Statistical Analysis

Values are mean±SE of the mean. Differences between values are examined using the nonparametric Mann-Whitney U test and are considered significant at a p value of <0.05.

This example demonstrates that TREM-1/TRIOPEP in free form is non-toxic and well tolerated in healthy mice at doses of up to at least 400 mg/kg. See FIG. 18.

### Example 15: Modulation of the TREM-1 Pathway in a Mouse Model of DSS-Induced Colitis and Colitis-Associated Tumorigenesis

In order to demonstrate that modulation of the TREM-1/DAP-12 signaling pathway using GF9 and the TREM-1-related TRIOPEP formulations are effective in inhibiting TREM-1-mediated cell activation, decreasing intestinal epithelial proliferation in dextran sulfate sodium (DSS)-induced colitis and ameliorating the development of inflammation and tumor within the colon through exerting anti-inflammatory effects, the experiments can be conducted analogously to those described in Zhou, et al. Int Immunopharmacol 2013, 17:155-161.

### GF9 and TREM-1-Related TRIOPEP Formulations

GF9, GF9-G, control peptide TREM-1-related TRIOPEP and TRIOPEP-A in free and SLP-bound form are synthesized as described herein.

### Animals and DSS-Induced Colitis and Colitis-Associated Tumorigenesis

C57BL/6 mice are purchased from Zhejiang Provincial Laboratories and (aged 8 to 12 weeks) maintained in a specific pathogen-free facility. Mice are treated with 7 days of 3.5% DSS (MP Biomedicals) in regular drinking water. To develop colitis-associated tumors, mice are first injected with 10 mg/kg azoxymethane (AOM) (Sigma-Aldrich) intraperitoneally (i.p.) followed 5 days later by a 5 day course of 2% DSS. Mice are then allowed to recover for 16 days with regular drinking water. The cycle of five days of 2% DSS followed by 16 days of regular drinking water is repeated twice. Mice are sacrificed 21 days after the last cycle of DSS for tumor counting. Colons are harvested, flushed of feces and longitudinally slit open to grossly count tumors with the aid of a magnifier and stereomicroscope.

### Treatments

Starting on day 0 (at the beginning of colitis induction), mice are treated once daily with GF9, GF9-G, TREM-1/TRIOPEP-A or TREM-1/TRIOPEP in free or SLP-bound form at various concentrations injected i.p. in 200 μl saline. To investigate the effects of blocking TREM-1 after induced inflammation, colitis is induced by 4% DSS for 4 days. After colitis induction, mice are administered with GF9, GF9-G, TREM-1/TRIOPEP-A or TREM-1/TRIOPEP in free or SLP-bound form for the next 5 days.

### Quantitative RT-PCR

Total RNA from colons is collected after colon tissue homogenization using the Trizol (Pierce). cDNA is synthesized using iScript (MBI) and then used in quantitative PCR reactions with SYBR Green using specific primers: TNF-alpha forward 5′-AGGCTGCCC CGACTACGT-3′ and reverse 5′-GACTTTCTCCTGGTATGAGATAGCAAA-3′; IFN-gamma forward 5′-CAGCAACAGCAAGGCGAAA-3′ and reverse 5′-CTGGACCTGTGGGTTGTT GAC-3′; IL-1beta forward 5′-TCGCTCAGGGTCACAAGAAA-3′ and reverse 5′-CATCAGAGGCAAGGAGGAAAAC-3′; IL-6 forward 5′-ACAAGTCGGAGGCTTAATTACACAT-3′ and reverse 5′-ATGTGTAATTAAGCCTCCGACTTGT-3′; IL-17 forward 5′-GCTCCAGAA GGCCCTCAGA-3′ and reverse 5′-AGCTTTCCCTCCGCATTGA-3′; macrophage inflammatory protein-2 (MIP-2) forward 5′-CACTCTCAAGGGCGGTCAA-3′ and reverse 5′-AGGCACATCAGGTACGATCCA-3′; 3-actin forward 5′-AGATTACTGCTCTGGCTC CTA-3′ and reverse 5′-CAAAGAAAGGGT GTAAAACG-3′. Relative expression levels of mRNA are normalized to β-actin. PCR products are separated on a 1.5% agarose gel and stained with ethidium bromide. Relative quantification of mRNA is performed by densitometry using QuantityOne software (e.g. Biorad Laboratories). Reactions are performed on the ABI 7900HT.

### ELISA

The serum levels of TNF-alpha, IL-1beta and IL-6 are measured using the specific ELISA kits (e.g. R&D Systems) following the manufacturer's instructions. All samples are ran in duplicate and analyzed on the same day.

### Evaluation of Inflammation

Colons are harvested from mice, flushed free of feces and jelly-rolled for formalin fixation and paraffin embedding. 5 m sections are used for hematoxylin and eosin staining. Histologic assessment is performed in a blinded fashion using a scoring system. A 3-4 point scale is used to denote the severity of inflammation (0=none, 1=mild, 2=moderate, and 3=severe), the level of involvement (0=none, 1=mucosa, 2=mucosa and submucosa and 3=transmural) and extent of epithelial/crypt damage (0=none, 1=basal 1/3, 2=basal 2/3, 3=crypt loss, and 4=crypt and surface epithelial destruction). Each parameter is then multiplied by a factor reflecting the percentage of the colon involved (0-25%, 26-50%, 51-75%, and 76-100%), and then summed to obtain the overall score. Assessment of colon weight after DSS treatment is performed by measuring the weight of colons (excluding the cecum) after removal of feces and normalizing by the length of colon in age- and sex-matched mice.

### Intestinal Permeability

Mice are fasted for 4 h with the exception of drinking water prior to the administration of 0.6 mg/kg FITC-dextran (4 kD, Sigma). Serum is collected 4 h later retro-orbitally, diluted 1:3 in PBS and the amount of fluorescence is measured using a fluorescent spectrophotometer with emission at 488 nm, and absorption at 525 nm.

### Intestinal Epithelial Proliferation

Mice are injected with 100 mg/kg BrdU (e.g. B.D. Pharmingen) i.p. 2.5 h prior to sacrifice at various time points after treatment with AOM/DSS. Colons are then dissected free, flushed free of feces, jelly-rolled, formalin-fixed, and paraffin-embedded. Sections are subsequently stained using the BrdU (e.g. BD Biosciences).

### Apoptosis.

Colon sections from formalin-fixed, paraffin-embedded tissues are assessed for apoptotic cells using the ApoAlert DNA fragmentation assay kit (e.g. Clontech).

### Statistics

Data are presented as mean±SEM. Survival curves is assessed by log-rank test. The tumor counts, intestinal permeability, cytokine measurements, proliferation and apoptosis levels between mice treated with GF9, GF9-G, TRIOPEP-A or TREM-1/TRIOPEP in free or SLP-bound form are compared using the Student's unpaired t-test. p<0.05 is considered statistically significant.

TREM-1 inhibition by treatment with GF9 and TREM-1/TRIOPEP but not GF9-G or TRIOPEP-A in free and SLP-bound form is anticipated to ameliorate the development of inflammation and tumor within the colon through exerting anti-inflammatory effects. In addition, this treatment is anticipated to decrease intestinal epithelial proliferation in DSS-induced colitis.

### Example 16: Synthesis and Modification of Paclitaxel-Conjugated Peptides in Free and SLP-Bound Form

This example demonstrates one embodiment of a synthesized trifunctional peptide compound containing PTX (PTX/TRIOPEP).

The first step is to synthesize the trifunctional compound comprising domains A and B where domain A is paclitaxel (PTX) bound to TREM-1 inhibitory peptide sequence GFLSKSLVF, whereas domain B is a 22 amino acids-long apolipoprotein A-I helix 6 peptide sequence with either unmodified or modified amino acid residue(s) (see TABLE 2). Although it is not necessary to understand the mechanism of an invention, it is believed that as an anticancer agent, PTX may exhibit not only its microtubule-stabilizing activity, but also its ability to stimulate release of anticancer cytokines from tumor-associated macrophages (TAMs) and functions to treat and/or prevent a cancer-related disease or condition, whereas a 22 amino acids-long apolipoprotein A-I helix 6 peptide sequence with either unmodified or modified amino acid residue(s) functions to assist in the self-assembly of SLP upon binding to lipid or lipid mixtures and to target the particles to cancer cells and/or TAMs, respectively.

In one embodiment, the trifunctional peptide compound comprises domains A and B where domain A is PTX is conjugated to TREM-1 inhibitory peptide sequence GFLSKSLVF, whereas domain B is a 22 amino acids-long apolipoprotein A-I helix 4 peptide sequence with either unmodified or sulfoxidized methionine residue (see TABLE 2).

In one embodiment, PTX is conjugated to the acetylated 31 amino acids-long sequence of TREM-1/TRIOPEP where the domain A comprises acetylated peptide sequence GFLSKSLVF whereas domain B comprises an apolipoprotein A-I helix 6 peptide sequence (i.e. PTX-Gly-Phe-Leu-Ser-Lys-Ser-Leu-Val-Phe-Pro-Leu-Gly-Glu-Glu-Met-Arg-Asp-Arg-Ala-Arg-Ala-His-Val-Asp-Ala-Leu-Arg-Thr-His-Leu-Ala-OH or PTX-GFLSKSLVFPLGEEMRDRARAHVDALRTHLA), hereafter referred to as a PTX/TREM-1-related “TRIOPEP” peptide compound or “PTX-TREM-1/TRIOPEP”.

Peptides can be synthesized or purchased from specialized companies (i.e., Sigma-Genosys, Woodlands, Tex., USA) with greater than 95% purity as assessed by HPLC. Peptide molecular mass can be checked by matrix-assisted laser desorption ionization mass spectrometry. The trifunctional peptide compounds containing conjugated PTX can be synthesized analogously as described in Lin et al. Chem Commun (Cambridge) 2013; 49:4968-4970 and disclosed in Castaigne, et al. U.S. Pat. No. 9,173,891.

### Synthesis of 4-(Pyridin-2-Yldisulfanyl) Butyric Acid

4-Bromobutyric acid (2 g, 12 mmol) and thiourea (0.96 g, 12.6 mmol) are dissolved in ethanol (50 mL) and refluxed at 90° C. for 4 h. After dropwise addition of a NaOH solution (4.8 g in 5:1 H2O/ethanol), the mixture is refluxed for another 16 h and then cooled to room temperature. The white precipitate is collected and redissolved in water (40 mL). 4 M HCl is used to adjust the solution pH to 5, and the product is extracted into diethyl ether. The organic phase is dried over anhydrous sodium sulfate to give 4-sulfanylbutyric acid as a colorless oil (310 mg, 15%), which is used in the next step without further purification. 4-sulfanylbutyric acid (105 mg, 0.87 mmol) and 2-aldrithiol (440 mg, 2.0 mmol, 2.3 eq) are dissolved in MeOH (1.3 mL) and stirred for 3 h. The solution is purified by RP-HPLC (5% to 95% of acetonitrile in water with 0.1% TFA over 45 min), combining product fractions and removing solvents to give 4-(pyridin-2-yldisulfanyl) butyric acid as an oil (118 mg, 59%).

### Paclitaxel C2′ Ester Synthesis

Paclitaxel (186 mg, 0.22 mmol), 4-(pyridin-2-yldisulfanyl)butyric acid (100 mg, 0.44 mmol), N,N′-diisopropylcarbodiimide (DIC) (68 μL, 0.44 mol), and 4-dimethylaminopyridine (DMAP) (26.7 mg, 0.22 mmol) are added into an oven dried flask equipped with a stirrer bar, evacuated and refilled with nitrogen three times to remove air, then dissolved in anhydrous acetonitrile (12.7 mL). The reaction is allowed to stir in the dark at room temperature for 48 h. The solvents are removed under vacuum and the residue is dissolved in chloroform and purified by flash chromatography (3:2 EtOAc/hexane), to give the product as a white solid (108 mg, 47%).

### Synthesis of PTX-TREM-1/TRIOPEP in Free and SLP-Bound Form

GFLSKSLVFPLGEEMRDRARAHVDALRTHLA (89.8 mg, 25.7 umol) and paclitaxel C2′ ester (54.7 mg, 51.4 umol) are added to an oven dried flask equipped with a stirrer bar and evacuated and filled with nitrogen three times to remove the air. The reagents are then dissolved in anhydrous dimethyl formamide DMF (5 mL). The solution is allowed to stir for 16 h, before purification by RP-HPLC (30% to 95% acetonitrile in water with 0.1% TFA over 45 min). Product fractions are combined and lyophilized to give a PTX-TREM-1/TRIOPEP as a white powder. Discoidal and spherical PTX-TREM-1/TRIOPEP-containing SLP are prepared, purified and characterized using the methods and procedures described herein in the Example 2.

### Example 17: Use of PTX-TREM-1/TRIOPEP in Experimental Cancer

In order to demonstrate the anticancer activity of PTX-TREM-1/TRIOPEP, the experiments can be conducted analogously to those disclosed herein and described in Lin et al. Chem Commun (Cambridge) 2013; 49:4968-4970; Sigalov. Int Immunopharmacol 2014, 21:208-219; Shen and Sigalov. Mol Pharm 2017, 14:4572-4582; and disclosed in Castaigne, et al. U.S. Pat. No. 9,173,891.

### Cytotoxicity

The methyl thiazol tetrazolium (MTT) assay can be used to assess the cytotoxic effect of the PTX-TREM-1/TRIOPEP formulations in free or SLP-bound form on cancer cells. The PTX-TREM-1/TRIOPEP formulations may contain either unmodified or modified amino acid residue(s). Briefly, cells are plated in 96-well plates (5000 cells/well) in their respective media. Next day, the monolayers are washed with PBS (pH 7.4) twice, and then incubated at 37° C. for 24 h with the PTX-TREM-1/TRIOPEP formulations in free or SLP-bound form in serum-free media. The following day, 25 μl of MTT (1 mg/ml) is added to each well and incubated for 3 h at 37° C. Plates are centrifuged at 1200 rpm for 5 min. The medium is removed, the precipitates are dissolved in 200 μl of DMSO and the samples are read at 540 nm in a microtiter plate reader.

### Animal Toxicity

Female C57BL6 mice (6-8 weeks, 18-21 g) can be used in toxicity studies of PTX-TREM-1/TRIOPEP formulations in free or SLP-bound form. PTX-TREM-1/TRIOPEP formulations may contain either unmodified or oxidized methionine residue. Groups of six mice each receives injections of 1.5 ml of PBS via the intraperitoneal route, containing respective doses of 30 mg/kg and 40 mg/kg of Taxol®, 40 mg/kg and 70 mg/kg of Abraxane® and different doses of PTX-TREM-1/TRIOPEP in free or SLP-bound form. The injections are administered on days 1, 2 and 3. A control group is injected with the vehicle. The weights and the health of the mice are monitored for 30 days. Weight measurements are performed once a day for the first 7 days and twice a week for the remaining monitoring period.

### Screening for PTX-TREM-1/TRIOPEP Incorporation

Cultured cells are incubated with PTX-TREM-1/TRIOPEP formulations in free or SLP-bound form, labeled with ¹⁴C-PTX. Subsequent to the incubation period, cells are trypsinized and the radioactivity of the lysate is determined to measure the extent of incorporation of the PTX into the cells.

### Tumor Suppression

Tumor suppression studies using PTX-TREM-1/TRIOPEP formulations in free or SLP-bound form can be performed in animal models of cancer similarly as described herein (see e.g., the examples 7 and 8). Female 6-8 week old NU/J mice can be obtained from the Jackson Laboratory (Bar Harbor, Me.) Human cancer cell lines including but not limited to human carcinoma, human pancreas or human breast cancer cell lines can be obtained from ATCC. Tumor cells in culture can be harvested and resuspended in a 1:1 ratio of RPMI 1640 and Matrigel (BD Biosciences, San Jose, Calif.). Human cancer xenografts are established by injecting subcutaneously into the right flanks certain amounts of viable cells per mouse. Tumor volumes are calculated with caliper measurements using the formula V=π/6 (length×width×width). When tumor grows to approximately 125 mm³ (100-150 mm³), animals are pair-matched by tumor size into treatment and control groups. Either PTX (TAXOL®; 30 mg/kg PTX) or PTX-TREM-1/TRIOPEP formulations in free (60 mg/kg PTX) or SLP-bound (30 mg/kg PTX) are intravenously administered to the animals via tail vein. Clinical observations, body weights and tumor volume measurements are made twice a week once tumors become measureable. It should be noted that TAXOL® is formulated with a detergent Cremophor that in itself is cytotoxic and is also the source of numerous side effects during chemotherapy. The Cremophor content of TAXOL® is about 80× that of paclitaxel per ml.

TREM-1 inhibition by treatment with PTX-TREM-1/TRIOPEP in free and SLP-bound form is anticipated to have a significantly higher anticancer activity in terms of tumor inhibition and survival rate improvement as compared to PTX. In addition, this treatment is anticipated to be substantially better tolerated by cancer mice as compared to PTX.

### Example 18: Modulation of the TREM-1 Pathway in Experimental Arthritis

In order to demonstrate that GF9 and TREM-1-related TRIOPEP formulations are effective in inhibiting TREM-1-mediated cell activation and protecting against bone and cartilage damage in animal models of rheumatoid arthritis (RA), the experiments were conducted as described in Shen and Sigalov. J Cell Mol Med 2017, 21:2524-2534.

### Chemicals, Lipids and Cells

Sodium cholate, cholesteryl oleate and other chemicals were purchased from Sigma-Aldrich Company (St. Louis, Mo., USA). 1,2-dimyristoyl-sn-glycero-3-phosphocholine (DMPC), 1,2-dimyristoyl-sn-glycero-3-phospho-(10-rac-glycerol) (DMPG), 1-palmitoyl-2-oleoyl-sn-glycero-3-phosphocholine (POPC), 1-palmitoyl-2-oleoyl-sn-glycero-3-phospho-(10-rac-glycerol) (POPG), 1,2-dimyristoyl-sn-glycero-3-phosphoethanolamine-N-(lissamine rhodamine B sulfonyl) (Rho B-PE) and cholesterol were purchased from Avanti Polar Lipids (Alabaster, Ala., USA). The murine macrophage cell line J774A.1 was obtained from the American Type Culture Collection (ATCC, Manassas, Va., USA).

### Peptide Synthesis

GF9 and two 31-mer methionine-sulfoxidized peptides, GFLSKSLVFPYLDDFQKKWQEEM(O)ELYRQKVE (GE31) and GFLSKSLVFPLGEEM(O)RDRARAHVDALRTHLA (GA31) were ordered from American Peptide Company (Sunnyvale, Calif., USA). All peptides were purified by reversed-phase high-performance liquid chromatography (RP-HPLC), and their purity was confirmed by amino acid analysis and mass spectrometry.

### Synthetic Lipopeptide Particles (SLP)

Discoidal SLP (dSLP) complexes that contain GF9 or an equimolar mixture of TREM-1/TRIOPEP peptides GA31 and GE31 (TREM-1/TRIOPEP) were synthesized as described in Shen and Sigalov. J Cell Mol Med 2017, 21:2524-2534 and herein (see the Example 2). The molar ratio was 65:25:1:190 corresponding to DMPC:DMPG:GA/E31:sodium cholate for GA/E31-dHDL that contain an equimolar mixture of oxidized TREM-1/TRIOPEP peptides GA31 and GE31. Spherical SLP (sSLP) complexes that contain an equimolar mixture of GA31 and GE31 (TREM-1/TRIOPEP-sSLP) were synthesized as described in Shen and Sigalov. J Cell Mol Med 2017, 21:2524-2534 and herein (see the Example 2). The molar ratio was 125:6:2:1:210 corresponding to POPC:cholesterol:cholesteryl oleate:GA/E31-I:sodium cholate for TREM-1/TRIOPEP-sSLP that contain an equimolar mixture of oxidized peptides GA31 and GE31. All obtained SLP formulations were purified and characterized as described in Shen and Sigalov. J Cell Mol Med 2017, 21:2524-2534 and herein (see the Example 2).

### Animals

All animal experiments were performed in strict accordance with the recommendations in the Guide for the Care and Use of Laboratory Animals of the National Institutes of Health (NIH) and in the United States Department of Agriculture (USDA) Animal Welfare Act (9 CFR, Parts 1, 2, and 3).

### Collagen-Induced Arthritis (CIA) Model

Animal studies were performed by Bolder BioPATH (Boulder, Colo., USA). CIA was induced in male 6- to 7-week-old DBA/1 mice by immunization with bovine type II collagen. Briefly, mice were injected intradermally with 100 μl of Freund's complete adjuvant containing 250 μg of bovine type II collagen (2 mg/ml final concentration) at the base of the tail on day 0 and again on day 21. On day 24, mice were randomized by body weight into treatment groups. At enrolment on day 24, the mean mouse weight was 20 g. Arthritis onset occurred on days 26-38. Starting day 24, mice were injected i.p. intraperitoneally daily for 14 consecutive days with GF9, GF9-dSLP, GF9-sSLP, TREM-1/TRIOPEP-dSLP (dose equivalent to 5 mg of GF9/kg), TREM-1/TRIOPEP-sSLP (dose equivalent to 5 mg of GF9/kg) or with PBS. Mice were weighed on study days 24, 26, 28, 30, 32, 34, 36 and 38 (prior to necropsy). Daily clinical scores were given on a scale of 0-5 for each of the paws on days 24-38. On day 38, mice were killed for necropsy.

### Histology Assessment of Joints

At the end of study, fore paws, hind paws and knees were harvested, fixed in 10% neutral buffered formalin for 1-2 days, and then decalcified in 5% formic acid for 4-5 days before standard processing for paraffin embedding. Sections (8 μm) were cut and stained with toluidine blue (T blue). Hind paws, fore paws and knees were embedded and sectioned in the frontal plane. Six joints from each animal were processed for histopathological evaluation. The joints were then assessed using 0-5 scale for inflammation, pannus formation, cartilage damage, bone resorption and periosteal new bone formation. A summed histopathology score (sum of five parameters, 0-25 scale) was also determined.

### Cytokine Detection

Plasma was collected on days 24, 30 and 38, and cytokines were analysed by Quantibody Mouse Cytokine Array Q1 kits (RayBiotech, Norcross, Ga., USA) according to the manufacturer's instructions.

### Statistical Analysis

All statistical analyses were performed with GraphPad Prism 6.0 software (GraphPad, La Jolla, Calif., USA). Results are expressed as the mean±SEM. Statistical differences were analyzed using analysis of variance with Bonferroni adjustment. P values less than 0.05 were considered significant.

This example demonstrates that GF9 or TREM-1/TRIOPEP in free or SLP-bound form ameliorate CIA and protect against bone and cartilage damage. This therapeutic effect is accompanied by a reduction in the plasma levels of macrophage colony-stimulating factor and pro-inflammatory cytokines such as TNF-alpha, interleukin (IL)-1 and IL-6. This example further demonstrates that GF9, GF9-SLP, TREM-1/TRIOPEP-SLP formulations are non-toxic and well-tolerable by arthritic mice. See FIG. 17A-B.

### Example 19: Modulation of the TREM-1 Pathway in Experimental Retinopathy

In order to demonstrate that GF9 and TREM-1-related TRIOPEP formulations are effective in inhibiting TREM-1-mediated cell activation and reducing pathological retinal neovascularization (RNV), the experiments were conducted as described in Rojas, et al. Biochim Biophys Acta 2018, 1864:2761-2768, herein incorporated by reference in it's entirety.

### Synthetic Lipopeptide Particles (SLP)

Spherical SLP that contain GF9 or an equimolar mixture of GA31 and GE31 (TREM-1/TRIOPEP-sSLP) were synthesized using the sodium cholate dialysis procedure, purified and characterized as described herein and in Shen and Sigalov. Mol Pharm 2017, 14:4572-4582 and Shen and Sigalov. J Cell Mol Med 2017, 21:2524-2534. In a subset of experiments, 1,2-dimyristoyl-sn-glycero-3-phosphoethanolamine-N-(lissamine rhodamine B sulfonyl) was added to reaction mixtures to prepare rhodamine B (rho-B)-labeled rho B-labeled TREM-1/TRIOPEP-sSLP as described in Shen and Sigalov. Mol Pharm 2017, 14:4572-4582 and Shen and Sigalov. J Cell Mol Med 2017, 21:2524-2534.

### In Vitro Macrophage Uptake.

BALB/c murine macrophage J774A.1 cells were obtained from ATCC (Manassas, Va.) and cultured according to manufacturer's instructions at 37° C. in 6-well tissue culture plates containing glass coverslips until reaching about 50% confluency. Then, cells were incubated for 6 h at 37° C. either with rho B-labeled GF9-SLP that contained Dylight labeled GF9 or TREM-1/TRIOPEP-sSLP that contained Dylight 488-labeled GE31. In colocalization experiments, TREM-1 staining was performed using an Alexa 647-labeled rat anti-mouse TREM-1 antibody (Bio-Rad, Hercules, Calif.) as described in Shen and Sigalov. J Cell Mol Med 2017, 21:2524-2534. Coverslips were mounted using Prolong Gold anti-fade DAPI (4′,6-diamidino-2-phenylindole) mounting medium and photographed using an Olympus BX60 fluorescence microscope. Confocal imaging was performed using a Leica TCS SP5 II laser scanning confocal microscope as described in Shen and Sigalov. J Cell Mol Med 2017, 21:2524-2534.

### Mouse Model of Oxygen-Induced Retinopathy (OIR)

This study was carried out in strict accordance with the recommendations in the Guide for the Care and Use of Laboratory Animals of the National Institutes of Health and in the United States Department of Agriculture (USDA) Animal Welfare Act (9 CFR, Parts 1, 2, and 3). animals were treated according to the ARVO Statement for the Use of Animals in Ophthalmic and Vision Research.

Litters of C57BL/6J (Jackson Laboratory, Bar Harbor, Me.) neonatal mice and nursing dams were exposed to a hyperoxia environment (75% oxygen) from postnatal day 7 (P7) to P12 and returned to normoxia until P17. The hyperoxia exposure causes degeneration of the immature retinal vessels. This results in severe hypoxia upon return to the normoxia environment which leads to vitreoretinal neovascularization. Beginning on P7, mice were treated until day P17 by daily i.p. injections of GF9, GF9-SLP, TREM-1/TRIOPEP-sSLP or vehicle (phosphate-buffered saline, pH 7.4; PBS). In a subset of experiments, rho B-labeled GF9-sSLP and TREM-1/TRIOPEP-sSLP were used to confirm the ability of these particles to cross the BRB. In another subset of experiments, rho B-labeled Gd-containing sSLP were used to confirm the ability of these targeted SLP to cross the BRB in other species (rats and rabbits). In another subset of experiments, neonatal mice and nursing dams were not subjected to a hyperoxia environment and reared in room air (RA). At P17, all mice were humanely sacrificed and their retinas were collected.

### Immunofluorescence Staining

Treatment effects on vaso-obliteration and pathological angiogenesis were assessed by morphometric analysis of the avascular and neovascularization areas in retinal flat mounts after labeling with isolectin B₄ as described in Patel, et al. Am J Pathol 2014, 184:3040-3051. Immunofluorescence analysis (IFA) of the retina flat mounts was performed to assess the effects of the TREM-1-targeting treatments on the distribution of TREM-1, M-CSF and markers for inflammatory cells (CD45) and activated macrophage/microglial cells (Iba-1) in relation to RNV. Retinal frozen sections from pups kept in RA and from the OIR pups were fixed in 4% paraformaldehyde for 15 min (or in cold acetone at −20° C. for 30 min), washed 3 times with PBS, and blocked with a solution containing 0.3% Triton X and 3% normal goat serum (NGS) for 30 min. Then, the samples were reacted with a rat anti-mouse TREM-1 antibody (Abcam, Cambridge, Mass.), rabbit polyclonal anti-mouse M-CSF antibodies (Abcam, Cambridge, Mass.), rabbit polyclonal anti-mouse CD45 antibodies (Santa Cruz Biotechnology, Dallas, Tex.), a rabbit anti-mouse Iba-1 antibody (Wako Chemical USA, Inc.), and kept at 4° C. overnight. Then, the samples were washed 3 times with PBS and stained with a donkey-anti-rat Oregon green antibody for TREM-1, a goat anti-rabbit Texas red antibody for CD45 and Iba-1 or a donkey anti-rabbit Texas red antibody for M-CSF (Invitrogen, Waltham, Mass.). After washing 3 times with PBS, the images were captured with a 20× lens using a Zeiss Axioplan2 fluorescence microscope (Carl Zeiss Meditec, Inc., Dublin, Calif.). Intravitreal neovascular formation and avascular area were measured as described in Connor, et al. Nat Protoc 2009, 4:1565-1573.

### Western Blot Analysis

Retina samples from OIR-treated and RA control pups were homogenized in the modified RIPA buffer (20 mM Tris-HCl, 2.5 mM EDTA, 50 mM NaF, 10 mM Na₄P₂O₇, 1% Triton X-100, 0.1% sodium dodecyl sulfate, 1% sodium deoxycholate, 1 mM phenyl methyl sulfonyl fluoride, pH 7.4). Samples containing equal amounts of protein were separated by 12% sodium dodecyl sulfate polyacrylamide gel electrophoresis, transferred to nitrocellulose membrane, and reacted for 24 hrs with monoclonal rat anti-mouse TREM-1 or polyclonal rabbit M-CSF antibodies (Abcam, Cambridge, Mass.) in 5% milk, followed by incubation with corresponding horseradish peroxidase-linked secondary antibodies (GE Healthcare Bio-Science Corp., Piscataway, N.J.). Bands were quantified by densitometry, and the data were analyzed using ImageJ software and normalized to loading control. Equal loading was verified by stripping the membranes and reprobing them with a monoclonal antibody against β-actin (Sigma-Aldrich, St Louis, Mo.).

### Statistical Analysis

Group differences were compared by one way ANOVA followed with a post hoc test for multiple comparisons. Values are represented as the means±standard error of the means (SEM). Results were considered statistically significant when P≤0.05.

This example demonstrates that GF9 and TREM-1/TRIOPEP in free and SLP-bound form significantly (up to 95%) reduce pathological RNV in a mouse model of retinopathy. It further that demonstrates that GF9 and TREM-1/TRIOPEP in free and SLP-bound form are non-toxic and well-tolerated in mouse litters. TREM-1 inhibition substantially downregulates retinal protein levels of TREM-1 and M-CSF (CSF-1) suggesting that TREM-1-dependent suppression of pathological angiogenesis involves M-CSF (CSF-1). This example further demonstrates that sSLP, GF9, GF9-SLP and TREM-1/TRIOPEP-sSLP pass the blood-retinal barrier (BRB) and blood-brain barrier (BBB). See FIGS. 18A-D-19.

### Example 20: Modulation of the TREM-1 Pathway in Experimental Alcoholic Liver Disease (ALD)

In order to demonstrate that TREM-1-related TRIOPEP formulations are effective in inhibiting TREM-1-mediated cell activation and ameliorating ALD, the experiments were conducted in the Lieber DeCarli ALD mouse model as described in Tornai et al. Hepatol Commun 2019,3:99-115, and Petrasek, et al. J Clin Invest 2012, 122:3476-3489.

### Reagents and Cells

The murine macrophage J774A.1 cells were purchased from ATCC. Cytochalasin D was purchased from MP Biomedicals (Solon, Ohio, USA). Blocker of lipid transport 1 (BLT-1) was purchased from Calbiochem (Torrey Pines, Calif., USA). Sodium cholate, cholesteryl oleate, fucoidan and other chemicals were purchased from Sigma-Aldrich (St. Louis, Mo., USA). 1-palmitoyl-2-oleoyl-sn-glycero-3-phosphocholine (POPC), 1,2-dimyristoyl-sn-glycero-3-phosphoethanolamine-N-(lissamine rhodamine B sulfonyl) (rho B-PE) and cholesterol were purchased from Avanti Polar Lipids (Alabaster, Ala., USA).

### Peptide Synthesis

The following synthetic peptides were ordered from Bachem (Torrance, Calif., USA): one 9-mer peptide GFLSKSLVF (human TREM-1213-221, GF9), two 22-mer methionine sulfoxidized peptides PYLDDFQKKWQEEM(O)ELYRQKVE (H4) and PLGEEM(O)RDRARAHVDALRTHLA (H6) that correspond to human apo A-I helices 4 (apo A-I123-144) and 6 (apo A-I167-188), respectively, and two 31-mer methionine sulfoxidized peptides, GFLSKSLVFPYLDDFQKKWQEEM(O)ELYRQKVE (GE31) and GFLSKSLVFPLGEEM(O)RDRARAHVDALRTHLA (GA31).

### Synthetic Lipopeptide Particles (SLP)

SLP of spherical morphology that contained either GF9 and an equimolar mixture of PE22 and PA22 (GF9-sSLP) or an equimolar mixture of GA31 and GE31 (TREM-1/TRIOPEP-sSLP) were synthesized using the sodium cholate dialysis procedure, purified and characterized as previously described herein and in Shen and Sigalov. Mol Pharm 2017, 14:4572-4582 and Shen and Sigalov. J Cell Mol Med 2017, 21:2524-2534. For GF9-sSLP, the initial molar ratio was 125:6:2:3:1:210 corresponding to POPC:cholesterol:cholesteryl oleate:GF9:apo A-I:sodium cholate, respectively, where apo A-I is an equimolar mixture of PE22 and PA22. For TREM-1/TRIOPEP-sSLP, the initial molar ration was 125:6:2:1:210 corresponding to POPC:cholesterol:cholesteryl oleate:GA/E31:sodium cholate, where GA/E31 is an equimolar mixture of GA31 and GE31 peptides.

### In Vitro Macrophage Uptake

BALB/c murine macrophage J774A.1 cells (ATCC, Manassas, Va., USA) were cultured at 37° C. with 5% CO2 in Dulbecco's Modification of Eagle's Medium, DMEM (Cellgro Mediatech, Manassas, Va., USA) with 2 mM glutamine, 100 U ml-1 penicillin, 0.1 mg/ml streptomycin and 10% heat inactivated fetal bovine serum (Cellgro Mediatech, Manassas, Va., USA) and grown to approximately 90% confluency in 12 well tissue culture plates (Corning Costar, Corning, N.Y., USA). After reaching target confluency, cells were incubated for 1 h in medium with or without fucoidan (400 μg/mL), BLT-1 (10 μM) or cytochalasin D (40 μM), Cells were subsequently incubated for 4 h and 22 h at 37° C. in medium containing 2 μM of rho B-labeled GF9-sSLP or TREM-1/TRIOPEP-sSLP (as calculated for rho B). Cells were washed twice using PBS and lysed using Passive Lysis Buffer (Promega, Madison, Wis., USA). Rho B fluorescence was measured in the lysates with 544 nm excitation and 590 nm emission filters using a Fluoroscan Ascent CF fluorescence microplate reader (Thermo Labsystems, Vantaa, Finland). The protein concentrations in the lysates were measured using Bradford Reagent (Sigma-Aldrich, St. Louis, Mo., USA) and a MRX microplate reader (Dynex Technologies, Chantilly, Va., USA) according to the manufacturer's recommended protocol.

### Animals

C57BL/6 female mice (10- to 12-week-old) were purchased from The Jackson Laboratory (Bar Harbor, Me., USA) and housed at the University of Massachusetts Medical School (UMMS) animal facility. All animals received humane care in accord with protocols approved by the UMMS Institutional Animal Use and Care Committee. Mice (n=6-9/group) were acclimated to a Lieber-DeCarli liquid diet of 5% ethanol (vol/vol) over a period of 1 week, then maintained on the 5% diet for 4 weeks. Pair-fed control mice were fed a calorie-matched dextran-maltose diet. All animals had unrestricted access to water throughout the entire experimental period. In treated groups, mice were i.p. treated 5 days/week with vehicle (empty sSLP) or the TREM-1 inhibitory formulations GF9-sSLP (2.5 mg of GF9/kg) or TREM-1/TRIOPEP-sSLP (5 mg equivalent of GF9/kg) (SignaBlok, MA, USA), from the first day on a 5% ethanol diet. At the end of all animal experiments, cheek blood samples were collected in serum collection tubes (BD Biosciences, San Jose, Calif., USA) and processed within an hour. After blood collections, mice were euthanized, and liver samples were harvested and stored at −80° C. until further analysis.

Total Protein Isolation from Liver

Total protein was extracted from liver samples using RIPA buffer (Boston Bio-products Cat. #BP-115) supplemented with protease inhibitor cocktail tablets (Roche Cat. #11836153001) and Phospho Stop phosphatase inhibitor (Roche Cat. #04906837001). Cell debris were then removed from cell lysates by 10 minutes centrifugation at 2000 rpm.

### Biochemical Assays and Cytokines

Serum alanine aminotransferase (ALT) levels were determined by kinetic method using commercially available reagents from Teco Diagnostics (Anaheim, Calif., USA). Liver triglycerides were extracted using a 5% NP-40 lysis solution buffer and quantified using a commercially available kit (Wako Chemicals, Richmond, Va., USA) followed normalization to protein amount analyzed by Pierce BCA protein assay (Thermo Scientific, Rockford, Ill., USA). Cytokine levels were measured in serum samples and whole liver lysates diluted in assay diluent following the manufacturer's instructions. Specific anti-mouse ELISA kits were used for the quantification of MCP-1, TNFα (BioLegend Inc., San Diego, Calif., USA) and IL-1β (R&D Systems, Minneapolis, Minn., USA) levels. For normalization, the total protein concentration of the whole liver lysate was determined using Pierce BCA protein assay.

### Western Blot Analysis

Whole liver proteins were boiled in Laemmli's buffer. The samples were resolved in 10% SDS-PAGE gel under reducing conditions using Tris-glycine buffer system and resolved proteins transferred onto a nitrocellulose membrane. SYK proteins were detected by specific primary antibodies (SYK: 2712—Cell Signaling and phospho-SYKY525/526: ab58575—Abcam) followed by an appropriate secondary HRP-conjugated IgG antibody from Santa Cruz Biotechnology. β-actin, detected by an ab49900 antibody (Abcam), was used as a loading control. The specific immunoreactive bands of interest were visualized by chemiluminescence (Bio-Rad) using the Fujifilm LAS-4000 luminescent image analyzer.

### RNA Extraction and Quantitative Real-Time PCR Analysis

Total RNA was extracted using the Qiagen RNeasy kit (Qiagen) according to the manufacturer's instructions with on-column DNase treatment. RNA was quantified using a Nanodrop 2000 spectrophotometer (Thermo Scientific) and cDNA synthesis was performed using the iScript Reverse Transcription Supermix (Bio-Rad Laboratories) and 1 μg total RNA. Real-time quantitative PCR was performed using Bio-Rad iTaq Universal SYBR Green Supermix (Bio-Rad Laboratories) and a CFX96 real-time detection system (Bio-Rad Laboratories). Relative gene expression was calculated by the comparative ΔΔCt method. The expression level of target genes was normalized to the house-keeping gene, 18S rRNA, in each sample and the fold-change in the target gene expression between experimental groups was expressed as a ratio. Primers were synthesized by IDT, Inc. and the sequences are listed in Table 3A.

### Liver Histopathology

Sections of formalin-fixed, paraffin-embedded liver specimens from mice were stained with Hematoxylin/Eosin (H&E) or F4/80 (ThermoFisher, Cat #MF48000), MPO (Abcam Cat #ab9535) antibodies for immunohistochemistry, the fresh frozen samples were stained with Oil-Red-O at the UMMS DERC histology core facility.

### Statistical Analysis

All statistical analyses were performed using GraphPad Prism 7.02 (GraphPad Software Inc.). Significance levels were determined using one way analysis of variance (ANOVA) followed by a post hoc test for multiple comparisons. Data are shown as mean±SEM and differences were considered statistically significant when P≤0.05. Significance levels were showed using the following symbols: *, 0.05≥P≥0.01; ** 0.01≥P≥0.001; ***, 0.001≥P≥0.0001; ****, P≤0.0001.

This example demonstrates that TREM-1/TRIOPEP in SLP-bound form significantly reduced serum ALT and cytokine protein levels in a mouse model of ALD. It further that demonstrates that TREM-1/TRIOPEP in SLP-bound form are non-toxic and well-tolerated in mice with ALD. TREM-1/TRIOPEP significantly inhibits macrophage (F4/80, CD68) and neutrophil (lymphocyte antigen 6 complex locus G6D and myeloperoxidase, Ly6G and MPO, respectively) markers and proinflammatory cytokines monocyte chemoattractant protein-1, tumor necrosis factor-α, interleukin-1β and macrophage inflammatory protein-1α (MCP-1, TNF-α, IL-1β, MIP-1α, respectively) at the mRNA level as compared to the sSLP vehicle. This example further demonstrates that TREM-1/TRIOPEP-sSLP formulations ameliorates liver steatosis and early fibrosis markers (α-smooth muscle actin, αSMA, and pro-collagen1α) on the mRNA level in alcohol-fed mice. See FIG. 20.

### Example 7A: Immunofluorescence Analysis of TREM-1/TRIOPEP G-KV21 in the Cell Membrane

Immunofluorescence analysis of TREM-1/TRIOPEP G-KV21 in the cell membrane was performed using the standard, well-known in the art methods as described in Shen and Sigalov J Cell Mol Med 2017, 21:2524-2534.

BALB/c murine macrophage J774A.1 cells were grown at 37° C. in six-well tissue culture plates containing glass coverslips. After reaching target confluency of approximately 50%, cells were incubated for 6 h at 37° C. with Dylight 488-labeled G-KV21 that was pre-incubated with HDL. TREM-1 staining was performed using an Alexa 647-labeled rat anti-mouse TREM-1 antibody (Bio-Rad, Hercules, Calif.). ProLong Gold Antifade DAPI (4′,6′-diamidino-2-phenylindole) mounting medium was used to mount coverslips, and the slides were photographed using an Olympus BX60 fluorescence microscope. Confocal imaging was performed with a Leica TCS SP5 II laser scanning confocal microscope.

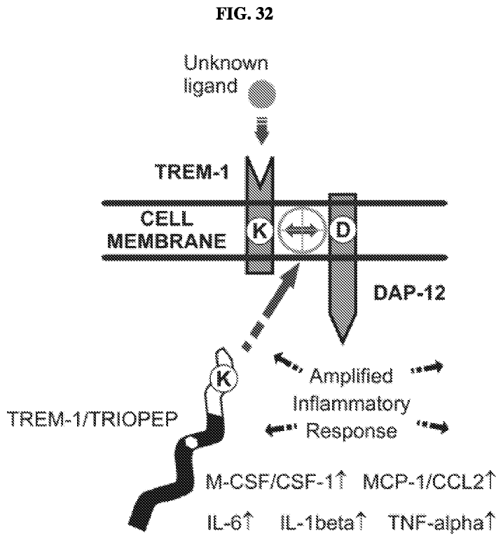

This example demonstrates that upon endocytosis by macrophages, TREM-1/TRIOPEP G-KV21 is released by native lipoproteins, self-inserts into the cell membrane and colocalizes with TREM-1. See FIG. 32.

### Example 8A: In Vitro Cytokine Release

In vitro studies of cytokine release by lipopolysaccharide (LPS)-stimulated macrophages in the presence of GF9, G-TE21, G-HV21 and G-KV21 either pre-incubated or not with HDL were performed using the standard, well-known in the art methods as described in Sigalov. Int Immunopharmacol 2014, 21:208-219.

The BALB/c murine macrophage cell line J774A.1 (ATCC TIB-67) were obtained from the American Type Culture Collection (ATCC, Manassas, Va.). Macrophages were cultured in 48-well plates (Corning, Cambridge, Mass.) for 24 h at 37° C. in the presence of LPS (1 μg/ml, Escherichia coli 055:B5, Sigma) in combination with 10 ng/ml peptides either pre-incubated or not with HDL. Cell-free supernatants were harvested and stored at −20° C. for later cytokine quantification. TNF-alpha, IL-6, and IL-1beta were assayed using commercial ELISA kits (Pierce Biotechnology, Thermo Scientific, Rockford, Ill.) according to the recommendations of the manufacturer. Results were represented as the mean±S.D. of three independent experiments. Statistical significances in in vitro macrophage uptake assay were determined by two-tailed Student's t test.

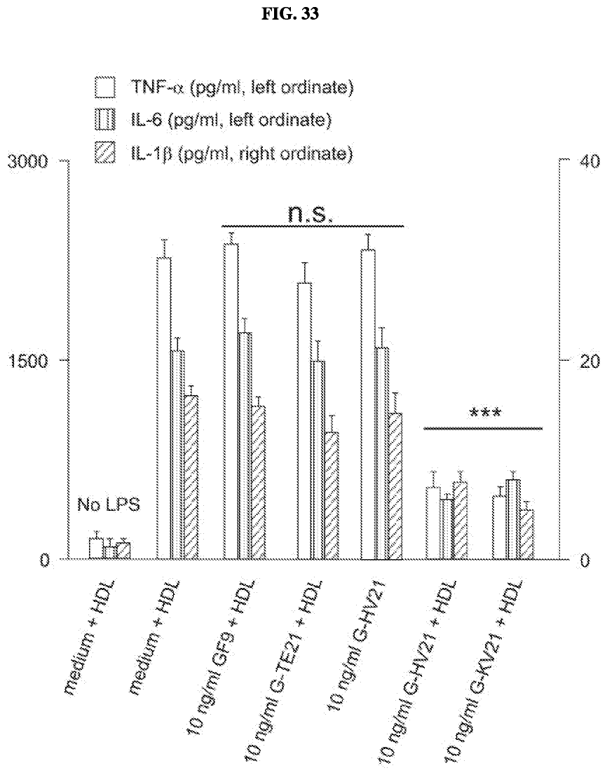

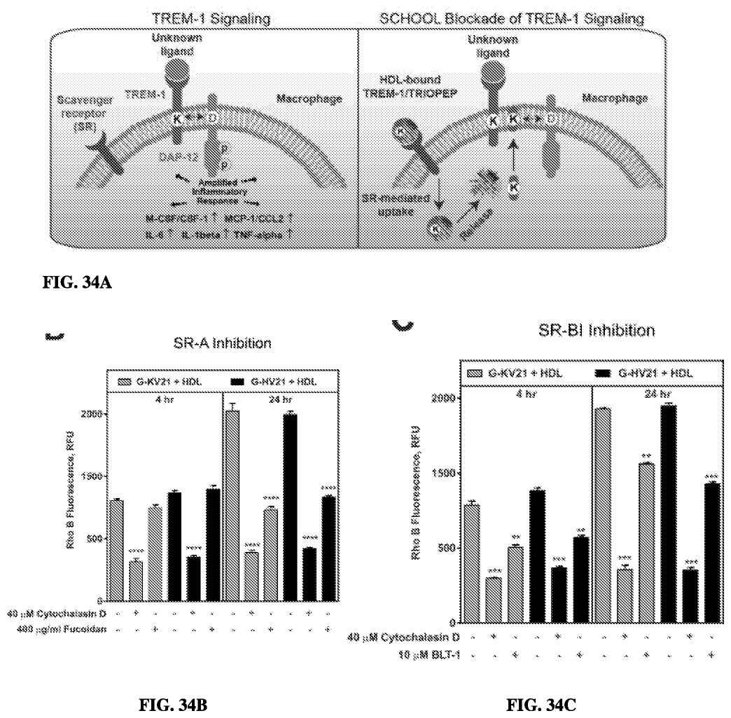

This example demonstrates that after pre-incubation with HDL, G-HV21 and G-KV21 but not GF9 or G-TE21 inhibit production of cytokines by LPS-stimulated macrophages. This example further demonstrates that after pre-incubation with HHDL, G-TE21 does not affect on cytokine release by LPS-stimulated macrophages. See FIG. 33.

### Example 9A: Mouse Model of LPS-Induced Endotoxemia and In Vivo Survival and Cytokine Release Studies

Animal survival studies and studies of in vivo cytokine release were performed in a mouse model of LPS-induced septic shock using the standard, well known in the art methods as described in Sigalov. Int Immunopharmacol 2014, 21:208-219.

Naïve, female C57BL/6 mice at 8 to 10 weeks of age (18 to 21 g) from the Jackson Laboratory were randomly grouped (10 mice per group) and i.p. injected with vehicle or the indicated doses of dexamethasone (DEX), GF9, G-TE21, G-HV21 and G-KV21. One hour later, mice received i.p. injection of 30 mg/kg LPS from E. coli 055:B5 (Sigma). In some experiments, all formulations were i.p. administered 1 and 3 h after LPS injection. The viability of mice was examined hourly. Body weights were measured daily. In all of the animal experiments, blood samples were collected via a sub-mandibular (cheek) bleed at 90 min after administration of LPS. Statistical analysis of survival curves was performed by the Kaplan-Meier test. Comparisons were made using two-tailed Student's t test. The production of cytokines in serum was measured by a standard sandwich cytokine ELISA procedure using TNF-alpha, IL-1beta and IL-6 ELISA kits (Pierce Biotechnology, Thermo Scientific, Rockford, Ill.) according to the instructions of the manufacturer. Statistical significances in cytokine analysis ELISA data were determined by two-tailed Student's t test.

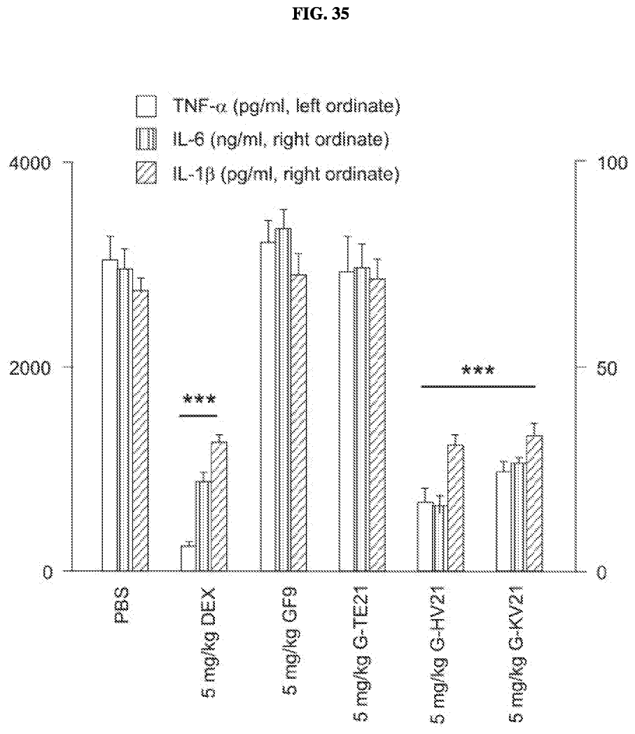

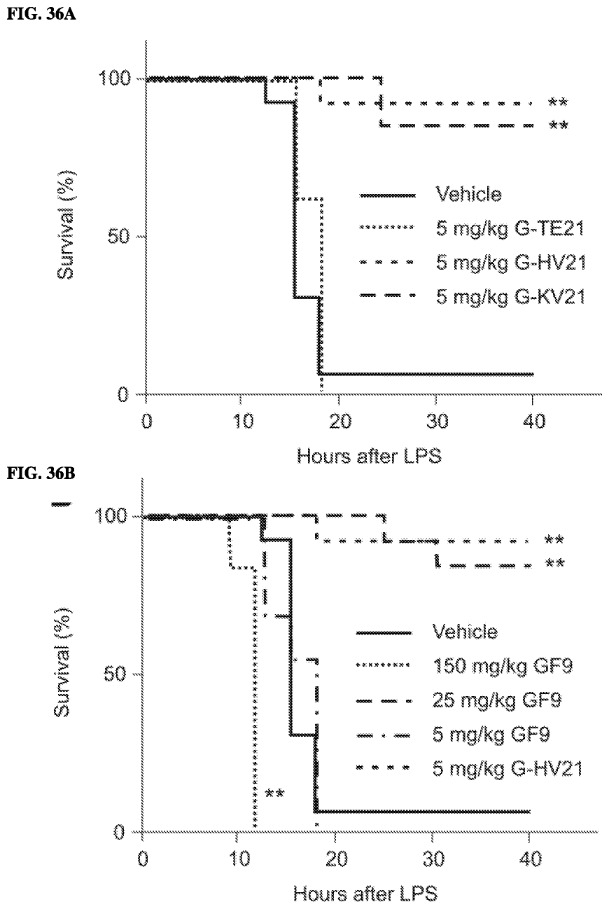

This example demonstrates that at this dose, G-HV21 and G-KV21 but not GF9 and G-TE21 inhibit LPS-stimulated cytokine production in vivo. This example further demonstrates that at this dose, G-HV21 and G-KV21 but not GF9 and G-TE21 protect mice from LPS-induced septic shock and prolongs survival of septic mice. See FIGS. 35 and 36A-B.

### Example 10A: Lung Cancer Tumor Xenografts in Nude Mice and In Vivo Tumor Growth Studies

Animal efficacy studies were performed in human xenograft mouse models of NSCLC using female 6-8 week old NU/J mice from the Jackson Laboratory (Bar Harbor, Me.) using the standard, well-known in the art methods as described in Sigalov. Int Immunopharmacol 2014, 21:208-219 and disclosed in Wu, et al. U.S. Pat. No. 8,415,453 and Sigalov U.S. Pat. No. 8,513,185.

Animal efficacy studies were performed using female 6-8 week old NU/J mice from the Jackson Laboratory (Bar Harbor, Me.). Animals were handled as specified in the USDA Animal Welfare Act (9 CFR, Parts 1, 2, and 3) and as described in the Guide for the Care and Use of Laboratory Animals from the National Research Council. Human lung carcinoma cell lines H292 and A549 were obtained from ATCC. Tumor cells in culture were harvested and resuspended in a 1:1 ratio of RPMI 1640 and Matrigel (BD Biosciences, San Jose, Calif.). NSCLC xenografts were established by injecting subcutaneously into the right flanks 5×10⁶ viable cells per mouse. Tumor volumes were calculated with caliper measurements using the formula V=π/6 (length×width×width). When tumor volumes reached an average of 200 mm³, tumor-bearing animals were randomized into groups of 10, and dosing of PBS (vehicle), paclitaxel (PTX) or peptides G-TE21, G-HV21 and G-KV21 was initiated. All tested formulations were intraperitoneally (i.p.) injected at indicated doses and administration schedule. Clinical observations, body weights and tumor volume measurements were made 3 times weekly. Tumor volumes were analyzed using repeated measures ANOVA followed by Bonferroni test. Data points were represented as mean tumor volume±SEM. Antitumor effects were expressed as the percentage of T/C (treated versus control), dividing the tumor volumes from treatment groups with the control groups and multiplied by 100. According to the National Cancer Institute (NCI) standards (see e.g., Johnson, et al. Br J Cancer 2001, 84:1424-1431), a % T/C≤42 is indicative of antitumor activity. At the end of the experiment, the animals were sacrificed and the tumors were excised and weighed.

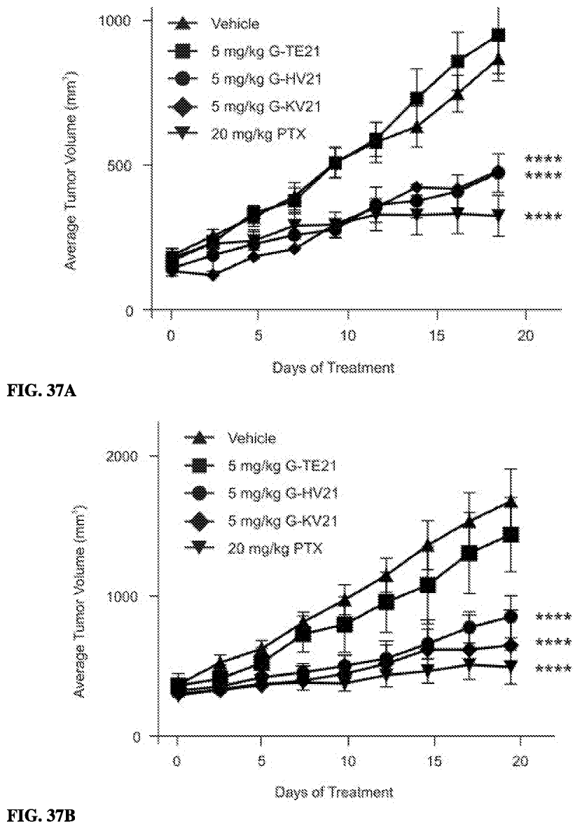

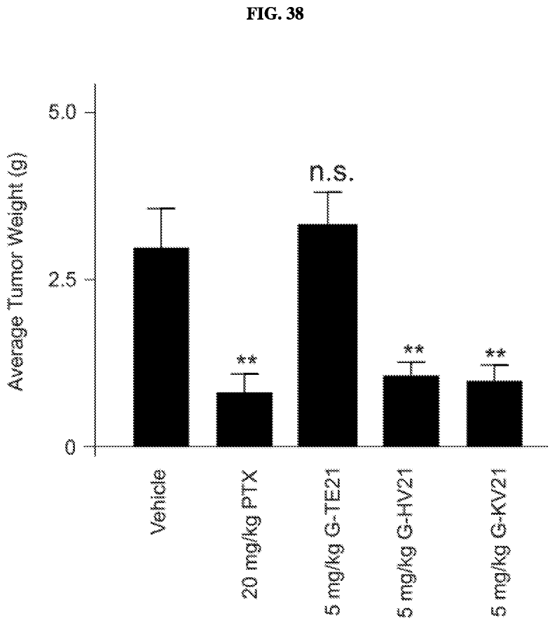

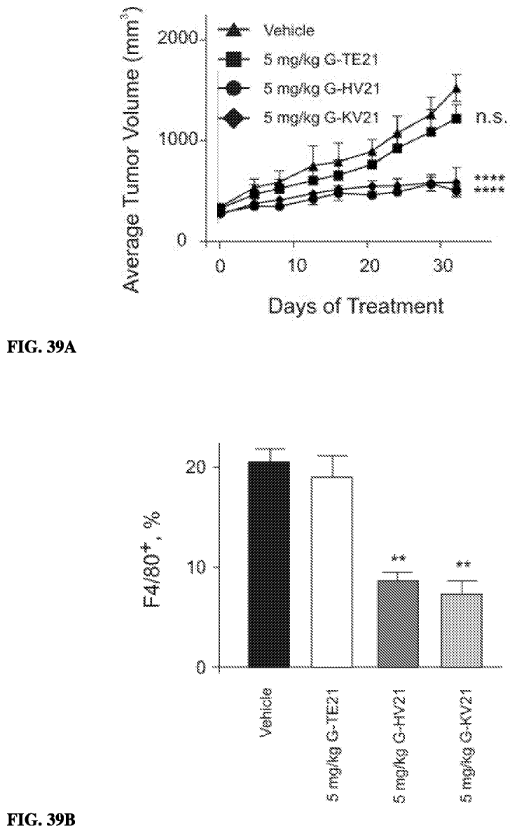

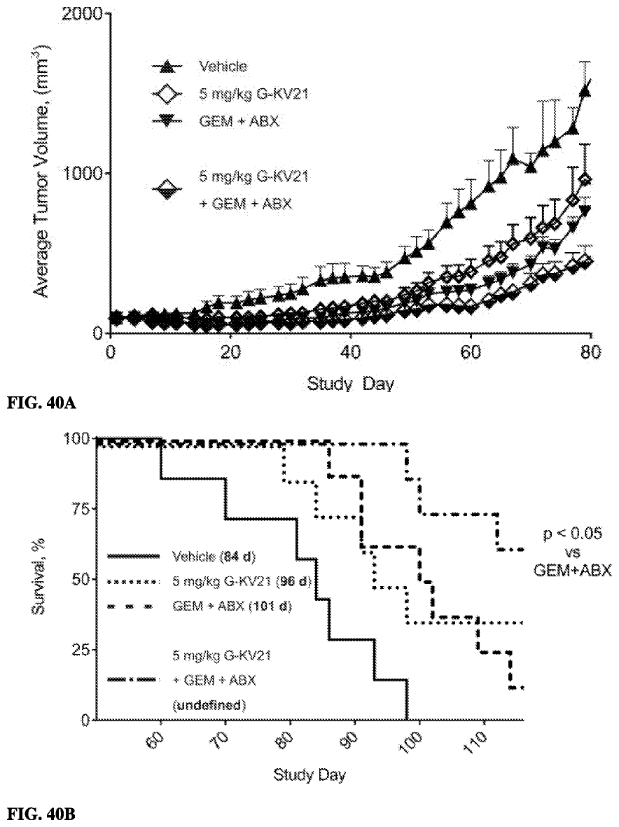

This example demonstrates that G-HV21 and G-KV21 but not G-TE21 inhibit tumor growth in two human NSCLC xenograft mouse models. See FIGS. 37 and 38.

### Example 11A: Pancreatic Cancer Tumor Xenografts in Nude Mice and In Vivo Tumor Growth and Survival Studies

In order to demonstrate that the TREM-1-related TRIOPEP peptides are effective in inhibiting TREM-1-mediated cell activation and reducing pancreatic tumor (PC) growth, animal efficacy studies were performed in human xenograft mouse models of PC using 5-6 week old female athymic nude-Foxn1^{nu} mice obtained from Envigo (formerly Harlan, Inc.) using the standard, well known in the art methods as described in Shen and Sigalov. Mol Pharm 2017, 14:4572-4582.

### Animal Studies

Mice were implanted subcutaneously into the right flank with 5×10⁶ AsPC-1, BxPC-3, CAPAN-1 or PANC-1 cells in equal parts of serum-free growth medium and Matrigel. Mice were monitored daily and tumor measurements were taken along the length and width using Vernier calipers twice weekly until sacrifice. Tumor volumes were calculated using a modified ellipsoidal formula: (Length×Width²)/2. In one embodiment, when the AsPC-1, BxPC-3 and CAPAN-1 tumors reached a calculated volume of approximately 150-200 mm³, mice were sorted into treatment groups and PBS (vehicle), PTX, G-HV21, G-KV21 or G-TE21 were i.p. injected once daily for 5 days per week at indicated doses. Treatment persisted for 31 days for AsPC-1-containing mice and 29 days for mice containing established BxPC-3 and Capan-1 xenograft tumors. In one embodiment, when the PANC-1 tumors reached a calculated volume of approximately 150-200 mm³, mice were sorted into treatment groups and i.p. dosing with PBS (vehicle), chemotherapy (100 mg/kg gemcitabine+10 mg/kg Abraxane, “GEM+ABX”) either with (G-KV21+GEM+ABX) or without (GEM+ABX) 5 mg/kg G-KV21 was started. Treatment with GEM+ABX applied once daily at days 1, 4, 8, 11 and 15. Treatment with PBS or G-KV21 persisted for 30 days once daily for 5 days per week. Mice were humanely sacrificed when individual tumors exceeded 1500 (BxPC-3) or 2000 (PANC-1) mm³.

### Immunohistochemistry

Mice containing BxPC-3 tumors were humanely euthanized for necropsy at the end of the study. Excised tumors were fixed using 10% neutral buffered formalin for 1-2 days, processed for paraffin embedding, and sectioned at 4 m. Antigen retrieval for F4/80 was achieved using Proteinase K (Dako North America). Sections were blocked for peroxidase and alkaline phosphatase activity using Dual Endogenous Enzyme Block (Dako North America). Sections were then incubated with Protein Block (Dako North America) followed by primary antibody F4/80 (1:2000, AbD Serotec) diluted using 1% bovine serum albumin in Tris-buffered saline. Afterward, sections were incubated using EnVision+ secondary antibodies (Dako North America), followed by 3,3′-diaminobenzidine in chromogen solution (Dako North America) and counterstained using hematoxylin (Dako North America). Quantitative analysis of intratumoral F4/80 staining was determined using Visiopharm software.

### Cytokine Detection

Blood was collected on study days 1 and 8 and processed into serum. Serum cytokines were analyzed by Quantibody Mouse Cytokine Array Q1 kits (RayBiotech) according to the manufacturer's instructions.

### Statistical Analysis

Results are expressed as the mean±SEM. Statistical differences were analyzed using analysis of variance with Bonferroni adjustment unless otherwise noted. The Kaplan-Meier method was used to estimate survival as a function of time, and survival differences were analyzed by the log-rank test. p values less than 0.05 were considered significant.

This example demonstrates that G-HV21 and G-KV21 but not G-TE21 inhibit tumor growth in three human PC xenograft mouse models. This example further demonstrates that TREM-1 blockade using G-HV21 and G-KV21 reduces the macrophage infiltration into the tumor. This example further demonstrates that treatment with G-KV21 does not affect the macrophage infiltration into the tumor. This example further demonstrates that being applied with chemotherapy, TREM-1/TRIOPEP sensitizes the PANC-1 tumor to chemotherapy and significantly prolongs survival. See FIGS. 36A-B (shown for BxPC-3) and 40A.

### Example 12A: Mouse Tolerability Studies

Mouse tolerability studies were performed in healthy C57BL/6 mice using the standard, well-known in the art methods as described in Sigalov. Int Immunopharmacol 2014, 21:208-219.

Naïve, female C57BL/6 mice at 8 to 10 weeks of age (18 to 21 g) from the Jackson Laboratory were used. Animals were randomly grouped (5 mice per group) and i.p. injected with 400 mg/kg G-HV21, G-KV21 or G-TE21. Clinical observations and body weights were made twice daily.

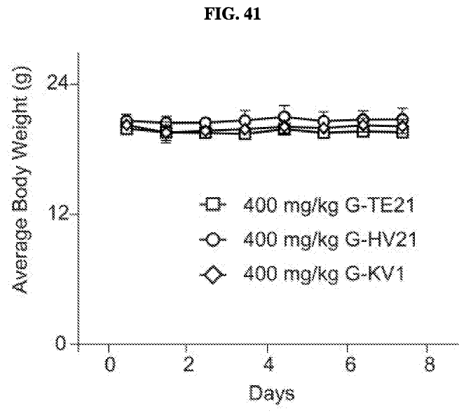

This example demonstrates that G-HV21, G-KV21 and G-TE21 all are non-toxic and well tolerated in healthy mice at doses of up to at least 400 mg/kg. See FIG. 41.

### Example 13A: Haemodynamic Studies in Septic Rats

The role of TREM-1-related trifunctional peptides in further models of septic shock, is investigated by performing LPS- and cecal ligation and puncture (CLP)-induced endotoxinemia experiments in rats. The experiments can be conducted analogously to those described in Gibot, et al. Infect Immun 2006, 74:2823-2830 and disclosed in Faure, et al. U.S. Pat. No. 8,013,116; Faure, et al. U.S. Pat. No. 9,273,111; and Sigalov U.S. Pat. No. 8,513,185.

### LPS-Induced Endotoxinemia

Animals are randomly grouped (n=10-20) and treated with Escherichia coli LPS (0111:B4, Sigma-Aldrich, Lyon, France) i.p. in combination with G-HV21, G-KV21 or G-TE21 at various concentrations.

### CLP Polymicrobial Sepsis Model

Rats (n=6-10 per group) are anesthetized by i.p. administration of ketamine (150 mg/kg). The caecum is exposed through a 3.0-cm abdominal midline incision and subjected to a ligation of the distal half followed by two punctures with a G21 needle. A small amount of stool is expelled from the punctures to ensure potency. The caecum is replaced into the peritoneal cavity and the abdominal incision closed in two layers. After surgery, all rats are injected s.c. with 50 mL/kg of normal saline solution for fluid resuscitation. G-HV21, G-KV21 or G-TE21 are then administered at various concentrations.

### Haemodynamic Measurements in Rats

Immediately after LPS administration as well as 16 hours after CLP, arterial BP (systolic, diastolic, and mean), heart rate, abdominal aortic blood flow, and mesenteric blood flow are recorded. Briefly, the left carotid artery and the left jugular vein are cannulated with PE-50 tubing. Arterial BP is continuously monitored by a pressure transducer and an amplifier-recorder system (IOX EMKA Technologies, Paris, France). Perivascular probes (Transonic Systems, Ithaca, N.Y.) are wrapped up the upper abdominal aorta and mesenteric artery, allowed to monitor their respective flows by means of a flowmeter (Transonic Systems). After the last measurement (4^{th} hour after LPS and 24^{th} hour after CLP), animals are sacrificed by an overdose of sodium thiopental i.v. (intravenously).

### Biological Measurements

Blood is sequentially withdrawn from the left carotid artery. Arterial lactate concentrations and blood gases analyses are performed on an automatic blood gas analyser (ABL 735, Radiometer, Copenhagen, Denmark). Concentrations of TNF-alpha and IL-1beta in the plasma are determined by an ELISA test (Biosource, Nivelles, Belgium) according to the recommendations of the manufacturer. Plasmatic concentrations of nitrates/nitrites are measured using the Griess reaction (R&D Systems, Abingdon, UK).

### Statistical Analyses

Between-group comparisons are performed using Student's t tests. All statistical analyses are completed with Statview software (Abacus Concepts, Calif.).

### Example 14A: Attenuation of Intestinal Inflammation in Animal Models of Colitis

In order to demonstrate that the TREM-1-related TRIOPEP formulations are effective in inhibiting TREM-1-mediated cell activation in animal models of colitis, the experiments can be conducted analogously to those described in Schenk, et al. J Clin Invest 2007, 117:3097-3106 and disclosed in Faure, et al. U.S. Pat. No. 8,013,116; Faure, et al. U.S. Pat. No. 9,273,111 and Sigalov U.S. Pat. No. 8,513,185.

### Mice

C57BL/6 mice, purchased from Harlan, and C57BL/6 RAG2−/− mice, bred in a specific pathogen-free (SPF) animal facility, are used at 8-12 weeks of age. All experimental mice are kept in micro-isolator cages in laminar flows under SPF conditions.

### Mouse Models of Colitis

For experiments involving the adoptive T cell transfer model, colitis is induced in C57BL/6 RAG2−/− mice by adoptive transfer of sorted CD4+CD45RBhigh T cells. Briefly, CD4+ T cells are isolated from splenocytes from C57BL/6 mice, and after osmotic lysis of erythrocytes, CD4+ T cells are enriched by a negative MACS procedure for CD8alpha and B220 (purified, biotinylated, hybridoma supernatant) using avidin-labeled magnetic beads (Miltenyi Biotec). Subsequently, the CD4+ T cell-enriched fraction is stained and FACS sorted for CD4+(RM4-5; BD Biosciences—Pharmingen), CD45RBhi (16A; BD Biosciences—Pharmingen), and CD25− (PC61; eBioscience) naive T cells. Each C57BL/6 RAG2−/− mouse is injected i.p. with 1×105 syngeneic CD4+CD45RBhighCD25− T cells. Colitic mice are sacrificed and analyzed on day 14 after adoptive transfer.

For experiments involving the dextran sodium sulfate (DSS) colitis model, C57BL/6 mice are given autoclaved tap water containing 3% DSS (DSS salt, reagent grade, mol wt: 36-50 kDa; MP Biomedicals) ad libitum over a 5-day period. The consumption of 3% DSS is measured. DSS is replaced thereafter by normal drinking water for another 4 days. Mice are euthanized and analyzed at the end of the 9-day experimental period.

### Treatment

Upon colitis induction, either starting on day 0 or after onset of colitis on day 3, mice are treated with G-HV21, G-KV21 or G-TE21 i.p. injected at various concentrations in 200 ul saline.

### Colitis Scoring

At the end of the experiments, the colon length is measured from the end of the cecum to the anus. Fecal samples are tested for occult blood using hemo FEC (Roche) tests (score 0, negative test; 1, positive test and no rectal bleeding; 2, positive test together with visible rectal bleeding). The colon is divided into 2 parts. From each mouse, identical segments from the distal and proximal colon are taken for protein and RNA isolation and histology, and frozen tissue blocks are prepared for subsequent analysis. Histological scoring of paraffin-embedded H&E-stained colonic sections is performed in a blinded fashion independently by 2 pathologists. To assess the histopathological alterations in the distal colon, a scoring system is established using the following parameters: (a) mucin depletion/loss of goblet cells (score from 0 to 3); (b) crypt abscesses (score from 0 to 3); (c) epithelial erosion (score from 0 to 1); (d) hyperemia (score from 0 to 2); (e) cellular infiltration (score from 0 to 3); and (f) thickness of colonic mucosa (score from 1 to 3). These individual histology scores are added to obtain the final histopathology score for each colon (0, no alterations; 15, most severe signs of colitis).

### RNA Isolation and RT-PCR

RNA is isolated from intestinal tissue samples preserved in RNAlater (QIAGEN), using the RNAeasy Mini Kit (QIAGEN). RT-PCR is performed with 400 ng RNA each, using the TaqMan Gold RT-PCR Kit (Applied Biosystems). Primers are designed as follows: mouse TREM-1, forward 5′-GAGCTTGAAGGATGAGGAAGGC-3′ and reverse 5′-CAGAGTCTGTCACTTGAAGGTCAGTC-3′; mouse TNF, forward 5′-GTAGCCCACGTCGTAGCAAA-3′ and reverse 5′-ACGGCAGAGAGGAGGTTGAC-3′; mouse beta-actin, forward 5′-TGGAATCCTGTGGCATCCATGAAAC-3′ and reverse 5′-TAAAACGCAGCTCAGTAACAGTCCG-3′; human TREM-1, forward 5′-CTTGGTGGTGACCAAGGGTTTTTC-3′ and reverse 5′-ACACCGGAACCCTGATGATATCTGTC-3′; human TNF, forward 5′-GCCCATGTTGTAGCAAACCC-3′ and reverse 5′-TAGTCGGGCCGATTGATCTC-3′; human GAPDH, forward 5′-TTCACCACCATGGAGAAGGC-3′ and reverse 5′-GGCATGGACTGTGGTCATGA-3′. PCR products are semiquantitatively analyzed on agarose gels.

Human TREM-1 and mouse TREM-1 and TNF expression is also assessed by real-time PCR using the TREM-1 QuantiTect primer assay system and QuantiTect SYBR green PCR Kit (both from QIAGEN). GAPDH is used to normalize TREM-1 and TNF expression levels. DNA is amplified on a 7500 Real-Time PCR system (Applied Biosystems), and the increase in gene expression is calculated using Sequence Detection System software (Applied Biosystems).

### Western Blot Analysis

Protein samples are separated on a denaturing 12% acrylamide gel, followed by transfer to nitrocellulose filter and probing with the primary antibody. Anti-TREM-1 (polyclonal goat IgG, 0.1 ug/ml; R&D Systems) or anti-tubulin (clone B-5-1-2, 1:5,000; Sigma-Aldrich) is used as primary reagent. As secondary antibodies, HRP-labeled donkey anti-goat Ig (1:2,000; The Binding Site) and goat anti-mouse Ig (1:4,000; Sigma-Aldrich) are used. Binding is detected by chemiluminescence using a Super Signal West Pico Kit (Pierce).

### Statistics

The unpaired 2-tailed Student t test is used to compare groups; P values less than 0.05 are considered significant.

### Example 15A: Autophage Activity and Colitis in Mice

In order to further demonstrate that the TREM-1-related TRIOPEP formulations are effective in inhibiting TREM-1-mediated cell activation in animal models of colitis, the experiments can be conducted analogously to those described in Kokten, et al. J Crohns Colitis 2018, 12:230-244 and disclosed in Faure, et al. U.S. Pat. No. 8,013,116; and Faure, et al. U.S. Pat. No. 9,273,111.

### Animals

In vivo experiments are performed as recommended by the US National Committee on Ethics Reflection Experiment [described in the Guide for Care and Use of Laboratory Animals, NIH, MD, 1985]. The experiments are performed on 25 adult male C57BL/6 mice [Janvier Labs, Le Genest-Saint-Isle, France] and 10 adult male Trem-1 knock-out [TREM-1 KO] C57BL/6 mice [INSERM U1116, Inotrem Laboratory, Nancy, France], all aged between 7 and 9 weeks. The animals are housed at 22-23° C., with a 12 h/12 h light/dark cycle, and ad libitum access to food and water.

### Induction of Colitis, Treatment and Assessment of Disease Activity Index

Colitis is induced by administration of 3% dextran sulfate sodium [DSS, molecular weight 36,000-50,000, MP Biomedical, Strasbourg, France] dissolved in water for 5 days. DSS is replaced thereafter by normal drinking water for another 5 days. Either G-HV21, G-KV21, G-TE21 or the vehicle alone, used as control, are i.p. administered 2 days before colitis induction and then once daily until the last day of DSS administration, at different concentrations in 200 L of saline. This dose is chosen after having performed dose-response experiments. Bodyweight, physical condition, stool consistency, water/food consumption and the presence of gross and occult blood in excreta and at the anus are determined daily. The DAI is also calculated daily by scoring bodyweight loss, stool consistency and blood in the stool on a 0 to 4 scale. 41 The overall index corresponds to the weight loss, stool consistency and rectal bleeding scores divided by three, and thus ranges from 0 to 4.

### Collection of Colon Tissue and Fecal Samples

Ten days after the initiation of colitis with DSS, the mice are sacrificed by decapitation. The colon is quickly removed, opened along its length and gently washed in PBS [2.7 mmol/L KCl, 140 mmol/L NaCl, 6.8 mmol/L Na2HPO4.2H2O, 1.5 mmol/L KH2PO4, pH 7.4]. For histological assessment samples are fixed overnight at 4° C. in 4% paraformaldehyde solution and embedded in paraffin. For protein extractions samples are frozen in liquid nitrogen [−196° C.] and stored at −80° C. For the gut microbiota analysis, whole fecal pellets are collected daily in sterile tubes and immediately frozen at −80° C. until analysis.

### Histological Assessment and Scoring

Colitis is histologically assessed on 5 m sections stained with hematoxylin-eosin-saffron [HES] stain. The histological colitis score is calculated blindly by an expert pathologist.

### Endoscopic Assessment and Scoring

Endoscopy is performed on the last day of the study, just before the mice are sacrificed. Prior to the endoscopic procedure, mice are anaesthetized by isoflurane inhalation. The distal colon [3 cm] and the rectum are examined using a rigid Storz Hopkins II miniendoscope [length: 30 cm; diameter: 2 mm; Storz, Tuttlingen, Germany] coupled to a basic Coloview system [with a xenon 175 light source and an Endovision SLB Telecam; Storz]. Air is insufflated via a 9-French gauge over-tube and a custom, low-pressure pump with manual flow regulation [Rena Air 200; Rena, Meythet, France]. All images are displayed on a computer monitor and recorded with video capture software [Studio Movie Board Plus from Pinnacle, Menlo Park, Calif.]. The endoscopy score is calculated from three subscores: the vascular pattern [scored from 1 to 3], bleeding [scored from 1 to 4] and erosions/ulcers [scored from 1 to 4].

### Western Blot Analysis

Total protein is extracted from the frozen colon samples by lysing homogenized tissue in a radioimmunoprecipitation assay [RIPA] buffer [0.5% sodium deoxycholate, 0.1% sodium dodecyl sulfate [SDS] and 1% NP-40] supplemented with protease inhibitors [Roche Diagnostics, Mannheim, Germany]. Protein is then quantified using the bicinchoninic acid assay method. For each mouse, a total of 20 μg of protein is transferred to a 0.45 m polyvinylidene fluoride [PVDF] or 0.45 m nitrocellulose membrane following electrophoretic separation on a denaturing acrylamide gel. The membrane is blocked with 5% w/v non-fat powdered milk or 5% w/v bovine serum albumin [BSA] diluted in Tris-buffered saline with 0.1% v/v Tween® 20 [TBST] for 1 h at room temperature. The PVDF or nitrocellulose membranes are then incubated overnight at 4° C. with various primary antibodies diluted in either 5% w/v nonfat powdered milk or 5% w/v BSA, TBST. After washing in TBST, the appropriate HRP-conjugated secondary antibody is added and the membrane is incubated for 1 h at room temperature. After further washing in TBST, the proteins are detected using an ECL or ECL PLUS kit [Amersham, Velizy-Villacoublay, France]. Glyceraldehyde 3-phosphate dehydrogenase [GAPDH] is used as an internal reference control.

Enzyme-Linked Immunosorbent Assay [ELISA] for Analysis of Soluble TREM-1 [sTREM-1]

At the time of animal sacrifice, whole blood from each mouse is collected into heparinized tubes. These tubes are centrifuged at 3,000 g for 10 min at 4° C. to collect the supernatants, which are stored at −80° C. until use. Plasma concentration of sTREM-1 is determined by a sandwich ELISA technique using the Quantikine kit assay [RnD Systems, Minneapolis, Minn., USA] according to the manufacturers' instructions. Briefly, samples are incubated with a monoclonal antibody specific for TREM-1 pre-coated onto the wells of a microplate. Following a wash, to eliminate the unbound substances, an enzyme-linked polyclonal antibody specific for TREM-1 is added to the wells. After washing away the unbound conjugate, a substrate solution is added to the wells. Color development is stopped and optical density of each well is determined within 30 min using a microplate reader [Sunrise, Tecan, Mannedorf, Switzerland] set to 450 nm, with a wavelength correction set to 540 nm. All measurements are performed in duplicate and the sTREM-1 concentration is expressed in pg/ml.

### Reverse Transcription-Quantitative Polymerase Chain Reaction

Total RNA is purified from the frozen colon samples with the RNeasy Lipid Tissue kit following the recommendation of Qiagen [Courtaboeuf, France], which includes treatment with DNase. To check for possible DNA contamination of the RNA samples, reactions are also performed in the absence of Omniscript RT enzyme [Qiagen]. Reverse transcription is performed using PrimeScript™ RT Master Mix [TAKARA Bio, USA] according to the manufacturer's recommendations with 200 ng of RNA in a 10 μL reaction volume. PCR is then carried out from 2 μL of cDNA with SYBR® Premix Ex Taq™ [Tli RNaseH Plus] [TAKARA Bio, USA] according to the manufacturer's recommendations in a 20 μL reaction volume, with reverse and forward primers at a concentration of 0.2 μM. Specific amplifications are performed using the following primers: TREM-1, forward 5′-CTGTGCGTGTTCTTTGTC-3′ and reverse 5′-CTTCCCGTCTGGTAGTCT-3′. Quantification is performed with RNA polymerase II [Pol II] as an internal standard with the following primers: forward 5′-AGCAAGCGGTTCCAGAGAAG-3′ and reverse 5′-TCCCGAACACTGACATATCTCA-3′. Temperature cycling for TREM-1 is 30 s at 95° C. followed by 40 cycles consisting of 95° C. for 5 s and 59° C. for 30 s. Temperature cycling for RNA polymerase II is 30 s at 95° C. followed by 40 cycles consisting of 95° C. for 5 s and 60° C. for 30 s. Results are expressed as arbitrary units by calculating the ratio of crossing points of amplification curves of TREM-1 and internal standard by using the δδCt method.

### Microbiota Analysis

For the pharmacologically [with TREM-1/TRIOPEP treatment] inhibition of TREM-1, total DNA is extracted from three pooled fecal pellets from each group of mice [day 0 to day 10; n=33 samples]. For microbiota analysis by MiSeq sequencing, the V3-V4 region [519F-785R] of the 16S rRNA gene is amplified with the primer pair S-DBact-0341-b-S-17/S-D-Bact-0785-a-A-21.45 The following quality filters are applied: minimum length=300 base pairs [bp], maximum length=600 bp and minimum quality threshold=20. This filtering yields an average [range] of 25600 reads/samples [14,553-35,490] for further analysis. High-quality reads are pooled, checked for chimeras [using uchime46], and grouped into operational taxonomic units [OTUs][based on a 97% similarity threshold] using USEARCH 8.0.47 Singletons and OTUs representing less than 0.02% of the total number of reads are removed, and the phylogenetic affiliation of each OTU is assessed with Ribosomal Database Project's taxonomy48 from the phylum level to the species level. The mean [range] number of detected OTUs per sample is 324 [170-404]. In the experiments involving Trem-1 KO mice, similar methods are applied but total DNA is extracted from individual fecal pellets of each mouse from the four groups of animals at baseline [before DSS treatment] and at day 10 [after DSS treatment] [n=37 samples]. Following MiSeq sequencing of the V3-V4 region of the 16S rRNA gene, yielding 2,143,457 raw reads, quality filtering is applied [minimum length=200 bp, maximum length=600 bp and minimum quality threshold=20] and an average [range] of 11,560 reads/samples [7,560-18,495] is kept for further analysis. The mean [range] number of detected OTUs per sample is 599 [131-798].

### Statistical Analysis

A two-tailed Student t test is used to compare two groups and a one-way analysis of variance [ANOVA] is used to compare three or more groups. Bonferroni or Tamhane post hoc tests are applied, depending on the homogeneity of the variance. The threshold for statistical significance is set to p<0.05. The statistical language R is used for data visualization and to perform abundance-based principal component analysis [PCA] and interclass PCA associated with Monte-Carlo rank testing on the bacterial genera.

### Example 16A: Modulation of the TREM-1 Pathway During Severe Hemorrhagic Shock in Rats

In order to demonstrate that the TREM-1-related TRIOPEP formulations are effective in inhibiting TREM-1-mediated cell activation and preventing organ dysfunction and improving survival in rats during severe hemorrhagic shock, the experiments can be conducted analogously to those described in Gibot, et al. Shock 2009, 32:633-637 and disclosed in Faure, et al. U.S. Pat. No. 8,013,116; Faure, et al. U.S. Pat. No. 9,273,111, and Sigalov. U.S. Pat. No. 8,513,185.

### Animals

Adult male Wistar rats (250-300 g) are purchased from Charles River Laboratories (Wilmington, Mass., USA). After 1 week of acclimatization, rats are fasted 12 h before the experiments and are allowed free access to water. All the studies described in the succeeding sentences comply with the regulations concerning animal use and care published by the National Institutes of Health.

### Hemorrhagic Shock Model

Hemorrhagic shock is induced by bleeding from a heparinized (10 UI/mL) carotid artery catheter. Briefly, the rats are anesthetized (50 mg/kg pentobarbital sodium, i.p.) and kept on a temperature-controlled surgical board (37° C.). A tracheostomy is performed, and the animals are ventilated supine (tidal volume, 7-8 mL/kg; rodent ventilator no. 683; Harvard Apparatus, Holliston, Mass.) with a fraction of inspired oxygen of 0.3 and a respiratory rate of 60 breaths per minute. Anesthesia and respiratory support are maintained during the whole experiment. The left carotid artery and the left jugular vein are cannulated with PE-50 tubing. Arterial blood pressure is continuously monitored by a pressure transducer and an amplifier-recorder system (IOX EMKA Technologies, Paris, France). After a 30-min stabilization period, blood is drawn in 10 to 15 min via the carotid artery catheter until MAP reached 40 mmHg. Blood is kept at 37° C., and MAP is maintained between 35 and 40 mm Hg during 60 min. Rats are then allocated randomly (n=10-12 per group) to receive 0.1 mL of either saline (isotonic sodium chloride solution), G-HV21, G-KV21 or G-TE21 at various concentrations in 0.1 mL of saline solution over 1 min via the jugular vein (H0). Shed blood and ringer lactate (volume=3× shed volume) are then infused via the jugular vein in 60 min, and rats are observed for a 4-h period before being killed by pentobarbital sodium overdose. Killing occurs earlier if MAP decreased to less than 35 mm Hg.

### Arterial Blood Gas, Lactate, and Cytokines

Arterial blood gas and lactate concentrations are determined hourly on an automatic blood gas analyzer (ABL 735; Radiometer, Copenhagen, Denmark). Concentrations of TNF-alpha and IL-6 and sTREM-1 in the plasma are determined in triplicate by enzyme-linked immunosorbent assay (Biosources, Nivelles, Belgium; RnD Systems, Lille, France).

### Bacterial Translocation

Rats are killed under anesthesia, and mesenteric lymph node (MLN) complex, spleen, and blood are aseptically removed 4 h after the beginning of reperfusion (or earlier if MAP decreased <35 mm Hg). Homogenates of MLN and spleen and serial blood dilutions are plated and incubated overnight at 37° C. on Columbia blood agar plates (in carbon dioxide and anaerobically) and Macconkey agar (in air). Visible colonies are then counted.

### Pulmonary Integrity

Additional groups of rats (n=4) are subjected to the same procedure but are also infused via the tail vein with fluorescein isothiocyanate (FITC)-albumin (5 mg/kg in 0.3 mL of phosphate-buffered saline) 2 h after the beginning of reperfusion. Rats in these groups are killed 2 h later with an overdose of sodium pentobarbital (200 mg/kg). Immediately thereafter, the lungs are lavaged three times with 1 mL of phosphate-buffered saline, and blood is collected by cardiac puncture. The bronchoalveolar lavage fluid (BALF) is pooled, and plasma is collected. Fluorescein isothiocyanate-albumin concentrations in BALF and plasma are determined fluorometrically (excitation, 494 nm; emission, 520 nm). The BALF-plasma fluorescence ratio is calculated and used as a measure of damage to pulmonary alveolar endothelial/epithelial integrity as previously described (Yang et al. Crit Care Med 2004; 32:1453-9).

### Statistical Analysis

Data are analyzed using ANOVA or ANOVA for repeated measures when appropriate, followed by Newman-Keuls post hoc test. Survival curves are compared using the log-rank test. A two-tailed value of P less than 0.05 is deemed significant. All analyses are performed with GraphPad Prism software (GraphPad, San Diego, Calif.).

### EXPERIMENTAL SECTION

Cell lines and reagents. Human pancreatic cancer cell lines (AsPC-1, BxPC-3, and Capan-1) were purchased from the ATCC. Sodium cholate, cholesteryl oleate and other chemicals were purchased from Sigma Aldrich Company. 1,2-dimyristoyl-sn-glycero-3-phosphocholine (DMPC), 1,2-dimyristoyl-sn-glycero-3-phospho-(1′-rac-glycerol) (DMPG), 1-palmitoyl-2-oleoyl-sn-glycero-3-phosphocholine (POPC), 1-palmitoyl-2-oleoyl-sn-glycero-3-phospho-(1′-rac-glycerol) (POPG), 1,2-dimyristoyl-sn-glycero-3-phosphoethanolamine-N-(lissamine rhodamine B sulfonyl) (Rho B-PE) and cholesterol were purchased from Avanti Polar Lipids.

Peptide synthesis. The following synthetic peptides were ordered from Bachem Americas, Inc.: one 9-mer peptide GFLSKSLVF (human TREM-1₂₁₃₋₂₂₁, GF9), two 22-mer methionine sulfoxidized peptides PYLDDFQKKWQEEM(O)ELYRQKVE (H4) and PLGEEM(O)RDRARAHVDALRTHLA (H6) that correspond to human apo A-I helices 4 (apo A-I₁₂₃₋₁₄₄) and 6 (apo A-I₁₆₇₋₁₈₈), respectively, and two 31-mer methionine sulfoxidized peptides, GFLSKSLVFPYLDDFQKKWQEEM(O)ELYRQKVE (GE31) and GFLSKSLVFPLGEEM(O)RDRARAHVDALRTHLA (GA31).

Lipopeptide complexes. HDL-mimicking lipopeptide complexes of discoidal (dHDL) and spherical (sHDL) morphology loaded with GF9 (GF9-dHDL and GF9-sHDL, respectively) or an equimolar mixture of GA31 and GE31 (GA/E31-dHDL and GA/E31-sHDL) were synthesized using the sodium cholate dialysis procedure, purified and characterized essentially as previously described. (Sigalov 2014, Shen and Sigalov 2016, Shen and Sigalov 2017) Briefly, in discoidal complexes, the molar ratio was 65:25:3:1:190 corresponding to POPC:POPG:GF9:apo A-I:sodium cholate for GF9-dHDL that contain GF9 and an equimolar mixture of oxidized apo A-I peptides H4 and H6 peptides or 65:25:1:190 corresponding to DMPC:DMPG:GA/E31:sodium cholate for GA/E31-dHDL that contain an equimolar mixture of oxidized peptides GA31 and GE31. In spherical complexes, the molar ratio was 125:6:2:3:1:210 corresponding to POPC:cholesterol:cholesteryl oleate:GF9:apo A-I:sodium cholate for GF9-sHDL that contain GF9 and an equimolar mixture of oxidized apo A-I peptides H4 and H6 or 125:6:2:1:210 corresponding to POPC:cholesterol:cholesteryl oleate:GA/E31:sodium cholate for GA/E31-sHDL that contain an equimolar mixture of oxidized peptides GA31 and GE31.

Mouse xenograft tumor models. All animal studies were performed by Bolder BioPATH and conducted under an approved IACUC protocol. Briefly, 5-6 week old female athymic nude-Foxn1nu mice were obtained from Envigo (formerly Harlan, Inc.). Mice were implanted subcutaneously into the right flank with 5×106 AsPC-1, BxPC-3 or Capan-1 cells in equal parts of serum-free growth medium and Matrigel. Mice were monitored daily and tumor measurements were taken along the length and width using Vernier calipers twice weekly until sacrifice. Tumor volumes were calculated using a modified ellipsoidal formula: (Length×Width2)/2. When tumors reached a calculated volume of approximately 150-200 mm³, mice were sorted into treatment groups and injected intraperitoneally (i.p.) once daily for 5 days per week (5qw) with free or HDL-bound TREM-1 inhibitory GF9 sequences: GF9 (2.5 and 25 mg/kg), GF9-dHDL (2.5 mg/kg), GF9-sHDL (2.5 mg/kg), GA/E31-dHDL (dose equivalent to 4 mg of GF9/kg), and GA/E31-sHDL (dose equivalent to 4 mg of GF9/kg) or with PBS. Treatment persisted for 31 days, 29 days and 29 days for mice containing established AsPC-1, BxPC-3 and Capan-1 xenograft tumors, respectively. Mice were humanely sacrificed when individual tumors exceeded 1500 mm3.

Immunohistochemistry. All staining and quantification procedures were performed by HistoTox Labs. Briefly, mice containing AsPC-1, BxPC-3 and Capan-1 tumors were humanely euthanized for necropsy at the end of the study. Excised tumors were fixed using 10% neutral buffered formalin for 1-2 days, processed for paraffin embedding and sectioned at 4 m. Antigen retrieval for F4/80 was achieved using Proteinase K (Dako North America). Sections were blocked for perioxidase and alkaline phosphatase activity using Dual Endogenous Enzyme Block (Dako North America). Sections were then incubated with Protein Block (Dako North America) followed by primary antibody F4/80 (1:2000, AbD Serotec) diluted using 1% bovine serum albumin in Tris-buffered saline. Afterwards, sections were incubated using EnVision+ secondary antibodies (Dako North America), followed by 3,3′-diaminobenzidine in chromogen solution (Dako North America) and counterstained using hematoxylin (Dako North America). Quantitative analysis of intratumoral F4/80 staining was determined using Visiopharm software.

Cytokine detection. Blood was collected on study days 1 and 8 and processed into serum. Serum cytokines were analyzed by Quantibody Mouse Cytokine Array Q1 kits (RayBiotech) according to the manufacturer's instructions.

Statistical analysis. All statistical analyses were performed using GraphPad Prism 6.0 (GraphPad Software). Percent treatment/control (T/C) values were calculated using the following formula: % T/C=100×ΔT/ΔC where T and C are the mean tumor volumes of the drug-treated and control groups, respectively, on the final day of the treatment; ΔT is the mean tumor volume of the drug-treated group on the final day of the treatment minus mean tumor volume of the drug-treated group on initial day of dosing; and ΔC is the mean tumor volume of the control group on the final day of the treatment minus mean tumor volume of the control group on initial day of dosing.

Results are expressed as the mean±SEM. Statistical differences were analyzed using analysis of variance with Bonferroni adjustment unless otherwise noted. The Kaplan-Meier method was used to estimate survival as a function of time, and survival differences were analyzed by the log-rank test. p values less than 0.05 were considered significant.

Sequence accession numbers. Accession numbers (UniProtKB/Swiss-Prot knowledgebase, http://www.uniprot.org/) for the protein sequences discussed in this Research Article is as the follows: human TREM-1, Q9NP99; human apo A-I, P02647.

### EXAMPLES

The following non-limiting Examples are put forth so as to provide those of ordinary skill in the art with illustrative embodiments as to how the compounds, compositions, articles, devices, and/or methods claimed herein are made and evaluated. The Examples are intended to be purely exemplary of the invention and are not intended to limit the scope of what the inventor regard as his invention. Efforts have been made to ensure accuracy with respect to numbers (e.g., amounts, temperature, etc.) but some errors and deviations should be accounted for.

Standard methods of isolation, synthesis, modification, purification, and characterization of synthetic peptides and compounds are well-known in the art (see e.g., Elmore U.S. Pat. No. 4,749,742; Sigalov U.S. Pat. No. 8,513,185; Sigalov U.S. Pat. No. 9,981,004, each of which is herein incorporated by reference in it's entirety).

### I. Examples of Trifuncitonal Peptides as Part of SHDLs

### Example 1: Exemplary Synthesis and Modification of Peptides

This example demonstrates one embodiment of synthesized TREM-1 inhibitory SCHOOL peptide GF9 and TREM-1-related trifunctional peptides (TREM-1/TRIOPEP) GA31 and GE31.

The first step is to synthesize the 9 amino acids-long peptide comprising a portion of a TREM-1 transmembrane domain sequence (the TREM-1₂₁₃₋₂₂₁). Although it is not necessary to understand the mechanism of an invention, it is believed that this peptide affects the TREM-1/DAP-12 receptor complex assembly, inhibits the TREM-1 signaling pathway and functions to treat and/or prevent a TREM-1-related disease or condition. In one embodiment, the amino acid sequence of a peptide comprises GFLSKSLVF (SEQ ID NO. 2), hereafter referred to as GF9. In another embodiment, the amino acid sequence of a peptide comprises GFLSGSLVF wherein, Lys₅ of GF9 is substituted with Gly₅, hereafter referred to as a “GF9-G” or a “control peptide”. Although it is not necessary to understand the mechanism of an invention, it is believed that the positively charged Lys₅ in GF9 a salt bridge to an aspartic acid residue in the transmembrane domain of the DAP-12 chain. Thus, GF9-G may be considered a “control peptide” because of the Lys₅ substitution with Gly₅.

The second step is to synthesize the 22 amino acids-long peptide with sulfoxidized methionine residue that corresponds to human apo A-I 22 amino acids-long helix 4. Although it is not necessary to understand the mechanism of an invention, it is believed that a 22 amino acids-long apo A-I helix 4 peptide sequence with sulfoxidized methionine residue functions to assist in the self-assembly of SLP upon binding to lipid or lipid mixtures and to target the particles to macrophages. In one embodiment, the amino acid sequence of a peptide comprises PYLDDFQKKWQEEM(O)ELYRQKVE where M(O) is a methionine sulfoxide residue, hereafter referred to as PE22. In one embodiment the methionine residue is unmodified.

The third step is to synthesize the 22 amino acids-long peptide with sulfoxidized methionine residue that corresponds to human apo A-I 22 amino acids-long helix 6. Although it is not necessary to understand the mechanism of an invention, it is believed that a 22 amino acids-long apo A-I helix 6 peptide sequence with sulfoxidized methionine residue functions to assist in the self-assembly of SLP upon binding to lipid or lipid mixtures and to target the particles to macrophages and/or SRBI-expressing cells (e.g., hepatocytes, cancer cells). In one embodiment, the amino acid sequence of a peptide comprises PLGEEM(O)RDRARAHVDALRTHLA where M(O) is a methionine sulfoxide residue, hereafter referred to as PA22. In one embodiment the methionine residue is unmodified.

The fourth step is to synthesize the 31 amino acids-long peptide comprising domains A and B where domain A is a 9 amino acids-long TREM-1 inhibitory therapeutic peptide sequence, whereas domain B is a 22 amino acids-long apolipoprotein A-I helix 6 peptide sequence with sulfoxidized methionine residue. Although it is not necessary to understand the mechanism of an invention, it is believed that a 9 amino acids-long TREM-1 inhibitory therapeutic peptide sequence corresponding to a portion of a TREM-1 transmembrane domain sequence affects the TREM-1/DAP-12 receptor complex assembly, inhibits the TREM-1 signaling pathway and functions to treat and/or prevent a TREM-1-related disease or condition, whereas a 22 amino acids-long apolipoprotein A-I helix 6 peptide sequence with sulfoxidized methionine residue functions to assist in the self-assembly of synthetic lipoprotein/lipopeptide particles (SLP) upon binding to lipid or lipid mixtures and to target the particles to TREM-1-expressing macrophages and/or scavenger receptor BI (SR-B1)-expressing cells (e.g., hepatocytes, cancer cells). See FIG. 2.

The fifth step is to synthesize the 31 amino acids-long peptide comprising domains A and B where domain A is a 9 amino acids-long TREM-1 inhibitory therapeutic peptide sequence, whereas domain B is a 22 amino acids-long apolipoprotein A-I helix 4 peptide sequence with sulfoxidized methionine residue. Although it is not necessary to understand the mechanism of an invention, it is believed that a 9 amino acids-long TREM-1 inhibitory therapeutic peptide sequence corresponding to a portion of a TREM-1 transmembrane domain sequence affects the TREM-1/DAP-12 receptor complex assembly, inhibits the TREM-1 signaling pathway and functions to treat and/or prevent a TREM-1-related disease or condition, whereas a 22 amino acids-long apolipoprotein A-I helix 4 peptide sequence with sulfoxidized methionine residue functions to assist in the self-assembly of SLP upon binding to lipid or lipid mixtures and to target the particles to macrophages.

In one embodiment, the 31 amino acids-long peptide comprises domains A and B where domain A is a 9 amino acids-long TREM-1 inhibitory therapeutic peptide sequence, whereas domain B is a 22 amino acids-long apolipoprotein A-I helix 4 peptide sequence with sulfoxidized methionine residue. Although it is not necessary to understand the mechanism of an invention, it is believed that a 9 amino acids-long TREM-1 inhibitory therapeutic peptide sequence corresponding to a portion of a TREM-1 transmembrane domain sequence affects the TREM-1/DAP-12 receptor complex assembly, inhibits the TREM-1 signaling pathway and functions to treat and/or prevent a TREM-1-related disease or condition, whereas a 22 amino acids-long apolipoprotein A-I helix 4 peptide sequence with sulfoxidized methionine residue functions to assist in the self-assembly of synthetic lipoprotein/lipopeptide particles (SLP) upon binding to lipid or lipid mixtures and to target the particles to TREM-1-expressing macrophages. See FIG. 2.

In one embodiment, the amino acid sequence of a trifunctional peptide comprises NH2-Gly-Phe-Leu-Ser-Lys-Ser-Leu-Val-Phe-Pro-Leu-Gly-Glu-Glu-Met-Arg-Asp-Arg-Ala-Arg-Ala-His-Val-Asp-Ala-Leu-Arg-Thr-His-Leu-Ala-OH (i.e., GFLSKSLVFPLGEEMRDRARAHVDALRTHLA, SEQ ID NO:01), hereafter referred to as a TREM-1-related “TRIOPEP” peptide or “TREM-1/TRIOPEP” GA31. See FIG. 2. In another embodiment, the amino acid sequence of a peptide comprises NH2-Gly-Phe-Leu-Ser-Ala-Ser-Leu-Val-Phe-Pro-Leu-Gly-Glu-Glu-Met-Arg-Asp-Arg-Ala-Arg-Ala-His-Val-Asp-Ala-Leu-Arg-Thr-His-Leu-Ala-OH (i.e., GFLSASLVFPLGEEMRDRARAHVDALRTHLA) wherein, Lys₅ of TREM-1/TRIOPEP is substituted with Ala₅, hereafter referred to as a “TREM-1/TRIOPEP GA31-A” or “control peptide GA31-A”.

In one embodiment, the amino acid sequence of a trifunctional peptide comprises NH2-Gly-Phe-Leu-Ser-Lys-Ser-Leu-Val-Phe-Pro-Tyr-Leu-Asp-Asp-Phe-Gln-Lys-Lys-Trp-Gln-Glu-Glu-Met-Glu-Leu-Tyr-Arg-Gln-Lys-Val-Glu-OH (i.e., GFLSKSLVFPYLDDFQKKWQEEMELYRQKVE, SEQ ID NO:02), hereafter referred to as a TREM-1-related “TRIOPEP” peptide or “TREM-1/TRIOPEP” GE31. See FIG. 2. In another embodiment, the amino acid sequence of a peptide comprises NH2-Gly-Phe-Leu-Ser-Ala-Ser-Leu-Val-Phe-Pro-Tyr-Leu-Asp-Asp-Phe-Gln-Lys-Lys-Trp-Gln-Glu-Glu-Met-Glu-Leu-Tyr-Arg-Gln-Lys-Val-Glu-OH (i.e., GFLSASLVFPYLDDFQKKWQEEMELYRQKVE) wherein, Lys₅ of TREM-1/TRIOPEP GE31 is substituted with Ala₅, hereafter referred to as a “TREM-1/TRIOPEP GE31-A” or “control peptide GE31-A”.

Although it is not necessary to understand the mechanism of an invention, it is believed that the positively charged Lys₅ in TREM-1/TRIOPEP forms a salt bridge to an aspartic acid residue in the transmembrane domain of the DAP-12 chain. Thus, TREM-1/TRIOPEP GA-31A may be considered a “control peptide” because of the Lys₅ substitution.

The peptides can be synthesized using a solid phase peptide synthesis (see e.g., Elmore U.S. Pat. No. 4,749,742, herein incorporated by referene in it's entirety). Unprotected unmodified and methionine sulfoxidized peptides can be purchased from specialized companies (i.e., Bachem, Torrance, Calif., USA) with greater than 95% purity as assessed by HPLC. Peptide molecular mass can be checked by matrix-assisted laser desorption ionization mass spectrometry.

To convert methionine residues in unmodified GA31, GE31, GA31-A and GE31-A to methionine sulfoxides, the standard procedure known in the art to prepare protein containing methionine sulfoxides can be also used (see e.g., Elmore U.S. Pat. No. 4,749,742; Sigalov US 20130045161; Sigalov US 20110256224; Sigalov and Stern. FEBS Lett 1998, 433:196-200; Sigalov and Stern. Chem Phys Lipids 2001, 113:133-146, each of which is herein incorporated by referene in it's entirety). Briefly, a purified peptide (about 15 mg) is dissolved in 1 ml of 3 M guanidine-HCl, pH 7.4, and then hydrogen peroxide is added to a final concentration of 300 mM. The mixture is incubated at 20° C. for 15 min, and an oxidized peptide is purified by preparative HPLC using a BioCAD/SPRINT System from PerSeptive Biosystems (Cambridge, Mass., USA), a Vydac C-18 column (22 mm×250 mm) and a two-solvent system: A, trifluoroacetic acid/water (1:1000, v/v); B, trifluoroacetic acid/acetonitrile/water (1:900:100, v/v). The column is heated to 50° C. in a water bath and peptides (modified and unmodified) are eluted at a flow rate of 15 ml/min with 28-49%, 49-53% and 53-73% gradient steps of solvent B over 12, 9 and 12 min, respectively. Then the content of solvent B is increased to 100% over 3 min, and finally decreased to 28% over 2 min. Peaks are identified by analytical HPLC. Analytical HPLC is performed using a Waters Automated Gradient Controller, a Waters 745B Data Processor and a Thermo Separation Products Spectra 100 UV-visible detector, coupled to a Vydac C-18 column (4.6 mm×250 mm) and heated to 50° C. Peptide is eluted with the same two-solvent system at a flow rate of 1.2 ml/min and 28-64% gradient of B over 33 min. Then the content of B is increased to 100% over 2 min, and finally decreased to 28% over 2 min. The HPLC column eluates are monitored by absorbance at 214 nm. Mass spectra of a purified modified peptide is measured using a Voyager Elite STR mass spectrometer from PerSeptive Biosystems (Cambridge, Mass., USA). Conversion of one methionine residue to methionine sulfoxide results in increasing the molecular weight of the peptide by 16 atomic mass units corresponding to an addition of one extra oxygen atom to the peptide molecule.

### Example 2: Preparation and Characterization of Synthetic Lipopeptide Particles

Synthetic lipoprotein/lipopeptide particles (SLP) can be readily reconstituted in vitro from lipids and apolipoproteins. The standard methods of reconstitution and procedures of SLP purification and characterization that are well known in the art and described in Rojas, et al. Biochim Biophys Acta 2018, 1864:2761-2768; Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; and disclosed in Sigalov US 20130045161 and Sigalov US 20110256224, each of which is herein incorporated by referene in it's entirety, were used to reconstitute SLP as spherical or discoidal particles using from peptide and lipid ingredients.

### Reagents

1,2-dimyristoyl-sn-glycero-3-phosphocholine (DMPC), 1,2-dimyristoyl-sn-glycero-3-phospho-(1′-rac-glycerol) (DMPG), 1,2-dimyristoyl-sn-glycero-3-phosphoethanolamine-N-(lissamine rhodamine B sulfonyl) (rhodamine-PE, Rho B-PE), 1-palmitoyl-2-oleoyl-sn-glycero-3-phosphocholine (POPC), 1-palmitoyl-2-oleoyl-sn-glycero-3-phospho-(1′-rac-glycerol) (sodium salt) (POPG), 1,2-dimyristoyl-sn-glycero-3-phosphoethanolamine-N-diethylenetriaminepentaacetic acid (gadolinium salt) (14:0 PE-DTPA (Gd)), and egg yolk L-α-phosphatidyl choline (egg-PC) were purchased from Avanti Polar Lipids (Alabaster, Ala.). Sodium cholate, cholesterol, cholesteryl oleate, hydrogen peroxide and other chemicals were purchased from Sigma Chemical Company (St. Louis, Mo.).

GF9- and TREM-1 TRIOPEP-containing discoidal SLP. Discoidal TREM-1/TRIOPEP-containing SLP (GF9- and TREM-1/TRIOPEP-dSLP) were prepared using a general procedure described elsewhere (see e.g., Rojas, et al. Biochim Biophys Acta 2018, 1864:2761-2768; Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; Sigalov US 20130045161, and Sigalov US 20110256224, each of which is herein incorporated by referene in it's entirety).

In one embodiment, to prepare GF9-dSLP that contain GF9 and an equimolar mixture of oxidized apo A-I peptides PA22 and PE22, the molar ratio was 65:25:3:1:190, corresponding to POPC:POPG:GF9:apo A-I:sodium cholate. POPC and POPG in organic solvents were mixed, dried in a stream of argon, and placed under vacuum for 8 h.

In one embodiment, to prepare TRIOPEPdSLP, the molar ratio was 28:12:1 for DMPC:DMPG:TREM-1/TRIOPEP. DMPC and DMPG in organic solvents were mixed, dried in a stream of argon, and placed under vacuum for 8 h. In one embodiment, the molar ratio was 65:25:3 for POPC:POPG:TREM-1/TRIOPEP. POPC and POPG in organic solvents were mixed, dried in a stream of argon, and placed under vacuum for 8 h. To synthesize fluorescently labeled nanoparticles, rhodamine B-PE in chloroform was also added to a lipid mixture. To synthesize Gd-labeled nanoparticles, 14:0 PE-DTPA (Gd) in chloroform was also added to a lipid mixture. Then, lipid films were dispersed in phosphate-buffered saline (PBS), pH 7.4, sonicated for 5 min, and aqueous solution of either oxidized or unmodified PA22 and PE22 or either oxidized or unmodified TREM-1/TRIOPEP GA31 or oxidized or unmodified TREM-1/TRIOPEP GE31 or their equimolar mixture (PA22:PE22=1:1, PA/E22; or GA31:GE31=1:1, GA/E31) was added. Amount of GF9 or TREM-1/TRIOPEP was controllably varied in different preparations. Then, the mixture was incubated for 3 h at 30° C. The same procedure was used to prepare dSLP with GF9-G control peptide or either oxidized or unmodified TREM-1/TRIOPEPs GA31-A and GE31-A.

GF9 and TREM-1 TRIOPEP-containing spherical SLP. Spherical GF9- or TREM-1/TRIOPEP-containing SLP (TREM-1/TRIOPEP-sSLP) were prepared using a general sodium cholate dialysis procedure described elsewhere (see e.g., Rojas, et al. Biochim Biophys Acta 2018, 1864:2761-2768; Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; Sigalov US 20130045161, and Sigalov US 20130045161 and US 20110256224, each of which is herein incorporated by referene in it's entirety).

In one embodiment, to prepare GF9-sSLP that contain GF9 and an equimolar mixture of oxidized apo A-I peptides PA22 and PE22 (PA/E22), the molar ratio was 125:6:2:3:1:210, corresponding to POPC:cholesterol:cholesteryl oleate:GF9:apo A-I:sodium cholate. POPC, cholesterol, and cholesteryl oleate in organic solvents were mixed, dried in a stream of argon, and placed under vacuum for 8 h.

In one embodiment, the molar ratio was 60:3:1:1:103 for egg-PC:cholesterol:cholesteryl oleate:TREM-1/TRIOPEP:sodium cholate. Egg-PC, cholesterol, and cholesteryl oleate in organic solvents were mixed, dried in a stream of argon, and placed under vacuum for 8 h. In one embodiment, the molar ratio was 125:6:2:3:1:210 for POPC: cholesterol:cholesteryl oleate:TREM-1/TRIOPEP:sodium cholate. POPC, cholesterol, and cholesteryl oleate in organic solvents were mixed, dried in a stream of argon, and placed under vacuum for 8 h. To synthesize fluorescently labeled nanoparticles, rhodamine B-PE in chloroform was also added to a lipid mixture. To synthesize Gd-labeled nanoparticles, 14:0 PE-DTPA (Gd) in chloroform was also added to a lipid mixture. Then, lipid films were dispersed in Tris-buffered saline-EDTA (TBS-EDTA, pH 7.4), sonicated for 5 min and incubated for 30 min at 30° C. To the dispersed lipids, aqueous solution of either oxidized or unmodified PA22 and PE22 or either oxidized or unmodified TREM-1/TRIOPEP GA31 or oxidized or unmodified TREM-1/TRIOPEP GE31 or their equimolar mixture (PA22:PE22=1:1, PA/E22; or GA31:GE31=1:1, GA/E31) was added. Amount of GF9 or TREM-1/TRIOPEP is controllably varied in different preparations. Then, sodium cholate solution was added and the mixture was incubated at 30° C. for 3 h, followed by extensive dialysis against PBS to remove sodium cholate. The same procedure was used to prepare sSLP with GF9-G control peptide or either oxidized or unmodified TREM-1/TRIOPEPs GA31-A and GE31-A.

Purification and characterization of discoidal and spherical GF9- and TREM-1 TRIOPEP-containing SLP. The obtained TREM-1/TRIOPEP-SLP particles were purified on a calibrated Superdex 200 HR gel filtration column (GE Healthcare Biosciences, Pittsburgh, Pa.) using the BioCAD 700E Workstation (Applied Biosystems, Carlsbad, Calif.) and characterized by analytical RP-HPLC and non-denaturing gel electrophoresis. Peptide concentrations in the GF9-SLP and TREM-1/TRIOPEP-SLP particles were measured as described in Sigalov and Stern. Chem Phys Lipids 2001, 113:133-146. Final peptide compositions were determined in the prepared particles by analytical RP-HPLC essentially as previously described in Sigalov and Stern. Chem Phys Lipids 2001, 113:133-146. Total cholesterol was determined enzymatically using a Boehringer-Mannheim kit and the manufacturer's suggested procedure. Phospholipids were determined by a phosphorus assay. The mean size of the particles was determined using electron microscopy (EM) essentially as described in Sigalov and Stern. Chem Phys Lipids 2001, 113:133-146 and Sigalov. Contrast Media Mol Imaging 2014, 9:372-382. Briefly, the GF9-SLP and TREM-1/TRIOPEP-SLP complexes (at a concentration of about 0.3 mg of GF9 or TRIOPEP/ml) were extensively dialyzed against 5 mM ammonium bicarbonate, mixed with the same volume of 2% phosphotungstate, pH 7.4, and examined using a FEI Tecnai 12 Spirit BioTwin transmission electron microscope (FEI Company, Hillsboro, Oreg.) at 80 KV accelerating voltage on carbon-coated Formvar grids. Microphotographs were photographed at an instrument magnification of 87000× and 92000×, and mean particle dimensions of 100 particles were determined from each negative.

This example demonstrates that SLP are self-assembled upon binding of trifunctional peptides and compounds to lipid and lipid mixtures. This example further demonstrates that depending on method of preparation and composition of lipid mixtures, SLP of discoidal or spherical morphology can be prepared. This example further demonstrates that SLP of discoidal or spherical morphology that contain different TREM-1 inhibitors including, but not limited to, TREM-1 inhibitory peptides GF9, GA31 and/or GE31 can be prepared depending on the TREM-1 inhibitor used. This example further demonstrates that SLP of discoidal or spherical morphology that contain different imaging probes including, but not limited to, Gd-based contrast agents (GBCA) or rhodamine B fluorescent label can be prepared depending on the imaging probe-conjugated lipids used.

### Example 3: In Vitro Macrophage Endocytosis of Fluorescent Synthetic Lipopeptide Particles

In vitro studies of macrophage endocytosis of fluorescent discoidal or spherical GF9-SLP or TREM-1/TRIOPEP-SLP were performed using the standard methods well known in the art (see e.g. Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; Sigalov US 20130045161; and Sigalov US 20110256224, each of which is herein incorporated by referene in it's entirety).

The BALB/c murine macrophage cell line J774A.1 (ATCC TIB-67) were obtained from the American Type Culture Collection (ATCC, Manassas, Va.). The macrophage cells were cultured at 37° C. with 5% CO₂ in Dulbecco's modified Eagle's medium (DMEM) (Cellgro, Mediatech Inc, Manassas, Va.) with 2 mM glutamine, 100 U/ml penicillin, 0.1 mg/ml streptomycin, and 10% fetal bovine serum (FBS) (Cellgro, Mediatech Inc, Manassas, Va.) and grown to approximately 90% of confluence in 6-well tissue culture plates (Corning, Tewksbury, Mass.). Cells were incubated for varied time periods from 4 to 24 h at 37° C. with fluorescently labeled SLP containing GF9 and an equimolar mixture of PA22 and PE22 in either oxidized or unmodified form (GF9-SLP) or TREM-1/TRIOPEP in either oxidized or unmodified form at a concentration of 4 μM rhodamine B (rho-B). After incubation, cells were washed twice with PBS and lysed using Promega passive lysis buffer (Promega, Madison, Wis.). The rhodamine B fluorescence was measured in the lysates with a 540 nm excitation and a 590 nm emission filters using the Gemini EM fluorescence microplate reader (Molecular Devices, Sunnyvale, Calif.). The protein concentration in the lysates was determined using the Bradford reagent (Bio-Rad, Richmond, Calif.) and the SpectraMax 190 microplate reader (Molecular Devices, Sunnyvale, Calif.).

As described in (Sigalov 2014, Sigalov 2014, Shen et al. 2015, Shen and Sigalov 2017, Shen and Sigalov 2017, Rojas et al. 2018, Tornai et al. 2019) and disclosed in US 20130045161 and US 20110256224, endocytosis of SLP with oxidized methionine residues is significantly higher as compared to their unmodified counterparts.

This example demonstrates that sulfoxidation of methionine residues in PA22 and PE22 or in TREM-1/TRIOPEP targets discoidal and spherical GF9-SLP and TREM-1/TRIOPEP-SLP to macrophages and enhances in vitro macrophage endocytosis of these TREM-1/TRIOPEP-SLP. See FIG. 7A. This example further demonstrates that depending on morphology of GF9-SLP and TREM-1/TRIOPEP-SLP, different kinetic parameters of the endocytosis can be observed for SLP of discoidal or spherical morphology. See FIG. 7B.

### Example 4: Immunofluorescence Analysis of TREM-1 and GF9 or TREM-1/TRIOPEP (GE31) in the Cell Membrane

Immunofluorescence analysis of TREM-1 and TREM-1/TRIOPEP in the cell membrane was performed using the standard, well-known in the art methods as described in Shen and Sigalov J Cell Mol Med 2017, 21:2524-2534, and Rojas et al. 2018, herein incorporate by reference.

BALB/c murine macrophage J774A.1 cells were grown at 37° C. in six-well tissue culture plates containing glass coverslips. After reaching target confluency of approximately 50%, cells were incubated for 6 h at 37° C. with Dylight 488-labeled GF9 or TREM-1/TRIOPEP-sSLP that contained Dylight 488-labeled GE31. TREM-1 staining was performed was performed using an Alexa 647-labeled rat anti-mouse TREM-1 antibody (Bio-Rad, Hercules, Calif.). ProLong Gold Antifade DAPI (4′,6′-diamidino-2-phenylindole) mounting medium was used to mount coverslips, and the slides were photographed using an Olympus BX60 fluorescence microscope. Confocal imaging was performed with a Leica TCS SP5 II laser scanning confocal microscope.

This example demonstrates that free GF9 self-inserts into the cell membrane from the outside the cell and colocalizes with TREM-1. The example further demonstrates that upon endocytosis by macrophages, TREM-1/TRIOPEP is released by SLP, self-inserts into the cell membrane and colocalizes with TREM-1. See FIGS. 6A-C.

### Example 5: In Vitro Cytokine Release

In vitro studies of cytokine release by lipopolysaccharide (LPS)-stimulated macrophages in the presence of free GF9 or GF9-G or discoidal and spherical TREM-1/TRIOPEP-SLP containing TREM-1/TRIOPEP in either oxidized or unmodified form or TREM-1/TRIOPEP in free form were performed using the standard, well-known in the art methods as described in Sigalov. Int Immunopharmacol 2014, 21:208-219, herein incorporated by referene in it's entirety.

The BALB/c murine macrophage cell line J774A.1 (ATCC TIB-67) were obtained from the American Type Culture Collection (ATCC, Manassas, Va.). Macrophages were cultured in 48-well plates (Corning, Cambridge, Mass.) for 24 h at 37° C. in the presence of LPS (1 μg/ml, Escherichia coli 055:B5, Sigma) in combination with 50 ng/ml GF9, control peptide GF9-G, control peptide TRIOPEP-A or TREM-1/TRIOPEP in either free or SLP-bound form. Cell-free supernatants were harvested and stored at −20° C. for later cytokine quantification. TNF-alpha, IL-6, and IL-1beta were assayed using commercial ELISA kits (Pierce Biotechnology, Thermo Scientific, Rockford, Ill.) according to the recommendations of the manufacturer. Results were represented as the mean±S.D. of three independent experiments. Statistical significances in in vitro macrophage uptake assay were determined by two-tailed Student's t test.

This example demonstrates that in contrast to control peptide GF9-G, GF9 or the TREM-1 inhibitory GF9 sequence (Domain A) in TREM-1/TRIOPEP in free or SLP-bound form inhibits production of cytokines by LPS-stimulated macrophages. This example further demonstrates that substitution of Lys₅ of TREM-1/TRIOPEP with Ala₅ in TREM-1/TRIOPEP-A results in the loss of cytokine production-inhibiting activity. See FIG. 7A-B and FIG. 10.

### Example 6: Mouse Model of LPS-Induced Endotoxemia and In Vivo Survival and Cytokine Release Studies

Animal survival studies and studies of in vivo cytokine release were performed in a mouse model of LPS-induced septic shock using the standard, well known in the art methods as described in Sigalov. Int Immunopharmacol 2014, 21:208-219.

Naïve, female C57BL/6 mice at 8 to 10 weeks of age (18 to 21 g) from the Jackson Laboratory were randomly grouped (10 mice per group) and i.p. injected with vehicle or the indicated doses of dexamethasone (DEX), control peptide GF9-G, GF9, control peptide TRIOPEP-A or TREM-1/TRIOPEP in either free or SLP-bound form. One hour later, mice received i.p. injection of 30 mg/kg LPS from E. coli 055:B5 (Sigma). In some experiments, all formulations were i.p. administered 1 and 3 h after LPS injection. The viability of mice was examined hourly. Body weights were measured daily. In all of the animal experiments, blood samples were collected via a sub-mandibular (cheek) bleed at 90 min after administration of LPS. Statistical analysis of survival curves was performed by the Kaplan-Meier test. Comparisons were made using two-tailed Student's t test. The production of cytokines in serum was measured by a standard sandwich cytokine ELISA procedure using TNF-alpha, IL-1beta and IL-6 ELISA kits (Pierce Biotechnology, Thermo Scientific, Rockford, Ill.) according to the instructions of the manufacturer. Statistical significances in cytokine analysis ELISA data were determined by two-tailed Student's t test.

This example demonstrates that GF9 and TREM-1/TRIOPEP in free or SLP-bound form inhibit LPS-stimulated cytokine production in vivo. This example further demonstrates that GF9 and TREM-1/TRIOPEP in free or SLP-bound form protect mice from LPS-induced septic shock and prolongs survival of septic mice. This example further demonstrates that the magnitude of this effect can depend on dose and administration time schedule and whether GF9 and TREM-1/TRIOPEP are administered in free or SLP-bound form. See FIGS. 18A-D.

### Example 7: Lung Cancer Tumor Xenografts in Nude Mice and In Vivo Tumor Growth Studies

Animal efficacy studies were performed in human xenograft mouse models of NSCLC using female 6-8 week old NU/J mice from the Jackson Laboratory (Bar Harbor, Me.) using the standard, well-known in the art methods as described in Sigalov. Int Immunopharmacol 2014, 21:208-219 and disclosed in Wu, et al. U.S. Pat. No. 8,415,453 and Sigalov U.S. Pat. No. 8,513,185, each of which is herein incorporated by reference in it's entirety.

Animal efficacy studies were performed using female 6-8 week old NU/J mice from the Jackson Laboratory (Bar Harbor, Me.). Animals were handled as specified in the USDA Animal Welfare Act (9 CFR, Parts 1, 2, and 3) and as described in the Guide for the Care and Use of Laboratory Animals from the National Research Council. Human lung carcinoma cell lines H292 and A549 were obtained from ATCC. Tumor cells in culture were harvested and resuspended in a 1:1 ratio of RPMI 1640 and Matrigel (BD Biosciences, San Jose, Calif.). NSCLC xenografts were established by injecting subcutaneously into the right flanks 5×10⁶ viable cells per mouse. Tumor volumes were calculated with caliper measurements using the formula V=π/6 (length×width×width). When tumor volumes reached an average of 200 mm³, tumor-bearing animals were randomized into groups of 10, and dosing of GF9 or TREM-1/TRIOPEP in free or SLP-bound form was initiated. All tested formulations were intraperitoneally (i.p.) injected at indicated doses and administration schedule. Clinical observations, body weights and tumor volume measurements were made 3 times weekly. Tumor volumes were analyzed using repeated measures ANOVA followed by Bonferroni test. Data points were represented as mean tumor volume±SEM. Antitumor effects were expressed as the percentage of T/C (treated versus control), dividing the tumor volumes from treatment groups with the control groups and multiplied by 100. According to the National Cancer Institute (NCI) standards (see e.g., Johnson, et al. Br J Cancer 2001, 84:1424-1431), a % T/C≤42 is indicative of antitumor activity. At the end of the experiment, the animals were sacrificed and the tumors were excised and weighed.

This example demonstrates that GF9 or TREM-1/TRIOPEP in free or SLP-bound form inhibits tumor growth in two human NSCLC xenograft mouse models. This example further demonstrates that the magnitude of an anticancer effect can depend on dose and time schedule for administration and whether TREM-1 inhibitory peptides are administered in free or SLP-bound form. This example further demonstrates that GF9 and TREM-1/TRIOPEP in free or SLP-bound form are non-toxic and well-tolerable by cancer mice. See FIGS. 13-16.

### Example 8: Pancreatic Cancer Tumor Xenografts in Nude Mice and In Vivo Tumor Growth Studies

In order to demonstrate that modulation of the TREM-1/DAP-12 signaling pathway using GF9 and TREM-1 TRIOPEP in free form and bound to SLP is effective in inhibiting TREM-1-mediated cell activation and reducing pancreatic tumor (PC) growth, animal efficacy studies were performed in human xenograft mouse models of PC using 5-6 week old female athymic nude-Foxn1^{nu} mice obtained from Envigo (formerly Harlan, Inc.) using the standard, well known in the art methods as described in Shen and Sigalov. Mol Pharm 2017, 14:4572-4582, herein incorporated by referene in it's entirety.

### Animal Studies

Mice were implanted subcutaneously into the right flank with 5×10⁶ AsPC-1, BxPC-3 or Capan-1 cells in equal parts of serum-free growth medium and Matrigel. Mice were monitored daily and tumor measurements were taken along the length and width using Vernier calipers twice weekly until sacrifice. Tumor volumes were calculated using a modified ellipsoidal formula: (Length×Width²)/2. When tumors reached a calculated volume of approximately 150-200 mm³, mice were sorted into treatment groups and i.p. injected intraperitoneally once daily for 5 days per week (5qw) at indicated doses. Treatment persisted for 31 days, 29 days and 29 days for mice containing established AsPC-1, BxPC-3 and Capan-1 xenograft tumors, respectively. Mice were humanely sacrificed when individual tumors exceeded 1500 mm³.

### Immunohistochemistry

All staining and quantification procedures were performed by HistoTox Laboratories. Briefly, mice containing AsPC-1, BxPC-3, and Capan-1 tumors were humanely euthanized for necropsy at the end of the study. Excised tumors were fixed using 10% neutral buffered formalin for 1-2 days, processed for paraffin embedding, and sectioned at 4 m. Antigen retrieval for F4/80 was achieved using Proteinase K (Dako North America). Sections were blocked for peroxidase and alkaline phosphatase activity using Dual Endogenous Enzyme Block (Dako North America). Sections were then incubated with Protein Block (Dako North America) followed by primary antibody F4/80 (1:2000, AbD Serotec) diluted using 1% bovine serum albumin in Tris-buffered saline. Afterward, sections were incubated using EnVision+ secondary antibodies (Dako North America), followed by 3,3′-diaminobenzidine in chromogen solution (Dako North America) and counterstained using hematoxylin (Dako North America). Quantitative analysis of intratumoral F4/80 staining was determined using Visiopharm software.

### Cytokine Detection

Blood was collected on study days 1 and 8 and processed into serum. Serum cytokines were analyzed by Quantibody Mouse Cytokine Array Q1 kits (RayBiotech) according to the manufacturer's instructions. Statistical Analysis. All statistical analyses were performed using GraphPad Prism 6.0 (GraphPad Software). Percent treatment/control (T/C) values were calculated using the following formula: % T/C=100×ΔT/ΔC where T and C are the mean tumor volumes of the drug-treated and control groups, respectively, on the final day of the treatment; ΔT is the mean tumor volume of the drug-treated group on the final day of the treatment minus mean tumor volume of the drug-treated group on initial day of dosing; and ΔC is the mean tumor volume of the control group on the final day of the treatment minus mean tumor volume of the control group on initial day of dosing.

### Statistical Analysis

Results are expressed as the mean±SEM. Statistical differences were analyzed using analysis of variance with Bonferroni adjustment unless otherwise noted. The Kaplan-Meier method was used to estimate survival as a function of time, and survival differences were analyzed by the log-rank test. p values less than 0.05 were considered significant.

This example demonstrates that TREM-1 inhibitory peptide GF9 and TREM-1/TRIOPEP in free or SLP-bound form inhibit tumor growth in three human PC xenograft mouse models. This example further demonstrates that TREM-1 blockade using these formulations improves survival. This example further demonstrates that TREM-1 blockade using these formulations reduces the intratumoral macrophage infiltration and that the magnitude of an anticancer effect can depend on the xenograft and dose and whether GF9 and TREM-1/TRIOPEP are administered in free or SLP-bound form. This example further demonstrates that the anticancer activity of the TREM-1 inhibitory formulations correlates with basal intratumoral macrophage content. The example further demonstrates that TREM-1 blockade using TREM-1 inhibitory peptide GF9 or trifunctional peptides TREM-1/TRIOPEP GA31 and GE31 is accompanied by reduction of serum levels of IL-1α, IL-6 and CSF-1. See FIG. 14B (shown for BxPC-3).

### Example 9: Mouse Tolerability Studies

Mouse tolerability studies were performed in healthy C57BL/6 mice using the standard, well-known in the art methods as described in Sigalov. Int Immunopharmacol 2014, 21:208-219, herein incorporated by reference.

Naïve, female C57BL/6 mice at 8 to 10 weeks of age (18 to 21 g) from the Jackson Laboratory were used. Animals were randomly grouped (5 mice per group) and i.p. injected with increasing doses of GF9 or TREM-1/TRIOPEP in free form. Clinical observations and body weights were made twice daily.

This example demonstrates that TREM-1/TRIOPEP in free form is non-toxic and well tolerated in healthy mice at doses of up to at least 400 mg/kg. This example further demonstrates that GF9 in free form is non-toxic and well tolerated in healthy mice at doses of up to at least 300 mg/kg. See FIG. 16.

### Example 10: Haemodynamic Studies in Septic Rats

The role of GF9 and TREM-1-related trifunctional peptides in further models of septic shock, is investigated by performing LPS- and cecal ligation and puncture (CLP)-induced endotoxinemia experiments in rats. The experiments can be conducted analogously to those described in Gibot, et al. Infect Immun 2006, 74:2823-2830 and disclosed in Faure, et al. U.S. Pat. No. 8,013,116; Faure, et al. U.S. Pat. No. 9,273,111; and Sigalov U.S. Pat. No. 8,513,185, each of which is herein incorporated by reference in it's entirety.

### LPS-Induced Endotoxinemia

Animals are randomly grouped (n=10-20) and treated with Escherichia coli LPS (0111:B4, Sigma-Aldrich, Lyon, France) i.p. in combination with control peptide TREM-1/TRIOPEP-A or TREM-1/TRIOPEP in either free or SLP-bound form at various concentrations.

### CLP Polymicrobial Sepsis Model

Rats (n=6-10 per group) are anesthetized by i.p. administration of ketamine (150 mg/kg). The caecum is exposed through a 3.0-cm abdominal midline incision and subjected to a ligation of the distal half followed by two punctures with a G21 needle. A small amount of stool is expelled from the punctures to ensure potency. The caecum is replaced into the peritoneal cavity and the abdominal incision closed in two layers. After surgery, all rats are injected s.c. with 50 mL/kg of normal saline solution for fluid resuscitation. Control peptide GF9-G, GF9, control peptide TRIOPEP-A or TREM-1/TRIOPEP in either free or SLP-bound form are then administered at various concentrations.

### Haemodynamic Measurements in Rats

Immediately after LPS administration as well as 16 hours after CLP, arterial BP (systolic, diastolic, and mean), heart rate, abdominal aortic blood flow, and mesenteric blood flow are recorded. Briefly, the left carotid artery and the left jugular vein are cannulated with PE-50 tubing. Arterial BP is continuously monitored by a pressure transducer and an amplifier-recorder system (IOX EMKA Technologies, Paris, France). Perivascular probes (Transonic Systems, Ithaca, N.Y.) are wrapped up the upper abdominal aorta and mesenteric artery, allowed to monitor their respective flows by means of a flowmeter (Transonic Systems). After the last measurement (4^{th} hour after LPS and 24^{th} hour after CLP), animals are sacrificed by an overdose of sodium thiopental i.v.

### Biological Measurements

Blood is sequentially withdrawn from the left carotid artery. Arterial lactate concentrations and blood gases analyses are performed on an automatic blood gas analyser (ABL 735, Radiometer, Copenhagen, Denmark). Concentrations of TNF-alpha and IL-1beta in the plasma are determined by an ELISA test (Biosource, Nivelles, Belgium) according to the recommendations of the manufacturer. Plasmatic concentrations of nitrates/nitrites are measured using the Griess reaction (R&D Systems, Abingdon, UK).

### Statistical Analyses

Between-group comparisons are performed using Student's t tests. All statistical analyses are completed with Statview software (Abacus Concepts, Calif.).

### Example 11: Attenuation of Intestinal Inflammation in Animal Models of Colitis

In order to demonstrate that the GF9 and TREM-1-related TRIOPEP formulations are effective in inhibiting TREM-1-mediated cell activation in animal models of colitis, the experiments can be conducted analogously to those described in Schenk, et al. J Clin Invest 2007, 117:3097-3106 and disclosed in Faure, et al. U.S. Pat. No. 8,013,116; Faure, et al. U.S. Pat. No. 9,273,111 and Sigalov U.S. Pat. No. 8,513,185, each of which is herein incorporated by reference in it's entirety.

### Mice

C57BL/6 mice, purchased from Harlan, and C57BL/6 RAG2−/− mice, bred in a specific pathogen-free (SPF) animal facility, are used at 8-12 weeks of age. All experimental mice are kept in micro-isolator cages in laminar flows under SPF conditions.

### Mouse Models of Colitis

For experiments involving the adoptive T cell transfer model, colitis is induced in C57BL/6 RAG2−/− mice by adoptive transfer of sorted CD4+CD45RBhigh T cells. Briefly, CD4+ T cells are isolated from splenocytes from C57BL/6 mice, and after osmotic lysis of erythrocytes, CD4+ T cells are enriched by a negative MACS procedure for CD8alpha and B220 (purified, biotinylated, hybridoma supernatant) using avidin-labeled magnetic beads (Miltenyi Biotec). Subsequently, the CD4+ T cell-enriched fraction is stained and FACS sorted for CD4+(RM4-5; BD Biosciences—Pharmingen), CD45RBhi (16A; BD Biosciences—Pharmingen), and CD25− (PC61; eBioscience) naive T cells. Each C57BL/6 RAG2−/− mouse is injected i.p. with 1×105 syngeneic CD4+CD45RBhighCD25− T cells. Colitic mice are sacrificed and analyzed on day 14 after adoptive transfer.

For experiments involving the dextran sodium sulfate (DSS) colitis model, C57BL/6 mice are given autoclaved tap water containing 3% DSS (DSS salt, reagent grade, mol wt: 36-50 kDa; MP Biomedicals) ad libitum over a 5-day period. The consumption of 3% DSS is measured. DSS is replaced thereafter by normal drinking water for another 4 days. Mice are euthanized and analyzed at the end of the 9-day experimental period.

Treatment with GF9, TREM-1/TRIOPEP and TREM-1/TRIOPEP-SLP

Upon colitis induction, either starting on day 0 or after onset of colitis on day 3 (as indicated), mice are treated with either a control peptide GF9-G, GF9, a control peptide TREM-1/TRIOPEP-A or TREM-1/TRIOPEP in free or SLP-bound form i.p. injected at various concentrations in 200 ul saline.

### Colitis Scoring

At the end of the experiments, the colon length is measured from the end of the cecum to the anus. Fecal samples are tested for occult blood using hemo FEC (Roche) tests (score 0, negative test; 1, positive test and no rectal bleeding; 2, positive test together with visible rectal bleeding). The colon is divided into 2 parts. From each mouse, identical segments from the distal and proximal colon are taken for protein and RNA isolation and histology, and frozen tissue blocks are prepared for subsequent analysis. Histological scoring of paraffin-embedded H&E-stained colonic sections is performed in a blinded fashion independently by 2 pathologists. To assess the histopathological alterations in the distal colon, a scoring system is established using the following parameters: (a) mucin depletion/loss of goblet cells (score from 0 to 3); (b) crypt abscesses (score from 0 to 3); (c) epithelial erosion (score from 0 to 1); (d) hyperemia (score from 0 to 2); (e) cellular infiltration (score from 0 to 3); and (f) thickness of colonic mucosa (score from 1 to 3). These individual histology scores are added to obtain the final histopathology score for each colon (0, no alterations; 15, most severe signs of colitis).

### RNA Isolation and RT-PCR

RNA is isolated from intestinal tissue samples preserved in RNAlater (QIAGEN), using the RNAeasy Mini Kit (QIAGEN). RT-PCR is performed with 400 ng RNA each, using the TaqMan Gold RT-PCR Kit (Applied Biosystems). Primers are designed as follows: mouse TREM-1, forward 5′-GAGCTTGAAGGATGAGGAAGGC-3′ and reverse 5′-CAGAGTCTGTCACTTGAAGGTCAGTC-3′; mouse TNF, forward 5′-GTAGCCCACGTCGTAGCAAA-3′ and reverse 5′-ACGGCAGAGAGGAGGTTGAC-3′; mouse beta-actin, forward 5′-TGGAATCCTGTGGCATCCATGAAAC-3′ and reverse 5′-TAAAACGCAGCTCAGTAACAGTCCG-3′; human TREM-1, forward 5′-CTTGGTGGTGACCAAGGGTTTTTC-3′ and reverse 5′-ACACCGGAACCCTGATGATATCTGTC-3′; human TNF, forward 5′-GCCCATGTTGTAGCAAACCC-3′ and reverse 5′-TAGTCGGGCCGATTGATCTC-3′; human GAPDH, forward 5′-TTCACCACCATGGAGAAGGC-3′ and reverse 5′-GGCATGGACTGTGGTCATGA-3′. PCR products are semiquantitatively analyzed on agarose gels.

Human TREM-1 and mouse TREM-1 and TNF expression is also assessed by real-time PCR using the TREM-1 QuantiTect primer assay system and QuantiTect SYBR green PCR Kit (both from QIAGEN). GAPDH is used to normalize TREM-1 and TNF expression levels. DNA is amplified on a 7500 Real-Time PCR system (Applied Biosystems), and the increase in gene expression is calculated using Sequence Detection System software (Applied Biosystems).

### Western Blot Analysis

Protein samples are separated on a denaturing 12% acrylamide gel, followed by transfer to nitrocellulose filter and probing with the primary antibody. Anti-TREM-1 (polyclonal goat IgG, 0.1 ug/ml; R&D Systems) or anti-tubulin (clone B-5-1-2, 1:5,000; Sigma-Aldrich) is used as primary reagent. As secondary antibodies, HRP-labeled donkey anti-goat Ig (1:2,000; The Binding Site) and goat anti-mouse Ig (1:4,000; Sigma-Aldrich) are used. Binding is detected by chemiluminescence using a Super Signal West Pico Kit (Pierce).

### Statistics

The unpaired 2-tailed Student t test is used to compare groups; P values less than 0.05 are considered significant.

### Example 12: Autophage Activity and Colitis in Mice

In order to further demonstrate that the GF9 and TREM-1-related TRIOPEP formulations are effective in inhibiting TREM-1-mediated cell activation in animal models of colitis, the experiments can be conducted analogously to those described in Kokten, et al. J Crohns Colitis 2018, 12:230-244 and disclosed in Faure, et al. U.S. Pat. No. 8,013,116; and Faure, et al. U.S. Pat. No. 9,273,111, each of which is herein incorporated by reference in it's entirety.

### Animals

In vivo experiments are performed as recommended by the US National Committee on Ethics Reflection Experiment [described in the Guide for Care and Use of Laboratory Animals, NIH, MD, 1985]. The experiments are performed on 25 adult male C57BL/6 mice [Janvier Labs, Le Genest-Saint-Isle, France] and 10 adult male Trem-1 knock-out [TREM-1 KO] C57BL/6 mice [INSERM U1116, Inotrem Laboratory, Nancy, France], all aged between 7 and 9 weeks. The animals are housed at 22-23° C., with a 12 h/12 h light/dark cycle, and ad libitum access to food and water.

Induction of colitis, treatment with GF9 and TREM-1/TRIOPEP and assessment of disease activity index. Colitis is induced by administration of 3% dextran sulfate sodium [DSS, molecular weight 36,000-50,000, MP Biomedical, Strasbourg, France] dissolved in water for 5 days. DSS is replaced thereafter by normal drinking water for another 5 days. Either a control peptide GF9-G, GF9, a control peptide TREM-1/TRIOPEP-A or TREM-1/TRIOPEP in free or SLP-bound form or the vehicle alone, used as control, are i.p. administered 2 days before colitis induction and then once daily until the last day of DSS administration, at different concentrations in 200 μL of saline. This dose is chosen after having performed dose-response experiments. Bodyweight, physical condition, stool consistency, water/food consumption and the presence of gross and occult blood in excreta and at the anus are determined daily. The DAI is also calculated daily by scoring bodyweight loss, stool consistency and blood in the stool on a 0 to 4 scale. 41 The overall index corresponds to the weight loss, stool consistency and rectal bleeding scores divided by three, and thus ranges from 0 to 4.

### Collection of Colon Tissue and Fecal Samples

Ten days after the initiation of colitis with DSS, the mice are sacrificed by decapitation. The colon is quickly removed, opened along its length and gently washed in PBS [2.7 mmol/L KCl, 140 mmol/L NaCl, 6.8 mmol/L Na2HPO4.2H2O, 1.5 mmol/L KH2PO4, pH 7.4]. For histological assessment samples are fixed overnight at 4° C. in 4% paraformaldehyde solution and embedded in paraffin. For protein extractions samples are frozen in liquid nitrogen [−196° C.] and stored at −80° C. For the gut microbiota analysis, whole fecal pellets are collected daily in sterile tubes and immediately frozen at −80° C. until analysis.

### Histological Assessment and Scoring

Colitis is histologically assessed on 5 m sections stained with hematoxylin-eosin-saffron [HES] stain. The histological colitis score is calculated blindly by an expert pathologist.

### Endoscopic Assessment and Scoring

Endoscopy is performed on the last day of the study, just before the mice are sacrificed. Prior to the endoscopic procedure, mice are anaesthetized by isoflurane inhalation. The distal colon [3 cm] and the rectum are examined using a rigid Storz Hopkins II miniendoscope [length: 30 cm; diameter: 2 mm; Storz, Tuttlingen, Germany] coupled to a basic Coloview system [with a xenon 175 light source and an Endovision SLB Telecam; Storz]. Air is insufflated via a 9-French gauge over-tube and a custom, low-pressure pump with manual flow regulation [Rena Air 200; Rena, Meythet, France]. All images are displayed on a computer monitor and recorded with video capture software [Studio Movie Board Plus from Pinnacle, Menlo Park, Calif.]. The endoscopy score is calculated from three subscores: the vascular pattern [scored from 1 to 3], bleeding [scored from 1 to 4] and erosions/ulcers [scored from 1 to 4].

### Western Blot Analysis

Total protein is extracted from the frozen colon samples by lysing homogenized tissue in a radioimmunoprecipitation assay [RIPA] buffer [0.5% sodium deoxycholate, 0.1% sodium dodecyl sulfate [SDS] and 1% NP-40] supplemented with protease inhibitors [Roche Diagnostics, Mannheim, Germany]. Protein is then quantified using the bicinchoninic acid assay method. For each mouse, a total of 20 μg of protein is transferred to a 0.45 m polyvinylidene fluoride [PVDF] or 0.45 m nitrocellulose membrane following electrophoretic separation on a denaturing acrylamide gel. The membrane is blocked with 5% w/v non-fat powdered milk or 5% w/v bovine serum albumin [BSA] diluted in Tris-buffered saline with 0.1% v/v Tween® 20 [TBST] for 1 h at room temperature. The PVDF or nitrocellulose membranes are then incubated overnight at 4° C. with various primary antibodies diluted in either 5% w/v nonfat powdered milk or 5% w/v BSA, TBST. After washing in TBST, the appropriate HRP-conjugated secondary antibody is added and the membrane is incubated for 1 h at room temperature. After further washing in TBST, the proteins are detected using an ECL or ECL PLUS kit [Amersham, Velizy-Villacoublay, France]. Glyceraldehyde 3-phosphate dehydrogenase [GAPDH] is used as an internal reference control.

Enzyme-Linked Immunosorbent Assay [ELISA] for Analysis of Soluble TREM-1 [sTREM-1]

At the time of animal sacrifice, whole blood from each mouse is collected into heparinized tubes. These tubes are centrifuged at 3,000 g for 10 min at 4° C. to collect the supernatants, which are stored at −80° C. until use. Plasma concentration of sTREM-1 is determined by a sandwich ELISA technique using the Quantikine kit assay [RnD Systems, Minneapolis, Minn., USA] according to the manufacturers' instructions. Briefly, samples are incubated with a monoclonal antibody specific for TREM-1 pre-coated onto the wells of a microplate. Following a wash, to eliminate the unbound substances, an enzyme-linked polyclonal antibody specific for TREM-1 is added to the wells. After washing away the unbound conjugate, a substrate solution is added to the wells. Color development is stopped and optical density of each well is determined within 30 min using a microplate reader [Sunrise, Tecan, Mannedorf, Switzerland] set to 450 nm, with a wavelength correction set to 540 nm. All measurements are performed in duplicate and the sTREM-1 concentration is expressed in pg/ml.

### Reverse Transcription-Quantitative Polymerase Chain Reaction

Total RNA is purified from the frozen colon samples with the RNeasy Lipid Tissue kit following the recommendation of Qiagen [Courtaboeuf, France], which includes treatment with DNase. To check for possible DNA contamination of the RNA samples, reactions are also performed in the absence of Omniscript RT enzyme [Qiagen]. Reverse transcription is performed using PrimeScript™ RT Master Mix [TAKARA Bio, USA] according to the manufacturer's recommendations with 200 ng of RNA in a 10 μL reaction volume. PCR is then carried out from 2 μL of cDNA with SYBR® Premix Ex Taq™ [Tli RNaseH Plus] [TAKARA Bio, USA] according to the manufacturer's recommendations in a 20 μL reaction volume, with reverse and forward primers at a concentration of 0.2 μM. Specific amplifications are performed using the following primers: TREM-1, forward 5′-CTGTGCGTGTTCTTTGTC-3′ and reverse 5′-CTTCCCGTCTGGTAGTCT-3′. Quantification is performed with RNA polymerase II [Pol II] as an internal standard with the following primers: forward 5′-AGCAAGCGGTTCCAGAGAAG-3′ and reverse 5′-TCCCGAACACTGACATATCTCA-3′. Temperature cycling for TREM-1 is 30 s at 95° C. followed by 40 cycles consisting of 95° C. for 5 s and 59° C. for 30 s. Temperature cycling for RNA polymerase II is 30 s at 95° C. followed by 40 cycles consisting of 95° C. for 5 s and 60° C. for 30 s. Results are expressed as arbitrary units by calculating the ratio of crossing points of amplification curves of TREM-1 and internal standard by using the δδCt method.

### Microbiota Analysis

For the pharmacologically [with TREM-1/TRIOPEP treatment] inhibition of TREM-1, total DNA is extracted from three pooled fecal pellets from each group of mice [day 0 to day 10; n=33 samples]. For microbiota analysis by MiSeq sequencing, the V3-V4 region [519F-785R] of the 16S rRNA gene is amplified with the primer pair S-DBact-0341-b-S-17/S-D-Bact-0785-a-A-21.45 The following quality filters are applied: minimum length=300 base pairs [bp], maximum length=600 bp and minimum quality threshold=20. This filtering yields an average [range] of 25600 reads/samples [14,553-35,490] for further analysis. High-quality reads are pooled, checked for chimeras [using uchime46], and grouped into operational taxonomic units [OTUs][based on a 97% similarity threshold] using USEARCH 8.0.47 Singletons and OTUs representing less than 0.02% of the total number of reads are removed, and the phylogenetic affiliation of each OTU is assessed with Ribosomal Database Project's taxonomy48 from the phylum level to the species level. The mean [range] number of detected OTUs per sample is 324 [170-404]. In the experiments involving Trem-1 KO mice, similar methods are applied but total DNA is extracted from individual fecal pellets of each mouse from the four groups of animals at baseline [before DSS treatment] and at day 10 [after DSS treatment] [n=37 samples]. Following MiSeq sequencing of the V3-V4 region of the 16S rRNA gene, yielding 2,143,457 raw reads, quality filtering is applied [minimum length=200 bp, maximum length=600 bp and minimum quality threshold=20] and an average [range] of 11,560 reads/samples [7,560-18,495] is kept for further analysis. The mean [range] number of detected OTUs per sample is 599 [131-798].

### Statistical Analysis

A two-tailed Student t test is used to compare two groups and a one-way analysis of variance [ANOVA] is used to compare three or more groups. Bonferroni or Tamhane post hoc tests are applied, depending on the homogeneity of the variance. The threshold for statistical significance is set to p<0.05. The statistical language R is used for data visualization and to perform abundance-based principal component analysis [PCA] and interclass PCA associated with Monte-Carlo rank testing on the bacterial genera.

### Example 13: Modulation of the TREM-1 Pathway During Severe Hemorrhagic Shock in Rats

In order to demonstrate that the GF9 and TREM-1-related TRIOPEP formulations are effective in inhibiting TREM-1-mediated cell activation and preventing organ dysfunction and improving survival in rats during severe hemorrhagic shock, the experiments can be conducted analogously to those described in Gibot, et al. Shock 2009, 32:633-637 and disclosed in Faure, et al. U.S. Pat. No. 8,013,116; Faure, et al. U.S. Pat. No. 9,273,111, and Sigalov. U.S. Pat. No. 8,513,185.

### Animals

Adult male Wistar rats (250-300 g) are purchased from Charles River Laboratories (Wilmington, Mass., USA). After 1 week of acclimatization, rats are fasted 12 h before the experiments and are allowed free access to water. All the studies described in the succeeding sentences comply with the regulations concerning animal use and care published by the National Institutes of Health.

### GF9 and TREM-1-Related TRIOPEP Formulations

Control peptide GF9-G, GF9, control peptide TREM-1/TRIOPEP-A and TREM-1-related TRIOPEP in free and SLP-bound form are synthesized as described herein.

### Hemorrhagic Shock Model

Hemorrhagic shock is induced by bleeding from a heparinized (10 UI/mL) carotid artery catheter. Briefly, the rats are anesthetized (50 mg/kg pentobarbital sodium, i.p.) and kept on a temperature-controlled surgical board (37° C.). A tracheostomy is performed, and the animals are ventilated supine (tidal volume, 7-8 mL/kg; rodent ventilator no. 683; Harvard Apparatus, Holliston, Mass.) with a fraction of inspired oxygen of 0.3 and a respiratory rate of 60 breaths per minute. Anesthesia and respiratory support are maintained during the whole experiment. The left carotid artery and the left jugular vein are cannulated with PE-50 tubing. Arterial blood pressure is continuously monitored by a pressure transducer and an amplifier-recorder system (IOX EMKA Technologies, Paris, France). After a 30-min stabilization period, blood is drawn in 10 to 15 min via the carotid artery catheter until MAP reached 40 mmHg. Blood is kept at 37° C., and MAP is maintained between 35 and 40 mm Hg during 60 min. Rats are then allocated randomly (n=10-12 per group) to receive 0.1 mL of either saline (isotonic sodium chloride solution), GF9-G, GF9, TREM-1/TRIOPEP-A or TREM-1/TRIOPEP in free or SLP-bound form at various concentrations in 0.1 mL of saline solution over 1 min via the jugular vein (H0). Shed blood and ringer lactate (volume=3× shed volume) are then infused via the jugular vein in 60 min, and rats are observed for a 4-h period before being killed by pentobarbital sodium overdose. Killing occurs earlier if MAP decreased to less than 35 mm Hg.

### Arterial Blood Gas, Lactate, and Cytokines

Arterial blood gas and lactate concentrations are determined hourly on an automatic blood gas analyzer (ABL 735; Radiometer, Copenhagen, Denmark). Concentrations of TNF-alpha and IL-6 and sTREM-1 in the plasma are determined in triplicate by enzyme-linked immunosorbent assay (Biosources, Nivelles, Belgium; RnD Systems, Lille, France).

### Bacterial Translocation

Rats are killed under anesthesia, and mesenteric lymph node (MLN) complex, spleen, and blood are aseptically removed 4 h after the beginning of reperfusion (or earlier if MAP decreased <35 mm Hg). Homogenates of MLN and spleen and serial blood dilutions are plated and incubated overnight at 37° C. on Columbia blood agar plates (in carbon dioxide and anaerobically) and Macconkey agar (in air). Visible colonies are then counted.

### Pulmonary Integrity

Additional groups of rats (n=4) are subjected to the same procedure but are also infused via the tail vein with fluorescein isothiocyanate (FITC)-albumin (5 mg/kg in 0.3 mL of phosphate-buffered saline) 2 h after the beginning of reperfusion. Rats in these groups are killed 2 h later with an overdose of sodium pentobarbital (200 mg/kg). Immediately thereafter, the lungs are lavaged three times with 1 mL of phosphate-buffered saline, and blood is collected by cardiac puncture. The bronchoalveolar lavage fluid (BALF) is pooled, and plasma is collected. Fluorescein isothiocyanate-albumin concentrations in BALF and plasma are determined fluorometrically (excitation, 494 nm; emission, 520 nm). The BALF-plasma fluorescence ratio is calculated and used as a measure of damage to pulmonary alveolar endothelial/epithelial integrity as previously described (Yang et al. Crit Care Med 2004; 32:1453-9).

### Statistical Analysis

Data are analyzed using ANOVA or ANOVA for repeated measures when appropriate, followed by Newman-Keuls post hoc test. Survival curves are compared using the log-rank test. A two-tailed value of P less than 0.05 is deemed significant. All analyses are performed with GraphPad Prism software (GraphPad, San Diego, Calif.).

### Example 14: Pharmacological Inhibition of TREM-1 in Experimental Atherosclerosis

In order to further demonstrate that GF9 and the TREM-1-related TRIOPEP formulations are effective in inhibiting TREM-1-mediated cell activation in animal models of atherosclerosis, the experiments can be conducted analogously to those described in Joffre, et al. J Am Coll Cardiol 2016, 68:2776-2793 and disclosed in Faure, et al. U.S. Pat. No. 8,013,116 and Faure, et al. U.S. Pat. No. 9,273,111.

### Animals

Trem-1^{-/-} mice (null for the Trem-1 gene) are generated (GenOway, Lyon, France) and backcrossed for more than 10 generations into a C57BL/6J background. Ten-week-old male C57BL/6J Ldlr^{-/-} mice are subjected to medullar aplasia by lethal total body irradiation (9.5 Gy). The mice are repopulated with an intravenous injection of bone marrow cells isolated from femurs and tibias of sex-matched C57BL/6J Trem-1^{-/-} mice or Trem-1^{+/+} littermates. After 4 weeks of recovery, mice are fed a proatherogenic diet containing 15% fat, 1.25% cholesterol, and 0% cholate for 4, 8, or 14 weeks. Eight-week old male ApoE^{-/-} mice are blindly randomized and treated daily by i.p. injection of GF9-G, GF9, TREM-1/TRIOPEP-A or TREM-1/TRIOPEP in free or SLP-bound form at various concentrations during 4 weeks and were put on either a chow or a high-fat diet (15% fat, 1.25% cholesterol).

### Extent and Compositions of Atherosclerotic Lesions

Plasma cholesterol is measured using a commercial cholesterol kit. The basal half of the ventricles and ascending aorta are perfusion-fixed in situ with 4% paraformaldehyde. Afterward, they are removed, transferred to a phosphate-buffered saline (PBS)-30% sucrose solution, embedded in frozen optimal cutting temperature compound and stored at −70° C. Serial 10-μm sections of the aortic sinus with valves (80 per mouse) are cut on a cryostat. One of every 5 sections is kept for plaque size quantification after Oil Red O (Sigma-Aldrich, St. Louis, Mo.) staining. Thus, 16 sections, spanning an 800-μm length of the aortic root, are used to determine mean lesion area for each mouse. Oil Red O-positive lipid contents are quantified by a blinded operator using HistoLab software (Microvisions Instruments, Paris France), which is also used for morphometric studies. En face quantification is used for atherosclerotic plaques along the thoracoabdominal aorta. The aorta is flushed with PBS through the left ventricle and removed from the root to the iliac bifurcation. Then, the aorta is fixed with 10% neutral-buffered formalin. After a thorough washing, adventitial tissue is removed, and the aorta opened longitudinally to expose the luminal surface. Afterward, the aorta, as one tissue example, is stained with Oil Red O for visualizing with the atherosclerotic lesions, as one disease example, quantified by a blinded operator. Collagen is detected using Sirius red stain, and necrotic core is quantified after Masson's trichrome staining. Macrophage presence is determined using specific antibodies. At least 4 sections per mouse are examined for each immunostaining, and appropriate negative controls are used. For immunostaining of mouse atherosclerotic plaques, as one example of mouse tissue, antibodies against Trem-1 (Bs 4886R), macrophage/monocyte antibody (MOMA)-2 (specifically MAB1852), Ly6G, (1A8), and CD3 (A0452) are used. Terminal dUTP nick end-labeling (TUNEL) staining is performed using histochemistry and fluorescent staining. Total proteins are extracted from human atherosclerotic plaque, as one tissue example, and TREM-1 protein level is quantified by Luminex (Thermo Fischer Scientific).

Cells are cultured in RPMI 1640 medium supplemented with L-alanyl-L-glutamine dipeptide (Glutamax, Thermo Fisher Scientific), 10% fetal calf serum, 0.02 mM b-mercaptoethanol, and antibiotics. For cytokine measurements, splenocytes are stimulated with lipopolysaccharide (LPS) (10 μg/ml) and interferon (IFN)-gamma (100 UI/ml) for 24 or 48 h. IL-10, IL-12, and TNF-a production in the supernatants is measured using specific enzyme-linked immunosorbent assays (ELISA).

Primary macrophages are derived from mouse bone marrow-derived cells (BMDM). Tibias and femurs of C57B16/J male mice are dissected, and their marrow is flushed out. Cells are grown for 7 days at 37° C. in a solution of RPMI 1640 medium, 20% neonatal calf serum, and 20% macrophage-colony-stimulating factor-rich L929-conditioned medium. To analyze oxidized LDL (oxLDL) uptake, BMDMs are exposed to human oxLDL (25 μg/ml) for 24 and 48 h. Cells are washed, fixed, and stained using Red Oil. Foam cells are quantified blindly on 6 to 8 fields, and the mean is recorded. To analyze macrophage phenotype, BMDMs are stimulated with LPS (10 μg/ml) and IFN-g (100 UI/ml) for 24 h. IL-10, IL-12, IL-1b, and TNF-α production in the supernatant is measured using ELISA. To analyze apoptosis susceptibility, macrophages are incubated with TNF-a (10 ng/ml) and cycloheximide (10 μmol/l) for 6 h or etoposide (50 μmol/l) for 12 h, or in a fetal calf serum-free medium. Apoptosis is determined by independent experiments using Annexin V fluorescein isothiocyanate apoptosis detection kit with 7-AAD (APC, BD Biosciences, San Jose, Calif.) according to the manufacturer's instructions.

Human monocytes are isolated using anti-CD14 microbeads from healthy donors. Cells are cultured with macrophage colony-stimulating factor (50 ng/ml) for 7 days to induce mature macrophages. Nonclassical monocytes are labeled in vivo by retro-orbital intravenous injection of 1 mm fluorescent microsphere diluted to one-quarter in sterile PBS. Chimeric Ldlr^{-/-} [symbol] mice were euthanized 48 h later, and cell labeling is checked by flow cytometry. Beads that reflect monocyte recruitment are quantified in 8 aortic sinus sections per mouse.

### Statistical Analysis

Values are mean±SE of the mean. Differences between values are examined using the nonparametric Mann-Whitney U test and are considered significant at a p value of <0.05.

This example demonstrates that TREM-1/TRIOPEP in free form is non-toxic and well tolerated in healthy mice at doses of up to at least 400 mg/kg. See FIG. 18.

### Example 15: Modulation of the TREM-1 Pathway in a Mouse Model of DSS-Induced Colitis and Colitis-Associated Tumorigenesis

In order to demonstrate that modulation of the TREM-1/DAP-12 signaling pathway using GF9 and the TREM-1-related TRIOPEP formulations are effective in inhibiting TREM-1-mediated cell activation, decreasing intestinal epithelial proliferation in dextran sulfate sodium (DSS)-induced colitis and ameliorating the development of inflammation and tumor within the colon through exerting anti-inflammatory effects, the experiments can be conducted analogously to those described in Zhou, et al. Int Immunopharmacol 2013, 17:155-161.

### GF9 and TREM-1-Related TRIOPEP Formulations

GF9, GF9-G, control peptide TREM-1-related TRIOPEP and TRIOPEP-A in free and SLP-bound form are synthesized as described herein.

### Animals and DSS-Induced Colitis and Colitis-Associated Tumorigenesis

C57BL/6 mice are purchased from Zhejiang Provincial Laboratories and (aged 8 to 12 weeks) maintained in a specific pathogen-free facility. Mice are treated with 7 days of 3.5% DSS (MP Biomedicals) in regular drinking water. To develop colitis-associated tumors, mice are first injected with 10 mg/kg azoxymethane (AOM) (Sigma-Aldrich) intraperitoneally (i.p.) followed 5 days later by a 5 day course of 2% DSS. Mice are then allowed to recover for 16 days with regular drinking water. The cycle of five days of 2% DSS followed by 16 days of regular drinking water is repeated twice. Mice are sacrificed 21 days after the last cycle of DSS for tumor counting. Colons are harvested, flushed of feces and longitudinally slit open to grossly count tumors with the aid of a magnifier and stereomicroscope.

### Treatments

Starting on day 0 (at the beginning of colitis induction), mice are treated once daily with GF9, GF9-G, TREM-1/TRIOPEP-A or TREM-1/TRIOPEP in free or SLP-bound form at various concentrations injected i.p. in 200 μl saline. To investigate the effects of blocking TREM-1 after induced inflammation, colitis is induced by 4% DSS for 4 days. After colitis induction, mice are administered with GF9, GF9-G, TREM-1/TRIOPEP-A or TREM-1/TRIOPEP in free or SLP-bound form for the next 5 days.

### Quantitative RT-PCR

Total RNA from colons is collected after colon tissue homogenization using the Trizol (Pierce). cDNA is synthesized using iScript (MBI) and then used in quantitative PCR reactions with SYBR Green using specific primers: TNF-alpha forward 5′-AGGCTGCCC CGACTACGT-3′ and reverse 5′-GACTTTCTCCTGGTATGAGATAGCAAA-3′; IFN-gamma forward 5′-CAGCAACAGCAAGGCGAAA-3′ and reverse 5′-CTGGACCTGTGGGTTGTT GAC-3′; IL-1beta forward 5′-TCGCTCAGGGTCACAAGAAA-3′ and reverse 5′-CATCAGAGGCAAGGAGGAAAAC-3′; IL-6 forward 5′-ACAAGTCGGAGGCTTAATTACACAT-3′ and reverse 5′-ATGTGTAATTAAGCCTCCGACTTGT-3′; IL-17 forward 5′-GCTCCAGAA GGCCCTCAGA-3′ and reverse 5′-AGCTTTCCCTCCGCATTGA-3′; macrophage inflammatory protein-2 (MIP-2) forward 5′-CACTCTCAAGGGCGGTCAA-3′ and reverse 5′-AGGCACATCAGGTACGATCCA-3′; 3-actin forward 5′-AGATTACTGCTCTGGCTC CTA-3′ and reverse 5′-CAAAGAAAGGGT GTAAAACG-3′. Relative expression levels of mRNA are normalized to β-actin. PCR products are separated on a 1.5% agarose gel and stained with ethidium bromide. Relative quantification of mRNA is performed by densitometry using QuantityOne software (e.g. Biorad Laboratories). Reactions are performed on the ABI 7900HT.

### ELISA

The serum levels of TNF-alpha, IL-1beta and IL-6 are measured using the specific ELISA kits (e.g. R&D Systems) following the manufacturer's instructions. All samples are ran in duplicate and analyzed on the same day.

### Evaluation of Inflammation

Colons are harvested from mice, flushed free of feces and jelly-rolled for formalin fixation and paraffin embedding. 5 m sections are used for hematoxylin and eosin staining. Histologic assessment is performed in a blinded fashion using a scoring system. A 3-4 point scale is used to denote the severity of inflammation (0=none, 1=mild, 2=moderate, and 3=severe), the level of involvement (0=none, 1=mucosa, 2=mucosa and submucosa and 3=transmural) and extent of epithelial/crypt damage (0=none, 1=basal 1/3, 2=basal 2/3, 3=crypt loss, and 4=crypt and surface epithelial destruction). Each parameter is then multiplied by a factor reflecting the percentage of the colon involved (0-25%, 26-50%, 51-75%, and 76-100%), and then summed to obtain the overall score. Assessment of colon weight after DSS treatment is performed by measuring the weight of colons (excluding the cecum) after removal of feces and normalizing by the length of colon in age- and sex-matched mice.

### Intestinal Permeability

Mice are fasted for 4 h with the exception of drinking water prior to the administration of 0.6 mg/kg FITC-dextran (4 kD, Sigma). Serum is collected 4 h later retro-orbitally, diluted 1:3 in PBS and the amount of fluorescence is measured using a fluorescent spectrophotometer with emission at 488 nm, and absorption at 525 nm.

### Intestinal Epithelial Proliferation

Mice are injected with 100 mg/kg BrdU (e.g. B.D. Pharmingen) i.p. 2.5 h prior to sacrifice at various time points after treatment with AOM/DSS. Colons are then dissected free, flushed free of feces, jelly-rolled, formalin-fixed, and paraffin-embedded. Sections are subsequently stained using the BrdU (e.g. BD Biosciences).

### Apoptosis.

Colon sections from formalin-fixed, paraffin-embedded tissues are assessed for apoptotic cells using the ApoAlert DNA fragmentation assay kit (e.g. Clontech).

### Statistics

Data are presented as mean±SEM. Survival curves is assessed by log-rank test. The tumor counts, intestinal permeability, cytokine measurements, proliferation and apoptosis levels between mice treated with GF9, GF9-G, TRIOPEP-A or TREM-1/TRIOPEP in free or SLP-bound form are compared using the Student's unpaired t-test. p<0.05 is considered statistically significant.

TREM-1 inhibition by treatment with GF9 and TREM-1/TRIOPEP but not GF9-G or TRIOPEP-A in free and SLP-bound form is anticipated to ameliorate the development of inflammation and tumor within the colon through exerting anti-inflammatory effects. In addition, this treatment is anticipated to decrease intestinal epithelial proliferation in DSS-induced colitis.

### Example 16: Synthesis and Modification of Paclitaxel-Conjugated Peptides in Free and SLP-Bound Form

This example demonstrates one embodiment of a synthesized trifunctional peptide compound containing PTX (PTX/TRIOPEP).

The first step is to synthesize the trifunctional compound comprising domains A and B where domain A is paclitaxel (PTX) bound to TREM-1 inhibitory peptide sequence GFLSKSLVF, whereas domain B is a 22 amino acids-long apolipoprotein A-I helix 6 peptide sequence with either unmodified or modified amino acid residue(s) (see TABLE 2). Although it is not necessary to understand the mechanism of an invention, it is believed that as an anticancer agent, PTX may exhibit not only its microtubule-stabilizing activity, but also its ability to stimulate release of anticancer cytokines from tumor-associated macrophages (TAMs) and functions to treat and/or prevent a cancer-related disease or condition, whereas a 22 amino acids-long apolipoprotein A-I helix 6 peptide sequence with either unmodified or modified amino acid residue(s) functions to assist in the self-assembly of SLP upon binding to lipid or lipid mixtures and to target the particles to cancer cells and/or TAMs, respectively.

In one embodiment, the trifunctional peptide compound comprises domains A and B where domain A is PTX is conjugated to TREM-1 inhibitory peptide sequence GFLSKSLVF, whereas domain B is a 22 amino acids-long apolipoprotein A-I helix 4 peptide sequence with either unmodified or sulfoxidized methionine residue (see TABLE 2).

In one embodiment, PTX is conjugated to the acetylated 31 amino acids-long sequence of TREM-1/TRIOPEP where the domain A comprises acetylated peptide sequence GFLSKSLVF whereas domain B comprises an apolipoprotein A-I helix 6 peptide sequence (i.e. PTX-Gly-Phe-Leu-Ser-Lys-Ser-Leu-Val-Phe-Pro-Leu-Gly-Glu-Glu-Met-Arg-Asp-Arg-Ala-Arg-Ala-His-Val-Asp-Ala-Leu-Arg-Thr-His-Leu-Ala-OH or PTX-GFLSKSLVFPLGEEMRDRARAHVDALRTHLA), hereafter referred to as a PTX/TREM-1-related “TRIOPEP” peptide compound or “PTX-TREM-1/TRIOPEP”.

Peptides can be synthesized or purchased from specialized companies (i.e., Sigma-Genosys, Woodlands, Tex., USA) with greater than 95% purity as assessed by HPLC. Peptide molecular mass can be checked by matrix-assisted laser desorption ionization mass spectrometry. The trifunctional peptide compounds containing conjugated PTX can be synthesized analogously as described in Lin et al. Chem Commun (Cambridge) 2013; 49:4968-4970 and disclosed in Castaigne, et al. U.S. Pat. No. 9,173,891.

### Synthesis of 4-(Pyridin-2-Yldisulfanyl) Butyric Acid

4-Bromobutyric acid (2 g, 12 mmol) and thiourea (0.96 g, 12.6 mmol) are dissolved in ethanol (50 mL) and refluxed at 90° C. for 4 h. After dropwise addition of a NaOH solution (4.8 g in 5:1 H2O/ethanol), the mixture is refluxed for another 16 h and then cooled to room temperature. The white precipitate is collected and redissolved in water (40 mL). 4 M HCl is used to adjust the solution pH to 5, and the product is extracted into diethyl ether. The organic phase is dried over anhydrous sodium sulfate to give 4-sulfanylbutyric acid as a colorless oil (310 mg, 15%), which is used in the next step without further purification. 4-sulfanylbutyric acid (105 mg, 0.87 mmol) and 2-aldrithiol (440 mg, 2.0 mmol, 2.3 eq) are dissolved in MeOH (1.3 mL) and stirred for 3 h. The solution is purified by RP-HPLC (5% to 95% of acetonitrile in water with 0.1% TFA over 45 min), combining product fractions and removing solvents to give 4-(pyridin-2-yldisulfanyl) butyric acid as an oil (118 mg, 59%).

### Paclitaxel C2′ Ester Synthesis

Paclitaxel (186 mg, 0.22 mmol), 4-(pyridin-2-yldisulfanyl)butyric acid (100 mg, 0.44 mmol), N,N′-diisopropylcarbodiimide (DIC) (68 μL, 0.44 mol), and 4-dimethylaminopyridine (DMAP) (26.7 mg, 0.22 mmol) are added into an oven dried flask equipped with a stirrer bar, evacuated and refilled with nitrogen three times to remove air, then dissolved in anhydrous acetonitrile (12.7 mL). The reaction is allowed to stir in the dark at room temperature for 48 h. The solvents are removed under vacuum and the residue is dissolved in chloroform and purified by flash chromatography (3:2 EtOAc/hexane), to give the product as a white solid (108 mg, 47%).

### Synthesis of PTX-TREM-1/TRIOPEP in Free and SLP-Bound Form

GFLSKSLVFPLGEEMRDRARAHVDALRTHLA (89.8 mg, 25.7 umol) and paclitaxel C2′ ester (54.7 mg, 51.4 umol) are added to an oven dried flask equipped with a stirrer bar and evacuated and filled with nitrogen three times to remove the air. The reagents are then dissolved in anhydrous dimethyl formamide DMF (5 mL). The solution is allowed to stir for 16 h, before purification by RP-HPLC (30% to 95% acetonitrile in water with 0.1% TFA over 45 min). Product fractions are combined and lyophilized to give a PTX-TREM-1/TRIOPEP as a white powder. Discoidal and spherical PTX-TREM-1/TRIOPEP-containing SLP are prepared, purified and characterized using the methods and procedures described herein in the Example 2.

### Example 17: Use of PTX-TREM-1/TRIOPEP in Experimental Cancer

In order to demonstrate the anticancer activity of PTX-TREM-1/TRIOPEP, the experiments can be conducted analogously to those disclosed herein and described in Lin et al. Chem Commun (Cambridge) 2013; 49:4968-4970; Sigalov. Int Immunopharmacol 2014, 21:208-219; Shen and Sigalov. Mol Pharm 2017, 14:4572-4582; and disclosed in Castaigne, et al. U.S. Pat. No. 9,173,891.

### Cytotoxicity

The methyl thiazol tetrazolium (MTT) assay can be used to assess the cytotoxic effect of the PTX-TREM-1/TRIOPEP formulations in free or SLP-bound form on cancer cells. The PTX-TREM-1/TRIOPEP formulations may contain either unmodified or modified amino acid residue(s). Briefly, cells are plated in 96-well plates (5000 cells/well) in their respective media. Next day, the monolayers are washed with PBS (pH 7.4) twice, and then incubated at 37° C. for 24 h with the PTX-TREM-1/TRIOPEP formulations in free or SLP-bound form in serum-free media. The following day, 25 μl of MTT (1 mg/ml) is added to each well and incubated for 3 h at 37° C. Plates are centrifuged at 1200 rpm for 5 min. The medium is removed, the precipitates are dissolved in 200 μl of DMSO and the samples are read at 540 nm in a microtiter plate reader.

### Animal Toxicity

Female C57BL6 mice (6-8 weeks, 18-21 g) can be used in toxicity studies of PTX-TREM-1/TRIOPEP formulations in free or SLP-bound form. PTX-TREM-1/TRIOPEP formulations may contain either unmodified or oxidized methionine residue. Groups of six mice each receives injections of 1.5 ml of PBS via the intraperitoneal route, containing respective doses of 30 mg/kg and 40 mg/kg of Taxol®, 40 mg/kg and 70 mg/kg of Abraxane® and different doses of PTX-TREM-1/TRIOPEP in free or SLP-bound form. The injections are administered on days 1, 2 and 3. A control group is injected with the vehicle. The weights and the health of the mice are monitored for 30 days. Weight measurements are performed once a day for the first 7 days and twice a week for the remaining monitoring period.

### Screening for PTX-TREM-1/TRIOPEP Incorporation

Cultured cells are incubated with PTX-TREM-1/TRIOPEP formulations in free or SLP-bound form, labeled with ¹⁴C-PTX. Subsequent to the incubation period, cells are trypsinized and the radioactivity of the lysate is determined to measure the extent of incorporation of the PTX into the cells.

### Tumor Suppression

Tumor suppression studies using PTX-TREM-1/TRIOPEP formulations in free or SLP-bound form can be performed in animal models of cancer similarly as described herein (see e.g., the examples 7 and 8). Female 6-8 week old NU/J mice can be obtained from the Jackson Laboratory (Bar Harbor, Me.) Human cancer cell lines including but not limited to human carcinoma, human pancreas or human breast cancer cell lines can be obtained from ATCC. Tumor cells in culture can be harvested and resuspended in a 1:1 ratio of RPMI 1640 and Matrigel (BD Biosciences, San Jose, Calif.). Human cancer xenografts are established by injecting subcutaneously into the right flanks certain amounts of viable cells per mouse. Tumor volumes are calculated with caliper measurements using the formula V=π/6 (length×width×width). When tumor grows to approximately 125 mm³ (100-150 mm³), animals are pair-matched by tumor size into treatment and control groups. Either PTX (TAXOL®; 30 mg/kg PTX) or PTX-TREM-1/TRIOPEP formulations in free (60 mg/kg PTX) or SLP-bound (30 mg/kg PTX) are intravenously administered to the animals via tail vein. Clinical observations, body weights and tumor volume measurements are made twice a week once tumors become measureable. It should be noted that TAXOL® is formulated with a detergent Cremophor that in itself is cytotoxic and is also the source of numerous side effects during chemotherapy. The Cremophor content of TAXOL® is about 80× that of paclitaxel per ml.

TREM-1 inhibition by treatment with PTX-TREM-1/TRIOPEP in free and SLP-bound form is anticipated to have a significantly higher anticancer activity in terms of tumor inhibition and survival rate improvement as compared to PTX. In addition, this treatment is anticipated to be substantially better tolerated by cancer mice as compared to PTX.

### Example 18: Modulation of the TREM-1 Pathway in Experimental Arthritis

In order to demonstrate that GF9 and TREM-1-related TRIOPEP formulations are effective in inhibiting TREM-1-mediated cell activation and protecting against bone and cartilage damage in animal models of rheumatoid arthritis (RA), the experiments were conducted as described in Shen and Sigalov. J Cell Mol Med 2017, 21:2524-2534.

### Chemicals, Lipids and Cells

Sodium cholate, cholesteryl oleate and other chemicals were purchased from Sigma-Aldrich Company (St. Louis, Mo., USA). 1,2-dimyristoyl-sn-glycero-3-phosphocholine (DMPC), 1,2-dimyristoyl-sn-glycero-3-phospho-(10-rac-glycerol) (DMPG), 1-palmitoyl-2-oleoyl-sn-glycero-3-phosphocholine (POPC), 1-palmitoyl-2-oleoyl-sn-glycero-3-phospho-(10-rac-glycerol) (POPG), 1,2-dimyristoyl-sn-glycero-3-phosphoethanolamine-N-(lissamine rhodamine B sulfonyl) (Rho B-PE) and cholesterol were purchased from Avanti Polar Lipids (Alabaster, Ala., USA). The murine macrophage cell line J774A.1 was obtained from the American Type Culture Collection (ATCC, Manassas, Va., USA).

### Peptide Synthesis

GF9 and two 31-mer methionine-sulfoxidized peptides, GFLSKSLVFPYLDDFQKKWQEEM(O)ELYRQKVE (GE31) and GFLSKSLVFPLGEEM(O)RDRARAHVDALRTHLA (GA31) were ordered from American Peptide Company (Sunnyvale, Calif., USA). All peptides were purified by reversed-phase high-performance liquid chromatography (RP-HPLC), and their purity was confirmed by amino acid analysis and mass spectrometry.

### Synthetic Lipopeptide Particles (SLP)

Discoidal SLP (dSLP) complexes that contain GF9 or an equimolar mixture of TREM-1/TRIOPEP peptides GA31 and GE31 (TREM-1/TRIOPEP) were synthesized as described in Shen and Sigalov. J Cell Mol Med 2017, 21:2524-2534 and herein (see the Example 2). The molar ratio was 65:25:1:190 corresponding to DMPC:DMPG:GA/E31:sodium cholate for GA/E31-dHDL that contain an equimolar mixture of oxidized TREM-1/TRIOPEP peptides GA31 and GE31. Spherical SLP (sSLP) complexes that contain an equimolar mixture of GA31 and GE31 (TREM-1/TRIOPEP-sSLP) were synthesized as described in Shen and Sigalov. J Cell Mol Med 2017, 21:2524-2534 and herein (see the Example 2). The molar ratio was 125:6:2:1:210 corresponding to POPC:cholesterol:cholesteryl oleate:GA/E31-I:sodium cholate for TREM-1/TRIOPEP-sSLP that contain an equimolar mixture of oxidized peptides GA31 and GE31. All obtained SLP formulations were purified and characterized as described in Shen and Sigalov. J Cell Mol Med 2017, 21:2524-2534 and herein (see the Example 2).

### Animals

All animal experiments were performed in strict accordance with the recommendations in the Guide for the Care and Use of Laboratory Animals of the National Institutes of Health (NIH) and in the United States Department of Agriculture (USDA) Animal Welfare Act (9 CFR, Parts 1, 2, and 3).

### Collagen-Induced Arthritis (CIA) Model

Animal studies were performed by Bolder BioPATH (Boulder, Colo., USA). CIA was induced in male 6- to 7-week-old DBA/1 mice by immunization with bovine type II collagen. Briefly, mice were injected intradermally with 100 μl of Freund's complete adjuvant containing 250 μg of bovine type II collagen (2 mg/ml final concentration) at the base of the tail on day 0 and again on day 21. On day 24, mice were randomized by body weight into treatment groups. At enrolment on day 24, the mean mouse weight was 20 g. Arthritis onset occurred on days 26-38. Starting day 24, mice were injected i.p. intraperitoneally daily for 14 consecutive days with GF9, GF9-dSLP, GF9-sSLP, TREM-1/TRIOPEP-dSLP (dose equivalent to 5 mg of GF9/kg), TREM-1/TRIOPEP-sSLP (dose equivalent to 5 mg of GF9/kg) or with PBS. Mice were weighed on study days 24, 26, 28, 30, 32, 34, 36 and 38 (prior to necropsy). Daily clinical scores were given on a scale of 0-5 for each of the paws on days 24-38. On day 38, mice were killed for necropsy.

### Histology Assessment of Joints

At the end of study, fore paws, hind paws and knees were harvested, fixed in 10% neutral buffered formalin for 1-2 days, and then decalcified in 5% formic acid for 4-5 days before standard processing for paraffin embedding. Sections (8 μm) were cut and stained with toluidine blue (T blue). Hind paws, fore paws and knees were embedded and sectioned in the frontal plane. Six joints from each animal were processed for histopathological evaluation. The joints were then assessed using 0-5 scale for inflammation, pannus formation, cartilage damage, bone resorption and periosteal new bone formation. A summed histopathology score (sum of five parameters, 0-25 scale) was also determined.

### Cytokine Detection

Plasma was collected on days 24, 30 and 38, and cytokines were analysed by Quantibody Mouse Cytokine Array Q1 kits (RayBiotech, Norcross, Ga., USA) according to the manufacturer's instructions.

### Statistical Analysis

All statistical analyses were performed with GraphPad Prism 6.0 software (GraphPad, La Jolla, Calif., USA). Results are expressed as the mean±SEM. Statistical differences were analyzed using analysis of variance with Bonferroni adjustment. P values less than 0.05 were considered significant.

This example demonstrates that GF9 or TREM-1/TRIOPEP in free or SLP-bound form ameliorate CIA and protect against bone and cartilage damage. This therapeutic effect is accompanied by a reduction in the plasma levels of macrophage colony-stimulating factor and pro-inflammatory cytokines such as TNF-alpha, interleukin (IL)-1 and IL-6. This example further demonstrates that GF9, GF9-SLP, TREM-1/TRIOPEP-SLP formulations are non-toxic and well-tolerable by arthritic mice. See FIG. 17A-B.

### Example 19: Modulation of the TREM-1 Pathway in Experimental Retinopathy

In order to demonstrate that GF9 and TREM-1-related TRIOPEP formulations are effective in inhibiting TREM-1-mediated cell activation and reducing pathological retinal neovascularization (RNV), the experiments were conducted as described in Rojas, et al. Biochim Biophys Acta 2018, 1864:2761-2768, herein incorporated by reference in it's entirety.

### Synthetic Lipopeptide Particles (SLP)

Spherical SLP that contain GF9 or an equimolar mixture of GA31 and GE31 (TREM-1/TRIOPEP-sSLP) were synthesized using the sodium cholate dialysis procedure, purified and characterized as described herein and in Shen and Sigalov. Mol Pharm 2017, 14:4572-4582 and Shen and Sigalov. J Cell Mol Med 2017, 21:2524-2534. In a subset of experiments, 1,2-dimyristoyl-sn-glycero-3-phosphoethanolamine-N-(lissamine rhodamine B sulfonyl) was added to reaction mixtures to prepare rhodamine B (rho-B)-labeled rho B-labeled TREM-1/TRIOPEP-sSLP as described in Shen and Sigalov. Mol Pharm 2017, 14:4572-4582 and Shen and Sigalov. J Cell Mol Med 2017, 21:2524-2534.

### In Vitro Macrophage Uptake.

BALB/c murine macrophage J774A.1 cells were obtained from ATCC (Manassas, Va.) and cultured according to manufacturer's instructions at 37° C. in 6-well tissue culture plates containing glass coverslips until reaching about 50% confluency. Then, cells were incubated for 6 h at 37° C. either with rho B-labeled GF9-SLP that contained Dylight labeled GF9 or TREM-1/TRIOPEP-sSLP that contained Dylight 488-labeled GE31. In colocalization experiments, TREM-1 staining was performed using an Alexa 647-labeled rat anti-mouse TREM-1 antibody (Bio-Rad, Hercules, Calif.) as described in Shen and Sigalov. J Cell Mol Med 2017, 21:2524-2534. Coverslips were mounted using Prolong Gold anti-fade DAPI (4′,6-diamidino-2-phenylindole) mounting medium and photographed using an Olympus BX60 fluorescence microscope. Confocal imaging was performed using a Leica TCS SP5 II laser scanning confocal microscope as described in Shen and Sigalov. J Cell Mol Med 2017, 21:2524-2534.

### Mouse Model of Oxygen-Induced Retinopathy (OIR)

This study was carried out in strict accordance with the recommendations in the Guide for the Care and Use of Laboratory Animals of the National Institutes of Health and in the United States Department of Agriculture (USDA) Animal Welfare Act (9 CFR, Parts 1, 2, and 3). animals were treated according to the ARVO Statement for the Use of Animals in Ophthalmic and Vision Research.

Litters of C57BL/6J (Jackson Laboratory, Bar Harbor, Me.) neonatal mice and nursing dams were exposed to a hyperoxia environment (75% oxygen) from postnatal day 7 (P7) to P12 and returned to normoxia until P17. The hyperoxia exposure causes degeneration of the immature retinal vessels. This results in severe hypoxia upon return to the normoxia environment which leads to vitreoretinal neovascularization. Beginning on P7, mice were treated until day P17 by daily i.p. injections of GF9, GF9-SLP, TREM-1/TRIOPEP-sSLP or vehicle (phosphate-buffered saline, pH 7.4; PBS). In a subset of experiments, rho B-labeled GF9-sSLP and TREM-1/TRIOPEP-sSLP were used to confirm the ability of these particles to cross the BRB. In another subset of experiments, rho B-labeled Gd-containing sSLP were used to confirm the ability of these targeted SLP to cross the BRB in other species (rats and rabbits). In another subset of experiments, neonatal mice and nursing dams were not subjected to a hyperoxia environment and reared in room air (RA). At P17, all mice were humanely sacrificed and their retinas were collected.

### Immunofluorescence Staining

Treatment effects on vaso-obliteration and pathological angiogenesis were assessed by morphometric analysis of the avascular and neovascularization areas in retinal flat mounts after labeling with isolectin B₄ as described in Patel, et al. Am J Pathol 2014, 184:3040-3051. Immunofluorescence analysis (IFA) of the retina flat mounts was performed to assess the effects of the TREM-1-targeting treatments on the distribution of TREM-1, M-CSF and markers for inflammatory cells (CD45) and activated macrophage/microglial cells (Iba-1) in relation to RNV. Retinal frozen sections from pups kept in RA and from the OIR pups were fixed in 4% paraformaldehyde for 15 min (or in cold acetone at −20° C. for 30 min), washed 3 times with PBS, and blocked with a solution containing 0.3% Triton X and 3% normal goat serum (NGS) for 30 min. Then, the samples were reacted with a rat anti-mouse TREM-1 antibody (Abcam, Cambridge, Mass.), rabbit polyclonal anti-mouse M-CSF antibodies (Abcam, Cambridge, Mass.), rabbit polyclonal anti-mouse CD45 antibodies (Santa Cruz Biotechnology, Dallas, Tex.), a rabbit anti-mouse Iba-1 antibody (Wako Chemical USA, Inc.), and kept at 4° C. overnight. Then, the samples were washed 3 times with PBS and stained with a donkey-anti-rat Oregon green antibody for TREM-1, a goat anti-rabbit Texas red antibody for CD45 and Iba-1 or a donkey anti-rabbit Texas red antibody for M-CSF (Invitrogen, Waltham, Mass.). After washing 3 times with PBS, the images were captured with a 20× lens using a Zeiss Axioplan2 fluorescence microscope (Carl Zeiss Meditec, Inc., Dublin, Calif.). Intravitreal neovascular formation and avascular area were measured as described in Connor, et al. Nat Protoc 2009, 4:1565-1573.

### Western Blot Analysis

Retina samples from OIR-treated and RA control pups were homogenized in the modified RIPA buffer (20 mM Tris-HCl, 2.5 mM EDTA, 50 mM NaF, 10 mM Na₄P₂O₇, 1% Triton X-100, 0.1% sodium dodecyl sulfate, 1% sodium deoxycholate, 1 mM phenyl methyl sulfonyl fluoride, pH 7.4). Samples containing equal amounts of protein were separated by 12% sodium dodecyl sulfate polyacrylamide gel electrophoresis, transferred to nitrocellulose membrane, and reacted for 24 hrs with monoclonal rat anti-mouse TREM-1 or polyclonal rabbit M-CSF antibodies (Abcam, Cambridge, Mass.) in 5% milk, followed by incubation with corresponding horseradish peroxidase-linked secondary antibodies (GE Healthcare Bio-Science Corp., Piscataway, N.J.). Bands were quantified by densitometry, and the data were analyzed using ImageJ software and normalized to loading control. Equal loading was verified by stripping the membranes and reprobing them with a monoclonal antibody against β-actin (Sigma-Aldrich, St Louis, Mo.).

### Statistical Analysis

Group differences were compared by one way ANOVA followed with a post hoc test for multiple comparisons. Values are represented as the means±standard error of the means (SEM). Results were considered statistically significant when P≤0.05.

This example demonstrates that GF9 and TREM-1/TRIOPEP in free and SLP-bound form significantly (up to 95%) reduce pathological RNV in a mouse model of retinopathy. It further that demonstrates that GF9 and TREM-1/TRIOPEP in free and SLP-bound form are non-toxic and well-tolerated in mouse litters. TREM-1 inhibition substantially downregulates retinal protein levels of TREM-1 and M-CSF (CSF-1) suggesting that TREM-1-dependent suppression of pathological angiogenesis involves M-CSF (CSF-1). This example further demonstrates that sSLP, GF9, GF9-SLP and TREM-1/TRIOPEP-sSLP pass the blood-retinal barrier (BRB) and blood-brain barrier (BBB). See FIGS. 18A-D-19.

### Example 20: Modulation of the TREM-1 Pathway in Experimental Alcoholic Liver Disease (ALD)

In order to demonstrate that TREM-1-related TRIOPEP formulations are effective in inhibiting TREM-1-mediated cell activation and ameliorating ALD, the experiments were conducted in the Lieber DeCarli ALD mouse model as described in Tornai et al. Hepatol Commun 2019,3:99-115, and Petrasek, et al. J Clin Invest 2012, 122:3476-3489.

### Reagents and Cells

The murine macrophage J774A.1 cells were purchased from ATCC. Cytochalasin D was purchased from MP Biomedicals (Solon, Ohio, USA). Blocker of lipid transport 1 (BLT-1) was purchased from Calbiochem (Torrey Pines, Calif., USA). Sodium cholate, cholesteryl oleate, fucoidan and other chemicals were purchased from Sigma-Aldrich (St. Louis, Mo., USA). 1-palmitoyl-2-oleoyl-sn-glycero-3-phosphocholine (POPC), 1,2-dimyristoyl-sn-glycero-3-phosphoethanolamine-N-(lissamine rhodamine B sulfonyl) (rho B-PE) and cholesterol were purchased from Avanti Polar Lipids (Alabaster, Ala., USA).

### Peptide Synthesis

The following synthetic peptides were ordered from Bachem (Torrance, Calif., USA): one 9-mer peptide GFLSKSLVF (human TREM-1213-221, GF9), two 22-mer methionine sulfoxidized peptides PYLDDFQKKWQEEM(O)ELYRQKVE (H4) and PLGEEM(O)RDRARAHVDALRTHLA (H6) that correspond to human apo A-I helices 4 (apo A-I123-144) and 6 (apo A-I167-188), respectively, and two 31-mer methionine sulfoxidized peptides, GFLSKSLVFPYLDDFQKKWQEEM(O)ELYRQKVE (GE31) and GFLSKSLVFPLGEEM(O)RDRARAHVDALRTHLA (GA31).

### Synthetic Lipopeptide Particles (SLP)

SLP of spherical morphology that contained either GF9 and an equimolar mixture of PE22 and PA22 (GF9-sSLP) or an equimolar mixture of GA31 and GE31 (TREM-1/TRIOPEP-sSLP) were synthesized using the sodium cholate dialysis procedure, purified and characterized as previously described herein and in Shen and Sigalov. Mol Pharm 2017, 14:4572-4582 and Shen and Sigalov. J Cell Mol Med 2017, 21:2524-2534. For GF9-sSLP, the initial molar ratio was 125:6:2:3:1:210 corresponding to POPC:cholesterol:cholesteryl oleate:GF9:apo A-I:sodium cholate, respectively, where apo A-I is an equimolar mixture of PE22 and PA22. For TREM-1/TRIOPEP-sSLP, the initial molar ration was 125:6:2:1:210 corresponding to POPC:cholesterol:cholesteryl oleate:GA/E31:sodium cholate, where GA/E31 is an equimolar mixture of GA31 and GE31 peptides.

### In Vitro Macrophage Uptake

BALB/c murine macrophage J774A.1 cells (ATCC, Manassas, Va., USA) were cultured at 37° C. with 5% CO2 in Dulbecco's Modification of Eagle's Medium, DMEM (Cellgro Mediatech, Manassas, Va., USA) with 2 mM glutamine, 100 U ml-1 penicillin, 0.1 mg/ml streptomycin and 10% heat inactivated fetal bovine serum (Cellgro Mediatech, Manassas, Va., USA) and grown to approximately 90% confluency in 12 well tissue culture plates (Corning Costar, Corning, N.Y., USA). After reaching target confluency, cells were incubated for 1 h in medium with or without fucoidan (400 μg/mL), BLT-1 (10 μM) or cytochalasin D (40 μM), Cells were subsequently incubated for 4 h and 22 h at 37° C. in medium containing 2 μM of rho B-labeled GF9-sSLP or TREM-1/TRIOPEP-sSLP (as calculated for rho B). Cells were washed twice using PBS and lysed using Passive Lysis Buffer (Promega, Madison, Wis., USA). Rho B fluorescence was measured in the lysates with 544 nm excitation and 590 nm emission filters using a Fluoroscan Ascent CF fluorescence microplate reader (Thermo Labsystems, Vantaa, Finland). The protein concentrations in the lysates were measured using Bradford Reagent (Sigma-Aldrich, St. Louis, Mo., USA) and a MRX microplate reader (Dynex Technologies, Chantilly, Va., USA) according to the manufacturer's recommended protocol.

### Animals

C57BL/6 female mice (10- to 12-week-old) were purchased from The Jackson Laboratory (Bar Harbor, Me., USA) and housed at the University of Massachusetts Medical School (UMMS) animal facility. All animals received humane care in accord with protocols approved by the UMMS Institutional Animal Use and Care Committee. Mice (n=6-9/group) were acclimated to a Lieber-DeCarli liquid diet of 5% ethanol (vol/vol) over a period of 1 week, then maintained on the 5% diet for 4 weeks. Pair-fed control mice were fed a calorie-matched dextran-maltose diet. All animals had unrestricted access to water throughout the entire experimental period. In treated groups, mice were i.p. treated 5 days/week with vehicle (empty sSLP) or the TREM-1 inhibitory formulations GF9-sSLP (2.5 mg of GF9/kg) or TREM-1/TRIOPEP-sSLP (5 mg equivalent of GF9/kg) (SignaBlok, MA, USA), from the first day on a 5% ethanol diet. At the end of all animal experiments, cheek blood samples were collected in serum collection tubes (BD Biosciences, San Jose, Calif., USA) and processed within an hour. After blood collections, mice were euthanized, and liver samples were harvested and stored at −80° C. until further analysis.

Total Protein Isolation from Liver

Total protein was extracted from liver samples using RIPA buffer (Boston Bio-products Cat. #BP-115) supplemented with protease inhibitor cocktail tablets (Roche Cat. #11836153001) and Phospho Stop phosphatase inhibitor (Roche Cat. #04906837001). Cell debris were then removed from cell lysates by 10 minutes centrifugation at 2000 rpm.

### Biochemical Assays and Cytokines

Serum alanine aminotransferase (ALT) levels were determined by kinetic method using commercially available reagents from Teco Diagnostics (Anaheim, Calif., USA). Liver triglycerides were extracted using a 5% NP-40 lysis solution buffer and quantified using a commercially available kit (Wako Chemicals, Richmond, Va., USA) followed normalization to protein amount analyzed by Pierce BCA protein assay (Thermo Scientific, Rockford, Ill., USA). Cytokine levels were measured in serum samples and whole liver lysates diluted in assay diluent following the manufacturer's instructions. Specific anti-mouse ELISA kits were used for the quantification of MCP-1, TNFα (BioLegend Inc., San Diego, Calif., USA) and IL-1β (R&D Systems, Minneapolis, Minn., USA) levels. For normalization, the total protein concentration of the whole liver lysate was determined using Pierce BCA protein assay.

### Western Blot Analysis

Whole liver proteins were boiled in Laemmli's buffer. The samples were resolved in 10% SDS-PAGE gel under reducing conditions using Tris-glycine buffer system and resolved proteins transferred onto a nitrocellulose membrane. SYK proteins were detected by specific primary antibodies (SYK: 2712—Cell Signaling and phospho-SYKY525/526: ab58575—Abcam) followed by an appropriate secondary HIRP-conjugated IgG antibody from Santa Cruz Biotechnology. β-actin, detected by an ab49900 antibody (Abcam), was used as a loading control. The specific immunoreactive bands of interest were visualized by chemiluminescence (Bio-Rad) using the Fujifilm LAS-4000 luminescent image analyzer.

### RNA Extraction and Quantitative Real-Time PCR Analysis

Total RNA was extracted using the Qiagen RNeasy kit (Qiagen) according to the manufacturer's instructions with on-column DNase treatment. RNA was quantified using a Nanodrop 2000 spectrophotometer (Thermo Scientific) and cDNA synthesis was performed using the iScript Reverse Transcription Supermix (Bio-Rad Laboratories) and 1 μg total RNA. Real-time quantitative PCR was performed using Bio-Rad iTaq Universal SYBR Green Supermix (Bio-Rad Laboratories) and a CFX96 real-time detection system (Bio-Rad Laboratories). Relative gene expression was calculated by the comparative ΔΔCt method. The expression level of target genes was normalized to the house-keeping gene, 18S rRNA, in each sample and the fold-change in the target gene expression between experimental groups was expressed as a ratio. Primers were synthesized by IDT, Inc. and the sequences are listed in Table 3A.

TABLE 3A
Mouse Primer Sequences.

| | Primers | |
|---|---|---|
| Mouse | Forward sequence | Reverse sequence |
| primers | 5′ to 3′ | 5′ to 3′ |
| 18s | GTA ACCCGTTGAACC | CCATCCAATCGGTAGT |
| | CCATT | AGCG |
| TREM-1 | TCCTATTACAAGGCTG | AAGACCAGGAGAGGAA |
| | ACAGAGCGTC | ACAACCGC |
| TNF-α | CACCAC CATCAA GG | AGGCAACCTGACCAC |
| | ACTC AA | TCTCC |
| MCP-1 | CAGGTCCCT GTCATG | CAGGTCCCTGTC ATG |
| | CTTCT | CTTCT |
| IL-1β | CTTTGAAGTTGACGGA | TGAGTGATACTGCCTG |
| | CCC | CCTG |
| MPO | CATCCAACCCTTCATG | CTGGCGATTCAGTTTG |
| | TTCC | G |
| LY6G | TGCGTTGCTCTGCTGG | CAGAGTAGTGGGGCAG |
| | AGATAGA | ATGG |
| F4/80 | TGCATCTAGCAATGGA | GCCTTCTGGATCCATT |
| | CAGC | TGAA |
| CD68 | TGTCTGATCTTGCTAG | GAGAGTAACGGCCTTT |
| | GACCG | TTGTG |
| Pro- | GCTCCTCTTAGGGGCC | CCACGTCTCACCATTG |
| Collagen1α | ACT | GG |
| α-SMA | GTCCCAGACATCAGGG | TCGGATACTTCAGCGT |
| | AGTAA | CAGGA |
| ACC1 | AGCAGATCCGCAGCTT | ACCTCTGCTCGCTGAG |
| | G | TGC |
| MIP-1α | TTCTCTGTACCATGAC | GCATTAGCTTCAGATT |
| | ACTCTGC | TACGGGT |
| RANTES | GCTGCTTTGCCTACCT | TCGAGTGACAAACACG |
| | CTCC | ACTGC |
| ADRP | CTGTCTACCAAGCTCT | CGATGCTTCTCTTCCA |
| | GCTC | CTCC |
| PPARα | AACATCGAGTGTCGAA | AGCCGAATAGTTCGCC |
| | TATGTGG | GAAAG |
| SREBF1 | GCTTCTTACAGCACAG | TTTCATGCCCTCCATA |
| | CAACC | GACAC |
| CPT1A | CCAGGCTACAGTGGGA | GAACTTGCCCATGTCC |
| | CATT | TTGT |
| MCAD/ | GATCGCAATGGGTGCT | AGCTGATTGGCAATGT |
| MACD | TTTGATAGAA | CTCCAGCAAA |

### Liver Histopathology

Sections of formalin-fixed, paraffin-embedded liver specimens from mice were stained with Hematoxylin/Eosin (H&E) or F4/80 (ThermoFisher, Cat #MF48000), MPO (Abcam Cat #ab9535) antibodies for immunohistochemistry, the fresh frozen samples were stained with Oil-Red-O at the UMMS DERC histology core facility.

### Statistical Analysis

All statistical analyses were performed using GraphPad Prism 7.02 (GraphPad Software Inc.). Significance levels were determined using one way analysis of variance (ANOVA) followed by a post hoc test for multiple comparisons. Data are shown as mean±SEM and differences were considered statistically significant when P≤0.05. Significance levels were showed using the following symbols: *, 0.05≥P≥0.01; ** 0.01≥P≥0.001; ***, 0.001≥P≥0.0001; ****, P≤0.0001.

This example demonstrates that TREM-1/TRIOPEP in SLP-bound form significantly reduced serum ALT and cytokine protein levels in a mouse model of ALD. It further that demonstrates that TREM-1/TRIOPEP in SLP-bound form are non-toxic and well-tolerated in mice with ALD. TREM-1/TRIOPEP significantly inhibits macrophage (F4/80, CD68) and neutrophil (lymphocyte antigen 6 complex locus G6D and myeloperoxidase, Ly6G and MPO, respectively) markers and proinflammatory cytokines monocyte chemoattractant protein-1, tumor necrosis factor-α, interleukin-1l and macrophage inflammatory protein-1α (MCP-1, TNF-α, IL-1β, MIP-1α, respectively) at the mRNA level as compared to the sSLP vehicle. This example further demonstrates that TREM-1/TRIOPEP-sSLP formulations ameliorates liver steatosis and early fibrosis markers (α-smooth muscle actin, αSMA, and pro-collagen1α) on the mRNA level in alcohol-fed mice. See FIG. 20.

### Example 21: Synthesis of Imaging Probe ([⁶⁴Cu])-Conjugated Peptides in Free and SLP-Bound Form

This example demonstrates one embodiment of a synthesized TREM-1-related trifunctional peptide compound containing imaging probe [⁶⁴Cu] ([⁶⁴Cu]TREM-1/TRIOPEP).

The first step is to synthesize the trifunctional compound comprising domains A and B where domain A is a TREM-1 inhibitory peptide sequence GF9, whereas domain B is either a [⁶⁴Cu]-labeled 22 amino acids-long apolipoprotein A-I helix 6 peptide sequence with sulfoxidized methionine residue or a [⁶⁴Cu]-labeled 22 amino acids-long apolipoprotein A-I helix 4 peptide sequence with sulfoxidized methionine residue. Although it is not necessary to understand the mechanism of an invention, it is believed tha 22 amino acids-long apolipoprotein A-I helix 4 and 6 peptide sequences with sulfoxidized methionine residues will assist in the self-assembly of SLP upon binding to lipid or lipid mixtures and target the [⁶⁴Cu]TREM-1/TRIOPEP-SLP particles to macrophages, whereas GF9 peptide sequence will assist in the self-insertion of [⁶⁴Cu]TREM-1/TRIOPEP released from [⁶⁴Cu]TREM-1/TRIOPEP-SLP particles upon endocytosis by macrophages (e.g., TAMs, Kupffer cells, etc.) into the cell membrane and subsequent colocalization of [⁶⁴Cu]TREM-1/TRIOPEP with TREM-1 expressed on TAMs. This is believed to result in TREM-1 inhibition along with [⁶⁴Cu]TREM-1/TRIOPEP-PET signal in the macrophage-rich areas of interest allowing for visualization of macrophage-mediated inflammation (e.g., neuroinflammation, inflamed atherosclerotic plaques, intratumoral inflammation, etc.).

In one embodiment, [⁶⁴Cu] is conjugated to the 22 amino acids-long sequence of domain B comprising an apolipoprotein A-I helix 6 peptide sequence Ac-Pro-Leu-Gly-Glu-Glu-Met-Arg-Asp-Arg-Ala-Arg-Ala-His-Val-Asp-Ala-Leu-Arg-Thr-His-Leu-Ala-OH (i.e., PTX-PLGEEMRDRARAHVDALRTHLA), hereafter referred to as a [⁶⁴Cu]-related “TRIOPEP” peptide compound or “[⁶⁴Cu]/TRIOPEP”.

Peptides can be synthesized or purchased from specialized companies (i.e., Sigma-Genosys, Woodlands, Tex., USA) with greater than 95% purity as assessed by HPLC. Peptide molecular mass can be checked by matrix-assisted laser desorption ionization mass spectrometry.

The trifunctional peptide compounds containing conjugated [⁶⁴Cu] can be synthesized analogously as disclosed in James and Andreasson, WO 2017083682A1, herein incorporated by reference in its entirety.

DOTA (1,4,7, 10-tetraazacyclododecane-1,4,7,10-tetraacetic acid) conjugation is performed according to established protocols, using metal-free buffers. After conjugation, matrix-assisted laser desorption/ionization (MALDI) mass spectrometry is conducted to determine the average number of DOTA molecules conjugated per TREM-1/TRIOPEP. Subsequently, the DOTA-conjugated TREM-1/TRIOPEP is radiolabeled with [⁶⁴Cu] by incubating it in a [⁶⁴Cu]CuCl₂ solution (pH 5.5) at 37° C. for one hour with continual shaking. The reaction is purified via a NAP5 column and specific activity of the final labeled TREM-1/TRIOPEP is determined via size exclusion HPLC. [⁶⁴Cu]TREM-1/TRIOPEP can be synthesized with high specific radioactivity (>75 GBq/μΓTîoI), radiochemical purity (>99%), and labeling efficiency (50-75%), which is sufficient for in vitro and in vivo use.

Discoidal and spherical [⁶⁴Cu]TREM-1/TRIOPEP-containing SLP are prepared, purified and characterized using the methods and procedures described herein in the Example 2.

### Example 22: Use of [⁶⁴Cu]TREM-1/TRIOPEP in Imaging of Neuroinflammation

In one embodiment, in order to demonstrate the feasibility of using [⁶⁴Cu]TREM-1/TRIOPEP to visualize neuroinflammation in vivo, PET/CT imaging of middle cerebral artery occlusion (MCAo) mice can be performed analogously as disclosed in James and Andreasson, WO 2017083682A1. Discoidal and spherical [⁶⁴Cu]TREM-1/TRIOPEP-containing SLP can be prepared, purified and characterized using the methods and procedures described herein in the Examples 2 and 21.

The MCAo model of cerebral ischemia is selected since the time-course of macrophage infiltration and microglial activation in the brain infarct is well documented, and because this model is commonly used to evaluate candidate microglial/macrophage-PET tracers. B6 mice (n=3), MCAo (n=9), and sham (n=9) mice are injected via tail vein with 80-85μΩ of [⁶⁴Cu]TREM-1/TRIOPEP-containing SLP in a saline solution (0.9% sodium chloride) and imaged using PET/CT at 3 h post-injection. They are imaged again at 19 h post-injection, which is 1.5-2 days after surgery/stroke.

PET signal from brain tumors is anticipated to be significantly higher than observed in healthy brain regions or sham mice. Similarly, biodistribution results are anticipated to reveal significantly higher tracer uptake in the whole brain of GBM mice compared to shams. Autoradiography is anticipated to show markedly higher [⁶⁴Cu]TREM-1/TRIOPEP binding within tumor compared to normal brain and sham brain slices.

### II. Trifunctional Peptides without sHDLs

### Example 1A: Synthesis and Modification of Peptides

In certain embodiments, the ability of resulting amphipathic peptides and compounds of the disclosure to interact with native lipoproteins can be predicted based on their primary amino acid sequences by: 1) the amphipathicity score of these peptide sequences calculated as described above using a variety of computer programs available online (see, for example, http://www.tcdb.org/progs/?tool=pepwheel, http://lbqp.unb.br/NetWheels/, https://npsa-prabi.ibcp.fr/cgi-bin/npsa_automat.pl?page=/NPSA/npsa_amphipaseek.html, http://rzlab.ucr.edu/scripts/wheel/wheel.cgi, http://heliquest.ipmc.cnrs.fr/cgi-bin/ComputParams.py) or other techniques including but not limiting to those described in Jones, et al. J Lipid Res 1992, 33:287-296 and 2) their ability to form SLP structures upon interaction with lipids. While not being bound to any particular theory, it is believed that the amphipathic scores of 5 and higher as calculated for example, using PEPWHEEL (http://www.tcdb.org/progs/?tool=pepwheel) may indicate the ability of the peptides with such scores to interact with native lipoproteins. It is further believed that these peptides can form SLP upon interaction with lipids.

This example demonstrates one embodiment of synthesized TREM-1-related trifunctional peptides (TREM-1/TRIOPEP) G-HV21 and G-KV21.

The first step is to synthesize the 21 amino acids-long peptides comprising domains A and B where domain A is a 9 amino acids-long TREM-1 inhibitory therapeutic peptide sequence, whereas domain B is a 12 amino acids-long amphipathic peptide sequences that contain sulfoxidized methionine residue. Although it is not necessary to understand the mechanism of an invention, it is believed that a 9 amino acids-long TREM-1 inhibitory therapeutic peptide sequence corresponding to a portion of a TREM-1 transmembrane domain sequence affects the TREM-1/DAP-12 receptor complex assembly, inhibits the TREM-1 signaling pathway and functions to treat and/or prevent a TREM-1-related disease or condition, whereas a 12 amino acids-long amphipathic peptide sequences with sulfoxidized methionine residue mediate formation of naturally long half-life LP upon interaction with native lipoproteins in a blood stream in vivo, and target the particles to TREM-1-expressing macrophages and SR-B1-expressing cells (e.g., hepatocytes, cancer cells).

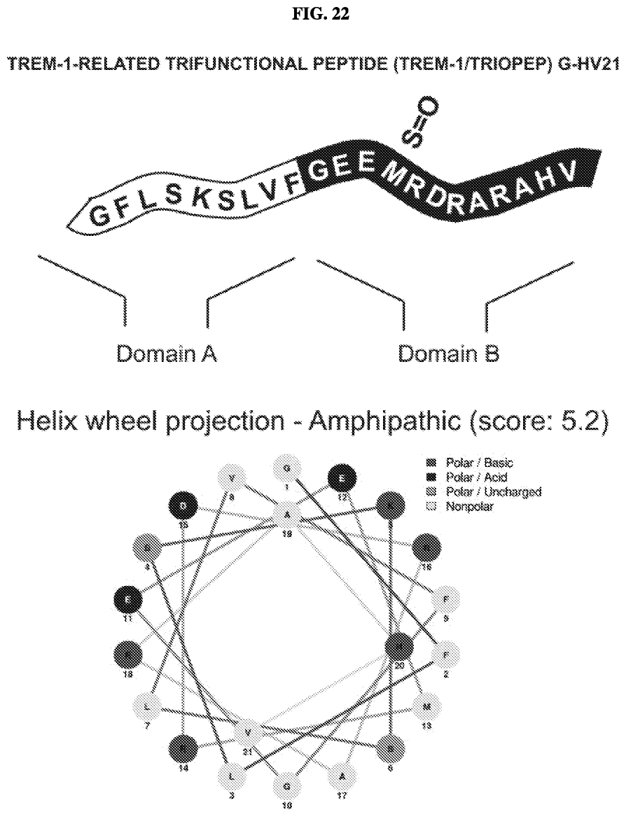

In one embodiment, the amino acid sequence of a resulting trifunctional peptide comprises NH2-Gly-Phe-Leu-Ser-Lys-Ser-Leu-Val-Phe-Gly-Glu-Glu-Met-Arg-Asp-Arg-Ala-Arg-Ala-His-Val-OH (i.e., GFLSKSLVFGEEMRDRARAHV, SEQ ID NO 1), hereafter referred to as a TREM-1-related “TRIOPEP” peptide or “TREM-1/TRIOPEP” G-HV21. See FIG. 22.

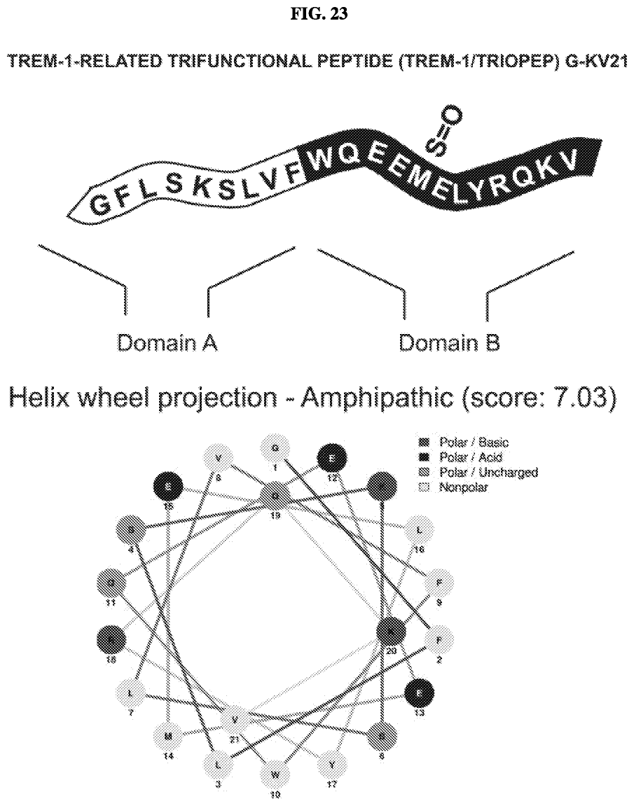

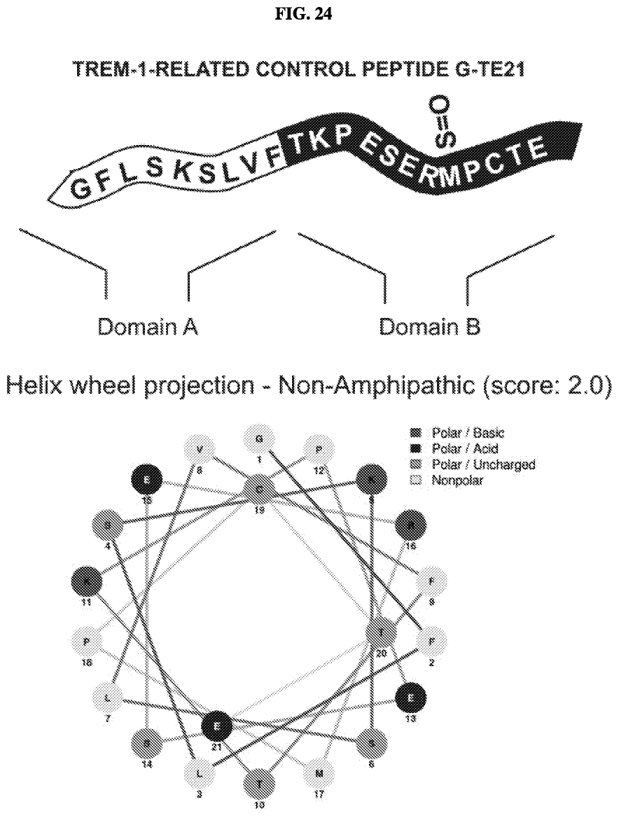

In one embodiment, the amino acid sequence of a trifunctional peptide comprises NH2-Gly-Phe-Leu-Ser-Lys-Ser-Leu-Val-Phe-Trp-Gln-Glu-Glu-Met-Glu-Leu-Tyr-Arg-Gln-Lys-Val-OH (i.e., GFLSKSLVFWQEEMELYRQKV, SEQ ID NO 3), hereafter referred to as a TREM-1-related “TRIOPEP” peptide or “TREM-1/TRIOPEP” G-KV21. See FIG. 23.

In another embodiment, the amino acid sequence of a peptide comprises NH2-Gly-Phe-Leu-Ser-Lys-Ser-Leu-Val-Phe-Thr-Lys-Pro-Glu-Ser-Glu-Arg-Met-Pro-Cys-Thr-Glu-OH (i.e., GFLSKSLVFTKPESERMPCTE), hereafter referred to as a “TREM-1-related control peptide G-TE21” or “G-TE21”. Although it is not necessary to understand the mechanism of an invention, it is believed that this non-amphipathic peptide does not interact with native lipoproteins and therefore, does not form naturally long half-life LP. Thus, G-TE21 may be considered as a “control peptide”.

The example further demonstrates one embodiment of synthesized TCR-related trifunctional peptide (TCR/TRIOPEP) M-VE32.

The first step is to synthesize the 32 amino acids-long peptide comprising domains A and B where domain A is a 10 amino acids-long TCR modulatory therapeutic peptide sequence, whereas domain B is a 22 amino acids-long amphipathic peptide sequences. Although it is not necessary to understand the mechanism of an invention, it is believed that a 10 amino acids-long TCR modulatory therapeutic peptide sequence MF10 corresponding to a portion of a SARS-CoV spike (S) glycoprotein fusion peptide affects the TCR receptor complex assembly, inhibits the TCR signaling pathway and functions to treat and/or prevent a TCR-related disease or condition, whereas a 22 amino acids-long amphipathic peptide sequence mediates formation of naturally long half-life LP upon interaction with native lipoproteins in a bloodstream in vivo, and targets the particles to TCR-expressing cells.

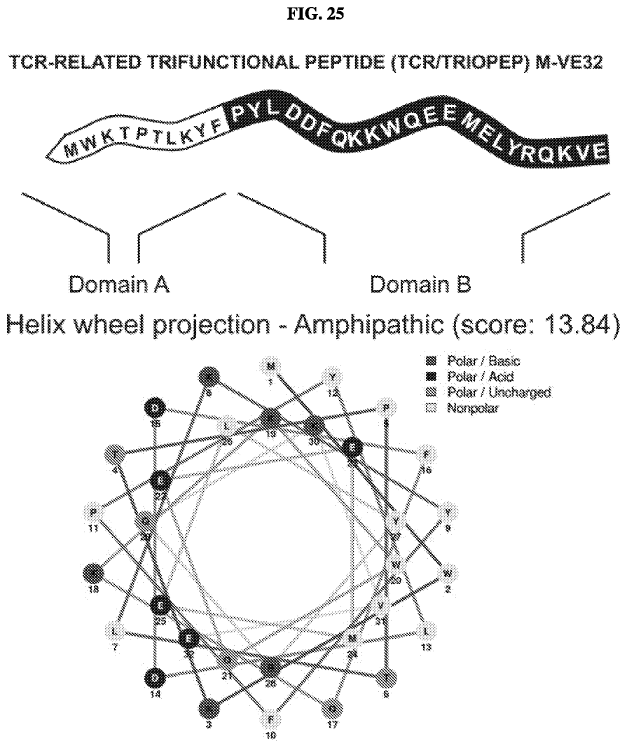

In one embodiment, the amino acid sequence of a resulting trifunctional peptide comprises NH2-Met-Trp-Lys-Thr-Pro-Thr-Leu-Lys-Tyr-Phe-Pro-Tyr-Leu-Asp-Asp-Phe-Gln-Lys-Lys-Trp-Gln-Glu-Glu-Met-Glu-Leu-Tyr-Arg-Gln-Lys-Val-Glu-OH (i.e., MWKTPTLKYFPYLDDFQKKWQEEMELYRQKVE, SEQ ID NO 18), hereafter referred to as a TCR-related “TRIOPEP” peptide or “TCR/TRIOPEP” M-VE32. See FIG. 25.

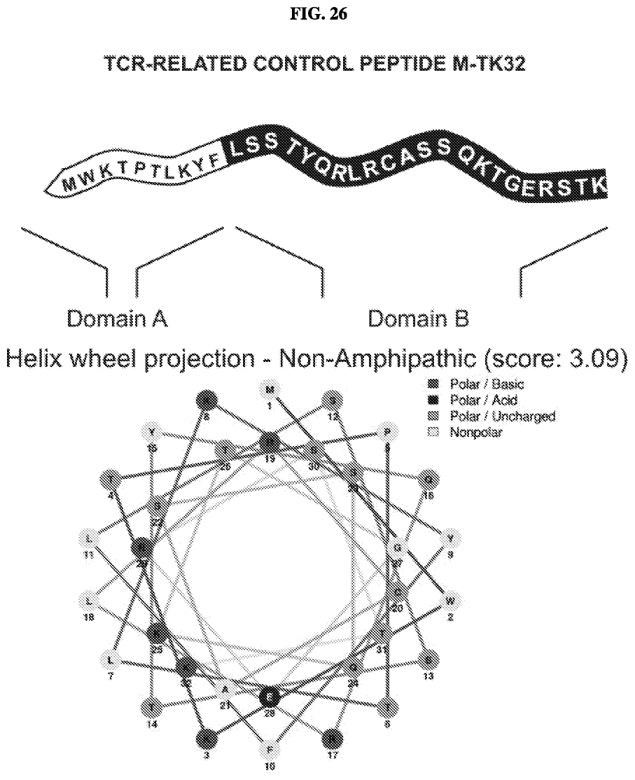

In another embodiment, the amino acid sequence of a peptide comprises NH2-Met-Trp-Lys-Thr-Pro-Thr-Leu-Lys-Tyr-Phe-Leu-Ser-Ser-Thr-Tyr-Gln-Arg-Leu-Arg-Cys-Ala-Ser-Ser-Gln-Lys-Thr-Gly-Glu-Arg-Ser-ThrLys-OH (i.e., MWKTPTLKYFLSSTYQRLRCASSQKTGERSTK), hereafter referred to as a “TCR-related control peptide M-TK32” or “M-TK32”. See FIG. 26. Although it is not necessary to understand the mechanism of an invention, it is believed that this non-amphipathic peptide does not interact with native lipoproteins and therefore, does not form naturally long half-life LP. Thus, M-TK32 may be considered as a “control peptide”.

The peptides can be synthesized using a solid phase peptide synthesis (see e.g., Elmore U.S. Pat. No. 4,749,742). Unprotected unmodified and methionine sulfoxidized peptides can be purchased from specialized companies (i.e., Bachem, Torrance, Calif., USA) with greater than 95% purity as assessed by HPLC. Peptide molecular mass can be checked by matrix-assisted laser desorption ionization mass spectrometry.

To convert methionine residues in unmodified G-HV21, G-KV21, and G-TE21 to methionine sulfoxides, the standard procedure known in the art to prepare protein containing methionine sulfoxides can be also used (see e.g., Elmore U.S. Pat. No. 4,749,742; Sigalov US 20130045161; Sigalov US 20110256224; Sigalov and Stern. FEBS Lett 1998, 433:196-200; Sigalov and Stern. Chem Phys Lipids 2001, 113:133-146). Briefly, a purified peptide (about 15 mg) is dissolved in 1 ml of 3 M guanidine-HCl, pH 7.4, and then hydrogen peroxide is added to a final concentration of 300 mM. The mixture is incubated at 20° C. for 15 min, and an oxidized peptide is purified by preparative HPLC using a BioCAD/SPRINT System from PerSeptive Biosystems (Cambridge, Mass., USA), a Vydac C-18 column (22 mm×250 mm) and a two-solvent system: A, trifluoroacetic acid/water (1:1000, v/v); B, trifluoroacetic acid/acetonitrile/water (1:900:100, v/v). The column is heated to 50° C. in a water bath and peptides (modified and unmodified) are eluted at a flow rate of 15 ml/min with 28-49%, 49-53% and 53-73% gradient steps of solvent B over 12, 9 and 12 min, respectively. Then the content of solvent B is increased to 100% over 3 min, and finally decreased to 28% over 2 min. Peaks are identified by analytical HPLC. Analytical HPLC is performed using a Waters Automated Gradient Controller, a Waters 745B Data Processor and a Thermo Separation Products Spectra 100 UV-visible detector, coupled to a Vydac C-18 column (4.6 mm×250 mm) and heated to 50° C. Peptide is eluted with the same two-solvent system at a flow rate of 1.2 ml/min and 28-64% gradient of B over 33 min. Then the content of B is increased to 100% over 2 min, and finally decreased to 28% over 2 min. The HPLC column eluates are monitored by absorbance at 214 nm. Mass spectra of a purified modified peptide is measured using a Voyager Elite STR mass spectrometer from PerSeptive Biosystems (Cambridge, Mass., USA). Conversion of one methionine residue to methionine sulfoxide results in increasing the molecular weight of the peptide by 16 atomic mass units corresponding to an addition of one extra oxygen atom to the peptide molecule.

### Example 2A: Preparation of Fluorescently Labeled Peptides

Fluorescently labeled peptides can be readily synthesized using the standard methods of peptide labeling that are well known in the art and described, for example, in Cunningham et al. J Biol Chem 2001, 278: 43390-43399; Rojas, et al. Biochim Biophys Acta 2018, 1864:2761-2768; and Shen and Sigalov J Cell Mol Med 2017, 21:2524-2534.

To synthesize rhodamine (rho) B-labeled G-KV21, the labeled peptide can be prepared by solid phase peptide synthesis on p-benzyloxybenzyl alcohol/polystyrene resin using alpha-N-(9-fluorenyl)methoxycarbonyl (alpha-Fmoc) protection chemistry and carbodiimide/N-hydroxybenzotriazole coupling. The side chains are protected as follows: Arg (Pmc), Gln (Trt), Lys (Boc). To couple rho B on this peptide, N-Hydroxysuccinimide (NHS)-Rhodamine is used and linked directly to the N terminus of the peptide on the solid phase support. After coupling, the peptide is cleaved from the solid support with trifluoroacetic acid and phenol (95:5, v/w). The peptide is purified by RP-HPLC on a Silica Gel C18 column using a 20-60% acetonitrile gradient in 0.1% trifluoroacetic acid and dried.

In one embodiment, to prepare fluorescently labeled G-KV21, the peptide was solubilized using 0.1 M phosphate, pH 8, reacted with two-fold molar excess of either DyLight 488 or rhodamine (rho) B N-hydroxysuccinimide (NHS) esters and incubated at 25° C. for 3 hr. The reactions were quenched using ethanolamine. The reaction mixtures were purified using RP-HPLC.

In one embodiment, the fluorescently labeled peptides can be prepared by solid phase peptide synthesis on p-benzyloxybenzyl alcohol/polystyrene resin using alpha-N-(9-fluorenyl)methoxycarbonyl (alpha-Fmoc) protection chemistry and carbodiimide/N-hydroxybenzotriazole coupling. The side chains are protected as follows: Arg (Pmc), Gln (Trt), Lys (Boc). To couple rho B on the peptide, ester derivatives of fluorophores are used and linked directly to the N terminus of the peptide on the solid phase support. After coupling, the peptides are cleaved from the solid support with trifluoroacetic acid and phenol (95:5, v/w). The peptides are purified by RP-HPLC on a Silica Gel C18 column using a 20-60% acetonitrile gradient in 0.1% trifluoroacetic acid and dried.

In one embodiment, fluorescently labeled unprotected unmodified and methionine sulfoxidized peptides can be purchased from specialized companies (i.e., Bachem, Torrance, Calif., USA) with greater than 95% purity as assessed by HPLC. Peptide molecular mass can be checked by matrix-assisted laser desorption ionization mass spectrometry.

### Example 3A: Isolation and Characterization of Native High Density Lipoproteins (HDL)

To isolate and purify native HDL, the standard procedure known in the art can be used (Sigalov et al. J Chromatogr 1991, 537:464-468). Briefly, HDL of density 1.063-1.210 g/ml were isolated from mouse serum by sequential ultracentrifugation in a Beckman Optima LE-80K ultracentrifuge (Berkeley, Calif., U.S.A.) using a fixed-angle 42.2Ti rotor. First, 1 ml mouse serum was mixed with 5 ml of NaCl solution (1.0214 g/ml) to adjust to 1.019 g/ml and centrifuged at 40,000 rpm for 18 h at 18° C. The top 1 ml was removed by aspiration as the VLDL fraction, and the subnatant density was adjusted to 1.063 g/ml by solid KBr and centrifuged similarly for 24 h. The top 1 ml (1.019-1.063 g/ml) was removed by aspiration as the LDL fraction, and the subnatant containing the HDL fraction was adjusted to 1.210 g/ml by solid KBr and centrifuged similarly for 40 h. The HDL fraction isolated by aspiration was extensively dialyzed against phosphate-buffered saline (PBS), pH 7.0 and analyzed in triplicate for cholesterol by an enzymatic colorimetric procedure (diagnostic kit no. 352, Sigma Chemicals, St. Louis, Mo.). Cholesterol values were determined using standard curves obtained by running several concentrations of standards provided with the kit. Protein concentrations were measured using the bicinchoninic acid assay (Pierce, Rockford, Ill., USA) with BSA as a standard.

### Example 4A: Ultracentrifugation of Fluorescently Labeled Peptides

The standard ultracentrifugation procedure known in the art can be used (see for example, Sigalov et al. J Chromatogr 1991, 537:464-468). Briefly, in one embodiment, to prepare delipoproteinized mouse serum, 274 mg solid KBr were dissolved in 200 μl whole mouse serum (final density=1.21 g/ml) and added to 7-by-20 mm ultracentrifugation tubes (final density=1.21 g/ml). Tubes were centrifuged in a 72-position rotor (type 42.2 TI) at 40000 rpm for 16 h at 18° C. 30 μl were collected by aspiration as the lipoprotein fraction and the subnatant was further used as a delipoproteinized mouse serum (density=1.21 g/ml). In one embodiment, 10 μl rho B-labeled GF9, G-TE21, G-HV21 or G-KV21 in PBS, pH 7.4 were mixed with 100 μl whole mouse serum and 130 μL KBr (density=1.37 g/mL) and added to 7-by-20 mm ultracentrifugation tubes (final density=1.21 g/mL). Tubes were centrifuged in a 72-position rotor (type 42.2 TI) at 40000 rpm for 16 h at 18° C. Then, pictures were taken. In one embodiment, 10 μl rho B-labeled GF9, G-TE21, G-HV21 or G-KV21 in PBS, pH 7.4 were mixed with 230 μl delipoproteinized mouse serum (density=1.21 g/ml) and 7 μL KBr (density=1.37 g/mL) and added to 7-by-20 mm ultracentrifugation tubes (final density=1.21 g/mL). Tubes were centrifuged in a 72-position rotor (type 42.2 TI) at 40000 rpm for 16 h at 18° C. Then, pictures were taken.

This example demonstrates that ultracentrifugation can be used to test the ability of the peptides and compounds of the present invention to interact with native lipoproteins. This example further demonstrates that depending on amphipathicity, peptides of the same length may (G-KV21 and G-TE21) or may not (G-TE21) interact with native lipoproteins. This example further demonstrates that TREM-1 inhibitory peptide GF9 does not interact with native lipoproteins. See FIG. 27.

### Example 5A: Preparation and Characterization of Synthetic Lipopeptide Particles

Synthetic lipoprotein/lipopeptide particles (SLP) can be readily reconstituted in vitro from lipids and apolipoproteins. The standard methods of reconstitution and procedures of SLP purification and characterization that are well known in the art and described in Rojas, et al. Biochim Biophys Acta 2018, 1864:2761-2768; Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; and disclosed in Sigalov US 20130045161 and Sigalov US 20110256224 were used to reconstitute SLP as spherical or discoidal particles using from the peptides and compounds of the present invention and lipids. Exemplary use of TREM-1 trifunctional peptide G-KV21 is described below.

### Reagents

1,2-dimyristoyl-sn-glycero-3-phosphocholine (DMPC), 1,2-dimyristoyl-sn-glycero-3-phospho-(1′-rac-glycerol) (DMPG), 1,2-dimyristoyl-sn-glycero-3-phosphoethanolamine-N-(lissamine rhodamine B sulfonyl) (rhodamine-PE, Rho B-PE), 1-palmitoyl-2-oleoyl-sn-glycero-3-phosphocholine (POPC), 1-palmitoyl-2-oleoyl-sn-glycero-3-phospho-(1′-rac-glycerol) (sodium salt) (POPG), 1,2-dimyristoyl-sn-glycero-3-phosphoethanolamine-N-diethylenetriaminepentaacetic acid (gadolinium salt) (14:0 PE-DTPA (Gd)), and egg yolk L-α-phosphatidyl choline (egg-PC) were purchased from Avanti Polar Lipids (Alabaster, Ala.). Sodium cholate, cholesterol, cholesteryl oleate, hydrogen peroxide and other chemicals were purchased from Sigma Chemical Company (St. Louis, Mo.).

Discoidal SLP. Discoidal SLP that contain TREM-1/TRIOPEP G-KV21 (G-KV21-dSLP) were prepared using a general procedure described elsewhere (see e.g., Rojas, et al. Biochim Biophys Acta 2018, 1864:2761-2768; Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; Sigalov US 20130045161, and Sigalov US 20110256224). In one embodiment, the molar ratio was 28:12:1 for DMPC:DMPG:G-KV21. DMPC and DMPG in organic solvents were mixed, dried in a stream of argon, and placed under vacuum for 8 h. In one embodiment, the molar ratio was 65:25:3 for POPC:POPG:G-KV21. POPC and POPG in organic solvents were mixed, dried in a stream of argon, and placed under vacuum for 8 h. To synthesize fluorescently labeled nanoparticles, rhodamine B-PE in chloroform was also added to a lipid mixture. To synthesize Gd-labeled nanoparticles, 14:0 PE-DTPA (Gd) in chloroform was also added to a lipid mixture. Then, lipid films were dispersed in phosphate-buffered saline (PBS), pH 7.4, sonicated for 5 min, and aqueous solution of either oxidized or unmodified G-KV21 was added. Amount of G-KV21 was controllably varied in different preparations. Then, the mixture was incubated for 3 h at 30° C.

Spherical SLP. Spherical SLP that contain TREM-1/TRIOPEP G-KV21 (G-KV21-sSLP) were prepared using a general sodium cholate dialysis procedure described elsewhere (see e.g., Rojas, et al. Biochim Biophys Acta 2018, 1864:2761-2768; Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; Sigalov US 20130045161, and Sigalov US 20110256224). In one embodiment, the molar ratio was 60:3:1:1:103 for egg-PC:cholesterol:cholesteryl oleate:G-KV21:sodium cholate. Egg-PC, cholesterol, and cholesteryl oleate in organic solvents were mixed, dried in a stream of argon, and placed under vacuum for 8 h. In one embodiment, the molar ratio was 125:6:2:3:1:210 for POPC:cholesterol:cholesteryl oleate:G-KV21:sodium cholate. POPC, cholesterol, and cholesteryl oleate in organic solvents were mixed, dried in a stream of argon, and placed under vacuum for 8 h. To synthesize fluorescently labeled nanoparticles, rhodamine B-PE in chloroform was also added to a lipid mixture. To synthesize Gd-labeled nanoparticles, 14:0 PE-DTPA (Gd) in chloroform was also added to a lipid mixture. Then, lipid films were dispersed in Tris-buffered saline-EDTA (TBS-EDTA, pH 7.4), sonicated for 5 min and incubated for 30 min at 30° C. To the dispersed lipids, aqueous solution of either oxidized or unmodified G-KV21 was added. Amount of G-KV21 was controllably varied in different preparations. Then, sodium cholate solution was added and the mixture was incubated at 30° C. for 3 h, followed by extensive dialysis against PBS to remove sodium cholate.

Purification and characterization of discoidal and spherical G-KV21-containing SLP. The obtained G-KV21-SLP particles were purified on a calibrated Superdex 200 HR gel filtration column (GE Healthcare Biosciences, Pittsburgh, Pa.) using the BioCAD 700E Workstation (Applied Biosystems, Carlsbad, Calif.) and characterized by analytical RP-HPLC and non-denaturing gel electrophoresis. Peptide concentrations in the G-KV21-SLP particles were measured as described in Sigalov and Stern. Chem Phys Lipids 2001, 113:133-146. Final peptide compositions were determined in the prepared particles by analytical RP-HPLC essentially as previously described in Sigalov and Stern. Chem Phys Lipids 2001, 113:133-146. Total cholesterol was determined enzymatically using a Boehringer-Mannheim kit and the manufacturer's suggested procedure. Phospholipids were determined by a phosphorus assay. The mean size of the particles was determined using electron microscopy (EM) essentially as described in Sigalov and Stern. Chem Phys Lipids 2001, 113:133-146 and Sigalov. Contrast Media Mol Imaging 2014, 9:372-382. Briefly, the G-KV21-SLP complexes (at a concentration of about 0.3 mg of G-KV21/ml) were extensively dialyzed against 5 mM ammonium bicarbonate, mixed with the same volume of 2% phosphotungstate, pH 7.4, and examined using a FEI Tecnai 12 Spirit BioTwin transmission electron microscope (FEI Company, Hillsboro, Oreg.) at 80 KV accelerating voltage on carbon-coated Formvar grids. Microphotographs were photographed at an instrument magnification of 87000× and 92000×, and mean particle dimensions of 100 particles were determined from each negative.

This example demonstrates that SLP are self-assembled upon binding of the trifunctional peptides and compounds of the invention to lipids This example further demonstrates that depending on method of preparation and composition of lipid mixtures, SLP of discoidal or spherical morphology can be prepared. This example further demonstrates that SLP of discoidal or spherical morphology that contain different imaging probes including, but not limited to, Gd-based contrast agents (GBCA) or rhodamine B fluorescent label can be prepared depending on the imaging probe-conjugated lipids used.

### Example 6A: In Vitro Macrophage Endocytosis of Fluorescent Peptides

In vitro studies of macrophage endocytosis of fluorescently labeled (rho B-labeled) peptides GF9, G-TE21, G-HV21 and G-KV21 in the presence or absence of HDL isolated and purified as described above were performed using the standard methods well known in the art (see e.g. Shen and Sigalov Mol Pharm 2017, 14:4572-4582; Shen and Sigalov J Cell Mol Med 2017, 21:2524-2534; Shen and Sigalov Sci Rep 2016, 6:28672; Shen, et al. PLoS One 2015, 10:e0143453; Sigalov. Contrast Media Mol Imaging 2014, 9:372-382; Sigalov. Int Immunopharmacol 2014, 21:208-219; Sigalov US 20130045161; and Sigalov US 20110256224).

The BALB/c murine macrophage cell line J774A.1 (ATCC TIB-67) were obtained from the American Type Culture Collection (ATCC, Manassas, Va.). The macrophage cells were cultured at 37° C. with 5% CO₂ in Dulbecco's modified Eagle's medium (DMEM) (Cellgro, Mediatech Inc, Manassas, Va.) with 2 mM glutamine, 100 U/ml penicillin, 0.1 mg/ml streptomycin, and 10% fetal bovine serum (FBS) (Cellgro, Mediatech Inc, Manassas, Va.) and grown to approximately 90% of confluence in 6-well tissue culture plates (Corning, Tewksbury, Mass.). Cells were incubated for varied time periods from 4 to 24 h at 37° C. with fluorescently labeled peptides (pre-incubated with 1 mg/ml HDL) at a concentration of 4 μM rhodamine B (rho-B). After incubation, cells were washed twice with PBS and lysed using Promega passive lysis buffer (Promega, Madison, Wis.). The rho B fluorescence was measured in the lysates with a 540 nm excitation and a 590 nm emission filters using the Gemini EM fluorescence microplate reader (Molecular Devices, Sunnyvale, Calif.). The protein concentration in the lysates was determined using the Bradford reagent (Bio-Rad, Richmond, Calif.) and the SpectraMax 190 microplate reader (Molecular Devices, Sunnyvale, Calif.).

In one embodiment, after reaching target confluency, cells were incubated for 1 h in medium with or without fucoidan (400 μg/mL), BLT-1 (10 μM) or cytochalasin D (40 μM), Cells were subsequently incubated for 4 h and 22 h at 37° C. in medium containing 2 μM of rho B-labeled peptides G-KV21 and G-HV21 (as calculated for rho B) that were pre-incubated with 1 mg/ml HDL. After incubation, cells were washed twice with PBS and lysed using Promega passive lysis buffer. The rho B fluorescence and protein concentration were measured in the lysates as described above.

This example demonstrates that pre-incubation with HDL enhances in vitro macrophage endocytosis of G-HV21 and G-KV21 but not GF9 and G-TE21, This example further demonstrates that sulfoxidized methionine residue in TREM-1-related control peptide G-TE21 does not promote macrophage endocytosis neither in the presence or absence of HDL. See FIGS. 34B and 34C. This example further demonstrates that in vitro macrophage endocytosis of TREM-1/TRIOPEP G-KV21 and G-HV21 pre-incubated with HDL is mediated with scavenger receptors (SR) A (SR-A) and BI (SR-B1) with SR-A being the predominant mediator. See FIG. 34A.

### Example 7A: Immunofluorescence Analysis of TREM-1/TRIOPEP G-KV21 in the Cell Membrane

Immunofluorescence analysis of TREM-1/TRIOPEP G-KV21 in the cell membrane was performed using the standard, well-known in the art methods as described in Shen and Sigalov J Cell Mol Med 2017, 21:2524-2534.

BALB/c murine macrophage J774A.1 cells were grown at 37° C. in six-well tissue culture plates containing glass coverslips. After reaching target confluency of approximately 50%, cells were incubated for 6 h at 37° C. with Dylight 488-labeled G-KV21 that was pre-incubated with HDL. TREM-1 staining was performed using an Alexa 647-labeled rat anti-mouse TREM-1 antibody (Bio-Rad, Hercules, Calif.). ProLong Gold Antifade DAPI (4′,6′-diamidino-2-phenylindole) mounting medium was used to mount coverslips, and the slides were photographed using an Olympus BX60 fluorescence microscope. Confocal imaging was performed with a Leica TCS SP5 II laser scanning confocal microscope.

This example demonstrates that upon endocytosis by macrophages, TREM-1/TRIOPEP G-KV21 is released by native lipoproteins, self-inserts into the cell membrane and colocalizes with TREM-1. See FIG. 32.

### Example 8A: In Vitro Cytokine Release

In vitro studies of cytokine release by lipopolysaccharide (LPS)-stimulated macrophages in the presence of GF9, G-TE21, G-HV21 and G-KV21 either pre-incubated or not with HDL were performed using the standard, well-known in the art methods as described in Sigalov. Int Immunopharmacol 2014, 21:208-219.

The BALB/c murine macrophage cell line J774A.1 (ATCC TIB-67) were obtained from the American Type Culture Collection (ATCC, Manassas, Va.). Macrophages were cultured in 48-well plates (Corning, Cambridge, Mass.) for 24 h at 37° C. in the presence of LPS (1 μg/ml, Escherichia coli 055:B5, Sigma) in combination with 10 ng/ml peptides either pre-incubated or not with HDL. Cell-free supernatants were harvested and stored at −20° C. for later cytokine quantification. TNF-alpha, IL-6, and IL-1beta were assayed using commercial ELISA kits (Pierce Biotechnology, Thermo Scientific, Rockford, Ill.) according to the recommendations of the manufacturer. Results were represented as the mean±S.D. of three independent experiments. Statistical significances in in vitro macrophage uptake assay were determined by two-tailed Student's t test.

This example demonstrates that after pre-incubation with HDL, G-HV21 and G-KV21 but not GF9 or G-TE21 inhibit production of cytokines by LPS-stimulated macrophages. This example further demonstrates that after pre-incubation with HHDL, G-TE21 does not affect on cytokine release by LPS-stimulated macrophages. See FIG. 33.

### Example 9A: Mouse Model of LPS-Induced Endotoxemia and In Vivo Survival and Cytokine Release Studies

Animal survival studies and studies of in vivo cytokine release were performed in a mouse model of LPS-induced septic shock using the standard, well known in the art methods as described in Sigalov. Int Immunopharmacol 2014, 21:208-219.

Naïve, female C57BL/6 mice at 8 to 10 weeks of age (18 to 21 g) from the Jackson Laboratory were randomly grouped (10 mice per group) and i.p. injected with vehicle or the indicated doses of dexamethasone (DEX), GF9, G-TE21, G-HV21 and G-KV21. One hour later, mice received i.p. injection of 30 mg/kg LPS from E. coli 055:B5 (Sigma). In some experiments, all formulations were i.p. administered 1 and 3 h after LPS injection. The viability of mice was examined hourly. Body weights were measured daily. In all of the animal experiments, blood samples were collected via a sub-mandibular (cheek) bleed at 90 min after administration of LPS. Statistical analysis of survival curves was performed by the Kaplan-Meier test. Comparisons were made using two-tailed Student's t test. The production of cytokines in serum was measured by a standard sandwich cytokine ELISA procedure using TNF-alpha, IL-1beta and IL-6 ELISA kits (Pierce Biotechnology, Thermo Scientific, Rockford, Ill.) according to the instructions of the manufacturer. Statistical significances in cytokine analysis ELISA data were determined by two-tailed Student's t test.

This example demonstrates that at this dose, G-HV21 and G-KV21 but not GF9 and G-TE21 inhibit LPS-stimulated cytokine production in vivo. This example further demonstrates that at this dose, G-HV21 and G-KV21 but not GF9 and G-TE21 protect mice from LPS-induced septic shock and prolongs survival of septic mice. See FIGS. 35 and 36A-B.

### Example 10A: Lung Cancer Tumor Xenografts in Nude Mice and In Vivo Tumor Growth Studies

Animal efficacy studies were performed in human xenograft mouse models of NSCLC using female 6-8 week old NU/J mice from the Jackson Laboratory (Bar Harbor, Me.) using the standard, well-known in the art methods as described in Sigalov. Int Immunopharmacol 2014, 21:208-219 and disclosed in Wu, et al. U.S. Pat. No. 8,415,453 and Sigalov U.S. Pat. No. 8,513,185.

Animal efficacy studies were performed using female 6-8 week old NU/J mice from the Jackson Laboratory (Bar Harbor, Me.). Animals were handled as specified in the USDA Animal Welfare Act (9 CFR, Parts 1, 2, and 3) and as described in the Guide for the Care and Use of Laboratory Animals from the National Research Council. Human lung carcinoma cell lines H292 and A549 were obtained from ATCC. Tumor cells in culture were harvested and resuspended in a 1:1 ratio of RPMI 1640 and Matrigel (BD Biosciences, San Jose, Calif.). NSCLC xenografts were established by injecting subcutaneously into the right flanks 5×10⁶ viable cells per mouse. Tumor volumes were calculated with caliper measurements using the formula V=π/6 (length×width×width). When tumor volumes reached an average of 200 mm³, tumor-bearing animals were randomized into groups of 10, and dosing of PBS (vehicle), paclitaxel (PTX) or peptides G-TE21, G-HV21 and G-KV21 was initiated. All tested formulations were intraperitoneally (i.p.) injected at indicated doses and administration schedule. Clinical observations, body weights and tumor volume measurements were made 3 times weekly. Tumor volumes were analyzed using repeated measures ANOVA followed by Bonferroni test. Data points were represented as mean tumor volume±SEM. Antitumor effects were expressed as the percentage of T/C (treated versus control), dividing the tumor volumes from treatment groups with the control groups and multiplied by 100. According to the National Cancer Institute (NCI) standards (see e.g., Johnson, et al. Br J Cancer 2001, 84:1424-1431), a % T/C≤42 is indicative of antitumor activity. At the end of the experiment, the animals were sacrificed and the tumors were excised and weighed.

This example demonstrates that G-HV21 and G-KV21 but not G-TE21 inhibit tumor growth in two human NSCLC xenograft mouse models. See FIGS. 37 and 38.

### Example 11A: Pancreatic Cancer Tumor Xenografts in Nude Mice and In Vivo Tumor Growth and Survival Studies

In order to demonstrate that the TREM-1-related TRIOPEP peptides are effective in inhibiting TREM-1-mediated cell activation and reducing pancreatic tumor (PC) growth, animal efficacy studies were performed in human xenograft mouse models of PC using 5-6 week old female athymic nude-Foxn1^{nu} mice obtained from Envigo (formerly Harlan, Inc.) using the standard, well known in the art methods as described in Shen and Sigalov. Mol Pharm 2017, 14:4572-4582.

### Animal Studies

Mice were implanted subcutaneously into the right flank with 5×10⁶ AsPC-1, BxPC-3, CAPAN-1 or PANC-1 cells in equal parts of serum-free growth medium and Matrigel. Mice were monitored daily and tumor measurements were taken along the length and width using Vernier calipers twice weekly until sacrifice. Tumor volumes were calculated using a modified ellipsoidal formula: (Length×Width²)/2. In one embodiment, when the AsPC-1, BxPC-3 and CAPAN-1 tumors reached a calculated volume of approximately 150-200 mm³, mice were sorted into treatment groups and PBS (vehicle), PTX, G-HV21, G-KV21 or G-TE21 were i.p. injected once daily for 5 days per week at indicated doses. Treatment persisted for 31 days for AsPC-1-containing mice and 29 days for mice containing established BxPC-3 and Capan-1 xenograft tumors. In one embodiment, when the PANC-1 tumors reached a calculated volume of approximately 150-200 mm³, mice were sorted into treatment groups and i.p. dosing with PBS (vehicle), chemotherapy (100 mg/kg gemcitabine+10 mg/kg Abraxane, “GEM+ABX”) either with (G-KV21+GEM+ABX) or without (GEM+ABX) 5 mg/kg G-KV21 was started. Treatment with GEM+ABX applied once daily at days 1, 4, 8, 11 and 15. Treatment with PBS or G-KV21 persisted for 30 days once daily for 5 days per week. Mice were humanely sacrificed when individual tumors exceeded 1500 (BxPC-3) or 2000 (PANC-1) mm³.

### Immunohistochemistry

Mice containing BxPC-3 tumors were humanely euthanized for necropsy at the end of the study. Excised tumors were fixed using 10% neutral buffered formalin for 1-2 days, processed for paraffin embedding, and sectioned at 4 m. Antigen retrieval for F4/80 was achieved using Proteinase K (Dako North America). Sections were blocked for peroxidase and alkaline phosphatase activity using Dual Endogenous Enzyme Block (Dako North America). Sections were then incubated with Protein Block (Dako North America) followed by primary antibody F4/80 (1:2000, AbD Serotec) diluted using 1% bovine serum albumin in Tris-buffered saline. Afterward, sections were incubated using EnVision+ secondary antibodies (Dako North America), followed by 3,3′-diaminobenzidine in chromogen solution (Dako North America) and counterstained using hematoxylin (Dako North America). Quantitative analysis of intratumoral F4/80 staining was determined using Visiopharm software.

### Cytokine Detection

Blood was collected on study days 1 and 8 and processed into serum. Serum cytokines were analyzed by Quantibody Mouse Cytokine Array Q1 kits (RayBiotech) according to the manufacturer's instructions.

### Statistical Analysis

Results are expressed as the mean±SEM. Statistical differences were analyzed using analysis of variance with Bonferroni adjustment unless otherwise noted. The Kaplan-Meier method was used to estimate survival as a function of time, and survival differences were analyzed by the log-rank test. p values less than 0.05 were considered significant.

This example demonstrates that G-HV21 and G-KV21 but not G-TE21 inhibit tumor growth in three human PC xenograft mouse models. This example further demonstrates that TREM-1 blockade using G-HV21 and G-KV21 reduces the macrophage infiltration into the tumor. This example further demonstrates that treatment with G-KV21 does not affect the macrophage infiltration into the tumor. This example further demonstrates that being applied with chemotherapy, TREM-1/TRIOPEP sensitizes the PANC-1 tumor to chemotherapy and significantly prolongs survival. See FIGS. 36A-B (shown for BxPC-3) and 40A.

### Example 12A: Mouse Tolerability Studies

Mouse tolerability studies were performed in healthy C57BL/6 mice using the standard, well-known in the art methods as described in Sigalov. Int Immunopharmacol 2014, 21:208-219.

Naïve, female C57BL/6 mice at 8 to 10 weeks of age (18 to 21 g) from the Jackson Laboratory were used. Animals were randomly grouped (5 mice per group) and i.p. injected with 400 mg/kg G-HV21, G-KV21 or G-TE21. Clinical observations and body weights were made twice daily.

This example demonstrates that G-HV21, G-KV21 and G-TE21 all are non-toxic and well tolerated in healthy mice at doses of up to at least 400 mg/kg. See FIG. 41.

### Example 13A: Haemodynamic Studies in Septic Rats

The role of TREM-1-related trifunctional peptides in further models of septic shock, is investigated by performing LPS- and cecal ligation and puncture (CLP)-induced endotoxinemia experiments in rats. The experiments can be conducted analogously to those described in Gibot, et al. Infect Immun 2006, 74:2823-2830 and disclosed in Faure, et al. U.S. Pat. No. 8,013,116; Faure, et al. U.S. Pat. No. 9,273,111; and Sigalov U.S. Pat. No. 8,513,185.

### LPS-Induced Endotoxinemia

Animals are randomly grouped (n=10-20) and treated with Escherichia coli LPS (0111:B4, Sigma-Aldrich, Lyon, France) i.p. in combination with G-HV21, G-KV21 or G-TE21 at various concentrations.

### CLP Polymicrobial Sepsis Model

Rats (n=6-10 per group) are anesthetized by i.p. administration of ketamine (150 mg/kg). The caecum is exposed through a 3.0-cm abdominal midline incision and subjected to a ligation of the distal half followed by two punctures with a G21 needle. A small amount of stool is expelled from the punctures to ensure potency. The caecum is replaced into the peritoneal cavity and the abdominal incision closed in two layers. After surgery, all rats are injected s.c. with 50 mL/kg of normal saline solution for fluid resuscitation. G-HV21, G-KV21 or G-TE21 are then administered at various concentrations.

### Haemodynamic Measurements in Rats

Immediately after LPS administration as well as 16 hours after CLP, arterial BP (systolic, diastolic, and mean), heart rate, abdominal aortic blood flow, and mesenteric blood flow are recorded. Briefly, the left carotid artery and the left jugular vein are cannulated with PE-50 tubing. Arterial BP is continuously monitored by a pressure transducer and an amplifier-recorder system (IOX EMKA Technologies, Paris, France). Perivascular probes (Transonic Systems, Ithaca, N.Y.) are wrapped up the upper abdominal aorta and mesenteric artery, allowed to monitor their respective flows by means of a flowmeter (Transonic Systems). After the last measurement (4^{th} hour after LPS and 24^{th} hour after CLP), animals are sacrificed by an overdose of sodium thiopental i.v. (intravenously).

### Biological Measurements

Blood is sequentially withdrawn from the left carotid artery. Arterial lactate concentrations and blood gases analyses are performed on an automatic blood gas analyser (ABL 735, Radiometer, Copenhagen, Denmark). Concentrations of TNF-alpha and IL-1beta in the plasma are determined by an ELISA test (Biosource, Nivelles, Belgium) according to the recommendations of the manufacturer. Plasmatic concentrations of nitrates/nitrites are measured using the Griess reaction (R&D Systems, Abingdon, UK).

### Statistical Analyses

Between-group comparisons are performed using Student's t tests. All statistical analyses are completed with Statview software (Abacus Concepts, Calif.).

### Example 14A: Attenuation of Intestinal Inflammation in Animal Models of Colitis

In order to demonstrate that the TREM-1-related TRIOPEP formulations are effective in inhibiting TREM-1-mediated cell activation in animal models of colitis, the experiments can be conducted analogously to those described in Schenk, et al. J Clin Invest 2007, 117:3097-3106 and disclosed in Faure, et al. U.S. Pat. No. 8,013,116; Faure, et al. U.S. Pat. No. 9,273,111 and Sigalov U.S. Pat. No. 8,513,185.

### Mice

C57BL/6 mice, purchased from Harlan, and C57BL/6 RAG2−/− mice, bred in a specific pathogen-free (SPF) animal facility, are used at 8-12 weeks of age. All experimental mice are kept in micro-isolator cages in laminar flows under SPF conditions.

### Mouse Models of Colitis

For experiments involving the adoptive T cell transfer model, colitis is induced in C57BL/6 RAG2−/− mice by adoptive transfer of sorted CD4+CD45RBhigh T cells. Briefly, CD4+ T cells are isolated from splenocytes from C57BL/6 mice, and after osmotic lysis of erythrocytes, CD4+ T cells are enriched by a negative MACS procedure for CD8alpha and B220 (purified, biotinylated, hybridoma supernatant) using avidin-labeled magnetic beads (Miltenyi Biotec). Subsequently, the CD4+ T cell-enriched fraction is stained and FACS sorted for CD4+(RM4-5; BD Biosciences—Pharmingen), CD45RBhi (16A; BD Biosciences—Pharmingen), and CD25− (PC61; eBioscience) naive T cells. Each C57BL/6 RAG2−/− mouse is injected i.p. with 1×105 syngeneic CD4+CD45RBhighCD25− T cells. Colitic mice are sacrificed and analyzed on day 14 after adoptive transfer.

For experiments involving the dextran sodium sulfate (DSS) colitis model, C57BL/6 mice are given autoclaved tap water containing 3% DSS (DSS salt, reagent grade, mol wt: 36-50 kDa; MP Biomedicals) ad libitum over a 5-day period. The consumption of 3% DSS is measured. DSS is replaced thereafter by normal drinking water for another 4 days. Mice are euthanized and analyzed at the end of the 9-day experimental period.

### Treatment

Upon colitis induction, either starting on day 0 or after onset of colitis on day 3, mice are treated with G-HV21, G-KV21 or G-TE21 i.p. injected at various concentrations in 200 ul saline.

### Colitis Scoring

At the end of the experiments, the colon length is measured from the end of the cecum to the anus. Fecal samples are tested for occult blood using hemo FEC (Roche) tests (score 0, negative test; 1, positive test and no rectal bleeding; 2, positive test together with visible rectal bleeding). The colon is divided into 2 parts. From each mouse, identical segments from the distal and proximal colon are taken for protein and RNA isolation and histology, and frozen tissue blocks are prepared for subsequent analysis. Histological scoring of paraffin-embedded H&E-stained colonic sections is performed in a blinded fashion independently by 2 pathologists. To assess the histopathological alterations in the distal colon, a scoring system is established using the following parameters: (a) mucin depletion/loss of goblet cells (score from 0 to 3); (b) crypt abscesses (score from 0 to 3); (c) epithelial erosion (score from 0 to 1); (d) hyperemia (score from 0 to 2); (e) cellular infiltration (score from 0 to 3); and (f) thickness of colonic mucosa (score from 1 to 3). These individual histology scores are added to obtain the final histopathology score for each colon (0, no alterations; 15, most severe signs of colitis).

### RNA Isolation and RT-PCR

RNA is isolated from intestinal tissue samples preserved in RNAlater (QIAGEN), using the RNAeasy Mini Kit (QIAGEN). RT-PCR is performed with 400 ng RNA each, using the TaqMan Gold RT-PCR Kit (Applied Biosystems). Primers are designed as follows: mouse TREM-1, forward 5′-GAGCTTGAAGGATGAGGAAGGC-3′ and reverse 5′-CAGAGTCTGTCACTTGAAGGTCAGTC-3′; mouse TNF, forward 5′-GTAGCCCACGTCGTAGCAAA-3′ and reverse 5′-ACGGCAGAGAGGAGGTTGAC-3′; mouse beta-actin, forward 5′-TGGAATCCTGTGGCATCCATGAAAC-3′ and reverse 5′-TAAAACGCAGCTCAGTAACAGTCCG-3′; human TREM-1, forward 5′-CTTGGTGGTGACCAAGGGTTTTTC-3′ and reverse 5′-ACACCGGAACCCTGATGATATCTGTC-3′; human TNF, forward 5′-GCCCATGTTGTAGCAAACCC-3′ and reverse 5′-TAGTCGGGCCGATTGATCTC-3′; human GAPDH, forward 5′-TTCACCACCATGGAGAAGGC-3′ and reverse 5′-GGCATGGACTGTGGTCATGA-3′. PCR products are semiquantitatively analyzed on agarose gels.

Human TREM-1 and mouse TREM-1 and TNF expression is also assessed by real-time PCR using the TREM-1 QuantiTect primer assay system and QuantiTect SYBR green PCR Kit (both from QIAGEN). GAPDH is used to normalize TREM-1 and TNF expression levels. DNA is amplified on a 7500 Real-Time PCR system (Applied Biosystems), and the increase in gene expression is calculated using Sequence Detection System software (Applied Biosystems).

### Western Blot Analysis

Protein samples are separated on a denaturing 12% acrylamide gel, followed by transfer to nitrocellulose filter and probing with the primary antibody. Anti-TREM-1 (polyclonal goat IgG, 0.1 ug/ml; R&D Systems) or anti-tubulin (clone B-5-1-2, 1:5,000; Sigma-Aldrich) is used as primary reagent. As secondary antibodies, HRP-labeled donkey anti-goat Ig (1:2,000; The Binding Site) and goat anti-mouse Ig (1:4,000; Sigma-Aldrich) are used. Binding is detected by chemiluminescence using a Super Signal West Pico Kit (Pierce).

### Statistics

The unpaired 2-tailed Student t test is used to compare groups; P values less than 0.05 are considered significant.

### Example 15A: Autophage Activity and Colitis in Mice

In order to further demonstrate that the TREM-1-related TRIOPEP formulations are effective in inhibiting TREM-1-mediated cell activation in animal models of colitis, the experiments can be conducted analogously to those described in Kokten, et al. J Crohns Colitis 2018, 12:230-244 and disclosed in Faure, et al. U.S. Pat. No. 8,013,116; and Faure, et al. U.S. Pat. No. 9,273,111.

### Animals

In vivo experiments are performed as recommended by the US National Committee on Ethics Reflection Experiment [described in the Guide for Care and Use of Laboratory Animals, NIH, MD, 1985]. The experiments are performed on 25 adult male C57BL/6 mice [Janvier Labs, Le Genest-Saint-Isle, France] and 10 adult male Trem-1 knock-out [TREM-1 KO] C57BL/6 mice [INSERM U1116, Inotrem Laboratory, Nancy, France], all aged between 7 and 9 weeks. The animals are housed at 22-23° C., with a 12 h/12 h light/dark cycle, and ad libitum access to food and water.

### Induction of Colitis, Treatment and Assessment of Disease Activity Index

Colitis is induced by administration of 3% dextran sulfate sodium [DSS, molecular weight 36,000-50,000, MP Biomedical, Strasbourg, France] dissolved in water for 5 days. DSS is replaced thereafter by normal drinking water for another 5 days. Either G-HV21, G-KV21, G-TE21 or the vehicle alone, used as control, are i.p. administered 2 days before colitis induction and then once daily until the last day of DSS administration, at different concentrations in 200 L of saline. This dose is chosen after having performed dose-response experiments. Bodyweight, physical condition, stool consistency, water/food consumption and the presence of gross and occult blood in excreta and at the anus are determined daily. The DAI is also calculated daily by scoring bodyweight loss, stool consistency and blood in the stool on a 0 to 4 scale. 41 The overall index corresponds to the weight loss, stool consistency and rectal bleeding scores divided by three, and thus ranges from 0 to 4.

### Collection of Colon Tissue and Fecal Samples

Ten days after the initiation of colitis with DSS, the mice are sacrificed by decapitation. The colon is quickly removed, opened along its length and gently washed in PBS [2.7 mmol/L KCl, 140 mmol/L NaCl, 6.8 mmol/L Na2HPO4.2H2O, 1.5 mmol/L KH2PO4, pH 7.4]. For histological assessment samples are fixed overnight at 4° C. in 4% paraformaldehyde solution and embedded in paraffin. For protein extractions samples are frozen in liquid nitrogen [−196° C.] and stored at −80° C. For the gut microbiota analysis, whole fecal pellets are collected daily in sterile tubes and immediately frozen at −80° C. until analysis.

### Histological Assessment and Scoring

Colitis is histologically assessed on 5 m sections stained with hematoxylin-eosin-saffron [HES] stain. The histological colitis score is calculated blindly by an expert pathologist.

### Endoscopic Assessment and Scoring

Endoscopy is performed on the last day of the study, just before the mice are sacrificed. Prior to the endoscopic procedure, mice are anaesthetized by isoflurane inhalation. The distal colon [3 cm] and the rectum are examined using a rigid Storz Hopkins II miniendoscope [length: 30 cm; diameter: 2 mm; Storz, Tuttlingen, Germany] coupled to a basic Coloview system [with a xenon 175 light source and an Endovision SLB Telecam; Storz]. Air is insufflated via a 9-French gauge over-tube and a custom, low-pressure pump with manual flow regulation [Rena Air 200; Rena, Meythet, France]. All images are displayed on a computer monitor and recorded with video capture software [Studio Movie Board Plus from Pinnacle, Menlo Park, Calif.]. The endoscopy score is calculated from three subscores: the vascular pattern [scored from 1 to 3], bleeding [scored from 1 to 4] and erosions/ulcers [scored from 1 to 4].

### Western Blot Analysis

Total protein is extracted from the frozen colon samples by lysing homogenized tissue in a radioimmunoprecipitation assay [RIPA] buffer [0.5% sodium deoxycholate, 0.1% sodium dodecyl sulfate [SDS] and 1% NP-40] supplemented with protease inhibitors [Roche Diagnostics, Mannheim, Germany]. Protein is then quantified using the bicinchoninic acid assay method. For each mouse, a total of 20 μg of protein is transferred to a 0.45 m polyvinylidene fluoride [PVDF] or 0.45 m nitrocellulose membrane following electrophoretic separation on a denaturing acrylamide gel. The membrane is blocked with 5% w/v non-fat powdered milk or 5% w/v bovine serum albumin [BSA] diluted in Tris-buffered saline with 0.1% v/v Tween® 20 [TBST] for 1 h at room temperature. The PVDF or nitrocellulose membranes are then incubated overnight at 4° C. with various primary antibodies diluted in either 5% w/v nonfat powdered milk or 5% w/v BSA, TBST. After washing in TBST, the appropriate HRP-conjugated secondary antibody is added and the membrane is incubated for 1 h at room temperature. After further washing in TBST, the proteins are detected using an ECL or ECL PLUS kit [Amersham, Velizy-Villacoublay, France]. Glyceraldehyde 3-phosphate dehydrogenase [GAPDH] is used as an internal reference control.

Enzyme-Linked Immunosorbent Assay [ELISA] for Analysis of Soluble TREM-1 [sTREM-1]

At the time of animal sacrifice, whole blood from each mouse is collected into heparinized tubes. These tubes are centrifuged at 3,000 g for 10 min at 4° C. to collect the supernatants, which are stored at −80° C. until use. Plasma concentration of sTREM-1 is determined by a sandwich ELISA technique using the Quantikine kit assay [RnD Systems, Minneapolis, Minn., USA] according to the manufacturers' instructions. Briefly, samples are incubated with a monoclonal antibody specific for TREM-1 pre-coated onto the wells of a microplate. Following a wash, to eliminate the unbound substances, an enzyme-linked polyclonal antibody specific for TREM-1 is added to the wells. After washing away the unbound conjugate, a substrate solution is added to the wells. Color development is stopped and optical density of each well is determined within 30 min using a microplate reader [Sunrise, Tecan, Mannedorf, Switzerland] set to 450 nm, with a wavelength correction set to 540 nm. All measurements are performed in duplicate and the sTREM-1 concentration is expressed in pg/ml.

### Reverse Transcription-Quantitative Polymerase Chain Reaction

Total RNA is purified from the frozen colon samples with the RNeasy Lipid Tissue kit following the recommendation of Qiagen [Courtaboeuf, France], which includes treatment with DNase. To check for possible DNA contamination of the RNA samples, reactions are also performed in the absence of Omniscript RT enzyme [Qiagen]. Reverse transcription is performed using PrimeScript™ RT Master Mix [TAKARA Bio, USA] according to the manufacturer's recommendations with 200 ng of RNA in a 10 μL reaction volume. PCR is then carried out from 2 μL of cDNA with SYBR® Premix Ex Taq™ [Tli RNaseH Plus] [TAKARA Bio, USA] according to the manufacturer's recommendations in a 20 μL reaction volume, with reverse and forward primers at a concentration of 0.2 μM. Specific amplifications are performed using the following primers: TREM-1, forward 5′-CTGTGCGTGTTCTTTGTC-3′ and reverse 5′-CTTCCCGTCTGGTAGTCT-3′. Quantification is performed with RNA polymerase II [Pol II] as an internal standard with the following primers: forward 5′-AGCAAGCGGTTCCAGAGAAG-3′ and reverse 5′-TCCCGAACACTGACATATCTCA-3′. Temperature cycling for TREM-1 is 30 s at 95° C. followed by 40 cycles consisting of 95° C. for 5 s and 59° C. for 30 s. Temperature cycling for RNA polymerase II is 30 s at 95° C. followed by 40 cycles consisting of 95° C. for 5 s and 60° C. for 30 s. Results are expressed as arbitrary units by calculating the ratio of crossing points of amplification curves of TREM-1 and internal standard by using the δδCt method.

### Microbiota Analysis

For the pharmacologically [with TREM-1/TRIOPEP treatment] inhibition of TREM-1, total DNA is extracted from three pooled fecal pellets from each group of mice [day 0 to day 10; n=33 samples]. For microbiota analysis by MiSeq sequencing, the V3-V4 region [519F-785R] of the 16S rRNA gene is amplified with the primer pair S-DBact-0341-b-S-17/S-D-Bact-0785-a-A-21.45 The following quality filters are applied: minimum length=300 base pairs [bp], maximum length=600 bp and minimum quality threshold=20. This filtering yields an average [range] of 25600 reads/samples [14,553-35,490] for further analysis. High-quality reads are pooled, checked for chimeras [using uchime46], and grouped into operational taxonomic units [OTUs][based on a 97% similarity threshold] using USEARCH 8.0.47 Singletons and OTUs representing less than 0.02% of the total number of reads are removed, and the phylogenetic affiliation of each OTU is assessed with Ribosomal Database Project's taxonomy48 from the phylum level to the species level. The mean [range] number of detected OTUs per sample is 324 [170-404]. In the experiments involving Trem-1 KO mice, similar methods are applied but total DNA is extracted from individual fecal pellets of each mouse from the four groups of animals at baseline [before DSS treatment] and at day 10 [after DSS treatment] [n=37 samples]. Following MiSeq sequencing of the V3-V4 region of the 16S rRNA gene, yielding 2,143,457 raw reads, quality filtering is applied [minimum length=200 bp, maximum length=600 bp and minimum quality threshold=20] and an average [range] of 11,560 reads/samples [7,560-18,495] is kept for further analysis. The mean [range] number of detected OTUs per sample is 599 [131-798].

### Statistical Analysis

A two-tailed Student t test is used to compare two groups and a one-way analysis of variance [ANOVA] is used to compare three or more groups. Bonferroni or Tamhane post hoc tests are applied, depending on the homogeneity of the variance. The threshold for statistical significance is set to p<0.05. The statistical language R is used for data visualization and to perform abundance-based principal component analysis [PCA] and interclass PCA associated with Monte-Carlo rank testing on the bacterial genera.

### Example 16A: Modulation of the TREM-1 Pathway During Severe Hemorrhagic Shock in Rats

In order to demonstrate that the TREM-1-related TRIOPEP formulations are effective in inhibiting TREM-1-mediated cell activation and preventing organ dysfunction and improving survival in rats during severe hemorrhagic shock, the experiments can be conducted analogously to those described in Gibot, et al. Shock 2009, 32:633-637 and disclosed in Faure, et al. U.S. Pat. No. 8,013,116; Faure, et al. U.S. Pat. No. 9,273,111, and Sigalov. U.S. Pat. No. 8,513,185.

### Animals

Adult male Wistar rats (250-300 g) are purchased from Charles River Laboratories (Wilmington, Mass., USA). After 1 week of acclimatization, rats are fasted 12 h before the experiments and are allowed free access to water. All the studies described in the succeeding sentences comply with the regulations concerning animal use and care published by the National Institutes of Health.

### Hemorrhagic Shock Model

Hemorrhagic shock is induced by bleeding from a heparinized (10 UI/mL) carotid artery catheter. Briefly, the rats are anesthetized (50 mg/kg pentobarbital sodium, i.p.) and kept on a temperature-controlled surgical board (37° C.). A tracheostomy is performed, and the animals are ventilated supine (tidal volume, 7-8 mL/kg; rodent ventilator no. 683; Harvard Apparatus, Holliston, Mass.) with a fraction of inspired oxygen of 0.3 and a respiratory rate of 60 breaths per minute. Anesthesia and respiratory support are maintained during the whole experiment. The left carotid artery and the left jugular vein are cannulated with PE-50 tubing. Arterial blood pressure is continuously monitored by a pressure transducer and an amplifier-recorder system (IOX EMKA Technologies, Paris, France). After a 30-min stabilization period, blood is drawn in 10 to 15 min via the carotid artery catheter until MAP reached 40 mmHg. Blood is kept at 37° C., and MAP is maintained between 35 and 40 mm Hg during 60 min. Rats are then allocated randomly (n=10-12 per group) to receive 0.1 mL of either saline (isotonic sodium chloride solution), G-HV21, G-KV21 or G-TE21 at various concentrations in 0.1 mL of saline solution over 1 min via the jugular vein (H0). Shed blood and ringer lactate (volume=3× shed volume) are then infused via the jugular vein in 60 min, and rats are observed for a 4-h period before being killed by pentobarbital sodium overdose. Killing occurs earlier if MAP decreased to less than 35 mm Hg.

### Arterial Blood Gas, Lactate, and Cytokines

Arterial blood gas and lactate concentrations are determined hourly on an automatic blood gas analyzer (ABL 735; Radiometer, Copenhagen, Denmark). Concentrations of TNF-alpha and IL-6 and sTREM-1 in the plasma are determined in triplicate by enzyme-linked immunosorbent assay (Biosources, Nivelles, Belgium; RnD Systems, Lille, France).

### Bacterial Translocation

Rats are killed under anesthesia, and mesenteric lymph node (MLN) complex, spleen, and blood are aseptically removed 4 h after the beginning of reperfusion (or earlier if MAP decreased <35 mm Hg). Homogenates of MLN and spleen and serial blood dilutions are plated and incubated overnight at 37° C. on Columbia blood agar plates (in carbon dioxide and anaerobically) and Macconkey agar (in air). Visible colonies are then counted.

### Pulmonary Integrity

Additional groups of rats (n=4) are subjected to the same procedure but are also infused via the tail vein with fluorescein isothiocyanate (FITC)-albumin (5 mg/kg in 0.3 mL of phosphate-buffered saline) 2 h after the beginning of reperfusion. Rats in these groups are killed 2 h later with an overdose of sodium pentobarbital (200 mg/kg). Immediately thereafter, the lungs are lavaged three times with 1 mL of phosphate-buffered saline, and blood is collected by cardiac puncture. The bronchoalveolar lavage fluid (BALF) is pooled, and plasma is collected. Fluorescein isothiocyanate-albumin concentrations in BALF and plasma are determined fluorometrically (excitation, 494 nm; emission, 520 nm). The BALF-plasma fluorescence ratio is calculated and used as a measure of damage to pulmonary alveolar endothelial/epithelial integrity as previously described (Yang et al. Crit Care Med 2004; 32:1453-9).

### Statistical Analysis

Data are analyzed using ANOVA or ANOVA for repeated measures when appropriate, followed by Newman-Keuls post hoc test. Survival curves are compared using the log-rank test. A two-tailed value of P less than 0.05 is deemed significant. All analyses are performed with GraphPad Prism software (GraphPad, San Diego, Calif.).

### Example 17A: Pharmacological Inhibition of TREM-1 in Experimental Atherosclerosis

In order to further demonstrate that the TREM-1-related TRIOPEP formulations are effective in inhibiting TREM-1-mediated cell activation in animal models of atherosclerosis, the experiments can be conducted analogously to those described in Joffre, et al. J Am Coll Cardiol 2016, 68:2776-2793 and disclosed in Faure, et al. U.S. Pat. No. 8,013,116 and Faure, et al. U.S. Pat. No. 9,273,111.

### Animals

Trem-1^{-/-} mice (null for the Trem-1 gene) are generated (GenOway, Lyon, France) and backcrossed for more than 10 generations into a C57BL/6J background. Ten-week-old male C57BL/6J Ldlr^{-/-} mice are subjected to medullar aplasia by lethal total body irradiation (9.5 Gy). The mice are repopulated with an intravenous injection of bone marrow cells isolated from femurs and tibias of sex-matched C57BL/6J Trem-1^{-/-} mice or Trem-1^{+/+} littermates. After 4 weeks of recovery, mice are fed a proatherogenic diet containing 15% fat, 1.25% cholesterol, and 0% cholate for 4, 8, or 14 weeks. Eight-week old male ApoE^{-/-} mice are blindly randomized and treated daily by i.p. injection of G-HV21, G-KV21 or G-TE21 at various concentrations during 4 weeks and were put on either a chow or a high-fat diet (15% fat, 1.25% cholesterol).

### Extent and Compositions of Atherosclerotic Lesions

Plasma cholesterol is measured using a commercial cholesterol kit. The basal half of the ventricles and ascending aorta are perfusion-fixed in situ with 4% paraformaldehyde. Afterward, they are removed, transferred to a phosphate-buffered saline (PBS)-30% sucrose solution, embedded in frozen optimal cutting temperature compound and stored at [symbol] −70° C. Serial 10-μm sections of the aortic sinus with valves (80 per mouse) are cut on a cryostat. One of every 5 sections is kept for plaque size quantification after Oil Red O (Sigma-Aldrich, St. Louis, Mo.) staining. Thus, 16 sections, spanning an 800-μm length of the aortic root, are used to determine mean lesion area for each mouse. Oil Red O-positive lipid contents are quantified by a blinded operator using HistoLab software (Microvisions Instruments, Paris France), which is also used for morphometric studies. En face quantification is used for atherosclerotic plaques along the thoracoabdominal aorta. The aorta is flushed with PBS through the left ventricle and removed from the root to the iliac bifurcation. Then, the aorta is fixed with 10% neutral-buffered formalin. After a thorough washing, adventitial tissue is removed, and the aorta opened longitudinally to expose the luminal surface. Afterward, the aorta is stained with Oil Red O for visualizing with the atherosclerotic lesions quantified by a blinded operator. Collagen is detected using Sirius red stain, and necrotic core is quantified after Masson's trichrome staining. Macrophage presence is determined using specific antibodies. At least 4 sections per mouse are examined for each immunostaining, and appropriate negative controls are used. For immunostaining of mouse atherosclerotic plaques, antibodies against Trem-1 (Bs 4886R), macrophage/monocyte antibody (MOMA)-2 (specifically MAB1852), Ly6G, (1A8), and CD3 (A0452) are used. Terminal dUTP nick end-labeling (TUNEL) staining is performed using histochemistry and fluorescent staining. Total proteins are extracted from human atherosclerotic plaque, and TREM-1 protein level is quantified by Luminex (Thermo Fischer Scientific).

Cells are cultured in RPMI 1640 medium supplemented with L-alanyl-L-glutamine dipeptide (Glutamax, Thermo Fisher Scientific), 10% fetal calf serum, 0.02 mM b-mercaptoethanol, and antibiotics. For cytokine measurements, splenocytes are stimulated with lipopolysaccharide (LPS) (10 μg/ml) and interferon (IFN)-g (100 UI/ml) for 24 or 48 h. IL-10, IL-12, and TNF-α production in the supernatants is measured using specific enzyme-linked immunosorbent assays (ELISA).

Primary macrophages are derived from mouse bone marrow-derived cells (BMDM). Tibias and femurs of C57B16/J male mice are dissected, and their marrow is flushed out. Cells are grown for 7 days at 37° C. in a solution of RPMI 1640 medium, 20% neonatal calf serum, and 20% macrophage-colony-stimulating factor-rich L929-conditioned medium. To analyze oxidized LDL (oxLDL) uptake, BMDMs are exposed to human oxLDL (25 μg/ml) for 24 and 48 h. Cells are washed, fixed, and stained using Red Oil. Foam cells are quantified blindly on 6 to 8 fields, and the mean is recorded. To analyze macrophage phenotype, BMDMs are stimulated with LPS (10 μg/ml) and IFN-g (100 UI/ml) for 24 h. IL-10, IL-12, IL-1b, and TNF-α production in the supernatant is measured using ELISA. To analyze apoptosis susceptibility, macrophages are incubated with TNF-α (10 ng/ml) and cycloheximide (10 μmol/l) for 6 h or etoposide (50 μmol/l) for 12 h, or in a fetal calf serum-free medium. Apoptosis is determined by independent experiments using Annexin V fluorescein isothiocyanate apoptosis detection kit with 7-AAD (APC, BD Biosciences, San Jose, Calif.) according to the manufacturer's instructions.

Human monocytes are isolated using anti-CD14 microbeads from healthy donors. Cells are cultured with macrophage colony-stimulating factor (50 ng/ml) for 7 days to induce mature macrophages. Nonclassical monocytes are labeled in vivo by retro-orbital intravenous injection of 1 mm fluorescent microsphere diluted to one-quarter in sterile PBS. Chimeric Ldlr^{-/-} [symbol] mice were euthanized 48 h later, and cell labeling is checked by flow cytometry. Beads that reflect monocyte recruitment are quantified in 8 aortic sinus sections per mouse.

### Statistical Analysis

Values are mean±SE of the mean. Differences between values are examined using the nonparametric Mann-Whitney U test and are considered significant at a p value of <0.05.

### Example 18A: Modulation of the TREM-1 Pathway in a Mouse Model of DSS-Induced Colitis and Colitis-Associated Tumorigenesis

In order to demonstrate that the TREM-1-related TRIOPEP formulations are effective in inhibiting TREM-1-mediated cell activation, decreasing intestinal epithelial proliferation in dextran sulfate sodium (DSS)-induced colitis and ameliorating the development of inflammation and tumor within the colon through exerting anti-inflammatory effects, the experiments can be conducted analogously to those described in Zhou, et al. Int Immunopharmacol 2013, 17:155-161.

### Animals and DSS-Induced Colitis and Colitis-Associated Tumorigenesis

C57BL/6 mice are purchased from Zhejiang Provincial Laboratories and (aged 8 to 12 weeks) maintained in a specific pathogen-free facility. Mice are treated with 7 days of 3.5% DSS (MP Biomedicals) in regular drinking water. To develop colitis-associated tumors, mice are first injected with 10 mg/kg azoxymethane (AOM) (Sigma-Aldrich) intraperitoneally (i.p.) followed 5 days later by a 5 day course of 2% DSS. Mice are then allowed to recover for 16 days with regular drinking water. The cycle of five days of 2% DSS followed by 16 days of regular drinking water is repeated twice. Mice are sacrificed 21 days after the last cycle of DSS for tumor counting. Colons are harvested, flushed of feces and longitudinally slit open to grossly count tumors with the aid of a magnifier and stereomicroscope.

### Treatments

Starting on day 0 (at the beginning of colitis induction), mice are treated once daily with either G-HV21, G-KV21 or G-TE21 at various concentrations injected i.p. in 200 μl saline. To investigate the effects of blocking TREM-1 after induced inflammation, colitis is induced by 4% DSS for 4 days. After colitis induction, mice are administered with either G-HV21, G-KV21 or G-TE21 for the next 5 days.

### Quantitative RT-PCR

Total RNA from colons is collected after colon tissue homogenization using the Trizol (Pierce). cDNA is synthesized using iScript (MBI) and then used in quantitative PCR reactions with SYBR Green using specific primers: TNF-alpha forward 5′-AGGCTGCCC CGACTACGT-3′ and reverse 5′-GACTTTCTCCTGGTATGAGATAGCAAA-3′; IFN-gamma forward 5′-CAGCAACAGCAAGGCGAAA-3′ and reverse 5′-CTGGACCTGTGGGTTGTT GAC-3′; IL-1beta forward 5′-TCGCTCAGGGTCACAAGAAA-3′ and reverse 5′-CATCAGAGGCAAGGAGGAAAAC-3′; IL-6 forward 5′-ACAAGTCGGAGGCTTAATTACACAT-3′ and reverse 5′-ATGTGTAATTAAGCCTCCGACTTGT-3′; IL-17 forward 5′-GCTCCAGAA GGCCCTCAGA-3′ and reverse 5′-AGCTTTCCCTCCGCATTGA-3′; macrophage inflammatory protein-2 (MIP-2) forward 5′-CACTCTCAAGGGCGGTCAA-3′ and reverse 5′-AGGCACATCAGGTACGATCCA-3′; 3-actin forward 5′-AGATTACTGCTCTGGCTC CTA-3′ and reverse 5′-CAAAGAAAGGGT GTAAAACG-3′. Relative expression levels of mRNA are normalized to β-actin. PCR products are separated on a 1.5% agarose gel and stained with ethidium bromide. Relative quantification of mRNA is performed by densitometry using QuantityOne software (Biorad Laboratories). Reactions are performed on the ABI 7900HT.

### ELISA

The serum levels of TNF-alpha, IL-1beta and IL-6 are measured using the specific ELISA kits (R&D Systems) following the manufacturer's instructions. All samples are ran in duplicate and analyzed on the same day.

### Evaluation of Inflammation

Colons are harvested from mice, flushed free of feces and jelly-rolled for formalin fixation and paraffin embedding. 5 m sections are used for hematoxylin and eosin staining. Histologic assessment is performed in a blinded fashion using a scoring system. A 3-4 point scale is used to denote the severity of inflammation (0=none, 1=mild, 2=moderate, and 3=severe), the level of involvement (0=none, 1=mucosa, 2=mucosa and submucosa and 3=transmural) and extent of epithelial/crypt damage (0=none, 1=basal 1/3, 2=basal 2/3, 3=crypt loss, and 4=crypt and surface epithelial destruction). Each parameter is then multiplied by a factor reflecting the percentage of the colon involved (0-25%, 26-50%, 51-75%, and 76-100%), and then summed to obtain the overall score. Assessment of colon weight after DSS treatment is performed by measuring the weight of colons (excluding the cecum) after removal of feces and normalizing by the length of colon in age- and sex-matched mice.

### Intestinal Permeability

Mice are fasted for 4 h with the exception of drinking water prior to the administration of 0.6 mg/kg FITC-dextran (4 kD, Sigma). Serum is collected 4 h later retro-orbitally, diluted 1:3 in PBS and the amount of fluorescence is measured using a fluorescent spectrophotometer with emission at 488 nm, and absorption at 525 nm.

### Intestinal Epithelial Proliferation

Mice are injected with 100 mg/kg BrdU (B.D. Pharmingen) i.p. 2.5 h prior to sacrifice at various time points after treatment with AOM/DSS. Colons are then dissected free, flushed free of feces, jelly-rolled, formalin-fixed, and paraffin-embedded. Sections are subsequently stained using the BrdU (BD Biosciences).

### Apoptosis

Colon sections from formalin-fixed, paraffin-embedded tissues are assessed for apoptotic cells using the ApoAlert DNA fragmentation assay kit (Clontech).

### Statistics

Data are presented as mean±SEM. Survival curves is assessed by log-rank test. The tumor counts, intestinal permeability, cytokine measurements, proliferation and apoptosis levels between mice treated with either G-HV21, G-KV21 or G-TE21 are compared using the Student's unpaired t-test. p<0.05 is considered statistically significant.

### Example 19A: Synthesis and Modification of Paclitaxel-Conjugated Peptides

This example demonstrates one embodiment of a synthesized trifunctional peptide compound containing PTX (PTX/TRIOPEP). Exemplary use of TREM-1/TRIOPEP G-KV21 is described.

The first step is to synthesize the trifunctional compound comprising domains A and B where domain A is paclitaxel (PTX) bound to TREM-1 inhibitory peptide sequence GF9 (GFLSKSLVF), whereas domain B is a 12 amino acids-long peptide sequence WQEEMELYRQKV with either unmodified or modified amino acid residue(s) (see TABLE 2). Although it is not necessary to understand the mechanism of an invention, it is believed that as an anticancer agent, PTX may exhibit not only its microtubule-stabilizing activity, but also its ability to stimulate release of anticancer cytokines from tumor-associated macrophages (TAMs) and functions to treat and/or prevent a cancer-related disease or condition alone or synergistically with the domain A sequence GF9, whereas a 12 amino acids-long peptide sequence WQEEMELYRQKV with either unmodified or modified amino acid residue(s) functions to mediate formation of naturally long half-life LP upon interaction with native lipoproteins and to target the formed particles to cancer cells and/or TAMs, respectively.

In one embodiment, the trifunctional peptide compound comprises domains A and B where domain A is PTX is conjugated to TREM-1 inhibitory peptide sequence GFLSKSLVF, whereas domain B is a 12 amino acids-long peptide sequence GEEMRDRARAHV with either unmodified or sulfoxidized methionine residue (see TABLE 2).

In one embodiment, PTX is conjugated to the acetylated 21 amino acids-long sequence of TREM-1/TRIOPEP where the domain A comprises acetylated peptide sequence GFLSKSLVF whereas domain B comprises sequence GEEMRDRARAHV (i.e. PTX-Gly-Phe-Leu-Ser-Lys-Ser-Leu-Val-Phe-Gly-Glu-Glu-Met-Arg-Asp-Arg-Ala-Arg-Ala-His-Val-OH or PTX-GFLSKSLVFGEEMRDRARAHV), hereafter referred to as a PTX/TREM-1-related “TRIOPEP” peptide compound or “PTX-TREM-1/TRIOPEP G-KV21”.

Peptides can be synthesized or purchased from specialized companies (i.e., Sigma-Genosys, Woodlands, Tex., USA) with greater than 95% purity as assessed by HPLC. Peptide molecular mass can be checked by matrix-assisted laser desorption ionization mass spectrometry. The trifunctional peptide compounds containing conjugated PTX can be synthesized analogously as described in Lin et al. Chem Commun (Cambridge) 2013; 49:4968-4970 and disclosed in Castaigne, et al. U.S. Pat. No. 9,173,891.

### Synthesis of 4-(Pyridin-2-Yldisulfanyl) Butyric Acid

4-Bromobutyric acid (2 g, 12 mmol) and thiourea (0.96 g, 12.6 mmol) are dissolved in ethanol (50 mL) and refluxed at 90° C. for 4 h. After dropwise addition of a NaOH solution (4.8 g in 5:1 H2O/ethanol), the mixture is refluxed for another 16 h and then cooled to room temperature. The white precipitate is collected and redissolved in water (40 mL). 4 M HCl is used to adjust the solution pH to 5, and the product is extracted into diethyl ether. The organic phase is dried over anhydrous sodium sulfate to give 4-sulfanylbutyric acid as a colorless oil (310 mg, 15%), which is used in the next step without further purification. 4-sulfanylbutyric acid (105 mg, 0.87 mmol) and 2-aldrithiol (440 mg, 2.0 mmol, 2.3 eq) are dissolved in MeOH (1.3 mL) and stirred for 3 h. The solution is purified by RP-HPLC (5% to 95% of acetonitrile in water with 0.1% TFA over 45 min), combining product fractions and removing solvents to give 4-(pyridin-2-yldisulfanyl) butyric acid as an oil (118 mg, 59%).

### Paclitaxel C2′ Ester Synthesis

Paclitaxel (186 mg, 0.22 mmol), 4-(pyridin-2-yldisulfanyl)butyric acid (100 mg, 0.44 mmol), N,N′-diisopropylcarbodiimide (DIC) (68 μL, 0.44 mol), and 4-dimethylaminopyridine (DMAP) (26.7 mg, 0.22 mmol) are added into an oven dried flask equipped with a stirrer bar, evacuated and refilled with nitrogen three times to remove air, then dissolved in anhydrous acetonitrile (12.7 mL). The reaction is allowed to stir in the dark at room temperature for 48 h. The solvents are removed under vacuum and the residue is dissolved in chloroform and purified by flash chromatography (3:2 EtOAc/hexane), to give the product as a white solid (108 mg, 47%).

Synthesis of PTX-TREM-1/TRIOPEP (e.g. G-KV21) in Free and SLP-Bound Form.

GFLSKSLVFGEEMRDRARAHV (89.8 mg, 34.3 umol) and paclitaxel C2′ ester (54.7 mg, 51.4 umol) are added to an oven dried flask equipped with a stirrer bar and evacuated and filled with nitrogen three times to remove the air. The reagents are then dissolved in anhydrous dimethyl formamide DMF (5 mL). The solution is allowed to stir for 16 h, before purification by RP-HPLC (30% to 95% acetonitrile in water with 0.1% TFA over 45 min). Product fractions are combined and lyophilized to give a PTX-TREM-1/TRIOPEP G-KV21 as a white powder. Discoidal and spherical PTX-TREM-1/TRIOPEP-containing SLP are prepared, purified and characterized using the methods and procedures described herein in the Example 2.

### Example 20A: Use of PTX-TREM-1/TRIOPEP in Experimental Cancer

In order to demonstrate the anticancer activity of PTX-TREM-1/TRIOPEP, the experiments can be conducted analogously to those disclosed herein and described in Lin et al. Chem Commun (Cambridge) 2013; 49:4968-4970; Sigalov. Int Immunopharmacol 2014, 21:208-219; Shen and Sigalov. Mol Pharm 2017, 14:4572-4582; and disclosed in Castaigne, et al. U.S. Pat. No. 9,173,891.

### Cytotoxicity

The methyl thiazol tetrazolium (MTT) assay can be used to assess the cytotoxic effect of the PTX-TREM-1/TRIOPEP on cancer cells. The PTX-TREM-1/TRIOPEP may contain either unmodified or modified amino acid residue(s). Briefly, cells are plated in 96-well plates (5000 cells/well) in their respective media. Next day, the monolayers are washed with PBS (pH 7.4) twice, and then incubated at 37° C. for 24 h with the PTX-TREM-1/TRIOPEP in serum-free media. The following day, 25 μl of MTT (1 mg/ml) is added to each well and incubated for 3 h at 37° C. Plates are centrifuged at 1200 rpm for 5 min. The medium is removed, the precipitates are dissolved in 200 μl of DMSO and the samples are read at 540 nm in a microtiter plate reader.

### Animal Toxicity

Female C57BL6 mice (6-8 weeks, 18-21 g) can be used in toxicity studies of PTX-TREM-1/TRIOPEP. PTX-TREM-1/TRIOPEP may contain either unmodified or oxidized methionine residue. Groups of six mice each receives injections of 1.5 ml of PBS via the intraperitoneal route, containing respective doses of 30 mg/kg and 40 mg/kg of Taxol®, 40 mg/kg and 70 mg/kg of Abraxane® and different doses of PTX-TREM-1/TRIOPEP. The injections are administered on days 1, 2 and 3. A control group is injected with the vehicle. The weights and the health of the mice are monitored for 30 days. Weight measurements are performed once a day for the first 7 days and twice a week for the remaining monitoring period.

### Screening for PTX-TREM-1/TRIOPEP Incorporation

Cultured cells are incubated with PTX-TREM-1/TRIOPEP labeled with ¹⁴C-PTX (either pre-incubated or not with HDL). Subsequent to the incubation period, cells are trypsinized and the radioactivity of the lysate is determined to measure the extent of incorporation of the PTX into the cells.

### Tumor Suppression

Tumor suppression studies using PTX-TREM-1/TRIOPEP can be performed in animal models of cancer similarly as described above. Female 6-8 week old NU/J mice can be obtained from the Jackson Laboratory (Bar Harbor, Me.) Human cancer cell lines including but not limited to human carcinoma, human pancreas or human breast cancer cell lines can be obtained from ATCC. Tumor cells in culture are harvested and resuspended in a 1:1 ratio of RPMI 1640 and Matrigel (BD Biosciences, San Jose, Calif.). Human cancer xenografts are established by injecting subcutaneously into the right flanks certain amounts of viable cells per mouse. Tumor volumes are calculated with caliper measurements using the formula V=π/6 (length×width×width). When tumor grows to approximately 125 mm³ (100-150 mm³), animals are pair-matched by tumor size into treatment and control groups. Either PTX (TAXOL®; 30 mg/kg PTX) or PTX-TREM-1/TRIOPEP at different doses are intravenously administered to the animals via tail vein. Clinical observations, body weights and tumor volume measurements are made twice a week once tumors become measureable. It should be noted that TAXOL® is formulated with a detergent Cremophor that in itself is cytotoxic and is also the source of numerous side effects during chemotherapy. The Cremophor content of TAXOL® is about 80× that of paclitaxel per ml.

### Example 21A: Modulation of the TREM-1 Pathway in Experimental Arthritis

In order to demonstrate that TREM-1-related TRIOPEP formulations are effective in inhibiting TREM-1-mediated cell activation and protecting against bone and cartilage damage in animal models of rheumatoid arthritis (RA), the experiments were conducted as described in Shen and Sigalov. J Cell Mol Med 2017, 21:2524-2534.

### Chemicals, Lipids and Cells

Sodium cholate, cholesteryl oleate and other chemicals were purchased from Sigma-Aldrich Company (St. Louis, Mo., USA). 1,2-dimyristoyl-sn-glycero-3-phosphocholine (DMPC), 1,2-dimyristoyl-sn-glycero-3-phospho-(10-rac-glycerol) (DMPG), 1-palmitoyl-2-oleoyl-sn-glycero-3-phosphocholine (POPC), 1-palmitoyl-2-oleoyl-sn-glycero-3-phospho-(10-rac-glycerol) (POPG), 1,2-dimyristoyl-sn-glycero-3-phosphoethanolamine-N-(lissamine rhodamine B sulfonyl) (Rho B-PE) and cholesterol were purchased from Avanti Polar Lipids (Alabaster, Ala., USA). The murine macrophage cell line J774A.1 was obtained from the American Type Culture Collection (ATCC, Manassas, Va., USA).

### Animals

All animal experiments were performed in strict accordance with the recommendations in the Guide for the Care and Use of Laboratory Animals of the National Institutes of Health (NIH) and in the United States Department of Agriculture (USDA) Animal Welfare Act (9 CFR, Parts 1, 2, and 3).

### Collagen-Induced Arthritis (CIA) Model

Animal studies were performed by Bolder BioPATH (Boulder, Colo., USA). CIA was induced in male 6- to 7-week-old DBA/1 mice by immunization with bovine type II collagen. Briefly, mice were injected intradermally with 100 μl of Freund's complete adjuvant containing 250 μg of bovine type II collagen (2 mg/ml final concentration) at the base of the tail on day 0 and again on day 21. On day 24, mice were randomized by body weight into treatment groups. At enrolment on day 24, the mean mouse weight was 20 g. Arthritis onset occurred on days 26-38. Starting day 24, mice were injected i.p. intraperitoneally daily for 14 consecutive days with 5 mg/kg G-KV21, G-HV21, G-TE21, M-TK32 and M-VE32 or with PBS (vehicle). Mice were weighed on study days 24, 26, 28, 30, 32, 34, 36 and 38 (prior to necropsy). Daily clinical scores were given on a scale of 0-5 for each of the paws on days 24-38. On day 38, mice were killed for necropsy.

### Histology Assessment of Joints

At the end of study, fore paws, hind paws and knees were harvested, fixed in 10% neutral buffered formalin for 1-2 days, and then decalcified in 5% formic acid for 4-5 days before standard processing for paraffin embedding. Sections (8 μm) were cut and stained with toluidine blue (T blue). Hind paws, fore paws and knees were embedded and sectioned in the frontal plane. Six joints from each animal were processed for histopathological evaluation. The joints were then assessed using 0-5 scale for inflammation, pannus formation, cartilage damage, bone resorption and periosteal new bone formation. A summed histopathology score (sum of five parameters, 0-25 scale) was also determined.

### Cytokine Detection

Plasma was collected on days 24, 30 and 38, and cytokines were analysed by Quantibody Mouse Cytokine Array Q1 kits (RayBiotech, Norcross, Ga., USA) according to the manufacturer's instructions.

### Statistical Analysis

All statistical analyses were performed with GraphPad Prism 6.0 software (GraphPad, La Jolla, Calif., USA). Results are expressed as the mean±SEM. Statistical differences were analyzed using analysis of variance with Bonferroni adjustment. P values less than 0.05 were considered significant.

This example demonstrates that TREM-1/TRIOPEP G-HV21 and G-KV21 but not TREM-1-related control peptide G-TE21 ameliorate CIA and protect against bone and cartilage damage. This example further demonstrates that TCR/TRIOPEP M-VE32 but not TCR-related control peptide M-TK32 ameliorates CIA and protects against bone and cartilage damage. This example further demonstrates that TREM-1/TRIOPEP and TCR/TRIOPEP peptides are non-toxic and well-tolerable by arthritic mice. See FIG. 42A-B.

### Example 22A: Modulation of the TREM-1 Pathway in Experimental Retinopathy

In order to demonstrate that TREM-1-related TRIOPEP are effective in inhibiting TREM-1-mediated cell activation and reducing pathological retinal neovascularization (RNV), the experiments were conducted as described in Rojas, et al. Biochim Biophys Acta 2018, 1864:2761-2768.

### Mouse Model of Oxygen-Induced Retinopathy (OIR)

This study was carried out in strict accordance with the recommendations in the Guide for the Care and Use of Laboratory Animals of the National Institutes of Health and in the United States Department of Agriculture (USDA) Animal Welfare Act (9 CFR, Parts 1, 2, and 3). animals were treated according to the ARVO Statement for the Use of Animals in Ophthalmic and Vision Research.

Litters of C57BL/6J (Jackson Laboratory, Bar Harbor, Me.) neonatal mice and nursing dams were exposed to a hyperoxia environment (75% oxygen) from postnatal day 7 (P7) to P12 and returned to normoxia until P17. The hyperoxia exposure causes degeneration of the immature retinal vessels. This results in severe hypoxia upon return to the normoxia environment which leads to vitreoretinal neovascularization. Beginning on P7, mice were treated until day P17 by daily i.p. injections of G-KV21, G-TE21 at indicated doses or vehicle (PBS, pH 7.4). In certain embodiments, neonatal mice and nursing dams were not subjected to a hyperoxia environment and reared in room air. At P17, all mice were humanely sacrificed and their retinas were collected. In one embodiment, i.p. administered rho B-labeled G-KV21 was used to confirm its ability to cross the BRB in mice. In one embodiment, i.p. administered rho B-labeled G-KV21 was used to confirm its ability to cross the BBB in rats and rabbits.

### Immunofluorescence Staining

Treatment effects on vaso-obliteration and pathological angiogenesis were assessed by morphometric analysis of the avascular and neovascularization areas in retinal flat mounts after labeling with isolectin B₄ as described in Patel, et al. Am J Pathol 2014, 184:3040-3051. Immunofluorescence analysis (IFA) of the retina flat mounts was performed to assess the effects of the TREM-1-targeting treatments on the distribution of TREM-1, M-CSF and markers for inflammatory cells (CD45) and activated macrophage/microglial cells (Iba-1) in relation to RNV. Retinal frozen sections from pups kept in RA and from the OIR pups were fixed in 4% paraformaldehyde for 15 min (or in cold acetone at −20° C. for 30 min), washed 3 times with PBS, and blocked with a solution containing 0.3% Triton X and 3% normal goat serum (NGS) for 30 min. Then, the samples were reacted with a rat anti-mouse TREM-1 antibody (Abcam, Cambridge, Mass.), rabbit polyclonal anti-mouse M-CSF antibodies (Abcam, Cambridge, Mass.), rabbit polyclonal anti-mouse CD45 antibodies (Santa Cruz Biotechnology, Dallas, Tex.), a rabbit anti-mouse Iba-1 antibody (Wako Chemical USA, Inc.), and kept at 4° C. overnight. Then, the samples were washed 3 times with PBS and stained with a donkey-anti-rat Oregon green antibody for TREM-1, a goat anti-rabbit Texas red antibody for CD45 and Iba-1 or a donkey anti-rabbit Texas red antibody for M-CSF (Invitrogen, Waltham, Mass.). After washing 3 times with PBS, the images were captured with a 20× lens using a Zeiss Axioplan2 fluorescence microscope (Carl Zeiss Meditec, Inc., Dublin, Calif.). Intravitreal neovascular formation and avascular area were measured as described in Connor, et al. Nat Protoc 2009, 4:1565-1573.

### Western Blot Analysis

Retina samples from OIR-treated and room air control pups were homogenized in the modified RIPA buffer (20 mM Tris-HCl, 2.5 mM EDTA, 50 mM NaF, 10 mM Na₄P₂O₇, 1% Triton X-100, 0.1% sodium dodecyl sulfate, 1% sodium deoxycholate, 1 mM phenyl methyl sulfonyl fluoride, pH 7.4). Samples containing equal amounts of protein were separated by 12% sodium dodecyl sulfate polyacrylamide gel electrophoresis, transferred to nitrocellulose membrane, and reacted for 24 hrs with monoclonal rat anti-mouse TREM-1 or polyclonal rabbit M-CSF antibodies (Abcam, Cambridge, Mass.) in 5% milk, followed by incubation with corresponding horseradish peroxidase-linked secondary antibodies (GE Healthcare Bio-Science Corp., Piscataway, N.J.). Bands were quantified by densitometry, and the data were analyzed using ImageJ software and normalized to loading control. Equal loading was verified by stripping the membranes and reprobing them with a monoclonal antibody against β-actin (Sigma-Aldrich, St Louis, Mo.).

### Statistical Analysis

Group differences were compared by one-way ANOVA followed with a post hoc test for multiple comparisons. Values are represented as the means±standard error of the means (SEM). Results were considered statistically significant when P≤0.05.

This example demonstrates that TREM-1/TRIOPEP G-KV21 but not TREM-1-related control peptide G-TE21 significantly (up to 95%) reduces pathological RNV in a mouse model of retinopathy. It further that demonstrates that TREM-1/TRIOPEP is non-toxic and well-tolerated in mouse litters. TREM-1 inhibition substantially downregulates retinal protein levels of TREM-1 and M-CSF suggesting that TREM-1-dependent suppression of pathological angiogenesis involves M-CSF. This example further demonstrates that sSLP and TREM-1/TRIOPEP-sSLP pass the blood-retinal barrier (BRB) and blood-brain barrier (BBB). See FIGS. 29A-C.

### Example 23A: Modulation of the TREM-1 Pathway in Experimental Alcoholic Liver Disease (ALD)

In order to demonstrate that TREM-1-related TRIOPEP formulations are effective in inhibiting TREM-1-mediated cell activation and ameliorating ALD, the experiments can be conducted in the Lieber DeCarli ALD mouse model as described in Petrasek, et al. J Clin Invest 2012, 122:3476-3489, herein incorporated by reference in it's entirety.

### Animals

C57BL/6 female mice (10- to 12-week-old) are purchased from The Jackson Laboratory (Bar Harbor, Me., USA). Mice (n=6-9/group) are acclimated to a Lieber-DeCarli liquid diet of 5% ethanol (vol/vol) over a period of 1 week, then maintained on the 5% diet for 4 weeks. Pair-fed control mice are fed a calorie-matched dextran-maltose diet. All animals have unrestricted access to water throughout the entire experimental period. In treated groups, mice are i.p. treated 5 days/week with vehicle (PBS), G-KV21, G-HV21 or G-TE21 from the first day on a 5% ethanol diet. At the end of all animal experiments, cheek blood samples are collected in serum collection tubes (BD Biosciences, San Jose, Calif., USA) and processed within an hour. After blood collections, mice are euthanized, and liver samples are harvested and stored at −80° C. until further analysis.

Total Protein Isolation from Liver

Total protein is extracted from liver samples using RIPA buffer (Boston Bio-products Cat. #BP-115) supplemented with protease inhibitor cocktail tablets (Roche Cat. #11836153001) and Phospho Stop phosphatase inhibitor (Roche Cat. #04906837001). Cell debris is then removed from cell lysates by 10 minutes centrifugation at 2000 rpm.

### Biochemical Assays and Cytokines

Serum alanine aminotransferase (ALT) levels are determined by kinetic method using commercially available reagents from Teco Diagnostics (Anaheim, Calif., USA). Liver triglycerides are extracted using a 5% NP-40 lysis solution buffer and quantified using a commercially available kit (Wako Chemicals, Richmond, Va., USA) followed normalization to protein amount analyzed by Pierce BCA protein assay (Thermo Scientific, Rockford, Ill., USA). Cytokine levels are measured in serum samples and whole liver lysates diluted in assay diluent following the manufacturer's instructions. Specific anti-mouse ELISA kits are used for the quantification of MCP-1, TNFα (BioLegend Inc., San Diego, Calif., USA) and IL-1β (R&D Systems, Minneapolis, Minn., USA) levels. For normalization, the total protein concentration of the whole liver lysate is determined using Pierce BCA protein assay.

### Western Blot Analysis

Whole liver proteins are boiled in Laemmli's buffer. The samples are resolved in 10% SDS-PAGE gel under reducing conditions using Tris-glycine buffer system and resolved proteins are transferred onto a nitrocellulose membrane. SYK proteins are detected by specific primary antibodies (SYK: 2712—Cell Signaling and phospho-SYKY525/526: ab58575—Abcam) followed by an appropriate secondary HIRP-conjugated IgG antibody from Santa Cruz Biotechnology. β-actin, detected by an ab49900 antibody (Abcam), is used as a loading control. The specific immunoreactive bands of interest are visualized by chemiluminescence (Bio-Rad) using the Fujifilm LAS-4000 luminescent image analyzer.

### RNA Extraction and Quantitative Real-Time PCR Analysis

Total RNA is extracted using the Qiagen RNeasy kit (Qiagen) according to the manufacturer's instructions with on-column DNase treatment. RNA is quantified using a Nanodrop 2000 spectrophotometer (Thermo Scientific) and cDNA synthesis is performed using the iScript Reverse Transcription Supermix (Bio-Rad Laboratories) and 1 μg total RNA. Real-time quantitative PCR is performed using Bio-Rad iTaq Universal SYBR Green Supermix (Bio-Rad Laboratories) and a CFX96 real-time detection system (Bio-Rad Laboratories). Relative gene expression is calculated by the comparative ΔΔCt method. The expression level of target genes is normalized to the house-keeping gene, 18S rRNA, in each sample and the fold-change in the target gene expression between experimental groups is expressed as a ratio. Primers are synthesized by IDT, Inc. and exemplary sequences are listed Table 3B.

TABLE 3B
Mouse primers.

| | Primers | |
|---|---|---|
| Mouse | Forward sequence | Reverse sequence |
| primers | 5′ to 3′ | 5′ to 3′ |
| 18s | GTA ACCCGTTGAACC | CCATCCAATCGGTAGT |
| | CCATT | AGCG |
| TREM-1 | TCCTATTACAAGGCTG | AAGACCAGGAGAGGAA |
| | ACAGAGCGTC | ACAACCGC |
| TNF-α | CACCAC CATCAA GG | AGGCAACCTGACCAC |
| | ACTC AA | TCTCC |
| MCP-1 | CAGGTCCCT GTCATG | CAGGTCCCTGTC ATG |
| | CTTCT | CTTCT |
| IL-1β | CTTTGAAGTTGACGGA | TGAGTGATACTGCCTG |
| | CCC | CCTG |
| MPO | CATCCAACCCTTCATG | CTGGCGATTCAGTTTG |
| | TTCC | G |
| LY6G | TGCGTTGCTCTGCTGG | CAGAGTAGTGGGGCAG |
| | AGATAGA | ATGG |
| F4/80 | TGCATCTAGCAATGGA | GCCTTCTGGATCCATT |
| | CAGC | TGAA |
| CD68 | TGTCTGATCTTGCTAG | GAGAGTAACGGCCTTT |
| | GACCG | TTGTG |
| Pro- | GCTCCTCTTAGGGGCC | CCACGTCTCACCATTG |
| Collagen1α | ACT | GG |
| α-SMA | GTCCCAGACATCAGGG | TCGGATACTTCAGCGT |
| | AGTAA | CAGGA |
| ACC1 | AGCAGATCCGCAGCTT | ACCTCTGCTCGCTGAG |
| | G | TGC |
| MIP-1α | TTCTCTGTACCATGAC | GCATTAGCTTCAGATT |
| | ACTCTGC | TACGGGT |
| RANTES | GCTGCTTTGCCTACCT | TCGAGTGACAAACACG |
| | CTCC | ACTGC |
| ADRP | CTGTCTACCAAGCTCT | CGATGCTTCTCTTCCA |
| | GCTC | CTCC |
| PPARα | AACATCGAGTGTCGAA | AGCCGAATAGTTCGCC |
| | TATGTGG | GAAAG |
| SREBF1 | GCTTCTTACAGCACAG | TTTCATGCCCTCCATA |
| | CAACC | GACAC |
| CPT1A | CCAGGCTACAGTGGGA | GAACTTGCCCATGTCC |
| | CATT | TTGT |
| MCAD/ | GATCGCAATGGGTGCT | AGCTGATTGGCAATGT |
| MACD | TTTGATAGAA | CTCCAGCAAA |

### Liver Histopathology

Sections of formalin-fixed, paraffin-embedded liver specimens from mice are stained with Hematoxylin/Eosin (H&E) or F4/80 (ThermoFisher, Cat #MF48000), MPO (Abcam Cat #ab9535) antibodies for immunohistochemistry, the fresh frozen samples are stained with Oil-Red-O at the UMMS DERC histology core facility.

### Statistical Analysis

All statistical analyses are performed using GraphPad Prism 7.02 (GraphPad Software Inc.). Significance levels are determined using one way analysis of variance (ANOVA) followed by a post hoc test for multiple comparisons. Data are shown as mean±SEM and differences were considered statistically significant when p<0.05.

### Example 24A: Synthesis of Imaging Probe ([⁶⁴Cu])-Conjugated TRIOPEP Peptides

This example demonstrates one embodiment of a synthesized TREM-1-related trifunctional peptide compound containing imaging probe [⁶⁴Cu] ([⁶⁴Cu]TREM-1/TRIOPEP).

The first step is to synthesize the trifunctional compound comprising domains A and B where domain A is a TREM-1 inhibitory peptide sequence GF9 (GFLSKSLVF), whereas domain B is either a [⁶⁴Cu]-labeled 12 amino acids-long 12 amino acids-long peptide sequence WQEEMELYRQKV with sulfoxidized methionine residue or a [⁶⁴Cu]-labeled 12 amino acids-long peptide sequence GEEMRDRARAHV with sulfoxidized methionine residue. Although it is not necessary to understand the mechanism of an invention, it is believed that 12 amino acids-long peptide sequences with sulfoxidized methionine residues will mediate formation of naturally long half-life LP upon interaction with native lipoproteins and target these [⁶⁴Cu]TREM-1/TRIOPEP-containing particles to macrophages, whereas GF9 peptide sequence will assist in the self-insertion of [⁶⁴Cu]TREM-1/TRIOPEP released from [⁶⁴Cu]TREM-1/TRIOPEP-containing LP upon their endocytosis by macrophages (e.g., TAMs, Kupffer cells, etc.) into the cell membrane and subsequent colocalization of [⁶⁴Cu]TREM-1/TRIOPEP with TREM-1 expressed on TAMs. This is believed to result in TREM-1 inhibition along with [⁶⁴Cu]TREM-1/TRIOPEP-PET signal in the macrophage-rich areas of interest allowing for visualization of macrophage-mediated inflammation (e.g., neuroinflammation, inflamed atherosclerotic plaques, intratumoral inflammation, etc.).

In one embodiment, [⁶⁴Cu] is conjugated to the 21 amino acids-long sequence of TREM-1/TRIOPEP G-HV21 Ac-Gly-Phe-Leu-Ser-Lys-Ser-Leu-Val-Phe-Gly-Glu-Glu-Met-Arg-Asp-Arg-Ala-Arg-Ala-His-Val-OH (i.e., [⁶⁴Cu]GFLSKSLVFGEEMRDRARAHV), hereafter referred to as a [⁶⁴Cu]-related “TRIOPEP” peptide compound or “[⁶⁴Cu]/TRIOPEP”.

Peptides can be synthesized or purchased from specialized companies (i.e., Sigma-Genosys, Woodlands, Tex., USA) with greater than 95% purity as assessed by HPLC. Peptide molecular mass can be checked by matrix-assisted laser desorption ionization mass spectrometry. The trifunctional peptide compounds containing conjugated [⁶⁴Cu] can be synthesized analogously as disclosed in James and Andreasson, WO 2017083682A1.

DOTA (1,4,7, 10-tetraazacyclododecane-1,4,7,10-tetraacetic acid) conjugation is performed according to established protocols, using metal-free buffers. After conjugation, matrix-assisted laser desorption/ionization (MALDI) mass spectrometry is conducted to determine the average number of DOTA molecules conjugated per TREM-1/TRIOPEP. Subsequently, the DOTA-conjugated TREM-1/TRIOPEP is radiolabeled with [⁶⁴Cu] by incubating it in a [⁶⁴Cu]CuCl₂ solution (pH 5.5) at 37° C. for one hour with continual shaking. The reaction is purified via a NAP5 column and specific activity of the final labeled TREM-1/TRIOPEP is determined via size exclusion HPLC. [⁶⁴Cu]TREM-1/TRIOPEP can be synthesized with high specific radioactivity (>75 GBq/μΓTîoI), radiochemical purity (>99%), and labeling efficiency (50-75%), which is sufficient for in vitro and in vivo use.

### Example 25A: Use of [⁶⁴Cu]TREM-1/TRIOPEP in Imaging of Neuroinflammation

In one embodiment, in order to demonstrate the feasibility of using [⁶⁴Cu]TREM-1/TRIOPEP to visualize neuroinflammation in vivo, PET/CT imaging of middle cerebral artery occlusion (MCAo) mice can be performed analogously as disclosed in James and Andreasson, WO 2017083682A1.

The MCAo model of cerebral ischemia is selected since the time-course of macrophage infiltration and microglial activation in the brain infarct is well documented, and because this model is commonly used to evaluate candidate microglial/macrophage-PET tracers. B6 mice (n=3), MCAo (n=9), and sham (n=9) mice are injected via tail vein with 80-85μΩ of [⁶⁴Cu]TREM-1/TRIOPEP in a saline solution (0.9% sodium chloride) and imaged using PET/CT at 3 h post-injection. They are imaged again at 19 h post-injection, which is 1.5-2 days after surgery/stroke.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for treating cancer in a subject in need thereof, said method comprising administering to said subject a therapeutically effective amount of at least one peptide inhibitor for inhibiting activity of the TREM-1/DAP-12 signaling pathway.

2. The method of claim 1, wherein said therapeutically effective amount comprises one dose of said at least one peptide inhibitor.

3. The method of claim 2, wherein said therapeutically effective amount comprises between two to ten doses of said at least one peptide inhibitor.

4. The method of claim 1, wherein said at least one peptide inhibitor is the amino acid sequence GFLSKSLVF (GF9).

5. The method of claim 4, wherein said at least one peptide inhibitor is administered without recombinant high-density lipoprotein carriers.

6. The method of claim 1, wherein said at least one peptide inhibitor has a methionine sulfoxide M(O) modified amino acid residue.

7. The method of claim 1, wherein said at least peptide inhibitor is administered without recombinant high-density lipoprotein carriers.

8. The method of claim 1, wherein said at least one peptide inhibitor is administered with recombinant high-density lipoprotein carriers.

9. The method of claim 1, wherein said at least one peptide inhibitor has an amino acid sequence selected from the group consisting of
GFLSKSLVFPYLDDFQKKWQEEMELYRQKVE
and
GFLSKSLVFPLGEEMRDRARAHVDALRTHLA.

10. The method of claim 1, wherein said at least one peptide inhibitor has an amino acid sequence selected from the group consisting of
GFLSKSLVFPYLDDFQKKWQEEM(O)ELYRQKVE (GA31)
and
GFLSKSLVFPLGEEM(O)RDRARAHVDALRTHLA (GE31).

11. The method of claim 10, wherein said peptide inhibitor has equimolar amounts of peptide GA31 and peptide GE31.

12. The method of claim 1, wherein said at least one peptide inhibitor has an amino acid sequence selected from the group consisting of GFLSKSLVFGEEMRDRARAHV (G-HV21), GFLSKSLVFWQEEMELYRQKV (G-KV21), MWKTPTLKYFPYLDDFQKKWQEEMELYRQKVE (M-VE32), and mixtures thereof.

13. The method of claim 1, wherein said at least one peptide inhibitor has an amino acid sequence selected from the group consisting of GFLSKSLVFGEEM(O)RDRARAHV (G-HV21), GFLSKSLVFWQEEM(O)ELYRQKV (G-KV21), (M(O)WKTPTLKYFPYLDDFQKKWQEEM(O)ELYRQKVE (M-VE32), and mixtures thereof.

14. The method of claim 1, wherein said at least one peptide inhibitor is administered together with a therapeutically effective amount of a therapeutic selected from the group consisting of an anticancer vaccine, an anticancer immunotherapy agent, anti-cancer immunomodulatory agent, an additional anticancer therapeutic, radiation therapy, surgery, and any combination thereof.

15. The method of claim 1, comprising administering said at least one peptide inhibitor together with a pharmaceutically acceptable carrier selected from the group consisting of an excipient, diluent, and any combination thereof.

16. The method of claim 15, wherein said carrier is selected from the group consisting of lipids, proteins or polypeptides, and mixtures thereof.

17. The method of claim 2, wherein, prior to administering said first dose of said peptide inhibitor, said subject received a prior therapy selected from the group consisting of an anticancer vaccine, an anticancer immunotherapy agent, anti-cancer immunomodulatory agent, an additional anticancer therapeutic, radiation therapy, surgery or a combination thereof.

18. The method of claim 17, wherein said cancer recurred or progressed after said prior therapy.

19. The method of claim 1, wherein said administering is continued as a maintenance treatment for duration between two weeks to five years.

20. The method of claim 1, wherein said administration is continued for a duration of up to one year.

21. The method of claim 14, wherein said anticancer vaccine is selected from the group consisting of Gardasil, Cervarix, and Sipuleucel-T/Provenge.

22. The method of claim 14, wherein the anticancer immunotherapy agent is selected from the group consisting of Alemtuzumab, Ipilimumab, Ofatumumab, Nivolumab, Pembrolizumab, Rituximab, Blinatumomab, Daratumumab, Trastuzumab, Cetuximab, Elotuzumab, adoptive T-cell therapy, T-Vec, Interferon, Interleukin, and any combination thereof.

23. The method of claim 14 wherein the anticancer immunomodulatory agent is selected from the group consisting of thalidomide, lenolidomide, pomalidomide, and any combination thereof.

24. The method of claim 14, wherein the additional anticancer therapeutic is selected from the group consisting of an alkylating agent, a tubulin inhibitor, a topoisomerase inhibitor, proteasome inhibitor, a CHK1 inhibitor, a CHK2 inhibitor, a PARP inhibitor, a tyrosine kinase inhibitor, CSF-1/CSF-1R inhibitor, doxorubicin, gemcitabine, entrectinib, epirubicin, vinblastine, etoposide, topotecan, bleomycin, and mytomycin c.

25. The method of claim 24, wherein said alkylating agent is selected from the group consisting of Dacarbazine, Procarbazine, Carmustine, Lomustine, Uramustine, BuSulfan, Streptozocin, Altreamine, Ifosfamine, Chrormethine, Cyclophasphamide, Cyclophosphamide, Chlorambucil, Fluorouracil (5-Fu), Melphalan, Triplatin tetranitrate, Satraplatin, Nedaplatin, Cisplatin, Carboplatin, and Oxaliplatin.

26. The method of claim 24, wherein said tubulin inhibitor is selected from the group consisting of Taxol, Docetaxel, Abraxane, Vinblastin, Epothilone, Colchicine, Cryptophycin, BMS 347550, Rhizoxin, Ecteinascidin, Dolastin 10, Cryptophycin 52, and IDN-5109.

27. The method of claim 24, wherein said topoisomerase inhibitor is a topoisomerase I inhibitor selected from the group consisting of Irinotecan, Topotecan, and Camptothecins (CPT).

28. The method of claim 24, wherein said topoisomerase inhibitor is a topoisomerase II inhibitor selected from the group consisting of Amsacrine, Etoposide, Teniposide, Epipodophyllotoxins, and ellipticine.

29. The method of claim 24, wherein said proteasome inhibitor is selected from the group consisting of Velcade (bortezomib), and Kyprolis (carfilzomib).

30. The method of claim 24, wherein said CHK1 inhibitor is selected from the group consisting of TCS2312, PF-0047736, AZ07762, A-69002, and A-641397.

31. The method of claim 24, wherein the PARP inhibitor is selected from the group consisting of Olaparib, Talazoparib, ABT-888, (veliparib), KU-59436, AZD-2281, AG-014699, BSI-201, BGP-15, INO-1001, and ONO-2231.

32. The method of claim 24, wherein the tyrosine kinase inhibitor is selected from the group consisting of pexidartinib, entrectinib, matinib mesylate (ST1571; Gleevec), gefitinib (Iressa), erlotinib (OSI-1774; Tarceva), lapatinib (GW-572016), canertinib (CI-1033), semaxinib (SU5416), vatalanib (PTK787/ZK222584), sorafenib (BAY 43-9006), sutent (SU11248), and leflunomide (SU101).

33. The method of claim 24, wherein the CSF-1/CSF-1R inhibitor is selected from the group consisting of CSF-1R kinase inhibitor, an antibody that binds CSF-1R and is capable of blocking binding of CSF-1 to CSF-1R and IL-34 to CSF-1R.

34. The method of claim 24, wherein the CSF-1R kinase inhibitor is selected from the group consisting of imatinib, nilotinib and PLX3397.

35. The method of claim 14, wherein said radiation therapy is selected from the group consisting of X-rays, ion beams, electron beams, gamma-rays, UV-rays, decay of a radioactive isotope, and any combination thereof.

36. The method of claim 14, wherein said surgery is a tumor resection.

37. The method of claim 1, wherein said cancer is lung cancer selected from the group consisting of non-small cell lung cancer, pancreatic cancer, breast cancer, liver cancer, multiple myeloma, melanoma, leukemia, central nervous system cancer, stomach cancer, prostate, colon cancer, colorectal cancer, brain cancer, gastrointestinal cancer, gastric cancer, ovarian cancer, renal cancer, skin cancer, osteosarcoma, endometrial cancer, esophageal cancer, kidney cancer, prostate cancer, thyroid cancer, neuroblastoma, neurofibroma, glioma, glioblastoma, glioblastoma multiforme, stomach cancer, bladder cancer, head and neck cancer, cervical cancer, giant cell tumor of the tendon sheath, tenosynovial giant cell tumor, pigmented villonodular synovitis, cancers in which myeloid cells are involved, cancers in which myeloid cells are recruited and cancer cachexia.

38. The method of claim 1, wherein said at least one peptide inhibitor comprises a variant peptide sequence.

39. The method of claim 1, wherein said TREM-1/DAP-12 activity is selected group the group consisting of signaling and activation.

40. The method of claim 38, wherein said variant peptide sequence comprises at least one D-amino acid.

41. The method of claim 38, wherein said variant peptide sequence is a cyclic peptide.

42. The method of claim 38, wherein said variant peptide sequence is derived from transmembrane domain sequences of human or animal TREM-1 and/or its signaling subunit, DAP-12, and any combination thereof.

43. The method of claim 39, wherein said variant peptide sequence is selected group the group consisting of LR12, LP17 and a combination thereof.

44. The method of claim 1, wherein said method further comprises administering to said subject at least one antibody or fragment thereof, that specifically binds to TREM-1/DAP-12.

45. The method of claim 44, wherein said antibody or fragment thereof reduces TREM-1/DAP-12 activity.

46. The method of claim 2, wherein said subject is diagnosed prior to said administering said first dose.

47. The method of claim 46, wherein said subject is diagnosed after said administering said first dose.

48. The method of claim 47, wherein said diagnosis is selected from the group consisting of determining cancer progression, determining a result of cancer treatment. determining results of inhibiting TREM-1-mediated cell activation and reducing tumor growth.

49. The method of claim 46, wherein said diagnosis comprises isolating a biological sample from said subject.

50. The method of claim 49, wherein said diagnosis is based on expression levels of a marker selected from the group consisting of CSF-1, CSF-1R, IL-6, TREM-1, CD68 or any combination thereof.

51. The method of claim 50, wherein said diagnosis is based on the number of CD68 positive cells in said sample.

52. The method of claim 50, wherein said diagnosis is based on a response to said at least one peptide inhibitor selected from the group consisting of a higher expression level of a marker selected from the group consisting of CSF-1, CSF-1R, IL-6, TREM-1, CD68, a higher number of CD68-positive cells, and any combination thereof.

53. The method of claim 1, wherein said method further comprises: administering to said subject an amount of said at least one said peptide inhibitor that binds TREM-1 and is conjugated to at least one imaging probe; imaging at least a portion of said subject; detecting said imaging probe, wherein the location and amount of said imaging probe correlates with the TREM-1 expression levels in said cancer.

54. The method of claim 52, wherein higher TREM-1 expression levels predict a better response to said peptide inhibitor.

55. The method of claim 53, wherein said an imaging probe is selected from the group consisting of Gd(III), Mn(II), Mn(III), Cr(II), Cr(III), Cu(II), Fe (III), Pr(III), Nd(III) Sm(III), Tb(III), Yb(III) Dy(III), Ho(III), Eu(II), Eu(III), and Er(III), Tl²⁰¹, K⁴², In¹¹¹, Fe.⁵⁹, Tc^{99m}, Cr⁵¹, Ga⁶⁷, Ga⁶⁸, Cu⁶⁴, Rb⁸², Mo⁹⁹, Dy¹⁶⁵, Fluorescein, Carboxyfluorescein, Calcein, F¹⁸, Xe¹³³, I¹²⁵, I¹³¹, I¹²³, P³², C¹¹, N¹³, O¹⁵, Br⁷⁶, Kr⁸¹, Diatrizoate, Metrizoate, Isopaque, Ioxaglate, Iopamidol, Iohexol, Iodixanol, or a combination thereof.

56. A method for treating cancer in a subject, said method comprising administering to said subject a therapeutically effective amount of at least one isolated antibody or fragment thereof, that specifically binds TREM-1/DAP-12 for inhibiting the TREM-1/DAP-12 signaling pathway together with a therapeutically amount of a therapeutic selected from the group consisting of an anticancer vaccine, an anticancer immunotherapy agent, anti-cancer immunomodulatory agent, an additional anticancer therapeutic, radiation therapy, surgery or a combination thereof.
